(19) United States
(12) Patent Application Publication
(10) Pub. No.: US 2026/0242779 A1
(43) Pub. Date: Aug. 20, 2026

(54) CRISPR-RELATED METHODS AND COMPOSITIONS TARGETING LOW-DENSITY LIPOPROTEIN RECEPTOR (LDLR)

(71) Applicant: Editas Medicine, Inc., Cambridge, MA (US)

(72) Inventors: Paul Joseph Wrighton, Cambridge, MA (US); Benjamin Aryeh Diner, Cambridge, MA (US); Xin Jenny Xie, Cambridge, MA (US); Linda C. Burkly, Cambridge, MA (US); Tanya Marie Teslovich, Cambridge, MA (US); Eugenio Marco Rubio, Cambridge, MA (US); Meng Wu, Cambridge, MA (US); Heather Dawn MacLeod, Cambridge, MA (US); Georgia Giannoukos, Cambridge, MA (US)

(21) Appl. No.: 19/541,682

(22) Filed: Feb. 17, 2026

Related U.S. Application Data
(63) Continuation of application No. PCT/US2026/011325, filed on Jan. 15, 2026.
(60) Provisional application No. 63/745,724, filed on Jan. 15, 2025.
(60) Provisional application No. 63/805,160, filed on May 13, 2025.
(60) Provisional application No. 63/810,482, filed on May 22, 2025.
(60) Provisional application No. 63/872,843, filed on Aug. 29, 2025.
(60) Provisional application No. 63/872,782, filed on Aug. 29, 2025.
(60) Provisional application No. 63/894,039, filed on Oct. 6, 2025.
(60) Provisional application No. 63/894,034, filed on Oct. 6, 2025.
(60) Provisional application No. 63/911,882, filed on Nov. 5, 2025.
(60) Provisional application No. 63/911,907, filed on Nov. 5, 2025.
(60) Provisional application No. 63/920,708, filed on Nov. 19, 2025.
(60) Provisional application No. 63/928,124, filed on Dec. 1, 2025.
(60) Provisional application No. 63/928,177, filed on Dec. 1, 2025.

(51) Int. Cl. C12N 15/11; A61K 31/7105; A61K 38/46; A61P 3/06; C12N 9/22; C12N 15/88; C12N 15/90

(52) U.S. Cl. CPC C12N 15/11; A61K 31/7105; A61K 38/465; A61P 3/06; C12N 9/226; C12N 15/88; C12N 15/907; C12N 2310/20; C12N 2310/315; C12N 2310/321; C12N 2310/322; C12N 2310/3231; C12N 2310/335; C12N 2310/351; C12N 2310/531

(57) ABSTRACT

The present disclosure relates to genome editing systems and components for targeting, editing, and/or modulating the expression of a target nucleic acid sequence of interest, e.g., an LDLR target nucleic acid sequence in the 3′ untranslated region (UTR) of the gene encoding the LDLR protein. The present disclosure is also directed to methods and applications thereof in connection with the treatment and/or management of hypercholesterolemia.

### RELATED APPLICATIONS

The instant application claims priority to U.S. Provisional Application No. 63/745,724, filed Jan. 15, 2025; U.S. Provisional Application No. 63/805,160, filed May 13, 2025; U.S. Provisional Application No. 63/810,482, filed May 22, 2025; U.S. Provisional Application No. 63/872,782, filed Aug. 29, 2025; U.S. Provisional Application No. 63/872,843, filed Aug. 29, 2025; U.S. Provisional Application No. 63/894,034, filed Oct. 6, 2025; U.S. Provisional Application No. 63/894,039, filed Oct. 6, 2025; U.S. Provisional Application No. 63/911,882, filed Nov. 5, 2025; U.S. Provisional Application No. 63/911,907, filed Nov. 5, 2025; U.S. Provisional Application No. 63/920,708 filed Nov. 19, 2025; U.S. Provisional Application No. 63/928,124 filed Dec. 1, 2025; and U.S. Provisional Application No. 63/928,177, filed Dec. 1, 2025.

### SEQUENCE LISTING

The application contains a Sequence Listing which has been submitted electronically in. XML format and is hereby incorporated by reference in its entirety. Said. XML copy, created on Mar. 31, 2026, is named “126454-03013.xml” and is 2,465,126 bytes in size. The sequence listing contained in this. XML file is part of the specification and is hereby incorporated by reference herein in its entirety.

### FIELD

The present disclosure is directed to CRISPR-related genome editing systems and components for targeting, editing and/or modulating the expression of a low-density lipoprotein receptor (LDLR) target nucleic acid sequence of interest, e.g., an LDLR target nucleic acid sequence in the 3′ untranslated region (UTR) of the gene encoding the LDLR protein. The present disclosure is also directed to methods and applications thereof in connection with the treatment and/or management of hypercholesterolemia.

### BACKGROUND

Familial hypercholesterolemia (FH), which can be heterozygous (HeFH) or homozygous (HoFH), is characterized by high levels of low-density lipoprotein cholesterol (LDL-C) in the blood (≥160 mg/dL in people <20 years old, or ≥190 in people ≥20 years old) and an increased risk of atherosclerotic cardiovascular disease (ASCVD). FH has been associated with the formation of atherosclerotic plaques in the blood vessels that lead to coronary artery disease (CAD) and/or major adverse cardiovascular events (MACE) including, heart attack, stroke, aortic stenosis, peripheral vascular disease, and renal dysfunction. The current standard of care (SOC) for FH is lipoprotein apheresis, mitigation of other MACE risk factors, and drugs (e.g., statins, PCSK9 inhibitors), and fewer than 25% of patients achieve a target LDL of <70 mg/dL. Therapies employing antisense oligonucleotides (ASO) and small interfering RNA (siRNA) have met with many challenges, including tolerability, dosing inconsistencies, area under-the-curve (AUC) reductions and poor medication adherence by the patient. There is therefore an unmet need in the art for the treatment and/or management of diseases, e.g., alternate approaches that address hypercholesterolemia to reduce the risk of MACE in an individual or treat an individual who has suffered from a MACE.

In addition to elevated LDL-C, up to 50% of patients with HeFH also present with elevated lipoprotein(a) (Lp(a)), an independent ASCVD risk factor. High levels of Lp(a) (>150 mg/dL) have been associated with the formation of atherosclerotic plaques in the blood vessels that lead to MACE. The presence of elevated LDL-C and Lp(a) exert a compounding effect on ASCVD risk, highlighting the need for aggressive LDL-lowering therapeutic strategies aimed at lowering both LDL-C and Lp(a) for treating familial hypercholesterolemia to reduce the risk of MACE. See, for example, WO2025/166323, the entire contents of which are expressly incorporated by reference herein in their entirety.

The human LDLR gene is located on Chromosome 19. The LDLR gene (ENSG00000130164) comprises 18 exons that encode the LDLR protein. Expression of LDLR, which primarily occurs on liver cells (hepatocytes), facilitates absorption of LDL into cells, thereby decreasing blood levels of LDL to maintain normal levels of 75-150 mg/dL. Individuals with a naturally-occurring 2.5 kb deletion in the 3′ UTR of LDLR express high levels of LDLR and exhibit low serum LDL levels (22-44 mg/dL). In contrast, LDLR deficiencies are associated with increased serum LDL. Individuals with Familial Hypercholesterolemia (FH) carry at least one loss-of-function variant in at least one of several genes, including, e.g., LDLR, APOB, LDLRAP 1, and PCSK9. These individuals have increased plasma levels of total cholesterol (250-450 mg/dL) and are at higher risk of developing atherosclerotic cardiovascular disease, as compared to individuals with wild-type LDLR, APOB, LDLRAP1, and PCSK9. Currently there are no treatments available that genetically correct the decreased function of LDLR in these patients.

### SUMMARY

In one aspect, the present disclosure provides a composition comprising dual guide RNA (gRNA) molecules comprising targeting domains that flank the (AU-rich element 1) ARE1 element of a low-density lipoprotein receptor (LDLR) gene. In one embodiment, a composition comprises a first gRNA molecule comprising a first targeting domain that binds to a first target sequence of an LDLR gene, and a second gRNA molecule comprises a second targeting domain that binds to a second target sequence of an LDLR gene, wherein the first and second target sequences of the LDLR gene flank an ARE1 element of the LDLR gene. In one embodiment, the first gRNA molecule target sequence is located 5′ of the first AU-rich element (ARE1) of the Low Density Lipoprotein Receptor (LDLR) 3′ untranslated region (UTR); and the second gRNA molecule target sequence is located 3′ of the first AU-rich element (ARE1) of the LDLR 3′ UTR. In some embodiments, the first gRNA molecule target sequence is located within SEQ ID NO: 1482. In some embodiments, the first gRNA molecule target sequence comprises the ARE1 sequence (ATTTA). In some embodiments, the cleavage of the LDLR untranslated region (UTR) by a Cas protein complexed with such guide RNA molecules comprising targeting domains that bind to target sequences that flank the ARE1 element results in removal of the ARE1 element. Removal of the ARE1 element results in an increase in LDLR protein in the liver cell and/or liver, and a corresponding change of the levels of associated biomarkers, such as LDL-C, ApoB, and/or Lp(a). In other aspects, included herein are genome editing systems comprising the first and second gRNA molecules, RNP complexes comprising the first and second gRNA molecules, and delivery systems comprising the first and second gRNA molecules, each of which are described in more detail herein.

In some embodiments, administering the genome editing system, the RNP complex, or the delivery system disclosed herein induces an increase in an LDLR level in the subject, or in a cell, tissue, or fluid of the subject, e.g., in a liver cell or the liver of the subject, by at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, or at least 30-fold. In some embodiments, administering the genome editing system, the RNP complex, or the delivery system disclosed herein induces an increase in an LDLR level in the subject, or in a cell, tissue, or fluid of the subject, e.g., in a liver cell or the liver of the subject, by at least 1.5-fold to at least 31-fold. In some embodiments, administering the genome editing system, the RNP complex, or the delivery system disclosed herein induces an increase in an LDLR level in the subject, or in a cell, tissue, or fluid of the subject, e.g., in a liver cell or the liver of the subject, by about 1.5-fold to about 30-fold, about 5-fold to about 30-fold, about 10-fold to about 30-fold, about 20-fold to about 30-fold, about 1.5-fold to about 25-fold, about 1.5-fold to about 20-fold, about 1.5-fold to about 15-fold, about 1.5-fold to about 10-fold, about 2-fold to about 30-fold, about 5-fold to about 30-fold, about 10-fold to about 20-fold, about 10-fold to about 30-fold. In one embodiment, the increase in the LDLR level occurs after editing of the cell, as compared to a cell that has not been edited, or as compared to the liver cell or liver prior to editing in the cell or the subject.

In some embodiments, the compositions disclosed herein provide increased uptake of LDL-C by a liver cell or the liver by about 1.5-fold to about 5-fold, e.g., after editing. In some embodiments, the compositions disclosed herein provide increased uptake of LDL-C by a liver cell or the liver by at least 1.5-fold, at least 2-fold, at least 3-fold, at least 4-fold, at least 5-fold, e.g., after editing. In some embodiments, the compositions disclosed herein provide increased uptake of LDL-C by a liver cell or the liver by about 1.5-fold to about 5-fold, about 1.5-fold to about 3.5-fold, or about 3.2-fold, e.g., after editing.

In some embodiments, administering the genome editing system, the RNP complex, or the delivery system disclosed herein decreases an LDL-cholesterol (LDL-C) level in the subject, or in a cell, tissue, or fluid of the subject, e.g., in a liver cell or the liver of the subject, by at least about 50% to at least about 95%, e.g., relative to the LDL-C level in the cell, tissue, fluid, or subject prior to administering the genome editing system, the RNP complex, or the delivery system, or relative to a control cell, tissue, fluid, or subject. In some embodiments, the LDL-C level is decreased by about 50%, by about 60%, by about 65%, by about 70%, by about 75%, by about 80%, by about 85%, or by about 90%. In some embodiments, the LDL-C level is decreased by about 50% to about 85%, by about 50% to about 75%, by about 50% to about 70%, by about 55% to about 70%, or by about 55% to about 65%. In some embodiments, the LDL-C level is decreased by at least 50%, by at least 60%, by at least 65%, by at least 70%, by at least 75%, by at least 80%, by at least 85%, or by at least 90%.

In another embodiment, contacting the target cell with the genome editing system, the RNP complex, or the delivery system decreases an Apolipoprotein B (ApoB) level in the subject, or in a cell, tissue, or fluid of the subject, e.g., in a liver cell or the liver of the subject, by about at least about 30% to at least about 98%, e.g., relative to the ApoB level in the target cell prior to contacting the target cell with the genome editing system, the RNP complex, or the delivery system, or relative to a control cell. In some embodiments, a level of ApoB is decreased by about 50% to about 150%. In some embodiments, a level of ApoB is decreased by about 60% to about 140%, by about 70% to about 130%, by about 80% to about 120%, by about 90% to about 110%, or about 50%, about 60%, about 70%, about 80%, about 90%, about 100%, about 110%, or about 120%.

In another embodiment, administering the genome editing system, the RNP complex, or the delivery system to a subject decreases a Lipoprotein(a) (“Lp(a)”) level in the subject. In one embodiment, administering the genome editing system, the RNP complex, or the delivery system decreases a Lipoprotein(a) level in a cell, tissue, or fluid of the subject. In one embodiment, the Lp(a) level is decreased in the serum or plasma of the subject. In one embodiment, the cell is a liver cell. In one embodiment, the tissue is a liver tissue. In one embodiment, the Lp(a) level is decreased by about at least about 5% to at least about 98%. In one embodiment, the Lp(a) level is decreased by about 25% to about 98%. In one embodiment, the Lp(a) level is decreased by about 50% to about 98%. In one embodiment, the Lp(a) level is decreased by about 75% to about 98%. In one embodiment, the Lp(a) level is decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 95%, about 97%, or about 98%. In one embodiment, the Lp(a) level is decreased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 95%, at least 97%, or at least 98%. In any of the previous embodiments, the Lp(a) level is decreased relative to a Lp(a) level in the target cell, tissue, or fluid (e.g., serum or plasma of a subject) prior to administering the genome editing system, the RNP complex, or the delivery system to the subject. In any of the previous embodiments, the Lp(a) level is decreased relative to an Lp(a) control level, or relative to an Lp(a) level in a control cell. In some embodiments, a level of Lp(a) is decreased by about 50% to about 98%. In some embodiments, a level of Lp(a) is decreased by about 50% to about 95%. In some embodiments, a level of Lp(a) is decreased by about 50% to about 90%. In some embodiments, a level of Lp(a) is decreased by about 60% to about 98%, by about 70% to about 98%, by about 80% to about 98%, by about 90% to about 98%.

In certain embodiments, administering the genome editing system, the RNP complex, or the delivery system reduces a serum or plasma Lp(a) level in the subject to 100 mg/dL or lower.

In another aspect, the present disclosure provides a guide RNA (gRNA) molecule comprising a targeting domain that targets a target sequence of a low-density lipoprotein receptor (LDLR) gene, wherein the target sequence comprises any target sequence nucleotide sequence disclosed herein. In one aspect, the present disclosure provides a guide RNA (gRNA) molecule comprising a targeting domain that targets a target sequence of a low-density lipoprotein receptor (LDLR) gene, wherein the targeting domain comprises any targeting domain nucleotide sequence disclosed herein. In one embodiment, the targeting domain comprises a nucleotide sequence that is not SEQ ID NO: 323 or SEQ ID NO: 543. In another aspect, the present disclosure provides a guide RNA (gRNA) molecule comprising any gRNA sequence disclosed herein.

In another aspect, the present disclosure provides a guide RNA (gRNA) molecule comprising a targeting domain that targets a target sequence of a low-density lipoprotein receptor (LDLR) gene, wherein the target sequence comprises a nucleotide sequence selected from the group consisting of any one of SEQ ID NOs: 61-304, 549-606, 665-750, 837-865, 1317-1323, 1365-1392, and 1461. In one aspect, the present disclosure provides a guide RNA (gRNA) molecule comprising a targeting domain that targets a target sequence of a low-density lipoprotein receptor (LDLR) gene, wherein the targeting domain comprises a nucleotide sequence selected from the group consisting of any one of SEQ ID NOs: 305-548, 607-664, 751-836, 866-894, 1324-1330, 1393-1420, 1451-1456, 1462, 1464, 1466, 1468, 1470, 1474, and 1475. In one embodiment, the targeting domain comprises a nucleotide sequence that is not SEQ ID NO: 323 or SEQ ID NO: 543.

In one embodiment, the target sequence comprises a nucleotide sequence selected from the group consisting of any one of SEQ ID NOs: 61-74, 119, 123, 188, 275-302, 553, 589, 1317, 1318, and 1461; the targeting domain comprises a nucleotide sequence selected from the group consisting of any one of SEQ ID NOs: 305-318, 363, 367, 432, 519-542, 544-546, 611, 647, 1324, 1325, 1451-1454, 1456, 1462, 1464, 1466, 1470, 1474, and 1475; and/or the gRNA molecule comprises a nucleotide sequence selected from the group consisting of any one of SEQ ID NOs: 900-913, 958, 962, 1027, 1114-1138, 1139-1141, and 1148, 1184, 1331, 1332, 1449, 1450, 1457, 1458, 1460, 1463, 1465, 1467, 1471-1473, and 1476-1479.

In one aspect, disclosed herein is a gRNA molecule comprising a targeting domain that targets a target sequence of a low-density lipoprotein receptor (LDLR) gene, wherein the target sequence comprises a nucleotide sequence located within SEQ ID NO: 1482. In one embodiment, the target sequence comprises ATTTA.

In one embodiment, the gRNA molecule comprises one or more modifications. In one embodiment, the gRNA molecule comprises one or more modifications selected from the group consisting of a 5′ inverted thymidine (idT) modification, a 3′ idT modification, a 2′ fluoro modification, a 2′ O-methyl modification, a phosphorothioate linkage, a 3′ pseudoknot, a locked nucleic acid (LNA), and any combination thereof. In one embodiment, the gRNA molecule comprises one or more modifications selected from the group consisting of a 2′ O-methyl modification, a phosphorothioate linkage, and any combination thereof.

In one aspect, the present disclosure provides a composition comprising: a first gRNA molecule comprising a first targeting domain that binds to a first target sequence of an LDLR gene, and a second gRNA molecule comprising a second targeting domain that binds to a second target sequence of an LDLR gene, wherein the first and second target sequences of the LDLR gene flank the first AU-rich element (ARE1 element) of the 3′ untranslated region (UTR) of the LDLR gene. In one embodiment, the first targeting domain comprises a sequence selected from the group consisting of any one of SEQ ID NOs: 305-318, 1451, and 1454, and wherein the second targeting domain comprises a sequence selected from the group consisting of any one of SEQ ID NOs: 519-542, 544-546, 1456, 1470, 1474, and 1475. In one embodiment, the first gRNA molecule comprises a sequence selected from the group consisting of any one of SEQ ID NOs: 900-913 and 1476-1479 and wherein the second gRNA molecule comprises a sequence selected from the group consisting of any one of SEQ ID NOs: 1114-1137, 1139-1141, 1449, 1450, 1460, and 1471.

In one embodiment, the composition increases LDLR expression in a target cell by at least about 2-fold as compared to LDLR expression in a control cell or in the target cell prior to contacting with the composition. In one embodiment, the composition increases LDLR expression in a target cell by at least about 3-fold as compared to LDLR expression in a control cell or in the target cell prior to contacting with the composition. In one embodiment, the composition increases LDLR expression in a target cell by at least about 4-fold as compared to LDLR expression in a control cell or in the target cell prior to contacting with the composition. In one embodiment, the composition increases LDLR expression in a target cell by at least about 5-fold as compared to LDLR expression in a control cell or in the target cell prior to contacting with the composition. In one embodiment, the composition increases LDLR expression in a target cell by at least about 6-fold as compared to LDLR expression in a control cell or in the target cell prior to contacting with the composition. In one embodiment, the composition increases LDLR expression in a target cell by at least about 7-fold as compared to LDLR expression in a control cell or in the target cell prior to contacting with the composition. In one embodiment, the composition increases LDLR expression in a target cell by at least about 8-fold as compared to LDLR expression in a control cell or in the target cell prior to contacting with the composition. In one embodiment, the composition increases LDLR expression in a target cell by at least about 9-fold as compared to LDLR expression in a control cell or in the target cell prior to contacting with the composition. In one embodiment, the composition increases LDLR expression in a target cell by at least about 10-fold as compared to LDLR expression in a control cell or in the target cell prior to contacting with the composition. In one embodiment, the composition increases LDLR expression in a target cell by about 6-fold to about 11-fold as compared to LDLR expression in a control cell or in the target cell prior to contacting with the composition. In one embodiment, the composition increases LDLR expression in a target cell by about 7-fold to about 10-fold as compared to LDLR expression in a control cell or in the target cell prior to contacting with the composition. In one embodiment, the composition increases LDLR expression in a target cell by about 8-fold to about 10-fold as compared to LDLR expression in a control cell or in the target cell prior to contacting with the composition. In one embodiment, the composition increases LDLR expression in a target cell by about 9-fold to about 10-fold as compared to LDLR expression in a control cell or in the target cell prior to contacting with the composition. In one embodiment, the composition increases LDLR expression in a target cell by about 9-fold to about 11-fold as compared to LDLR expression in a control cell or in the target cell prior to contacting with the composition.

In one aspect, the present disclosure provides a genome editing system comprising at least one gRNA molecule as disclosed herein, or a nucleic acid encoding the gRNA molecule, and an RNA-guided nuclease, or a nucleic acid encoding the RNA-guided nuclease. In one embodiment, the genome editing system comprises a first gRNA molecule as herein disclosed, a second gRNA molecule as herein disclosed; and a RNA-guided nuclease or a nucleic acid encoding the RNA-guided nuclease, wherein the first gRNA molecule and the second gRNA molecule do not have the same nucleotide sequence.

In one aspect, the present disclosure provides a genome editing system comprising: a composition as herein disclosed, or a nucleic acid molecule encoding the first gRNA molecule and the second gRNA molecule of the composition as herein disclosed; and an RNA-guided nuclease, or a nucleic acid encoding the RNA-guided nuclease. In one embodiment, the nucleic acid encoding the RNA-guided nuclease is an RNA. In one embodiment, the RNA-guided nuclease is selected from the group consisting of Cas9, Cas12a (Cpf1), Cas12b, Cas12c, Cas12h, Cas12i, CasX, CasY, and CasΦ. In one embodiment, the RNA-guided nuclease is a Cas9 nuclease. In one embodiment, the Cas9 nuclease is a Streptococcus pyogenes Cas9 (SpCas9) nuclease. In one embodiment, the RNA-guided nuclease is a Cas12a nuclease. In one embodiment, the Cas12a nuclease is a modified Cas12a nuclease. In one embodiment, the modified Cas12a nuclease is a modified Acidaminococcus sp. Cas12a (AsCas12a) nuclease.

In one embodiment, the RNA-guided nuclease comprises an amino acid sequence selected from the group consisting of SEQ ID NOs: 1338-1347. In one embodiment, the nucleic acid encoding the RNA-guided nuclease comprises a nucleotide sequence selected from the group consisting of SEQ ID NOs: 1350-1359 and 1480.

In one embodiment, the genome editing system comprises a first gRNA molecule as disclosed herein, a second gRNA molecule as disclosed herein, and a RNA-guided nuclease or a nucleic acid encoding the RNA-guided nuclease, wherein the first gRNA molecule and the second gRNA molecule do not have the same nucleotide sequence.

In one aspect, the present disclosure provides a genome editing system comprising a first gRNA molecule comprising a targeting domain selected from the group consisting of SEQ ID NOs: 305-318 and 1451-1454, a second gRNA molecule comprising a targeting domain selected form the group consisting of SEQ ID NOs: 519-542, 544-546, 1456, 1470, 1474, and 1475, and an RNA encoding an RNA-guided nuclease. In one embodiment, the first gRNA molecule comprises a sequence selected from the group consisting of any one of SEQ ID NOs: 900-913 and 1476-1479 and wherein the second gRNA molecule comprises a sequence selected from the group consisting of any one of SEQ ID NOs: 1114-1137, 1139-1141, 1449, 1450, 1460, and 1471.

In one embodiment, the first gRNA molecule targeting domain and the second gRNA molecule targeting domain comprise, respectively, SEQ ID NO: 1452 and SEQ ID NO: 1475; SEQ ID NO: 305 and SEQ ID NO: 363, SEQ ID NO: 305 and SEQ ID NO: 367, SEQ ID NO: 305 and SEQ ID NO: 432, SEQ ID NO: 305 and SEQ ID NO: 519, SEQ ID NO: 305 and SEQ ID NO: 521, SEQ ID NO: 305 and SEQ ID NO: 524, SEQ ID NO: 305 and SEQ ID NO: 526, SEQ ID NO: 305 and SEQ ID NO: 527, SEQ ID NO: 305 and SEQ ID NO: 528, SEQ ID NO: 305 and SEQ ID NO: 529, SEQ ID NO: 305 and SEQ ID NO: 530, SEQ ID NO: 305 and SEQ ID NO: 531, SEQ ID NO: 305 and SEQ ID NO: 532, SEQ ID NO: 305 and SEQ ID NO: 535, SEQ ID NO: 305 and SEQ ID NO: 536, SEQ ID NO: 305 and SEQ ID NO: 537, SEQ ID NO: 305 and SEQ ID NO: 540, SEQ ID NO: 305 and SEQ ID NO: 543, SEQ ID NO: 305 and SEQ ID NO: 544, SEQ ID NO: 305 and SEQ ID NO: 545, SEQ ID NO: 306 and SEQ ID NO: 363, SEQ ID NO: 306 and SEQ ID NO: 367, SEQ ID NO: 306 and SEQ ID NO: 432, SEQ ID NO: 306 and SEQ ID NO: 519, SEQ ID NO: 306 and SEQ ID NO: 521, SEQ ID NO: 306 and SEQ ID NO: 524, SEQ ID NO: 306 and SEQ ID NO: 526, SEQ ID NO: 306 and SEQ ID NO: 527, SEQ ID NO: 306 and SEQ ID NO: 528, SEQ ID NO: 306 and SEQ ID NO: 529, SEQ ID NO: 306 and SEQ ID NO: 530, SEQ ID NO: 306 and SEQ ID NO: 531, SEQ ID NO: 306 and SEQ ID NO: 532, SEQ ID NO: 306 and SEQ ID NO: 535, SEQ ID NO: 306 and SEQ ID NO: 536, SEQ ID NO: 306 and SEQ ID NO: 537, SEQ ID NO: 306 and SEQ ID NO: 540, SEQ ID NO: 306 and SEQ ID NO: 543, SEQ ID NO: 306 and SEQ ID NO: 544, SEQ ID NO: 306 and SEQ ID NO: 545, SEQ ID NO: 307 and SEQ ID NO: 363, SEQ ID NO: 307 and SEQ ID NO: 367, SEQ ID NO: 307 and SEQ ID NO: 432, SEQ ID NO: 307 and SEQ ID NO: 519, SEQ ID NO: 307 and SEQ ID NO: 521, SEQ ID NO: 307 and SEQ ID NO: 524, SEQ ID NO: 307 and SEQ ID NO: 526, SEQ ID NO: 307 and SEQ ID NO: 527, SEQ ID NO: 307 and SEQ ID NO: 528, SEQ ID NO: 307 and SEQ ID NO: 529, SEQ ID NO: 307 and SEQ ID NO: 530, SEQ ID NO: 307 and SEQ ID NO: 531, SEQ ID NO: 307 and SEQ ID NO: 532, SEQ ID NO: 307 and SEQ ID NO: 535, SEQ ID NO: 307 and SEQ ID NO: 536, SEQ ID NO: 307 and SEQ ID NO: 537, SEQ ID NO: 307 and SEQ ID NO: 540, SEQ ID NO: 307 and SEQ ID NO: 543, SEQ ID NO: 307 and SEQ ID NO: 544, SEQ ID NO: 307 and SEQ ID NO: 545, SEQ ID NO: 308 and SEQ ID NO: 363, SEQ ID NO: 308 and SEQ ID NO: 367, SEQ ID NO: 308 and SEQ ID NO: 432, SEQ ID NO: 308 and SEQ ID NO: 519, SEQ ID NO: 308 and SEQ ID NO: 521, SEQ ID NO: 308 and SEQ ID NO: 524, SEQ ID NO: 308 and SEQ ID NO: 526, SEQ ID NO: 308 and SEQ ID NO: 527, SEQ ID NO: 308 and SEQ ID NO: 528, SEQ ID NO: 308 and SEQ ID NO: 529, SEQ ID NO: 308 and SEQ ID NO: 530, SEQ ID NO: 308 and SEQ ID NO: 531, SEQ ID NO: 308 and SEQ ID NO: 532, SEQ ID NO: 308 and SEQ ID NO: 535, SEQ ID NO: 308 and SEQ ID NO: 536, SEQ ID NO: 308 and SEQ ID NO: 537, SEQ ID NO: 308 and SEQ ID NO: 540, SEQ ID NO: 308 and SEQ ID NO: 543, SEQ ID NO: 308 and SEQ ID NO: 544, SEQ ID NO: 308 and SEQ ID NO: 545, SEQ ID NO: 314 and SEQ ID NO: 363, SEQ ID NO: 314 and SEQ ID NO: 367, SEQ ID NO: 314 and SEQ ID NO: 432, SEQ ID NO: 314 and SEQ ID NO: 519, SEQ ID NO: 314 and SEQ ID NO: 521, SEQ ID NO: 314 and SEQ ID NO: 524, SEQ ID NO: 314 and SEQ ID NO: 526, SEQ ID NO: 314 and SEQ ID NO: 527, SEQ ID NO: 314 and SEQ ID NO: 528, SEQ ID NO: 314 and SEQ ID NO: 529, SEQ ID NO: 314 and SEQ ID NO: 530, SEQ ID NO: 314 and SEQ ID NO: 531, SEQ ID NO: 314 and SEQ ID NO: 532, SEQ ID NO: 314 and SEQ ID NO: 535, SEQ ID NO: 314 and SEQ ID NO: 536, SEQ ID NO: 314 and SEQ ID NO: 537, SEQ ID NO: 314 and SEQ ID NO: 540, SEQ ID NO: 314 and SEQ ID NO: 543, SEQ ID NO: 314 and SEQ ID NO: 544, SEQ ID NO: 314 and SEQ ID NO: 545, SEQ ID NO: 315 and SEQ ID NO: 363, SEQ ID NO: 315 and SEQ ID NO: 367, SEQ ID NO: 315 and SEQ ID NO: 432, SEQ ID NO: 315 and SEQ ID NO: 519, SEQ ID NO: 315 and SEQ ID NO: 521, SEQ ID NO: 315 and SEQ ID NO: 524, SEQ ID NO: 315 and SEQ ID NO: 526, SEQ ID NO: 315 and SEQ ID NO: 527, SEQ ID NO: 315 and SEQ ID NO: 528, SEQ ID NO: 315 and SEQ ID NO: 529, SEQ ID NO: 315 and SEQ ID NO: 530, SEQ ID NO: 315 and SEQ ID NO: 531, SEQ ID NO: 315 and SEQ ID NO: 532, SEQ ID NO: 315 and SEQ ID NO: 535, SEQ ID NO: 315 and SEQ ID NO: 536, SEQ ID NO: 315 and SEQ ID NO: 537, SEQ ID NO: 315 and SEQ ID NO: 540, SEQ ID NO: 315 and SEQ ID NO: 543, SEQ ID NO: 315 and SEQ ID NO: 544, SEQ ID NO: 315 and SEQ ID NO: 545, SEQ ID NO: 316 and SEQ ID NO: 363, SEQ ID NO: 316 and SEQ ID NO: 367, SEQ ID NO: 316 and SEQ ID NO: 432, SEQ ID NO: 316 and SEQ ID NO: 519, SEQ ID NO: 316 and SEQ ID NO: 521, SEQ ID NO: 316 and SEQ ID NO: 524, SEQ ID NO: 316 and SEQ ID NO: 526, SEQ ID NO: 316 and SEQ ID NO: 527, SEQ ID NO: 316 and SEQ ID NO: 528, SEQ ID NO: 316 and SEQ ID NO: 529, SEQ ID NO: 316 and SEQ ID NO: 530, SEQ ID NO: 316 and SEQ ID NO: 531, SEQ ID NO: 316 and SEQ ID NO: 532, SEQ ID NO: 316 and SEQ ID NO: 535, SEQ ID NO: 316 and SEQ ID NO: 536, SEQ ID NO: 316 and SEQ ID NO: 537, SEQ ID NO: 316 and SEQ ID NO: 540, SEQ ID NO: 316 and SEQ ID NO: 543, SEQ ID NO: 316 and SEQ ID NO: 544, SEQ ID NO: 316 and SEQ ID NO: 545, SEQ ID NO: 317 and SEQ ID NO: 363, SEQ ID NO: 317 and SEQ ID NO: 367, SEQ ID NO: 317 and SEQ ID NO: 432, SEQ ID NO: 317 and SEQ ID NO: 519, SEQ ID NO: 317 and SEQ ID NO: 521, SEQ ID NO: 317 and SEQ ID NO: 524, SEQ ID NO: 317 and SEQ ID NO: 526, SEQ ID NO: 317 and SEQ ID NO: 527, SEQ ID NO: 317 and SEQ ID NO: 528, SEQ ID NO: 317 and SEQ ID NO: 529, SEQ ID NO: 317 and SEQ ID NO: 530, SEQ ID NO: 317 and SEQ ID NO: 531, SEQ ID NO: 317 and SEQ ID NO: 532, SEQ ID NO: 317 and SEQ ID NO: 535, SEQ ID NO: 317 and SEQ ID NO: 536, SEQ ID NO: 317 and SEQ ID NO: 537, SEQ ID NO: 317 and SEQ ID NO: 540, SEQ ID NO: 317 and SEQ ID NO: 543, SEQ ID NO: 317 and SEQ ID NO: 544, or SEQ ID NO: 317 and SEQ ID NO: 545.

In one embodiment, the first gRNA molecule targeting domain and the second gRNA molecule targeting domain comprise, respectively, SEQ ID NO: 1452 and SEQ ID NO: 1475; SEQ ID NO: 305 and SEQ ID NO: 532; SEQ ID NO: 316 and SEQ ID NO: 531; SEQ ID NO: 317 and SEQ ID NO: 532; SEQ ID NO: 318 and SEQ ID NO: 532; SEQ ID NO 305 and SEQ ID NO: 544; SEQ ID NO: 316 and SEQ ID NO: 544; SEQ ID NO: 317 and SEQ ID NO: 544; or SEQ ID NO: 318 and SEQ ID NO: 544. In one embodiment, the first gRNA molecule and the second gRNA molecule comprise, respectively, SEQ ID NO: 1477 and SEQ ID NO: 1450; SEQ ID NO: 900 and SEQ ID NO: 1127; SEQ ID NO: 911 and SEQ ID NO: 1126; SEQ ID NO: 912 and SEQ ID NO: 1127; SEQ ID NO: 913 and SEQ ID NO: 1127; SEQ ID NO: 900 and SEQ ID NO: 1139; SEQ ID NO: 911 and SEQ ID NO: 1139; SEQ ID NO: 912 and SEQ ID NO: 1139; or SEQ ID NO: 913 and SEQ ID NO: 1139. In one embodiment, the first gRNA molecule and the second gRNA molecule comprise, respectively, targeting domain comprise, respectively, SEQ ID NO: 1452 and SEQ ID NO: 1475. In one embodiment, the first gRNA molecule and the second gRNA molecule comprise, respectively, SEQ ID NO: 1477 and SEQ ID NO: 1450.

In one embodiment, the first gRNA molecule comprises a targeting domain that targets a first target sequence, and wherein the second gRNA molecule comprises a targeting domain that targets a second target sequence, wherein the first target sequence and the second target sequence comprise a first and second nucleotide sequence, respectively, selected from the group consisting of SEQ ID NO: 73 and SEQ ID NO: 300; SEQ ID NO: 79 and SEQ ID NO: 299; SEQ ID NO: 553 and SEQ ID NO: 589; SEQ ID NO: 64 and SEQ ID NO: 277; SEQ ID NO: 64 and SEQ ID NO: 280; SEQ ID NO: 64 and SEQ ID NO: 284; SEQ ID NO: 64 and SEQ ID NO: 285; SEQ ID NO: 64 and SEQ ID NO: 300; SEQ ID NO: 70 and SEQ ID NO: 277; SEQ ID NO: 70 and SEQ ID NO: 280; SEQ ID NO: 70 and SEQ ID NO: 284; SEQ ID NO: 70 and SEQ ID NO: 285; SEQ ID NO: 70 and SEQ ID NO: 300; SEQ ID NO: 72 and SEQ ID NO: 277; SEQ ID NO: 72 and SEQ ID NO: 280; SEQ ID NO: 72 and SEQ ID NO: 284; SEQ ID NO: 72 and SEQ ID NO: 285; SEQ ID NO: 72 and SEQ ID NO: 300; SEQ ID NO: 73 and SEQ ID NO: 284; SEQ ID NO: 73 and SEQ ID NO: 285; and SEQ ID NO: 74 and SEQ ID NO: 300.

In one embodiment, the targeting domain comprises a first targeting domain and a second targeting domain, comprising a first and second nucleotide sequence, respectively, selected from the group consisting of SEQ ID NO: 1452 and SEQ ID NO: 1475; SEQ ID NO: 317 and SEQ ID NO: 544; SEQ ID NO: 1455 and SEQ ID NO: 1456; SEQ ID NO 1468 and SEQ ID NO: 1470; SEQ ID NO: 611 and SEQ ID NO: 647; SEQ ID NO: 308 and SEQ ID NO: 521; SEQ ID NO: 308 and SEQ ID NO: 524; SEQ ID NO: 308 and SEQ ID NO: 528; SEQ ID NO: 308 and SEQ ID NO: 529; SEQ ID NO: 308 and SEQ ID NO: 544; SEQ ID NO: 314 and SEQ ID NO: 521; SEQ ID NO: 314 and SEQ ID NO: 524; SEQ ID NO: 314 and SEQ ID NO: 28; SEQ ID NO: 314 and SEQ ID NO: 529; SEQ ID NO: 314 and SEQ ID NO: 544; SEQ ID NO: 316 and SEQ ID NO: 521; SEQ ID NO: 316 and SEQ ID NO: 521; SEQ ID NO: 316 and SEQ ID NO: 528; SEQ ID NO: 316 and SEQ ID NO: 529; SEQ ID NO: 316 and SEQ ID NO: 544; SEQ ID NO: 317 and SEQ ID NO: 528; SEQ ID NO: 317 and SEQ ID NO: 529; and SEQ ID NO: 318 and SEQ ID NO: 544.

In one embodiment, the first and second gRNA molecules comprise a first and second nucleotide sequence, respectively, selected from the group consisting of SEQ ID NO: 1477 and SEQ ID NO: 1450; SEQ ID NO: 900 and SEQ ID NO: 1139; SEQ ID NO: 1459 and SEQ ID NO: 1460; SEQ ID NO: 1469 and SEQ ID NO: 1471; SEQ ID NO: 1148 and SEQ ID NO: 1184; SEQ ID NO: 903 and SEQ ID NO: 1116; SEQ ID NO: 903 and SEQ ID NO: 1119; SEQ ID NO: 903 and SEQ ID NO: 1123; SEQ ID NO: 903 and SEQ ID NO: 1124; SEQ ID NO: 903 and SEQ ID NO: 1139; SEQ ID NO: 909 and SEQ ID NO: 1116; SEQ ID NO: 909 and SEQ ID NO: 1119; SEQ ID NO: 909 and SEQ ID NO: 1123; SEQ ID NO: 909 and SEQ ID NO: 1124; SEQ ID NO: 909 and SEQ ID NO: 1139; SEQ ID NO: 911 and SEQ ID NO: 1116; SEQ ID NO: 911 and SEQ ID NO: 1123; SEQ ID NO: 911 and SEQ ID NO: 1124; SEQ ID NO: 911 and SEQ ID NO: 1139; SEQ ID NO: 912 and SEQ ID NO: 1123; SEQ ID NO: 912 and SEQ ID NO: 1124; and SEQ ID NO: 913 and SEQ ID NO: 1139.

In one embodiment, the targeting domain comprises a first targeting domain and a second targeting domain, comprising a first and second nucleotide sequence, respectively, selected from the group consisting of SEQ ID NO: 1452 and SEQ ID NO: 1475. In one embodiment, the first and second gRNA molecules comprise a first and second nucleotide sequence, respectively, selected from the group consisting of SEQ ID NO: 1477 and SEQ ID NO: 1450. In one embodiment, the RNA guided nuclease is a Streptococcus pyogenes Cas9 (SpCas9) nuclease. In one aspect, the present disclosure provides a ribonucleoprotein (RNP) complex comprising the genome editing system as disclosed herein.

In one aspect, the present disclosure provides a delivery system for delivering the genome editing system as disclosed herein, wherein the delivery system comprises the at least one gRNA molecule and an RNA molecule encoding the RNA-guided nuclease. In one embodiment, the delivery system comprises a lipid nanoparticle (LNP) encapsulating the at least one gRNA molecule and the RNA molecule encoding the RNA-guided nuclease. In one embodiment, the LNP comprises an ionizable lipid, a polyethylene glycol (PEG) lipid, a helper lipid, a sterol, or any combination thereof.

In one embodiment, the genome editing system increases LDLR expression in a target cell by at least about 2-fold as compared to LDLR expression in a control cell or in the target cell prior to contacting with the genome editing system. In one embodiment, the genome editing system increases LDLR expression in a target cell by at least about 3-fold as compared to LDLR expression in a control cell or in the target cell prior to contacting with the genome editing system. In one embodiment, the genome editing system increases LDLR expression in a target cell by at least about 4-fold as compared to LDLR expression in a control cell or in the target cell prior to contacting with the genome editing system. In one embodiment, the genome editing system increases LDLR expression in a target cell by at least about 5-fold as compared to LDLR expression in a control cell or in the target cell prior to contacting with the genome editing system. In one embodiment, the genome editing system increases LDLR expression in a target cell by at least about 6-fold as compared to LDLR expression in a control cell or in the target cell prior to contacting with the genome editing system. In one embodiment, the genome editing system increases LDLR expression in a target cell by at least about 7-fold as compared to LDLR expression in a control cell or in the target cell prior to contacting with the genome editing system. In one embodiment, the genome editing system increases LDLR expression in a target cell by at least about 8-fold as compared to LDLR expression in a control cell or in the target cell prior to contacting with the genome editing system. In one embodiment, the genome editing system increases LDLR expression in a target cell by at least about 9-fold as compared to LDLR expression in a control cell or in the target cell prior to contacting with the genome editing system. In one embodiment, the genome editing system increases LDLR expression in a target cell by at least about 10-fold as compared to LDLR expression in a control cell or in the target cell prior to contacting with the genome editing system. In one embodiment, the genome editing system increases LDLR expression in a target cell by about 6-fold to about 11-fold as compared to LDLR expression in a control cell or in the target cell prior to contacting with the genome editing system. In one embodiment, the genome editing system increases LDLR expression in a target cell by about 7-fold to about 10-fold as compared to LDLR expression in a control cell or in the target cell prior to contacting with the genome editing system. In one embodiment, the genome editing system increases LDLR expression in a target cell by about 8-fold to about 10-fold as compared to LDLR expression in a control cell or in the target cell prior to contacting with the genome editing system. In one embodiment, the genome editing system increases LDLR expression in a target cell by about 9-fold to about 10-fold as compared to LDLR expression in a control cell or in the target cell prior to contacting with the genome editing system. In one embodiment, the genome editing system increases LDLR expression in a target cell by about 9-fold to about 11-fold as compared to LDLR expression in a control cell or in the target cell prior to contacting with the genome editing system.

In one aspect, the present disclosure provides a method of editing an LDLR gene in a target cell, the method comprising contacting the target cell with the genome editing system as disclosed herein, the RNP complex as disclosed herein, or the delivery system as disclosed herein. In one embodiment, the LNP comprises a targeting moiety. In one embodiment, the targeting moiety is one or more N-acetylgalactosamine (GalNAc) or GalNAc derivatives.

In one embodiment, the target cell is in vivo, ex vivo, or in vitro. In one embodiment, the target cell is in vivo. In one embodiment, the target cell is from a subject with heterozygous familial hypercholesterolemia (HeFH) or homozygous familial hypercholesterolemia (HoFH). In one embodiment, the target cell is a hepatocyte.

In one embodiment, contacting the target cell with the genome editing system, the RNP complex, or the delivery system increases an LDLR protein level in the target cell by at least about 5-fold to at least about 15-fold, relative to the LDLR protein level in the target cell prior to contacting the target cell with the genome editing system, the RNP complex, or the delivery system, or relative to a control cell. In one embodiment, the increase in LDLR protein level is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 18.5-fold, at least about 20-fold, at least about 25-fold, at least about 30 fold. In one embodiment, the increase in LDLR protein level is increased by at least about 9-fold. In one embodiment, the increase in LDLR protein level is increased by at least about 10-fold. In one embodiment, the increase in LDLR protein level is increased by at least about 6-fold to about 11-fold. In one embodiment, the increase in LDLR protein level is increased by at least about 9-fold to about 10-fold. In one embodiment, the increase in LDLR protein level is increased by at least about 6-fold to about 10-fold. In one embodiment, the increase in LDLR protein level is increased by at least about 7-fold to about 10-fold. In one embodiment, the increase in LDLR protein level is increased by at least about 8-fold to about 10-fold. In one embodiment, the increase in LDLR protein level is increased by at least about 9-fold to about 11-fold.

In one aspect, the present disclosure provides a method of treating a disease or a disorder in a subject, the method comprising administering to the subject the genome editing system as disclosed herein, the RNP complex as disclosed herein, or the delivery system as disclosed herein, thereby treating the disease or disorder. In one embodiment, the subject has heterozygous familial hypercholesterolemia (HeFH) or homozygous familial hypercholesterolemia (HoFH). In one embodiment, the disease or disorder is heterozygous familial hypercholesterolemia (HeFH) or homozygous familial hypercholesterolemia (HoFH). In one embodiment, administering the genome editing system, the RNP complex, or the delivery system increases an LDLR protein level in the subject, or in a cell, tissue, or fluid of the subject, by at least about 5-fold to at least about 15-fold, relative to the LDLR protein level in the cell, tissue, fluid, or subject prior to administering the genome editing system, the RNP complex, or the delivery system, or relative to a control cell, tissue, fluid, or subject. In one embodiment, the increase in LDLR protein level is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 18.5-fold, at least about 20-fold, at least about 25-fold, at least about 30 fold. In one embodiment, the increase in LDLR protein level is increased by at least about 9-fold. In one embodiment, the increase in LDLR protein level is increased by at least about 10-fold. In one embodiment, the increase in LDLR protein level is increased by at least about 6-fold to about 11-fold. In one embodiment, the increase in LDLR protein level is increased by at least about 9-fold to about 10-fold. In one embodiment, the increase in LDLR protein level is increased by at least about 6-fold to about 10-fold. In one embodiment, the increase in LDLR protein level is increased by at least about 7-fold to about 10-fold. In one embodiment, the increase in LDLR protein level is increased by at least about 8-fold to about 10-fold. In one embodiment, the increase in LDLR protein level is increased by at least about 9-fold to about 11-fold.

In one embodiment, administering the genome editing system, the RNP complex, or the delivery system decreases an LDL-cholesterol (LDL-C) level in the subject, or in a cell, tissue, or fluid of the subject, by at least about 50% to at least about 95%, relative to the LDL-C level in the cell, tissue, fluid, or subject prior to administering the genome editing system, the RNP complex, or the delivery system, or relative to a control cell, tissue, fluid, or subject. In one embodiment, contacting the target cell with the genome editing system, the RNP complex, or the delivery system decreases an Apolipoprotein B (ApoB) level in the subject, or in a cell, tissue, or fluid of the subject, e.g., in a liver cell or the liver of the subject, by about at least about 30% to at least about 98% relative to the ApoB level in the target cell prior to contacting the target cell with the genome editing system, the RNP complex, or the delivery system, or relative to a control cell.

In one embodiment, administering the genome editing system, the RNP complex, or the delivery system decreases an Lipoprotein(a) (Lp(a)) level in the subject, or in a cell, tissue, or fluid of the subject, e.g., in serum or plasma of a subject, or a liver cell or the liver of the subject, by about at least about 50% to at least about 95% relative to the Lp(a) level in the cell, tissue, or fluid of the subject prior to administering the genome editing system, the RNP complex, or the delivery system, or relative to a control. In one embodiment, the control is an Lp(a) serum or plasma level from a control subject or population of control subjects.

In one embodiment, the first gRNA molecule comprises a first targeting domain that targets a first target sequence, wherein the first target sequence is a first nucleotide sequence that is located 5′ to SEQ ID NO: 74 in the LDLR gene, and does not comprise SEQ ID NO: 74. In one embodiment, the first nucleotide sequence is SEQ ID NO: 73. In one embodiment, the second gRNA molecule comprises a second targeting domain that targets a second target sequence, wherein the second target sequence comprises a second nucleotide sequence selected from the group consisting of any one of SEQ ID NOs: 284, 285, 288, 289, or 300. In one embodiment, the second nucleotide sequence is SEQ ID NO: 300. In one embodiment, the first gRNA molecule comprises SEQ ID NO: 1477, and the second gRNA molecule comprises SEQ ID NO: 1450. In one embodiment, the first targeting domain comprises SEQ ID NO: 1452, and the second targeting domain comprises SEQ ID NO: 1475.

In one embodiment, contacting the target cell with, or administering to the subject, the genome editing system, the RNP complex, or the delivery system increases an LDLR protein level in the target cell, or in a cell, tissue, or fluid of the subject, by at least about 2-fold, 3-fold, 4-fold, 5-fold, or 6-fold relative to a LDLR protein level in a target cell, or in a cell, tissue, or fluid of the subject, contacted with, or administered, a second genome editing system, RNP complex or delivery system which comprises a third gRNA molecule that comprises a third targeting domain that targets a third sequence, wherein the third target sequence is a third nucleotide sequence that comprises SEQ ID NO: 74, or that is located 3′ to SEQ ID NO: 74 in the LDLR gene. In one embodiment, the second genome editing system, RNP complex, or delivery system further comprises a fourth gRNA molecule that is the same as the second gRNA molecule. In one embodiment, the LDLR protein level is increased by at least about 2-fold. In one embodiment, the LDLR protein level is increased by at least about 3-fold. In one embodiment, the LDLR protein level is increased by at least about 4-fold. In one embodiment, the LDLR protein level is increased by at least about 5-fold. In one embodiment, the LDLR protein level is increased by at least about 6-fold. In one embodiment, the LDLR protein level is increased by about 2-fold to about 6-fold. In one embodiment, the LDLR protein level is increased by about 6-fold.

### BRIEF DESCRIPTION OF THE DRAWINGS

The accompanying drawings are intended to provide illustrative and schematic rather than comprehensive examples of certain aspects and embodiments of the present disclosure. The drawings are not intended to be limiting or binding to any particular theory or model. Without limiting the foregoing, nucleic acids and polypeptides can be depicted as linear sequences, or as schematic two- or three-dimensional structures; these depictions are intended to be illustrative rather than limiting or binding to any particular model or theory regarding their structure.

FIG. 1 depicts the architecture of the 3′ UTR of the human LDLR gene, including select regulatory sites and a naturally-occurring 2.5 kb deletion described in Example 1.

FIGS. 2A-2B depict the coverage of the human LDLR 3′ UTR by the gRNAs provided by the present disclosure for SpCas9 and AsCas12a (WT, RR, and RVR) described in Example 1. The numbers on the graphic indicate DNA target sequence SEQ ID NOs.

FIG. 3 is a graph demonstrating ILL-seq data from duplicate experiments (dashed and open circles represent biological replicates) editing HepG2 cells with single disclosed AsCas12a (WT) RNPs, as described in Example 2. The x-axis indicates DNA target sequence SEQ ID NO.

FIG. 4 is a graph demonstrating UDiTaS data from duplicate experiments editing HepG2 cells with single disclosed AsCas12a (WT) RNPs including a gRNA targeting the indicated DNA target sequence, as described in Example 2.

FIG. 5 is a graph demonstrating UDiTaS data from duplicate experiments editing HepG2 cells with dual disclosed AsCas12a (WT) RNPs including gRNAs targeting the indicated DNA target sequences, as described in Example 2.

FIG. 6 is a graph demonstrating the surface expression level of LDLR on HepG2 cells edited with dual AsCas12a (WT) RNPs including gRNAs targeting the indicated combinations of DNA target sequences, assessed by flow cytometry as described in Example 2.

FIG. 7A is a graph demonstrating dose-dependent effects of total RNA (i.e., the sum of mRNA and gRNA) on frequency of edits to the LDLR 3′ UTR in HepG2 cells edited with dual SpCas9 gRNAs, delivered by LNP, and assessed by UDiTaS as described in Example 3.

FIG. 7B is a graph demonstrating dose-dependent effects of total RNA (i.e., the sum of mRNA and gRNA) on LDLR mRNA levels in HepG2 cells edited with dual SpCas9 gRNAs, delivered by LNP, and assessed by RTddPCR as described in Example 3.

FIG. 8A is a graph demonstrating dose-dependent effects of total RNA (i.e., the sum of mRNA and gRNA) on total LDLR protein levels in HepG2 cells edited with dual SpCas9 gRNAs, delivered by LNP, and assessed by ELISA as described in Example 3.

FIG. 8B is a graph demonstrating dose-dependent effects of total RNA (i.e., the sum of mRNA and gRNA) on LDL-C uptake by HepG2 cells edited with dual SpCas9 gRNAs, delivered by LNP, and assessed by flow cytometry as described in Example 3.

FIG. 8C is a histogram demonstrating the effect of 4E-4 mg/mL total RNA (i.e., the sum of mRNA and gRNA) on LDL-C uptake by HepG2 cells edited with dual SpCas9 gRNAs, delivered by LNP, and assessed by flow cytometry as described in Example 3.

FIG. 9A is a graph demonstrating dose-dependent effects of total RNA (i.e., the sum of mRNA and gRNA) on frequency of edits to LDLR 3′ UTR in primary human hepatocytes (PHHs) edited with dual SpCas9 gRNAs, delivered by LNP, and assessed by UDiTaS as described in Example 4.

FIG. 9B is a graph demonstrating dose-dependent effects of total RNA (i.e., the sum of mRNA and gRNA) on LDLR mRNA levels in primary human hepatocytes (PHHs) edited with dual SpCas9 gRNAs, delivered by LNP, and assessed by RTddPCR as described in Example 4.

FIG. 9C is a graph demonstrating dose-dependent effects of total RNA (i.e., the sum of mRNA and gRNA) on total LDLR protein levels in primary human hepatocytes (PHHs) edited with dual SpCas9 gRNAs, delivered by LNP, and assessed by ELISA as described in Example 4.

FIG. 10A is a graph demonstrating dose-dependent effects of total RNA (i.e., the sum of mRNA and gRNA) on total LDLR protein levels in HepG2 cells edited with dual-guide AsCas12a (WT) systems including the indicated combinations of DNA target sequences. AsCas12a mRNA and gRNA pairs were delivered by LNP and LDLR protein levels were assessed by ELISA as described in Example 5.

FIG. 10B is a graph demonstrating dose-dependent effects of total RNA (i.e., the sum of mRNA and gRNA) on total LDLR protein levels in primary human hepatocytes (PHHs) edited with dual-guide AsCas12a (WT) systems including the indicated combinations of DNA target sequences. AsCas12a mRNA and gRNA pairs were delivered by LNP and LDLR protein levels were assessed by ELISA described in Example 5.

FIG. 11A is a graph demonstrating the frequency of indel generation in primary human hepatocytes (PHHs) cotransfected with mRNA encoding SpCas9 and single gRNAs proximal to the stop codon assessed by UDiTaS as described in Example 6. The x-axis depicts DNA target sequence and cut position within the LDLR 3′ UTR (in parentheses).

FIG. 11B is a graph demonstrating the frequency of indel generation in primary human hepatocytes (PHHs) cotransfected with mRNA encoding SpCas9 and single gRNAs distal to the stop codon assessed by UDiTaS as described in Example 6. The x-axis depicts DNA target sequence and cut position within the LDLR 3′ UTR (in parentheses).

FIGS. 12A-12D are graphs demonstrating the fold change in LDLR protein expression in primary human hepatocytes (PHHs) cotransfected with mRNA encoding SpCas9 and gRNA pairs, assessed by ELISA, in which one gRNA is always the specified “anchor gRNA,” which is proximal to the stop codon, as described in Example 6. The x-axis depicts DNA target sequence and cut position within the LDLR 3′ UTR (in parentheses).

FIGS. 13A-13D are graphs demonstrating the fold change in LDLR protein expression in primary human hepatocytes (PHHs) cotransfected with mRNA encoding SpCas9 and gRNA pairs, assessed by ELISA, in which one gRNA is always the specified “anchor gRNA,” which is distal to the stop codon and precedes the polyA sites, as described in Example 6. The x-axis depicts DNA target sequence and cut position within the LDLR 3′ UTR (in parentheses).

FIGS. 14A-14D are graphs demonstrating the fold change in LDLR protein expression in primary human hepatocytes (PHHs) cotransfected with mRNA encoding SpCas9 and gRNA pairs, assessed by ELISA, in which one gRNA is always the specified “anchor gRNA,” which is distal to the stop codon and follows the polyA sites, as described in Example 6. The x-axis depicts DNA target sequence and cut position within the LDLR 3′ UTR (in parentheses).

FIG. 15 is a chart demonstrating the fold upregulation of total LDLR protein levels in primary human hepatocytes (PHHs) cotransfected with mRNA encoding SpCas9 and combinations of gRNAs targeting the indicated DNA target sequences assessed by ELISA as described in Example 6.

FIG. 16A is a graph demonstrating the frequency and type of edits produced by AsCas12a (WT) mRNA and gRNA pairs delivered by LNP in a population of primary human hepatocytes (PHHs) assessed by UDiTaS as described in Example 7. The indicated gRNA pair DNA target sequences are described in Table 24.

FIG. 16B is a graph demonstrating the fold change in total LDLR protein levels in primary human hepatocytes (PHHs) with AsCas12a (WT) mRNA and gRNA pairs delivered by LNP assessed by ELISA as described in Example 7. The indicated gRNA pair DNA target sequences are described in Table 24.

FIG. 17A is a graph demonstrating dose-dependent effects of total RNA (i.e., the sum of mRNA and gRNA) on frequency and type of editing in a population of primary human hepatocytes (PHHs) edited with dual AsCas12a (WT) gRNAs delivered by LNP as assessed by UDiTaS, as described in Example 7. The indicated gRNA pair DNA target sequences are described in Table 24.

FIG. 17B is a graph demonstrating dose-dependent effects of total RNA (i.e., the sum of mRNA and gRNA) on total LDLR protein levels in primary human hepatocytes (PHHs) edited with dual AsCas12a (WT) gRNAs delivered by LNP as assessed by ELISA, as described in Example 7. The indicated gRNA pair DNA target sequences are described in Table 24.

FIG. 17C is a graph demonstrating the correlation of frequency of edits (assessed by UDiTaS) and LDLR fold up-regulation (assessed by ELISA) in primary human hepatocytes (PHHs) edited with dual AsCas12a (WT) gRNAs delivered by LNP as described in Example 7. The indicated gRNA pair DNA target sequences are described in Table 24.

FIG. 18A is a graph demonstrating the frequency and type of editing in a population of primary human hepatocytes (PHHs) edited with dual SpCas9 or AsCas12a (WT) gRNAs delivered by LNP as assessed by UDiTaS, as described in Example 8. The indicated gRNA pair DNA target sequences are described in Table 25A.

FIG. 18B is a graph demonstrating fold change in total LDLR protein levels in primary human hepatocytes (PHHs) edited with dual SpCas9 or AsCas12a (WT) gRNAs delivered by LNP as assessed by ELISA, as described in Example 8. The indicated gRNA pair DNA target sequences are described in Table 25A.

FIG. 18C is a graph demonstrating the correlation of frequency of edits (assessed by UDiTaS) and LDLR fold up-regulation (assessed by ELISA) in primary human hepatocytes (PHHs) edited with dual SpCas9 or AsCas12a (WT) gRNAs delivered by LNP as described in Example 8. The indicated gRNA pair DNA target sequences are described in Table 25A.

FIG. 19 depicts the architecture of the 3′ UTR of the LDLR gene, including select regulatory sites and the Proximal and Proximal-ARE deletions induced by gRNA pairs disclosed herein.

FIG. 20A is a chart demonstrating the fold change in total LDLR protein levels in primary human hepatocytes (PHHs) edited with SpCas9 gRNA pairs delivered by LNP and targeting the indicated combinations of DNA target sequences, assessed by ELISA as described in Example 9.

FIG. 20B is a graph demonstrating the fold-change in total LDLR protein levels in primary human hepatocytes (PHHs) after editing with SpCas9 gRNA pairs delivered by LNP and targeting the indicated combinations of DNA target sequences, assessed by ELISA as described in Example 9.

FIG. 20C is a graph demonstrating total LDLR protein levels in primary human hepatocytes (PHHs) edited with SpCas9 gRNA pairs delivered by LNP and targeting the indicated combinations of DNA target sequences, assessed by ELISA as described in Example 9.

FIG. 20D is a graph demonstrating total LDLR protein levels in primary human hepatocytes (PHHs) edited with the Proximal-ARE SpCas9 gRNA pair delivered by LNP and assessed by ELISA, as described in Example 9.

FIG. 20E is a graph demonstrating the change in LDLR mRNA levels in HepG2 cells after editing with the indicated combination of SpCas9 gRNA pairs delivered by LNP and targeting the gRNA target sequences or a non-targeting control, and subsequent treatment with Actinomycin D (ActD), as described in Example 9.

FIG. 21 is a graph demonstrating frequency and type of editing in a population of primary human hepatocytes (PHHs) edited with SpCas9 gRNA pairs delivered by LNP and targeting the indicated combinations of DNA target sequences, as assessed by UDiTaS as described in Example 9.

FIG. 22A is a chart demonstrating the percent of mRNAs from primary human hepatocytes (PHHs) exhibiting large deletions after editing with SpCas9 gRNA pairs delivered by LNP and targeting the indicated combinations of DNA target sequences, as assessed by RT-ddPCR as described in Example 9.

FIG. 22B is a chart demonstrating the sum percent of mRNAs from primary human hepatocytes (PHHs) exhibiting large deletions and inversions after editing with SpCas9 gRNA pairs delivered by LNP and targeting the indicated combinations of DNA target sequences, as assessed by RT-ddPCR as described in Example 9.

FIG. 22C is a graph demonstrating the frequency of functional edits in in primary human hepatocytes (PHHs) after editing with SpCas9 gRNA pairs delivered by LNP and targeting the indicated combinations of DNA target sequences, assessed by ELISA as described in Example 9.

FIG. 22D is a graph demonstrating the fold change in LDLR mRNA levels in primary human hepatocytes (PHHs) treated with varying doses of gRNA pairs delivered by LNP and targeting the indicated combinations of SpCas9 gRNA target sequences, as assessed by RT-ddPCR as described in Example 9.

FIG. 23A is a graph demonstrating the frequency and type of editing reflected in mRNA isolated from a population of primary human hepatocytes (PHHs) edited with SpCas9 gRNA pairs delivered by LNP and targeting the indicated combinations of DNA target sequences, as assessed by RNA seq as described in Example 9.

FIG. 23B is a graph demonstrating the fold change in total LDLR protein levels in primary human hepatocytes (PHHs) treated with varying doses of SpCas9 gRNA pairs delivered by LNP and targeting the indicated combinations of DNA target sequences, as assessed by ELISA as described in Example 9.

FIG. 24 is a chart demonstrating the fold change in total LDLR protein levels in primary human hepatocytes (PHHs) edited with SpCas9 or AsCas12a gRNA pairs targeting the indicated combinations of DNA target sequences, assessed by ELISA as described in Example 10.

FIG. 25A is a graph demonstrating the baseline expression level of LDLR protein in HepG2 and 1C10 cells as described in Example 11.

FIG. 25B is a graph demonstrating the baseline LDL-C uptake by HepG2 and 1C10 cells as described in Example 11.

FIG. 26A is a graph demonstrating the expression level of LDLR protein in HepG2 and 1C10 cells after editing with SpCas9 gRNAs delivered by LNP as described in Example 11.

FIG. 26B is a graph demonstrating the LDL-C uptake by HepG2 and 1C10 cells after editing with SpCas9 gRNAs delivered by LNP as described in Example 11.

FIG. 27A is a graph demonstrating the frequency and type of edits in mouse subjects treated with dual SpCas9 or AsCas12a (WT) gRNAs delivered by LNP as assessed by UDiTaS, as described in Example 12. The indicated gRNA pair target sequences are described in Tables 29C-29D.

FIG. 27B is a graph demonstrating fold change in total Ldlr protein levels in mouse subjects treated with dual SpCas9 or AsCas12a (WT) gRNAs delivered by LNP as assessed by ELISA, as described in Example 12. The indicated gRNA pair target sequences are described in Tables 29C-29D.

FIG. 28A is a graph demonstrating the percent reduction in LDL-C levels in mouse subjects treated with dual SpCas9 or AsCas12a (WT) gRNAs delivered by LNP as assessed using an LDL-C assay kit (BioTechne) as described in Example 12. The indicated DNA target sequences are described in Tables 29C-29D.

FIG. 28B is a graph demonstrating the correlation of Ldlr fold upregulation (assessed by ELISA and depicted in FIG. 27B) and LDL-C percent reduction (assessed using an LDL-C assay kit (BioTechne) as depicted in FIG. 28A) in mouse subjects treated with dual SpCas9 or AsCas12a (WT) gRNAs delivered by LNP as described in Example 12. The indicated DNA target sequences are described in Tables 29C-29D.

FIG. 29A is a graph demonstrating dose-dependent effects of total RNA (i.e., the sum of mRNA and gRNA) on frequency and type of editing in mouse liver cells from mouse subjects treated with dual SpCas9 gRNAs targeting the Ldlr Proximal region delivered by LNP as assessed by RT-ddPCR, as described in Example 13. The indicated DNA target sequences are described in Table 31A.

FIG. 29B is a graph demonstrating dose-dependent effects of total RNA (i.e., the sum of mRNA and gRNA) on frequency and type of editing in mouse liver cells from mouse subjects treated with dual SpCas9 gRNAs targeting the Ldlr Proximal-ARE region delivered by LNP as assessed by RT-ddPCR, as described in Example 13. The indicated DNA target sequences are described in Table 31A.

FIG. 30A is a graph demonstrating dose-dependent effects of total RNA (i.e., the sum of mRNA and gRNA) on frequency and type of editing in mouse liver cells from mouse subjects treated with dual SpCas9 gRNAs targeting the Ldlr Proximal region delivered by LNP as assessed by Next-Generation Sequencing (NGS), as described in Example 13. The indicated DNA target sequences are described in Table 31A.

FIG. 30B is a graph demonstrating dose-dependent effects of total RNA (i.e., the sum of mRNA and gRNA) on frequency and type of editing in mouse liver cells from mouse subjects treated with dual SpCas9 gRNAs targeting the Ldlr Proximal-ARE region delivered by LNP as assessed by Next-Generation Sequencing (NGS), as described in Example 13. The indicated DNA target sequences are described in Table 31A.

FIG. 31A is a graph demonstrating fold change in liver Ldlr protein levels in mouse subjects treated with dual SpCas9 gRNAs targeting the Proximal region delivered by LNP as assessed by ELISA, as described in Example 13. The indicated DNA target sequences are described in Table 31A.

FIG. 31B is a graph demonstrating fold change in liver Ldlr protein levels in mouse subjects treated with dual SpCas9 gRNAs targeting the Proximal-ARE region delivered by LNP as assessed by ELISA, as described in Example 13. The indicated DNA target sequences are described in Table 31A.

FIG. 32A is a graph demonstrating change in serum LDL-C levels in mouse subjects treated with dual SpCas9 gRNAs targeting the Ldlr Proximal region delivered by LNP as described in Example 13. The indicated DNA target sequences are described in Table 31A.

FIG. 32B is a graph demonstrating change in serum LDL-C levels in mouse subjects treated with dual SpCas9 gRNAs targeting the Ldlr Proximal-ARE region delivered by LNP as described in Example 13. The indicated DNA target sequences are described in Table 31A.

FIG. 33A is a graph demonstrating change in serum ApoB levels in mouse subjects treated with dual SpCas9 gRNAs targeting the Ldlr Proximal region delivered by LNP as described in Example 13. The indicated DNA target sequences are described in Table 31A.

FIG. 33B is a graph demonstrating change in serum ApoB levels in mouse subjects treated with dual SpCas9 gRNAs targeting the Ldlr Proximal-ARE region delivered by LNP as described in Example 13. The indicated DNA target sequences are described in Table 31A.

FIG. 34A is a graph demonstrating the percent change in serum ApoB levels in wild-type mouse subjects treated with dual SpCas9 gRNAs targeting the Ldlr Proximal region delivered by LNP as described in Example 13. The indicated DNA target sequences are described in Table 31A.

FIG. 34B is a graph demonstrating the percent change in serum ApoB levels in wild-type mouse subjects treated with dual SpCas9 gRNAs targeting the Ldlr Proximal-ARE region delivered by LNP as described in Example 13. The indicated DNA target sequences are described in Table 31A.

FIG. 34C is a graph demonstrating the correlation frequency of edits (assessed by NGS and depicted in FIG. 30B) of Ldlr fold upregulation (assessed by ELISA and depicted in FIG. 31B) and ApoB percent reduction (as depicted in FIG. 33B) in wild-type mouse subjects treated with dual SpCas9 gRNAs selected to induce the Proximal-ARE deletion delivered by LNP, as described in Example 13. The indicated DNA target sequences are described in Table 31A.

FIG. 35A is a graph demonstrating the change in Ldlr mRNA levels in response to varying doses of SpCas9 gRNA target sequences delivered by LNP in wild-type mouse subjects as described in Example 14.

FIG. 35B is a graph demonstrating the change in serum ApoB levels in response to varying doses of SpCas9 gRNA pairs delivered by LNP in wild-type mouse subjects as described in Example 14.

FIG. 36A is a graph demonstrating the frequency of functional edits in response to varying doses of SpCas9 gRNA pairs delivered by LNP in wild-type or Ldlr^{-/-} mouse subjects as described in Example 14.

FIG. 36B is a graph demonstrating the change in LDLR protein levels in response to varying doses of SpCas9 gRNA pairs delivered by LNP in wild-type or Ldlr/mouse subjects as described in Example 14.

FIG. 36C is a graph demonstrating the change in serum ApoB levels in response to varying doses of SpCas9 gRNA pairs delivered by LNP in wild-type or Ldlr/mouse subjects as described in Example 14.

FIG. 37A is a graph demonstrating the correlation frequency of edits (assessed by NGS and depicted in FIG. 36A) of Ldlr fold upregulation (assessed by ELISA and depicted in FIG. 36B) and ApoB percent reduction (as depicted in FIG. 36C) in Ldlr/+ mouse subjects treated with dual SpCas9 gRNAs selected to induce the Proximal-ARE deletion delivered by LNP as described in Example 14.

FIG. 37B is a graph demonstrating change in serum LDL-C levels in Ldlr-/+ mouse subjects treated with dual SpCas9 gRNAs targeting the Ldlr Proximal-ARE region delivered by LNP as measured by enzymatic LDL-C assay as described in Example 14.

FIG. 37C is a graph demonstrating the change in serum LDL-C levels in Ldlr^{+/-} mouse subjects treated with dual SpCas9 gRNAs targeting the Ldlr Proximal region delivered by LNP as measured an HPLC-based quantification method as described in Example 14.

FIG. 38A is an experimental design diagram for the experiment evaluating LDL-C levels in mouse subjects fed a regular or high-fat diet as described in Example 15.

FIG. 38B is a graph depicting absolute serum LDL-C concentrations in wild-type mice fed a regular or high-fat diet and treated with dual SpCas9 gRNAs targeting the Ldlr Proximal-ARE region delivered by LNP as described in Example 15.

FIG. 38C is a graph depicting the percent change in serum LDL-C levels in wild-type mice subjects fed a regular or high-fat diet and treated with dual SpCas9 gRNAs targeting the Ldlr Proximal-ARE region delivered by LNP at 11 weeks, as described in Example 15.

FIG. 38D is a graph depicting the absolute change in serum LDL-C levels in wild-type and Ldlr^{+/-} mice subjects fed a high-fat diet and treated with dual SpCas9 gRNAs targeting the Ldlr Proximal-ARE region delivered by LNP at 11 weeks, as described in Example 15.

FIG. 38E is a graph demonstrating the change in LDLR protein levels in wild-type and Ldlr^{-/-} mouse subjects fed a high-fat diet and treated with dual SpCas9 gRNAs targeting the Ldlr Proximal-ARE region delivered by LNP as described in Example 15.

FIG. 39 is a graph depicting mRNA fold upregulation and the frequency and type of editing in a population of cynomolgus hepatocytes edited with dual SpCas9 or AsCas12a (WT) gRNAs delivered by LNP as assessed by RT-ddPCR, as described in Example 16. The indicated DNA target sequences are described in Table 33A.

FIG. 40 is a graph demonstrating the fold change in total LDLR protein levels in cynomolgus hepatocytes edited with dual SpCas9 or AsCas12a gRNAs assessed by ELISA, as described in Example 16.

FIG. 41 is a study diagram illustrating the experiments and results identifying off-target editing of SpCas9 gRNAs as described in Example 17.

FIG. 42A is a graph demonstrating the fold change in LDLR protein levels in Heterozygous Familial Hypercholesterolemia (HeFH) patient-derived fibroblasts edited with the Proximal-ARE SpCas9 gRNA pair, delivered by LNP, and assessed by ELISA as described in Example 18.

FIG. 42B is a graph demonstrating the fold change in LDL-C uptake by Heterozygous Familial Hypercholesterolemia (HeFH) patient-derived fibroblasts edited with the Proximal-ARE SpCas9 gRNA pair, delivered by LNP, and assessed by flow cytometry as described in Example 18.

FIG. 43A is a graph demonstrating the normalized percent serum LDL-C remaining, relative to baseline, at multiple time points after administration of the SpCas9 Proximal-ARE gRNA pair delivered by LNP to cynomolgus monkey subjects as described in Example 19.

FIG. 43B is a graph demonstrating the normalized percent serum LDL-C remaining, relative to baseline, at multiple time points after administration of the SpCas9 Proximal-ARE gRNA pair delivered by LNP to cynomolgus monkey subjects as described in Example 19.

FIG. 43C is a graph demonstrating the correlation of functional editing and LDL-C percent reduction (as depicted in FIG. 43A) in cynomolgus monkey subjects treated with dual SpCas9 gRNAs delivered by LNP as described in Example 19.

FIG. 43D is a graph demonstrating the correlation of liver LDLR protein levels and LDL-C percent reduction (as depicted in FIG. 43A) in cynomolgus monkey subjects treated with dual SpCas9 gRNAs delivered by LNP as described in Example 19.

FIG. 43E is a graph depicting editing levels in liver of NHPs. Excisions, inversions, small indels, large resections and translocations in liver specimens of NHPs treated with a single IV dose at the indicated multiple dose levels as described in Example 19. Bars indicate total % editing and individual sub-bars the specific type of edit. NHP=3 at 1.5; 2 at 2 mg/kg; 2 at 2 mg/kg and 3 at 4 mg/kg; ±SD.

FIG. 43F is a graph depicting LDLR protein levels in NHPs. LDLR protein levels are increased in the livers of NHPs treated as described in Example 19 in a dose-responsive manner. Bars indicate mean values at each dose levels, NHP=3 at 1.5; 2 at 2 mg/kg; 2 at 2 mg/kg and 3 at 4 mg/kg; ±SD. Dotted line indicates mean of vehicle-treated animals (NHP=3).

FIG. 43G is a graph depicting ApoB reduction in NHPs as described in Example 19. Single IV administration at the indicated dose levels showed significant reduction of plasma ApoB, expressed as change from baseline levels of individual animals. NHP=3 at 1.5; 2 at 2 mg/kg; 2 at 3 mg/kg and 3 at 4 mg/kg; ±SD.

FIG. 44A is a micrograph demonstrating staining for Cas9 mRNA (red) and nuclei (blue) in a liver sample from a cynomolgus monkey subject treated with dual SpCas9 gRNAs delivered by LNP as described in Example 19.

FIG. 44B is a micrograph demonstrating staining for Cas9 mRNA (red) and nuclei (blue) in an ovary sample from a cynomolgus monkey subject treated with dual SpCas9 gRNAs delivered by LNP as described in Example 19.

FIG. 45A is a micrograph demonstrating staining for Cas9 mRNA (red) and nuclei (blue) in a liver sample from a cynomolgus monkey subject after about 24 hours of treatment with dual SpCas9 gRNAs delivered by LNP as described in Example 19.

FIG. 45B is a micrograph demonstrating staining for Cas9 protein (brown) and nuclei (blue) in a liver sample from a cynomolgus monkey subject after about 24 hours of treatment with dual SpCas9 gRNAs delivered by LNP as described in Example 19.

FIG. 45C is a micrograph demonstrating staining for Cas9 mRNA (red) and nuclei (blue) in an ovary sample from a cynomolgus monkey subject after about 24 hours of treatment with dual SpCas9 gRNAs delivered by LNP as described in Example 19.

FIG. 45D is a micrograph demonstrating staining for Cas9 protein (brown) and nuclei (blue) in an ovary sample from a cynomolgus monkey subject after about 24 hours of treatment with dual SpCas9 gRNAs delivered by LNP as described in Example 19.

FIG. 45E is a micrograph demonstrating staining for LDLR protein (brown) and nuclei (blue) in a liver sample from a cynomolgus monkey subject after about 24 hours of treatment with dual SpCas9 gRNAs delivered by LNP as described in Example 19.

FIG. 46 is a graph demonstrating the percent of indels generated (assessed using UDiTaS) in multiple tissue samples collected from a cynomolgus monkey (non-human primate) subject after about 24 hours of treatment with dual SpCas9 gRNAs delivered by LNP as described in Example 19.

FIG. 47A is a graph demonstrating the percent Lp(a) protein reduction using an immunoturbidimetry assay relative to baseline, at multiple time points (pre-dose, pre-dose, 24 hours, 48 hours, day 5, day 8, day 15, day 22, and day 29) after administration of the SpCas9 Proximal-ARE gRNA pair delivered by LNP (doses of 1.5 mpk, 2 mpk, 3 mpk, and 4 mpk) to cynomolgus monkey subjects as described in Example 19.

FIG. 47B is a graph demonstrating the percent Lp(a) protein reduction using an immunoturbidimetry assay (pre-dose averaged), at multiple time points (pre-dose, pre-dose, 24 hours, 48 hours, day 5, day 8, day 15, day 22, and day 29) after administration of the SpCas9 Proximal-ARE gRNA pair delivered by LNP (doses of 1.5 mpk, 2 mpk, 3 mpk, and 4 mpk) to cynomolgus monkey subjects as described in Example 19.

### DETAILED DESCRIPTION

The present disclosure relates to genome editing systems (e.g., RNA-guided nuclease-related, e.g., CRISPR/Cas-related, genome editing systems), compositions, delivery vehicles, and methods for targeting an LDLR nucleic acid sequence, editing a target LDLR nucleic acid sequence, or modulating expression of an LDLR gene, and applications thereof. The present disclosure also provides genome editing systems, compositions, vectors, and methods for editing cells using CRISPR/Cas-related components to edit a target LDLR gene. The present disclosure also provides lipid nanoparticle (LNP) facilitated delivery of genome editing systems and methods for editing cells using CRISPR/Cas-related components delivered via LNP to edit a target LDLR gene. Some aspects of the present disclosure provide pharmaceutical compositions, cells, cell populations, methods, strategies, and treatment modalities that are useful in the context of treating and/or managing a disease, e.g., hyperlipidemia or hypercholesterolemia.

The subject matter of the present disclosure is described with reference to the Figures. It should be understood that numerous specific details, relationships, and methods are set forth in this Detailed Description, Examples, and accompanying Figures to provide a more complete understanding of the subject matter disclosed herein. For purposes of clarity of disclosure and not by way of limitation, the Detailed Description is divided into the following subsections:
1. Definitions and Abbreviations
2. Genome editing systems
3. Guide RNA (gRNA) molecules
4. Guide RNA design
5. RNA-guided nucleases
6. Genome editing strategies
7. Implementation of genome editing systems: delivery, formulations, and routes of administration
8. Examples

### 1. Definitions and Abbreviations

Unless otherwise specified, each of the following terms has the meaning associated with it in this section.

The indefinite articles “a” and “an” refer to at least one of the associated noun and are used interchangeably with the terms “at least one” and “one or more.” For example, “a module” means at least one module, or one or more modules.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The conjunctions “or” and “and/or” are used interchangeably as non-exclusive disjunctions.

The phrase “consisting essentially of” means that the species recited are the predominant species, but that other species can be present in trace amounts or amounts that do not affect structure, function or behavior of the subject composition. For instance, a composition that consists essentially of a particular species will generally comprise 90%, 95%, 96%, or more of that species.

“Domain” is used to describe a segment of a protein or nucleic acid. Unless otherwise indicated, a domain is not required to have any specific functional property.

An “indel” is an insertion and/or deletion in a nucleic acid sequence. An indel can be the product of the repair of a DNA double strand break, such as a double strand break formed by a genome editing system of the present disclosure. An indel is most commonly formed when a break is repaired by an “error prone” repair pathway such as the non-homologous end joining (NHEJ) pathway described below.

“Gene conversion” refers to the alteration of a DNA sequence by incorporation of an endogenous homologous sequence (e.g., a homologous sequence within a gene array).

“Gene correction” refers to the alteration of a DNA sequence by incorporation of an exogenous homologous sequence, such as an exogenous single- or double stranded donor template DNA. Gene conversion and gene correction are products of the repair of DNA double-strand breaks by HDR pathways such as those described below.

Indels, gene conversion, gene correction, and other genome editing outcomes are typically assessed by sequencing (most commonly by “next-gen” or “sequencing-by-synthesis” methods, though Sanger sequencing can still be used) and are quantified by the relative frequency of numerical changes (e.g., +1, +2 or more bases) at a site of interest among all sequencing reads. DNA samples for sequencing can be prepared by a variety of methods known in the art and can involve the amplification of sites of interest by polymerase chain reaction (PCR), the capture of DNA ends generated by double strand breaks, as in the GUIDE-seq process described in Tsai et al. (Nat. Biotechnol. 34 (5): 483 (2016), incorporated by reference herein), the UDiTaS method described in Giannoukos et al. (BMC Genomics. 19 (1): 212 (2018), incorporated by reference herein), or by other means well known in the art. Genome editing outcomes can also be assessed by in situ hybridization methods such as the FiberComb™ system commercialized by Genomic Vision (Bagneux, France), and by any other suitable methods known in the art.

“Alt-HDR,” “alternative homology-directed repair,” or “alternative HDR” are used interchangeably to refer to the process of repairing DNA damage using a homologous nucleic acid (e.g., an endogenous homologous sequence, e.g., a sister chromatid, or an exogenous nucleic acid, e.g., a template nucleic acid). Alt-HDR is distinct from canonical HDR in that the process utilizes different pathways from canonical HDR, and can be inhibited by the canonical HDR mediators, RAD51 and BRCA2. Alt-HDR is also distinguished by the involvement of a single-stranded or nicked homologous nucleic acid template, whereas canonical HDR generally involves a double-stranded homologous template.

“Canonical HDR,” “canonical homology-directed repair” or “cHDR” refer to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single stranded portion of DNA. In a normal cell, cHDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2, and the homologous nucleic acid is typically double stranded.

Unless indicated otherwise, the term “HDR” as used herein encompasses both canonical HDR and alt-HDR.

“Non-homologous end joining” or “NHEJ” refers to ligation mediated repair and/or non-template mediated repair including canonical NHEJ (cNHEJ) and alternative NHEJ (altNHEJ), which in turn includes microhomology-mediated end joining (MMEJ), single-strand annealing (SSA), and synthesis-dependent microhomology-mediated end joining (SD-MMEJ).

“Replacement” or “replaced,” when used with reference to a modification of a molecule (e.g., a nucleic acid or protein), does not require a process limitation but merely indicates that the replacement entity is present.

“Knock-out” or “knockout” refers to an inactivating mutation in a target gene, wherein the product of the target gene comprises a loss of function.

As used herein, “upregulate” refers to an increase in expression and/or abundance of a target gene product, for example by mutation of a target gene.

“Gain of function” refers to a mutation in a target gene, wherein the protein product of the gene has an increase in a functional activity. This may be achieved, e.g. through an increase in abundance and/or a change in the capacity of the protein product to carry out a function.

“Gene product” refers to biochemical products resulting from the expression of the gene and includes the RNA or protein that is encoded by the gene.

As used herein, “expression” refers to the amount of gene product (e.g., LDLR) produced through the transcription and/or translation of a gene.

“On-target site” refers to the exact genomic sequence or locus within a gene of interest for which a guide RNA/RNA-guided nuclease was designed to target. “Off-target site” refers to a genomic sequence or locus that is not the on-target site and may be (or is found to be) edited by the guide RNA/RNA guided nuclease.

As used herein, the term “hairpin” or “hairpin region” refers to a nucleic acid secondary structure comprising a stem-loop structure. A guide-RNA of the present disclosure may comprise one or more hairpins, or hairpin regions, that are not part of its targeting domain.

As used herein, the term “pseudoknot” refers to a nucleic acid secondary structure comprising at least two stem-loop structures in which half of one stem is intercalated between the two halves of another stem. Exemplary pseudoknot sequences are bolded in the two gRNA sequences shown below;

[SEQ ID NO: 24]
rUrArArUrUrUrCrUrArCrUrCrUrUrGrUrArGrArUrGrG
rCrCrUrGrArGrArUrGrCrCrArGrCrUrGrUrCrCrUrArA
rUrUrUrCrUrArCrUrCrUrUrGrUrArGrArU;
[SEQ ID NO: 25]
mU*rArArUrUrUrCrUrArCrUrCrUrUrGrUrArGrArUrG
rGrCrCrUrGrArGrArUrGrCrCrArGrCrUrGrUrCrCrUrA
rArUrUrUrCrUrArCrUrCrUrUrGrUrArGrA*mU.

As used herein, the term “locked nucleic acid” or “LNA” refers to a modified RNA nucleotide in which the ribose moiety is modified with a bridge connecting the 2′ oxygen and 4′ carbon. An exemplary nucleic acid including a LNA is shown below, where the “+” indicates a locked nucleotide;

[SEQ ID NO: 26]
/5IdT/+A+T+G+T+G+T+T+T+T+T+G+T+C+A+A+A+A+G+A+
C+C+T+T+T+TrUrArArUrUrUrCrUrArCrUrCrUrUrGrUrA
rGrArUrCrArArCrCrUrCrCrUrGrGrCrCrArGrArUrUrC
rUrC/3InvdT/.

Subject” means a human or non-human animal. A human subject can be any age (e.g., an infant, child, young adult, or adult), and can suffer from a disease, or can be in need of alteration of a gene. Alternatively, the subject can be an animal, which term includes, but is not limited to, mammals, birds, fish, reptiles, amphibians, and more particularly non-human primates (NHP), rodents (such as mice, rats, hamsters, etc.), rabbits, guinea pigs, dogs, cats, and so on. In certain embodiments of this disclosure, the subject is livestock, e.g., a cow, a horse, a sheep, or a goat. In certain embodiments, the subject is poultry.

As used herein a “therapeutically effective amount” refers to the amount of a cell and/or composition that when administered to a subject for treating a disease, is sufficient to beneficially affect such treatment for the disease.

“Treat,” “treating,” and “treatment” mean the treatment of a disease in a subject (e.g., a human subject), including one or more of inhibiting the disease, i.e., arresting or preventing its development or progression; relieving the disease, i.e., causing regression of the disease state; relieving one or more symptoms of the disease; and curing the disease.

“Prevent,” “preventing,” and “prevention” refer to the prevention of a disease in a mammal, e.g., in a human, including (a) avoiding or precluding the disease; (b) affecting the predisposition toward the disease; or (c) preventing or delaying the onset of at least one symptom of the disease.

A “kit” refers to any collection of two or more components that together constitute a functional unit that can be employed for a specific purpose. By way of illustration (and not limitation), one kit according to this disclosure can include a guide RNA complexed or able to complex with an RNA-guided nuclease, and accompanied by (e.g., suspended in, or suspendable in) a pharmaceutically-acceptable carrier. The kit can be used to introduce the complex into, for example, a cell or a subject, for the purpose of causing a desired genomic alteration in such cell or subject. The components of a kit can be packaged together, or they can be separately packaged. Kits according to this disclosure also optionally include directions for use (DFU) that describe the use of the kit e.g., according to a method of this disclosure. The DFU can be physically packaged with the kit, or it can be made available to a user of the kit, for instance by electronic means.

The terms “polynucleotide”, “nucleotide sequence”, “nucleic acid”, “nucleic acid molecule”, “nucleic acid sequence”, and “oligonucleotide” mean any chain of two or more nucleotide bases (also called “nucleotides”). The polynucleotides, nucleotide sequences, nucleic acids etc. can be chimeric mixtures or derivatives or modified versions thereof, single-stranded or double-stranded. They can be modified at the base moiety, sugar moiety, or phosphate backbone, for example, to improve stability of the molecule, its hybridization parameters, etc. A nucleotide sequence typically carries genetic information, including, but not limited to, the information used by cellular machinery to make proteins and enzymes. These terms include double- or single-stranded genomic DNA, RNA, any synthetic and genetically manipulated polynucleotide, and both sense and antisense polynucleotides. These terms also include nucleic acids containing modified bases.

Conventional International Union of Pure and Applied Chemistry (IUPAC) notation is used in nucleotide sequences presented herein, as shown in Table 1, below (see also Cornish-Bowden A, Nucleic Acids Res. 1985 May 10; 13 (9): 3021-30, incorporated by reference herein). It should be noted, however, that “T” denotes “Thymine or Uracil” in those instances where a sequence can be encoded by either DNA or RNA, for example in a gRNA, for example in a gRNA targeting domain. The prefix r (for ribo) may be used for emphasis or clarity.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “protein,” “peptide” and “polypeptide” are used interchangeably to refer to a sequential chain of amino acids linked together via peptide bonds. The terms include individual proteins, groups or complexes of proteins that associate together, as well as fragments or portions, variants, derivatives and analogs of such proteins. Peptide sequences are presented herein using conventional notation, beginning with the amino or N-terminus on the left, and proceeding to the carboxyl or C-terminus on the right. Standard one-letter or three-letter abbreviations can be used.

The term “variant” refers to an entity such as a polypeptide, polynucleotide or small molecule that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity.

As used herein, the term “promoter” refers to a region (i.e., a DNA sequence) of a genome that initiates the transcription of a gene.

As used herein, the term “3′ untranslated region” or “3′ UTR” refers to a region of a gene that is transcribed, does not form part of the protein-coding portion of a messenger RNA (mRNA), and is located to the 3′ of the protein-coding portion of the messenger RNA (mRNA), i.e. after the stop codon.

The term “endogenous,” as used herein in the context of nucleic acids (e.g., genes, protein-encoding genomic regions, promoters) or proteins, refers to a native nucleic acid or protein in its natural location, e.g., within the genome of a cell. In contrast, the term “exogenous,” as used herein in the context of nucleic acids, e.g., expression constructs, cDNAs, indels, and nucleic acid vectors, or proteins, refers to nucleic acids or proteins that have artificially been introduced into the cell. For example, an exogenous nucleic acid may be introduced into the genome of a cell using, for example, gene-editing or genetic engineering techniques, e.g., CRISPR-based editing techniques.

The terms “RNA-guided nuclease” and “RNA-guided nuclease molecule” are used interchangeably herein. In some embodiments, the RNA-guided nuclease is an RNA-guided DNA endonuclease enzyme. In some embodiments, the RNA-guided nuclease is a CRISPR nuclease. Non-limiting examples of RNA-guided nucleases are listed in Table 2 below, and the methods and compositions disclosed herein can use any combination of RNA-guided nucleases disclosed herein, or known to those of ordinary skill in the art. Those of ordinary skill in the art will be aware of additional nucleases and nuclease variants suitable for use in the context of the present disclosure, and it will be understood that the present disclosure is not limited in this respect.

TABLE 2
Exemplary RNA-Guided Nucleases

| | Length | | |
|---|---|---|---|
| Nuclease | (amino acid) | PAM | Reference |
| SpCas9 | 1368 | NGG | Cong et al., Science. 2013; 339(6121): 819-23 |
| SaCas9 | 1053 | NNGRRT | Ran et al., Nature. 2015; 520(7546): 186-91. |
| (KKH) | 1067 | NNNRRT | Kleinstiver et al., Nat Biotechnol. 2015; 33(12): 1293- |
| SaCas9 | | | 1298 |
| AsCas12a | 1353 | TTTV | Zetsche et al., Nat Biotechnol. 2017; 35(1): 31-34. |
| (AsCpf1) | | | |
| LbCas12a | 1274 | TTTV | Zetsche et al., Cell. 2015; 163(3): 759-71. |
| (LbCpf1) | | | |
| CasX | 980 | TTC | Burstein et al., Nature. 2017; 542(7640): 237-241. |
| CasY | 1200 | TA | Burstein et al., Nature. 2017; 542(7640): 237-241. |
| Cas12hl | 870 | RTR | Yan et al., Science. 2019; 363(6422): 88-91. |
| Cas12il | 1093 | TTN | Yan et al., Science. 2019; 363(6422): 88-91. |
| Cas12cl | unknown | TG | Yan et al., Science. 2019; 363(6422): 88-91. |
| Cas12c2 | unknown | TN | Yan et al., Science. 2019; 363(6422): 88-91. |
| eSpCas9 | 1423 | NGG | Chen et al., Nature. 2017; 550(7676): 407-410. |
| Cas9-HF1 | 1367 | NGG | Chen et al., Nature. 2017; 550(7676): 407-410. |
| HypaCas9 | 1404 | NGG | Chen et al., Nature. 2017; 550(7676): 407-410. |
| dCas9- | 1623 | NGG | U.S. Patent No. 9,322,037 |
| Fokl | | | |
| Sniper- | 1389 | NGG | Lee et al., Nat Commun. 2018; 9(1): 3048. |
| Cas9 | | | |
| xCas9 | 1786 | NGG, NG, | Wang et al., Plant Biotechnol J. 2018; pbi. 13053. |
| | | GAA, GAT | |
| AaCas12b | 1129 | TTN | Teng et al. Cell Discov. 2018; 4: 63. |
| evoCas9 | 1423 | NGG | Casini et al., Nat Biotechnol. 2018; 36(3): 265-271. |
| SpCas9- | 1423 | NG | Nishimasu et al., Science. 2018; 361(6408): 1259- |
| NG | | | 1262. |
| VRQR | 1368 | NGA | Li et al., The CRISPR Journal, 2018; 01: 01 |
| VRER | 1372 | NGCG | Kleinstiver et al., Nature. 2016; 529(7587): 490-5. |
| NmeCas9 | 1082 | NNNGATT | Amrani et al., Genome Biol. 2018; 19(1): 214. |
| CjCas9 | 984 | NNNNRYA | Kim et al., Nat Commun. 2017; 8: 14500. |
| BhCas12b | 1108 | ATTN | Strecker et al., Nat Commun. 2019 January |
| | | | 22; 10(1): 212. |
| BhCas12b | 1108 | ATTN | Strecker et al., Nat Commun. 2019 January |
| V4 | | | 22; 10(1): 212. |
| CasΦ | | | Pausch et al., Science 2020; 369(6501): 333-337. |

Additional suitable RNA-guided nucleases, e.g., Cas9 and Cas12 nucleases, will be apparent to the skilled artisan in view of the present disclosure, and the disclosure is not limited by the exemplary suitable nucleases provided herein. In some embodiment, a suitable nuclease is a Cas9 or Cas12a (Cpf1) nuclease. In some embodiments, the disclosure also embraces nuclease variants, e.g., Cas9 or Cas12a nuclease variants. A nuclease variant refers to a nuclease comprising an amino acid sequence characterized by one or more amino acid substitutions, deletions, or additions as compared to the wild type (WT) amino acid sequence of the nuclease. As used herein, AsCas12a (WT) generally refers to the M537R, H800A, and F870L triple mutant (e.g., SEQ ID NO: 1348, but could also refer to ‘true’ wild-type (e.g., Uniprot Identifier U2UMQ6). Suitable nucleases and nuclease variants may also comprise purification tags (e.g., polyhistidine tags) and signaling peptides, e.g., comprising or consisting of a nuclear localization signal sequence (NLS). Some non-limiting examples of suitable nucleases and nuclease variants are described in more detail elsewhere herein, and also comprise those described in PCT application PCT/US2019/22374, filed Mar. 14, 2019, and entitled “Systems and Methods for the Treatment of Hemoglobinopathies,” the entire contents of which are incorporated herein by reference.

In some embodiments, the RNA-guided nuclease is a Streptococcus pyogenes Cas9 (SpCas9). In some embodiments, the RNA-guided nuclease is an SpCas9 variant. In some embodiments, the RNA-guided nuclease is an Acidaminococcus sp. Cas12a (AsCas12a or AsCpf1). In some embodiments, the RNA-guided nuclease is an AsCas12a variant. Suitable nuclease variants, including suitable SpCas9 or AsCas12a variants will be known or apparent to those of ordinary skill in the art based on the present disclosure, and include, but are not limited to, the SpCas9 or AsCas12a variants disclosed herein or otherwise known in the art. For example, in some embodiments, the RNA-guided nuclease is an Acidaminococcus sp. Cas12a RR variant (AsCas12a-RR). In certain embodiments, the RNA-guided nuclease is a Cas12a RVR variant. These and other variants are described in PCT patent application PCT/US2017/028420 and Guo et al., Nat Biotechnol. 2017 August; 35 (8): 789-792, each of which is incorporated by reference herein for all purposes. Exemplary SpCas9 variants comprising mutations corresponding to the D10A mutation are depicted in SEQ ID NOs 1341, 1343, and 1346. Exemplary SpCas9 variants comprising mutations corresponding to the D10A/H840A mutation are depicted in SEQ ID NOs 1342, 1344, and 1347. Exemplary AsCas 12a RR variants (comprising mutations corresponding to the S542R/K607R mutations) are depicted in SEQ ID NOs: 50 and 51. Exemplary AsCas12a RVR variants (comprising mutations corresponding to the S542R/K548V/N552R mutations) are depicted in SEQ ID NOs: 52 and 53. Additionally, or alternatively, suitable Cas12a variants include those having an M537R substitution, an H800A substitution, and/or an F870L substitution, or any combination thereof (numbering scheme according to AsCas12a wild-type sequence).

In certain embodiments, the RNA-guided nucleases of the present disclosure can comprise a DNA modifying enzyme for targeted nucleotide alteration, commonly referred to as a “base editor.” In certain embodiments, the base editor is a cytosine base editor. In certain embodiments, the base editor is an adenosine base editor.

In certain embodiments, the RNA-guided nucleases of the present disclosure can comprise a DNA modifying enzyme fused to a reverse transcriptase for targeted nucleotide insertion, deletion, or substitution, commonly referred to as a “prime editor.”

The term “targeting moiety” or “targeting modification” refers to any molecule that provides an enhanced affinity for a selected target, e.g., a cell, cell type, tissue, organ, region of the body, or a compartment, e.g., a cellular, tissue or organ compartment. Some exemplary targeting moieties include, but are not limited to, antibodies, antigens, carbohydrate base moieties, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands. Carbohydrate based targeting moieties include, but are not limited to, D-galactose, multivalent galactose, N-acetyl-D-galactosamine (GalNAc), multivalent GalNAc, e.g., GalNAc2 and GalNAc3; D-mannose, multivalent mannose, multivalent lactose, N-acetyl-glucosamine, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

The terms “low-density lipoprotein receptor” and “LDLR” refer to gene ENSG00000130164 on Chromosome 19 of the Homo sapiens genome, or its accepted homologues in an animal species, i.e. animal as defined under “subject” above. These terms also refer to the protein coding product of this gene. LDLR (italicized) is used to denote nucleic acid (e.g. the chromosomal gene or an mRNA transcript thereof) and LDLR (not italicized) is used to denote the protein product of the gene.

The term “Icelandic mutation” refers to a 2.5 kb deletion in the 3′ UTR of the LDLR gene characterized in an Icelandic family (Bjornsson et al. Circ Genom Precis Med. 2021 February; 14 (1): e003029 [Bjornsson], incorporated by reference herein).

As used herein, the term “functional edit” or “functional editing” refers to the type of molecular edit events required for LDLR upregulation, for example, excision (or deletion) and inversion events.

### 2. Genome Editing Systems

Various genome editing systems known in the art can be used for the methods disclosed herein. Non-limiting examples of genome editing systems that can be used with the presently-disclosed subject matter include, but are not limited to CRISPR systems, zinc-finger nuclease (ZFN) systems, transcription activator-like effector nuclease (TALEN) systems, meganuclease (MN) systems, MegaTAL systems, other targeted endonuclease systems, and other chimeric endonuclease systems.

In certain embodiments, the genome editing system has RNA-guided DNA editing activity. In certain embodiments, the genome editing system includes at least two components adapted from naturally-occurring CRISPR systems: a guide RNA (gRNA) and an RNA-guided nuclease. These two components form a complex that is capable of associating with a specific nucleic acid sequence and optionally editing the DNA in or around that nucleic acid sequence, for instance by making one or more of a single-strand break (an SSB or nick), a double-strand break (a DSB) and/or a point mutation.

Naturally-occurring CRISPR systems are organized evolutionarily into two classes and five types (Makarova et al. Nat Rev Microbiol. 2011 June; 9 (6): 467-477 [Makarova], incorporated by reference herein), and while genome editing systems of the present disclosure can adapt components of any type or class of naturally-occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cas12a) and one or more guide RNAs (e.g., a crRNA and, optionally, a tracrRNA) that form ribonucleoprotein (RNP) complexes that associate with (i.e., target) and cleave specific loci complementary to a targeting (or spacer) sequence of the crRNA. Genome editing systems according to the present disclosure similarly target and optionally edit cellular DNA sequences but differ significantly from CRISPR systems occurring in nature. For example, the unimolecular guide RNAs described herein do not occur in nature, and both guide RNAs and RNA-guided nucleases according to this disclosure can incorporate any number of non-naturally occurring modifications.

Genome editing systems disclosed herein can be delivered into a cell by electroporation. Other non-viral approaches can also be employed for genome editing of target cells disclosed herein. For example, a nucleic acid molecule can be introduced into cells/subjects by administering the nucleic acid by lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Lipid nanoparticles (LNPs) or liposomes are also contemplated for delivery of nucleic acid molecules into a cell. In some embodiments, the genome editing systems disclosed herein are delivered in vivo to a subject by administration of lipid nanoparticles (LNPs) containing one or more components of the genome editing system.

Genome editing system disclosed herein can be delivered into subjects or cells using viral vectors, e.g., retroviral vectors, gamma-retroviral vectors or lentiviral vectors. Combinations of a retroviral vector and an appropriate packaging line are suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art. Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Genome editing systems can be implemented (e.g., administered or delivered to a cell or a subject) in a variety of ways, and different implementations can be suitable for distinct applications. For instance, a genome editing system is implemented, in certain embodiments, as a protein/RNA complex (a ribonucleoprotein complex, or RNP complex), which can be included in a pharmaceutical composition that optionally includes a pharmaceutically acceptable carrier and/or an encapsulating agent, such as a lipid or polymer micro- or nanoparticle, micelle, liposome, etc. In certain embodiments, a genome editing system is implemented as one or more nucleic acids encoding the RNA-guided nuclease and/or guide RNA components described above (optionally with one or more additional components). In certain embodiments, the genome editing system is implemented as one or more vectors comprising such nucleic acids, for instance a viral vector such as an adeno-associated virus. In certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence or can be targeted to- and capable of editing in parallel-two or more specific nucleotide sequences through the use of two or more guide RNAs. The use of multiple gRNAs is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (Maeder), which is incorporated by reference herein, describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i.e., flanking) the point mutation, and forms DSBs that flank the mutation. This, in turn, promotes deletion of the intervening sequence, including the mutation, thereby eliminating the cryptic splice site, and restoring normal gene function.

Genome editing systems can, in some instances, form double strand breaks that are repaired by cellular DNA double-strand break mechanisms such as NHEJ or HDR. These mechanisms are described throughout the literature, for example by Davis & Maizels, PNAS, 111 (10): E924-932, Mar. 11, 2014 (Davis) (describing Alt-HDR); Frit et al. DNA Repair 17 (2014) 81-97 (Frit) (describing Alt-NHEJ); and Iyama and Wilson III, DNA Repair (Amst.) 2013-Aug; 12 (8): 620-636 (Iyama) (describing canonical HDR and NHEJ pathways generally).

Where genome editing systems operate by forming DSBs, such systems optionally comprise one or more components that promote or facilitate a particular mode of double-strand break repair or a particular repair outcome. For instance, Cotta-Ramusino also describes genome editing systems in which a single stranded oligonucleotide “donor template” is added; the donor template is incorporated into a target region of cellular DNA that is cleaved by the genome editing system and can result in a change in the target sequence.

In certain embodiments, genome editing systems modify a target sequence, or modify expression of a gene in or near the target sequence, without causing double-strand breaks, e.g., by causing single-strand breaks or no cleavage (i.e., no strand breaks). For example, a genome editing system can comprise an RNA-guided nuclease fused to a functional domain that acts on DNA, thereby modifying the target sequence or its expression. As one example, an RNA-guided nuclease can be connected to (e.g., fused to) a cytidine deaminase functional domain, and can operate by generating targeted C-to-A substitutions. An RNA-guided nuclease can also, for example, be connected to (e.g. fused to) an adenosine deaminase functional domain. Exemplary nuclease/deaminase fusions are described in Komor et al. Nature 533, 420-424 (19 May 2016) (Komor) and Kantor et al., Int. J. Mol. Sci. 21 (17) 6240 (2020), which are hereby incorporated by reference in their entirety. Further non-limiting examples of suitable base editors, variants thereof, and strategies for preparing RNA-guided nucleases comprising the same are described in PCT applications: PCT/US2020/016664, filed Feb. 4, 2020; PCT/US2020/018192, filed Feb. 13, 2020; PCT/US2020/049975, field Sep. 9, 2020; PCT/US2022/012054, filed Jan. 11, 2022; and PCT/US2022/078655, filed Oct. 25, 2022, the entire contents of each of which are incorporated herein by reference.

Alternatively, a genome editing system can utilize a cleavage-inactivated (i.e., a “dead”) nuclease, such as a dead Cas9 (dCas9), and can operate by forming stable complexes on one or more targeted regions of cellular DNA, thereby recruiting other functional domains and/or interfering with functions involving the targeted region(s) including, without limitation, mRNA transcription, chromatin remodeling, among others.

In certain embodiments, the RNA-guided nucleases of the present disclosure can comprise a polymerase domain (e.g., a reverse transcriptase domain). In certain embodiments, the RNA-guided nuclease may use a gRNA with a primer binding sequence and/or a template for the polymerase domain.

In certain embodiments, the RNA-guided nuclease may be a prime editor (PE), where the PE is an RNA-guided nuclease with nickase activity that is fused to a reverse transcriptase domain. In certain embodiments, the PE may use a prime editing gRNA (pegRNA), where the pegRNA is a gRNA with a primer binding sequence (PBS) and a donor template, e.g., added at one of the termini, e.g., the 3′ end. In certain embodiments, a PE: pegRNA complex binds to the target DNA, and the nickase domain of the prime editor nicks only one strand, generating a flap. The PBS, located on the pegRNA, binds to the DNA flap and the edited RNA sequence is reverse transcribed using the reverse transcriptase domain of the prime editor. The edited strand is incorporated into the DNA at the end of the nicked flap, and the target DNA is repaired with the new reverse transcribed DNA. The original DNA segment is removed by a cellular endonuclease. Additional methods employing RNA-guided nucleases and polymerases for template mediated gene editing are described in PCT publications: WO 2020/191233, WO 2020/191248, WO 2021226558, WO2023283246, WO 2023/235501, and WO 2023/076898, each of which are incorporated by reference for all purposes herein.

3. Guide RNA (gRNA) Molecules

The terms “guide molecule,” “guide RNA” and “gRNA” refer to any nucleic acid that promotes the specific association (or “targeting”) of an RNA-guided nuclease such as a Cas9 or a Cas12a (Cpf1) to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al. (Molecular Cell 56 (2), 333-339, Oct. 23, 2014 (Briner), which is incorporated by reference), and in Cotta-Ramusino. The guide molecule can be an RNA molecule. The guide molecule can also comprise one or more nucleotides other than RNA nucleotides, for example, the guide molecule can be a DNA/RNA hybrid molecule, and/or the guide molecule can comprise one or more modified nucleotides (including, but not limited to, one or more modified DNA or RNA nucleotides).

In bacteria and archaea, type II CRISPR systems generally comprise an RNA-guided nuclease protein such as Cas9, a CRISPR RNA (crRNA) that comprises a 5′ region that is complementary to a foreign sequence, and a trans-activating crRNA (tracrRNA) that comprises a 5′ region that is complementary to, and forms a duplex with, a 3′ region of the crRNA. This duplex can facilitate the formation of- and is necessary for the activity of—the Cas/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g., GAAA) “tetraloop” or “linker” sequence bridging complementary regions of the crRNA (at its 3′ end) and the tracrRNA (at its 5′ end) (Mali et al. Science. 2013 Feb. 15; 339 (6121): 823-826 (Mali); Jiang et al. Nat Biotechnol. 2013 March; 31 (3): 233-239 (Jiang); and Jinek et al., 2012 Science August 17; 337 (6096): 816-821 (Jinek), all of which are incorporated by reference herein.)

Guide RNAs, whether unimolecular or modular, comprise a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013 September; 31 (9): 827-832, (Hsu), incorporated by reference herein), “complementarity regions” (Cotta-Ramusino), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cas12a gRNA.

In addition to the targeting domains, gRNAs typically (but not necessarily, as discussed below) comprise a plurality of domains that can influence the formation or activity of gRNA/Cas9 complexes. For instance, as mentioned above, the duplexed structure formed by first and secondary complementarity domains of a gRNA (also referred to as a repeat: anti-repeat duplex) interacts with the recognition (REC) lobe of Cas9 and can mediate the formation of Cas9/gRNA complexes. (Nishimasu et al., Cell 156, 935-949, Feb. 27, 2014 (Nishimasu 2014) and Nishimasu et al., Cell 162, 1113-1126 Aug. 27, 2015 (Nishimasu 2015), both incorporated by reference herein). It should be noted that the first and/or second complementarity domains can contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner, or A-U swaps. These and other similar modifications to the first and second complementarity domains are within the scope of the present disclosure.

Along with the first and second complementarity domains, Cas9 gRNAs typically comprise two or more additional duplexed regions that are involved in nuclease activity in vivo but not necessarily in vitro (Nishimasu 2015). A first stem-loop near the 3′ portion of the second complementarity domain is referred to variously as the “proximal domain” (Cotta-Ramusino), “stem loop 1” (Nishimasu 2014 and 2015), and the “nexus” (Briner). One or more additional stem loop structures are generally present near the 3′ end of the gRNA, with the number varying by species: S. pyogenes gRNAs typically comprise two 3′ stem loops (for a total of four stem loop structures including the repeat: anti-repeat duplex), while S. aureus and other species have only one (for a total of three stem loop structures). A description of conserved stem loop structures (and gRNA structures more generally) organized by species is provided in Briner.

While the foregoing description has focused on gRNAs for use with Cas9, it should be appreciated that other RNA-guided nucleases have been (or can in the future be) discovered or invented which utilize gRNAs that differ in some ways from those described to this point. For instance, Cas12a (also known as Cpf1; “CRISPR from Prevotella and Franciscella 1”) is an RNA-guided nuclease that does not require a tracrRNA to function. (Zetsche et al., 2015, Cell 163, 759-771 Oct. 22, 2015 (Zetsche I), incorporated by reference herein). A gRNA for use in a Cas12a genome editing system generally comprises a targeting domain and a complementarity domain (alternately referred to as a “handle”). It should also be noted that, in gRNAs for use with Cas12a, the targeting domain is usually present at or near the 3′ end, rather than the 5′ end as described above in connection with Cas9 gRNAs (the handle is at or near the 5′ end of a Cas12a gRNA).

Those of skill in the art will appreciate that, although structural differences can exist between gRNAs from different prokaryotic species, or between Cas12a and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that comprises one or more chemical modifications and/or sequential modifications (substitutions, additional nucleotides, truncations, etc.). Thus, for economy of presentation in this disclosure, gRNAs can be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular RNA-guided nuclease, e.g., a particular species of Cas9 or Cas12a. By way of illustration, the term gRNA can, in certain embodiments, comprise a gRNA for use with any RNA-guided nuclease occurring in a Class 2 CRISPR system, such as a type II or type V CRISPR system, or an RNA-guided nuclease derived or adapted therefrom.

In some embodiments, the guide RNA used comprises a modification as compared to the standard gRNA scaffold. Such modifications may comprise, for example, chemical modifications of a part of the gRNA, e.g., of a nucleobase or backbone moiety. In some embodiments, such a modification may also comprise the presence of one or more DNA nucleotide within the gRNA, e.g., within or outside of the targeting domain. In some embodiments, the modification may comprise an extension of the gRNA scaffold, e.g., by addition of 1-100 nucleotides, including RNA and/or DNA nucleotides at the 3′ or the 5′ terminus of the guide RNA, e.g., at the terminus distal to the targeting domain.

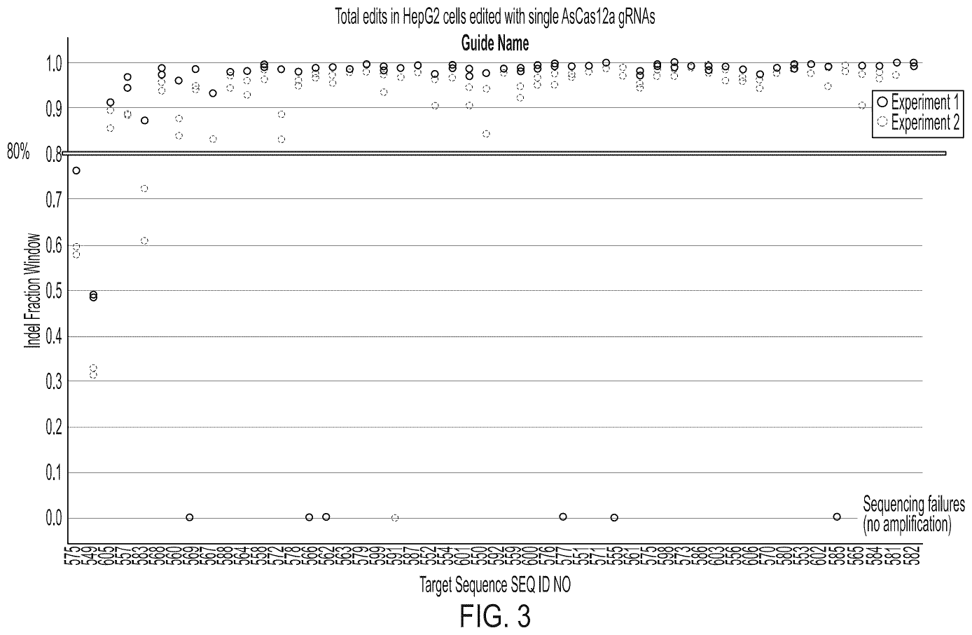

In certain embodiments, a gRNA complexed to an unmodified or modified Cas12a protein may be modified to increase the editing efficiency of a target nucleic acid. In certain embodiments, the modified gRNA may comprise one or more modifications including a phosphorothioate (PS2) linkage modification, a 2′-O-methyl modification (non-limiting exemplary modifications are illustrated in FIG. 3), one or more or a stretch of additional nucleotides (e.g., RNA or deoxyribonucleic acid (DNA) nucleotides) not found in a corresponding native gRNA (also referred herein as a “gRNA extension”), or combinations thereof.

In some embodiments, a gRNA used herein, e.g., a Cas12a gRNA, comprises one or more or a stretch of additional ribonucleic acid or deoxyribonucleic acid (DNA) bases outside of the spacer region, also referred to herein as a “gRNA extension.” In some embodiments, a gRNA used herein comprises a gRNA extension that comprises one or more or a stretch of DNA bases, referred to herein as a “DNA extension”. In some embodiments, a gRNA used herein comprises a DNA extension at the 5′ end of the gRNA, the 3′ end of the gRNA, or a combination thereof. In certain embodiments, the DNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 DNA bases long. For example, in certain embodiments, the DNA extension may be 1, 2, 3, 4, 5, 10, 15, 20, or 25 DNA bases long. In certain embodiments, the DNA extension may comprise one or more DNA bases selected from adenine (A), guanine (G), cytosine (C), or thymine (T). In certain embodiments, the DNA extension comprises the same DNA bases. For example, the DNA extension may comprise a stretch of adenine (A) bases. In certain embodiments, the DNA extension may comprise a stretch of thymine (T) bases. In certain embodiments, the DNA extension comprises a combination of different DNA bases. In certain embodiments, a DNA extension may comprise or consist of a sequence set forth in Table 3. In certain embodiments, a gRNA used herein comprises a DNA extension as well as one or more phosphorothioate linkage modifications, one or more phosphorodithioate (PS2) linkage modifications, one or more 2′-O-methyl modifications, or combinations thereof. In certain embodiments, the one or more modifications may be at the 5′ end of the gRNA, at the 3′ end of the gRNA, or combinations thereof. In certain embodiments, a gRNA including a DNA extension may comprise a sequence set forth in Table 3 that comprises a DNA extension. Without wishing to be bound by theory, it is contemplated that any DNA extension may be used herein, so long as it does not hybridize to the target nucleic acid being targeted by the gRNA. In some embodiments the DNA extension additionally exhibits an increase in editing efficiency, e.g., via changes to gRNA stability, uptake, and/or activity, at the target nucleic acid site relative to a gRNA which does not comprise such a DNA extension.

In some embodiments, a gRNA used herein, e.g., a Cas12a gRNA, comprises a gRNA extension that comprises one or more or a stretch of ribonucleic acid (RNA) bases, also referred to herein as an “RNA extension.” In some embodiments, a gRNA used herein comprises an RNA extension at the 5′ end of the gRNA, the 3′ end of the gRNA, or a combination thereof. In certain embodiments, the RNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 RNA bases long. For example, in certain embodiments, the RNA extension may be 1, 2, 3, 4, 5, 10, 15, 20, or 25 RNA bases long. In certain embodiments, the RNA extension may comprise one or more RNA bases selected from adenine (rA), guanine (rG), cytosine (rC), or uracil (rU), in which the “r” represents RNA, 2′-hydroxy. In certain embodiments, the RNA extension comprises the same RNA bases. For example, the RNA extension may comprise a stretch of adenine (rA) bases. In certain embodiments, the RNA extension comprises a combination of different RNA bases. In certain embodiments, an RNA extension may comprise or consist of a sequence set forth in Table 3. In certain embodiments, a gRNA used herein comprises an RNA extension as well as one or more phosphorothioate linkage modifications, one or more phosphorodithioate (PS2) linkage modifications, one or more 2′-O-methyl modifications, or combinations thereof. In certain embodiments, the one or more modifications may be at the 5′ end of the gRNA, at the 3′ end of the gRNA, or combinations thereof. In certain embodiments, a gRNA including an RNA extension may comprise a sequence set forth in Table 3 that comprises an RNA extension. gRNAs including an RNA extension at the 5′ end of the gRNA may comprise a sequence disclosed herein. gRNAs including an RNA extension at the 3′ end of the gRNA may comprise a sequence disclosed herein.

It is contemplated that gRNAs used herein may also comprise an RNA extension and a DNA extension. In certain embodiments, the RNA extension and DNA extension may both be at the 5′ end of the gRNA, the 3′ end of the gRNA, or a combination thereof. In certain embodiments, the RNA extension is at the 5′ end of the gRNA and the DNA extension is at the 3′ end of the gRNA. In certain embodiments, the RNA extension is at the 3′ end of the gRNA and the DNA extension is at the 5′ end of the gRNA.

It is further contemplated that gRNAs used herein may comprise a gRNA extension that is a hybrid extension that comprises both deoxyribonucleic acid and ribonucleic acid moieties.

In some embodiments a gRNA which comprises a modification, e.g., a DNA extension at the 5′ end, is complexed with a RNA-guided nuclease, e.g., an AsCas12a nuclease, to form an RNP complex, (such RNP complex formation occurring either prior to delivery of a composition described herein to a subject or following such delivery, e.g., in a cell after expression of an mRNA encoding the RNA-guided nuclease), which then edits a target cell (e.g., a liver cell). Exemplary suitable 5′ extensions for guide RNAs, e.g., Cas12a guide RNAs are provided in the table below:

TABLE 3
gRNA 5′ Extensions

| SEQ | | |
|---|---|---|
| ID | | 5′ |
| NO | 5′ extension sequence | modification |
| 1 | rCrUrUrUrU | +5 RNA |
| 2 | rArArGrArCrCrUrUrUrU | +10 RNA |
| 3 | rArUrGrUrGrUrUrUrUrUrGrUrC | +25 RNA |
| | rArArArArGrArCrCrUrUrUrU | |
| 4 | rArGrGrCrCrArGrCrUrUrGrCrC | +60 RNA |
| | rGrGrUrUrUrUrUrUrArGrUrCrG | |
| | rUrGrCrUrGrCrUrUrCrArUrGrU | |
| | rGrUrUrUrUrUrGrUrCrArArArA | |
| | rGrArCrCrUrUrUrU | |
| 5 | CTTTT | +5 DNA |
| 6 | AAGACCTTTT | +10 DNA |
| 7 | ATGTGTTTTTGTCAAAAGACCTTTT | +25 DNA |
| 8 | AGGCCAGCTTGCCGGTTTTTTAGTCG | +60 DNA |
| | TGCTGCTTCATGTG | |
| 9 | TTTTTGTCAAAAGACCTTTT | +20 DNA |
| 10 | GCTTCATGTGTTTTTGTCAAAAGACC | +30 DNA |
| | TTTT | |
| 11 | GCCGGTTTTTTAGTCGTGCTGCTTCA | +50 DNA |
| | TGTGTTTTTGTCAAAAGACCTTTT | |
| 12 | TAGTCGTGCTGCTTCATGTGTTTTTG | +40 DNA |
| | TCAAAAGACCTTTT | |
| 13 | C*C*GAAGTTTTCTTCGGTTTT | +20 DNA + 2xPS |
| 14 | T*T*TTTCCGAAGTTTTCTTCGGTTT | +25 DNA + 2xPS |
| | T | |
| 15 | A*A*CGCTTTTTCCGAAGTTTTCTTC | +30 DNA + 2xPS |
| | GGTTTT | |
| 16 | G*C*GTTGTTTTCAACGCTTTTTCCG | +41 DNA + 2xPS |
| | AAGTTTTCTTCGGTTTT | |
| 17 | G*G*CTTCTTTTGAAGCCTTTTTGCG | +62 DNA + 2xPS |
| | TTGTTTTCAACGCTTTTTCCGAAGTT | |
| | TTCTTCGGTTTT | |
| 18 | A*T*GTGTTTTTGTCAAAAGACCTTT | +25 DNA + 2xPS |
| | T | |
| 19 | AAAAAAAAAAAAAAAAAAAAAAAAA | +25 A |
| 20 | TTTTTTTTTTTTTTTTTTTTTTTTT | +25 T |
| 21 | mA*mU*rGrUrGrUrUrUrUrUrGrU | +25 RNA + 2xPS |
| | rCrArArArArGrArCrCrUrUrUrU | |
| 22 | mA*mA*rArArArArArArArArArA | PolyA RNA + |
| | rArArArArArArArArArArArArA | 2xPS |
| 23 | mU*mU*rUrUrUrUrUrUrUrUrUrU | PolyU RNA + |
| | rUrUrUrUrUrUrUrUrUrUrUrUrU | 2xPS |

All bases are in upper case Lowercase “r” represents RNA, 2′-hydroxy; bases not modified by an “r” are DNA
All bases are linked via standard phosphodiester bonds except as noted:
“*” represents phosphorothioate modification
“PS” represents phosphorothioate modification

Additional suitable gRNA modifications will be apparent to those of ordinary skill in the art based on the present disclosure. Suitable gRNA modifications comprise, for example, those described in PCT application PCT/US2018/054027, filed on Oct. 2, 2018, and entitled “MODIFIED CPF1 GUIDE RNA;” in PCT application PCT/US2015/000143, filed on Dec. 3, 2015, and entitled “GUIDE RNA WITH CHEMICAL MODIFICATIONS;” in PCT application PCT/US2016/026028, filed Apr. 5, 2016, and entitled “CHEMICALLY MODIFIED GUIDE RNAS FOR CRISPR/CAS-MEDIATED GENE REGULATION;” and in PCT application PCT/US2016/053344, filed on Sep. 23, 2016, and entitled “NUCLEASE-MEDIATED GENOME EDITING OF PRIMARY CELLS AND ENRICHMENT THEREOF;” the entire contents of each of which are incorporated herein by reference. Without being bound by theory, in certain embodiments of genome editing systems of the present disclosure, the one or more modifications of the gRNA enhance binding affinity of the gRNA molecule to RNA-guided nuclease of the genome editing system, e.g., a Cas12a nuclease.

### 4. Guide RNA Design

Methods for selection and validation of target sequences as well as off-target analyses have been described previously, e.g., in Mali; Hsu; Fu et al., 2014 Nat Biotechnol 32 (3): 279-84, Heigwer et al., 2014 Nat methods 11 (2): 122-3; Bae et al. (2014) Bioinformatics 30 (10): 1473-5; Xiao A et al. (2014) Bioinformatics 30 (8): 1180-1182; and Fennel et al (2021) CRISPR J 4 (2): 264-274. Each of these references is incorporated by reference herein. In certain non-limiting embodiments, gRNA design can involve the use of a software tool to optimize the choice of potential target sequences corresponding to a user's target sequence, e.g., to minimize total off-target activity across the genome. These and other guide selection methods are described in detail in Maeder and Cotta-Ramusino.

In certain embodiments, one or more or all of the nucleotides in a gRNA are modified. Strategies for modifying a gRNA are described in WO2019/152519, published Aug. 8, 2019, the entire contents of which are expressly incorporated herein by reference.

Non-limiting examples of guide RNAs suitable for certain embodiments embraced by the present disclosure are provided herein, for example, in the Tables below. Those of ordinary skill in the art will be able to envision suitable guide RNA sequences for a specific nuclease, e.g., a Cas9 or Cas12a nuclease, from the disclosure of the targeting domain sequence, either as a DNA or RNA sequence. For example, a guide RNA comprising a targeting domain consisting of RNA nucleotides would comprise the RNA sequence corresponding to the targeting domain sequence provided as a DNA sequence, and thus contain uracil instead of thymidine nucleotides. For example, a guide RNA comprising a targeting domain sequence consisting of RNA nucleotides and described by the DNA sequence TTCCTTTCCTCGTGAAGGATA (SEQ ID NO: 552) would have a targeting domain of the corresponding RNA sequence rUrUrCrCrUrUrUrCrCrUrCrGrUrGrArArGrGrArUrA (SEQ ID NO: 610). As will be apparent to the skilled artisan, such a targeting domain would be linked to a suitable guide RNA scaffold, e.g., a crRNA scaffold sequence or a chimeric crRNA/tracrRNA scaffold sequence. Suitable gRNA scaffold sequences are known to those of ordinary skill in the art. For AsCas12a, for example, a suitable scaffold sequence comprises the sequence rUrArArUrUrUrCrUrArCrUrCrUrUrGrUrArGrArU (SEQ ID NO: 27), added to the 5′-terminus of the targeting domain. In the example above, this would result in a Cas12a guide RNA of the sequence; rUrUrCrCrUrUrUrCrCrUrCrGrUrGrArArGrGrArUrArUrArArUrUrUrCrUrArCrUrCrUrUrG rUrArGrArU (SEQ ID NO: 28). Those of skill in the art would further understand how to modify such a guide RNA. For example, adding a 25-mer DNA extension (e.g., SEQ ID NO: 7) would result, for example, a in guide RNA of the sequence ATGTGTTTTTGTCAAAAGACCTTTTrUrUrCrCrUrUrUrCrCrUrCrGrUrGrArArGrGrArU rArUrArArUrUrUrCrUrArCrUrCrUrUrGrUrArGrArU (SEQ ID NO: 29). It will be understood that the exemplary targeting domain sequences provided herein are not limiting, and additional suitable sequences, e.g., variants of the specific sequences disclosed herein, will be apparent to the skilled artisan based on the present disclosure in view of the general knowledge in the art.

In some embodiments, the gRNA for use in the disclosure is a gRNA targeting LDLR (LDLR gRNA). In some embodiments, the LDLR cDNA sequence is SEQ ID NO: 1483:

GTGCAATCGCGGGAAGCCAGGGTTTCCAGCTAGGACACAGCAGGTCGTGATCCG
GGTCGGGACACTGCCTGGCAGAGGCTGCGAGCATGGGGCCCTGGGGCTGGAAAT
TGCGCTGGACCGTCGCCTTGCTCCTCGCCGCGGCGGGGACTGCAGTGGGCGACA
GATGCGAAAGAAACGAGTTCCAGTGCCAAGACGGGAAATGCATCTCCTACAAGT
GGGTCTGCGATGGCAGCGCTGAGTGCCAGGATGGCTCTGATGAGTCCCAGGAGA
CGTGCTTGTCTGTCACCTGCAAATCCGGGGACTTCAGCTGTGGGGGCCGTGTCAA
CCGCTGCATTCCTCAGTTCTGGAGGTGCGATGGCCAAGTGGACTGCGACAACGG
CTCAGACGAGCAAGGCTGTCCCCCCAAGACGTGCTCCCAGGACGAGTTTCGCTG
CCACGATGGGAAGTGCATCTCTCGGCAGTTCGTCTGTGACTCAGACCGGGACTGC
TTGGACGGCTCAGACGAGGCCTCCTGCCCGGTGCTCACCTGTGGTCCCGCCAGCT
TCCAGTGCAACAGCTCCACCTGCATCCCCCAGCTGTGGGCCTGCGACAACGACCC
CGACTGCGAAGATGGCTCGGATGAGTGGCCGCAGCGCTGTAGGGGTCTTTACGT
GTTCCAAGGGGACAGTAGCCCCTGCTCGGCCTTCGAGTTCCACTGCCTAAGTGGC
GAGTGCATCCACTCCAGCTGGCGCTGTGATGGTGGCCCCGACTGCAAGGACAAA
TCTGACGAGGAAAACTGCGCTGTGGCCACCTGTCGCCCTGACGAATTCCAGTGCT
CTGATGGAAACTGCATCCATGGCAGCCGGCAGTGTGACCGGGAATATGACTGCA
AGGACATGAGCGATGAAGTTGGCTGCGTTAATGTGACACTCTGCGAGGGACCCA
ACAAGTTCAAGTGTCACAGCGGCGAATGCATCACCCTGGACAAAGTCTGCAACA
TGGCTAGAGACTGCCGGGACTGGTCAGATGAACCCATCAAAGAGTGCGGGACCA
ACGAATGCTTGGACAACAACGGCGGCTGTTCCCACGTCTGCAATGACCTTAAGAT
CGGCTACGAGTGCCTGTGCCCCGACGGCTTCCAGCTGGTGGCCCAGCGAAGATG
CGAAGATATCGATGAGTGTCAGGATCCCGACACCTGCAGCCAGCTCTGCGTGAA
CCTGGAGGGTGGCTACAAGTGCCAGTGTGAGGAAGGCTTCCAGCTGGACCCCCA
CACGAAGGCCTGCAAGGCTGTGGGCTCCATCGCCTACCTCTTCTTCACCAACCGG
CACGAGGTCAGGAAGATGACGCTGGACCGGAGCGAGTACACCAGCCTCATCCCC
AACCTGAGGAACGTGGTCGCTCTGGACACGGAGGTGGCCAGCAATAGAATCTAC
TGGTCTGACCTGTCCCAGAGAATGATCTGCAGCACCCAGCTTGACAGAGCCCAC
GGCGTCTCTTCCTATGACACCGTCATCAGCAGAGACATCCAGGCCCCCGACGGGC
TGGCTGTGGACTGGATCCACAGCAACATCTACTGGACCGACTCTGTCCTGGGCAC
TGTCTCTGTTGCGGATACCAAGGGCGTGAAGAGGAAAACGTTATTCAGGGAGAA
CGGCTCCAAGCCAAGGGCCATCGTGGTGGATCCTGTTCATGGCTTCATGTACTGG
ACTGACTGGGGAACTCCCGCCAAGATCAAGAAAGGGGGCCTGAATGGTGTGGAC
ATCTACTCGCTGGTGACTGAAAACATTCAGTGGCCCAATGGCATCACCCTAGATC
TCCTCAGTGGCCGCCTCTACTGGGTTGACTCCAAACTTCACTCCATCTCAAGCAT
CGATGTCAACGGGGGCAACCGGAAGACCATCTTGGAGGATGAAAAGAGGCTGG
CCCACCCCTTCTCCTTGGCCGTCTTTGAGGACAAAGTATTTTGGACAGATATCAT
CAACGAAGCCATTTTCAGTGCCAACCGCCTCACAGGTTCCGATGTCAACTTGTTG
GCTGAAAACCTACTGTCCCCAGAGGATATGGTTCTCTTCCACAACCTCACCCAGC
CAAGAGGAGTGAACTGGTGTGAGAGGACCACCCTGAGCAATGGCGGCTGCCAGT
ATCTGTGCCTCCCTGCCCCGCAGATCAACCCCCACTCGCCCAAGTTTACCTGCGC
CTGCCCGGACGGCATGCTGCTGGCCAGGGACATGAGGAGCTGCCTCACAGAGGC
TGAGGCTGCAGTGGCCACCCAGGAGACATCCACCGTCAGGCTAAAGGTCAGCTC
CACAGCCGTAAGGACACAGCACACAACCACCCGACCTGTTCCCGACACCTCCCG
GCTGCCTGGGGCCACCCCTGGGCTCACCACGGTGGAGATAGTGACAATGTCTCA
CCAAGCTCTGGGCGACGTTGCTGGCAGAGGAAATGAGAAGAAGCCCAGTAGCGT
GAGGGCTCTGTCCATTGTCCTCCCCATCGTGCTCCTCGTCTTCCTTTGCCTGGGGG
TCTTCCTTCTATGGAAGAACTGGCGGCTTAAGAACATCAACAGCATCAACTTTGA
CAACCCCGTCTATCAGAAGACCACAGAGGATGAGGTCCACATTTGCCACAACCA
GGACGGCTACAGCTACCCCTCGAGACAGATGGTCAGTCTGGAGGATGACGTGGC
GTGAACATCTGCCTGGAGTCCCGTCCCTGCCCAGAACCCTTCCTGAGACCTCGCC
GGCCTTGTTTTATTCAAAGACAGAGAAGACCAAAGCATTGCCTGCCAGAGCTTTG
TTTTATATATTTATTCATCTGGGAGGCAGAACAGGCTTCGGACAGTGCCCATGCA
ATGGCTTGGGTTGGGATTTTGGTTTCTTCCTTTCCTCGTGAAGGATAAGAGAAAC
AGGCCCGGGGGGACCAGGATGACACCTCCATTTCTCTCCAGGAAGTTTTGAGTTT
CTCTCCACCGTGACACAATCCTCAAACATGGAAGATGAAAGGGGAGGGGATGTC
AGGCCCAGAGAAGCAAGTGGCTTTCAACACACAACAGCAGATGGCACCAACGG
GACCCCCTGGCCCTGCCTCATCCACCAATCTCTAAGCCAAACCCCTAAACTCAGG
AGTCAACGTGTTTACCTCTTCTATGCAAGCCTTGCTAGACAGCCAGGTTAGCCTT
TGCCCTGTCACCCCCGAATCATGACCCACCCAGTGTCTTTCGAGGTGGGTTTGTA
CCTTCCTTAAGCCAGGAAAGGGATTCATGGCGTCGGAAATGATCTGGCTGAATCC
GTGGTGGCACCGAGACCAAACTCATTCACCAAATGATGCCACTTCCCAGAGGCA
GAGCCTGAGTCACTGGTCACCCTTAATATTTATTAAGTGCCTGAGACACCCGGTT
ACCTTGGCCGTGAGGACACGTGGCCTGCACCCAGGTGTGGCTGTCAGGACACCA
GCCTGGTGCCCATCCTCCCGACCCCTACCCACTTCCATTCCCGTGGTCTCCTTGCA
CTTTCTCAGTTCAGAGTTGTACACTGTGTACATTTGGCATTTGTGTTATTATTTTG
CACTGTTTTCTGTCGTGTGTGTTGGGATGGGATCCCAGGCCAGGGAAAGCCCGTG
TCAATGAATGCCGGGGACAGAGAGGGGCAGGTTGACCGGGACTTCAAAGCCGTG
ATCGTGAATATCGAGAACTGCCATTGTCGTCTTTATGTCCGCCCACCTAGTGCTTC
CACTTCTATGCAAATGCCTCCAAGCCATTCACTTCCCCAATCTTGTCGTTGATGGG
TATGTGTTTAAAACATGCACGGTGAGGCCGGGCGCAGTGGCTCACGCCTGTAATC
CCAGCACTTTGGGAGGCCGAGGCGGGTGGATCATGAGGTCAGGAGATCGAGACC
ATCCTGGCTAACACGTGAAACCCCGTCTCTACTAAAAATACAAAAAATTAGCCG
GGCGTGGTGGCGGGCACCTGTAGTCCCAGCTACTCGGGAGGCTGAGGCAGGAGA
ATGGTGTGAACCCGGGAAGCGGAGCTTGCAGTGAGCCGAGATTGCGCCACTGCA
GTCCGCAGTCTGGCCTGGGCGACAGAGCGAGACTCCGTCTCAAAAAAAAAAAAC
AAAAAAAAACCATGCATGGTGCATCAGCAGCCCATGGCCTCTGGCCAGGCATGG
CGAGGCTGAGGTGGGAGGATGGTTTGAGCTCAGGCATTTGAGGCTGTCGTGAGC
TATGATTATGCCACTGCTTTCCAGCCTGGGCAACATAGTAAGACCCCATCTCTTA
AAAAATGAATTTGGCCAGACACAGGTGCCTCACGCCTGTAATCCCAGCACTTTGG
GAGGCTGAGCTGGATCACTTGAGTTCAGGAGTTGGAGACCAGGCCTGAGCAACA
AAGCGAGATCCCATCTCTACAAAAACCAAAAAGTTAAAAATCAGCTGGGTACGG
TGGCACGTGCCTGTGATCCCAGCTACTTGGGAGGCTGAGGCAGGAGGATCGCCT
GAGCCCAGGAGGTGGAGGTTGCAGTGAGCCATGATCGAGCCACTGCACTCCAGC
CTGGGCAACAGATGAAGACCCTATTTCAGAAATACAACTATAAAAAAATAAATA
AATCCTCCAGTCTGGATCGTTTGACGGGACTTCAGGTTCTTTCTGAAATCGCCGT
GTTACTGTTGCACTGATGTCCGGAGAGACAGTGACAGCCTCCGTCAGACTCCCGC
GTGAAGATGTCACAAGGGATTGGCAATTGTCCCCAGGGACAAAACACTGTGTCC
CCCCCAGTGCAGGGAACCGTGATAAGCCTTTCTGGTTTCGGAGCACGTAAATGCG
TCCCTGTACAGATAGTGGGGATTTTTTGTTATGTTTGCACTTTGTATATTGGTTGA
AACTGTTATCACTTATATATATATATATACACACATATATATAAAATCTATTTATT
TTTGCAAACCCTGGTTGCTGTATTTGTTCAGTGACTATTCTCGGGGCCCTGTGTAG
GGGGTTATTGCCTCTGAAATGCCTCTTCTTTATGTACAAAGATTATTTGCACGAAC
TGGACTGTGTGCAACGCTTTTTGGGAGAATGATGTCCCCGTTGTATGTATGAGTG
GCTTCTGGGAGATGGGTGTCACTTTTTAAACCACTGTATAGAAGGTTTTTGTAGC
CTGAATGTCTTACTGTGATCAATTAAATTTCTTAAATGAACCAA

In some embodiments, the target sequence of an LDLR gene comprises or consists of a nucleotide sequence set forth in SEQ ID NOs: 61-304; 549-606; 665-750; or 837-865 (Tables 4, 7, 10, 13). In certain embodiments, the target sequence of an LDLR gene comprises or consists of the nucleotide sequence set forth in SEQ ID NOs: 61, 72, 73, 74, 288, 300, 552, 553, 576, 585, 589, or 602. In certain embodiments, the target sequence of an LDLR gene comprises or consists of the nucleotide sequence set forth in SEQ ID NOs: 552 or 585. In some embodiments, the targeting domain of the gRNA molecule comprises or consists of a nucleotide sequence set forth in SEQ ID NOs: 305-548; 607-664; 751-836; or 866-894 (Tables 5, 8, 11, 14). In certain embodiments, the targeting domain of the gRNA targeting an LDLR gene is SEQ ID NO: 610, or SEQ ID NO: 643. In some embodiments, the gRNA molecule targeting an LDLR gene comprises or consists of a nucleotide sequence set forth in SEQ ID NOs: 900-1316, or 1331-1337 (Tables 6, 9, 12, and 15). In certain embodiments, the gRNA molecule targeting an LDLR gene comprises or consists of the sequence set forth in SEQ ID NOs: 900-913, 1114-1138, or 1139-1141. An exemplary LDLR gene target sequence, gRNA targeting domain, scaffold sequence, and DNA extension are set forth in Table 16A-16B.

TABLE 4
LDLR Target Sequences for SpCas9

| SEQ ID NO | Target Sequence (DNA) |
|---|---|
| 61 | ACGTGGCGTGAACATCTGCC |
| 62 | CTGGGCAGGGACGGGACTCC |
| 63 | GAGGTCTCAGGAAGGGTTCT |
| 64 | CGAGGTCTCAGGAAGGGTTC |
| 65 | AACCCTTCCTGAGACCTCGC |
| 66 | GGCCGGCGAGGTCTCAGGAA |
| 67 | AGGCCGGCGAGGTCTCAGGA |
| 68 | AACAAGGCCGGCGAGGTCTC |
| 69 | TGAATAAAACAAGGCCGGCG |
| 70 | GTCTTTGAATAAAACAAGGC |
| 71 | CTCTGTCTTTGAATAAAACA |
| 72 | AGCTCTGGCAGGCAATGCTT |
| 73 | ATATAAAACAAAGCTCTGGC |
| 74 | AAATATATAAAACAAAGCTC |
| 75 | ATTCATCTGGGAGGCAGAAC |
| 76 | CTGGGAGGCAGAACAGGCTT |
| 77 | TTCGGACAGTGCCCATGCAA |
| 78 | ACAGTGCCCATGCAATGGCT |
| 79 | CAGTGCCCATGCAATGGCTT |
| 80 | GCCCATGCAATGGCTTGGGT |
| 81 | CCCATGCAATGGCTTGGGTT |
| 82 | CCCAACCCAAGCCATTGCAT |
| 83 | TCCCAACCCAAGCCATTGCA |
| 84 | GTTTCTTCCTTTCCTCGTGA |
| 85 | CTCTTATCCTTCACGAGGAA |
| 86 | TGTTTCTCTTATCCTTCACG |
| 87 | AGGATAAGAGAAACAGGCCC |
| 88 | GGATAAGAGAAACAGGCCCG |
| 89 | GATAAGAGAAACAGGCCCGG |
| 90 | ATAAGAGAAACAGGCCCGGG |
| 91 | GAAACAGGCCCGGGGGGACC |
| 92 | GGTGTCATCCTGGTCCCCCC |
| 93 | AGGTGTCATCCTGGTCCCCC |
| 94 | GAGAAATGGAGGTGTCATCC |
| 95 | TGACACCTCCATTTCTCTCC |
| 96 | AACTTCCTGGAGAGAAATGG |
| 97 | CAAAACTTCCTGGAGAGAAA |
| 98 | GAGAGAAACTCAAAACTTCC |
| 99 | GTTTGAGGATTGTGTCACGG |
| 100 | CGTGACACAATCCTCAAACA |
| 101 | CATGTTTGAGGATTGTGTCA |
| 102 | CCTCAAACATGGAAGATGAA |
| 103 | CTCAAACATGGAAGATGAAA |
| 104 | TCAAACATGGAAGATGAAAG |
| 105 | CCTTTCATCTTCCATGTTTG |
| 106 | GTCAGGCCCAGAGAAGCAAG |
| 107 | TGAAAGCCACTTGCTTCTCT |
| 108 | TTGAAAGCCACTTGCTTCTC |
| 109 | TTTCAACACACAACAGCAGA |
| 110 | ACAACAGCAGATGGCACCAA |
| 111 | CAACAGCAGATGGCACCAAC |
| 112 | ATGGCACCAACGGGACCCCC |
| 113 | GCAGGGCCAGGGGGTCCCGT |
| 114 | GTGGATGAGGCAGGGCCAGG |
| 115 | AGAGATTGGTGGATGAGGCA |
| 116 | GGCTTAGAGATTGGTGGATG |
| 117 | GGGTTTGGCTTAGAGATTGG |
| 118 | TAGGGGTTTGGCTTAGAGAT |
| 119 | TAAGCCAAACCCCTAAACTC |
| 120 | GACTCCTGAGTTTAGGGGTT |
| 121 | ACGTTGACTCCTGAGTTTAG |
| 122 | CACGTTGACTCCTGAGTTTA |
| 123 | ACACGTTGACTCCTGAGTTT |
| 124 | GCAAGCCTTGCTAGACAGCC |
| 125 | GCTAACCTGGCTGTCTAGCA |
| 126 | TGACAGGGCAAAGGCTAACC |
| 127 | ATTCGGGGGTGACAGGGCAA |
| 128 | GTCATGATTCGGGGGTGACA |
| 129 | GGTCATGATTCGGGGGTGAC |
| 130 | CTGGGTGGGTCATGATTCGG |
| 131 | ACTGGGTGGGTCATGATTCG |
| 132 | CACTGGGTGGGTCATGATTC |
| 133 | ACACTGGGTGGGTCATGATT |
| 134 | GACCCACCCAGTGTCTTTCG |
| 135 | CCACCCAGTGTCTTTCGAGG |
| 136 | CACCCAGTGTCTTTCGAGGT |
| 137 | CACCTCGAAAGACACTGGGT |
| 138 | AACCCACCTCGAAAGACACT |
| 139 | AAACCCACCTCGAAAGACAC |
| 140 | GTTTGTACCTTCCTTAAGCC |
| 141 | TACCTTCCTTAAGCCAGGAA |
| 142 | ACCTTCCTTAAGCCAGGAAA |
| 143 | TCCCTTTCCTGGCTTAAGGA |
| 144 | TGAATCCCTTTCCTGGCTTA |
| 145 | TAAGCCAGGAAAGGGATTCA |
| 146 | AGGAAAGGGATTCATGGCGT |
| 147 | GACGCCATGAATCCCTTTCC |
| 148 | TCATGGCGTCGGAAATGATC |
| 149 | AAATGATCTGGCTGAATCCG |
| 150 | TGATCTGGCTGAATCCGTGG |
| 151 | GTTTGGTCTCGGTGCCACCA |
| 152 | TTGGTGAATGAGTTTGGTCT |
| 153 | CATCATTTGGTGAATGAGTT |
| 154 | CAAATGATGCCACTTCCCAG |
| 155 | CTCTGGGAAGTGGCATCATT |
| 156 | CAGGCTCTGCCTCTGGGAAG |
| 157 | AGAGGCAGAGCCTGAGTCAC |
| 158 | AGTGACTCAGGCTCTGCCTC |
| 159 | TTAAGGGTGACCAGTGACTC |
| 160 | CAGGCACTTAATAAATATTA |
| 161 | TATTAAGTGCCTGAGACACC |
| 162 | CCTGAGACACCCGGTTACCT |
| 163 | CCAAGGTAACCGGGTGTCTC |
| 164 | ACCCGGTTACCTTGGCCGTG |
| 165 | TCCTCACGGCCAAGGTAACC |
| 166 | GTCCTCACGGCCAAGGTAAC |
| 167 | ACCTTGGCCGTGAGGACACG |
| 168 | GCCACGTGTCCTCACGGCCA |
| 169 | GTGCAGGCCACGTGTCCTCA |
| 170 | AGGACACGTGGCCTGCACCC |
| 171 | ACGTGGCCTGCACCCAGGTG |
| 172 | TGCACCCAGGTGTGGCTGTC |
| 173 | TGACAGCCACACCTGGGTGC |
| 174 | GTGTCCTGACAGCCACACCT |
| 175 | GGTGTCCTGACAGCCACACC |
| 176 | TGGCTGTCAGGACACCAGCC |
| 177 | CGGGAGGATGGGCACCAGGC |
| 178 | GGGTCGGGAGGATGGGCACC |
| 179 | TGGGTAGGGGTCGGGAGGAT |
| 180 | GGAAGTGGGTAGGGGTCGGG |
| 181 | CCTACCCACTTCCATTCCCG |
| 182 | ACGGGAATGGAAGTGGGTAG |
| 183 | CACGGGAATGGAAGTGGGTA |
| 184 | CCACGGGAATGGAAGTGGGT |
| 185 | GAGACCACGGGAATGGAAGT |
| 186 | GGAGACCACGGGAATGGAAG |
| 187 | GTGCAAGGAGACCACGGGAA |
| 188 | AGAAAGTGCAAGGAGACCAC |
| 189 | GAGAAAGTGCAAGGAGACCA |
| 190 | CTCTGAACTGAGAAAGTGCA |
| 191 | AGTTGTACACTGTGTACATT |
| 192 | TGTTTTCTGTCGTGTGTGTT |
| 193 | TTCTGTCGTGTGTGTTGGGA |
| 194 | TCTGTCGTGTGTGTTGGGAT |
| 195 | GTGTGTTGGGATGGGATCCC |
| 196 | ACACGGGCTTTCCCTGGCCT |
| 197 | GACACGGGCTTTCCCTGGCC |
| 198 | TCATTGACACGGGCTTTCCC |
| 199 | AAGCCCGTGTCAATGAATGC |
| 200 | AGCCCGTGTCAATGAATGCC |
| 201 | GCCCGTGTCAATGAATGCCG |
| 202 | TCCCCGGCATTCATTGACAC |
| 203 | GTCCCCGGCATTCATTGACA |
| 204 | AATGAATGCCGGGGACAGAG |
| 205 | ATGAATGCCGGGGACAGAGA |
| 206 | TGCCGGGGACAGAGAGGGGC |
| 207 | AACCTGCCCCTCTCTGTCCC |
| 208 | ACAGAGAGGGGCAGGTTGAC |
| 209 | CAGAGAGGGGCAGGTTGACC |
| 210 | CGATCACGGCTTTGAAGTCC |
| 211 | GTTCTCGATATTCACGATCA |
| 212 | GGCGGACATAAAGACGACAA |
| 213 | AAGTGGAAGCACTAGGTGGG |
| 214 | TAGAAGTGGAAGCACTAGGT |
| 215 | ATAGAAGTGGAAGCACTAGG |
| 216 | TGCATAGAAGTGGAAGCACT |
| 217 | TGGAGGCATTTGCATAGAAG |
| 218 | TGGGGAAGTGAATGGCTTGG |
| 219 | GATTGGGGAAGTGAATGGCT |
| 220 | GACAAGATTGGGGAAGTGAA |
| 221 | TTCCCCAATCTTGTCGTTGA |
| 222 | TCCCCAATCTTGTCGTTGAT |
| 223 | ACCCATCAACGACAAGATTG |
| 224 | TACCCATCAACGACAAGATT |
| 225 | GTATGTGTTTAAAACATGCA |
| 226 | TGTTTAAAACATGCACGGTG |
| 227 | TAAAACATGCACGGTGAGGC |
| 228 | AAAACATGCACGGTGAGGCC |
| 229 | GGCTGCTGATGCACCATGCA |
| 230 | CATGGTGCATCAGCAGCCCA |
| 231 | CATCAGCAGCCCATGGCCTC |
| 232 | CATGCCTGGCCAGAGGCCAT |
| 233 | GCCTCTGGCCAGGCATGGCG |
| 234 | GCCTCGCCATGCCTGGCCAG |
| 235 | CATCTCTTAAAAAATGAATT |
| 236 | AAATGAATTTGGCCAGACAC |
| 237 | TATAGTTGTATTTCTGAAAT |
| 238 | CTCCAGTCTGGATCGTTTGA |
| 239 | TCCAGTCTGGATCGTTTGAC |
| 240 | CGTCAAACGATCCAGACTGG |
| 241 | TCCCGTCAAACGATCCAGAC |
| 242 | GGATCGTTTGACGGGACTTC |
| 243 | ACATCAGTGCAACAGTAACA |
| 244 | GGAGGCTGTCACTGTCTCTC |
| 245 | TTCACGCGGGAGTCTGACGG |
| 246 | ATCTTCACGCGGGAGTCTGA |
| 247 | TCCCGCGTGAAGATGTCACA |
| 248 | CCCGCGTGAAGATGTCACAA |
| 249 | CCCTTGTGACATCTTCACGC |
| 250 | TCCCTTGTGACATCTTCACG |
| 251 | GTGAAGATGTCACAAGGGAT |
| 252 | AGGGATTGGCAATTGTCCCC |
| 253 | GGGATTGGCAATTGTCCCCA |
| 254 | GACACAGTGTTTTGTCCCTG |
| 255 | GGACACAGTGTTTTGTCCCT |
| 256 | GGGACACAGTGTTTTGTCCC |
| 257 | CACTGTGTCCCCCCCAGTGC |
| 258 | ACTGTGTCCCCCCCAGTGCA |
| 259 | CACGGTTCCCTGCACTGGGG |
| 260 | TCACGGTTCCCTGCACTGGG |
| 261 | ATCACGGTTCCCTGCACTGG |
| 262 | TATCACGGTTCCCTGCACTG |
| 263 | TTATCACGGTTCCCTGCACT |
| 264 | CTTATCACGGTTCCCTGCAC |
| 265 | GGAACCGTGATAAGCCTTTC |
| 266 | GTGATAAGCCTTTCTGGTTT |
| 267 | GAAACCAGAAAGGCTTATCA |
| 268 | TACGTGCTCCGAAACCAGAA |
| 269 | ATGCGTCCCTGTACAGATAG |
| 270 | TGCGTCCCTGTACAGATAGT |
| 271 | GCGTCCCTGTACAGATAGTG |
| 272 | AAATCCCCACTATCTGTACA |
| 273 | AAAATCCCCACTATCTGTAC |
| 274 | TATGTTTGCACTTTGTATAT |
| 275 | CTATTTATTTTTGCAAACCC |
| 276 | CTGAACAAATACAGCAACCA |
| 277 | ACTGAACAAATACAGCAACC |
| 278 | ATTTGTTCAGTGACTATTCT |
| 279 | TTTGTTCAGTGACTATTCTC |
| 280 | TTGTTCAGTGACTATTCTCG |
| 281 | CTATTCTCGGGGCCCTGTGT |
| 282 | TATTCTCGGGGCCCTGTGTA |
| 283 | ATTCTCGGGGCCCTGTGTAG |
| 284 | TTCTCGGGGCCCTGTGTAGG |
| 285 | AGGCAATAACCCCCTACACA |
| 286 | GAGGCAATAACCCCCTACAC |
| 287 | ATAAAGAAGAGGCATTTCAG |
| 288 | AATCTTTGTACATAAAGAAG |
| 289 | CAAAGATTATTTGCACGAAC |
| 290 | GGACTGTGTGCAACGCTTTT |
| 291 | GACTGTGTGCAACGCTTTTT |
| 292 | GTCCCCGTTGTATGTATGAG |
| 293 | AGCCACTCATACATACAACG |
| 294 | AAGCCACTCATACATACAAC |
| 295 | GAAGCCACTCATACATACAA |
| 296 | TTGTATGTATGAGTGGCTTC |
| 297 | TGTATGTATGAGTGGCTTCT |
| 298 | TATGAGTGGCTTCTGGGAGA |
| 299 | ATGAGTGGCTTCTGGGAGAT |
| 300 | TACAAAAACCTTCTATACAG |
| 301 | TTGATCACAGTAAGACATTC |
| 302 | TGCATCGAGTTTAGACAAAT |
| 303 | TTAGCGTTGGCCACCCGCGG |
| 304 | AAATTAGCGTTGGCCACCCG |
| 1317 | TTGAAATTCGAATTCTGCCT |
| 1318 | GTGCGTTGCTTTGAGTGGGT |
| 1365 | ACAGATGACAGAAGGAAGTG |
| 1366 | TTCCTTCTGTCATCTGTTTG |
| 1367 | TCTGTCATCTGTTTGTGGAT |
| 1368 | ATCCACAAACAGATGACAGA |
| 1369 | GTTTGTGGATTGGACTCCCC |
| 1370 | GGATTGGACTCCCCAGGAGA |
| 1371 | GGGAAGACTCCCAGAACTCC |
| 1372 | GTGTATGAATGGATGAGCAG |
| 1373 | AGTGTATGAATGGATGAGCA |
| 1374 | ATATTTGTCGAGTGTATGAA |
| 1375 | CACAAGCCTACGGCAGAGCA |
| 1376 | GTAGGCTTGTGGGACACTAC |
| 1377 | TAGGCTTGTGGGACACTACA |
| 1378 | TGTAGTGTCCCACAAGCCTA |
| 1379 | AGGCTTGTGGGACACTACAG |
| 1380 | AAAGAAATGAAGTCTTGATA |
| 1381 | GAAATGAAGTCTTGATACGG |
| 1382 | AATCTCCCTGTCTGTCCTTA |
| 1383 | TCAGGTCCTTAAGGACAGAC |
| 1384 | AAAATGATGTCAGGTCCTTA |
| 1385 | CCTCAAGTCGTCTACCGCCT |
| 1386 | CCGAGGCGGTAGACGACTTG |
| 1387 | TTCTCTAATGAAGTAACATT |
| 1388 | CAGAAAAGCAACTGGCAGGA |
| 1389 | GGGACAGAAAAGCAACTGGC |
| 1390 | TATGGGGACAGAAAAGCAAC |
| 1391 | TTTTGTATCTATTCTAAAAA |
| 1392 | TGTATCTATTCTAAAAATGG |
| 1461 | GAGCAACACAGAGCCCAGGG |

TABLE 5
LDLR Targeting Sequences for SpCas9

| SEQ | |
|---|---|
| ID NO | gDNA targeting Domain (RNA) |
| 305 | rArCrGrUrGrGrCrGrUrGrArArCrArUrCrUrGrCrC |
| 306 | rCrUrGrGrGrCrArGrGrGrArCrGrGrGrArCrUrCrC |
| 307 | rGrArGrGrUrCrUrCrArGrGrArArGrGrGrUrUrCrU |
| 308 | rCrGrArGrGrUrCrUrCrArGrGrArArGrGrGrUrUrC |
| 309 | rArArCrCrCrUrUrCrCrUrGrArGrArCrCrUrCrGrC |
| 310 | rGrGrCrCrGrGrCrGrArGrGrUrCrUrCrArGrGrArA |
| 311 | rArGrGrCrCrGrGrCrGrArGrGrUrCrUrCrArGrGrA |
| 312 | rArArCrArArGrGrCrCrGrGrCrGrArGrGrUrCrUrC |
| 313 | rUrGrArArUrArArArArCrArArGrGrCrCrGrGrCrG |
| 314 | rGrUrCrUrUrUrGrArArUrArArArArCrArArGrGrC |
| 315 | rCrUrCrUrGrUrCrUrUrUrGrArArUrArArArArCrA |
| 316 | rArGrCrUrCrUrGrGrCrArGrGrCrArArUrGrCrUrU |
| 317 | rArUrArUrArArArArCrArArArGrCrUrCrUrGrGrC |
| 318 | rArArArUrArUrArUrArArArArCrArArArGrCrUrC |
| 319 | rArUrUrCrArUrCrUrGrGrGrArGrGrCrArGrArArC |
| 320 | rCrUrGrGrGrArGrGrCrArGrArArCrArGrGrCrUrU |
| 321 | rUrUrCrGrGrArCrArGrUrGrCrCrCrArUrGrCrArA |
| 322 | rArCrArGrUrGrCrCrCrArUrGrCrArArUrGrGrCrU |
| 323 | rCrArGrUrGrCrCrCrArUrGrCrArArUrGrGrCrUrU |
| 324 | rGrCrCrCrArUrGrCrArArUrGrGrCrUrUrGrGrGrU |
| 325 | rCrCrCrArUrGrCrArArUrGrGrCrUrUrGrGrGrUrU |
| 326 | rCrCrCrArArCrCrCrArArGrCrCrArUrUrGrCrArU |
| 327 | rUrCrCrCrArArCrCrCrArArGrCrCrArUrUrGrCrA |
| 328 | rGrUrUrUrCrUrUrCrCrUrUrUrCrCrUrCrGrUrGrA |
| 329 | rCrUrCrUrUrArUrCrCrUrUrCrArCrGrArGrGrArA |
| 330 | rUrGrUrUrUrCrUrCrUrUrArUrCrCrUrUrCrArCrG |
| 331 | rArGrGrArUrArArGrArGrArArArCrArGrGrCrCrC |
| 332 | rGrGrArUrArArGrArGrArArArCrArGrGrCrCrCrG |
| 333 | rGrArUrArArGrArGrArArArCrArGrGrCrCrCrGrG |
| 334 | rArUrArArGrArGrArArArCrArGrGrCrCrCrGrGrG |
| 335 | rGrArArArCrArGrGrCrCrCrGrGrGrGrGrGrArCrC |
| 336 | rGrGrUrGrUrCrArUrCrCrUrGrGrUrCrCrCrCrCrC |
| 337 | rArGrGrUrGrUrCrArUrCrCrUrGrGrUrCrCrCrCrC |
| 338 | rGrArGrArArArUrGrGrArGrGrUrGrUrCrArUrCrC |
| 339 | rUrGrArCrArCrCrUrCrCrArUrUrUrCrUrCrUrCrC |
| 340 | rArArCrUrUrCrCrUrGrGrArGrArGrArArArUrGrG |
| 341 | rCrArArArArCrUrUrCrCrUrGrGrArGrArGrArArA |
| 342 | rGrArGrArGrArArArCrUrCrArArArArCrUrUrCrC |
| 343 | rGrUrUrUrGrArGrGrArUrUrGrUrGrUrCrArCrGrG |
| 344 | rCrGrUrGrArCrArCrArArUrCrCrUrCrArArArCrA |
| 345 | rCrArUrGrUrUrUrGrArGrGrArUrUrGrUrGrUrCrA |
| 346 | rCrCrUrCrArArArCrArUrGrGrArArGrArUrGrArA |
| 347 | rCrUrCrArArArCrArUrGrGrArArGrArUrGrArArA |
| 348 | rUrCrArArArCrArUrGrGrArArGrArUrGrArArArG |
| 349 | rCrCrUrUrUrCrArUrCrUrUrCrCrArUrGrUrUrUrG |
| 350 | rGrUrCrArGrGrCrCrCrArGrArGrArArGrCrArArG |
| 351 | rUrGrArArArGrCrCrArCrUrUrGrCrUrUrCrUrCrU |
| 352 | rUrUrGrArArArGrCrCrArCrUrUrGrCrUrUrCrUrC |
| 353 | rUrUrUrCrArArCrArCrArCrArArCrArGrCrArGrA |
| 354 | rArCrArArCrArGrCrArGrArUrGrGrCrArCrCrArA |
| 355 | rCrArArCrArGrCrArGrArUrGrGrCrArCrCrArArC |
| 356 | rArUrGrGrCrArCrCrArArCrGrGrGrArCrCrCrCrC |
| 357 | rGrCrArGrGrGrCrCrArGrGrGrGrGrUrCrCrCrGrU |
| 358 | rGrUrGrGrArUrGrArGrGrCrArGrGrGrCrCrArGrG |
| 359 | rArGrArGrArUrUrGrGrUrGrGrArUrGrArGrGrCrA |
| 360 | rGrGrCrUrUrArGrArGrArUrUrGrGrUrGrGrArUrG |
| 361 | rGrGrGrUrUrUrGrGrCrUrUrArGrArGrArUrUrGrG |
| 362 | rUrArGrGrGrGrUrUrUrGrGrCrUrUrArGrArGrArU |
| 363 | rUrArArGrCrCrArArArCrCrCrCrUrArArArCrUrC |
| 364 | rGrArCrUrCrCrUrGrArGrUrUrUrArGrGrGrGrUrU |
| 365 | rArCrGrUrUrGrArCrUrCrCrUrGrArGrUrUrUrArG |
| 366 | rCrArCrGrUrUrGrArCrUrCrCrUrGrArGrUrUrUrA |
| 367 | rArCrArCrGrUrUrGrArCrUrCrCrUrGrArGrUrUrU |
| 368 | rGrCrArArGrCrCrUrUrGrCrUrArGrArCrArGrCrC |
| 369 | rGrCrUrArArCrCrUrGrGrCrUrGrUrCrUrArGrCrA |
| 370 | rUrGrArCrArGrGrGrCrArArArGrGrCrUrArArCrC |
| 371 | rArUrUrCrGrGrGrGrGrUrGrArCrArGrGrGrCrArA |
| 372 | rGrUrCrArUrGrArUrUrCrGrGrGrGrGrUrGrArCrA |
| 373 | rGrGrUrCrArUrGrArUrUrCrGrGrGrGrGrUrGrArC |
| 374 | rCrUrGrGrGrUrGrGrGrUrCrArUrGrArUrUrCrGrG |
| 375 | rArCrUrGrGrGrUrGrGrGrUrCrArUrGrArUrUrCrG |
| 376 | rCrArCrUrGrGrGrUrGrGrGrUrCrArUrGrArUrUrC |
| 377 | rArCrArCrUrGrGrGrUrGrGrGrUrCrArUrGrArUrU |
| 378 | rGrArCrCrCrArCrCrCrArGrUrGrUrCrUrUrUrCrG |
| 379 | rCrCrArCrCrCrArGrUrGrUrCrUrUrUrCrGrArGrG |
| 380 | rCrArCrCrCrArGrUrGrUrCrUrUrUrCrGrArGrGrU |
| 381 | rCrArCrCrUrCrGrArArArGrArCrArCrUrGrGrGrU |
| 382 | rArArCrCrCrArCrCrUrCrGrArArArGrArCrArCrU |
| 383 | rArArArCrCrCrArCrCrUrCrGrArArArGrArCrArC |
| 384 | rGrUrUrUrGrUrArCrCrUrUrCrCrUrUrArArGrCrC |
| 385 | rUrArCrCrUrUrCrCrUrUrArArGrCrCrArGrGrArA |
| 386 | rArCrCrUrUrCrCrUrUrArArGrCrCrArGrGrArArA |
| 387 | rUrCrCrCrUrUrUrCrCrUrGrGrCrUrUrArArGrGrA |
| 388 | rUrGrArArUrCrCrCrUrUrUrCrCrUrGrGrCrUrUrA |
| 389 | rUrArArGrCrCrArGrGrArArArGrGrGrArUrUrCrA |
| 390 | rArGrGrArArArGrGrGrArUrUrCrArUrGrGrCrGrU |
| 391 | rGrArCrGrCrCrArUrGrArArUrCrCrCrUrUrUrCrC |
| 392 | rUrCrArUrGrGrCrGrUrCrGrGrArArArUrGrArUrC |
| 393 | rArArArUrGrArUrCrUrGrGrCrUrGrArArUrCrCrG |
| 394 | rUrGrArUrCrUrGrGrCrUrGrArArUrCrCrGrUrGrG |
| 395 | rGrUrUrUrGrGrUrCrUrCrGrGrUrGrCrCrArCrCrA |
| 396 | rUrUrGrGrUrGrArArUrGrArGrUrUrUrGrGrUrCrU |
| 397 | rCrArUrCrArUrUrUrGrGrUrGrArArUrGrArGrUrU |
| 398 | rCrArArArUrGrArUrGrCrCrArCrUrUrCrCrCrArG |
| 399 | rCrUrCrUrGrGrGrArArGrUrGrGrCrArUrCrArUrU |
| 400 | rCrArGrGrCrUrCrUrGrCrCrUrCrUrGrGrGrArArG |
| 401 | rArGrArGrGrCrArGrArGrCrCrUrGrArGrUrCrArC |
| 402 | rArGrUrGrArCrUrCrArGrGrCrUrCrUrGrCrCrUrC |
| 403 | rUrUrArArGrGrGrUrGrArCrCrArGrUrGrArCrUrC |
| 404 | rCrArGrGrCrArCrUrUrArArUrArArArUrArUrUrA |
| 405 | rUrArUrUrArArGrUrGrCrCrUrGrArGrArCrArCrC |
| 406 | rCrCrUrGrArGrArCrArCrCrCrGrGrUrUrArCrCrU |
| 407 | rCrCrArArGrGrUrArArCrCrGrGrGrUrGrUrCrUrC |
| 408 | rArCrCrCrGrGrUrUrArCrCrUrUrGrGrCrCrGrUrG |
| 409 | rUrCrCrUrCrArCrGrGrCrCrArArGrGrUrArArCrC |
| 410 | rGrUrCrCrUrCrArCrGrGrCrCrArArGrGrUrArArC |
| 411 | rArCrCrUrUrGrGrCrCrGrUrGrArGrGrArCrArCrG |
| 412 | rGrCrCrArCrGrUrGrUrCrCrUrCrArCrGrGrCrCrA |
| 413 | rGrUrGrCrArGrGrCrCrArCrGrUrGrUrCrCrUrCrA |
| 414 | rArGrGrArCrArCrGrUrGrGrCrCrUrGrCrArCrCrC |
| 415 | rArCrGrUrGrGrCrCrUrGrCrArCrCrCrArGrGrUrG |
| 416 | rUrGrCrArCrCrCrArGrGrUrGrUrGrGrCrUrGrUrC |
| 417 | rUrGrArCrArGrCrCrArCrArCrCrUrGrGrGrUrGrC |
| 418 | rGrUrGrUrCrCrUrGrArCrArGrCrCrArCrArCrCrU |
| 419 | rGrGrUrGrUrCrCrUrGrArCrArGrCrCrArCrArCrC |
| 420 | rUrGrGrCrUrGrUrCrArGrGrArCrArCrCrArGrCrC |
| 421 | rCrGrGrGrArGrGrArUrGrGrGrCrArCrCrArGrGrC |
| 422 | rGrGrGrUrCrGrGrGrArGrGrArUrGrGrGrCrArCrC |
| 423 | rUrGrGrGrUrArGrGrGrGrUrCrGrGrGrArGrGrArU |
| 424 | rGrGrArArGrUrGrGrGrUrArGrGrGrGrUrCrGrGrG |
| 425 | rCrCrUrArCrCrCrArCrUrUrCrCrArUrUrCrCrCrG |
| 426 | rArCrGrGrGrArArUrGrGrArArGrUrGrGrGrUrArG |
| 427 | rCrArCrGrGrGrArArUrGrGrArArGrUrGrGrGrUrA |
| 428 | rCrCrArCrGrGrGrArArUrGrGrArArGrUrGrGrGrU |
| 429 | rGrArGrArCrCrArCrGrGrGrArArUrGrGrArArGrU |
| 430 | rGrGrArGrArCrCrArCrGrGrGrArArUrGrGrArArG |
| 431 | rGrUrGrCrArArGrGrArGrArCrCrArCrGrGrGrArA |
| 432 | rArGrArArArGrUrGrCrArArGrGrArGrArCrCrArC |
| 433 | rGrArGrArArArGrUrGrCrArArGrGrArGrArCrCrA |
| 434 | rCrUrCrUrGrArArCrUrGrArGrArArArGrUrGrCrA |
| 435 | rArGrUrUrGrUrArCrArCrUrGrUrGrUrArCrArUrU |
| 436 | rUrGrUrUrUrUrCrUrGrUrCrGrUrGrUrGrUrGrUrU |
| 437 | rUrUrCrUrGrUrCrGrUrGrUrGrUrGrUrUrGrGrGrA |
| 438 | rUrCrUrGrUrCrGrUrGrUrGrUrGrUrUrGrGrGrArU |
| 439 | rGrUrGrUrGrUrUrGrGrGrArUrGrGrGrArUrCrCrC |
| 440 | rArCrArCrGrGrGrCrUrUrUrCrCrCrUrGrGrCrCrU |
| 441 | rGrArCrArCrGrGrGrCrUrUrUrCrCrCrUrGrGrCrC |
| 442 | rUrCrArUrUrGrArCrArCrGrGrGrCrUrUrUrCrCrC |
| 443 | rArArGrCrCrCrGrUrGrUrCrArArUrGrArArUrGrC |
| 444 | rArGrCrCrCrGrUrGrUrCrArArUrGrArArUrGrCrC |
| 445 | rGrCrCrCrGrUrGrUrCrArArUrGrArArUrGrCrCrG |
| 446 | rUrCrCrCrCrGrGrCrArUrUrCrArUrUrGrArCrArC |
| 447 | rGrUrCrCrCrCrGrGrCrArUrUrCrArUrUrGrArCrA |
| 448 | rArArUrGrArArUrGrCrCrGrGrGrGrArCrArGrArG |
| 449 | rArUrGrArArUrGrCrCrGrGrGrGrArCrArGrArGrA |
| 450 | rUrGrCrCrGrGrGrGrArCrArGrArGrArGrGrGrGrC |
| 451 | rArArCrCrUrGrCrCrCrCrUrCrUrCrUrGrUrCrCrC |
| 452 | rArCrArGrArGrArGrGrGrGrCrArGrGrUrUrGrArC |
| 453 | rCrArGrArGrArGrGrGrGrCrArGrGrUrUrGrArCrC |
| 454 | rCrGrArUrCrArCrGrGrCrUrUrUrGrArArGrUrCrC |
| 455 | rGrUrUrCrUrCrGrArUrArUrUrCrArCrGrArUrCrA |
| 456 | rGrGrCrGrGrArCrArUrArArArGrArCrGrArCrArA |
| 457 | rArArGrUrGrGrArArGrCrArCrUrArGrGrUrGrGrG |
| 458 | rUrArGrArArGrUrGrGrArArGrCrArCrUrArGrGrU |
| 459 | rArUrArGrArArGrUrGrGrArArGrCrArCrUrArGrG |
| 460 | rUrGrCrArUrArGrArArGrUrGrGrArArGrCrArCrU |
| 461 | rUrGrGrArGrGrCrArUrUrUrGrCrArUrArGrArArG |
| 462 | rUrGrGrGrGrArArGrUrGrArArUrGrGrCrUrUrGrG |
| 463 | rGrArUrUrGrGrGrGrArArGrUrGrArArUrGrGrCrU |
| 464 | rGrArCrArArGrArUrUrGrGrGrGrArArGrUrGrArA |
| 465 | rUrUrCrCrCrCrArArUrCrUrUrGrUrCrGrUrUrGrA |
| 466 | rUrCrCrCrCrArArUrCrUrUrGrUrCrGrUrUrGrArU |
| 467 | rArCrCrCrArUrCrArArCrGrArCrArArGrArUrUrG |
| 468 | rUrArCrCrCrArUrCrArArCrGrArCrArArGrArUrU |
| 469 | rGrUrArUrGrUrGrUrUrUrArArArArCrArUrGrCrA |
| 470 | rUrGrUrUrUrArArArArCrArUrGrCrArCrGrGrUrG |
| 471 | rUrArArArArCrArUrGrCrArCrGrGrUrGrArGrGrC |
| 472 | rArArArArCrArUrGrCrArCrGrGrUrGrArGrGrCrC |
| 473 | rGrGrCrUrGrCrUrGrArUrGrCrArCrCrArUrGrCrA |
| 474 | rCrArUrGrGrUrGrCrArUrCrArGrCrArGrCrCrCrA |
| 475 | rCrArUrCrArGrCrArGrCrCrCrArUrGrGrCrCrUrC |
| 476 | rCrArUrGrCrCrUrGrGrCrCrArGrArGrGrCrCrArU |
| 477 | rGrCrCrUrCrUrGrGrCrCrArGrGrCrArUrGrGrCrG |
| 478 | rGrCrCrUrCrGrCrCrArUrGrCrCrUrGrGrCrCrArG |
| 479 | rCrArUrCrUrCrUrUrArArArArArArUrGrArArUrU |
| 480 | rArArArUrGrArArUrUrUrGrGrCrCrArGrArCrArC |
| 481 | rUrArUrArGrUrUrGrUrArUrUrUrCrUrGrArArArU |
| 482 | rCrUrCrCrArGrUrCrUrGrGrArUrCrGrUrUrUrGrA |
| 483 | rUrCrCrArGrUrCrUrGrGrArUrCrGrUrUrUrGrArC |
| 484 | rCrGrUrCrArArArCrGrArUrCrCrArGrArCrUrGrG |
| 485 | rUrCrCrCrGrUrCrArArArCrGrArUrCrCrArGrArC |
| 486 | rGrGrArUrCrGrUrUrUrGrArCrGrGrGrArCrUrUrC |
| 487 | rArCrArUrCrArGrUrGrCrArArCrArGrUrArArCrA |
| 488 | rGrGrArGrGrCrUrGrUrCrArCrUrGrUrCrUrCrUrC |
| 489 | rUrUrCrArCrGrCrGrGrGrArGrUrCrUrGrArCrGrG |
| 490 | rArUrCrUrUrCrArCrGrCrGrGrGrArGrUrCrUrGrA |
| 491 | rUrCrCrCrGrCrGrUrGrArArGrArUrGrUrCrArCrA |
| 492 | rCrCrCrGrCrGrUrGrArArGrArUrGrUrCrArCrArA |
| 493 | rCrCrCrUrUrGrUrGrArCrArUrCrUrUrCrArCrGrC |
| 494 | rUrCrCrCrUrUrGrUrGrArCrArUrCrUrUrCrArCrG |
| 495 | rGrUrGrArArGrArUrGrUrCrArCrArArGrGrGrArU |
| 496 | rArGrGrGrArUrUrGrGrCrArArUrUrGrUrCrCrCrC |
| 497 | rGrGrGrArUrUrGrGrCrArArUrUrGrUrCrCrCrCrA |
| 498 | rGrArCrArCrArGrUrGrUrUrUrUrGrUrCrCrCrUrG |
| 499 | rGrGrArCrArCrArGrUrGrUrUrUrUrGrUrCrCrCrU |
| 500 | rGrGrGrArCrArCrArGrUrGrUrUrUrUrGrUrCrCrC |
| 501 | rCrArCrUrGrUrGrUrCrCrCrCrCrCrCrArGrUrGrC |
| 502 | rArCrUrGrUrGrUrCrCrCrCrCrCrCrArGrUrGrCrA |
| 503 | rCrArCrGrGrUrUrCrCrCrUrGrCrArCrUrGrGrGrG |
| 504 | rUrCrArCrGrGrUrUrCrCrCrUrGrCrArCrUrGrGrG |
| 505 | rArUrCrArCrGrGrUrUrCrCrCrUrGrCrArCrUrGrG |
| 506 | rUrArUrCrArCrGrGrUrUrCrCrCrUrGrCrArCrUrG |
| 507 | rUrUrArUrCrArCrGrGrUrUrCrCrCrUrGrCrArCrU |
| 508 | rCrUrUrArUrCrArCrGrGrUrUrCrCrCrUrGrCrArC |
| 509 | rGrGrArArCrCrGrUrGrArUrArArGrCrCrUrUrUrC |
| 510 | rGrUrGrArUrArArGrCrCrUrUrUrCrUrGrGrUrUrU |
| 511 | rGrArArArCrCrArGrArArArGrGrCrUrUrArUrCrA |
| 512 | rUrArCrGrUrGrCrUrCrCrGrArArArCrCrArGrArA |
| 513 | rArUrGrCrGrUrCrCrCrUrGrUrArCrArGrArUrArG |
| 514 | rUrGrCrGrUrCrCrCrUrGrUrArCrArGrArUrArGrU |
| 515 | rGrCrGrUrCrCrCrUrGrUrArCrArGrArUrArGrUrG |
| 516 | rArArArUrCrCrCrCrArCrUrArUrCrUrGrUrArCrA |
| 517 | rArArArArUrCrCrCrCrArCrUrArUrCrUrGrUrArC |
| 518 | rUrArUrGrUrUrUrGrCrArCrUrUrUrGrUrArUrArU |
| 519 | rCrUrArUrUrUrArUrUrUrUrUrGrCrArArArCrCrC |
| 520 | rCrUrGrArArCrArArArUrArCrArGrCrArArCrCrA |
| 521 | rArCrUrGrArArCrArArArUrArCrArGrCrArArCrC |
| 522 | rArUrUrUrGrUrUrCrArGrUrGrArCrUrArUrUrCrU |
| 523 | rUrUrUrGrUrUrCrArGrUrGrArCrUrArUrUrCrUrC |
| 524 | rUrUrGrUrUrCrArGrUrGrArCrUrArUrUrCrUrCrG |
| 525 | rCrUrArUrUrCrUrCrGrGrGrGrCrCrCrUrGrUrGrU |
| 526 | rUrArUrUrCrUrCrGrGrGrGrCrCrCrUrGrUrGrUrA |
| 527 | rArUrUrCrUrCrGrGrGrGrCrCrCrUrGrUrGrUrArG |
| 528 | rUrUrCrUrCrGrGrGrGrCrCrCrUrGrUrGrUrArGrG |
| 529 | rArGrGrCrArArUrArArCrCrCrCrCrUrArCrArCrA |
| 530 | rGrArGrGrCrArArUrArArCrCrCrCrCrUrArCrArC |
| 531 | rArUrArArArGrArArGrArGrGrCrArUrUrUrCrArG |
| 532 | rArArUrCrUrUrUrGrUrArCrArUrArArArGrArArG |
| 533 | rCrArArArGrArUrUrArUrUrUrGrCrArCrGrArArC |
| 534 | rGrGrArCrUrGrUrGrUrGrCrArArCrGrCrUrUrUrU |
| 535 | rGrArCrUrGrUrGrUrGrCrArArCrGrCrUrUrUrUrU |
| 536 | rGrUrCrCrCrCrGrUrUrGrUrArUrGrUrArUrGrArG |
| 537 | rArGrCrCrArCrUrCrArUrArCrArUrArCrArArCrG |
| 538 | rArArGrCrCrArCrUrCrArUrArCrArUrArCrArArC |
| 539 | rGrArArGrCrCrArCrUrCrArUrArCrArUrArCrArA |
| 540 | rUrUrGrUrArUrGrUrArUrGrArGrUrGrGrCrUrUrC |
| 541 | rUrGrUrArUrGrUrArUrGrArGrUrGrGrCrUrUrCrU |
| 542 | rUrArUrGrArGrUrGrGrCrUrUrCrUrGrGrGrArGrA |
| 543 | rArUrGrArGrUrGrGrCrUrUrCrUrGrGrGrArGrArU |
| 544 | rUrArCrArArArArArCrCrUrUrCrUrArUrArCrArG |
| 545 | rUrUrGrArUrCrArCrArGrUrArArGrArCrArUrUrC |
| 546 | rUrGrCrArUrCrGrArGrUrUrUrArGrArCrArArArU |
| 547 | rUrUrArGrCrGrUrUrGrGrCrCrArCrCrCrGrCrGrG |
| 548 | rArArArUrUrArGrCrGrUrUrGrGrCrCrArCrCrCrG |
| 1324 | rUrUrGrArArArUrUrCrGrArArUrUrCrUrGrCrCrU |
| 1325 | rGrUrGrCrGrUrUrGrCrUrUrUrGrArGrUrGrGrGrU |
| 1393 | rArCrArGrArUrGrArCrArGrArArGrGrArArGrUrG |
| 1394 | rUrUrCrCrUrUrCrUrGrUrCrArUrCrUrGrUrUrUrG |
| 1395 | rUrCrUrGrUrCrArUrCrUrGrUrUrUrGrUrGrGrArU |
| 1396 | rArUrCrCrArCrArArArCrArGrArUrGrArCrArGrA |
| 1397 | rGrUrUrUrGrUrGrGrArUrUrGrGrArCrUrCrCrCrC |
| 1398 | rGrGrArUrUrGrGrArCrUrCrCrCrCrArGrGrArGrA |
| 1399 | rGrGrGrArArGrArCrUrCrCrCrArGrArArCrUrCrC |
| 1400 | rGrUrGrUrArUrGrArArUrGrGrArUrGrArGrCrArG |
| 1401 | rArGrUrGrUrArUrGrArArUrGrGrArUrGrArGrCrA |
| 1402 | rArUrArUrUrUrGrUrCrGrArGrUrGrUrArUrGrArA |
| 1403 | rCrArCrArArGrCrCrUrArCrGrGrCrArGrArGrCrA |
| 1404 | rGrUrArGrGrCrUrUrGrUrGrGrGrArCrArCrUrArC |
| 1405 | rUrArGrGrCrUrUrGrUrGrGrGrArCrArCrUrArCrA |
| 1406 | rUrGrUrArGrUrGrUrCrCrCrArCrArArGrCrCrUrA |
| 1407 | rArGrGrCrUrUrGrUrGrGrGrArCrArCrUrArCrArG |
| 1408 | rArArArGrArArArUrGrArArGrUrCrUrUrGrArUrA |
| 1409 | rGrArArArUrGrArArGrUrCrUrUrGrArUrArCrGrG |
| 1410 | rArArUrCrUrCrCrCrUrGrUrCrUrGrUrCrCrUrUrA |
| 1411 | rUrCrArGrGrUrCrCrUrUrArArGrGrArCrArGrArC |
| 1412 | rArArArArUrGrArUrGrUrCrArGrGrUrCrCrUrUrA |
| 1413 | rCrCrUrCrArArGrUrCrGrUrCrUrArCrCrGrCrCrU |
| 1414 | rCrCrGrArGrGrCrGrGrUrArGrArCrGrArCrUrUrG |
| 1415 | rUrUrCrUrCrUrArArUrGrArArGrUrArArCrArUrU |
| 1416 | rCrArGrArArArArGrCrArArCrUrGrGrCrArGrGrA |
| 1417 | rGrGrGrArCrArGrArArArArGrCrArArCrUrGrGrC |
| 1418 | rUrArUrGrGrGrGrArCrArGrArArArArGrCrArArC |
| 1419 | rUrUrUrUrGrUrArUrCrUrArUrUrCrUrArArArArA |
| 1420 | rUrGrUrArUrCrUrArUrUrCrUrArArArArArUrGrG |
| 1455 | mC*mA*mG*rUrGrCrCrCrArUrGrCrArArUrGrGrCrUrU |
| 1456 | mA*mU*mG*rArGrUrGrGrCrUrUrCrUrGrGrGrArGrArU |
| 1462 | mG*mA*mG*rCrArArCrArCrArGrArGrCrCrCrArGrGrG |
| 1464 | mU*mU*mG*rArArArUrUrCrGrArArUrUrCrUrGrCrCrU |
| 1466 | mG*mU*mG*rCrGrUrUrGrCrUrUrUrGrArGrUrGrGrGrU |
| 1468 | mC*mA*mG*rTrGrCrCrCrArTrGrCrArArTrGrGrCrTrT |
| 1470 | mA*mT*mG*rArGrTrGrGrCrTrTrCrTrGrGrGrArGrArT |
| 1451 | mA*mG*mC*rUrCrUrGrGrCrArGrGrCrArArUrGrCrUrU |
| 1452 | mA*mU*mA*rUrArArArArCrArArArGrCrUrCrUrGrGrC |
| 1453 | mA*mA*mA*rUrArUrArUrArArArArCrArArArGrCrUrC |
| 1454 | mA*mC*mG*rUrGrGrCrGrUrGrArArCrArUrCrUrGrCrC |
| 1474 | mA*mA*mU*rCrUrUrUrGrUrArCrArUrArArArGrArArG |
| 1475 | mU*mA*mC*rArArArArArCrCrUrUrCrUrArUrArCrArG |

All bases are in upper case Lowercase “r” represents RNA, 2′-hydroxy; bases not modified by an “r” are DNA
All bases are linked via standard phosphodiester bonds except as noted:
“*” represents phosphorothioate modification

TABLE 6
gRNA sequences for targeting LDLR with SpCas9

| SEQ | |
|---|---|
| ID NO | gRNA sequence |
| 900 | rArCrGrUrGrGrCrGrUrGrArArCrArUrCrUrGrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 901 | rCrUrGrGrGrCrArGrGrGrArCrGrGrGrArCrUrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 902 | rGrArGrGrUrCrUrCrArGrGrArArGrGrGrUrUrCrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 903 | rCrGrArGrGrUrCrUrCrArGrGrArArGrGrGrUrUrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 904 | rArArCrCrCrUrUrCrCrUrGrArGrArCrCrUrCrGrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 905 | rGrGrCrCrGrGrCrGrArGrGrUrCrUrCrArGrGrArArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 906 | rArGrGrCrCrGrGrCrGrArGrGrUrCrUrCrArGrGrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 907 | rArArCrArArGrGrCrCrGrGrCrGrArGrGrUrCrUrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 908 | rUrGrArArUrArArArArCrArArGrGrCrCrGrGrCrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 909 | rGrUrCrUrUrUrGrArArUrArArArArCrArArGrGrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 910 | rCrUrCrUrGrUrCrUrUrUrGrArArUrArArArArCrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 911 | rArGrCrUrCrUrGrGrCrArGrGrCrArArUrGrCrUrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 912 | rArUrArUrArArArArCrArArArGrCrUrCrUrGrGrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 913 | rArArArUrArUrArUrArArArArCrArArArGrCrUrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 914 | rArUrUrCrArUrCrUrGrGrGrArGrGrCrArGrArArCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 915 | rCrUrGrGrGrArGrGrCrArGrArArCrArGrGrCrUrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 916 | rUrUrCrGrGrArCrArGrUrGrCrCrCrArUrGrCrArArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 917 | rArCrArGrUrGrCrCrCrArUrGrCrArArUrGrGrCrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 918 | rCrArGrUrGrCrCrCrArUrGrCrArArUrGrGrCrUrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 919 | rGrCrCrCrArUrGrCrArArUrGrGrCrUrUrGrGrGrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 920 | rCrCrCrArUrGrCrArArUrGrGrCrUrUrGrGrGrUrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 921 | rCrCrCrArArCrCrCrArArGrCrCrArUrUrGrCrArUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 922 | rUrCrCrCrArArCrCrCrArArGrCrCrArUrUrGrCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 923 | rGrUrUrUrCrUrUrCrCrUrUrUrCrCrUrCrGrUrGrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 924 | rCrUrCrUrUrArUrCrCrUrUrCrArCrGrArGrGrArArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 925 | rUrGrUrUrUrCrUrCrUrUrArUrCrCrUrUrCrArCrGrGrUrUrUrUrArGrArGrCrU |
| | ArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 926 | rArGrGrArUrArArGrArGrArArArCrArGrGrCrCrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 927 | rGrGrArUrArArGrArGrArArArCrArGrGrCrCrCrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 928 | rGrArUrArArGrArGrArArArCrArGrGrCrCrCrGrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 929 | rArUrArArGrArGrArArArCrArGrGrCrCrCrGrGrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 930 | rGrArArArCrArGrGrCrCrCrGrGrGrGrGrGrArCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 931 | rGrGrUrGrUrCrArUrCrCrUrGrGrUrCrCrCrCrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 932 | rArGrGrUrGrUrCrArUrCrCrUrGrGrUrCrCrCrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 933 | rGrArGrArArArUrGrGrArGrGrUrGrUrCrArUrCrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 934 | rUrGrArCrArCrCrUrCrCrArUrUrUrCrUrCrUrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 935 | rArArCrUrUrCrCrUrGrGrArGrArGrArArArUrGrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 936 | rCrArArArArCrUrUrCrCrUrGrGrArGrArGrArArArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 937 | rGrArGrArGrArArArCrUrCrArArArArCrUrUrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 938 | rGrUrUrUrGrArGrGrArUrUrGrUrGrUrCrArCrGrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 939 | rCrGrUrGrArCrArCrArArUrCrCrUrCrArArArCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 940 | rCrArUrGrUrUrUrGrArGrGrArUrUrGrUrGrUrCrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 941 | rCrCrUrCrArArArCrArUrGrGrArArGrArUrGrArArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 942 | rCrUrCrArArArCrArUrGrGrArArGrArUrGrArArArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 943 | rUrCrArArArCrArUrGrGrArArGrArUrGrArArArGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 944 | rCrCrUrUrUrCrArUrCrUrUrCrCrArUrGrUrUrUrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 945 | rGrUrCrArGrGrCrCrCrArGrArGrArArGrCrArArGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 946 | rUrGrArArArGrCrCrArCrUrUrGrCrUrUrCrUrCrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 947 | rUrUrGrArArArGrCrCrArCrUrUrGrCrUrUrCrUrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 948 | rUrUrUrCrArArCrArCrArCrArArCrArGrCrArGrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 949 | rArCrArArCrArGrCrArGrArUrGrGrCrArCrCrArArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 950 | rCrArArCrArGrCrArGrArUrGrGrCrArCrCrArArCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 951 | rArUrGrGrCrArCrCrArArCrGrGrGrArCrCrCrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 952 | rGrCrArGrGrGrCrCrArGrGrGrGrGrUrCrCrCrGrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 953 | rGrUrGrGrArUrGrArGrGrCrArGrGrGrCrCrArGrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 954 | rArGrArGrArUrUrGrGrUrGrGrArUrGrArGrGrCrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 955 | rGrGrCrUrUrArGrArGrArUrUrGrGrUrGrGrArUrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 956 | rGrGrGrUrUrUrGrGrCrUrUrArGrArGrArUrUrGrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 957 | rUrArGrGrGrGrUrUrUrGrGrCrUrUrArGrArGrArUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 958 | rUrArArGrCrCrArArArCrCrCrCrUrArArArCrUrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 959 | rGrArCrUrCrCrUrGrArGrUrUrUrArGrGrGrGrUrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 960 | ArCrGrUrUrGrArCrUrCrCrUrGrArGrUrUrUrArGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 961 | rCrArCrGrUrUrGrArCrUrCrCrUrGrArGrUrUrUrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 962 | ArCrArCrGrUrUrGrArCrUrCrCrUrGrArGrUrUrUrGrUrUrUrUrArGrArGrCrU |
| | ArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 963 | rGrCrArArGrCrCrUrUrGrCrUrArGrArCrArGrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 964 | rGrCrUrArArCrCrUrGrGrCrUrGrUrCrUrArGrCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 965 | rUrGrArCrArGrGrGrCrArArArGrGrCrUrArArCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 966 | rArUrUrCrGrGrGrGrGrUrGrArCrArGrGrGrCrArArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 967 | rGrUrCrArUrGrArUrUrCrGrGrGrGrGrUrGrArCrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 968 | rGrGrUrCrArUrGrArUrUrCrGrGrGrGrGrUrGrArCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 969 | rCrUrGrGrGrUrGrGrGrUrCrArUrGrArUrUrCrGrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 970 | rArCrUrGrGrGrUrGrGrGrUrCrArUrGrArUrUrCrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 971 | rCrArCrUrGrGrGrUrGrGrGrUrCrArUrGrArUrUrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 972 | rArCrArCrUrGrGrGrUrGrGrGrUrCrArUrGrArUrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 973 | rGrArCrCrCrArCrCrCrArGrUrGrUrCrUrUrUrCrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 974 | rCrCrArCrCrCrArGrUrGrUrCrUrUrUrCrGrArGrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 975 | rCrArCrCrCrArGrUrGrUrCrUrUrUrCrGrArGrGrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 976 | rCrArCrCrUrCrGrArArArGrArCrArCrUrGrGrGrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 977 | rArArCrCrCrArCrCrUrCrGrArArArGrArCrArCrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 978 | rArArArCrCrCrArCrCrUrCrGrArArArGrArCrArCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 979 | rGrUrUrUrGrUrArCrCrUrUrCrCrUrUrArArGrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 980 | rUrArCrCrUrUrCrCrUrUrArArGrCrCrArGrGrArArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 981 | rArCrCrUrUrCrCrUrUrArArGrCrCrArGrGrArArArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 982 | rUrCrCrCrUrUrUrCrCrUrGrGrCrUrUrArArGrGrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 983 | rUrGrArArUrCrCrCrUrUrUrCrCrUrGrGrCrUrUrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 984 | rUrArArGrCrCrArGrGrArArArGrGrGrArUrUrCrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 985 | rArGrGrArArArGrGrGrArUrUrCrArUrGrGrCrGrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 986 | rGrArCrGrCrCrArUrGrArArUrCrCrCrUrUrUrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 987 | rUrCrArUrGrGrCrGrUrCrGrGrArArArUrGrArUrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 988 | rArArArUrGrArUrCrUrGrGrCrUrGrArArUrCrCrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 989 | rUrGrArUrCrUrGrGrCrUrGrArArUrCrCrGrUrGrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 990 | rGrUrUrUrGrGrUrCrUrCrGrGrUrGrCrCrArCrCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 991 | rUrUrGrGrUrGrArArUrGrArGrUrUrUrGrGrUrCrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 992 | rCrArUrCrArUrUrUrGrGrUrGrArArUrGrArGrUrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 993 | rCrArArArUrGrArUrGrCrCrArCrUrUrCrCrCrArGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 994 | rCrUrCrUrGrGrGrArArGrUrGrGrCrArUrCrArUrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 995 | rCrArGrGrCrUrCrUrGrCrCrUrCrUrGrGrGrArArGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 996 | rArGrArGrGrCrArGrArGrCrCrUrGrArGrUrCrArCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 997 | rArGrUrGrArCrUrCrArGrGrCrUrCrUrGrCrCrUrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 998 | rUrUrArArGrGrGrUrGrArCrCrArGrUrGrArCrUrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 999 | rCrArGrGrCrArCrUrUrArArUrArArArUrArUrUrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1000 | rUrArUrUrArArGrUrGrCrCrUrGrArGrArCrArCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1001 | rCrCrUrGrArGrArCrArCrCrCrGrGrUrUrArCrCrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1002 | rCrCrArArGrGrUrArArCrCrGrGrGrUrGrUrCrUrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1003 | rArCrCrCrGrGrUrUrArCrCrUrUrGrGrCrCrGrUrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1004 | rUrCrCrUrCrArCrGrGrCrCrArArGrGrUrArArCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1005 | rGrUrCrCrUrCrArCrGrGrCrCrArArGrGrUrArArCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1006 | rArCrCrUrUrGrGrCrCrGrUrGrArGrGrArCrArCrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1007 | rGrCrCrArCrGrUrGrUrCrCrUrCrArCrGrGrCrCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1008 | rGrUrGrCrArGrGrCrCrArCrGrUrGrUrCrCrUrCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1009 | rArGrGrArCrArCrGrUrGrGrCrCrUrGrCrArCrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1010 | rArCrGrUrGrGrCrCrUrGrCrArCrCrCrArGrGrUrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1011 | rUrGrCrArCrCrCrArGrGrUrGrUrGrGrCrUrGrUrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1012 | rUrGrArCrArGrCrCrArCrArCrCrUrGrGrGrUrGrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1013 | rGrUrGrUrCrCrUrGrArCrArGrCrCrArCrArCrCrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1014 | rGrGrUrGrUrCrCrUrGrArCrArGrCrCrArCrArCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1015 | rUrGrGrCrUrGrUrCrArGrGrArCrArCrCrArGrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1016 | rCrGrGrGrArGrGrArUrGrGrGrCrArCrCrArGrGrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1017 | rGrGrGrUrCrGrGrGrArGrGrArUrGrGrGrCrArCrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1018 | rUrGrGrGrUrArGrGrGrGrUrCrGrGrGrArGrGrArUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1019 | rGrGrArArGrUrGrGrGrUrArGrGrGrGrUrCrGrGrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1020 | rCrCrUrArCrCrCrArCrUrUrCrCrArUrUrCrCrCrGrGrUrUrUrUrArGrArGrCrUr |
| | ArGrArArArUrArGrCrArArGrUrUrArArArArUrArArGrGrCrUrArGrUrCrCrG |
| | rUrUrArUrCrArArCrUrUrGrArArArArArGrUrGrGrCrArCrCrGrArGrUrCrGrG |
| | rUrGrCrUrUrUrU |
| 1021 | rArCrGrGrGrArArUrGrGrArArGrUrGrGrGrUrArGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1022 | rCrArCrGrGrGrArArUrGrGrArArGrUrGrGrGrUrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1023 | rCrCrArCrGrGrGrArArUrGrGrArArGrUrGrGrGrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1024 | rGrArGrArCrCrArCrGrGrGrArArUrGrGrArArGrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1025 | rGrGrArGrArCrCrArCrGrGrGrArArUrGrGrArArGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1026 | rGrUrGrCrArArGrGrArGrArCrCrArCrGrGrGrArArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1027 | rArGrArArArGrUrGrCrArArGrGrArGrArCrCrArCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1028 | rGrArGrArArArGrUrGrCrArArGrGrArGrArCrCrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1029 | rCrUrCrUrGrArArCrUrGrArGrArArArGrUrGrCrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1030 | rArGrUrUrGrUrArCrArCrUrGrUrGrUrArCrArUrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1031 | rUrGrUrUrUrUrCrUrGrUrCrGrUrGrUrGrUrGrUrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1032 | rUrUrCrUrGrUrCrGrUrGrUrGrUrGrUrUrGrGrGrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1033 | rUrCrUrGrUrCrGrUrGrUrGrUrGrUrUrGrGrGrArUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1034 | rGrUrGrUrGrUrUrGrGrGrArUrGrGrGrArUrCrCrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1035 | rArCrArCrGrGrGrCrUrUrUrCrCrCrUrGrGrCrCrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1036 | rGrArCrArCrGrGrGrCrUrUrUrCrCrCrUrGrGrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1037 | rUrCrArUrUrGrArCrArCrGrGrGrCrUrUrUrCrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1038 | rArArGrCrCrCrGrUrGrUrCrArArUrGrArArUrGrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1039 | rArGrCrCrCrGrUrGrUrCrArArUrGrArArUrGrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1040 | rGrCrCrCrGrUrGrUrCrArArUrGrArArUrGrCrCrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1041 | rUrCrCrCrCrGrGrCrArUrUrCrArUrUrGrArCrArCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1042 | rGrUrCrCrCrCrGrGrCrArUrUrCrArUrUrGrArCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1043 | rArArUrGrArArUrGrCrCrGrGrGrGrArCrArGrArGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1044 | rArUrGrArArUrGrCrCrGrGrGrGrArCrArGrArGrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1045 | rUrGrCrCrGrGrGrGrArCrArGrArGrArGrGrGrGrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1046 | rArArCrCrUrGrCrCrCrCrUrCrUrCrUrGrUrCrCrCrGrUrUrUrUrArGrArGrCrUr |
| | ArGrArArArUrArGrCrArArGrUrUrArArArArUrArArGrGrCrUrArGrUrCrCrG |
| | rUrUrArUrCrArArCrUrUrGrArArArArArGrUrGrGrCrArCrCrGrArGrUrCrGrG |
| | rUrGrCrUrUrUrU |
| 1047 | rArCrArGrArGrArGrGrGrGrCrArGrGrUrUrGrArCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1048 | rCrArGrArGrArGrGrGrGrCrArGrGrUrUrGrArCrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1049 | rCrGrArUrCrArCrGrGrCrUrUrUrGrArArGrUrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1050 | rGrUrUrCrUrCrGrArUrArUrUrCrArCrGrArUrCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1051 | rGrGrCrGrGrArCrArUrArArArGrArCrGrArCrArArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1052 | rArArGrUrGrGrArArGrCrArCrUrArGrGrUrGrGrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1053 | rUrArGrArArGrUrGrGrArArGrCrArCrUrArGrGrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1054 | rArUrArGrArArGrUrGrGrArArGrCrArCrUrArGrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1055 | rUrGrCrArUrArGrArArGrUrGrGrArArGrCrArCrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1056 | rUrGrGrArGrGrCrArUrUrUrGrCrArUrArGrArArGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1057 | rUrGrGrGrGrArArGrUrGrArArUrGrGrCrUrUrGrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1058 | rGrArUrUrGrGrGrGrArArGrUrGrArArUrGrGrCrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1059 | rGrArCrArArGrArUrUrGrGrGrGrArArGrUrGrArArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1060 | rUrUrCrCrCrCrArArUrCrUrUrGrUrCrGrUrUrGrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1061 | rUrCrCrCrCrArArUrCrUrUrGrUrCrGrUrUrGrArUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1062 | rArCrCrCrArUrCrArArCrGrArCrArArGrArUrUrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1063 | rUrArCrCrCrArUrCrArArCrGrArCrArArGrArUrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1064 | rGrUrArUrGrUrGrUrUrUrArArArArCrArUrGrCrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1065 | rUrGrUrUrUrArArArArCrArUrGrCrArCrGrGrUrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1066 | rUrArArArArCrArUrGrCrArCrGrGrUrGrArGrGrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1067 | rArArArArCrArUrGrCrArCrGrGrUrGrArGrGrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1068 | rGrGrCrUrGrCrUrGrArUrGrCrArCrCrArUrGrCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1069 | rCrArUrGrGrUrGrCrArUrCrArGrCrArGrCrCrCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1070 | rCrArUrCrArGrCrArGrCrCrCrArUrGrGrCrCrUrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1071 | rCrArUrGrCrCrUrGrGrCrCrArGrArGrGrCrCrArUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1072 | rGrCrCrUrCrUrGrGrCrCrArGrGrCrArUrGrGrCrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1073 | rGrCrCrUrCrGrCrCrArUrGrCrCrUrGrGrCrCrArGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1074 | rCrArUrCrUrCrUrUrArArArArArArUrGrArArUrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1075 | rArArArUrGrArArUrUrUrGrGrCrCrArGrArCrArCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1076 | rUrArUrArGrUrUrGrUrArUrUrUrCrUrGrArArArUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1077 | rCrUrCrCrArGrUrCrUrGrGrArUrCrGrUrUrUrGrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1078 | rUrCrCrArGrUrCrUrGrGrArUrCrGrUrUrUrGrArCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1079 | rCrGrUrCrArArArCrGrArUrCrCrArGrArCrUrGrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1080 | rUrCrCrCrGrUrCrArArArCrGrArUrCrCrArGrArCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1081 | rGrGrArUrCrGrUrUrUrGrArCrGrGrGrArCrUrUrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1082 | rArCrArUrCrArGrUrGrCrArArCrArGrUrArArCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1083 | rGrGrArGrGrCrUrGrUrCrArCrUrGrUrCrUrCrUrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1084 | rUrUrCrArCrGrCrGrGrGrArGrUrCrUrGrArCrGrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1085 | rArUrCrUrUrCrArCrGrCrGrGrGrArGrUrCrUrGrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1086 | rUrCrCrCrGrCrGrUrGrArArGrArUrGrUrCrArCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1087 | rCrCrCrGrCrGrUrGrArArGrArUrGrUrCrArCrArArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1088 | rCrCrCrUrUrGrUrGrArCrArUrCrUrUrCrArCrGrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1089 | rUrCrCrCrUrUrGrUrGrArCrArUrCrUrUrCrArCrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1090 | rGrUrGrArArGrArUrGrUrCrArCrArArGrGrGrArUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1091 | rArGrGrGrArUrUrGrGrCrArArUrUrGrUrCrCrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1092 | rGrGrGrArUrUrGrGrCrArArUrUrGrUrCrCrCrCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1093 | rGrArCrArCrArGrUrGrUrUrUrUrGrUrCrCrCrUrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1094 | rGrGrArCrArCrArGrUrGrUrUrUrUrGrUrCrCrCrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1095 | rGrGrGrArCrArCrArGrUrGrUrUrUrUrGrUrCrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1096 | rCrArCrUrGrUrGrUrCrCrCrCrCrCrCrArGrUrGrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1097 | rArCrUrGrUrGrUrCrCrCrCrCrCrCrArGrUrGrCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1098 | rCrArCrGrGrUrUrCrCrCrUrGrCrArCrUrGrGrGrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1099 | rUrCrArCrGrGrUrUrCrCrCrUrGrCrArCrUrGrGrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1100 | rArUrCrArCrGrGrUrUrCrCrCrUrGrCrArCrUrGrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1101 | rUrArUrCrArCrGrGrUrUrCrCrCrUrGrCrArCrUrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1102 | rUrUrArUrCrArCrGrGrUrUrCrCrCrUrGrCrArCrUrGrUrUrUrUrArGrArGrCrU |
| | ArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1103 | rCrUrUrArUrCrArCrGrGrUrUrCrCrCrUrGrCrArCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1104 | rGrGrArArCrCrGrUrGrArUrArArGrCrCrUrUrUrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1105 | rGrUrGrArUrArArGrCrCrUrUrUrCrUrGrGrUrUrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1106 | rGrArArArCrCrArGrArArArGrGrCrUrUrArUrCrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1107 | rUrArCrGrUrGrCrUrCrCrGrArArArCrCrArGrArArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1108 | rArUrGrCrGrUrCrCrCrUrGrUrArCrArGrArUrArGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1109 | rUrGrCrGrUrCrCrCrUrGrUrArCrArGrArUrArGrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1110 | rGrCrGrUrCrCrCrUrGrUrArCrArGrArUrArGrUrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1111 | rArArArUrCrCrCrCrArCrUrArUrCrUrGrUrArCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1112 | rArArArArUrCrCrCrCrArCrUrArUrCrUrGrUrArCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1113 | rUrArUrGrUrUrUrGrCrArCrUrUrUrGrUrArUrArUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1114 | rCrUrArUrUrUrArUrUrUrUrUrGrCrArArArCrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1115 | rCrUrGrArArCrArArArUrArCrArGrCrArArCrCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1116 | rArCrUrGrArArCrArArArUrArCrArGrCrArArCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1117 | rArUrUrUrGrUrUrCrArGrUrGrArCrUrArUrUrCrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1118 | rUrUrUrGrUrUrCrArGrUrGrArCrUrArUrUrCrUrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1119 | rUrUrGrUrUrCrArGrUrGrArCrUrArUrUrCrUrCrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1120 | rCrUrArUrUrCrUrCrGrGrGrGrCrCrCrUrGrUrGrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1121 | rUrArUrUrCrUrCrGrGrGrGrCrCrCrUrGrUrGrUrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1122 | rArUrUrCrUrCrGrGrGrGrCrCrCrUrGrUrGrUrArGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1123 | rUrUrCrUrCrGrGrGrGrCrCrCrUrGrUrGrUrArGrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1124 | ArGrGrCrArArUrArArCrCrCrCrCrUrArCrArCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1125 | rGrArGrGrCrArArUrArArCrCrCrCrCrUrArCrArCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1126 | rArUrArArArGrArArGrArGrGrCrArUrUrUrCrArGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1127 | rArArUrCrUrUrUrGrUrArCrArUrArArArGrArArGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1128 | rCrArArArGrArUrUrArUrUrUrGrCrArCrGrArArCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1129 | rGrGrArCrUrGrUrGrUrGrCrArArCrGrCrUrUrUrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1130 | rGrArCrUrGrUrGrUrGrCrArArCrGrCrUrUrUrUrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1131 | rGrUrCrCrCrCrGrUrUrGrUrArUrGrUrArUrGrArGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1132 | rArGrCrCrArCrUrCrArUrArCrArUrArCrArArCrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1133 | ArArGrCrCrArCrUrCrArUrArCrArUrArCrArArCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1134 | rGrArArGrCrCrArCrUrCrArUrArCrArUrArCrArArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1135 | rUrUrGrUrArUrGrUrArUrGrArGrUrGrGrCrUrUrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1136 | rUrGrUrArUrGrUrArUrGrArGrUrGrGrCrUrUrCrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1137 | rUrArUrGrArGrUrGrGrCrUrUrCrUrGrGrGrArGrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1138 | rArUrGrArGrUrGrGrCrUrUrCrUrGrGrGrArGrArUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1139 | rUrArCrArArArArArCrCrUrUrCrUrArUrArCrArGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1140 | rUrUrGrArUrCrArCrArGrUrArArGrArCrArUrUrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1141 | rUrGrCrArUrCrGrArGrUrUrUrArGrArCrArArArUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1142 | rUrUrArGrCrGrUrUrGrGrCrCrArCrCrCrGrCrGrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1143 | rArArArUrUrArGrCrGrUrUrGrGrCrCrArCrCrCrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1331 | rUrUrGrArArArUrUrCrGrArArUrUrCrUrGrCrCrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1332 | rGrUrGrCrGrUrUrGrCrUrUrUrGrArGrUrGrGrGrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1421 | rArCrArGrArUrGrArCrArGrArArGrGrArArGrUrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1422 | rUrUrCrCrUrUrCrUrGrUrCrArUrCrUrGrUrUrUrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1423 | rUrCrUrGrUrCrArUrCrUrGrUrUrUrGrUrGrGrArUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1424 | rArUrCrCrArCrArArArCrArGrArUrGrArCrArGrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1425 | rGrUrUrUrGrUrGrGrArUrUrGrGrArCrUrCrCrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1426 | rGrGrArUrUrGrGrArCrUrCrCrCrCrArGrGrArGrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1427 | rGrGrGrArArGrArCrUrCrCrCrArGrArArCrUrCrCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1428 | rGrUrGrUrArUrGrArArUrGrGrArUrGrArGrCrArGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1429 | rArGrUrGrUrArUrGrArArUrGrGrArUrGrArGrCrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1430 | rArUrArUrUrUrGrUrCrGrArGrUrGrUrArUrGrArArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1431 | rCrArCrArArGrCrCrUrArCrGrGrCrArGrArGrCrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1432 | rGrUrArGrGrCrUrUrGrUrGrGrGrArCrArCrUrArCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1433 | rUrArGrGrCrUrUrGrUrGrGrGrArCrArCrUrArCrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1434 | rUrGrUrArGrUrGrUrCrCrCrArCrArArGrCrCrUrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1435 | rArGrGrCrUrUrGrUrGrGrGrArCrArCrUrArCrArGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1436 | rArArArGrArArArUrGrArArGrUrCrUrUrGrArUrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1437 | rGrArArArUrGrArArGrUrCrUrUrGrArUrArCrGrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1438 | rArArUrCrUrCrCrCrUrGrUrCrUrGrUrCrCrUrUrArGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1439 | rUrCrArGrGrUrCrCrUrUrArArGrGrArCrArGrArCrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1440 | ArArArArUrGrArUrGrUrCrArGrGrUrCrCrUrUrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1441 | rCrCrUrCrArArGrUrCrGrUrCrUrArCrCrGrCrCrUrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1442 | rCrCrGrArGrGrCrGrGrUrArGrArCrGrArCrUrUrGrGrUrUrUrUrArGrArGrCrU |
| | rArGrArArArUrArGrCrArArGrUrUrArArArArUrArArGrGrCrUrArGrUrCrCr |
| | GrUrUrArUrCrArArCrUrUrGrArArArArArGrUrGrGrCrArCrCrGrArGrUrCrGr |
| | GrUrGrCrUrUrUrU |
| 1443 | rUrUrCrUrCrUrArArUrGrArArGrUrArArCrArUrUrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1444 | rCrArGrArArArArGrCrArArCrUrGrGrCrArGrGrArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1445 | rGrGrGrArCrArGrArArArArGrCrArArCrUrGrGrCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1446 | rUrArUrGrGrGrGrArCrArGrArArArArGrCrArArCrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1447 | rUrUrUrUrGrUrArUrCrUrArUrUrCrUrArArArArArGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1448 | rUrGrUrArUrCrUrArUrUrCrUrArArArArArUrGrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1421 | rArCrArGrArUrGrArCrArGrArArGrGrArArGrUrGrGrUrUrUrUrArGrArGrCr |
| | UrArGrArArArUrArGrCrArArGrUrUrArArArArUrArArGrGrCrUrArGrUrCrC |
| | rGrUrUrArUrCrArArCrUrUrGrArArArArArGrUrGrGrCrArCrCrGrArGrUrCrG |
| | rGrUrGrCrUrUrUrU |
| 1459 | mC*mA*mG*rUrGrCrCrCrArUrGrCrArArUrGrGrCrUrUrGrUrUrUrUrArGrA |
| | mGmCmUmAmGmAmAmAmUmAmGmCrArArGrUrUrArArArArUrArArGr |
| | GrCrUrArGrUrCrCrGrUrUrArUrCrAmAmCmUmUmGmAmAmAmAmAmG |
| | mUmGmGmCmAmCmCmGmAmGmUmCmGmGmUmGmCmU*mU*mU*m |
| | U |
| 1460 | mC*mA*mG*rUrGrCrCrCrArUrGrCrArArUrGrGrCrUrUrGrUrUrUrUrArGrA |
| | mGmCmUmAmGmAmAmAmUmAmGmCrArArGrUrUrArArArArUrArArGr |
| | GrCrUrArGrUrCrCrGrUrUrArUrCrAmAmCmUmUmGmAmAmAmAmAmG |
| | mUmGmGmCmAmCmCmGmAmGmUmCmGmGmUmGmCmU*mU*mU*m |
| | U |
| 1463 | mG*mA*mG*rCrArArCrArCrArGrArGrCrCrCrArGrGrGrGrUrUrUrUrArGrA |
| | mGmCmUmAmGmAmAmAmUmAmGmCrArArGrUrUrArArArArUrArArGr |
| | GrCrUrArGrUrCrCrGrUrUrArUrCrAmAmCmUmUmGmAmAmAmAmAmG |
| | mUmGmGmCmAmCmCmGmAmGmUmCmGmGmUmGmCmU*mU*mU*m |
| | U |
| 1465 | mU*mU*mG*rArArArUrUrCrGrArArUrUrCrUrGrCrCrUrGrUrUrUrUrArGrA |
| | mGmCmUmAmGmAmAmAmUmAmGmCrArArGrUrUrArArArArUrArArGr |
| | GrCrUrArGrUrCrCrGrUrUrArUrCrAmAmCmUmUmGmAmAmAmAmAmG |
| | mUmGmGmCmAmCmCmGmAmGmUmCmGmGmUmGmCmU*mU*mU*m |
| | U |
| 1467 | mG*mU*mG*rCrGrUrUrGrCrUrUrUrGrArGrUrGrGrGrUrGrUrUrUrUrArGrA |
| | mGmCmUmAmGmAmAmAmUmAmGmCrArArGrUrUrArArArArUrArArGr |
| | GrCrUrArGrUrCrCrGrUrUrArUrCrAmAmCmUmUmGmAmAmAmAmAmG |
| | mUmGmGmCmAmCmCmGmAmGmUmCmGmGmUmGmCmU*mU*mU*m |
| | U |
| 1469 | mC*mA*mG*rTrGrCrCrCrArTrGrCrArArTrGrGrCrTrTrGrUrUrUrUrArGrAm |
| | GmCmUmAmGmAmAmAmUmAmGmCrArArGrUrUrArArArArUrArArGrGr |
| | CrUrArGrUrCrCrGrUrUrArUrCrAmAmCmUmUmGmAmAmAmAmAmGmU |
| | mGmGmCmAmCmCmGmAmGmUmCmGmGmUmGmCmU*mU*mU*mU |
| 1471 | mA*mT*mG*rArGrTrGrGrCrTrTrCrTrGrGrGrArGrArTrGrUrUrUrUrArGrAm |
| | GmCmUmAmGmAmAmAmUmAmGmCrArArGrUrUrArArArArUrArArGrGr |
| | CrUrArGrUrCrCrGrUrUrArUrCrAmAmCmUmUmGmAmAmAmAmAmGmU |
| | mGmGmCmAmCmCmGmAmGmUmCmGmGmUmGmCmU*mU*mU*mU |
| 1472 | mU*mU*mG*rArArArUrUrCrGrArArUrUrCrUrGrCrCrUrGrUrUrUrUrArGrA |
| | mGmCmUmAmGmAmAmAmUmAmGmCrArArGrUrUrArArArArUrArArGr |
| | GrCrUrArGrUrCrCrGrUrUrArUrCrAmAmCmUmUmGmAmAmAmAmAmG |
| | mUmGmGmCmAmCmCmGmAmGmUmCmGmGmUmGmCmU*mU*mU*m |
| | U |
| 1473 | mG*mU*mG*rCrGrUrUrGrCrUrUrUrGrArGrUrGrGrGrUrGrUrUrUrUrArGrA |
| | mGmCmUmAmGmAmAmAmUmAmGmCrArArGrUrUrArArArArUrArArGr |
| | GrCrUrArGrUrCrCrGrUrUrArUrCrAmAmCmUmUmGmAmAmAmAmAmG |
| | mUmGmGmCmAmCmCmGmAmGmUmCmGmGmUmGmCmU*mU*mU*m |
| | U |
| 1476 | mA*mG*mC*rUrCrUrGrGrCrArGrGrCrArArUrGrCrUrUrGrUrUrUrUrArGrA |
| | mGmCmUmAmGmAmAmAmUmAmGmCrArArGrUrUrArArArArUrArArGr |
| | GrCrUrArGrUrCrCrGrUrUrArUrCrAmAmCmUmUmGmAmAmAmAmAmG |
| | mUmGmGmCmAmCmCmGmAmGmUmCmGmGmUmGmCmU*mU*mU*m |
| | U |
| 1477 | mA*mU*mA*rUrArArArArCrArArArGrCrUrCrUrGrGrCrGrUrUrUrUrArGrA |
| | mGmCmUmAmGmAmAmAmUmAmGmCrArArGrUrUrArArArArUrArArGr |
| | GrCrUrArGrUrCrCrGrUrUrArUrCrAmAmCmUmUmGmAmAmAmAmAmG |
| | mUmGmGmCmAmCmCmGmAmGmUmCmGmGmUmGmCmU*mU*mU*m |
| | U |
| 1478 | mA*mA*mA*rUrArUrArUrArArArArCrArArArGrCrUrCrGrUrUrUrUrArGrA |
| | mGmCmUmAmGmAmAmAmUmAmGmCrArArGrUrUrArArArArUrArArGr |
| | GrCrUrArGrUrCrCrGrUrUrArUrCrAmAmCmUmUmGmAmAmAmAmAmG |
| | mUmGmGmCmAmCmCmGmAmGmUmCmGmGmUmGmCmU*mU*mU*m |
| | U |
| 1479 | mA*mC*mG*rUrGrGrCrGrUrGrArArCrArUrCrUrGrCrCrGrUrUrUrUrArGrA |
| | mGmCmUmAmGmAmAmAmUmAmGmCrArArGrUrUrArArArArUrArArGr |
| | GrCrUrArGrUrCrCrGrUrUrArUrCrAmAmCmUmUmGmAmAmAmAmAmG |
| | mUmGmGmCmAmCmCmGmAmGmUmCmGmGmUmGmCmU*mU*mU*m |
| | U |
| 1449 | mA*mA*mU*rCrUrUrUrGrUrArCrArUrArArArGrArArGrGrUrUrUrUrArGrA |
| | mGmCmUmAmGmAmAmAmUmAmGmCrArArGrUrUrArArArArUrArArGr |
| | GrCrUrArGrUrCrCrGrUrUrArUrCrAmAmCmUmUmGmAmAmAmAmAmG |
| | mUmGmGmCmAmCmCmGmAmGmUmCmGmGmUmGmCmU*mU*mU*m |
| | U |
| 1450 | mU*mA*mC*rArArArArArCrCrUrUrCrUrArUrArCrArGrGrUrUrUrUrArGrA |
| | mGmCmUmAmGmAmAmAmUmAmGmCrArArGrUrUrArArArArUrArArGr |
| | GrCrUrArGrUrCrCrGrUrUrArUrCrAmAmCmUmUmGmAmAmAmAmAmG |
| | mUmGmGmCmAmCmCmGmAmGmUmCmGmGmUmGmCmU*mU*mU*m |
| | U |

All bases are in upper case Lowercase “r” represents RNA, 2′-hydroxy; bases not modified by an “r” are DNA
All bases are linked via standard phosphodiester bonds except as noted:
“*” represents phosphorothioate modification

TABLE 7
LDLR Target Sequences for AsCas12a WT

| SEQ ID NO | Target Sequence (DNA) |
|---|---|
| 549 | AATAAAACAAGGCCGGCGAGG |
| 550 | TTCATCTGGGAGGCAGAACAG |
| 551 | GTTTCTTCCTTTCCTCGTGAA |
| 552 | TTCCTTTCCTCGTGAAGGATA |
| 553 | TCTTATCCTTCACGAGGAAAG |
| 554 | AGTTTCTCTCCACCGTGACAC |
| 555 | AGGATTGTGTCACGGTGGAGA |
| 556 | TCTCCACCGTGACACAATCCT |
| 557 | ATCTTCCATGTTTGAGGATTG |
| 558 | AACACACAACAGCAGATGGCA |
| 559 | CCTCTTCTATGCAAGCCTTGC |
| 560 | GAGGTGGGTTTGTACCTTCCT |
| 561 | CTGGCTTAAGGAAGGTACAAA |
| 562 | CGACGCCATGAATCCCTTTCC |
| 563 | GTCTCGGTGCCACCACGGATT |
| 564 | GTGAATGAGTTTGGTCTCGGT |
| 565 | TTAAGTGCCTGAGACACCCGG |
| 566 | TCAGTTCAGAGTTGTACACTG |
| 567 | CACTGTTTTCTGTCGTGTGTG |
| 568 | AAGTCCCGGTCAACCTGCCCC |
| 569 | TGTCCGCCCACCTAGTGCTTC |
| 570 | CATAGAAGTGGAAGCACTAGG |
| 571 | AACACATACCCATCAACGACA |
| 572 | AAACATGCACGGTGAGGCCGG |
| 573 | ACGGGACTTCAGGTTCTTTCT |
| 574 | AGAAAGAACCTGAAGTCCCGT |
| 575 | TGAAATCGCCGTGTTACTGTT |
| 576 | TCCCTGGGGACAATTGCCAAT |
| 577 | CGTGCTCCGAAACCAGAAAGG |
| 578 | TGGTTTCGGAGCACGTAAATG |
| 579 | GGAGCACGTAAATGCGTCCCT |
| 580 | AACCAATATACAAAGTGCAAA |
| 581 | CACTTTGTATATTGGTTGAAA |
| 582 | TATATTGGTTGAAACTGTTAT |
| 583 | TTTTTGCAAACCCTGGTTGCT |
| 584 | CAAACCCTGGTTGCTGTATTT |
| 585 | AGAGGCAATAACCCCCTACAC |
| 586 | TACATAAAGAAGAGGCATTTC |
| 587 | TGTACAAAGATTATTTGCACG |
| 588 | CACGAACTGGACTGTGTGCAA |
| 589 | GGAGAATGATGTCCCCGTTGT |
| 590 | AACCACTGTATAGAAGGTTTT |
| 591 | TAGCCTGAATGTCTTACTGTG |
| 592 | ATTGATCACAGTAAGACATTC |
| 593 | AGAAATTTAATTGATCACAGT |
| 594 | GACAAATTGGTTCATTTAAGA |
| 595 | TTAAATGAACCAATTTGTCTA |
| 596 | TCTAAACTCGATGCACGTTCT |
| 597 | TGGATTGGACTCCCCAGGAGA |
| 598 | TCAAGTCCCCTGCTCATCCAT |
| 599 | TCGAGTGTATGAATGGATGAG |
| 600 | TTATATCCCCTGTAGTGTCCC |
| 601 | ATTTCTTATATCCCCTGTAGT |
| 602 | TTCAGGGGGAGTTTCTCTAAT |
| 603 | TGGTGCAGTCACCTGCCAGCA |
| 604 | TGGTGGTGGCGCTTTCTGCTG |
| 605 | CCTCCTATGAGTTTCTGGCTC |
| 606 | TGGACCTAACCACCCCAGTGT |
| 1319 | TTGTCTGGGGACAGAAAAGGC |
| 1320 | GCTTCCAAGGAAATGAAGATG |
| 1321 | AATGGGCTGATCTCAGGCCTG |
| 1322 | AAAGCAGTTCCCACCCACTCA |
| 1323 | AGTGGGTGGGAACTGCTTTGA |

TABLE 8
LDLR Targeting Sequences for AsCas12a WT

| SEQ | |
|---|---|
| ID NO | gDNA targeting Domain (RNA) |
| 607 | rArArUrArArArArCrArArGrGrCrCrGrGrCrGrArGrG |
| 608 | rUrUrCrArUrCrUrGrGrGrArGrGrCrArGrArArCrArG |
| 609 | rGrUrUrUrCrUrUrCrCrUrUrUrCrCrUrCrGrUrGrArA |
| 610 | rUrUrCrCrUrUrUrCrCrUrCrGrUrGrArArGrGrArUrA |
| 611 | rUrCrUrUrArUrCrCrUrUrCrArCrGrArGrGrArArArG |
| 612 | rArGrUrUrUrCrUrCrUrCrCrArCrCrGrUrGrArCrArC |
| 613 | rArGrGrArUrUrGrUrGrUrCrArCrGrGrUrGrGrArGrA |
| 614 | rUrCrUrCrCrArCrCrGrUrGrArCrArCrArArUrCrCrU |
| 615 | rArUrCrUrUrCrCrArUrGrUrUrUrGrArGrGrArUrUrG |
| 616 | rArArCrArCrArCrArArCrArGrCrArGrArUrGrGrCrA |
| 617 | rCrCrUrCrUrUrCrUrArUrGrCrArArGrCrCrUrUrGrC |
| 618 | rGrArGrGrUrGrGrGrUrUrUrGrUrArCrCrUrUrCrCrU |
| 619 | rCrUrGrGrCrUrUrArArGrGrArArGrGrUrArCrArArA |
| 620 | rCrGrArCrGrCrCrArUrGrArArUrCrCrCrUrUrUrCrC |
| 621 | rGrUrCrUrCrGrGrUrGrCrCrArCrCrArCrGrGrArUrU |
| 622 | rGrUrGrArArUrGrArGrUrUrUrGrGrUrCrUrCrGrGrU |
| 623 | rUrUrArArGrUrGrCrCrUrGrArGrArCrArCrCrCrGrG |
| 624 | rUrCrArGrUrUrCrArGrArGrUrUrGrUrArCrArCrUrG |
| 625 | rCrArCrUrGrUrUrUrUrCrUrGrUrCrGrUrGrUrGrUrG |
| 626 | rArArGrUrCrCrCrGrGrUrCrArArCrCrUrGrCrCrCrC |
| 627 | rUrGrUrCrCrGrCrCrCrArCrCrUrArGrUrGrCrUrUrC |
| 628 | rCrArUrArGrArArGrUrGrGrArArGrCrArCrUrArGrG |
| 629 | rArArCrArCrArUrArCrCrCrArUrCrArArCrGrArCrA |
| 630 | rArArArCrArUrGrCrArCrGrGrUrGrArGrGrCrCrGrG |
| 631 | rArCrGrGrGrArCrUrUrCrArGrGrUrUrCrUrUrUrCrU |
| 632 | rArGrArArArGrArArCrCrUrGrArArGrUrCrCrCrGrU |
| 633 | rUrGrArArArUrCrGrCrCrGrUrGrUrUrArCrUrGrUrU |
| 634 | rUrCrCrCrUrGrGrGrGrArCrArArUrUrGrCrCrArArU |
| 635 | rCrGrUrGrCrUrCrCrGrArArArCrCrArGrArArArGrG |
| 636 | rUrGrGrUrUrUrCrGrGrArGrCrArCrGrUrArArArUrG |
| 637 | rGrGrArGrCrArCrGrUrArArArUrGrCrGrUrCrCrCrU |
| 638 | rArArCrCrArArUrArUrArCrArArArGrUrGrCrArArA |
| 639 | rCrArCrUrUrUrGrUrArUrArUrUrGrGrUrUrGrArArA |
| 640 | rUrArUrArUrUrGrGrUrUrGrArArArCrUrGrUrUrArU |
| 641 | rUrUrUrUrUrGrCrArArArCrCrCrUrGrGrUrUrGrCrU |
| 642 | rCrArArArCrCrCrUrGrGrUrUrGrCrUrGrUrArUrUrU |
| 643 | rArGrArGrGrCrArArUrArArCrCrCrCrCrUrArCrArC |
| 644 | rUrArCrArUrArArArGrArArGrArGrGrCrArUrUrUrC |
| 645 | rUrGrUrArCrArArArGrArUrUrArUrUrUrGrCrArCrG |
| 646 | rCrArCrGrArArCrUrGrGrArCrUrGrUrGrUrGrCrArA |
| 647 | rGrGrArGrArArUrGrArUrGrUrCrCrCrCrGrUrUrGrU |
| 648 | rArArCrCrArCrUrGrUrArUrArGrArArGrGrUrUrUrU |
| 649 | rUrArGrCrCrUrGrArArUrGrUrCrUrUrArCrUrGrUrG |
| 650 | rArUrUrGrArUrCrArCrArGrUrArArGrArCrArUrUrC |
| 651 | rArGrArArArUrUrUrArArUrUrGrArUrCrArCrArGrU |
| 652 | rGrArCrArArArUrUrGrGrUrUrCrArUrUrUrArArGrA |
| 653 | rUrUrArArArUrGrArArCrCrArArUrUrUrGrUrCrUrA |
| 654 | rUrCrUrArArArCrUrCrGrArUrGrCrArCrGrUrUrCrU |
| 655 | rUrGrGrArUrUrGrGrArCrUrCrCrCrCrArGrGrArGrA |
| 656 | rUrCrArArGrUrCrCrCrCrUrGrCrUrCrArUrCrCrArU |
| 657 | rUrCrGrArGrUrGrUrArUrGrArArUrGrGrArUrGrArG |
| 658 | rUrUrArUrArUrCrCrCrCrUrGrUrArGrUrGrUrCrCrC |
| 659 | rArUrUrUrCrUrUrArUrArUrCrCrCrCrUrGrUrArGrU |
| 660 | rUrUrCrArGrGrGrGrGrArGrUrUrUrCrUrCrUrArArU |
| 661 | rUrGrGrUrGrCrArGrUrCrArCrCrUrGrCrCrArGrCrA |
| 662 | rUrGrGrUrGrGrUrGrGrCrGrCrUrUrUrCrUrGrCrUrG |
| 663 | rCrCrUrCrCrUrArUrGrArGrUrUrUrCrUrGrGrCrUrC |
| 664 | rUrGrGrArCrCrUrArArCrCrArCrCrCrCrArGrUrGrU |
| 1326 | rUrUrGrUrCrUrGrGrGrGrArCrArGrArArArArGrGrC |
| 1327 | rGrCrUrUrCrCrArArGrGrArArArUrGrArArGrArUrG |
| 1328 | rArArUrGrGrGrCrUrGrArUrCrUrCrArGrGrCrCrUrG |
| 1329 | rArArArGrCrArGrUrUrCrCrCrArCrCrCrArCrUrCrA |
| 1330 | rArGrUrGrGrGrUrGrGrGrArArCrUrGrCrUrUrUrGrA |

All bases are in upper case Lowercase represents RNA,
2′-hydroxy; bases not modified by an “r” are DNA
All bases are linked via standard phosphodiester bonds

TABLE 9
gRNA sequences for targeting LDLR with AsCas12a (WT)

| SEQ ID NO | gRNA Sequence |
|---|---|
| 1144 | rUrArArUrUrUrCrUrArCrUrCrUrUrGrUrArGrArUrArArUrArArArArCrArArG |
| | rGrCrCrGrGrCrGrArGrG |
| 1145 | rUrArArUrUrUrCrUrArCrUrCrUrUrGrUrArGrArUrUrUrCrArUrCrUrGrGrGrA |
| | rGrGrCrArGrArArCrArG |
| 1146 | rUrArArUrUrUrCrUrArCrUrCrUrUrGrUrArGrArUrGrUrUrUrCrUrUrCrCrUrU |
| | rUrCrCrUrCrGrUrGrArA |
| 1147 | rUrArArUrUrUrCrUrArCrUrCrUrUrGrUrArGrArUrUrUrCrCrUrUrUrCrCrUrC |
| | rGrUrGrArArGrGrArUrA |
| 1148 | rUrArArUrUrUrCrUrArCrUrCrUrUrGrUrArGrArUrUrCrUrUrArUrCrCrUrUrC |
| | rArCrGrArGrGrArArArG |
| 1149 | rUrArArUrUrUrCrUrArCrUrCrUrUrGrUrArGrArUrArGrUrUrUrCrUrCrUrCrC |
| | rArCrCrGrUrGrArCrArC |
| 1150 | rUrArArUrUrUrCrUrArCrUrCrUrUrGrUrArGrArUrArGrGrArUrUrGrUrGrUrC |
| | rArCrGrGrUrGrGrArGrA |
| 1151 | rUrArArUrUrUrCrUrArCrUrCrUrUrGrUrArGrArUrUrCrUrCrCrArCrCrGrUrG |
| | rArCrArCrArArUrCrCrU |
| 1152 | rUrArArUrUrUrCrUrArCrUrCrUrUrGrUrArGrArUrArUrCrUrUrCrCrArUrGrU |
| | rUrUrGrArGrGrArUrUrG |
| 1153 | rUrArArUrUrUrCrUrArCrUrCrUrUrGrUrArGrArUrArArCrArCrArCrArArCrA |
| | rGrCrArGrArUrGrGrCrA |
| 1154 | rUrArArUrUrUrCrUrArCrUrCrUrUrGrUrArGrArUrCrCrUrCrUrUrCrUrArUrG |
| | rCrArArGrCrCrUrUrGrC |
| 1155 | rUrArArUrUrUrCrUrArCrUrCrUrUrGrUrArGrArUrGrArGrGrUrGrGrGrUrUrU |
| | rGrUrArCrCrUrUrCrCrU |
| 1156 | rUrArArUrUrUrCrUrArCrUrCrUrUrGrUrArGrArUrCrUrGrGrCrUrUrArArGrG |
| | rArArGrGrUrArCrArArA |
| 1157 | rUrArArUrUrUrCrUrArCrUrCrUrUrGrUrArGrArUrCrGrArCrGrCrCrArUrGrA |
| | rArUrCrCrCrUrUrUrCrC |
| 1158 | rUrArArUrUrUrCrUrArCrUrCrUrUrGrUrArGrArUrGrUrCrUrCrGrGrUrGrCrC |
| | rArCrCrArCrGrGrArUrU |
| 1159 | rUrArArUrUrUrCrUrArCrUrCrUrUrGrUrArGrArUrGrUrGrArArUrGrArGrUrU |
| | rUrGrGrUrCrUrCrGrGrU |
| 1160 | rUrArArUrUrUrCrUrArCrUrCrUrUrGrUrArGrArUrUrUrArArGrUrGrCrCrUrG |
| | rArGrArCrArCrCrCrGrG |
| 1161 | UrArArUrUrUrCrUrArCrUrCrUrUrGrUrArGrArUrUrCrArGrUrUrCrArGrArG |
| | rUrUrGrUrArCrArCrUrG |
| 1162 | rUrArArUrUrUrCrUrArCrUrCrUrUrGrUrArGrArUrCrArCrUrGrUrUrUrUrCrU |
| | rGrUrCrGrUrGrUrGrUrG |
| 1163 | rUrArArUrUrUrCrUrArCrUrCrUrUrGrUrArGrArUrArArGrUrCrCrCrGrGrUrC |
| | rArArCrCrUrGrCrCrCrC |
| 1164 | rUrArArUrUrUrCrUrArCrUrCrUrUrGrUrArGrArUrUrGrUrCrCrGrCrCrCrArC |
| | rCrUrArGrUrGrCrUrUrC |
| 1165 | rUrArArUrUrUrCrUrArCrUrCrUrUrGrUrArGrArUrCrArUrArGrArArGrUrGrG |
| | rArArGrCrArCrUrArGrG |
| 1166 | rUrArArUrUrUrCrUrArCrUrCrUrUrGrUrArGrArUrArArCrArCrArUrArCrCrC |
| | rArUrCrArArCrGrArCrA |
| 1167 | rUrArArUrUrUrCrUrArCrUrCrUrUrGrUrArGrArUrArArArCrArUrGrCrArCrG |
| | rGrUrGrArGrGrCrCrGrG |
| 1168 | rUrArArUrUrUrCrUrArCrUrCrUrUrGrUrArGrArUrArCrGrGrGrArCrUrUrCrA |
| | rGrGrUrUrCrUrUrUrCrU |
| 1169 | rUrArArUrUrUrCrUrArCrUrCrUrUrGrUrArGrArUrArGrArArArGrArArCrCrU |
| | rGrArArGrUrCrCrCrGrU |
| 1170 | rUrArArUrUrUrCrUrArCrUrCrUrUrGrUrArGrArUrUrGrArArArUrCrGrCrCrG |
| | rUrGrUrUrArCrUrGrUrU |
| 1171 | rUrArArUrUrUrCrUrArCrUrCrUrUrGrUrArGrArUrUrCrCrCrUrGrGrGrGrArC |
| | rArArUrUrGrCrCrArArU |
| 1172 | rUrArArUrUrUrCrUrArCrUrCrUrUrGrUrArGrArUrCrGrUrGrCrUrCrCrGrArA |
| | rArCrCrArGrArArArGrG |
| 1173 | rUrArArUrUrUrCrUrArCrUrCrUrUrGrUrArGrArUrUrGrGrUrUrUrCrGrGrArG |
| | rCrArCrGrUrArArArUrG |
| 1174 | rUrArArUrUrUrCrUrArCrUrCrUrUrGrUrArGrArUrGrGrArGrCrArCrGrUrArA |
| | rArUrGrCrGrUrCrCrCrU |
| 1175 | rUrArArUrUrUrCrUrArCrUrCrUrUrGrUrArGrArUrArArCrCrArArUrArUrArC |
| | rArArArGrUrGrCrArArA |
| 1176 | rUrArArUrUrUrCrUrArCrUrCrUrUrGrUrArGrArUrCrArCrUrUrUrGrUrArUrA |
| | rUrUrGrGrUrUrGrArArA |
| 1177 | rUrArArUrUrUrCrUrArCrUrCrUrUrGrUrArGrArUrUrArUrArUrUrGrGrUrUrG |
| | rArArArCrUrGrUrUrArU |
| 1178 | rUrArArUrUrUrCrUrArCrUrCrUrUrGrUrArGrArUrUrUrUrUrUrGrCrArArArC |
| | rCrCrUrGrGrUrUrGrCrU |
| 1179 | rUrArArUrUrUrCrUrArCrUrCrUrUrGrUrArGrArUrCrArArArCrCrCrUrGrGrU |
| | rUrGrCrUrGrUrArUrUrU |
| 1180 | rUrArArUrUrUrCrUrArCrUrCrUrUrGrUrArGrArUrArGrArGrGrCrArArUrArA |
| | rCrCrCrCrCrUrArCrArC |
| 1181 | rUrArArUrUrUrCrUrArCrUrCrUrUrGrUrArGrArUrUrArCrArUrArArArGrArA |
| | rGrArGrGrCrArUrUrUrC |
| 1182 | rUrArArUrUrUrCrUrArCrUrCrUrUrGrUrArGrArUrUrGrUrArCrArArArGrArU |
| | rUrArUrUrUrGrCrArCrG |
| 1183 | rUrArArUrUrUrCrUrArCrUrCrUrUrGrUrArGrArUrCrArCrGrArArCrUrGrGrA |
| | rCrUrGrUrGrUrGrCrArA |
| 1184 | rUrArArUrUrUrCrUrArCrUrCrUrUrGrUrArGrArUrGrGrArGrArArUrGrArUrG |
| | rUrCrCrCrCrGrUrUrGrU |
| 1185 | rUrArArUrUrUrCrUrArCrUrCrUrUrGrUrArGrArUrArArCrCrArCrUrGrUrArU |
| | rArGrArArGrGrUrUrUrU |
| 1186 | rUrArArUrUrUrCrUrArCrUrCrUrUrGrUrArGrArUrUrArGrCrCrUrGrArArUrG |
| | rUrCrUrUrArCrUrGrUrG |
| 1187 | rUrArArUrUrUrCrUrArCrUrCrUrUrGrUrArGrArUrArUrUrGrArUrCrArCrArG |
| | rUrArArGrArCrArUrUrC |
| 1188 | rUrArArUrUrUrCrUrArCrUrCrUrUrGrUrArGrArUrArGrArArArUrUrUrArArU |
| | rUrGrArUrCrArCrArGrU |
| 1189 | rUrArArUrUrUrCrUrArCrUrCrUrUrGrUrArGrArUrGrArCrArArArUrUrGrGrU |
| | rUrCrArUrUrUrArArGrA |
| 1190 | rUrArArUrUrUrCrUrArCrUrCrUrUrGrUrArGrArUrUrUrArArArUrGrArArCrC |
| | rArArUrUrUrGrUrCrUrA |
| 1191 | rUrGrCrArCrGrUrUrCrU |
| | rUrArArUrUrUrCrUrArCrUrCrUrUrGrUrArGrArUrUrCrUrArArArCrUrCrGrA |
| 1192 | rUrArArUrUrUrCrUrArCrUrCrUrUrGrUrArGrArUrUrGrGrArUrUrGrGrArCrU |
| | rCrCrCrCrArGrGrArGrA |
| 1193 | rUrArArUrUrUrCrUrArCrUrCrUrUrGrUrArGrArUrUrCrArArGrUrCrCrCrCrU |
| | rGrCrUrCrArUrCrCrArU |
| 1194 | rUrArArUrUrUrCrUrArCrUrCrUrUrGrUrArGrArUrUrCrGrArGrUrGrUrArUrG |
| | rArArUrGrGrArUrGrArG |
| 1195 | rUrArArUrUrUrCrUrArCrUrCrUrUrGrUrArGrArUrUrUrArUrArUrCrCrCrCrU |
| | rGrUrArGrUrGrUrCrCrC |
| 1196 | rUrArArUrUrUrCrUrArCrUrCrUrUrGrUrArGrArUrArUrUrUrCrUrUrArUrArU |
| | rCrCrCrCrUrGrUrArGrU |
| 1197 | rUrArArUrUrUrCrUrArCrUrCrUrUrGrUrArGrArUrUrUrCrArGrGrGrGrGrArG |
| | rUrUrUrCrUrCrUrArArU |
| 1198 | rUrArArUrUrUrCrUrArCrUrCrUrUrGrUrArGrArUrUrGrGrUrGrCrArGrUrCrA |
| | rCrCrUrGrCrCrArGrCrA |
| 1199 | rUrArArUrUrUrCrUrArCrUrCrUrUrGrUrArGrArUrUrGrGrUrGrGrUrGrGrCrG |
| | rCrUrUrUrCrUrGrCrUrG |
| 1200 | rUrArArUrUrUrCrUrArCrUrCrUrUrGrUrArGrArUrCrCrUrCrCrUrArUrGrArG |
| | rUrUrUrCrUrGrGrCrUrC |
| 1201 | rUrArArUrUrUrCrUrArCrUrCrUrUrGrUrArGrArUrUrGrGrArCrCrUrArArCrC |
| | rArCrCrCrCrArGrUrGrU |
| 1333 | rUrArArUrUrUrCrUrArCrUrCrUrUrGrUrArGrArUrUrUrGrUrCrUrGrGrGrGrA |
| | rCrArGrArArArArGrGrC |
| 1334 | rUrArArUrUrUrCrUrArCrUrCrUrUrGrUrArGrArUrGrCrUrUrCrCrArArGrGrA |
| | rArArUrGrArArGrArUrG |
| 1335 | rUrArArUrUrUrCrUrArCrUrCrUrUrGrUrArGrArUrArArUrGrGrGrCrUrGrArU |
| | rCrUrCrArGrGrCrCrUrG |
| 1336 | rUrArArUrUrUrCrUrArCrUrCrUrUrGrUrArGrArUrArArArGrCrArGrUrUrCrC |
| | rCrArCrCrCrArCrUrCrA |
| 1337 | rUrArArUrUrUrCrUrArCrUrCrUrUrGrUrArGrArUrArGrUrGrGrGrUrGrGrGrA |
| | rArCrUrGrCrUrUrUrGrA |
| 1457 | /5InvdT/ATGTGTTTTTGTCAAAAGACCTTTT/i2FU/rArArU/i2FU//i2FU/rCr |
| | U/i2FA//i2FC/rU/i2FC//12FU//i2FU/rG/12FU//12FA//12FG//i2FA/rUrUrCrUrUrA |
| | rUrCrCrUrUrCrArCrGrArGrGrArArArG/3InvdT/ |
| 1458 | /5InvdT/ATGTGTTTTTGTCAAAAGACCTTTT/12FU/rArArU/i2FU//i2FU/rCr |
| | U/i2FA//i2FC/rU/i2FC//i2FU//12FU/rG/i2FU//12FA//12FG//12FA/rUrGrGrArGr |
| | ArArUrGrArUrGrUrCrCrCrCrGrUrUrGrU/3InvdT/ |

All bases are in upper case Lowercase “r” represents RNA, 2′-hydroxy; bases not modified
by an “r” are DNA
All bases are linked via standard phosphodiester bonds

TABLE 10
LDLR Target Sequences for AsCas12a RR

| SEQ ID NO | Target Sequence (DNA) |
|---|---|
| 665 | GGCAGATGTTCACGCCACGTC |
| 666 | TGAGACCTCGCCGGCCTTGTT |
| 667 | TCTGGGAGGCAGAACAGGCTT |
| 668 | AAGCCTGTTCTGCCTCCCAGA |
| 669 | CGAGGAAAGGAAGAAACCAAA |
| 670 | TTTCCTCGTGAAGGATAAGAG |
| 671 | CCCGGGCCTGTTTCTCTTATC |
| 672 | GGAAGTTTTGAGTTTCTCTCC |
| 673 | CCGTGACACAATCCTCAAACA |
| 674 | TGTTTGAGGATTGTGTCACGG |
| 675 | ATGTTTGAGGATTGTGTCACG |
| 676 | TCTTCCATGTTTGAGGATTGT |
| 677 | CTTTCATCTTCCATGTTTGAG |
| 678 | ACACACAACAGCAGATGGCAC |
| 679 | GTTGGTGCCATCTGCTGTTGT |
| 680 | TGGCTTAAGGAAGGTACAAAC |
| 681 | TTAAGCCAGGAAAGGGATTCA |
| 682 | ACGCCATGAATCCCTTTCCTG |
| 683 | GACGCCATGAATCCCTTTCCT |
| 684 | TGGCGTCGGAAATGATCTGGC |
| 685 | GCCAGATCATTTCCGACGCCA |
| 686 | TGGTGGCACCGAGACCAAACT |
| 687 | CCAAATGATGCCACTTCCCAG |
| 688 | ATTCCCGTGGTCTCCTTGCAC |
| 689 | TTCCCGTGGTCTCCTTGCACT |
| 690 | CGTGGTCTCCTTGCACTTTCT |
| 691 | GAGTTGTACACTGTGTACATT |
| 692 | ATCCCAACACACACGACAGAA |
| 693 | TGGCCTGGGATCCCATCCCAA |
| 694 | AGGCCAGGGAAAGCCCGTGTC |
| 695 | TTGACACGGGCTTTCCCTGGC |
| 696 | CGGCATTCATTGACACGGGCT |
| 697 | GGTCAACCTGCCCCTCTCTGT |
| 698 | CGATCACGGCTTTGAAGTCCC |
| 699 | AAGCCGTGATCGTGAATATCG |
| 700 | CCCACCTAGTGCTTCCACTTC |
| 701 | ACTTCTATGCAAATGCCTCCA |
| 702 | CTTCTATGCAAATGCCTCCAA |
| 703 | CTTCCCCAATCTTGTCGTTGA |
| 704 | GTCTGGATCGTTTGACGGGAC |
| 705 | GAAAGAACCTGAAGTCCCGTC |
| 706 | GGTTCTTTCTGAAATCGCCGT |
| 707 | GAGAGACAGTGACAGCCTCCG |
| 708 | CGCGGGAGTCTGACGGAGGCT |
| 709 | TCAGACTCCCGCGTGAAGATG |
| 710 | TTGTGACATCTTCACGCGGGA |
| 711 | GCGTGAAGATGTCACAAGGGA |
| 712 | TGGGGACAATTGCCAATCCCT |
| 713 | CAGGGACAAAACACTGTGTCC |
| 714 | TGCACTGGGGGGGACACAGTG |
| 715 | CTGCACTGGGGGGGACACAGT |
| 716 | CCCCAGTGCAGGGAACCGTGA |
| 717 | AAACCAGAAAGGCTTATCACG |
| 718 | GAGCACGTAAATGCGTCCCTG |
| 719 | CACTATCTGTACAGGGACGCA |
| 720 | TGTACAGATAGTGGGGATTTT |
| 721 | GTGACTATTCTCGGGGCCCTG |
| 722 | GAGGCAATAACCCCCTACACA |

TABLE 11
LDLR Targeting Sequences for AsCas12a RR

| SEQ | |
|---|---|
| ID NO | gDNA targeting Domain (RNA) |
| 751 | rGrGrCrArGrArUrGrUrUrCrArCrGrCrCrArCrGrUrC |
| 752 | rUrGrArGrArCrCrUrCrGrCrCrGrGrCrCrUrUrGrUrU |
| 753 | rUrCrUrGrGrGrArGrGrCrArGrArArCrArGrGrCrUrU |
| 754 | rArArGrCrCrUrGrUrUrCrUrGrCrCrUrCrCrCrArGrA |
| 755 | rCrGrArGrGrArArArGrGrArArGrArArArCrCrArArA |
| 756 | rUrUrUrCrCrUrCrGrUrGrArArGrGrArUrArArGrArG |
| 757 | rCrCrCrGrGrGrCrCrUrGrUrUrUrCrUrCrUrUrArUrC |
| 758 | rGrGrArArGrUrUrUrUrGrArGrUrUrUrCrUrCrUrCrC |
| 759 | rCrCrGrUrGrArCrArCrArArUrCrCrUrCrArArArCrA |
| 760 | rUrGrUrUrUrGrArGrGrArUrUrGrUrGrUrCrArCrGrG |
| 761 | rArUrGrUrUrUrGrArGrGrArUrUrGrUrGrUrCrArCrG |
| 762 | rUrCrUrUrCrCrArUrGrUrUrUrGrArGrGrArUrUrGrU |
| 763 | rCrUrUrUrCrArUrCrUrUrCrCrArUrGrUrUrUrGrArG |
| 764 | rArCrArCrArCrArArCrArGrCrArGrArUrGrGrCrArC |
| 765 | rGrUrUrGrGrUrGrCrCrArUrCrUrGrCrUrGrUrUrGrU |
| 766 | rUrGrGrCrUrUrArArGrGrArArGrGrUrArCrArArArC |
| 767 | rUrUrArArGrCrCrArGrGrArArArGrGrGrArUrUrCrA |
| 768 | rArCrGrCrCrArUrGrArArUrCrCrCrUrUrUrCrCrUrG |
| 769 | rGrArCrGrCrCrArUrGrArArUrCrCrCrUrUrUrCrCrU |
| 770 | rUrGrGrCrGrUrCrGrGrArArArUrGrArUrCrUrGrGrC |
| 771 | rGrCrCrArGrArUrCrArUrUrUrCrCrGrArCrGrCrCrA |
| 772 | rUrGrGrUrGrGrCrArCrCrGrArGrArCrCrArArArCrU |
| 773 | rCrCrArArArUrGrArUrGrCrCrArCrUrUrCrCrCrArG |
| 774 | rArUrUrCrCrCrGrUrGrGrUrCrUrCrCrUrUrGrCrArC |
| 775 | rUrUrCrCrCrGrUrGrGrUrCrUrCrCrUrUrGrCrArCrU |
| 776 | rCrGrUrGrGrUrCrUrCrCrUrUrGrCrArCrUrUrUrCrU |
| 777 | rGrArGrUrUrGrUrArCrArCrUrGrUrGrUrArCrArUrU |
| 778 | rArUrCrCrCrArArCrArCrArCrArCrGrArCrArGrArA |
| 779 | rUrGrGrCrCrUrGrGrGrArUrCrCrCrArUrCrCrCrArA |
| 780 | rArGrGrCrCrArGrGrGrArArArGrCrCrCrGrUrGrUrC |
| 781 | rUrUrGrArCrArCrGrGrGrCrUrUrUrCrCrCrUrGrGrC |
| 782 | rCrGrGrCrArUrUrCrArUrUrGrArCrArCrGrGrGrCrU |
| 783 | rGrGrUrCrArArCrCrUrGrCrCrCrCrUrCrUrCrUrGrU |
| 784 | rCrGrArUrCrArCrGrGrCrUrUrUrGrArArGrUrCrCrC |
| 785 | rArArGrCrCrGrUrGrArUrCrGrUrGrArArUrArUrCrG |
| 786 | rCrCrCrArCrCrUrArGrUrGrCrUrUrCrCrArCrUrUrC |
| 787 | rArCrUrUrCrUrArUrGrCrArArArUrGrCrCrUrCrCrA |
| 788 | rCrUrUrCrUrArUrGrCrArArArUrGrCrCrUrCrCrArA |
| 789 | rCrUrUrCrCrCrCrArArUrCrUrUrGrUrCrGrUrUrGrA |
| 790 | rGrUrCrUrGrGrArUrCrGrUrUrUrGrArCrGrGrGrArC |
| 791 | rGrArArArGrArArCrCrUrGrArArGrUrCrCrCrGrUrC |
| 792 | rGrGrUrUrCrUrUrUrCrUrGrArArArUrCrGrCrCrGrU |
| 793 | rGrArGrArGrArCrArGrUrGrArCrArGrCrCrUrCrCrG |
| 794 | rCrGrCrGrGrGrArGrUrCrUrGrArCrGrGrArGrGrCrU |
| 795 | rUrCrArGrArCrUrCrCrCrGrCrGrUrGrArArGrArUrG |
| 796 | rUrUrGrUrGrArCrArUrCrUrUrCrArCrGrCrGrGrGrA |
| 797 | rGrCrGrUrGrArArGrArUrGrUrCrArCrArArGrGrGrA |
| 798 | rUrGrGrGrGrArCrArArUrUrGrCrCrArArUrCrCrCrU |
| 799 | rCrArGrGrGrArCrArArArArCrArCrUrGrUrGrUrCrC |
| 800 | rUrGrCrArCrUrGrGrGrGrGrGrGrArCrArCrArGrUrG |
| 801 | rCrUrGrCrArCrUrGrGrGrGrGrGrGrArCrArCrArGrU |
| 802 | rCrCrCrCrArGrUrGrCrArGrGrGrArArCrCrGrUrGrA |
| 803 | rArArArCrCrArGrArArArGrGrCrUrUrArUrCrArCrG |
| 804 | rGrArGrCrArCrGrUrArArArUrGrCrGrUrCrCrCrUrG |
| 805 | rCrArCrUrArUrCrUrGrUrArCrArGrGrGrArCrGrCrA |
| 806 | rUrGrUrArCrArGrArUrArGrUrGrGrGrGrArUrUrUrU |
| 807 | rGrUrGrArCrUrArUrUrCrUrCrGrGrGrGrCrCrCrUrG |
| 808 | rGrArGrGrCrArArUrArArCrCrCrCrCrUrArCrArCrA |
| 809 | rGrUrUrCrGrUrGrCrArArArUrArArUrCrUrUrUrGrU |
| 810 | rArArArArArGrCrGrUrUrGrCrArCrArCrArGrUrCrC |
| 811 | rCrGrUrUrGrUrArUrGrUrArUrGrArGrUrGrGrCrUrU |
| 812 | rGrGrCrUrArCrArArArArArCrCrUrUrCrUrArUrArC |
| 813 | rUrUrUrArArGrArArArUrUrUrArArUrUrGrArUrCrA |
| 814 | rArGrArArCrUrCrCrGrGrGrCrArArGrArUrGrCrArA |
| 815 | rGrGrCrArArGrArUrGrCrArArUrCrUrCrCrGrUrGrG |
| 816 | rArCrUrCrArGrArGrArArGrCrGrArCrArGrCrArGrA |
| 817 | rCrUrGrCrUrCrArUrCrCrArUrUrCrArUrArCrArCrU |
| 818 | rUrUrCrArUrArCrArCrUrCrGrArCrArArArUrArUrC |
| 819 | rUrArCrArCrUrCrGrArCrArArArUrArUrCrUrGrUrU |
| 820 | rArCrArArGrCrCrUrArCrGrGrCrArGrArGrCrArUrG |
| 821 | rCrUrGrUrArGrUrGrUrCrCrCrArCrArArGrCrCrUrA |
| 822 | rGrGrGrGrGrArGrUrUrUrCrUrCrUrArArUrGrArArG |
| 823 | rUrUrArGrArGrArArArCrUrCrCrCrCrCrUrGrArArG |
| 824 | rUrGrCrCrArGrUrUrGrCrUrUrUrUrCrUrGrUrCrCrC |
| 825 | rArGrArUrUrArArArCrArGrGrGrGrGrArCrGrGrUrG |
| 826 | rUrCrUrCrCrUrGrGrUrUrUrUrGrUrGrGrUrGrGrUrG |
| 827 | rArGrGrCrUrCrCrArGrArArArCrCrArCrArUrCrUrU |
| 828 | rCrUrCrCrUrArUrGrArGrUrUrUrCrUrGrGrCrUrCrU |
| 829 | rCrGrCrCrUrCrGrCrUrCrUrGrCrUrGrGrGrGrArCrC |
| 830 | rCrCrGrCrCrUrCrGrCrUrCrUrGrCrUrGrGrGrGrArC |
| 831 | rCrCrGrCrUrCrCrArGrGrCrUrCrUrArUrCrUrCrCrC |
| 832 | rGrGrCrUrCrUrArUrCrUrCrCrCrArCrCrArGrUrGrC |
| 833 | rUrGrUrUrGrGrGrCrUrCrArGrCrCrCrGrUrGrGrGrA |
| 834 | rArCrGrGrGrCrUrGrArGrCrCrCrArArCrArGrGrGrA |
| 835 | rUrCrArGrCrCrUrGrUrCrCrCrUrGrUrUrGrGrGrCrU |
| 836 | rArUrCrCrArCrCrCrArGrUrArArGrGrCrArCrCrArU |

All bases are in upper case Lowercase “r” represents RNA,
2′-hydroxy; bases not modified by an “r” are DNA
All bases are linked via standard phosphodiester bonds

TABLE 12
gRNA sequences for targeting LDLR with AsCas12a (RR)

| SEQ ID NO | gRNA sequence |
|---|---|
| 1202 | rUrArArUrUrUrCrUrArCrUrCrUrUrGrUrArGrArUrGrGrCrArGrArUrGrUrUrCr |
| | ArCrGrCrCrArCrGrUrC |
| 1203 | rUrArArUrUrUrCrUrArCrUrCrUrUrGrUrArGrArUrUrGrArGrArCrCrUrCrGrCr |
| | CrGrGrCrCrUrUrGrUrU |
| 1204 | rUrArArUrUrUrCrUrArCrUrCrUrUrGrUrArGrArUrUrCrUrGrGrGrArGrGrCrAr |
| | GrArArCrArGrGrCrUrU |
| 1205 | rUrArArUrUrUrCrUrArCrUrCrUrUrGrUrArGrArUrArArGrCrCrUrGrUrUrCrUr |
| | GrCrCrUrCrCrCrArGrA |
| 1206 | rUrArArUrUrUrCrUrArCrUrCrUrUrGrUrArGrArUrCrGrArGrGrArArArGrGrAr |
| | ArGrArArArCrCrArArA |
| 1207 | rUrArArUrUrUrCrUrArCrUrCrUrUrGrUrArGrArUrUrUrUrCrCrUrCrGrUrGrAr |
| | ArGrGrArUrArArGrArG |
| 1208 | rUrArArUrUrUrCrUrArCrUrCrUrUrGrUrArGrArUrCrCrCrGrGrGrCrCrUrGrUr |
| | UrUrCrUrCrUrUrArUrC |
| 1209 | rUrArArUrUrUrCrUrArCrUrCrUrUrGrUrArGrArUrGrGrArArGrUrUrUrUrGrAr |
| | GrUrUrUrCrUrCrUrCrC |
| 1210 | rUrArArUrUrUrCrUrArCrUrCrUrUrGrUrArGrArUrCrCrGrUrGrArCrArCrArAr |
| | UrCrCrUrCrArArArCrA |
| 1211 | rUrArArUrUrUrCrUrArCrUrCrUrUrGrUrArGrArUrUrGrUrUrUrGrArGrGrArUr |
| | UrGrUrGrUrCrArCrGrG |
| 1212 | rUrArArUrUrUrCrUrArCrUrCrUrUrGrUrArGrArUrArUrGrUrUrUrGrArGrGrAr |
| | UrUrGrUrGrUrCrArCrG |
| 1213 | rUrArArUrUrUrCrUrArCrUrCrUrUrGrUrArGrArUrUrCrUrUrCrCrArUrGrUrUr |
| | UrGrArGrGrArUrUrGrU |
| 1214 | rUrArArUrUrUrCrUrArCrUrCrUrUrGrUrArGrArUrCrUrUrUrCrArUrCrUrUrCr |
| | CrArUrGrUrUrUrGrArG |
| 1215 | rUrArArUrUrUrCrUrArCrUrCrUrUrGrUrArGrArUrArCrArCrArCrArArCrArGr |
| | CrArGrArUrGrGrCrArC |
| 1216 | rUrArArUrUrUrCrUrArCrUrCrUrUrGrUrArGrArUrGrUrUrGrGrUrGrCrCrArUr |
| | CrUrGrCrUrGrUrUrGrU |
| 1217 | rUrArArUrUrUrCrUrArCrUrCrUrUrGrUrArGrArUrUrGrGrCrUrUrArArGrGrAr |
| | ArGrGrUrArCrArArArC |
| 1218 | rUrArArUrUrUrCrUrArCrUrCrUrUrGrUrArGrArUrUrUrArArGrCrCrArGrGrAr |
| | ArArGrGrGrArUrUrCrA |
| 1219 | rUrArArUrUrUrCrUrArCrUrCrUrUrGrUrArGrArUrArCrGrCrCrArUrGrArArUr |
| | CrCrCrUrUrUrCrCrUrG |
| 1220 | rUrArArUrUrUrCrUrArCrUrCrUrUrGrUrArGrArUrGrArCrGrCrCrArUrGrArAr |
| | UrCrCrCrUrUrUrCrCrU |
| 1221 | rUrArArUrUrUrCrUrArCrUrCrUrUrGrUrArGrArUrUrGrGrCrGrUrCrGrGrArAr |
| | ArUrGrArUrCrUrGrGrC |
| 1222 | rUrArArUrUrUrCrUrArCrUrCrUrUrGrUrArGrArUrGrCrCrArGrArUrCrArUrUr |
| | UrCrCrGrArCrGrCrCrA |
| 1223 | rUrArArUrUrUrCrUrArCrUrCrUrUrGrUrArGrArUrUrGrGrUrGrGrCrArCrCrGr |
| | ArGrArCrCrArArArCrU |
| 1224 | rUrArArUrUrUrCrUrArCrUrCrUrUrGrUrArGrArUrCrCrArArArUrGrArUrGrCr |
| | CrArCrUrUrCrCrCrArG |
| 1225 | rUrArArUrUrUrCrUrArCrUrCrUrUrGrUrArGrArUrArUrUrCrCrCrGrUrGrGrUr |
| | CrUrCrCrUrUrGrCrArC |
| 1226 | rUrArArUrUrUrCrUrArCrUrCrUrUrGrUrArGrArUrUrUrCrCrCrGrUrGrGrUrCr |
| | UrCrCrUrUrGrCrArCrU |
| 1227 | rUrArArUrUrUrCrUrArCrUrCrUrUrGrUrArGrArUrCrGrUrGrGrUrCrUrCrCrUr |
| | UrGrCrArCrUrUrUrCrU |
| 1228 | rUrArArUrUrUrCrUrArCrUrCrUrUrGrUrArGrArUrGrArGrUrUrGrUrArCrArCr |
| | UrGrUrGrUrArCrArUrU |
| 1229 | rUrArArUrUrUrCrUrArCrUrCrUrUrGrUrArGrArUrArUrCrCrCrArArCrArCrAr |
| | CrArCrGrArCrArGrArA |
| 1230 | rUrArArUrUrUrCrUrArCrUrCrUrUrGrUrArGrArUrUrGrGrCrCrUrGrGrGrArUr |
| | CrCrCrArUrCrCrCrArA |
| 1231 | rUrArArUrUrUrCrUrArCrUrCrUrUrGrUrArGrArUrArGrGrCrCrArGrGrGrArAr |
| | ArGrCrCrCrGrUrGrUrC |
| 1232 | rUrArArUrUrUrCrUrArCrUrCrUrUrGrUrArGrArUrUrUrGrArCrArCrGrGrGrCr |
| | UrUrUrCrCrCrUrGrGrC |
| 1233 | rUrArArUrUrUrCrUrArCrUrCrUrUrGrUrArGrArUrCrGrGrCrArUrUrCrArUrUr |
| | GrArCrArCrGrGrGrCrU |
| 1234 | rUrArArUrUrUrCrUrArCrUrCrUrUrGrUrArGrArUrGrGrUrCrArArCrCrUrGrCr |
| | CrCrCrUrCrUrCrUrGrU |
| 1235 | rUrArArUrUrUrCrUrArCrUrCrUrUrGrUrArGrArUrCrGrArUrCrArCrGrGrCrUr |
| | UrUrGrArArGrUrCrCrC |
| 1236 | rUrArArUrUrUrCrUrArCrUrCrUrUrGrUrArGrArUrArArGrCrCrGrUrGrArUrCr |
| | GrUrGrArArUrArUrCrG |
| 1237 | rUrArArUrUrUrCrUrArCrUrCrUrUrGrUrArGrArUrCrCrCrArCrCrUrArGrUrGr |
| | CrUrUrCrCrArCrUrUrC |
| 1238 | rUrArArUrUrUrCrUrArCrUrCrUrUrGrUrArGrArUrArCrUrUrCrUrArUrGrCrAr |
| | ArArUrGrCrCrUrCrCrA |
| 1239 | rUrArArUrUrUrCrUrArCrUrCrUrUrGrUrArGrArUrCrUrUrCrUrArUrGrCrArAr |
| | ArUrGrCrCrUrCrCrArA |
| 1240 | rUrArArUrUrUrCrUrArCrUrCrUrUrGrUrArGrArUrCrUrUrCrCrCrCrArArUrCr |
| | UrUrGrUrCrGrUrUrGrA |
| 1241 | rUrArArUrUrUrCrUrArCrUrCrUrUrGrUrArGrArUrGrUrCrUrGrGrArUrCrGrUr |
| | UrUrGrArCrGrGrGrArC |
| 1242 | rUrArArUrUrUrCrUrArCrUrCrUrUrGrUrArGrArUrGrArArArGrArArCrCrUrGr |
| | ArArGrUrCrCrCrGrUrC |
| 1243 | rUrArArUrUrUrCrUrArCrUrCrUrUrGrUrArGrArUrGrGrUrUrCrUrUrUrCrUrGr |
| | ArArArUrCrGrCrCrGrU |
| 1244 | rUrArArUrUrUrCrUrArCrUrCrUrUrGrUrArGrArUrGrArGrArGrArCrArGrUrGr |
| | ArCrArGrCrCrUrCrCrG |
| 1245 | rUrArArUrUrUrCrUrArCrUrCrUrUrGrUrArGrArUrCrGrCrGrGrGrArGrUrCrUr |
| | GrArCrGrGrArGrGrCrU |
| 1246 | rUrArArUrUrUrCrUrArCrUrCrUrUrGrUrArGrArUrUrCrArGrArCrUrCrCrCrGr |
| | CrGrUrGrArArGrArUrG |
| 1247 | rUrArArUrUrUrCrUrArCrUrCrUrUrGrUrArGrArUrUrUrGrUrGrArCrArUrCrUr |
| | UrCrArCrGrCrGrGrGrA |
| 1248 | rUrArArUrUrUrCrUrArCrUrCrUrUrGrUrArGrArUrGrCrGrUrGrArArGrArUrGr |
| | UrCrArCrArArGrGrGrA |
| 1249 | rUrArArUrUrUrCrUrArCrUrCrUrUrGrUrArGrArUrUrGrGrGrGrArCrArArUrUr |
| | GrCrCrArArUrCrCrCrU |
| 1250 | rUrArArUrUrUrCrUrArCrUrCrUrUrGrUrArGrArUrCrArGrGrGrArCrArArArAr |
| | CrArCrUrGrUrGrUrCrC |
| 1251 | rUrArArUrUrUrCrUrArCrUrCrUrUrGrUrArGrArUrUrGrCrArCrUrGrGrGrGrGr |
| | GrGrArCrArCrArGrUrG |
| 1252 | rUrArArUrUrUrCrUrArCrUrCrUrUrGrUrArGrArUrCrUrGrCrArCrUrGrGrGrGr |
| | GrGrGrArCrArCrArGrU |
| 1253 | rUrArArUrUrUrCrUrArCrUrCrUrUrGrUrArGrArUrCrCrCrCrArGrUrGrCrArGr |
| | GrGrArArCrCrGrUrGrA |
| 1254 | rUrArArUrUrUrCrUrArCrUrCrUrUrGrUrArGrArUrArArArCrCrArGrArArArGr |
| | GrCrUrUrArUrCrArCrG |
| 1255 | rUrArArUrUrUrCrUrArCrUrCrUrUrGrUrArGrArUrGrArGrCrArCrGrUrArArAr |
| | UrGrCrGrUrCrCrCrUrG |
| 1256 | rUrArArUrUrUrCrUrArCrUrCrUrUrGrUrArGrArUrCrArCrUrArUrCrUrGrUrAr |
| | CrArGrGrGrArCrGrCrA |
| 1257 | rUrArArUrUrUrCrUrArCrUrCrUrUrGrUrArGrArUrUrGrUrArCrArGrArUrArGr |
| | UrGrGrGrGrArUrUrUrU |
| 1258 | rUrArArUrUrUrCrUrArCrUrCrUrUrGrUrArGrArUrGrUrGrArCrUrArUrUrCrUr |
| | CrGrGrGrGrCrCrCrUrG |
| 1259 | rUrArArUrUrUrCrUrArCrUrCrUrUrGrUrArGrArUrGrArGrGrCrArArUrArArCr |
| | CrCrCrCrUrArCrArCrA |
| 1260 | rUrArArUrUrUrCrUrArCrUrCrUrUrGrUrArGrArUrGrUrUrCrGrUrGrCrArArAr |
| | UrArArUrCrUrUrUrGrU |
| 1261 | rUrArArUrUrUrCrUrArCrUrCrUrUrGrUrArGrArUrArArArArArGrCrGrUrUrGr |
| | CrArCrArCrArGrUrCrC |
| 1262 | rUrArArUrUrUrCrUrArCrUrCrUrUrGrUrArGrArUrCrGrUrUrGrUrArUrGrUrAr |
| | UrGrArGrUrGrGrCrUrU |
| 1263 | rUrArArUrUrUrCrUrArCrUrCrUrUrGrUrArGrArUrGrGrCrUrArCrArArArArAr |
| | CrCrUrUrCrUrArUrArC |
| 1264 | rUrArArUrUrUrCrUrArCrUrCrUrUrGrUrArGrArUrUrUrUrArArGrArArArUrUr |
| | UrArArUrUrGrArUrCrA |
| 1265 | rUrArArUrUrUrCrUrArCrUrCrUrUrGrUrArGrArUrArGrArArCrUrCrCrGrGrGr |
| | CrArArGrArUrGrCrArA |
| 1266 | rUrArArUrUrUrCrUrArCrUrCrUrUrGrUrArGrArUrGrGrCrArArGrArUrGrCrAr |
| | ArUrCrUrCrCrGrUrGrG |
| 1267 | rUrArArUrUrUrCrUrArCrUrCrUrUrGrUrArGrArUrArCrUrCrArGrArGrArArGr |
| | CrGrArCrArGrCrArGrA |
| 1268 | rUrArArUrUrUrCrUrArCrUrCrUrUrGrUrArGrArUrCrUrGrCrUrCrArUrCrCrAr |
| | UrUrCrArUrArCrArCrU |
| 1269 | rUrArArUrUrUrCrUrArCrUrCrUrUrGrUrArGrArUrUrUrCrArUrArCrArCrUrCr |
| | GrArCrArArArUrArUrC |
| 1270 | rUrArArUrUrUrCrUrArCrUrCrUrUrGrUrArGrArUrUrArCrArCrUrCrGrArCrAr |
| | ArArUrArUrCrUrGrUrU |
| 1271 | rUrArArUrUrUrCrUrArCrUrCrUrUrGrUrArGrArUrArCrArArGrCrCrUrArCrGr |
| | GrCrArGrArGrCrArUrG |
| 1272 | rUrArArUrUrUrCrUrArCrUrCrUrUrGrUrArGrArUrCrUrGrUrArGrUrGrUrCrCr |
| | CrArCrArArGrCrCrUrA |
| 1273 | rUrArArUrUrUrCrUrArCrUrCrUrUrGrUrArGrArUrGrGrGrGrGrArGrUrUrUrCr |
| | UrCrUrArArUrGrArArG |
| 1274 | rUrArArUrUrUrCrUrArCrUrCrUrUrGrUrArGrArUrUrUrArGrArGrArArArCrUr |
| | CrCrCrCrCrUrGrArArG |
| 1275 | rUrArArUrUrUrCrUrArCrUrCrUrUrGrUrArGrArUrUrGrCrCrArGrUrUrGrCrUr |
| | UrUrUrCrUrGrUrCrCrC |
| 1276 | rUrArArUrUrUrCrUrArCrUrCrUrUrGrUrArGrArUrArGrArUrUrArArArCrArGr |
| | GrGrGrGrArCrGrGrUrG |
| 1277 | rUrArArUrUrUrCrUrArCrUrCrUrUrGrUrArGrArUrUrCrUrCrCrUrGrGrUrUrUr |
| | UrGrUrGrGrUrGrGrUrG |
| 1278 | rUrArArUrUrUrCrUrArCrUrCrUrUrGrUrArGrArUrArGrGrCrUrCrCrArGrArAr |
| | ArCrCrArCrArUrCrUrU |
| 1279 | rUrArArUrUrUrCrUrArCrUrCrUrUrGrUrArGrArUrCrUrCrCrUrArUrGrArGrUr |
| | UrUrCrUrGrGrCrUrCrU |
| 1280 | rUrArArUrUrUrCrUrArCrUrCrUrUrGrUrArGrArUrCrGrCrCrUrCrGrCrUrCrUr |
| | GrCrUrGrGrGrGrArCrC |
| 1281 | rUrArArUrUrUrCrUrArCrUrCrUrUrGrUrArGrArUrCrCrGrCrCrUrCrGrCrUrCrU |
| | rGrCrUrGrGrGrGrArC |
| 1282 | rUrArArUrUrUrCrUrArCrUrCrUrUrGrUrArGrArUrCrCrGrCrUrCrCrArGrGrCr |
| | UrCrUrArUrCrUrCrCrC |
| 1283 | rUrArArUrUrUrCrUrArCrUrCrUrUrGrUrArGrArUrGrGrCrUrCrUrArUrCrUrCr |
| | CrCrArCrCrArGrUrGrC |
| 1284 | rUrArArUrUrCrUrArCrUrCrUrUrGrUrArGrArUrUrGrUrUrGrGrGrCrUrCrAr |
| | GrCrCrCrGrUrGrGrGrA |
| 1285 | rUrArArUrUrUrCrUrArCrUrCrUrUrGrUrArGrArUrArCrGrGrGrCrUrGrArGrCr |
| | CrCrArArCrArGrGrGrA |
| 1286 | rUrArArUrUrUrCrUrArCrUrCrUrUrGrUrArGrArUrUrCrArGrCrCrUrGrUrCrCr |
| | CrUrGrUrUrGrGrGrCrU |
| 1287 | rUrArArUrUrUrCrUrArCrUrCrUrUrGrUrArGrArUrArUrCrCrArCrCrCrArGrUr |
| | ArArGrGrCrArCrCrArU |

All bases are in upper case Lowercase “r” represents RNA, 2′-hydroxy; bases not modified
by an “r” are DNA
All bases are linked via standard phosphodiester bonds

TABLE 13
LDLR Target Sequences for AsCas12a RVR

| SEQ ID NO | Target Sequence (DNA) |
|---|---|
| 837 | TAAAACAAAGCTCTGGCAGGC |
| 838 | CAAGCCTTGCTAGACAGCCAG |
| 839 | GAGAACTGCCATTGTCGTCTT |
| 840 | TCCGCCCACCTAGTGCTTCCA |
| 841 | CAAATGCCTCCAAGCCATTCA |
| 842 | TGTTTAAAACATGCACGGTGA |
| 843 | GTTGTATTTCTGAAATAGGGT |
| 844 | ACGGTTCCCTGCACTGGGGGG |
| 845 | TGTACAGGGACGCATTTACGT |
| 846 | TTTGCACTTTGTATATTGGTT |
| 847 | TTGGTTGAAACTGTTATCACT |
| 848 | AGTGATAACAGTTTCAACCAA |
| 849 | TAAGTGATAACAGTTTCAACC |
| 850 | TATAAGTGATAACAGTTTCAA |
| 851 | TATATAAGTGATAACAGTTTC |
| 852 | TATATATAAGTGATAACAGTT |
| 853 | TACAAAGATTATTTGCACGAA |
| 854 | TATGAGTGGCTTCTGGGAGAT |
| 855 | AGTGGCTTCTGGGAGATGGGT |
| 856 | CAGTGGTTTAAAAAGTGACAC |
| 857 | GAAGGTTTTTGTAGCCTGAAT |
| 858 | AATGGATGAGCAGGGGACTTG |
| 859 | TGTTGAGTGCTAAGTGCGAAC |
| 860 | CCCTGTAGTGTCCCACAAGCC |
| 861 | TCCCCTGTAGTGTCCCACAAG |
| 862 | GGGACAGAAAAGCAACTGGCA |
| 863 | GTGCAGTCACCTGCCAGCATA |
| 864 | CTGGCAGGTGACTGCACCATA |
| 865 | AGTTTCTGGCTCTCACCAGCT |

TABLE 14
LDLR Targeting Sequences for AsCas12a RVR

| SEQ | |
|---|---|
| ID NO | gDNA targeting Domain (RNA) |
| 866 | rUrArArArArCrArArArGrCrUrCrUrGrGrCrArGrGrC |
| 867 | rCrArArGrCrCrUrUrGrCrUrArGrArCrArGrCrCrArG |
| 868 | rGrArGrArArCrUrGrCrCrArUrUrGrUrCrGrUrCrUrU |
| 869 | rUrCrCrGrCrCrCrArCrCrUrArGrUrGrCrUrUrCrCrA |
| 870 | rCrArArArUrGrCrCrUrCrCrArArGrCrCrArUrUrCrA |
| 871 | rUrGrUrUrUrArArArArCrArUrGrCrArCrGrGrUrGrA |
| 872 | rGrUrUrGrUrArUrUrUrCrUrGrArArArUrArGrGrGrU |
| 873 | rArCrGrGrUrUrCrCrCrUrGrCrArCrUrGrGrGrGrGrG |
| 874 | rUrGrUrArCrArGrGrGrArCrGrCrArUrUrUrArCrGrU |
| 875 | rUrUrUrGrCrArCrUrUrUrGrUrArUrArUrUrGrGrUrU |
| 876 | rUrUrGrGrUrUrGrArArArCrUrGrUrUrArUrCrArCrU |
| 877 | rArGrUrGrArUrArArCrArGrUrUrUrCrArArCrCrArA |
| 878 | rUrArArGrUrGrArUrArArCrArGrUrUrUrCrArArCrC |
| 879 | rUrArUrArArGrUrGrArUrArArCrArGrUrUrUrCrArA |
| 880 | rUrArUrArUrArArGrUrGrArUrArArCrArGrUrUrUrC |
| 881 | rUrArUrArUrArUrArArGrUrGrArUrArArCrArGrUrU |
| 882 | rUrArCrArArArGrArUrUrArUrUrUrGrCrArCrGrArA |
| 883 | rUrArUrGrArGrUrGrGrCrUrUrCrUrGrGrGrArGrArU |
| 884 | rArGrUrGrGrCrUrUrCrUrGrGrGrArGrArUrGrGrGrU |
| 885 | rCrArGrUrGrGrUrUrUrArArArArArGrUrGrArCrArC |
| 886 | rGrArArGrGrUrUrUrUrUrGrUrArGrCrCrUrGrArArU |
| 887 | rArArUrGrGrArUrGrArGrCrArGrGrGrGrArCrUrUrG |
| 888 | rUrGrUrUrGrArGrUrGrCrUrArArGrUrGrCrGrArArC |
| 889 | rCrCrCrUrGrUrArGrUrGrUrCrCrCrArCrArArGrCrC |
| 890 | rUrCrCrCrCrUrGrUrArGrUrGrUrCrCrCrArCrArArG |
| 891 | rGrGrGrArCrArGrArArArArGrCrArArCrUrGrGrCrA |
| 892 | rGrUrGrCrArGrUrCrArCrCrUrGrCrCrArGrCrArUrA |
| 893 | rCrUrGrGrCrArGrGrUrGrArCrUrGrCrArCrCrArUrA |
| 894 | rArGrUrUrUrCrUrGrGrCrUrCrUrCrArCrCrArGrCrU |

All bases are in upper case Lowercase “r” represents RNA,
2′-hydroxy; bases not modified by an “r” are DNA
All bases are linked via standard phosphodiester bonds

TABLE 15
gRNA sequences for targeting LDLR with AsCas12a (RVR)

| SEQ ID NO | gRNA Sequence |
|---|---|
| 1288 | rUrArArUrUrUrCrUrArCrUrCrUrUrGrUrArGrArUrUrArArArArCrArArArGrCr |
| | UrCrUrGrGrCrArGrGrC |
| 1289 | rUrArArUrUrUrCrUrArCrUrCrUrUrGrUrArGrArUrCrArArGrCrCrUrUrGrCrUr |
| | ArGrArCrArGrCrCrArG |
| 1290 | UrUrGrUrCrGrUrCrUrU |
| | rUrArArUrUrUrCrUrArCrUrCrUrUrGrUrArGrArUrGrArGrArArCrUrGrCrCrAr |
| 1291 | rUrArArUrUrUrCrUrArCrUrCrUrUrGrUrArGrArUrUrCrCrGrCrCrCrArCrCrUr |
| | ArGrUrGrCrUrUrCrCrA |
| 1292 | rUrArArUrUrUrCrUrArCrUrCrUrUrGrUrArGrArUrCrArArArUrGrCrCrUrCrCr |
| | ArArGrCrCrArUrUrCrA |
| 1293 | rUrArArUrUrUrCrUrArCrUrCrUrUrGrUrArGrArUrUrGrUrUrUrArArArArCrAr |
| | UrGrCrArCrGrGrUrGrA |
| 1294 | rUrArArUrUrUrCrUrArCrUrCrUrUrGrUrArGrArUrGrUrUrGrUrArUrUrUrCrUr |
| | GrArArArUrArGrGrGrU |
| 1295 | rUrArArUrUrUrCrUrArCrUrCrUrUrGrUrArGrArUrArCrGrGrUrUrCrCrCrUrGr |
| | CrArCrUrGrGrGrGrGrG |
| 1296 | rUrArArUrUrUrCrUrArCrUrCrUrUrGrUrArGrArUrUrGrUrArCrArGrGrGrArCr |
| | GrCrArUrUrUrArCrGrU |
| 1297 | rUrArArUrUrUrCrUrArCrUrCrUrUrGrUrArGrArUrUrUrUrGrCrArCrUrUrUrGr |
| | UrArUrArUrUrGrGrUrU |
| 1298 | rUrArArUrUrUrCrUrArCrUrCrUrUrGrUrArGrArUrUrUrGrGrUrUrGrArArArCr |
| | UrGrUrUrArUrCrArCrU |
| 1299 | rUrArArUrUrUrCrUrArCrUrCrUrUrGrUrArGrArUrArGrUrGrArUrArArCrArGr |
| | UrUrUrCrArArCrCrArA |
| 1300 | rUrArArUrUrUrCrUrArCrUrCrUrUrGrUrArGrArUrUrArArGrUrGrArUrArArCr |
| | ArGrUrUrUrCrArArCrC |
| 1301 | rUrArArUrUrUrCrUrArCrUrCrUrUrGrUrArGrArUrUrArUrArArGrUrGrArUrAr |
| | ArCrArGrUrUrUrCrArA |
| 1302 | rUrArArUrUrUrCrUrArCrUrCrUrUrGrUrArGrArUrUrArUrArUrArArGrUrGrAr |
| | UrArArCrArGrUrUrUrC |
| 1303 | rUrArArUrUrUrCrUrArCrUrCrUrUrGrUrArGrArUrUrArUrArUrArUrArArGrUr |
| | GrArUrArArCrArGrUrU |
| 1304 | rUrArArUrUrUrCrUrArCrUrCrUrUrGrUrArGrArUrUrArCrArArArGrArUrUrAr |
| | UrUrUrGrCrArCrGrArA |
| 1305 | rUrArArUrUrUrCrUrArCrUrCrUrUrGrUrArGrArUrUrArUrGrArGrUrGrGrCrUr |
| | UrCrUrGrGrGrArGrArU |
| 1306 | rUrArArUrUrUrCrUrArCrUrCrUrUrGrUrArGrArUrArGrUrGrGrCrUrUrCrUrGr |
| | GrGrArGrArUrGrGrGrU |
| 1307 | rUrArArUrUrUrCrUrArCrUrCrUrUrGrUrArGrArUrCrArGrUrGrGrUrUrUrArAr |
| | ArArArGrUrGrArCrArC |
| 1308 | rUrArArUrUrUrCrUrArCrUrCrUrUrGrUrArGrArUrGrArArGrGrUrUrUrUrUrGr |
| | UrArGrCrCrUrGrArArU |
| 1309 | rUrArArUrUrUrCrUrArCrUrCrUrUrGrUrArGrArUrArArUrGrGrArUrGrArGrCr |
| | ArGrGrGrGrArCrUrUrG |
| 1310 | rUrArArUrUrUrCrUrArCrUrCrUrUrGrUrArGrArUrUrGrUrUrGrArGrUrGrCrUr |
| | ArArGrUrGrCrGrArArC |
| 1311 | rUrArArUrUrUrCrUrArCrUrCrUrUrGrUrArGrArUrCrCrCrUrGrUrArGrUrGrUr |
| | CrCrCrArCrArArGrCrC |
| 1312 | rUrArArUrUrUrCrUrArCrUrCrUrUrGrUrArGrArUrUrCrCrCrCrUrGrUrArGrUr |
| | GrUrCrCrCrArCrArArG |
| 1313 | rUrArArUrUrUrCrUrArCrUrCrUrUrGrUrArGrArUrGrGrGrArCrArGrArArArAr |
| | GrCrArArCrUrGrGrCrA |
| 1314 | rUrArArUrUrUrCrUrArCrUrCrUrUrGrUrArGrArUrGrUrGrCrArGrUrCrArCrCr |
| | UrGrCrCrArGrCrArUrA |
| 1315 | rUrArArUrUrUrCrUrArCrUrCrUrUrGrUrArGrArUrCrUrGrGrCrArGrGrUrGrAr |
| | CrUrGrCrArCrCrArUrA |
| 1316 | rUrArArUrUrUrCrUrArCrUrCrUrUrGrUrArGrArUrArGrUrUrUrCrUrGrGrCrUr |
| | CrUrCrArCrCrArGrCrU |

All bases are in upper case Lowercase “r”″ represents RNA, 2′-hydroxy; bases not modified
by an “r”″ are DNA
All bases are linked via standard phosphodiester bonds

TABLE 16A
Exemplary AsCas12 (WT) guide RNA (DNA/RNA oligonucleotide)

| SEQ ID NO | Component | Details |
|---|---|---|
| 552 | Target sequence | TTCCTTTCCTCGTGAAGGATA |
| 610 | Targeting domain | rUrUrCrCrUrUrUrCrCrUrCrGrUrGrArArGrGrArU |
| | (corresponding RNA | rA |
| | sequence) | |
| 27 | AsCas12a stem loop | rUrArArUrUrUrCrUrArCrUrCrUrUrGrUrArGrAr |
| | (scaffold sequence) | U |
| 28 | crRNA/gRNA | rUrArArUrUrUrCrUrArCrUrCrUrUrGrUrArGrAr |
| | | UrrUrUrCrCrUrUrUrCrCrUrCrGrUrGrArArGrGrA |
| | | rUrA |
| 29 | gRNA with DNA | ATGTGTTTTTGTCAAAAGACCTTTTrUrArArU |
| | extension | rUrUrCrUrArCrUrCrUrUrGrUrArGrArUrrUrUrCr |
| | | CrUrUrUrCrCrUrCrGrUrGrArArGrGrArUrA |

All bases are in upper case
Lowercase “r” represents RNA, 2′-hydroxy; bases not modified by an “r” are DNA
All linkages between the nucleotides are standard phosphodiester groups.

TABLE 16B
Exemplary SpCas9 guide RNA (DNA/RNA oligonucleotide)

| SEQ ID NO | Component | Details |
|---|---|---|
| 61 | Target sequence | ACGTGGCGTGAACATCTGCC |
| 305 | Targeting domain | rArCrGrUrGrGrCrGrUrGrArArCrArUrCrUrGrCrC |
| | (corresponding RNA | |
| | sequence) | |
| 30 | SpCas9 stem loop | rGrUrUrUrUrArGrArGrCrUrArGrArArArUrArGr |
| | (scaffold sequence) | CrArArGrUrUrArArArArUrArArGrGrCrUrArGrU |
| | | rCrCrGrUrUrArUrCrArArCrUrUrGrArArArArAr |
| | | GrUrGrGrCrArCrCrGrArGrUrCrGrGrUrGrCrUrUr |
| | | UrU |
| 31 | crRNA/gRNA | rArCrGrUrGrGrCrGrUrGrArArCrArUrCrUrGrCrC |
| | | rGrUrUrUrUrArGrArGrCrUrArGrArArArUrArGr |
| | | CrArArGrUrUrArArArArUrArArGrGrCrUrArGrU |
| | | rCrCrGrUrUrArUrCrArArCrUrUrGrArArArArAr |
| | | GrUrGrGrCrArCrCrGrArGrUrCrGrGrUrGrCrUrUr |
| | | UrU |

All bases are in upper case
Lowercase “r” represents RNA, 2′-hydroxy; bases not modified by an “r” are DNA
All linkages between the nucleotides are standard phosphodiester groups.

In some embodiments, the targeting domain of the gRNA molecule has at least 50%, 60%, 70%, 75%, 80%, 85%, 90%, or 95% sequence identity to a nucleotide sequence set forth in SEQ ID NOs: 305-548; 607-664; 751-836; or 866-894. In some embodiments, the targeting domain of the gRNA molecule has 1, 2, 3, 4, 5, 6, 7, 8, 9, or 10 mutations relative to a nucleotide sequence set forth in SEQ ID NOs: 305-548; 607-664; 751-836; or 866-894. In some embodiments, the targeting domain of the gRNA molecule has less than 2, 3, 4, 5, 6, 7, 8, 9, or 10 mutations relative to a nucleotide sequence set forth in SEQ ID NOs: 305-548; 607-664; 751-836; or 866-894. In some embodiments, the first gRNA molecule targeting domain and the second gRNA molecule targeting domain comprise, a first 5′ targeting domain, and a second 3′ targeting domain as set forth in Table 17A-17B.

TABLE 17A
5′ and 3′ targeting domain sequences
for targeting LDLR with SpCas9

| 5′ Targeting Domain | 3′ Targeting Domain |
|---|---|
| SEQ ID NO: | SEQ ID NO: |
| 305 | 119 |
| 306 | 123 |
| 307 | 188 |
| 308 | 275 |
| 314 | 277 |
| 315 | 280 |
| 316 | 282 |
| 317 | 283 |
| | 284 |
| | 285 |
| | 286 |
| | 287 |
| | 288 |
| | 291 |
| | 292 |
| | 293 |
| | 296 |
| | 299 |
| | 300 |
| | 301 |

TABLE 17B
5′ and 3′ targeting domain sequences
for targeting LDLR with AsCas12a

| 5′ Targeting Domain | 3′ Targeting Domain |
|---|---|
| SEQ ID NO: | SEQ ID NO: |
| 610 | 643 |
| 611 | 647 |
| 634 | |
| 637 | |

### 4.1 Guide RNA Modifications

The activity, stability, or other characteristics of gRNAs can be altered through the incorporation of certain modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not wishing to be bound by theory it is also believed that certain modified gRNAs described herein can exhibit a reduced innate immune response when introduced into cells. Those of skill in the art will be aware of certain cellular responses commonly observed in cells, e.g., mammalian cells, in response to exogenous nucleic acids, particularly those of viral or bacterial origin. Such responses, which can comprise induction of cytokine expression and release and cell death, can be reduced or eliminated altogether by the modifications presented herein.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cas12a gRNA, and/or a targeting domain of a gRNA. In addition, the entire contents of WO2017136794, WO2017004279, WO2018107028, Yin, et al. Nat Biotechnol 35 (12): 1179-1187 (2017) and Finn, et al. Cell Rep 22 (9): 2227-2235 (2018) are expressly incorporated in their entirety.

As one example, the 5′ end of a gRNA can comprise a eukaryotic mRNA cap structure or cap analog (e.g., a G(5′)ppp(5′)G cap analog, a m7G(5′)ppp(5′)G cap analog, or a 3′-O-Me-m7G(5′)ppp(5′)G anti reverse cap analog (ARCA)), as shown below:

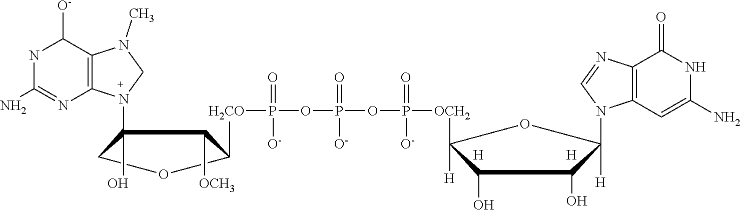

The cap or cap analog can be included during either chemical synthesis or in vitro transcription of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another modification involves the addition, at the 3′ end of a gRNA, of a plurality (e.g., 1-10, 10-20, or 25-200) of adenine (A) residues referred to as a polyA tract or sequence (PAS). The polyA tract can be added to a gRNA during chemical synthesis, following in vitro transcription using a polyadenosine polymerase (e.g., E. coli Poly(A) Polymerase), or in vivo by means of a polyadenylation sequence, as described in Maeder.

It should be noted that the modifications described herein can be combined in any suitable manner, e.g., a gRNA, whether transcribed in vivo from a DNA vector, or in vitro transcribed gRNA, can comprise either or both of a 5′ cap structure or cap analog and a 3′ polyA tract.

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

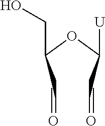

wherein “U” can be an unmodified or modified uridine.

The 3′ terminal U ribose can be modified with a 2′3′ cyclic phosphate as shown below:

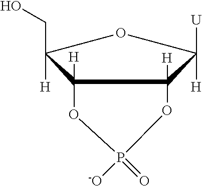

wherein “U” can be an unmodified or modified uridine.

Guide RNAs can contain 3′ nucleotides which can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into the gRNA, e.g., wherein the 2′ OH— group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F (as illustrated in FIG. 3) or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also comprise “locked” nucleic acids (LNA) in which the 2′ OH— group can be connected, e.g., by a C1-6 alkylene or C1-6 heteroalkylene bridge, to the 4′ carbon of the same ribose sugar. Any suitable moiety can be used to provide such bridges, which may comprise without limitation methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclyl, arylamino, diarylamino, heteroarylamino, or diheteroarylamino, ethylenediamine, or polyamino).

In certain embodiments, a gRNA can comprise a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with a-L-threofuranosyl-(3′->2′)).

Generally, gRNAs comprise the sugar group ribose, which is a 5-membered ring with an oxygen atom. Exemplary modified gRNAs can comprise, without limitation, replacement of the oxygen in ribose (e.g., with sulfur(S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, O- and N-alkylated nucleotides, e.g., N6-methyl adenosine, can be incorporated into the gRNA. In certain embodiments, one or more or all of the nucleotides in a gRNA are deoxynucleotides.

In certain embodiments, a gRNA comprises one or more 2′F modifications. In certain embodiments, a 2′F modification is positioned on a nucleotide internal to the gRNA (which may be represented herein as “i2F”). In certain embodiments, a gRNA comprises multiple 2′F modifications in an aggressive pattern or a conservative pattern. In certain embodiments, a gRNA hairpin region has the same sequence as the gRNA scaffold sequence. For example, a Cas12a gRNA can have hairpin sequence of SEQ ID NO: 33 and a scaffold sequence of SEQ ID NO: 33 (Table 18B). Herein a Cas12a gRNA with an “aggressive” pattern of hairpin 2′F modifications comprises a hairpin comprising 2′F modifications at each of nucleotide positions 7 and 8 of the hairpin. In certain embodiments, a gRNA comprising an aggressive pattern of hairpin 2′F modifications comprises a hairpin of SEQ ID NO: 37. Herein a Cas12a gRNA with a “conservative” pattern of hairpin 2′F modifications comprises a hairpin lacking 2′F modifications at each of nucleotide positions 7 and 8 of the hairpin. In certain embodiments, a gRNA comprising a conservative pattern of hairpin 2′F modifications comprises a hairpin of SEQ ID NO: 34.

In certain embodiments, gRNAs as used herein may be modified or unmodified gRNAs. In certain embodiments, a gRNA may include one or more modifications. In certain embodiments, the one or more modifications may include a phosphorothioate linkage modification, a phosphorodithioate (PS2) linkage modification, a 2′-O-methyl modification, or combinations thereof. In certain embodiments, the one or more modifications may be at the 5′ end of the gRNA, at the 3′ end of the gRNA, or combinations thereof. In general, any combination of modifications may be used. In non-limiting examples, patterns of modification comprising, a combination of 5′ extension; 5′3′ idT; hairpin 2′F in an aggressive pattern (see, e.g., the relevant gRNAs in Table 18A-18B for illustration of an exemplary hairpin_2′F aggressive pattern); a combination of 5′3′ idT and hairpin 2′F in an aggressive pattern; or a combination of 5′3′ idT and 5′ extension combination can be used. The pattern of modification of the gRNA, e.g., the modifications illustrated in FIG. 3 or described elsewhere herein, can be applied to gRNAs with any targeting domain (e.g., targeting LDLR) and/or for targeting genes in a tissue specific manner.

### 5.1 Cas9

Crystal structures have been determined for S. pyogenes Cas9 (Jinek 2014), and for S. aureus Cas9 in complex with a unimolecular guide RNA and a target DNA (Nishimasu 2014; Anders 2014; and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g., a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA: DNA recognition, whereas the REC domain is thought to interact with the repeat: anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e., bottom) strand of the target nucleic acid. It can be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e., top) strand of the target nucleic acid. The PI domain, as its name suggests, contributes to PAM specificity.

While certain functions of Cas9 are linked to (but not necessarily fully determined by) the specific domains set forth above, these and other functions can be mediated or influenced by other Cas9 domains, or by multiple domains on either lobe. For instance, in S. pyogenes Cas9, as described in Nishimasu 2014, the repeat: antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains).

TABLE 18A
Exemplary Cas9 gRNA modification pattern*

| Modification | gRNA sequence |
|---|---|
| Hairpin region 1 | GUUUUAGAGCUAGAAAUAGCAAGUUAAAAUAAGGCUA |
| | GUCCGUUAUCAACUUGAAAAAGUGGCACCGAGUCGGUG |
| | CUUUU [SEQ ID NO: 30] |
| Exemplary Targeting | 12345678901234567890 |
| region | |
| Exemplary conservative | GUUUUAGAGCUAGAAAUAGCAAGUUAAAAUAAGGCUA |
| modified pattern | GUCCGUUAUCAACUUGAAAAAGUGGCACCGAGUCGGU |
| (hairpin) | GCU*U*U*U [SEQ ID NO: 1360] |
| Exemplary conservative | 12345678901234567890GUUUUAGAGCUAGAAAUAGCAAG |
| modified pattern | UUAAAAUAAGGCUAGUCCGUUAUCAACUUGAAAAAGU |
| (targeting region + | GGCACCGAGUCGGUGCU*U*U*U [SEQ ID NO: 1361] |
| hairpin) | |
| Exemplary conservative | 1*2*3*45678901234567890GUUUUAGAGCUAGAAAUAGCA |
| modified pattern | AGUUAAAAUAAGGCUAGUCCGUUAUCAACUUGAAAAA |
| (targeting region + | GUGGCACCGAGUCGGUGCU*U*U*U [SEQ ID NO: 1362] |
| hairpin) | |
| Exemplary aggressive | GUUUUAGAGCUAGAAAUAGCAAGUUAAAAUAAGGCUA |
| modified pattern | GUCCGUUAUCAAC*U*U*G*A*A*A*A*A*GUGG*CA*C*C* |
| (hairpin) | G*A*G*U*CG*G*U*G*C*U*U*U*U*U [SEQ ID NO: 1363] |
| Exemplary aggressive | 12345678901234567890GUUUUAGAGCUAGAAAUAGCAAG |
| modified pattern | UUAAAAUAAGGCUAGUCCGUUAUCAAC*U*U*G*A*A*A* |
| (targeting region + | A*A*GUGG*CA*C*C*G*A*G*U*CG*G*U*G*C*U*U*U* |
| hairpin) | U*U [SEQ ID NO: 1364] |
| Exemplary aggressive | 1*2*3*45*6*7*8*9*01234567890GUUUUAGAGCUAGAAAUA |
| 2′F pattern (targeting | GCAAGUUAAAAUAAGGCUAGUCCGUUAUCAAC*U*U*G* |
| region + hairpin) | A*A*A*A*A*GUGG*CA*C*C*G*A*G*U*CG*G*U*G*C*U*U* |
| | U*U*U [SEQ ID NO: 32] |

*Underlined = 2′ F modified nucleotide; Bolded = 2′ OMe modified nucleotide; No underline or bold = unmodified nucleotide; Phosphorothioate bonds are indicated by *.

TABLE 18B
Exemplary Cas12a gRNA modification pattern*

| Modification | gRNA sequence |
|---|---|
| Hairpin region | UAAUUUCUACUCUUGUAGAU |
| | [SEQ ID NO: 33] |
| Exemplary Targeting | 123456789012345678901 |
| region | |
| Exemplary conservative | UAAUUUCUACUCUUGUAGAU |
| 2′F pattern (hairpin) | [SEQ ID NO: 34] |
| Exemplary conservative | UAAUUUCUACUCUUGUAGAU |
| 2′F pattern (hairpin + | 123456789012345678901 |
| targeting region | [SEQ ID NO: 35] |
| Exemplary conservative | UAAUUUCUACUCUUGUAGAU |
| 2′F pattern (targeting | 123456789012345678901 |
| region) | [SEQ ID NO: 36] |
| Exemplary aggressive | UAAUUUCUACUCUUGUAGAU |
| 2′F pattern (hairpin) | [SEQ ID NO: 37] |
| Exemplary aggressive | UAAUUUCUACUCUUGUAGAU |
| 2′F pattern (hairpin + | 123456789012345678901 |
| targeting region) | [SEQ ID NO: 38] |
| Exemplary aggressive | UAAUUUCUACUCUUGUAGAU |
| 2′F pattern (targeting | 123456789012345678901 |
| region) | [SEQ ID NO: 39] |

*Underlined = 2′ F modified nucleotide; No underline = unmodified nucleotide

In certain embodiments, a modified gRNA as described herein comprises one or more modifications of the gRNA in the hairpin region, the targeting domain, or both. For example, but not by way of limitation, the hairpin region of such a modified gRNA can comprise SEQ ID NO: 33. In certain embodiments, the hairpin region of a modified gRNA comprises one or more 2′Fluorine modifications (e.g., see SEQ ID NOs: 34 or 37 in Table 18B). In certain embodiments, the hairpin region of a modified gRNA comprises a DNA extension at the 5′ end of the hairpin region. In certain embodiments, the hairpin region of a modified gRNA comprises one or more 2′O-methyl modifications. For example, in certain embodiments, a modified gRNA comprises a 1xPSOMe modification on a 5′ terminus and/or a 3′ terminus; in certain embodiments, a modified gRNA comprises a 3xPSOMe modification on a 5′ terminus and/or a 3′ terminus. In certain embodiments, the hairpin region of a modified gRNA comprises one or more 5′ inverted dT modifications. In certain embodiments, the hairpin region of a modified gRNA comprises one or more 3′ inverted dT modifications. In certain embodiments, the hairpin region of a modified gRNA comprises a 3′ or 5′ pseudoknot.

In certain embodiments, the hairpin region of a modified gRNA comprises a 3′ pseudoknot. In certain embodiments, the hairpin region of a modified gRNA comprises a locked nucleic acid (LNA). In certain embodiments, the hairpin region of a modified gRNA comprises a LNA with a 5′ extension.

In certain embodiments, the hairpin region of a modified gRNA can comprise one or more of 5′ extensions, 2′Fluorine modifications, 2′O-methyl modifications, 5′ inverted dT modifications, or 3′ inverted dT modifications, a pseudoknot, or an LNA. For example, in certain embodiments, the hairpin region of a modified gRNA comprises a 5′ extension and a 1xPSOMe modification on 5′ and 3′ ends. In certain embodiments, the hairpin region of a modified gRNA comprises a 1xPSOMe modification on the 5′ end and a 3′ pseudoknot. In certain embodiments, the hairpin region of the modified gRNAs comprises a 5′ extension and 1xPSOMe modification on the 3′ end only. In certain embodiments, the hairpin region of a modified gRNA comprises a 5′ extension and a conservative pattern of 2′F modifications. In certain embodiments, the hairpin region of the modified gRNAs comprises a 5′ extension and an aggressive pattern of 2′F modifications. In certain embodiments, a modified gRNA comprises a hairpin with a 5′ extension and inverted dT modifications at the 5′ and 3′ termini. In certain embodiments, a modified gRNA comprises a hairpin with a 5′ extension, inverted dT modifications at the 5′ and 3′ termini, and an LNA. In certain embodiments, a modified gRNA comprises a hairpin with a 5′ extension and an aggressive pattern of 2′ modifications, and 1xPSOMe modifications on 5′ and 3′ termini. In certain embodiments, a modified gRNA comprises a hairpin with a 5′ extension and an aggressive pattern of 2′F modifications, and inverted dT modifications at the 5′ and 3′ termini. In certain embodiments, the hairpin region of a modified gRNA comprises a 5′ extension and a 2′OMe modification.

In certain embodiments, the targeting domain of the gRNA can comprise one or more of 5′ extensions, 2′Fluorine modifications, 2′O-methyl modifications, 5′ inverted dT modifications, or 3′ inverted dT modifications, a pseudoknot, or an LNA. In certain embodiments the targeting domain of a gRNA comprises 2′F modifications at nucleotide positions 1, 8, 9, 10, 11, 12, 17, 19 and optionally 20 and 21. In certain embodiments the targeting domain of a gRNA comprises 2′F modifications at nucleotide positions 1, 2, 3, 7, 8, 9, 10, 11, 12, 14, 15, 17, 19 and optionally 20 and 21. In certain embodiments, the targeting domain of a gRNA comprises one or more of 2′Fluorine modifications, 2′O-methyl modifications, 5′ inverted dT modifications, or 3′ inverted dT modifications.

In any of the above embodiments, a gRNA can comprise any pattern of 2′F modifications in the hairpin (e.g., a conservative pattern or an aggressive pattern) and any pattern of 2′F modifications in the targeting domain (e.g., 2′F modifications at nucleotide positions 1, 8, 9, 10, 11, 12, 17, 19 of the targeting domain; 2′F modifications at nucleotide positions 1, 2, 3, 7, 8, 9, 10, 11, 12, 14, 15, 17, 19 of the targeting domain; or no 2′F modifications in the targeting domain).

In certain embodiments, a genome editing system described herein comprises a gRNA comprising an RNA portion comprising a 2′F modification. In certain embodiments, the RNA portion comprises a 5′ hairpin and a 3′ targeting domain. In certain embodiments, a gRNA further comprises an extension region at the 5′ end of the hairpin (e.g., a DNA extension). In certain embodiments, a gRNA comprises a hairpin comprising a 2′F modification at one or more of nucleotide positions 1, 5, 6, 7, 8, 9, 10, 12, 13, 14, 16, 17, 18 or 19 of the hairpin (i.e., counting from the 5′ end of the hairpin, e.g., having SEQ ID NO: 33). In certain embodiments, a gRNA comprises a hairpin comprising multiple 2′F modifications. In certain embodiments, a gRNA comprises a hairpin comprising 2′F modifications at two or more, three or more, four or more, five or more, six or more, seven or more, eight or more, nine or more, or ten or more of nucleotide positions 1, 5, 6, 7, 8, 9, 10, 12, 13, 14, 16, 17, 18 or 19 of the hairpin. In certain embodiments, a gRNA comprises a hairpin comprising 2′F modifications at nucleotide positions 1, 5, 6, 7, 8, 9, 10, 12, 13, 14, 16, 17, 18 and 19 of the hairpin. In certain embodiments, a gRNA comprises a hairpin with a pattern of 2′F modifications that consists of 2′F modifications at nucleotide positions 1, 5, 6, 7, 8, 9, 10, 12, 13, 14, 16, 17, 18 and 19 of the hairpin (exemplifying an aggressive pattern of 2′F modifications). In certain embodiments, a gRNA comprises a hairpin that does not include a 2′F modification on at least one of nucleotide positions 7 or 8 of the hairpin. In certain embodiments, a gRNA comprises a hairpin that lacks a 2′F modification at each of nucleotide positions 7 and 8 of the hairpin. In certain embodiments, a gRNA comprises a hairpin comprising 2′F modifications at nucleotide positions 1, 5, 6, 9, 10, 12, 13, 14, 16, 17, 18 and 19 of the hairpin. In certain embodiments, a gRNA comprises a hairpin with a pattern of 2′F modifications that consists of 2′F modifications at nucleotide positions 1, 5, 6, 9, 10, 12, 13, 14, 16, 17, 18 and 19 of the hairpin (exemplifying a conservative pattern of 2′F modifications). In certain embodiments, a gRNA comprises a hairpin that lacks a 2′F modification at one or more of nucleotide positions 2, 3, 4, 11 and 15 of the hairpin. In certain embodiments, a gRNA comprises a hairpin that lacks a 2′F modification at each of nucleotide positions 2, 3, 4, 11 and 15 of the hairpin.

### 5. RNA-Guided Nucleases

RNA-guided nucleases according to the present disclosure comprise, but are not limited to, naturally-occurring Class 2 CRISPR nucleases such as Cas9, and Cas12a, as well as other nucleases derived or obtained therefrom. In functional terms, RNA-guided nucleases are defined as those nucleases that: (a) interact with (e.g. complex with) a gRNA; and (b) together with the gRNA, associate with, and optionally cleave or modify, a target region of a DNA that comprises (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations can exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cas12a), species (e.g., S. pyogenes vs. S. aureus) or variation (e.g., full-length vs. truncated or split; naturally occurring PAM specificity vs. engineered PAM specificity, etc.) of RNA-guided nuclease.

The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. For example, Cas9 nucleases recognize PAM sequences that are 3′ of the protospacer, while Cas12a, on the other hand, generally recognizes PAM sequences that are 5′ of the protospacer.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cas12a recognizes a TTN PAM sequence. PAM sequences have been identified for a variety of RNA-guided nucleases, and a strategy for identifying novel PAM sequences has been described by Shmakov et al., 2015, Molecular Cell 60, 385-397, Nov. 5, 2015. It should also be noted that engineered RNA-guided nucleases can have PAM specificities that differ from the PAM specificities of reference molecules (for instance, in the case of an engineered RNA-guided nuclease, the reference molecule can be the naturally occurring variant from which the RNA-guided nuclease is derived, or the naturally occurring variant having the greatest amino acid sequence homology to the engineered RNA-guided nuclease).

In addition to their PAM specificity, RNA-guided nucleases can be characterized by their DNA cleavage activity: naturally-occurring RNA-guided nucleases typically form DSBs in target nucleic acids but engineered variants have been produced that generate only SSBs (discussed above) Ran & Hsu, et al., Cell 154 (6), 1380-1389 Sep. 12, 2013 (Ran), incorporated by reference herein), or that that do not cut at all.

5.2 Cas12a (formerly known as Cpf1)

The crystal structure of Acidaminococcus sp. Cas12a in complex with crRNA and a double-stranded (ds) DNA target including a TTTN PAM sequence has been solved by Yamano et al. (Cell. 2016 May 5; 165 (4): 949-962 (Yamano), incorporated by reference herein). Cas12a, like Cas9, has two lobes: a REC (recognition) lobe, and a NUC (nuclease) lobe. The REC lobe comprises REC1 and REC2 domains, which lack similarity to any known protein structures. The NUC lobe, meanwhile, comprises three RuvC domains (RuvC-I, -II and —III) and a BH domain. However, in contrast to Cas9, the Cas12a REC lobe lacks an HNH domain, and comprises other domains that also lack similarity to known protein structures: a structurally unique PI domain, three Wedge (WED) domains (WED-I, -II and-III), and a nuclease (Nuc) domain.

While Cas9 and Cas12a share similarities in structure and function, it should be appreciated that certain Cas12a activities are mediated by structural domains that are not analogous to any Cas9 domains. For instance, cleavage of the complementary strand of the target DNA appears to be mediated by the Nuc domain, which differs sequentially and spatially from the HINH domain of Cas9. Additionally, the non-targeting portion of Cas12a gRNA (the handle) adopts a pseudoknot structure, rather than a stem loop structure formed by the repeat: antirepeat duplex in Cas9 gRNAs.

Non-limiting examples of RNA-guided nucleases include, Cas9 (e.g., SpCas9, SaCas9, (KKH) SaCas9, eSpCas9, Cas9-HF1, HypaCas9, dCas9-Fokl, Sniper-Cas9, xCas9, evoCas9, SpCas9-NG, VRQR, VRER, NmeCas9, CjCas9), Cas12a (also known as Cpf1; e.g., AsCas12a, LbCas12a), Cas12b (e.g., AaCas12b, BhCas12b, BhCas12bV4), Cas12c (e.g., Cas12cl, Cas12c2), Cas12h (e.g., Cas12 h1), Cas12i (e.g., Cas12i1), CasX, CasY, and CasΦ.

### 5.3 Modifications of RNA-Guided Nucleases

The RNA-guided nucleases described above have activities and properties that can be useful in a variety of applications, but the skilled artisan will appreciate that RNA-guided nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that can be made in the RuvC domains, in the Cas9 HNH domain, or in the Cas12a Nuc domain are described in Ran and Yamano, as well as in Cotta-Ramusino. In general, mutations that reduce or eliminate activity in one of the two nuclease domains result in RNA-guided nucleases with nickase activity, but it should be noted that the type of nickase activity varies depending on which domain is inactivated. As one example, inactivation of a RuvC domain or of a Cas9 HNH domain results in a nickase.

Modifications of PAM specificity relative to naturally-occurring Cas9 reference molecules have been described by Kleinstiver et al. for both S. pyogenes (Kleinstiver et al., Nature. 2015 Jul. 23; 523 (7561): 481-5 (Kleinstiver I)) and S. aureus (Kleinstiver et al., Nat Biotechnol. 2015 December; 33 (12): 1293-1298 (Klienstiver II)). Kleinstiver et al. have also described modifications that improve the targeting fidelity of Cas9 (Nature, 2016 Jan. 28; 529, 490-495 (Kleinstiver III)). Each of these references is incorporated by reference herein.

RNA-guided nucleases have been split into two or more parts, as described by Zetsche et al. (Nat Biotechnol. 2015 February; 33 (2): 139-42 (Zetsche II), incorporated by reference), and by Fine et al. (Sci Rep. 2015 Jul. 1; 5:10777 (Fine), incorporated by reference).

RNA-guided nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger et al., Nature Biotechnology 32, 577-582 (2014), which is incorporated by reference for all purposes herein.

RNA-guided nucleases also optionally comprise a tag, such as, but not limited to, a nuclear localization signal (NLS) to facilitate movement of RNA-guided nuclease protein into the nucleus. In certain embodiments, the RNA-guided nuclease can incorporate C- and/or N-terminal nuclear localization signals, e.g., SEQ ID NO: 40, presented herein. Nuclear localization sequences are known in the art and are described in Maeder and elsewhere.

The foregoing list of modifications is intended to be exemplary in nature, and the skilled artisan will appreciate, in view of the instant disclosure, that other modifications can be possible or desirable in certain applications. For brevity, therefore, exemplary systems, methods and compositions of the present disclosure are presented with reference to particular RNA-guided nucleases, but it should be understood that the RNA-guided nucleases used can be modified in ways that do not alter their operating principles. Such modifications are within the scope of the present disclosure.

Exemplary suitable nuclease variants comprise, but are not limited to, AsCas12a variants comprising an M537R substitution, an H800A substitution, and/or an F870L substitution, or any combination thereof (numbering scheme according to AsCas12a wild-type sequence). Exemplary suitable nuclease variants comprise, but are not limited to, SpCas9 variants comprising an D10A substitution, and/or an H840A substitution, (numbering scheme according to SpCas9 wild-type sequence). The entire contents of WO2019/067872 are expressly incorporated by reference herein in their entirety. Other suitable modifications of the AsCas12a amino acid sequence are known to those of ordinary skill in the art. Some non-limiting exemplary sequences of wild-type Cas9, Cas9 variants, wild-type AsCas12a, and AsCas12a variants are as follows:

5.3.1 Cas9 amino acid sequences

SpCas9 amino acid sequence
[SEQ ID NO: 1338]
MDKKYSIGLDIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRS
DKNRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGD
sNLS-SpCas9-NLS-HA variant 1 amino acid sequence:
[SEQ ID NO: 1339]
MAPKKKRKVGIHGVPAADKKYSIGLDIGTNSVGWAVITDEYKVPSKKFKVLGNTDR
HSIKKNLIGALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTDKADLRLIYL
ALAHMIKFRGHFLIEGDLNPDNSDVDKLFIQLVQTYNQLFEENPINASGVDAKAILSA
RLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQ
DLTLLKALVRQQLPEKYKEIFFDQSKNGYAGYIDGGASQEEFYKFIKPILEKMDGTEE
LLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVT
VKQLKEDYFKKIECFDSVEISGVEDRFNASLGTYHDLLKIIKDKDFLDNEENEDILEDI
VLTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSPAI
KKGILQTVKVVDELVKVMGRHKPENIVIEMARENQTTQKGQKNSRERMKRIEEGIK
ELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIREVKVITL
KSKLVSDFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIV
WDKGRDFATVRKVLSMPQVNIVKKTEVQTGGFSKESILPKRNSDKLIARKKDWDPK
KYGGFDSPTVAYSVLVVAKVEKGKSKKLKSVKELLGITIMERSSFEKNPIDFLEAKG
YKEVKKDLIIKLPKYSLFELENGRKRMLASAGELQKGNELALPSKYVNFLYLASHYE
KLKGSPEDNEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQSITGLYETRID
LSQLGGDSGGKRPAATKKAGQAKKKKGSYPYDVPDYA
SpCas9-sNLS variant 2 amino acid sequence:
[SEQ ID NO: 1340]
MDKKYSIGLDIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRS
DKNRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGDGGGSPKK
KRKV
SpCas9-sNLS D10A variant 3 amino acid sequence:
[SEQ ID NO: 1341]
MDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRS
DKNRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGDGGGSPKK
KRKV
SpCas9-sNLS D10A H840A variant 4 amino acid sequence:
[SEQ ID NO: 1342]
MDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENT
QLQNEKLYLYYLQNGRDMYVDQELDINRLSDYDVDAIVPQSFLKDDSIDNKVLTRS
DKNRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGDGGGSPKK
KRKV
SpCas9 D10A variant 5 amino acid sequence:
[SEQ ID NO: 1343]
MDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRS
DKNRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGD
SpCas9 D10A H840A variant 6 amino acid sequence:
[SEQ ID NO: 1344]
MDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENT
QLQNEKLYLYYLQNGRDMYVDQELDINRLSDYDVDAIVPQSFLKDDSIDNKVLTRS
DKNRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGD
SpCas9-sNLS-sNLS variant 7 amino acid sequence:
[SEQ ID NO: 1345]
MDKKYSIGLDIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRS
DKNRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGDGSGSPKK
KRKVDGSPKKKRKVDSG
SpCas9-sNLS-sNLS D10A variant 8 amino acid sequence:
[SEQ ID NO: 1346]
MDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRS
DKNRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGDGSGSPKK
KRKVDGSPKKKRKVDSG
SpCas9-sNLS-sNLS D10A H840A variant 9 amino acid sequence:
[SEQ ID NO: 1347]
MDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENT
QLQNEKLYLYYLQNGRDMYVDQELDINRLSDYDVDAIVPQSFLKDDSIDNKVLTRS
DKNRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGDGSGSPKK
KRKVDGSPKKKRKVDSG
5.3.2 Cas12a amino acid sequences
His-AsCpf1-sNLS-sNLS H800A amino acid sequence:
[SEQ ID NO: 40]
MGHHHHHHGSTQFEGFTNLYQVSKTLRFELIPQGKTLKHIQEQGFIEEDKARNDHYK
ELKPIIDRIYKTYADQCLQLVQLDWENLSAAIDSYRKEKTEETRNALIEEQATYRNAI
HDYFIGRTDNLTDAINKRHAEIYKGLFKAELFNGKVLKQLGTVTTTEHENALLRSFD
KFTTYFSGFYENRKNVFSAEDISTAIPHRIVQDNFPKFKENCHIFTRLITAVPSLREHFE
NVKKAIGIFVSTSIEEVFSFPFYNQLLTQTQIDLYNQLLGGISREAGTEKIKGLNEVLNL
AIQKNDETAHIIASLPHRFIPLFKQILSDRNTLSFILEEFKSDEEVIQSFCKYKTLLRNEN
VLETAEALFNELNSIDLTHIFISHKKLETISSALCDHWDTLRNALYERRISELTGKITKS
AKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNY
ATKKPYSVEKFKLNFQMPTLASGWDVNKEKNNGAILFVKNGLYYLGIMPKQKGRY
KALSFEPTEKTSEGFDKMYYDYFPDAAKMIPKCSTQLKAVTAHFQTHTTPILLSNNFI
EPLEITKEIYDLNNPEKEPKKFQTAYAKKTGDQKGYREALCKWIDFTRDFLSKYTKT
TSIDLSSLRPSSQYKDLGEYYAELNPLLYHISFQRIAEKEIMDAVETGKLYLFQIYNKD
FAKGHHGKPNLHTLYWTGLFSPENLAKTSIKLNGQAELFYRPKSRMKRMAARLGEK
MLNKKLKDQKTPIPDTLYQELYDYVNHRLSHDLSDEARALLPNVITKEVSHEIIKDR
RFTSDKFFFHVPITLNYQAANSPSKFNQRVNAYLKEHPETPIIGIDRGERNLIYITVIDS
TGKILEQRSLNTIQQFDYQKKLDNREKERVAARQAWSVVGTIKDLKQGYLSQVIHEI
VDLMIHYQAVVVLENLNFGFKSKRTGIAEKAVYQQFEKMLIDKLNCLVLKDYPAEK
VGGVLNPYQLTDQFTSFAKMGTQSGFLFYVPAPYTSKIDPLTGFVDPFVWKTIKNHE
SRKHFLEGFDFLHYDVKTGDFILHFKMNRNLSFQRGLPGFMPAWDIVFEKNETQFDA
KGTPFIAGKRIVPVIENHRFTGRYRDLYPANELIALLEEKGIVFRDGSNILPKLLENDD
SHAIDTMVALIRSVLQMRNSNAATGEDYINSPVRDLNGVCFDSRFQNPEWPMDADA
NGAYHIALKGQLLLNHLKESKDLKLQNGISNQDWLAYIQELRNGSPK
KKRKVGSPKKKRKV
AsCas12a variant 1 amino acid sequence:
[SEQ ID NO: 41]
MTQFEGFTNLYQVSKTLRFELIPQGKTLKHIQEQGFIEEDKARNDHYKELKPIIDRIYK
TYADQCLQLVQLDWENLSAAIDSYRKEKTEETRNALIEEQATYRNAIHDYFIGRTDN
LTDAINKRHAEIYKGLFKAELFNGKVLKQLGTVTTTEHENALLRSFDKFTTYFSGFYE
NRKNVFSAEDISTAIPHRIVQDNFPKFKENCHIFTRLITAVPSLREHFENVKKAIGIFVS
TSIEEVFSFPFYNQLLTQTQIDLYNQLLGGISREAGTEKIKGLNEVLNLAIQKNDETAH
IIASLPHRFIPLFKQILSDRNTLSFILEEFKSDEEVIQSFCKYKTLLRNENVLETAEALFN
ELNSIDLTHIFISHKKLETISSALCDHWDTLRNALYERRISELTGKITKSAKEKVQRSL
KHEDINLQEIISAAGKELSEAFKQKTSEILSHAHAALDQPLPTTLKKQEEKEILKSQLD
SLLGLYHLLDWFAVDESNEVDPEFSARLTGIKLEMEPSLSFYNKARNYATKKPYSVE
KFKLNFQRPTLASGWDVNKEKNNGAILFVKNGLYYLGIMPKQKGRYKALSFEPTEK
TSEGFDKMYYDYFPDAAKMIPKCSTQLKAVTAHFQTHTTPILLSNNFIEPLEITKEIYD
LNNPEKEPKKFQTAYAKKTGDQKGYREALCKWIDFTRDFLSKYTKTTSIDLSSLRPSS
QYKDLGEYYAELNPLLYHISFQRIAEKEIMDAVETGKLYLFQIYNKDFAKGHHGKPN
LHTLYWTGLFSPENLAKTSIKLNGQAELFYRPKSRMKRMAHRLGEKMLNKKLKDQ
KTPIPDTLYQELYDYVNHRLSHDLSDEARALLPNVITKEVSHEIIKDRRFTSDKFLFHV
PITLNYQAANSPSKFNQRVNAYLKEHPETPIIGIDRGERNLIYITVIDSTGKILEQRSLN
TIQQFDYQKKLDNREKERVAARQAWSVVGTIKDLKQGYLSQVIHEIVDLMIHYQAV
VVLENLNFGFKSKRTGIAEKAVYQQFEKMLIDKLNCLVLKDYPAEKVGGVLNPYQL
TDQFTSFAKMGTQSGFLFYVPAPYTSKIDPLTGFVDPFVWKTIKNHESRKHFLEGFDF
LHYDVKTGDFILHFKMNRNLSFQRGLPGFMPAWDIVFEKNETQFDAKGTPFIAGKRI
VPVIENHRFTGRYRDLYPANELIALLEEKGIVFRDGSNILPKLLENDDSHAIDTMVALI
RSVLQMRNSNAATGEDYINSPVRDLNGVCFDSRFQNPEWPMDADANGAYHIALKG
QLLLNHLKESKDLKLQNGISNQDWLAYIQELRNGRSSDDEATADSQHAAPPKKKRK
VGGSGGSGGSGGSGGSGGSGGSGGSLEHHHHHH
AsCas12a variant 2 amino acid sequence:
[SEQ ID NO: 42]
MTQFEGFTNLYQVSKTLRFELIPQGKTLKHIQEQGFIEEDKARNDHYKELKPIIDRIYK
TYADQCLQLVQLDWENLSAAIDSYRKEKTEETRNALIEEQATYRNAIHDYFIGRTDN
LTDAINKRHAEIYKGLFKAELFNGKVLKQLGTVTTTEHENALLRSFDKFTTYFSGFYE
NRKNVFSAEDISTAIPHRIVQDNFPKFKENCHIFTRLITAVPSLREHFENVKKAIGIFVS
TSIEEVFSFPFYNQLLTQTQIDLYNQLLGGISREAGTEKIKGLNEVLNLAIQKNDETAH
IIASLPHRFIPLFKQILSDRNTLSFILEEFKSDEEVIQSFCKYKTLLRNENVLETAEALFN
ELNSIDLTHIFISHKKLETISSALCDHWDTLRNALYERRISELTGKITKSAKEKVQRSL
KHEDINLQEIISAAGKELSEAFKQKTSEILSHAHAALDQPLPTTLKKQEEKEILKSQLD
SLLGLYHLLDWFAVDESNEVDPEFSARLTGIKLEMEPSLSFYNKARNYATKKPYSVE
KFKLNFQMPTLASGWDVNKEKNNGAILFVKNGLYYLGIMPKQKGRYKALSFEPTEK
TSEGFDKMYYDYFPDAAKMIPKCSTQLKAVTAHFQTHTTPILLSNNFIEPLEITKEIYD
LNNPEKEPKKFQTAYAKKTGDQKGYREALCKWIDFTRDFLSKYTKTTSIDLSSLRPSS
QYKDLGEYYAELNPLLYHISFQRIAEKEIMDAVETGKLYLFQIYNKDFAKGHHGKPN
LHTLYWTGLFSPENLAKTSIKLNGQAELFYRPKSRMKRMAHRLGEKMLNKKLKDQ
KTPIPDTLYQELYDYVNHRLSHDLSDEARALLPNVITKEVSHEIIKDRRFTSDKFFFHV
PITLNYQAANSPSKFNQRVNAYLKEHPETPIIGIDRGERNLIYITVIDSTGKILEQRSLN
TIQQFDYQKKLDNREKERVAARQAWSVVGTIKDLKQGYLSQVIHEIVDLMIHYQAV
VVLENLNFGFKSKRTGIAEKAVYQQFEKMLIDKLNCLVLKDYPAEKVGGVLNPYQL
TDQFTSFAKMGTQSGFLFYVPAPYTSKIDPLTGFVDPFVWKTIKNHESRKHFLEGFDF
LHYDVKTGDFILHFKMNRNLSFQRGLPGFMPAWDIVFEKNETQFDAKGTPFIAGKRI
VPVIENHRFTGRYRDLYPANELIALLEEKGIVFRDGSNILPKLLENDDSHAIDTMVALI
RSVLQMRNSNAATGEDYINSPVRDLNGVCFDSRFQNPEWPMDADANGAYHIALKG
QLLLNHLKESKDLKLQNGISNQDWLAYIQELRNGRSSDDEATADSQHAAPPKKKRK
VGGSGGSGGSGGSGGSGGSGGSGGSLEHHHHHH
AsCas12a variant 3 amino acid sequence:
[SEQ ID NO: 43]
MTQFEGFTNLYQVSKTLRFELIPQGKTLKHIQEQGFIEEDKARNDHYKELKPIIDRIY
KTYADQCLQLVQLDWENLSAAIDSYRKEKTEETRNALIEEQATYRNAIHDYFIGRT
DNLTDAINKRHAEIYKGLFKAELFNGKVLKQLGTVTTTEHENALLRSFDKFTTYFSG
FYENRKNVFSAEDISTAIPHRIVQDNFPKFKENCHIFTRLITAVPSLREHFENVKKAIG
IFVSTSIEEVFSFPFYNQLLTQTQIDLYNQLLGGISREAGTEKIKGLNEVLNLAIQKND
ETAHIIASLPHRFIPLFKQILSDRNTLSFILEEFKSDEEVIQSFCKYKTLLRNENVLETA
EALFNELNSIDLTHIFISHKKLETISSALCDHWDTLRNALYERRISELTGKITKSAKEK
VQRSLKHEDINLQEIISAAGKELSEAFKQKTSEILSHAHAALDQPLPTTLKKQEEKEI
LKSQLDSLLGLYHLLDWFAVDESNEVDPEFSARLTGIKLEMEPSLSFYNKARNYAT
KKPYSVEKFKLNFQRPTLASGWDVNKEKNNGAILFVKNGLYYLGIMPKQKGRYKA
LSFEPTEKTSEGFDKMYYDYFPDAAKMIPKCSTQLKAVTAHFQTHTTPILLSNNFIEP
LEITKEIYDLNNPEKEPKKFQTAYAKKTGDQKGYREALCKWIDFTRDFLSKYTKTTS
IDLSSLRPSSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAARLGEK
MLNKKLKDQKTPIPDTLYQELYDYVNHRLSHDLSDEARALLPNVITKEVSHEIIKDR
RFTSDKFLFHVPITLNYQAANSPSKFNQRVNAYLKEHPETPIIGIDRGERNLIYITVID
STGKILEQRSLNTIQQFDYQKKLDNREKERVAARQAWSVVGTIKDLKQGYLSQVIH
EIVDLMIHYQAVVVLENLNFGFKSKRTGIAEKAVYQQFEKMLIDKLNCLVLKDYPA
EKVGGVLNPYQLTDQFTSFAKMGTQSGFLFYVPAPYTSKIDPLTGFVDPFVWKTIK
NHESRKHFLEGFDFLHYDVKTGDFILHFKMNRNLSFQRGLPGFMPAWDIVFEKNET
QFDAKGTPFIAGKRIVPVIENHRFTGRYRDLYPANELIALLEEKGIVFRDGSNILPKLL
ENDDSHAIDTMVALIRSVLQMRNSNAATGEDYINSPVRDLNGVCFDSRFQNPEWP
MDADANGAYHIALKGQLLLNHLKESKDLKLQNGISNQDWLAYIQELRNGRSSDDE
ATADSQHAAPPKKKRKVGGSGGSGGSGGSGGSGGSGGSGGSLEHHHHHH
AsCas12a variant 4 amino acid sequence:
[SEQ ID NO: 44]
MTQFEGFTNLYQVSKTLRFELIPQGKTLKHIQEQGFIEEDKARNDHYKELKPIIDRIY
KTYADQCLQLVQLDWENLSAAIDSYRKEKTEETRNALIEEQATYRNAIHDYFIGRT
DNLTDAINKRHAEIYKGLFKAELFNGKVLKQLGTVTTTEHENALLRSFDKFTTYFSG
FYENRKNVFSAEDISTAIPHRIVQDNFPKFKENCHIFTRLITAVPSLREHFENVKKAIG
IFVSTSIEEVFSFPFYNQLLTQTQIDLYNQLLGGISREAGTEKIKGLNEVLNLAIQKND
ETAHIIASLPHRFIPLFKQILSDRNTLSFILEEFKSDEEVIQSFCKYKTLLRNENVLETA
EALFNELNSIDLTHIFISHKKLETISSALCDHWDTLRNALYERRISELTGKITKSAKEK
VQRSLKHEDINLQEIISAAGKELSEAFKQKTSEILSHAHAALDQPLPTTLKKQEEKEI
LKSQLDSLLGLYHLLDWFAVDESNEVDPEFSARLTGIKLEMEPSLSFYNKARNYAT
KKPYSVEKFKLNFQRPTLASGWDVNKEKNNGAILFVKNGLYYLGIMPKQKGRYKA
LSFEPTEKTSEGFDKMYYDYFPDAAKMIPKCSTQLKAVTAHFQTHTTPILLSNNFIEP
LEITKEIYDLNNPEKEPKKFQTAYAKKTGDQKGYREALCKWIDFTRDFLSKYTKTTS
IDLSSLRPSSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAARLGEK
MLNKKLKDQKTPIPDTLYQELYDYVNHRLSHDLSDEARALLPNVITKEVSHEIIKDR
RFTSDKFLFHVPITLNYQAANSPSKFNQRVNAYLKEHPETPIIGIDRGERNLIYITVID
STGKILEQRSLNTIQQFDYQKKLDNREKERVAARQAWSVVGTIKDLKQGYLSQVIH
EIVDLMIHYQAVVVLENLNFGFKSKRTGIAEKAVYQQFEKMLIDKLNCLVLKDYPA
EKVGGVLNPYQLTDQFTSFAKMGTQSGFLFYVPAPYTSKIDPLTGFVDPFVWKTIK
NHESRKHFLEGFDFLHYDVKTGDFILHFKMNRNLSFQRGLPGFMPAWDIVFEKNET
QFDAKGTPFIAGKRIVPVIENHRFTGRYRDLYPANELIALLEEKGIVFRDGSNILPKLL
ENDDSHAIDTMVALIRSVLQMRNSNAATGEDYINSPVRDLNGVCFDSRFQNPEWP
MDADANGAYHIALKGQLLLNHLKESKDLKLQNGISNQDWLAYIQELRNGRSSDDE
ATADSQHAAPPKKKRKV
AsCas12a variant 4 nucleotide sequence:
[SEQ ID NO: 45]
ATGACCCAGTTTGAAGGTTTCACCAATCTGTATCAGGTTAGCAAAACCCTGCGTT
TTGAACTGATTCCGCAGGGTAAAACCCTGAAACATATTCAAGAACAGGGCTTCA
TCGAAGAGGATAAAGCACGTAACGATCACTACAAAGAACTGAAACCGATTATCG
ACCGCATCTATAAAACCTATGCAGATCAGTGTCTGCAGCTGGTTCAGCTGGATTG
GGAAAATCTGAGCGCAGCAATTGATAGTTATCGCAAAGAAAAAACCGAAGAAA
CCCGTAATGCACTGATTGAAGAACAGGCAACCTATCGTAATGCCATCCATGATTA
TTTCATTGGTCGTACCGATAATCTGACCGATGCAATTAACAAACGTCACGCCGAA
ATCTATAAAGGCCTGTTTAAAGCCGAACTGTTTAATGGCAAAGTTCTGAAACAGC
TGGGCACCGTTACCACCACCGAACATGAAAATGCACTGCTGCGTAGCTTTGATAA
ATTCACCACCTATTTCAGCGGCTTTTATGAGAATCGCAAAAACGTGTTTAGCGCA
GAAGATATTAGCACCGCAATTCCGCATCGTATTGTGCAGGATAATTTCCCGAAAT
TCAAAGAGAACTGCCACATTTTTACCCGTCTGATTACCGCAGTTCCGAGCCTGCG
TGAACATTTTGAAAACGTTAAAAAAGCCATCGGCATCTTTGTTAGCACCAGCATT
GAAGAAGTTTTTAGCTTCCCGTTTTACAATCAGCTGCTGACCCAGACCCAGATTG
ATCTGTATAACCAACTGCTGGGTGGTATTAGCCGTGAAGCAGGCACCGAAAAAA
TCAAAGGTCTGAATGAAGTGCTGAATCTGGCCATTCAGAAAAATGATGAAACCG
CACATATTATTGCAAGCCTGCCGCATCGTTTTATTCCGCTGTTCAAACAAATTCTG
AGCGATCGTAATACCCTGAGCTTTATTCTGGAAGAATTCAAATCCGATGAAGAG
GTGATTCAGAGCTTTTGCAAATACAAAACGCTGCTGCGCAATGAAAATGTTCTGG
AAACTGCCGAAGCACTGTTTAACGAACTGAATAGCATTGATCTGACCCACATCTT
TATCAGCCACAAAAAACTGGAAACCATTTCAAGCGCACTGTGTGATCATTGGGA
TACCCTGCGTAATGCCCTGTATGAACGTCGTATTAGCGAACTGACCGGTAAAATT
ACCAAAAGCGCGAAAGAAAAAGTTCAGCGCAGTCTGAAACATGAGGATATTAAT
CTGCAAGAGATTATTAGCGCAGCCGGTAAAGAACTGTCAGAAGCATTTAAACAG
AAAACCAGCGAAATTCTGTCACATGCACATGCAGCACTGGATCAGCCGCTGCCG
ACCACCCTGAAAAAACAAGAAGAAAAAGAAATCCTGAAAAGCCAGCTGGATAG
CCTGCTGGGTCTGTATCATCTGCTGGACTGGTTTGCAGTTGATGAAAGCAATGAA
GTTGATCCGGAATTTAGCGCACGTCTGACCGGCATTAAACTGGAAATGGAACCG
AGCCTGAGCTTTTATAACAAAGCCCGTAATTATGCCACCAAAAAACCGTATAGC
GTCGAAAAATTCAAACTGAACTTTCAGCGTCCGACCCTGGCAAGCGGTTGGGAT
GTTAATAAAGAAAAAAACAACGGTGCCATCCTGTTCGTGAAAAATGGCCTGTAT
TATCTGGGTATTATGCCGAAACAGAAAGGTCGTTATAAAGCGCTGAGCTTTGAAC
CGACGGAAAAAACCAGTGAAGGTTTTGATAAAATGTACTACGACTATTTTCCGG
ATGCAGCCAAAATGATTCCGAAATGTAGCACCCAGCTGAAAGCAGTTACCGCAC
ATTTTCAGACCCATACCACCCCGATTCTGCTGAGCAATAACTTTATTGAACCGCT
GGAAATCACCAAAGAGATCTACGATCTGAATAACCCGGAAAAAGAGCCGAAAA
AATTCCAGACCGCATATGCAAAAAAAACCGGTGATCAGAAAGGTTATCGTGAAG
CGCTGTGTAAATGGATTGATTTCACCCGTGATTTTCTGAGCAAATACACCAAAAC
CACCAGTATCGATCTGAGCAGCCTGCGTCCGAGCAGCCAGTATAAAGATCTGGG
CGAATATTATGCAGAACTGAATCCGCTGCTGTATCATATTAGCTTTCAGCGTATT
GCCGAGAAAGAAATCATGGACGCAGTTGAAACCGGTAAACTGTACCTGTTCCAG
ATCTACAATAAAGATTTTGCCAAAGGCCATCATGGCAAACCGAATCTGCATACCC
TGTATTGGACCGGTCTGTTTAGCCCTGAAAATCTGGCAAAAACCTCGATTAAACT
GAATGGTCAGGCGGAACTGTTTTATCGTCCGAAAAGCCGTATGAAACGTATGGC
AGCTCGTCTGGGTGAAAAAATGCTGAACAAAAAACTGAAAGACCAGAAAACCCC
GATCCCGGATACACTGTATCAAGAACTGTATGATTATGTGAACCATCGTCTGAGC
CATGATCTGAGTGATGAAGCACGTGCCCTGCTGCCGAATGTTATTACCAAAGAA
GTTAGCCACGAGATCATTAAAGATCGTCGTTTTACCAGCGACAAATTCCTGTTTC
ATGTGCCGATTACCCTGAATTATCAGGCAGCAAATAGCCCGAGCAAATTTAACC
AGCGTGTTAATGCATATCTGAAAGAACATCCAGAAACGCCGATTATTGGTATTGA
TCGTGGTGAACGTAACCTGATTTATATCACCGTTATTGATAGCACCGGCAAAATC
CTGGAACAGCGTAGCCTGAATACCATTCAGCAGTTTGATTACCAGAAAAAACTG
GATAATCGCGAGAAAGAACGTGTTGCAGCACGTCAGGCATGGTCAGTTGTTGGT
ACAATTAAAGACCTGAAACAGGGTTATCTGAGCCAGGTTATTCATGAAATTGTGG
ATCTGATGATTCACTATCAGGCCGTTGTTGTGCTGGAAAACCTGAATTTTGGCTTT
AAAAGCAAACGTACCGGCATTGCAGAAAAAGCAGTTTATCAGCAGTTCGAGAAA
ATGCTGATTGACAAACTGAATTGCCTGGTGCTGAAAGATTATCCGGCTGAAAAA
GTTGGTGGTGTTCTGAATCCGTATCAGCTGACCGATCAGTTTACCAGCTTTGCAA
AAATGGGCACCCAGAGCGGATTTCTGTTTTATGTTCCGGCACCGTATACGAGCAA
AATTGATCCGCTGACCGGTTTTGTTGATCCGTTTGTTTGGAAAACCATCAAAAAC
CATGAAAGCCGCAAACATTTTCTGGAAGGTTTCGATTTTCTGCATTACGACGTTA
AAACGGGTGATTTCATCCTGCACTTTAAAATGAATCGCAATCTGAGTTTTCAGCG
TGGCCTGCCTGGTTTTATGCCTGCATGGGATATTGTGTTTGAGAAAAACGAAACA
CAGTTCGATGCAAAAGGCACCCCGTTTATTGCAGGTAAACGTATTGTTCCGGTGA
TTGAAAATCATCGTTTCACCGGTCGTTATCGCGATCTGTATCCGGCAAATGAACT
GATCGCACTGCTGGAAGAGAAAGGTATTGTTTTTCGTGATGGCTCAAACATTCTG
CCGAAACTGCTGGAAAATGATGATAGCCATGCAATTGATACCATGGTTGCACTG
ATTCGTAGCGTTCTGCAGATGCGTAATAGCAATGCAGCAACCGGTGAAGATTAC
ATTAATAGTCCGGTTCGTGATCTGAATGGTGTTTGTTTTGATAGCCGTTTTCAGAA
TCCGGAATGGCCGATGGATGCAGATGCAAATGGTGCATATCATATTGCACTGAA
AGGACAGCTGCTGCTGAACCACCTGAAAGAAAGCAAAGATCTGAAACTGCAAAA
CGGCATTAGCAATCAGGATTGGCTGGCATATATCCAAGAACTGCGTAACGGTCG
TAGCAGTGATGATGAAGCAACCGCAGATAGCCAGCATGCAGCACCGCCTAAAAA
GAAACGTAAAGTT
AsCas12a variant 5 amino acid sequence:
[SEQ ID NO: 46]
MTQFEGFTNLYQVSKTLRFELIPQGKTLKHIQEQGFIEEDKARNDHYKELKPIIDRIY
KTYADQCLQLVQLDWENLSAAIDSYRKEKTEETRNALIEEQATYRNAIHDYFIGRT
DNLTDAINKRHAEIYKGLFKAELFNGKVLKQLGTVTTTEHENALLRSFDKFTTYFSG
FYENRKNVFSAEDISTAIPHRIVQDNFPKFKENCHIFTRLITAVPSLREHFENVKKAIG
IFVSTSIEEVFSFPFYNQLLTQTQIDLYNQLLGGISREAGTEKIKGLNEVLNLAIQKND
ETAHIIASLPHRFIPLFKQILSDRNTLSFILEEFKSDEEVIQSFCKYKTLLRNENVLETA
EALFNELNSIDLTHIFISHKKLETISSALCDHWDTLRNALYERRISELTGKITKSAKEK
VQRSLKHEDINLQEIISAAGKELSEAFKQKTSEILSHAHAALDQPLPTTLKKQEEKEI
LKSQLDSLLGLYHLLDWFAVDESNEVDPEFSARLTGIKLEMEPSLSFYNKARNYAT
KKPYSVEKFKLNFQRPTLASGWDVNKEKNNGAILFVKNGLYYLGIMPKQKGRYKA
LSFEPTEKTSEGFDKMYYDYFPDAAKMIPKCSTQLKAVTAHFQTHTTPILLSNNFIEP
LEITKEIYDLNNPEKEPKKFQTAYAKKTGDQKGYREALCKWIDFTRDFLSKYTKTTS
IDLSSLRPSSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAHRLGEK
MLNKKLKDQKTPIPDTLYQELYDYVNHRLSHDLSDEARALLPNVITKEVSHEIIKDR
RFTSDKFLFHVPITLNYQAANSPSKFNQRVNAYLKEHPETPIIGIDRGERNLIYITVID
STGKILEQRSLNTIQQFDYQKKLDNREKERVAARQAWSVVGTIKDLKQGYLSQVIH
EIVDLMIHYQAVVVLENLNFGFKSKRTGIAEKAVYQQFEKMLIDKLNCLVLKDYPA
EKVGGVLNPYQLTDQFTSFAKMGTQSGFLFYVPAPYTSKIDPLTGFVDPFVWKTIK
NHESRKHFLEGFDFLHYDVKTGDFILHFKMNRNLSFQRGLPGFMPAWDIVFEKNET
QFDAKGTPFIAGKRIVPVIENHRFTGRYRDLYPANELIALLEEKGIVFRDGSNILPKLL
ENDDSHAIDTMVALIRSVLQMRNSNAATGEDYINSPVRDLNGVCFDSRFQNPEWP
MDADANGAYHIALKGQLLLNHLKESKDLKLQNGISNQDWLAYIQELRNGRSSDDE
ATADSQHAAPPKKKRKV
AsCas12a variant 6 amino acid sequence:
[SEQ ID NO: 47]
MTQFEGFTNLYQVSKTLRFELIPQGKTLKHIQEQGFIEEDKARNDHYKELKPIIDRIY
KTYADQCLQLVQLDWENLSAAIDSYRKEKTEETRNALIEEQATYRNAIHDYFIGRT
DNLTDAINKRHAEIYKGLFKAELFNGKVLKQLGTVTTTEHENALLRSFDKFTTYFSG
FYENRKNVFSAEDISTAIPHRIVQDNFPKFKENCHIFTRLITAVPSLREHFENVKKAIG
IFVSTSIEEVFSFPFYNQLLTQTQIDLYNQLLGGISREAGTEKIKGLNEVLNLAIQKND
ETAHIIASLPHRFIPLFKQILSDRNTLSFILEEFKSDEEVIQSFCKYKTLLRNENVLETA
EALFNELNSIDLTHIFISHKKLETISSALCDHWDTLRNALYERRISELTGKITKSAKEK
VQRSLKHEDINLQEIISAAGKELSEAFKQKTSEILSHAHAALDQPLPTTLKKQEEKEI
LKSQLDSLLGLYHLLDWFAVDESNEVDPEFSARLTGIKLEMEPSLSFYNKARNYAT
KKPYSVEKFKLNFQRPTLASGWDVNKEKNNGAILFVKNGLYYLGIMPKQKGRYKA
LSFEPTEKTSEGFDKMYYDYFPDAAKMIPKCSTQLKAVTAHFQTHTTPILLSNNFIEP
LEITKEIYDLNNPEKEPKKFQTAYAKKTGDQKGYREALCKWIDFTRDFLSKYTKTTS
IDLSSLRPSSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAHRLGEK
MLNKKLKDQKTPIPDTLYQELYDYVNHRLSHDLSDEARALLPNVITKEVSHEIIKDR
RFTSDKFLFHVPITLNYQAANSPSKFNQRVNAYLKEHPETPIIGIDRGERNLIYITVID
STGKILEQRSLNTIQQFDYQKKLDNREKERVAARQAWSVVGTIKDLKQGYLSQVIH
EIVDLMIHYQAVVVLENLNFGFKSKRTGIAEKAVYQQFEKMLIDKLNCLVLKDYPA
EKVGGVLNPYQLTDQFTSFAKMGTQSGFLFYVPAPYTSKIDPLTGFVDPFVWKTIK
NHESRKHFLEGFDFLHYDVKTGDFILHFKMNRNLSFQRGLPGFMPAWDIVFEKNET
QFDAKGTPFIAGKRIVPVIENHRFTGRYRDLYPANELIALLEEKGIVFRDGSNILPKLL
ENDDSHAIDTMVALIRSVLQMRNSNAATGEDYINSPVRDLNGVCFDSRFQNPEWP
MDADANGAYHIALKGQLLLNHLKESKDLKLQNGISNQDWLAYIQELRNGRSSDDE
ATADSQHAAPPKKKRKVGGSGGSGGSGGSGGSGGSGGSGGSLEHHHHHH
AsCas12a variant 7 amino acid sequence:
[SEQ ID NO: 48]
MGRDPGKPIPNPLLGLDSTAPKKKRKVGIHGVPAATQFEGFTNLYQVSKTLRFELIP
QGKTLKHIQEQGFIEEDKARNDHYKELKPIIDRIYKTYADQCLQLVQLDWENLSAAI
DSYRKEKTEETRNALIEEQATYRNAIHDYFIGRTDNLTDAINKRHAEIYKGLFKAEL
FNGKVLKQLGTVTTTEHENALLRSFDKFTTYFSGFYENRKNVFSAEDISTAIPHRIVQ
DNFPKFKENCHIFTRLITAVPSLREHFENVKKAIGIFVSTSIEEVFSFPFYNQLLTQTQI
DLYNQLLGGISREAGTEKIKGLNEVLNLAIQKNDETAHIIASLPHRFIPLFKQILSDRN
TLSFILEEFKSDEEVIQSFCKYKTLLRNENVLETAEALFNELNSIDLTHIFISHKKLETI
SSALCDHWDTLRNALYERRISELTGKITKSAKEKVQRSLKHEDINLQEIISAAGKELS
EAFKQKTSEILSHAHAALDQPLPTTLKKQEEKEILKSQLDSLLGLYHLLDWFAVDES
NEVDPEFSARLTGIKLEMEPSLSFYNKARNYATKKPYSVEKFKLNFQMPTLASGWD
VNKEKNNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEKEPKKFQTA
YAKKTGDQKGYREALCKWIDFTRDFLSKYTKTTSIDLSSLRPSSQYKDLGEYYAEL
NPLLYHISFQRIAEKEIMDAVETGKLYLFQIYNKDFAKGHHGKPNLHTLYWTGLFSP
ENLAKTSIKLNGQAELFYRPKSRMKRMAHRLGEKMLNKKLKDQKTPIPDTLYQEL
YDYVNHRLSHDLSDEARALLPNVITKEVSHEIIKDRRFTSDKFFFHVPITLNYQAANS
PSKFNQRVNAYLKEHPETPIIGIDRGERNLIYITVIDSTGKILEQRSLNTIQQFDYQKK
LDNREKERVAARQAWSVVGTIKDLKQGYLSQVIHEIVDLMIHYQAVVVLENLNFG
FKSKRTGIAEKAVYQQFEKMLIDKLNCLVLKDYPAEKVGGVLNPYQLTDQFTSFAK
MGTQSGFLFYVPAPYTSKIDPLTGFVDPFVWKTIKNHESRKHFLEGFDFLHYDVKT
GDFILHFKMNRNLSFQRGLPGFMPAWDIVFEKNETQFDAKGTPFIAGKRIVPVIENH
RFTGRYRDLYPANELIALLEEKGIVFRDGSNILPKLLENDDSHAIDTMVALIRSVLQ
MRNSNAATGEDYINSPVRDLNGVCFDSRFQNPEWPMDADANGAYHIALKGQLLLN
HLKESKDLKLQNGISNQDWLAYIQELRNPKKKRKVKLAAALEHHHHHH
AsCas12a variant 8 amino acid sequence:
[SEQ ID NO: 1348]
MTQFEGFTNLYQVSKTLRFELIPQGKTLKHIQEQGFIEEDKARNDHYKELKPIIDRIYK
TYADQCLQLVQLDWENLSAAIDSYRKEKTEETRNALIEEQATYRNAIHDYFIGRTDN
LTDAINKRHAEIYKGLFKAELFNGKVLKQLGTVTTTEHENALLRSFDKFTTYFSGFYE
NRKNVFSAEDISTAIPHRIVQDNFPKFKENCHIFTRLITAVPSLREHFENVKKAIGIFVS
TSIEEVFSFPFYNQLLTQTQIDLYNQLLGGISREAGTEKIKGLNEVLNLAIQKNDETAH
IIASLPHRFIPLFKQILSDRNTLSFILEEFKSDEEVIQSFCKYKTLLRNENVLETAEALFN
ELNSIDLTHIFISHKKLETISSALCDHWDTLRNALYERRISELTGKITKSAKEKVQRSL
KHEDINLQEIISAAGKELSEAFKQKTSEILSHAHAALDQPLPTTLKKQEEKEILKSQLD
SLLGLYHLLDWFAVDESNEVDPEFSARLTGIKLEMEPSLSFYNKARNYATKKPYSVE
KFKLNFQRPTLASGWDVNKEKNNGAILFVKNGLYYLGIMPKQKGRYKALSFEPTEK
TSEGFDKMYYDYFPDAAKMIPKCSTQLKAVTAHFQTHTTPILLSNNFIEPLEITKEIYD
LNNPEKEPKKFQTAYAKKTGDQKGYREALCKWIDFTRDFLSKYTKTTSIDLSSLRPSS
QYKDLGEYYAELNPLLYHISFQRIAEKEIMDAVETGKLYLFQIYNKDFAKGHHGKPN
LHTLYWTGLFSPENLAKTSIKLNGQAELFYRPKSRMKRMAARLGEKMLNKKLKDQ
KTPIPDTLYQELYDYVNHRLSHDLSDEARALLPNVITKEVSHEIIKDRRFTSDKFLFHV
PITLNYQAANSPSKFNQRVNAYLKEHPETPIIGIDRGERNLIYITVIDSTGKILEQRSLN
TIQQFDYQKKLDNREKERVAARQAWSVVGTIKDLKQGYLSQVIHEIVDLMIHYQAV
VVLENLNFGFKSKRTGIAEKAVYQQFEKMLIDKLNCLVLKDYPAEKVGGVLNPYQL
TDQFTSFAKMGTQSGFLFYVPAPYTSKIDPLTGFVDPFVWKTIKNHESRKHFLEGFDF
LHYDVKTGDFILHFKMNRNLSFQRGLPGFMPAWDIVFEKNETQFDAKGTPFIAGKRI
VPVIENHRFTGRYRDLYPANELIALLEEKGIVFRDGSNILPKLLENDDSHAIDTMVALI
RSVLQMRNSNAATGEDYINSPVRDLNGVCFDSRFQNPEWPMDADANGAYHIALKG
QLLLNHLKESKDLKLQNGISNQDWLAYIQELRNGGSPAAKRVKLDGGSPAAKRVKL
D
Exemplary AsCas12a wild-type amino acid sequence:
[SEQ ID NO: 49]
MTQFEGFTNLYQVSKTLRFELIPQGKTLKHIQEQGFIEEDKARNDHYKELKPIIDRIY
KTYADQCLQLVQLDWENLSAAIDSYRKEKTEETRNALIEEQATYRNAIHDYFIGRT
DNLTDAINKRHAEIYKGLFKAELFNGKVLKQLGTVTTTEHENALLRSFDKFTTYFSG
FYENRKNVFSAEDISTAIPHRIVQDNFPKFKENCHIFTRLITAVPSLREHFENVKKAIG
IFVSTSIEEVFSFPFYNQLLTQTQIDLYNQLLGGISREAGTEKIKGLNEVLNLAIQKND
ETAHIIASLPHRFIPLFKQILSDRNTLSFILEEFKSDEEVIQSFCKYKTLLRNENVLETA
EALFNELNSIDLTHIFISHKKLETISSALCDHWDTLRNALYERRISELTGKITKSAKEK
VQRSLKHEDINLQEIISAAGKELSEAFKQKTSEILSHAHAALDQPLPTTLKKQEEKEI
LKSQLDSLLGLYHLLDWFAVDESNEVDPEFSARLTGIKLEMEPSLSFYNKARNYAT
KKPYSVEKFKLNFQMPTLASGWDVNKEKNNGAILFVKNGLYYLGIMPKQKGRYK
ALSFEPTEKTSEGFDKMYYDYFPDAAKMIPKCSTQLKAVTAHFQTHTTPILLSNNFI
EPLEITKEIYDLNNPEKEPKKFQTAYAKKTGDQKGYREALCKWIDFTRDFLSKYTKT
TSIDLSSLRPSSQYKDLGEYYAELNPLLYHISFQRIAEKEIMDAVETGKLYLFQIYNK
DFAKGHHGKPNLHTLYWTGLFSPENLAKTSIKLNGQAELFYRPKSRMKRMAHRLG
EKMLNKKLKDQKTPIPDTLYQELYDYVNHRLSHDLSDEARALLPNVITKEVSHEIIK
DRRFTSDKFFFHVPITLNYQAANSPSKFNQRVNAYLKEHPETPIIGIDRGERNLIYITV
IDSTGKILEQRSLNTIQQFDYQKKLDNREKERVAARQAWSVVGTIKDLKQGYLSQV
IHEIVDLMIHYQAVVVLENLNFGFKSKRTGIAEKAVYQQFEKMLIDKLNCLVLKDY
PAEKVGGVLNPYQLTDQFTSFAKMGTQSGFLFYVPAPYTSKIDPLTGFVDPFVWKTI
KNHESRKHFLEGFDFLHYDVKTGDFILHFKMNRNLSFQRGLPGFMPAWDIVFEKNE
TQFDAKGTPFIAGKRIVPVIENHRFTGRYRDLYPANELIALLEEKGIVFRDGSNILPKL
LENDDSHAIDTMVALIRSVLQMRNSNAATGEDYINSPVRDLNGVCFDSRFQNPEWP
MDADANGAYHIALKGQLLLNHLKESKDLKLQNGISNQDWLAYIQELRN
AsCas12a-MHFRR amino acid sequence:
[SEQ ID NO: 50]
MTQFEGFTNLYQVSKTLRFELIPQGKTLKHIQEQGFIEEDKARNDHYKELKPIIDRIYK
TYADQCLQLVQLDWENLSAAIDSYRKEKTEETRNALIEEQATYRNAIHDYFIGRTDN
LTDAINKRHAEIYKGLFKAELFNGKVLKQLGTVTTTEHENALLRSFDKFTTYFSGFYE
NRKNVFSAEDISTAIPHRIVQDNFPKFKENCHIFTRLITAVPSLREHFENVKKAIGIFVS
TSIEEVFSFPFYNQLLTQTQIDLYNQLLGGISREAGTEKIKGLNEVLNLAIQKNDETAH
IIASLPHRFIPLFKQILSDRNTLSFILEEFKSDEEVIQSFCKYKTLLRNENVLETAEALFN
ELNSIDLTHIFISHKKLETISSALCDHWDTLRNALYERRISELTGKITKSAKEKVQRSL
KHEDINLQEIISAAGKELSEAFKQKTSEILSHAHAALDQPLPTTLKKQEEKEILKSQLD
SLLGLYHLLDWFAVDESNEVDPEFSARLTGIKLEMEPSLSFYNKARNYATKKPYSVE
KFKLNFQRPTLARGWDVNKEKNNGAILFVKNGLYYLGIMPKQKGRYKALSFEPTEK
TSEGFDKMYYDYFPDAAKMIPRCSTQLKAVTAHFQTHTTPILLSNNFIEPLEITKEIYD
LNNPEKEPKKFQTAYAKKTGDQKGYREALCKWIDFTRDFLSKYTKTTSIDLSSLRPSS
QYKDLGEYYAELNPLLYHISFQRIAEKEIMDAVETGKLYLFQIYNKDFAKGHHGKPN
LHTLYWTGLFSPENLAKTSIKLNGQAELFYRPKSRMKRMAARLGEKMLNKKLKDQ
KTPIPDTLYQELYDYVNHRLSHDLSDEARALLPNVITKEVSHEIIKDRRFTSDKFLFHV
PITLNYQAANSPSKFNQRVNAYLKEHPETPIIGIDRGERNLIYITVIDSTGKILEQRSLN
TIQQFDYQKKLDNREKERVAARQAWSVVGTIKDLKQGYLSQVIHEIVDLMIHYQAV
VVLENLNFGFKSKRTGIAEKAVYQQFEKMLIDKLNCLVLKDYPAEKVGGVLNPYQL
TDQFTSFAKMGTQSGFLFYVPAPYTSKIDPLTGFVDPFVWKTIKNHESRKHFLEGFDF
LHYDVKTGDFILHFKMNRNLSFQRGLPGFMPAWDIVFEKNETQFDAKGTPFIAGKRI
VPVIENHRFTGRYRDLYPANELIALLEEKGIVFRDGSNILPKLLENDDSHAIDTMVALI
RSVLQMRNSNAATGEDYINSPVRDLNGVCFDSRFQNPEWPMDADANGAYHIALKG
QLLLNHLKESKDLKLQNGISNQDWLAYIQELRNGGSPAAKRVKLDGGSPAAKRVKL
D
AsCas12a-MHFRR amino acid sequence:
[SEQ ID NO: 51]
MTQFEGFTNLYQVSKTLRFELIPQGKTLKHIQEQGFIEEDKARNDHYKELKPIIDRIYK
TYADQCLQLVQLDWENLSAAIDSYRKEKTEETRNALIEEQATYRNAIHDYFIGRTDN
LTDAINKRHAEIYKGLFKAELFNGKVLKQLGTVTTTEHENALLRSFDKFTTYFSGFYE
NRKNVFSAEDISTAIPHRIVQDNFPKFKENCHIFTRLITAVPSLREHFENVKKAIGIFVS
TSIEEVFSFPFYNQLLTQTQIDLYNQLLGGISREAGTEKIKGLNEVLNLAIQKNDETAH
IIASLPHRFIPLFKQILSDRNTLSFILEEFKSDEEVIQSFCKYKTLLRNENVLETAEALFN
ELNSIDLTHIFISHKKLETISSALCDHWDTLRNALYERRISELTGKITKSAKEKVQRSL
KHEDINLQEIISAAGKELSEAFKQKTSEILSHAHAALDQPLPTTLKKQEEKEILKSQLD
SLLGLYHLLDWFAVDESNEVDPEFSARLTGIKLEMEPSLSFYNKARNYATKKPYSVE
KFKLNFQRPTLARGWDVNKEKNNGAILFVKNGLYYLGIMPKQKGRYKALSFEPTEK
TSEGFDKMYYDYFPDAAKMIPRCSTQLKAVTAHFQTHTTPILLSNNFIEPLEITKEIYD
LNNPEKEPKKFQTAYAKKTGDQKGYREALCKWIDFTRDFLSKYTKTTSIDLSSLRPSS
QYKDLGEYYAELNPLLYHISFQRIAEKEIMDAVETGKLYLFQIYNKDFAKGHHGKPN
LHTLYWTGLFSPENLAKTSIKLNGQAELFYRPKSRMKRMAARLGEKMLNKKLKDQ
KTPIPDTLYQELYDYVNHRLSHDLSDEARALLPNVITKEVSHEIIKDRRFTSDKFLFHV
PITLNYQAANSPSKFNQRVNAYLKEHPETPIIGIDRGERNLIYITVIDSTGKILEQRSLN
TIQQFDYQKKLDNREKERVAARQAWSVVGTIKDLKQGYLSQVIHEIVDLMIHYQAV
VVLENLNFGFKSKRTGIAEKAVYQQFEKMLIDKLNCLVLKDYPAEKVGGVLNPYQL
TDQFTSFAKMGTQSGFLFYVPAPYTSKIDPLTGFVDPFVWKTIKNHESRKHFLEGFDF
LHYDVKTGDFILHFKMNRNLSFQRGLPGFMPAWDIVFEKNETQFDAKGTPFIAGKRI
VPVIENHRFTGRYRDLYPANELIALLEEKGIVFRDGSNILPKLLENDDSHAIDTMVALI
RSVLQMRNSNAATGEDYINSPVRDLNGVCFDSRFQNPEWPMDADANGAYHIALKG
QLLLNHLKESKDLKLQNGISNQDWLAYIQELRNGRSSDDEATADSQHAAPPKKKRK
V
AsCas12a-MHFRVR amino acid sequence:
[SEQ ID NO: 52]
MTQFEGFTNLYQVSKTLRFELIPQGKTLKHIQEQGFIEEDKARNDHYKELKPIIDRIYK
TYADQCLQLVQLDWENLSAAIDSYRKEKTEETRNALIEEQATYRNAIHDYFIGRTDN
LTDAINKRHAEIYKGLFKAELFNGKVLKQLGTVTTTEHENALLRSFDKFTTYFSGFYE
NRKNVFSAEDISTAIPHRIVQDNFPKFKENCHIFTRLITAVPSLREHFENVKKAIGIFVS
TSIEEVFSFPFYNQLLTQTQIDLYNQLLGGISREAGTEKIKGLNEVLNLAIQKNDETAH
IIASLPHRFIPLFKQILSDRNTLSFILEEFKSDEEVIQSFCKYKTLLRNENVLETAEALFN
ELNSIDLTHIFISHKKLETISSALCDHWDTLRNALYERRISELTGKITKSAKEKVQRSL
KHEDINLQEIISAAGKELSEAFKQKTSEILSHAHAALDQPLPTTLKKQEEKEILKSQLD
SLLGLYHLLDWFAVDESNEVDPEFSARLTGIKLEMEPSLSFYNKARNYATKKPYSVE
KFKLNFQRPTLARGWDVNVEKNRGAILFVKNGLYYLGIMPKQKGRYKALSFEPTEK
TSEGFDKMYYDYFPDAAKMIPKCSTQLKAVTAHFQTHTTPILLSNNFIEPLEITKEIYD
LNNPEKEPKKFQTAYAKKTGDQKGYREALCKWIDFTRDFLSKYTKTTSIDLSSLRPSS
QYKDLGEYYAELNPLLYHISFQRIAEKEIMDAVETGKLYLFQIYNKDFAKGHHGKPN
LHTLYWTGLFSPENLAKTSIKLNGQAELFYRPKSRMKRMAARLGEKMLNKKLKDQ
KTPIPDTLYQELYDYVNHRLSHDLSDEARALLPNVITKEVSHEIIKDRRFTSDKFLFHV
PITLNYQAANSPSKFNQRVNAYLKEHPETPIIGIDRGERNLIYITVIDSTGKILEQRSLN
TIQQFDYQKKLDNREKERVAARQAWSVVGTIKDLKQGYLSQVIHEIVDLMIHYQAV
VVLENLNFGFKSKRTGIAEKAVYQQFEKMLIDKLNCLVLKDYPAEKVGGVLNPYQL
TDQFTSFAKMGTQSGFLFYVPAPYTSKIDPLTGFVDPFVWKTIKNHESRKHFLEGFDF
LHYDVKTGDFILHFKMNRNLSFQRGLPGFMPAWDIVFEKNETQFDAKGTPFIAGKRI
VPVIENHRFTGRYRDLYPANELIALLEEKGIVFRDGSNILPKLLENDDSHAIDTMVALI
RSVLQMRNSNAATGEDYINSPVRDLNGVCFDSRFQNPEWPMDADANGAYHIALKG
QLLLNHLKESKDLKLQNGISNQDWLAYIQELRNGGSPAAKRVKLDGGSPAAKRVKL
D
AsCas12a-MHFRVR amino acid sequence:
[SEQ ID NO: 53]
MTQFEGFTNLYQVSKTLRFELIPQGKTLKHIQEQGFIEEDKARNDHYKELKPIIDRIY
KTYADQCLQLVQLDWENLSAAIDSYRKEKTEETRNALIEEQATYRNAIHDYFIGRT
DNLTDAINKRHAEIYKGLFKAELFNGKVLKQLGTVTTTEHENALLRSFDKFTTYFSG
FYENRKNVFSAEDISTAIPHRIVQDNFPKFKENCHIFTRLITAVPSLREHFENVKKAIG
IFVSTSIEEVFSFPFYNQLLTQTQIDLYNQLLGGISREAGTEKIKGLNEVLNLAIQKND
ETAHIIASLPHRFIPLFKQILSDRNTLSFILEEFKSDEEVIQSFCKYKTLLRNENVLETA
EALFNELNSIDLTHIFISHKKLETISSALCDHWDTLRNALYERRISELTGKITKSAKEK
VQRSLKHEDINLQEIISAAGKELSEAFKQKTSEILSHAHAALDQPLPTTLKKQEEKEI
LKSQLDSLLGLYHLLDWFAVDESNEVDPEFSARLTGIKLEMEPSLSFYNKARNYAT
KKPYSVEKFKLNFQRPTLARGWDVNVEKNRGAILFVKNGLYYLGIMPKQKGRYK
ALSFEPTEKTSEGFDKMYYDYFPDAAKMIPKCSTQLKAVTAHFQTHTTPILLSNNFI
EPLEITKEIYDLNNPEKEPKKFQTAYAKKTGDQKGYREALCKWIDFTRDFLSKYTKT
TSIDLSSLRPSSQYKDLGEYYAELNPLLYHISFQRIAEKEIMDAVETGKLYLFQIYNK
DFAKGHHGKPNLHTLYWTGLFSPENLAKTSIKLNGQAELFYRPKSRMKRMAARLG
EKMLNKKLKDQKTPIPDTLYQELYDYVNHRLSHDLSDEARALLPNVITKEVSHEIIK
DRRFTSDKFLFHVPITLNYQAANSPSKFNQRVNAYLKEHPETPIIGIDRGERNLIYITV
IDSTGKILEQRSLNTIQQFDYQKKLDNREKERVAARQAWSVVGTIKDLKQGYLSQV
IHEIVDLMIHYQAVVVLENLNFGFKSKRTGIAEKAVYQQFEKMLIDKLNCLVLKDY
PAEKVGGVLNPYQLTDQFTSFAKMGTQSGFLFYVPAPYTSKIDPLTGFVDPFVWKTI
KNHESRKHFLEGFDFLHYDVKTGDFILHFKMNRNLSFQRGLPGFMPAWDIVFEKNE
TQFDAKGTPFIAGKRIVPVIENHRFTGRYRDLYPANELIALLEEKGIVFRDGSNILPKL
LENDDSHAIDTMVALIRSVLQMRNSNAATGEDYINSPVRDLNGVCFDSRFQNPEWP
MDADANGAYHIALKGQLLLNHLKESKDLKLQNGISNQDWLAYIQELRNGRSSDDE
ATADSQHAAPPKKKRKV
AsCas12a-MHF amino acid sequence:
[SEQ ID NO: 54]
MTQFEGFTNLYQVSKTLRFELIPQGKTLKHIQEQGFIEEDKARNDHYKELKPIIDRIYK
TYADQCLQLVQLDWENLSAAIDSYRKEKTEETRNALIEEQATYRNAIHDYFIGRTDN
LTDAINKRHAEIYKGLFKAELFNGKVLKQLGTVTTTEHENALLRSFDKFTTYFSGFYE
NRKNVFSAEDISTAIPHRIVQDNFPKFKENCHIFTRLITAVPSLREHFENVKKAIGIFVS
TSIEEVFSFPFYNQLLTQTQIDLYNQLLGGISREAGTEKIKGLNEVLNLAIQKNDETAH
IIASLPHRFIPLFKQILSDRNTLSFILEEFKSDEEVIQSFCKYKTLLRNENVLETAEALFN
ELNSIDLTHIFISHKKLETISSALCDHWDTLRNALYERRISELTGKITKSAKEKVQRSL
KHEDINLQEIISAAGKELSEAFKQKTSEILSHAHAALDQPLPTTLKKQEEKEILKSQLD
SLLGLYHLLDWFAVDESNEVDPEFSARLTGIKLEMEPSLSFYNKARNYATKKPYSVE
KFKLNFQRPTLASGWDVNKEKNNGAILFVKNGLYYLGIMPKQKGRYKALSFEPTEK
TSEGFDKMYYDYFPDAAKMIPKCSTQLKAVTAHFQTHTTPILLSNNFIEPLEITKEIYD
LNNPEKEPKKFQTAYAKKTGDQKGYREALCKWIDFTRDFLSKYTKTTSIDLSSLRPSS
QYKDLGEYYAELNPLLYHISFQRIAEKEIMDAVETGKLYLFQIYNKDFAKGHHGKPN
LHTLYWTGLFSPENLAKTSIKLNGQAELFYRPKSRMKRMAARLGEKMLNKKLKDQ
KTPIPDTLYQELYDYVNHRLSHDLSDEARALLPNVITKEVSHEIIKDRRFTSDKFLFHV
PITLNYQAANSPSKFNQRVNAYLKEHPETPIIGIDRGERNLIYITVIDSTGKILEQRSLN
TIQQFDYQKKLDNREKERVAARQAWSVVGTIKDLKQGYLSQVIHEIVDLMIHYQAV
VVLENLNFGFKSKRTGIAEKAVYQQFEKMLIDKLNCLVLKDYPAEKVGGVLNPYQL
TDQFTSFAKMGTQSGFLFYVPAPYTSKIDPLTGFVDPFVWKTIKNHESRKHFLEGFDF
LHYDVKTGDFILHFKMNRNLSFQRGLPGFMPAWDIVFEKNETQFDAKGTPFIAGKRI
VPVIENHRFTGRYRDLYPANELIALLEEKGIVFRDGSNILPKLLENDDSHAIDTMVALI
RSVLQMRNSNAATGEDYINSPVRDLNGVCFDSRFQNPEWPMDADANGAYHIALKG
QLLLNHLKESKDLKLQNGISNQDWLAYIQELRNGRSSDDEATADSQHAAPPKKKRK
V

In some embodiments, an RNA-guided nuclease has at least 80%, at least 85%, at least 86%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity relative to a wild-type RNA-guided nuclease and/or an RNA-guided nuclease disclosed herein (e.g., an RNA-guided nuclease comprising an amino acid sequence selected from the group consisting of SEQ ID NOs: 40-44 and 46-54). In some embodiments, an RNA-guided nuclease has 1, 2, 3, 4, 5, 6, 7, 8, 9, 10, 11, 12, 13, 14, 15, 16, 17, 18, 19, or 20 mutations relative to a wild-type RNA-guided nuclease and/or an RNA-guided nuclease disclosed herein (e.g., an RNA-guided nuclease comprising an amino acid sequence selected from the group consisting of SEQ ID NOs: 40-44 and 46-54). In some embodiments, an RNA-guided nuclease has less than 2, 3, 4, 5, 6, 7, 8, 9, 10, 11, 12, 13, 14, 15, 16, 17, 18, 19, or 20 mutations relative to a wild-type RNA-guided nuclease and/or an RNA-guided nuclease disclosed herein (e.g., an RNA-guided nuclease comprising an amino acid sequence selected from the group consisting of SEQ ID NOs: 40-44 and 46-54).

### 5.4 Nucleic Acids Encoding RNA-Guided Nucleases

Nucleic acids encoding RNA-guided nucleases, e.g., Cas9, Cas12a or functional fragments thereof, are provided herein. Exemplary nucleic acids encoding RNA-guided nucleases have been described previously (see, e.g., Cong 2013; Wang 2013; Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, the nucleic acid encoding the RNA-guided nuclease is an RNA. In certain embodiments, the nucleic acid encoding the RNA-guided nuclease is an mRNA. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in Cotta-Ramusino.

In addition, or alternatively, a nucleic acid encoding an RNA-guided nuclease can comprise a nucleic acid encoding a nuclear localization sequence (NLS). Nuclear localization sequences are known in the art and examples include but are not limited to the NLS sequences fused to the RNA-guided nuclease sequence as indicated in SEQ ID NO: 40, presented herein.

Some non-limiting exemplary RNA sequences encoding the RNA-guided nuclease are as follows:

5.4.1 Cas9 mRNAs

SpCas9 RNA ORF #1
[SEQ ID NO: 1349]
AUGGCCCCCAAGAAGAAGCGGAAGGUGGGCAUCCACGGCGUGCCCGCCGCCGA
CAAGAAGUACAGCAUCGGCCUGGACAUCGGCACCAACAGCGUGGGCUGGGCCG
UGAUCACCGACGAGUACAAGGUGCCCAGCAAGAAGUUCAAGGUGCUGGGCAAC
ACCGACCGGCACAGCAUCAAGAAGAACCUGAUCGGCGCCCUGCUGUUCGACAG
CGGCGAGACCGCCGAGGCCACCCGGCUGAAGCGGACCGCCCGGCGGCGGUACA
CCCGGCGGAAGAACCGGAUCUGCUACCUGCAGGAGAUCUUCAGCAACGAGAUG
GCCAAGGUGGACGACAGCUUCUUCCACCGGCUGGAGGAGAGCUUCCUGGUGGA
GGAGGACAAGAAGCACGAGCGGCACCCCAUCUUCGGCAACAUCGUGGACGAGG
UGGCCUACCACGAGAAGUACCCCACCAUCUACCACCUGCGGAAGAAGCUGGUG
GACAGCACCGACAAGGCCGACCUGCGGCUGAUCUACCUGGCCCUGGCCCACAU
GAUCAAGUUCCGGGGCCACUUCCUGAUCGAGGGCGACCUGAACCCCGACAACA
GCGACGUGGACAAGCUGUUCAUCCAGCUGGUGCAGACCUACAACCAGCUGUUC
GAGGAGAACCCCAUCAACGCCAGCGGCGUGGACGCCAAGGCCAUCCUGAGCGC
CCGGCUGAGCAAGAGCCGGCGGCUGGAGAACCUGAUCGCCCAGCUGCCCGGCG
AGAAGAAGAACGGCCUGUUCGGCAACCUGAUCGCCCUGAGCCUGGGCCUGACC
CCCAACUUCAAGAGCAACUUCGACCUGGCCGAGGACGCCAAGCUGCAGCUGAG
CAAGGACACCUACGACGACGACCUGGACAACCUGCUGGCCCAGAUCGGCGACC
AGUACGCCGACCUGUUCCUGGCCGCCAAGAACCUGAGCGACGCCAUCCUGCUG
AGCGACAUCCUGCGGGUGAACACCGAGAUCACCAAGGCCCCCCUGAGCGCCAG
CAUGAUCAAGCGGUACGACGAGCACCACCAGGACCUGACCCUGCUGAAGGCCC
UGGUGCGGCAGCAGCUGCCCGAGAAGUACAAGGAGAUCUUCUUCGACCAGAGC
AAGAACGGCUACGCCGGCUACAUCGACGGCGGCGCCAGCCAGGAGGAGUUCUA
CAAGUUCAUCAAGCCCAUCCUGGAGAAGAUGGACGGCACCGAGGAGCUGCUGG
UGAAGCUGAACCGGGAGGACCUGCUGCGGAAGCAGCGGACCUUCGACAACGGC
AGCAUCCCCCACCAGAUCCACCUGGGCGAGCUGCACGCCAUCCUGCGGCGGCA
GGAGGACUUCUACCCCUUCCUGAAGGACAACCGGGAGAAGAUCGAGAAGAUCC
UGACCUUCCGGAUCCCCUACUACGUGGGCCCCCUGGCCCGGGGCAACAGCCGG
UUCGCCUGGAUGACCCGGAAGAGCGAGGAGACCAUCACCCCCUGGAACUUCGA
GGAGGUGGUGGACAAGGGCGCCAGCGCCCAGAGCUUCAUCGAGCGGAUGACCA
ACUUCGACAAGAACCUGCCCAACGAGAAGGUGCUGCCCAAGCACAGCCUGCUG
UACGAGUACUUCACCGUGUACAACGAGCUGACCAAGGUGAAGUACGUGACCGA
GGGCAUGCGGAAGCCCGCCUUCCUGAGCGGCGAGCAGAAGAAGGCCAUCGUGG
ACCUGCUGUUCAAGACCAACCGGAAGGUGACCGUGAAGCAGCUGAAGGAGGAC
UACUUCAAGAAGAUCGAGUGCUUCGACAGCGUGGAGAUCAGCGGCGUGGAGG
ACCGGUUCAACGCCAGCCUGGGCACCUACCACGACCUGCUGAAGAUCAUCAAG
GACAAGGACUUCCUGGACAACGAGGAGAACGAGGACAUCCUGGAGGACAUCG
UGCUGACCCUGACCCUGUUCGAGGACCGGGAGAUGAUCGAGGAGCGGCUGAAG
ACCUACGCCCACCUGUUCGACGACAAGGUGAUGAAGCAGCUGAAGCGGCGGCG
GUACACCGGCUGGGGCCGGCUGAGCCGGAAGCUGAUCAACGGCAUCCGGGACA
AGCAGAGCGGCAAGACCAUCCUGGACUUCCUGAAGAGCGACGGCUUCGCCAAC
CGGAACUUCAUGCAGCUGAUCCACGACGACAGCCUGACCUUCAAGGAGGACAU
CCAGAAGGCCCAGGUGAGCGGCCAGGGCGACAGCCUGCACGAGCACAUCGCCA
ACCUGGCCGGCAGCCCCGCCAUCAAGAAGGGCAUCCUGCAGACCGUGAAGGUG
GUGGACGAGCUGGUGAAGGUGAUGGGCCGGCACAAGCCCGAGAACAUCGUGA
UCGAGAUGGCCCGGGAGAACCAGACCACCCAGAAGGGCCAGAAGAACAGCCGG
GAGCGGAUGAAGCGGAUCGAGGAGGGCAUCAAGGAGCUGGGCAGCCAGAUCC
UGAAGGAGCACCCCGUGGAGAACACCCAGCUGCAGAACGAGAAGCUGUACCUG
UACUACCUGCAGAACGGCCGGGACAUGUACGUGGACCAGGAGCUGGACAUCAA
CCGGCUGAGCGACUACGACGUGGACCACAUCGUGCCCCAGAGCUUCCUGAAGG
ACGACAGCAUCGACAACAAGGUGCUGACCCGGAGCGACAAGAACCGGGGCAAG
AGCGACAACGUGCCCAGCGAGGAGGUGGUGAAGAAGAUGAAGAACUACUGGC
GGCAGCUGCUGAACGCCAAGCUGAUCACCCAGCGGAAGUUCGACAACCUGACC
AAGGCCGAGCGGGGCGGCCUGAGCGAGCUGGACAAGGCCGGCUUCAUCAAGCG
GCAGCUGGUGGAGACCCGGCAGAUCACCAAGCACGUGGCCCAGAUCCUGGACA
GCCGGAUGAACACCAAGUACGACGAGAACGACAAGCUGAUCCGGGAGGUGAA
GGUGAUCACCCUGAAGAGCAAGCUGGUGAGCGACUUCCGGAAGGACUUCCAGU
UCUACAAGGUGCGGGAGAUCAACAACUACCACCACGCCCACGACGCCUACCUG
AACGCCGUGGUGGGCACCGCCCUGAUCAAGAAGUACCCCAAGCUGGAGAGCGA
GUUCGUGUACGGCGACUACAAGGUGUACGACGUGCGGAAGAUGAUCGCCAAG
AGCGAGCAGGAGAUCGGCAAGGCCACCGCCAAGUACUUCUUCUACAGCAACAU
CAUGAACUUCUUCAAGACCGAGAUCACCCUGGCCAACGGCGAGAUCCGGAAGC
GGCCCCUGAUCGAGACCAACGGCGAGACCGGCGAGAUCGUGUGGGACAAGGGC
CGGGACUUCGCCACCGUGCGGAAGGUGCUGAGCAUGCCCCAGGUGAACAUCGU
GAAGAAGACCGAGGUGCAGACCGGCGGCUUCAGCAAGGAGAGCAUCCUGCCCA
AGCGGAACAGCGACAAGCUGAUCGCCCGGAAGAAGGACUGGGACCCCAAGAAG
UACGGCGGCUUCGACAGCCCCACCGUGGCCUACAGCGUGCUGGUGGUGGCCAA
GGUGGAGAAGGGCAAGAGCAAGAAGCUGAAGAGCGUGAAGGAGCUGCUGGGC
AUCACCAUCAUGGAGCGGAGCAGCUUCGAGAAGAACCCCAUCGACUUCCUGGA
GGCCAAGGGCUACAAGGAGGUGAAGAAGGACCUGAUCAUCAAGCUGCCCAAG
UACAGCCUGUUCGAGCUGGAGAACGGCCGGAAGCGGAUGCUGGCCAGCGCCGG
CGAGCUGCAGAAGGGCAACGAGCUGGCCCUGCCCAGCAAGUACGUGAACUUCC
UGUACCUGGCCAGCCACUACGAGAAGCUGAAGGGCAGCCCCGAGGACAACGAG
CAGAAGCAGCUGUUCGUGGAGCAGCACAAGCACUACCUGGACGAGAUCAUCGA
GCAGAUCAGCGAGUUCAGCAAGCGGGUGAUCCUGGCCGACGCCAACCUGGACA
AGGUGCUGAGCGCCUACAACAAGCACCGGGACAAGCCCAUCCGGGAGCAGGCC
GAGAACAUCAUCCACCUGUUCACCCUGACCAACCUGGGCGCCCCCGCCGCCUU
CAAGUACUUCGACACCACCAUCGACCGGAAGCGGUACACCAGCACCAAGGAGG
UGCUGGACGCCACCCUGAUCCACCAGAGCAUCACCGGCCUGUACGAGACCCGG
AUCGACCUGAGCCAGCUGGGCGGCGACAGCGGCGGCAAGCGGCCCGCCGCCAC
CAAGAAGGCCGGCCAGGCCAAGAAGAAGAAGGGCAGCUACCCCUACGACGUGC
CCGACUACGCCUGA
SpCas9 ORF #2
[SEQ ID NO: 1481]
AUGGACAAGAAGUACAGCAUCGGCCUGGACAUCGGCACCAACUCUGUGGGCUG
GGCCGUGAUCACCGACGAGUACAAGGUGCCCAGCAAGAAAUUCAAGGUGCUGG
GCAACACCGACCGGCACAGCAUCAAGAAGAACCUGAUCGGAGCCCUGCUGUUC
GACAGCGGCGAAACAGCCGAGGCCACCCGGCUGAAGAGAACCGCCAGAAGAAG
AUACACCAGACGGAAGAACCGGAUCUGCUAUCUGCAAGAGAUCUUCAGCAACG
AGAUGGCCAAGGUGGACGACAGCUUCUUCCACAGACUGGAAGAGUCCUUCCUG
GUGGAAGAGGAUAAGAAGCACGAGCGGCACCCCAUCUUCGGCAACAUCGUGGA
CGAGGUGGCCUACCACGAGAAGUACCCCACCAUCUACCACCUGAGAAAGAAAC
UGGUGGACAGCACCGACAAGGCCGACCUGCGGCUGAUCUAUCUGGCCCUGGCC
CACAUGAUCAAGUUCCGGGGCCACUUCCUGAUCGAGGGCGACCUGAACCCCGA
CAACAGCGACGUGGACAAGCUGUUCAUCCAGCUGGUGCAGACCUACAACCAGC
UGUUCGAGGAAAACCCCAUCAACGCCAGCGGCGUGGACGCCAAGGCCAUCCUG
UCUGCCAGACUGAGCAAGAGCAGACGGCUGGAAAAUCUGAUCGCCCAGCUGCC
CGGCGAGAAGAAGAAUGGCCUGUUCGGAAACCUGAUUGCCCUGAGCCUGGGCC
UGACCCCCAACUUCAAGAGCAACUUCGACCUGGCCGAGGAUGCCAAACUGCAG
CUGAGCAAGGACACCUACGACGACGACCUGGACAACCUGCUGGCCCAGAUCGG
CGACCAGUACGCCGACCUGUUUCUGGCCGCCAAGAACCUGUCCGACGCCAUCC
UGCUGAGCGACAUCCUGAGAGUGAACACCGAGAUCACCAAGGCCCCCCUGAGC
GCCUCUAUGAUCAAGAGAUACGACGAGCACCACCAGGACCUGACCCUGCUGAA
AGCUCUCGUGCGGCAGCAGCUGCCUGAGAAGUACAAAGAGAUUUUCUUCGACC
AGAGCAAGAACGGCUACGCCGGCUACAUUGACGGCGGAGCCAGCCAGGAAGAG
UUCUACAAGUUCAUCAAGCCCAUCCUGGAAAAGAUGGACGGCACCGAGGAACU
GCUCGUGAAGCUGAACAGAGAGGACCUGCUGCGGAAGCAGCGGACCUUCGACA
ACGGCAGCAUCCCCCACCAGAUCCACCUGGGAGAGCUGCACGCCAUUCUGCGG
CGGCAGGAAGAUUUUUACCCAUUCCUGAAGGACAACCGGGAAAAGAUCGAGA
AGAUCCUGACCUUCCGCAUCCCCUACUACGUGGGCCCUCUGGCCAGGGGAAAC
AGCAGAUUCGCCUGGAUGACCAGAAAGAGCGAGGAAACCAUCACCCCCUGGAA
CUUCGAGGAAGUGGUGGACAAGGGCGCUUCCGCCCAGAGCUUCAUCGAGCGGA
UGACCAACUUCGAUAAGAACCUGCCCAACGAGAAGGUGCUGCCCAAGCACAGC
CUGCUGUACGAGUACUUCACCGUGUAUAACGAGCUGACCAAAGUGAAAUACG
UGACCGAGGGAAUGAGAAAGCCCGCCUUCCUGAGCGGCGAGCAGAAAAAGGCC
AUCGUGGACCUGCUGUUCAAGACCAACCGGAAAGUGACCGUGAAGCAGCUGAA
AGAGGACUACUUCAAGAAAAUCGAGUGCUUCGACUCCGUGGAAAUCUCCGGCG
UGGAAGAUCGGUUCAACGCCUCCCUGGGCACAUACCACGAUCUGCUGAAAAUU
AUCAAGGACAAGGACUUCCUGGACAAUGAGGAAAACGAGGACAUUCUGGAAG
AUAUCGUGCUGACCCUGACACUGUUUGAGGACAGAGAGAUGAUCGAGGAACG
GCUGAAAACCUAUGCCCACCUGUUCGACGACAAAGUGAUGAAGCAGCUGAAGC
GGCGGAGAUACACCGGCUGGGGCAGGCUGAGCCGGAAGCUGAUCAACGGCAUC
CGGGACAAGCAGUCCGGCAAGACAAUCCUGGAUUUCCUGAAGUCCGACGGCUU
CGCCAACAGAAACUUCAUGCAGCUGAUCCACGACGACAGCCUGACCUUUAAAG
AGGACAUCCAGAAAGCCCAGGUGUCCGGCCAGGGCGAUAGCCUGCACGAGCAC
AUUGCCAAUCUGGCCGGCAGCCCCGCCAUUAAGAAGGGCAUCCUGCAGACAGU
GAAGGUGGUGGACGAGCUCGUGAAAGUGAUGGGCCGGCACAAGCCCGAGAAC
AUCGUGAUCGAAAUGGCCAGAGAGAACCAGACCACCCAGAAGGGACAGAAGA
ACAGCCGCGAGAGAAUGAAGCGGAUCGAAGAGGGCAUCAAAGAGCUGGGCAG
CCAGAUCCUGAAAGAACACCCCGUGGAAAACACCCAGCUGCAGAACGAGAAGC
UGUACCUGUACUACCUGCAGAAUGGGCGGGAUAUGUACGUGGACCAGGAACU
GGACAUCAACCGGCUGUCCGACUACGAUGUGGACCAUAUCGUGCCUCAGAGCU
UUCUGAAGGACGACUCCAUCGACAACAAGGUGCUGACCAGAAGCGACAAGAAC
CGGGGCAAGAGCGACAACGUGCCCUCCGAAGAGGUCGUGAAGAAGAUGAAGA
ACUACUGGCGGCAGCUGCUGAACGCCAAGCUGAUUACCCAGAGAAAGUUCGAC
AAUCUGACCAAGGCCGAGAGAGGCGGCCUGAGCGAACUGGAUAAGGCCGGCUU
CAUCAAGAGACAGCUGGUGGAAACCCGGCAGAUCACAAAGCACGUGGCACAGA
UCCUGGACUCCCGGAUGAACACUAAGUACGACGAGAAUGACAAGCUGAUCCGG
GAAGUGAAAGUGAUCACCCUGAAGUCCAAGCUGGUGUCCGAUUUCCGGAAGG
AUUUCCAGUUUUACAAAGUGCGCGAGAUCAACAACUACCACCACGCCCACGAC
GCCUACCUGAACGCCGUCGUGGGAACCGCCCUGAUCAAAAAGUACCCUAAGCU
GGAAAGCGAGUUCGUGUACGGCGACUACAAGGUGUACGACGUGCGGAAGAUG
AUCGCCAAGAGCGAGCAGGAAAUCGGCAAGGCUACCGCCAAGUACUUCUUCUA
CAGCAACAUCAUGAACUUUUUCAAGACCGAGAUUACCCUGGCCAACGGCGAGA
UCCGGAAGCGGCCUCUGAUCGAGACAAACGGCGAAACCGGGGAGAUCGUGUGG
GAUAAGGGCCGGGAUUUUGCCACCGUGCGGAAAGUGCUGAGCAUGCCCCAAGU
GAAUAUCGUGAAAAAGACCGAGGUGCAGACAGGCGGCUUCAGCAAAGAGUCU
AUCCUGCCCAAGAGGAACAGCGAUAAGCUGAUCGCCAGAAAGAAGGACUGGG
ACCCUAAGAAGUACGGCGGCUUCGACAGCCCCACCGUGGCCUAUUCUGUGCUG
GUGGUGGCCAAAGUGGAAAAGGGCAAGUCCAAGAAACUGAAGAGUGUGAAAG
AGCUGCUGGGGAUCACCAUCAUGGAAAGAAGCAGCUUCGAGAAGAAUCCCAUC
GACUUUCUGGAAGCCAAGGGCUACAAAGAAGUGAAAAAGGACCUGAUCAUCA
AGCUGCCUAAGUACUCCCUGUUCGAGCUGGAAAACGGCCGGAAGAGAAUGCUG
GCCUCUGCCGGCGAACUGCAGAAGGGAAACGAACUGGCCCUGCCCUCCAAAUA
UGUGAACUUCCUGUACCUGGCCAGCCACUAUGAGAAGCUGAAGGGCUCCCCCG
AGGAUAAUGAGCAGAAACAGCUGUUUGUGGAACAGCACAAGCACUACCUGGA
CGAGAUCAUCGAGCAGAUCAGCGAGUUCUCCAAGAGAGUGAUCCUGGCCGACG
CUAAUCUGGACAAAGUGCUGUCCGCCUACAACAAGCACCGGGAUAAGCCCAUC
AGAGAGCAGGCCGAGAAUAUCAUCCACCUGUUUACCCUGACCAAUCUGGGAGC
CCCUGCCGCCUUCAAGUACUUUGACACCACCAUCGACCGGAAGAGGUACACCA
GCACCAAAGAGGUGCUGGACGCCACCCUGAUCCACCAGAGCAUCACCGGCCUG
UACGAGACACGGAUCGACCUGUCUCAGCUGGGAGGUGACGGCGGAUCCCCUGC
UGCUAAACGUGUUAAGCUUGAUGGGGGUAGCCCGGCAGCCAAGAGAGUCAAA
CUCGACUAGAUGGUUUAUAUUGCGGCCGCUUAAUUAA
SpCas9 mRNA #1
[SEQ ID NO: 1350]
GGGTCCCGCAGTCGGCGTCCAGCGGCTCTGCTTGTTCGTGTGTGTGTCGTTGCAG
GCCTTATTCGGATCCGCCACCATGCCTAAGAAAAAGCGGAAGGTCGACGGGGAT
AAGAAGTACTCAATCGGGCTGGATATCGGAACTAATTCCGTGGGTTGGGCAGTG
ATCACGGATGAATACAAAGTGCCGTCCAAGAAGTTCAAGGTCCTGGGGAACACC
GATAGACACAGCATCAAGAAAAATCTCATCGGAGCCCTGCTGTTTGACTCCGGC
GAAACCGCAGAAGCGACCCGGCTCAAACGTACCGCGAGGCGACGCTACACCCGG
CGGAAGAATCGCATCTGCTATCTGCAAGAGATCTTTTCGAACGAAATGGCAAAG
GTCGACGACAGCTTCTTCCACCGCCTGGAAGAATCTTTCCTGGTGGAGGAGGACA
AGAAGCATGAACGGCATCCTATCTTTGGAAACATCGTCGACGAAGTGGCGTACC
ACGAAAAGTACCCGACCATCTACCATCTGCGGAAGAAGTTGGTTGACTCAACTG
ACAAGGCCGACCTCAGATTGATCTACTTGGCCCTCGCCCATATGATCAAATTCCG
CGGACACTTCCTGATCGAAGGCGATCTGAACCCTGATAACTCCGACGTGGATAA
GCTTTTCATTCAACTGGTGCAGACCTACAACCAACTGTTCGAAGAAAACCCAATC
AATGCTAGCGGCGTCGATGCCAAGGCCATCCTGTCCGCCCGGCTGTCGAAGTCGC
GGCGCCTCGAAAACCTGATCGCACAGCTGCCGGGAGAGAAAAAGAACGGACTTT
TCGGCAACTTGATCGCTCTCTCACTGGGACTCACTCCCAATTTCAAGTCCAATTTT
GACCTGGCCGAGGACGCGAAGCTGCAACTCTCAAAGGACACCTACGACGACGAC
TTGGACAATTTGCTGGCACAAATTGGCGATCAGTACGCGGATCTGTTCCTTGCCG
CTAAGAACCTTTCGGACGCAATCTTGCTGTCCGATATCCTGCGCGTGAACACCGA
AATAACCAAAGCGCCGCTTAGCGCCTCGATGATTAAGCGGTACGACGAGCATCA
CCAGGATCTCACGCTGCTCAAAGCGCTCGTGAGACAGCAACTGCCTGAAAAGTA
CAAGGAGATCTTCTTCGACCAGTCCAAGAATGGGTACGCAGGGTACATCGATGG
AGGCGCTAGCCAGGAAGAGTTCTATAAGTTCATCAAGCCAATCCTGGAAAAGAT
GGACGGAACCGAAGAACTGCTGGTCAAGCTGAACAGGGAGGATCTGCTCCGGAA
ACAGAGAACCTTTGACAACGGATCCATTCCCCACCAGATCCATCTGGGTGAGCTG
CACGCCATCTTGCGGCGCCAGGAGGACTTTTACCCATTCCTCAAGGACAACCGGG
AAAAGATCGAGAAAATTCTGACGTTCCGCATCCCGTATTACGTGGGCCCACTGGC
GCGCGGCAATTCGCGCTTCGCGTGGATGACTAGAAAATCAGAGGAAACCATCAC
TCCTTGGAATTTCGAGGAAGTTGTGGATAAGGGAGCTTCGGCACAAAGCTTCATC
GAACGAATGACCAACTTCGACAAGAATCTCCCAAACGAGAAGGTGCTTCCTAAG
CACAGCCTCCTTTACGAATACTTCACTGTCTACAACGAACTGACTAAAGTGAAAT
ACGTTACTGAAGGAATGAGGAAGCCGGCCTTTCTGTCCGGAGAACAGAAGAAAG
CAATTGTCGATCTGCTGTTCAAGACCAACCGCAAGGTGACCGTCAAGCAGCTTAA
AGAGGACTACTTCAAGAAGATCGAGTGTTTCGACTCAGTGGAAATCAGCGGGGT
GGAGGACAGATTCAACGCTTCGCTGGGAACCTATCATGATCTCCTGAAGATCATC
AAGGACAAGGACTTCCTTGACAACGAGGAGAACGAGGACATCCTGGAAGATATC
GTCCTGACCTTGACCCTTTTCGAGGATCGCGAGATGATCGAGGAGAGGCTTAAG
ACCTACGCTCATCTCTTCGACGATAAGGTCATGAAACAACTCAAGCGCCGCCGGT
ACACTGGTTGGGGCCGCCTCTCCCGCAAGCTGATCAACGGTATTCGCGATAAACA
GAGCGGTAAAACTATCCTGGATTTCCTCAAATCGGATGGCTTCGCTAATCGTAAC
TTCATGCAATTGATCCACGACGACAGCCTGACCTTTAAGGAGGACATCCAAAAA
GCACAAGTGTCCGGACAGGGAGACTCACTCCATGAACACATCGCGAATCTGGCC
GGTTCGCCGGCGATTAAGAAGGGAATTCTGCAAACTGTGAAGGTGGTCGACGAG
CTGGTGAAGGTCATGGGACGGCACAAACCGGAGAATATCGTGATTGAAATGGCC
CGAGAAAACCAGACTACCCAGAAGGGCCAGAAAAACTCCCGCGAAAGGATGAA
GCGGATCGAAGAAGGAATCAAGGAGCTGGGCAGCCAGATCCTGAAAGAGCACC
CGGTGGAAAACACGCAGCTGCAGAACGAGAAGCTCTACCTGTACTATTTGCAAA
ATGGACGGGACATGTACGTGGACCAAGAGCTGGACATCAATCGGTTGTCTGATT
ACGACGTGGACCACATCGTTCCACAGTCCTTTCTGAAGGATGACTCGATCGATAA
CAAGGTGTTGACTCGCAGCGACAAGAACAGAGGGAAGTCAGATAATGTGCCATC
GGAGGAGGTCGTGAAGAAGATGAAGAATTACTGGCGGCAGCTCCTGAATGCGAA
GCTGATTACCCAGAGAAAGTTTGACAATCTCACTAAAGCCGAGCGCGGCGGACT
CTCAGAGCTGGATAAGGCTGGATTCATCAAACGGCAGCTGGTCGAGACTCGGCA
GATTACCAAGCACGTGGCGCAGATCTTGGACTCCCGCATGAACACTAAATACGA
CGAGAACGATAAGCTCATCCGGGAAGTGAAGGTGATTACCCTGAAAAGCAAACT
TGTGTCGGACTTTCGGAAGGACTTTCAGTTTTACAAAGTGAGAGAAATCAACAAC
TACCATCACGCGCATGACGCATACCTCAACGCTGTGGTCGGTACCGCCCTGATCA
AAAAGTACCCTAAACTTGAATCGGAGTTTGTGTACGGAGACTACAAGGTCTACG
ACGTGAGGAAGATGATAGCCAAGTCCGAACAGGAAATCGGGAAAGCAACTGCG
AAATACTTCTTTTACTCAAACATCATGAACTTTTTCAAGACTGAAATTACGCTGG
CCAATGGAGAAATCAGGAAGAGGCCACTGATCGAAACTAACGGAGAAACGGGC
GAAATCGTGTGGGACAAGGGCAGGGACTTCGCAACTGTTCGCAAAGTGCTCTCT
ATGCCGCAAGTCAATATTGTGAAGAAAACCGAAGTGCAAACCGGCGGATTTTCA
AAGGAATCGATCCTCCCAAAGAGAAATAGCGACAAGCTCATTGCACGCAAGAAA
GACTGGGACCCGAAGAAGTACGGAGGATTCGATTCGCCGACTGTCGCATACTCC
GTCCTCGTGGTGGCCAAGGTGGAGAAGGGAAAGAGCAAAAAGCTCAAATCCGTC
AAAGAGCTGCTGGGGATTACCATCATGGAACGATCCTCGTTCGAGAAGAACCCG
ATTGATTTCCTCGAGGCGAAGGGTTACAAGGAGGTGAAGAAGGATCTGATCATC
AAACTCCCCAAGTACTCACTGTTCGAACTGGAAAATGGTCGGAAGCGCATGCTG
GCTTCGGCCGGAGAACTCCAAAAAGGAAATGAGCTGGCCTTGCCTAGCAAGTAC
GTCAACTTCCTCTATCTTGCTTCGCACTACGAAAAACTCAAAGGGTCACCGGAAG
ATAACGAACAGAAGCAGCTTTTCGTGGAGCAGCACAAGCATTATCTGGATGAAA
TCATCGAACAAATCTCCGAGTTTTCAAAGCGCGTGATCCTCGCCGACGCCAACCT
CGACAAAGTCCTGTCGGCCTACAATAAGCATAGAGATAAGCCGATCAGAGAACA
GGCCGAGAACATTATCCACTTGTTCACCCTGACTAACCTGGGAGCCCCAGCCGCC
TTCAAGTACTTCGATACTACTATCGATCGCAAAAGATACACGTCCACCAAGGAA
GTTCTGGACGCGACCCTGATCCACCAAAGCATCACTGGACTCTACGAAACTAGG
ATCGATCTGTCGCAGCTGGGTGGCGATTGATAGTCTAGCCATCACATTTAAAAGC
ATCTCAGCCTACCATGAGAATAAGAGAAAGAAAATGAAGATCAATAGCTTATTC
ATCTCTTTTTCTTTTTCGTTGGTGTAAAGCCAACACCCTGTCTAAAAAACATAAAT
TTCTTTAATCATTTTGCCTCTTTTCTCTGTGCTTCAATTAATAAAAAATGGAAAGA
ACCTCGAG
SpCas9 mRNA #2
[SEQ ID NO: 1351]
GGGTCCCGCAGTCGGCGTCCAGCGGCTCTGCTTGTTCGTGTGTGTGTCGTTGCAG
GCCTTATTCGGATCCGCCACCATGGACAAGAAGTACAGCATCGGACTGGACATC
GGAACAAACAGCGTCGGATGGGCAGTCATCACAGACGAATACAAGGTCCCGAGC
AAGAAGTTCAAGGTCCTGGGAAACACAGACAGACACAGCATCAAGAAGAACCT
GATCGGAGCACTGCTGTTCGACAGCGGAGAAACAGCAGAAGCAACAAGACTGA
AGAGAACAGCAAGAAGAAGATACACAAGAAGAAAGAACAGAATCTGCTACCTG
CAGGAAATCTTCAGCAACGAAATGGCAAAGGTCGACGACAGCTTCTTCCACCGG
CTGGAAGAAAGCTTCCTGGTCGAAGAAGACAAGAAGCACGAAAGACACCCGAT
CTTCGGAAACATCGTCGACGAAGTCGCATACCACGAAAAGTACCCGACAATCTA
CCACCTGAGAAAGAAGCTGGTCGACAGCACAGACAAGGCAGACCTGAGACTGAT
CTACCTGGCACTGGCACACATGATCAAGTTCAGAGGACACTTCCTGATCGAAGG
AGACCTGAACCCGGACAACAGCGACGTCGACAAGCTGTTCATCCAGCTGGTCCA
GACATACAACCAGCTGTTCGAAGAAAACCCGATCAACGCAAGCGGAGTCGACGC
AAAGGCAATCCTGAGCGCAAGACTGAGCAAGAGCAGAAGACTGGAAAACCTGA
TCGCACAGCTGCCGGGAGAAAAGAAGAACGGACTGTTCGGAAACCTGATCGCAC
TGAGCCTGGGACTGACACCGAACTTCAAGAGCAACTTCGACCTGGCAGAAGACG
CAAAGCTGCAGCTGAGCAAGGACACATACGACGACGACCTGGACAACCTGCTGG
CACAGATCGGAGACCAGTACGCAGACCTGTTCCTGGCAGCAAAGAACCTGAGCG
ACGCAATCCTGCTGAGCGACATCCTGAGAGTCAACACAGAAATCACAAAGGCAC
CGCTGAGCGCAAGCATGATCAAGAGATACGACGAACACCACCAGGACCTGACAC
TGCTGAAGGCACTGGTCAGACAGCAGCTGCCGGAAAAGTACAAGGAAATCTTCT
TCGACCAGAGCAAGAACGGATACGCAGGATACATCGACGGAGGAGCAAGCCAG
GAAGAATTCTACAAGTTCATCAAGCCGATCCTGGAAAAGATGGACGGAACAGAA
GAACTGCTGGTCAAGCTGAACAGAGAAGACCTGCTGAGAAAGCAGAGAACATTC
GACAACGGAAGCATCCCGCACCAGATCCACCTGGGAGAACTGCACGCAATCCTG
AGAAGACAGGAAGACTTCTACCCGTTCCTGAAGGACAACAGAGAAAAGATCGA
AAAGATCCTGACATTCAGAATCCCGTACTACGTCGGACCGCTGGCAAGAGGAAA
CAGCAGATTCGCATGGATGACAAGAAAGAGCGAAGAAACAATCACACCGTGGA
ACTTCGAAGAAGTCGTCGACAAGGGAGCAAGCGCACAGAGCTTCATCGAAAGAA
TGACAAACTTCGACAAGAACCTGCCGAACGAAAAGGTCCTGCCGAAGCACAGCC
TGCTGTACGAATACTTCACAGTCTACAACGAACTGACAAAGGTCAAGTACGTCA
CAGAAGGAATGAGAAAGCCGGCATTCCTGAGCGGAGAACAGAAGAAGGCAATC
GTCGACCTGCTGTTCAAGACAAACAGAAAGGTCACAGTCAAGCAGCTGAAGGAA
GACTACTTCAAGAAGATCGAATGCTTCGACAGCGTCGAAATCAGCGGAGTCGAA
GACAGATTCAACGCAAGCCTGGGAACATACCACGACCTGCTGAAGATCATCAAG
GACAAGGACTTCCTGGACAACGAAGAAAACGAAGACATCCTGGAAGACATCGTC
CTGACACTGACACTGTTCGAAGACAGAGAAATGATCGAAGAAAGACTGAAGACA
TACGCACACCTGTTCGACGACAAGGTCATGAAGCAGCTGAAGAGAAGAAGATAC
ACAGGATGGGGAAGACTGAGCAGAAAGCTGATCAACGGAATCAGAGACAAGCA
GAGCGGAAAGACAATCCTGGACTTCCTGAAGAGCGACGGATTCGCAAACAGAAA
CTTCATGCAGCTGATCCACGACGACAGCCTGACATTCAAGGAAGACATCCAGAA
GGCACAGGTCAGCGGACAGGGAGACAGCCTGCACGAACACATCGCAAACCTGG
CAGGAAGCCCGGCAATCAAGAAGGGAATCCTGCAGACAGTCAAGGTCGTCGACG
AACTGGTCAAGGTCATGGGAAGACACAAGCCGGAAAACATCGTCATCGAAATGG
CAAGAGAAAACCAGACAACACAGAAGGGACAGAAGAACAGCAGAGAAAGAAT
GAAGAGAATCGAAGAAGGAATCAAGGAACTGGGAAGCCAGATCCTGAAGGAAC
ACCCGGTCGAAAACACACAGCTGCAGAACGAAAAGCTGTACCTGTACTACCTGC
AAAACGGAAGAGACATGTACGTCGACCAGGAACTGGACATCAACAGACTGAGC
GACTACGACGTCGACCACATCGTCCCGCAGAGCTTCCTGAAGGACGACAGCATC
GACAACAAGGTCCTGACAAGAAGCGACAAGAACAGAGGAAAGAGCGACAACGT
CCCGAGCGAAGAAGTCGTCAAGAAGATGAAGAACTACTGGAGACAGCTGCTGA
ACGCAAAGCTGATCACACAGAGAAAGTTCGACAACCTGACAAAGGCAGAGAGA
GGAGGACTGAGCGAACTGGACAAGGCAGGATTCATCAAGAGACAGCTGGTCGA
AACAAGACAGATCACAAAGCACGTCGCACAGATCCTGGACAGCAGAATGAACA
CAAAGTACGACGAAAACGACAAGCTGATCAGAGAAGTCAAGGTCATCACACTGA
AGAGCAAGCTGGTCAGCGACTTCAGAAAGGACTTCCAGTTCTACAAGGTCAGAG
AAATCAACAACTACCACCACGCACACGACGCATACCTGAACGCAGTCGTCGGAA
CAGCACTGATCAAGAAGTACCCGAAGCTGGAAAGCGAATTCGTCTACGGAGACT
ACAAGGTCTACGACGTCAGAAAGATGATCGCAAAGAGCGAACAGGAAATCGGA
AAGGCAACAGCAAAGTACTTCTTCTACAGCAACATCATGAACTTCTTCAAGACA
GAAATCACACTGGCAAACGGAGAAATCAGAAAGAGACCGCTGATCGAAACAAA
CGGAGAAACAGGAGAAATCGTCTGGGACAAGGGAAGAGACTTCGCAACAGTCA
GAAAGGTCCTGAGCATGCCGCAGGTCAACATCGTCAAGAAGACAGAAGTCCAGA
CAGGAGGATTCAGCAAGGAAAGCATCCTGCCGAAGAGAAACAGCGACAAGCTG
ATCGCAAGAAAGAAGGACTGGGACCCGAAGAAGTACGGAGGATTCGACAGCCC
GACAGTCGCATACAGCGTCCTGGTCGTCGCAAAGGTCGAAAAGGGAAAGAGCAA
GAAGCTGAAGAGCGTCAAGGAACTGCTGGGAATCACAATCATGGAAAGAAGCA
GCTTCGAAAAGAACCCGATCGACTTCCTGGAAGCAAAGGGATACAAGGAAGTCA
AGAAGGACCTGATCATCAAGCTGCCGAAGTACAGCCTGTTCGAACTGGAAAACG
GAAGAAAGAGAATGCTGGCAAGCGCAGGAGAACTGCAGAAGGGAAACGAACTG
GCACTGCCGAGCAAGTACGTCAACTTCCTGTACCTGGCAAGCCACTACGAAAAG
CTGAAGGGAAGCCCGGAAGACAACGAACAGAAGCAGCTGTTCGTCGAACAGCA
CAAGCACTACCTGGACGAAATCATCGAACAGATCAGCGAATTCAGCAAGAGAGT
CATCCTGGCAGACGCAAACCTGGACAAGGTCCTGAGCGCATACAACAAGCACAG
AGACAAGCCGATCAGAGAACAGGCAGAAAACATCATCCACCTGTTCACACTGAC
AAACCTGGGAGCACCGGCAGCATTCAAGTACTTCGACACAACAATCGACAGAAA
GAGATACACAAGCACAAAGGAAGTCCTGGACGCAACACTGATCCACCAGAGCAT
CACAGGACTGTACGAAACAAGAATCGACCTGAGCCAGCTGGGAGGAGACGGAG
GAGGAAGCCCGAAGAAGAAGAGAAAGGTCTAGCTAGCCATCACATTTAAAAGC
ATCTCAGCCTACCATGAGAATAAGAGAAAGAAAATGAAGATCAATAGCTTATTC
ATCTCTTTTTCTTTTTCGTTGGTGTAAAGCCAACACCCTGTCTAAAAAACATAAAT
TTCTTTAATCATTTTGCCTCTTTTCTCTGTGCTTCAATTAATAAAAAATGGAAAGA
ACCTCGAG
SpCas9 mRNA #3
[SEQ ID NO: 1352]
GGGTCCCGCAGTCGGCGTCCAGCGGCTCTGCTTGTTCGTGTGTGTGTCGTTGCAG
GCCTTATTCGGATCCGCCACCATGGACAAGAAGTACAGCATCGGCCTGGACATC
GGCACCAACAGCGTGGGCTGGGCCGTGATCACCGACGAGTACAAGGTGCCCAGC
AAGAAGTTCAAGGTGCTGGGCAACACCGACAGACACAGCATCAAGAAGAACCT
GATCGGCGCCCTGCTGTTCGACAGCGGCGAGACCGCCGAGGCCACCAGACTGAA
GAGAACCGCCAGAAGAAGATACACCAGAAGAAAGAACAGAATCTGCTACCTGC
AGGAGATCTTCAGCAACGAGATGGCCAAGGTGGACGACAGCTTCTTCCACAGAC
TGGAGGAGAGCTTCCTGGTGGAGGAGGACAAGAAGCACGAGAGACACCCCATCT
TCGGCAACATCGTGGACGAGGTGGCCTACCACGAGAAGTACCCCACCATCTACC
ACCTGAGAAAGAAGCTGGTGGACAGCACCGACAAGGCCGACCTGAGACTGATCT
ACCTGGCCCTGGCCCACATGATCAAGTTCAGAGGCCACTTCCTGATCGAGGGCG
ACCTGAACCCCGACAACAGCGACGTGGACAAGCTGTTCATCCAGCTGGTGCAGA
CCTACAACCAGCTGTTCGAGGAGAACCCCATCAACGCCAGCGGCGTGGACGCCA
AGGCCATCCTGAGCGCCAGACTGAGCAAGAGCAGAAGACTGGAGAACCTGATCG
CCCAGCTGCCCGGCGAGAAGAAGAACGGCCTGTTCGGCAACCTGATCGCCCTGA
GCCTGGGCCTGACCCCCAACTTCAAGAGCAACTTCGACCTGGCCGAGGACGCCA
AGCTGCAGCTGAGCAAGGACACCTACGACGACGACCTGGACAACCTGCTGGCCC
AGATCGGCGACCAGTACGCCGACCTGTTCCTGGCCGCCAAGAACCTGAGCGACG
CCATCCTGCTGAGCGACATCCTGAGAGTGAACACCGAGATCACCAAGGCCCCCC
TGAGCGCCAGCATGATCAAGAGATACGACGAGCACCACCAGGACCTGACCCTGC
TGAAGGCCCTGGTGAGACAGCAGCTGCCCGAGAAGTACAAGGAGATCTTCTTCG
ACCAGAGCAAGAACGGCTACGCCGGCTACATCGACGGCGGCGCCAGCCAGGAG
GAGTTCTACAAGTTCATCAAGCCCATCCTGGAGAAGATGGACGGCACCGAGGAG
CTGCTGGTGAAGCTGAACAGAGAGGACCTGCTGAGAAAGCAGAGAACCTTCGAC
AACGGCAGCATCCCCCACCAGATCCACCTGGGCGAGCTGCACGCCATCCTGAGA
AGACAGGAGGACTTCTACCCCTTCCTGAAGGACAACAGAGAGAAGATCGAGAAG
ATCCTGACCTTCAGAATCCCCTACTACGTGGGCCCCCTGGCCAGAGGCAACAGCA
GATTCGCCTGGATGACCAGAAAGAGCGAGGAGACCATCACCCCCTGGAACTTCG
AGGAGGTGGTGGACAAGGGCGCCAGCGCCCAGAGCTTCATCGAGAGAATGACC
AACTTCGACAAGAACCTGCCCAACGAGAAGGTGCTGCCCAAGCACAGCCTGCTG
TACGAGTACTTCACCGTGTACAACGAGCTGACCAAGGTGAAGTACGTGACCGAG
GGCATGAGAAAGCCCGCCTTCCTGAGCGGCGAGCAGAAGAAGGCCATCGTGGAC
CTGCTGTTCAAGACCAACAGAAAGGTGACCGTGAAGCAGCTGAAGGAGGACTAC
TTCAAGAAGATCGAGTGCTTCGACAGCGTGGAGATCAGCGGCGTGGAGGACAGA
TTCAACGCCAGCCTGGGCACCTACCACGACCTGCTGAAGATCATCAAGGACAAG
GACTTCCTGGACAACGAGGAGAACGAGGACATCCTGGAGGACATCGTGCTGACC
CTGACCCTGTTCGAGGACAGAGAGATGATCGAGGAGAGACTGAAGACCTACGCC
CACCTGTTCGACGACAAGGTGATGAAGCAGCTGAAGAGAAGAAGATACACCGGC
TGGGGCAGACTGAGCAGAAAGCTGATCAACGGCATCAGAGACAAGCAGAGCGG
CAAGACCATCCTGGACTTCCTGAAGAGCGACGGCTTCGCCAACAGAAACTTCAT
GCAGCTGATCCACGACGACAGCCTGACCTTCAAGGAGGACATCCAGAAGGCCCA
GGTGAGCGGCCAGGGCGACAGCCTGCACGAGCACATCGCCAACCTGGCCGGCAG
CCCCGCCATCAAGAAGGGCATCCTGCAGACCGTGAAGGTGGTGGACGAGCTGGT
GAAGGTGATGGGCAGACACAAGCCCGAGAACATCGTGATCGAGATGGCCAGAG
AGAACCAGACCACCCAGAAGGGCCAGAAGAACAGCAGAGAGAGAATGAAGAGA
ATCGAGGAGGGCATCAAGGAGCTGGGCAGCCAGATCCTGAAGGAGCACCCCGTG
GAGAACACCCAGCTGCAGAACGAGAAGCTGTACCTGTACTACCTGCAGAACGGC
AGAGACATGTACGTGGACCAGGAGCTGGACATCAACAGACTGAGCGACTACGAC
GTGGACCACATCGTGCCCCAGAGCTTCCTGAAGGACGACAGCATCGACAACAAG
GTGCTGACCAGAAGCGACAAGAACAGAGGCAAGAGCGACAACGTGCCCAGCGA
GGAGGTGGTGAAGAAGATGAAGAACTACTGGAGACAGCTGCTGAACGCCAAGC
TGATCACCCAGAGAAAGTTCGACAACCTGACCAAGGCCGAGAGAGGCGGCCTGA
GCGAGCTGGACAAGGCCGGCTTCATCAAGAGACAGCTGGTGGAGACCAGACAG
ATCACCAAGCACGTGGCCCAGATCCTGGACAGCAGAATGAACACCAAGTACGAC
GAGAACGACAAGCTGATCAGAGAGGTGAAGGTGATCACCCTGAAGAGCAAGCT
GGTGAGCGACTTCAGAAAGGACTTCCAGTTCTACAAGGTGAGAGAGATCAACAA
CTACCACCACGCCCACGACGCCTACCTGAACGCCGTGGTGGGCACCGCCCTGATC
AAGAAGTACCCCAAGCTGGAGAGCGAGTTCGTGTACGGCGACTACAAGGTGTAC
GACGTGAGAAAGATGATCGCCAAGAGCGAGCAGGAGATCGGCAAGGCCACCGC
CAAGTACTTCTTCTACAGCAACATCATGAACTTCTTCAAGACCGAGATCACCCTG
GCCAACGGCGAGATCAGAAAGAGACCCCTGATCGAGACCAACGGCGAGACCGG
CGAGATCGTGTGGGACAAGGGCAGAGACTTCGCCACCGTGAGAAAGGTGCTGAG
CATGCCCCAGGTGAACATCGTGAAGAAGACCGAGGTGCAGACCGGCGGCTTCAG
CAAGGAGAGCATCCTGCCCAAGAGAAACAGCGACAAGCTGATCGCCAGAAAGA
AGGACTGGGACCCCAAGAAGTACGGCGGCTTCGACAGCCCCACCGTGGCCTACA
GCGTGCTGGTGGTGGCCAAGGTGGAGAAGGGCAAGAGCAAGAAGCTGAAGAGC
GTGAAGGAGCTGCTGGGCATCACCATCATGGAGAGAAGCAGCTTCGAGAAGAAC
CCCATCGACTTCCTGGAGGCCAAGGGCTACAAGGAGGTGAAGAAGGACCTGATC
ATCAAGCTGCCCAAGTACAGCCTGTTCGAGCTGGAGAACGGCAGAAAGAGAATG
CTGGCCAGCGCCGGCGAGCTGCAGAAGGGCAACGAGCTGGCCCTGCCCAGCAAG
TACGTGAACTTCCTGTACCTGGCCAGCCACTACGAGAAGCTGAAGGGCAGCCCC
GAGGACAACGAGCAGAAGCAGCTGTTCGTGGAGCAGCACAAGCACTACCTGGAC
GAGATCATCGAGCAGATCAGCGAGTTCAGCAAGAGAGTGATCCTGGCCGACGCC
AACCTGGACAAGGTGCTGAGCGCCTACAACAAGCACAGAGACAAGCCCATCAGA
GAGCAGGCCGAGAACATCATCCACCTGTTCACCCTGACCAACCTGGGCGCCCCC
GCCGCCTTCAAGTACTTCGACACCACCATCGACAGAAAGAGATACACCAGCACC
AAGGAGGTGCTGGACGCCACCCTGATCCACCAGAGCATCACCGGCCTGTACGAG
ACCAGAATCGACCTGAGCCAGCTGGGCGGCGACGGCGGCGGCAGCCCCAAGAA
GAAGAGAAAGGTGTGACTAGCCATCACATTTAAAAGCATCTCAGCCTACCATGA
GAATAAGAGAAAGAAAATGAAGATCAATAGCTTATTCATCTCTTTTTCTTTTTCG
TTGGTGTAAAGCCAACACCCTGTCTAAAAAACATAAATTTCTTTAATCATTTTGC
CTCTTTTCTCTGTGCTTCAATTAATAAAAAATGGAAAGAACCTCGAG
SpCas9 mRNA #4
[SEQ ID NO: 1353]
GGGTCCCGCAGTCGGCGTCCAGCGGCTCTGCTTGTTCGTGTGTGTGTCGTTGCAG
GCCTTATTCGGATCCGCCACCATGGACAAAAAATACAGCATAGGGCTAGACATA
GGGACGAACAGCGTAGGGTGGGCGGTAATAACGGACGAATACAAAGTACCGAG
CAAAAAATTCAAAGTACTAGGGAACACGGACCGACACAGCATAAAAAAAAACC
TAATAGGGGCGCTACTATTCGACAGCGGGGAAACGGCGGAAGCGACGCGACTAA
AACGAACGGCGCGACGACGATACACGCGACGAAAAAACCGAATATGCTACCTAC
AAGAAATATTCAGCAACGAAATGGCGAAAGTAGACGACAGCTTCTTCCACCGAC
TAGAAGAAAGCTTCCTAGTAGAAGAAGACAAAAAACACGAACGACACCCGATA
TTCGGGAACATAGTAGACGAAGTAGCGTACCACGAAAAATACCCGACGATATAC
CACCTACGAAAAAAACTAGTAGACAGCACGGACAAAGCGGACCTACGACTAATA
TACCTAGCGCTAGCGCACATGATAAAATTCCGAGGGCACTTCCTAATAGAAGGG
GACCTAAACCCGGACAACAGCGACGTAGACAAACTATTCATACAACTAGTACAA
ACGTACAACCAACTATTCGAAGAAAACCCGATAAACGCGAGCGGGGTAGACGCG
AAAGCGATACTAAGCGCGCGACTAAGCAAAAGCCGACGACTAGAAAACCTAAT
AGCGCAACTACCGGGGGAAAAAAAAAACGGGCTATTCGGGAACCTAATAGCGCT
AAGCCTAGGGCTAACGCCGAACTTCAAAAGCAACTTCGACCTAGCGGAAGACGC
GAAACTACAACTAAGCAAAGACACGTACGACGACGACCTAGACAACCTACTAGC
GCAAATAGGGGACCAATACGCGGACCTATTCCTAGCGGCGAAAAACCTAAGCGA
CGCGATACTACTAAGCGACATACTACGAGTAAACACGGAAATAACGAAAGCGCC
GCTAAGCGCGAGCATGATAAAACGATACGACGAACACCACCAAGACCTAACGCT
ACTAAAAGCGCTAGTACGACAACAACTACCGGAAAAATACAAAGAAATATTCTT
CGACCAAAGCAAAAACGGGTACGCGGGGTACATAGACGGGGGGGCGAGCCAAG
AAGAATTCTACAAATTCATAAAACCGATACTAGAAAAAATGGACGGGACGGAAG
AACTACTAGTAAAACTAAACCGAGAAGACCTACTACGAAAACAACGAACGTTCG
ACAACGGGAGCATACCGCACCAAATACACCTAGGGGAACTACACGCGATACTAC
GACGACAAGAAGACTTCTACCCGTTCCTAAAAGACAACCGAGAAAAAATAGAAA
AAATACTAACGTTCCGAATACCGTACTACGTAGGGCCGCTAGCGCGAGGGAACA
GCCGATTCGCGTGGATGACGCGAAAAAGCGAAGAAACGATAACGCCGTGGAACT
TCGAAGAAGTAGTAGACAAAGGGGCGAGCGCGCAAAGCTTCATAGAACGAATG
ACGAACTTCGACAAAAACCTACCGAACGAAAAAGTACTACCGAAACACAGCCTA
CTATACGAATACTTCACGGTATACAACGAACTAACGAAAGTAAAATACGTAACG
GAAGGGATGCGAAAACCGGCGTTCCTAAGCGGGGAACAAAAAAAAGCGATAGT
AGACCTACTATTCAAAACGAACCGAAAAGTAACGGTAAAACAACTAAAAGAAG
ACTACTTCAAAAAAATAGAATGCTTCGACAGCGTAGAAATAAGCGGGGTAGAAG
ACCGATTCAACGCGAGCCTAGGGACGTACCACGACCTACTAAAAATAATAAAAG
ACAAAGACTTCCTAGACAACGAAGAAAACGAAGACATACTAGAAGACATAGTA
CTAACGCTAACGCTATTCGAAGACCGAGAAATGATAGAAGAACGACTAAAAACG
TACGCGCACCTATTCGACGACAAAGTAATGAAACAACTAAAACGACGACGATAC
ACGGGGTGGGGGCGACTAAGCCGAAAACTAATAAACGGGATACGAGACAAACA
AAGCGGGAAAACGATACTAGACTTCCTAAAAAGCGACGGGTTCGCGAACCGAAA
CTTCATGCAACTAATACACGACGACAGCCTAACGTTCAAAGAAGACATACAAAA
AGCGCAAGTAAGCGGGCAAGGGGACAGCCTACACGAACACATAGCGAACCTAG
CGGGGAGCCCGGCGATAAAAAAAGGGATACTACAAACGGTAAAAGTAGTAGAC
GAACTAGTAAAAGTAATGGGGCGACACAAACCGGAAAACATAGTAATAGAAAT
GGCGCGAGAAAACCAAACGACGCAAAAAGGGCAAAAAAACAGCCGAGAACGA
ATGAAACGAATAGAAGAAGGGATAAAAGAACTAGGGAGCCAAATACTAAAAGA
ACACCCGGTAGAAAACACGCAACTACAAAACGAAAAACTATACCTATACTACCT
ACAAAACGGGCGAGACATGTACGTAGACCAAGAACTAGACATAAACCGACTAA
GCGACTACGACGTAGACCACATAGTACCGCAAAGCTTCCTAAAAGACGACAGCA
TAGACAACAAAGTACTAACGCGAAGCGACAAAAACCGAGGGAAAAGCGACAAC
GTACCGAGCGAAGAAGTAGTAAAAAAAATGAAAAACTACTGGCGACAACTACT
AAACGCGAAACTAATAACGCAACGAAAATTCGACAACCTAACGAAAGCGGAAC
GAGGGGGGCTAAGCGAACTAGACAAAGCGGGGTTCATAAAACGACAACTAGTA
GAAACGCGACAAATAACGAAACACGTAGCGCAAATACTAGACAGCCGAATGAA
CACGAAATACGACGAAAACGACAAACTAATACGAGAAGTAAAAGTAATAACGC
TAAAAAGCAAACTAGTAAGCGACTTCCGAAAAGACTTCCAATTCTACAAAGTAC
GAGAAATAAACAACTACCACCACGCGCACGACGCGTACCTAAACGCGGTAGTAG
GGACGGCGCTAATAAAAAAATACCCGAAACTAGAAAGCGAATTCGTATACGGGG
ACTACAAAGTATACGACGTACGAAAAATGATAGCGAAAAGCGAACAAGAAATA
GGGAAAGCGACGGCGAAATACTTCTTCTACAGCAACATAATGAACTTCTTCAAA
ACGGAAATAACGCTAGCGAACGGGGAAATACGAAAACGACCGCTAATAGAAAC
GAACGGGGAAACGGGGGAAATAGTATGGGACAAAGGGCGAGACTTCGCGACGG
TACGAAAAGTACTAAGCATGCCGCAAGTAAACATAGTAAAAAAAACGGAAGTA
CAAACGGGGGGGTTCAGCAAAGAAAGCATACTACCGAAACGAAACAGCGACAA
ACTAATAGCGCGAAAAAAAGACTGGGACCCGAAAAAATACGGGGGGTTCGACA
GCCCGACGGTAGCGTACAGCGTACTAGTAGTAGCGAAAGTAGAAAAAGGGAAA
AGCAAAAAACTAAAAAGCGTAAAAGAACTACTAGGGATAACGATAATGGAACG
AAGCAGCTTCGAAAAAAACCCGATAGACTTCCTAGAAGCGAAAGGGTACAAAG
AAGTAAAAAAAGACCTAATAATAAAACTACCGAAATACAGCCTATTCGAACTAG
AAAACGGGCGAAAACGAATGCTAGCGAGCGCGGGGGAACTACAAAAAGGGAAC
GAACTAGCGCTACCGAGCAAATACGTAAACTTCCTATACCTAGCGAGCCACTAC
GAAAAACTAAAAGGGAGCCCGGAAGACAACGAACAAAAACAACTATTCGTAGA
ACAACACAAACACTACCTAGACGAAATAATAGAACAAATAAGCGAATTCAGCAA
ACGAGTAATACTAGCGGACGCGAACCTAGACAAAGTACTAAGCGCGTACAACAA
ACACCGAGACAAACCGATACGAGAACAAGCGGAAAACATAATACACCTATTCAC
GCTAACGAACCTAGGGGCGCCGGCGGCGTTCAAATACTTCGACACGACGATAGA
CCGAAAACGATACACGAGCACGAAAGAAGTACTAGACGCGACGCTAATACACC
AAAGCATAACGGGGCTATACGAAACGCGAATAGACCTAAGCCAACTAGGGGGG
GACGGGGGGGGGAGCCCGAAAAAAAAACGAAAAGTATGACTAGCCATCACATT
TAAAAGCATCTCAGCCTACCATGAGAATAAGAGAAAGAAAATGAAGATCAATAG
CTTATTCATCTCTTTTTCTTTTTCGTTGGTGTAAAGCCAACACCCTGTCTAAAAAA
CATAAATTTCTTTAATCATTTTGCCTCTTTTCTCTGTGCTTCAATTAATAAAAAAT
GGAAAGAACCTCGAG
SpCas9 mRNA #5
[SEQ ID NO: 1354]
AGGTCCCGCAGTCGGCGTCCAGCGGCTCTGCTTGTTCGTGTGTGTGTCGTTGCAG
GCCTTATTCGGATCCGCCACCATGGACAAGAAGTACAGCATCGGACTGGACATC
GGAACAAACAGCGTCGGATGGGCAGTCATCACAGACGAATACAAGGTCCCGAGC
AAGAAGTTCAAGGTCCTGGGAAACACAGACAGACACAGCATCAAGAAGAACCT
GATCGGAGCACTGCTGTTCGACAGCGGAGAAACAGCAGAAGCAACAAGACTGA
AGAGAACAGCAAGAAGAAGATACACAAGAAGAAAGAACAGAATCTGCTACCTG
CAGGAAATCTTCAGCAACGAAATGGCAAAGGTCGACGACAGCTTCTTCCACAGA
CTGGAAGAAAGCTTCCTGGTCGAAGAAGACAAGAAGCACGAAAGACACCCGAT
CTTCGGAAACATCGTCGACGAAGTCGCATACCACGAAAAGTACCCGACAATCTA
CCACCTGAGAAAGAAGCTGGTCGACAGCACAGACAAGGCAGACCTGAGACTGAT
CTACCTGGCACTGGCACACATGATCAAGTTCAGAGGACACTTCCTGATCGAAGG
AGACCTGAACCCGGACAACAGCGACGTCGACAAGCTGTTCATCCAGCTGGTCCA
GACATACAACCAGCTGTTCGAAGAAAACCCGATCAACGCAAGCGGAGTCGACGC
AAAGGCAATCCTGAGCGCAAGACTGAGCAAGAGCAGAAGACTGGAAAACCTGA
TCGCACAGCTGCCGGGAGAAAAGAAGAACGGACTGTTCGGAAACCTGATCGCAC
TGAGCCTGGGACTGACACCGAACTTCAAGAGCAACTTCGACCTGGCAGAAGACG
CAAAGCTGCAGCTGAGCAAGGACACATACGACGACGACCTGGACAACCTGCTGG
CACAGATCGGAGACCAGTACGCAGACCTGTTCCTGGCAGCAAAGAACCTGAGCG
ACGCAATCCTGCTGAGCGACATCCTGAGAGTCAACACAGAAATCACAAAGGCAC
CGCTGAGCGCAAGCATGATCAAGAGATACGACGAACACCACCAGGACCTGACAC
TGCTGAAGGCACTGGTCAGACAGCAGCTGCCGGAAAAGTACAAGGAAATCTTCT
TCGACCAGAGCAAGAACGGATACGCAGGATACATCGACGGAGGAGCAAGCCAG
GAAGAATTCTACAAGTTCATCAAGCCGATCCTGGAAAAGATGGACGGAACAGAA
GAACTGCTGGTCAAGCTGAACAGAGAAGACCTGCTGAGAAAGCAGAGAACATTC
GACAACGGAAGCATCCCGCACCAGATCCACCTGGGAGAACTGCACGCAATCCTG
AGAAGACAGGAAGACTTCTACCCGTTCCTGAAGGACAACAGAGAAAAGATCGA
AAAGATCCTGACATTCAGAATCCCGTACTACGTCGGACCGCTGGCAAGAGGAAA
CAGCAGATTCGCATGGATGACAAGAAAGAGCGAAGAAACAATCACACCGTGGA
ACTTCGAAGAAGTCGTCGACAAGGGAGCAAGCGCACAGAGCTTCATCGAAAGAA
TGACAAACTTCGACAAGAACCTGCCGAACGAAAAGGTCCTGCCGAAGCACAGCC
TGCTGTACGAATACTTCACAGTCTACAACGAACTGACAAAGGTCAAGTACGTCA
CAGAAGGAATGAGAAAGCCGGCATTCCTGAGCGGAGAACAGAAGAAGGCAATC
GTCGACCTGCTGTTCAAGACAAACAGAAAGGTCACAGTCAAGCAGCTGAAGGAA
GACTACTTCAAGAAGATCGAATGCTTCGACAGCGTCGAAATCAGCGGAGTCGAA
GACAGATTCAACGCAAGCCTGGGAACATACCACGACCTGCTGAAGATCATCAAG
GACAAGGACTTCCTGGACAACGAAGAAAACGAAGACATCCTGGAAGACATCGTC
CTGACACTGACACTGTTCGAAGACAGAGAAATGATCGAAGAAAGACTGAAGACA
TACGCACACCTGTTCGACGACAAGGTCATGAAGCAGCTGAAGAGAAGAAGATAC
ACAGGATGGGGAAGACTGAGCAGAAAGCTGATCAACGGAATCAGAGACAAGCA
GAGCGGAAAGACAATCCTGGACTTCCTGAAGAGCGACGGATTCGCAAACAGAAA
CTTCATGCAGCTGATCCACGACGACAGCCTGACATTCAAGGAAGACATCCAGAA
GGCACAGGTCAGCGGACAGGGAGACAGCCTGCACGAACACATCGCAAACCTGG
CAGGAAGCCCGGCAATCAAGAAGGGAATCCTGCAGACAGTCAAGGTCGTCGACG
AACTGGTCAAGGTCATGGGAAGACACAAGCCGGAAAACATCGTCATCGAAATGG
CAAGAGAAAACCAGACAACACAGAAGGGACAGAAGAACAGCAGAGAAAGAAT
GAAGAGAATCGAAGAAGGAATCAAGGAACTGGGAAGCCAGATCCTGAAGGAAC
ACCCGGTCGAAAACACACAGCTGCAGAACGAAAAGCTGTACCTGTACTACCTGC
AGAACGGAAGAGACATGTACGTCGACCAGGAACTGGACATCAACAGACTGAGC
GACTACGACGTCGACCACATCGTCCCGCAGAGCTTCCTGAAGGACGACAGCATC
GACAACAAGGTCCTGACAAGAAGCGACAAGAACAGAGGAAAGAGCGACAACGT
CCCGAGCGAAGAAGTCGTCAAGAAGATGAAGAACTACTGGAGACAGCTGCTGA
ACGCAAAGCTGATCACACAGAGAAAGTTCGACAACCTGACAAAGGCAGAGAGA
GGAGGACTGAGCGAACTGGACAAGGCAGGATTCATCAAGAGACAGCTGGTCGA
AACAAGACAGATCACAAAGCACGTCGCACAGATCCTGGACAGCAGAATGAACA
CAAAGTACGACGAAAACGACAAGCTGATCAGAGAAGTCAAGGTCATCACACTGA
AGAGCAAGCTGGTCAGCGACTTCAGAAAGGACTTCCAGTTCTACAAGGTCAGAG
AAATCAACAACTACCACCACGCACACGACGCATACCTGAACGCAGTCGTCGGAA
CAGCACTGATCAAGAAGTACCCGAAGCTGGAAAGCGAATTCGTCTACGGAGACT
ACAAGGTCTACGACGTCAGAAAGATGATCGCAAAGAGCGAACAGGAAATCGGA
AAGGCAACAGCAAAGTACTTCTTCTACAGCAACATCATGAACTTCTTCAAGACA
GAAATCACACTGGCAAACGGAGAAATCAGAAAGAGACCGCTGATCGAAACAAA
CGGAGAAACAGGAGAAATCGTCTGGGACAAGGGAAGAGACTTCGCAACAGTCA
GAAAGGTCCTGAGCATGCCGCAGGTCAACATCGTCAAGAAGACAGAAGTCCAGA
CAGGAGGATTCAGCAAGGAAAGCATCCTGCCGAAGAGAAACAGCGACAAGCTG
ATCGCAAGAAAGAAGGACTGGGACCCGAAGAAGTACGGAGGATTCGACAGCCC
GACAGTCGCATACAGCGTCCTGGTCGTCGCAAAGGTCGAAAAGGGAAAGAGCAA
GAAGCTGAAGAGCGTCAAGGAACTGCTGGGAATCACAATCATGGAAAGAAGCA
GCTTCGAAAAGAACCCGATCGACTTCCTGGAAGCAAAGGGATACAAGGAAGTCA
AGAAGGACCTGATCATCAAGCTGCCGAAGTACAGCCTGTTCGAACTGGAAAACG
GAAGAAAGAGAATGCTGGCAAGCGCAGGAGAACTGCAGAAGGGAAACGAACTG
GCACTGCCGAGCAAGTACGTCAACTTCCTGTACCTGGCAAGCCACTACGAAAAG
CTGAAGGGAAGCCCGGAAGACAACGAACAGAAGCAGCTGTTCGTCGAACAGCA
CAAGCACTACCTGGACGAAATCATCGAACAGATCAGCGAATTCAGCAAGAGAGT
CATCCTGGCAGACGCAAACCTGGACAAGGTCCTGAGCGCATACAACAAGCACAG
AGACAAGCCGATCAGAGAACAGGCAGAAAACATCATCCACCTGTTCACACTGAC
AAACCTGGGAGCACCGGCAGCATTCAAGTACTTCGACACAACAATCGACAGAAA
GAGATACACAAGCACAAAGGAAGTCCTGGACGCAACACTGATCCACCAGAGCAT
CACAGGACTGTACGAAACAAGAATCGACCTGAGCCAGCTGGGAGGAGACGGAG
GAGGAAGCCCGAAGAAGAAGAGAAAGGTCTAGCTAGCCATCACATTTAAAAGC
ATCTCAGCCTACCATGAGAATAAGAGAAAGAAAATGAAGATCAATAGCTTATTC
ATCTCTTTTTCTTTTTCGTTGGTGTAAAGCCAACACCCTGTCTAAAAAACATAAAT
TTCTTTAATCATTTTGCCTCTTTTCTCTGTGCTTCAATTAATAAAAAATGGAAAGA
ACCTCGAG
SpCas9 mRNA #6
[SEQ ID NO: 1355]
GGGCAGATCGCCTGGAGACGCCATCCACGCTGTTTTGACCTCCATAGAAGACAC
CGGGACCGATCCAGCCTCCGCGGCCGGGAACGGTGCATTGGAACGCGGATTCCC
CGTGCCAAGAGTGACTCACCGTCCTTGACACGGCCACCATGGACAAGAAGTACA
GCATCGGACTGGACATCGGAACAAACAGCGTCGGATGGGCAGTCATCACAGACG
AATACAAGGTCCCGAGCAAGAAGTTCAAGGTCCTGGGAAACACAGACAGACAC
AGCATCAAGAAGAACCTGATCGGAGCACTGCTGTTCGACAGCGGAGAAACAGCA
GAAGCAACAAGACTGAAGAGAACAGCAAGAAGAAGATACACAAGAAGAAAGA
ACAGAATCTGCTACCTGCAGGAAATCTTCAGCAACGAAATGGCAAAGGTCGACG
ACAGCTTCTTCCACAGACTGGAAGAAAGCTTCCTGGTCGAAGAAGACAAGAAGC
ACGAAAGACACCCGATCTTCGGAAACATCGTCGACGAAGTCGCATACCACGAAA
AGTACCCGACAATCTACCACCTGAGAAAGAAGCTGGTCGACAGCACAGACAAGG
CAGACCTGAGACTGATCTACCTGGCACTGGCACACATGATCAAGTTCAGAGGAC
ACTTCCTGATCGAAGGAGACCTGAACCCGGACAACAGCGACGTCGACAAGCTGT
TCATCCAGCTGGTCCAGACATACAACCAGCTGTTCGAAGAAAACCCGATCAACG
CAAGCGGAGTCGACGCAAAGGCAATCCTGAGCGCAAGACTGAGCAAGAGCAGA
AGACTGGAAAACCTGATCGCACAGCTGCCGGGAGAAAAGAAGAACGGACTGTTC
GGAAACCTGATCGCACTGAGCCTGGGACTGACACCGAACTTCAAGAGCAACTTC
GACCTGGCAGAAGACGCAAAGCTGCAGCTGAGCAAGGACACATACGACGACGA
CCTGGACAACCTGCTGGCACAGATCGGAGACCAGTACGCAGACCTGTTCCTGGC
AGCAAAGAACCTGAGCGACGCAATCCTGCTGAGCGACATCCTGAGAGTCAACAC
AGAAATCACAAAGGCACCGCTGAGCGCAAGCATGATCAAGAGATACGACGAAC
ACCACCAGGACCTGACACTGCTGAAGGCACTGGTCAGACAGCAGCTGCCGGAAA
AGTACAAGGAAATCTTCTTCGACCAGAGCAAGAACGGATACGCAGGATACATCG
ACGGAGGAGCAAGCCAGGAAGAATTCTACAAGTTCATCAAGCCGATCCTGGAAA
AGATGGACGGAACAGAAGAACTGCTGGTCAAGCTGAACAGAGAAGACCTGCTG
AGAAAGCAGAGAACATTCGACAACGGAAGCATCCCGCACCAGATCCACCTGGGA
GAACTGCACGCAATCCTGAGAAGACAGGAAGACTTCTACCCGTTCCTGAAGGAC
AACAGAGAAAAGATCGAAAAGATCCTGACATTCAGAATCCCGTACTACGTCGGA
CCGCTGGCAAGAGGAAACAGCAGATTCGCATGGATGACAAGAAAGAGCGAAGA
AACAATCACACCGTGGAACTTCGAAGAAGTCGTCGACAAGGGAGCAAGCGCACA
GAGCTTCATCGAAAGAATGACAAACTTCGACAAGAACCTGCCGAACGAAAAGGT
CCTGCCGAAGCACAGCCTGCTGTACGAATACTTCACAGTCTACAACGAACTGAC
AAAGGTCAAGTACGTCACAGAAGGAATGAGAAAGCCGGCATTCCTGAGCGGAG
AACAGAAGAAGGCAATCGTCGACCTGCTGTTCAAGACAAACAGAAAGGTCACAG
TCAAGCAGCTGAAGGAAGACTACTTCAAGAAGATCGAATGCTTCGACAGCGTCG
AAATCAGCGGAGTCGAAGACAGATTCAACGCAAGCCTGGGAACATACCACGACC
TGCTGAAGATCATCAAGGACAAGGACTTCCTGGACAACGAAGAAAACGAAGAC
ATCCTGGAAGACATCGTCCTGACACTGACACTGTTCGAAGACAGAGAAATGATC
GAAGAAAGACTGAAGACATACGCACACCTGTTCGACGACAAGGTCATGAAGCAG
CTGAAGAGAAGAAGATACACAGGATGGGGAAGACTGAGCAGAAAGCTGATCAA
CGGAATCAGAGACAAGCAGAGCGGAAAGACAATCCTGGACTTCCTGAAGAGCG
ACGGATTCGCAAACAGAAACTTCATGCAGCTGATCCACGACGACAGCCTGACAT
TCAAGGAAGACATCCAGAAGGCACAGGTCAGCGGACAGGGAGACAGCCTGCAC
GAACACATCGCAAACCTGGCAGGAAGCCCGGCAATCAAGAAGGGAATCCTGCA
GACAGTCAAGGTCGTCGACGAACTGGTCAAGGTCATGGGAAGACACAAGCCGGA
AAACATCGTCATCGAAATGGCAAGAGAAAACCAGACAACACAGAAGGGACAGA
AGAACAGCAGAGAAAGAATGAAGAGAATCGAAGAAGGAATCAAGGAACTGGGA
AGCCAGATCCTGAAGGAACACCCGGTCGAAAACACACAGCTGCAGAACGAAAA
GCTGTACCTGTACTACCTGCAGAACGGAAGAGACATGTACGTCGACCAGGAACT
GGACATCAACAGACTGAGCGACTACGACGTCGACCACATCGTCCCGCAGAGCTT
CCTGAAGGACGACAGCATCGACAACAAGGTCCTGACAAGAAGCGACAAGAACA
GAGGAAAGAGCGACAACGTCCCGAGCGAAGAAGTCGTCAAGAAGATGAAGAAC
TACTGGAGACAGCTGCTGAACGCAAAGCTGATCACACAGAGAAAGTTCGACAAC
CTGACAAAGGCAGAGAGAGGAGGACTGAGCGAACTGGACAAGGCAGGATTCAT
CAAGAGACAGCTGGTCGAAACAAGACAGATCACAAAGCACGTCGCACAGATCCT
GGACAGCAGAATGAACACAAAGTACGACGAAAACGACAAGCTGATCAGAGAAG
TCAAGGTCATCACACTGAAGAGCAAGCTGGTCAGCGACTTCAGAAAGGACTTCC
AGTTCTACAAGGTCAGAGAAATCAACAACTACCACCACGCACACGACGCATACC
TGAACGCAGTCGTCGGAACAGCACTGATCAAGAAGTACCCGAAGCTGGAAAGCG
AATTCGTCTACGGAGACTACAAGGTCTACGACGTCAGAAAGATGATCGCAAAGA
GCGAACAGGAAATCGGAAAGGCAACAGCAAAGTACTTCTTCTACAGCAACATCA
TGAACTTCTTCAAGACAGAAATCACACTGGCAAACGGAGAAATCAGAAAGAGAC
CGCTGATCGAAACAAACGGAGAAACAGGAGAAATCGTCTGGGACAAGGGAAGA
GACTTCGCAACAGTCAGAAAGGTCCTGAGCATGCCGCAGGTCAACATCGTCAAG
AAGACAGAAGTCCAGACAGGAGGATTCAGCAAGGAAAGCATCCTGCCGAAGAG
AAACAGCGACAAGCTGATCGCAAGAAAGAAGGACTGGGACCCGAAGAAGTACG
GAGGATTCGACAGCCCGACAGTCGCATACAGCGTCCTGGTCGTCGCAAAGGTCG
AAAAGGGAAAGAGCAAGAAGCTGAAGAGCGTCAAGGAACTGCTGGGAATCACA
ATCATGGAAAGAAGCAGCTTCGAAAAGAACCCGATCGACTTCCTGGAAGCAAAG
GGATACAAGGAAGTCAAGAAGGACCTGATCATCAAGCTGCCGAAGTACAGCCTG
TTCGAACTGGAAAACGGAAGAAAGAGAATGCTGGCAAGCGCAGGAGAACTGCA
GAAGGGAAACGAACTGGCACTGCCGAGCAAGTACGTCAACTTCCTGTACCTGGC
AAGCCACTACGAAAAGCTGAAGGGAAGCCCGGAAGACAACGAACAGAAGCAGC
TGTTCGTCGAACAGCACAAGCACTACCTGGACGAAATCATCGAACAGATCAGCG
AATTCAGCAAGAGAGTCATCCTGGCAGACGCAAACCTGGACAAGGTCCTGAGCG
CATACAACAAGCACAGAGACAAGCCGATCAGAGAACAGGCAGAAAACATCATC
CACCTGTTCACACTGACAAACCTGGGAGCACCGGCAGCATTCAAGTACTTCGAC
ACAACAATCGACAGAAAGAGATACACAAGCACAAAGGAAGTCCTGGACGCAAC
ACTGATCCACCAGAGCATCACAGGACTGTACGAAACAAGAATCGACCTGAGCCA
GCTGGGAGGAGACGGAGGAGGAAGCCCGAAGAAGAAGAGAAAGGTCTAGCTAG
CCATCACATTTAAAAGCATCTCAGCCTACCATGAGAATAAGAGAAAGAAAATGA
AGATCAATAGCTTATTCATCTCTTTTTCTTTTTCGTTGGTGTAAAGCCAACACCCT
GTCTAAAAAACATAAATTTCTTTAATCATTTTGCCTCTTTTCTCTGTGCTTCAATT
AATAAAAAATGGAAAGAACCTCGAG
SpCas9 mRNA #7
[SEQ ID NO: 1356]
GGGACATTTGCTTCTGACACAACTGTGTTCACTAGCAACCTCAAACAGACACCGG
ATCTGCCACCATGGACAAGAAGTACAGCATCGGACTGGACATCGGAACAAACAG
CGTCGGATGGGCAGTCATCACAGACGAATACAAGGTCCCGAGCAAGAAGTTCAA
GGTCCTGGGAAACACAGACAGACACAGCATCAAGAAGAACCTGATCGGAGCACT
GCTGTTCGACAGCGGAGAAACAGCAGAAGCAACAAGACTGAAGAGAACAGCAA
GAAGAAGATACACAAGAAGAAAGAACAGAATCTGCTACCTGCAGGAAATCTTCA
GCAACGAAATGGCAAAGGTCGACGACAGCTTCTTCCACAGACTGGAAGAAAGCT
TCCTGGTCGAAGAAGACAAGAAGCACGAAAGACACCCGATCTTCGGAAACATCG
TCGACGAAGTCGCATACCACGAAAAGTACCCGACAATCTACCACCTGAGAAAGA
AGCTGGTCGACAGCACAGACAAGGCAGACCTGAGACTGATCTACCTGGCACTGG
CACACATGATCAAGTTCAGAGGACACTTCCTGATCGAAGGAGACCTGAACCCGG
ACAACAGCGACGTCGACAAGCTGTTCATCCAGCTGGTCCAGACATACAACCAGC
TGTTCGAAGAAAACCCGATCAACGCAAGCGGAGTCGACGCAAAGGCAATCCTGA
GCGCAAGACTGAGCAAGAGCAGAAGACTGGAAAACCTGATCGCACAGCTGCCG
GGAGAAAAGAAGAACGGACTGTTCGGAAACCTGATCGCACTGAGCCTGGGACTG
ACACCGAACTTCAAGAGCAACTTCGACCTGGCAGAAGACGCAAAGCTGCAGCTG
AGCAAGGACACATACGACGACGACCTGGACAACCTGCTGGCACAGATCGGAGAC
CAGTACGCAGACCTGTTCCTGGCAGCAAAGAACCTGAGCGACGCAATCCTGCTG
AGCGACATCCTGAGAGTCAACACAGAAATCACAAAGGCACCGCTGAGCGCAAGC
ATGATCAAGAGATACGACGAACACCACCAGGACCTGACACTGCTGAAGGCACTG
GTCAGACAGCAGCTGCCGGAAAAGTACAAGGAAATCTTCTTCGACCAGAGCAAG
AACGGATACGCAGGATACATCGACGGAGGAGCAAGCCAGGAAGAATTCTACAA
GTTCATCAAGCCGATCCTGGAAAAGATGGACGGAACAGAAGAACTGCTGGTCAA
GCTGAACAGAGAAGACCTGCTGAGAAAGCAGAGAACATTCGACAACGGAAGCA
TCCCGCACCAGATCCACCTGGGAGAACTGCACGCAATCCTGAGAAGACAGGAAG
ACTTCTACCCGTTCCTGAAGGACAACAGAGAAAAGATCGAAAAGATCCTGACAT
TCAGAATCCCGTACTACGTCGGACCGCTGGCAAGAGGAAACAGCAGATTCGCAT
GGATGACAAGAAAGAGCGAAGAAACAATCACACCGTGGAACTTCGAAGAAGTC
GTCGACAAGGGAGCAAGCGCACAGAGCTTCATCGAAAGAATGACAAACTTCGAC
AAGAACCTGCCGAACGAAAAGGTCCTGCCGAAGCACAGCCTGCTGTACGAATAC
TTCACAGTCTACAACGAACTGACAAAGGTCAAGTACGTCACAGAAGGAATGAGA
AAGCCGGCATTCCTGAGCGGAGAACAGAAGAAGGCAATCGTCGACCTGCTGTTC
AAGACAAACAGAAAGGTCACAGTCAAGCAGCTGAAGGAAGACTACTTCAAGAA
GATCGAATGCTTCGACAGCGTCGAAATCAGCGGAGTCGAAGACAGATTCAACGC
AAGCCTGGGAACATACCACGACCTGCTGAAGATCATCAAGGACAAGGACTTCCT
GGACAACGAAGAAAACGAAGACATCCTGGAAGACATCGTCCTGACACTGACACT
GTTCGAAGACAGAGAAATGATCGAAGAAAGACTGAAGACATACGCACACCTGTT
CGACGACAAGGTCATGAAGCAGCTGAAGAGAAGAAGATACACAGGATGGGGAA
GACTGAGCAGAAAGCTGATCAACGGAATCAGAGACAAGCAGAGCGGAAAGACA
ATCCTGGACTTCCTGAAGAGCGACGGATTCGCAAACAGAAACTTCATGCAGCTG
ATCCACGACGACAGCCTGACATTCAAGGAAGACATCCAGAAGGCACAGGTCAGC
GGACAGGGAGACAGCCTGCACGAACACATCGCAAACCTGGCAGGAAGCCCGGC
AATCAAGAAGGGAATCCTGCAGACAGTCAAGGTCGTCGACGAACTGGTCAAGGT
CATGGGAAGACACAAGCCGGAAAACATCGTCATCGAAATGGCAAGAGAAAACC
AGACAACACAGAAGGGACAGAAGAACAGCAGAGAAAGAATGAAGAGAATCGA
AGAAGGAATCAAGGAACTGGGAAGCCAGATCCTGAAGGAACACCCGGTCGAAA
ACACACAGCTGCAGAACGAAAAGCTGTACCTGTACTACCTGCAGAACGGAAGAG
ACATGTACGTCGACCAGGAACTGGACATCAACAGACTGAGCGACTACGACGTCG
ACCACATCGTCCCGCAGAGCTTCCTGAAGGACGACAGCATCGACAACAAGGTCC
TGACAAGAAGCGACAAGAACAGAGGAAAGAGCGACAACGTCCCGAGCGAAGAA
GTCGTCAAGAAGATGAAGAACTACTGGAGACAGCTGCTGAACGCAAAGCTGATC
ACACAGAGAAAGTTCGACAACCTGACAAAGGCAGAGAGAGGAGGACTGAGCGA
ACTGGACAAGGCAGGATTCATCAAGAGACAGCTGGTCGAAACAAGACAGATCAC
AAAGCACGTCGCACAGATCCTGGACAGCAGAATGAACACAAAGTACGACGAAA
ACGACAAGCTGATCAGAGAAGTCAAGGTCATCACACTGAAGAGCAAGCTGGTCA
GCGACTTCAGAAAGGACTTCCAGTTCTACAAGGTCAGAGAAATCAACAACTACC
ACCACGCACACGACGCATACCTGAACGCAGTCGTCGGAACAGCACTGATCAAGA
AGTACCCGAAGCTGGAAAGCGAATTCGTCTACGGAGACTACAAGGTCTACGACG
TCAGAAAGATGATCGCAAAGAGCGAACAGGAAATCGGAAAGGCAACAGCAAAG
TACTTCTTCTACAGCAACATCATGAACTTCTTCAAGACAGAAATCACACTGGCAA
ACGGAGAAATCAGAAAGAGACCGCTGATCGAAACAAACGGAGAAACAGGAGAA
ATCGTCTGGGACAAGGGAAGAGACTTCGCAACAGTCAGAAAGGTCCTGAGCATG
CCGCAGGTCAACATCGTCAAGAAGACAGAAGTCCAGACAGGAGGATTCAGCAA
GGAAAGCATCCTGCCGAAGAGAAACAGCGACAAGCTGATCGCAAGAAAGAAGG
ACTGGGACCCGAAGAAGTACGGAGGATTCGACAGCCCGACAGTCGCATACAGCG
TCCTGGTCGTCGCAAAGGTCGAAAAGGGAAAGAGCAAGAAGCTGAAGAGCGTC
AAGGAACTGCTGGGAATCACAATCATGGAAAGAAGCAGCTTCGAAAAGAACCC
GATCGACTTCCTGGAAGCAAAGGGATACAAGGAAGTCAAGAAGGACCTGATCAT
CAAGCTGCCGAAGTACAGCCTGTTCGAACTGGAAAACGGAAGAAAGAGAATGCT
GGCAAGCGCAGGAGAACTGCAGAAGGGAAACGAACTGGCACTGCCGAGCAAGT
ACGTCAACTTCCTGTACCTGGCAAGCCACTACGAAAAGCTGAAGGGAAGCCCGG
AAGACAACGAACAGAAGCAGCTGTTCGTCGAACAGCACAAGCACTACCTGGACG
AAATCATCGAACAGATCAGCGAATTCAGCAAGAGAGTCATCCTGGCAGACGCAA
ACCTGGACAAGGTCCTGAGCGCATACAACAAGCACAGAGACAAGCCGATCAGA
GAACAGGCAGAAAACATCATCCACCTGTTCACACTGACAAACCTGGGAGCACCG
GCAGCATTCAAGTACTTCGACACAACAATCGACAGAAAGAGATACACAAGCACA
AAGGAAGTCCTGGACGCAACACTGATCCACCAGAGCATCACAGGACTGTACGAA
ACAAGAATCGACCTGAGCCAGCTGGGAGGAGACGGAGGAGGAAGCCCGAAGAA
GAAGAGAAAGGTCTAGCTAGCGCTCGCTTTCTTGCTGTCCAATTTCTATTAAAGG
TTCCTTTGTTCCCTAAGTCCAACTACTAAACTGGGGGATATTATGAAGGGCCTTG
AGCATCTGGATTCTGCCTAATAAAAAACATTTATTTTCATTGCCTCGAG
SpCas9 mRNA #8
[SEQ ID NO: 1357]
GGGAAGCTCAGAATAAACGCTCAACTTTGGCCGGATCTGCCACCATGGACAAGA
AGTACAGCATCGGACTGGACATCGGAACAAACAGCGTCGGATGGGCAGTCATCA
CAGACGAATACAAGGTCCCGAGCAAGAAGTTCAAGGTCCTGGGAAACACAGAC
AGACACAGCATCAAGAAGAACCTGATCGGAGCACTGCTGTTCGACAGCGGAGAA
ACAGCAGAAGCAACAAGACTGAAGAGAACAGCAAGAAGAAGATACACAAGAAG
AAAGAACAGAATCTGCTACCTGCAGGAAATCTTCAGCAACGAAATGGCAAAGGT
CGACGACAGCTTCTTCCACAGACTGGAAGAAAGCTTCCTGGTCGAAGAAGACAA
GAAGCACGAAAGACACCCGATCTTCGGAAACATCGTCGACGAAGTCGCATACCA
CGAAAAGTACCCGACAATCTACCACCTGAGAAAGAAGCTGGTCGACAGCACAGA
CAAGGCAGACCTGAGACTGATCTACCTGGCACTGGCACACATGATCAAGTTCAG
AGGACACTTCCTGATCGAAGGAGACCTGAACCCGGACAACAGCGACGTCGACAA
GCTGTTCATCCAGCTGGTCCAGACATACAACCAGCTGTTCGAAGAAAACCCGATC
AACGCAAGCGGAGTCGACGCAAAGGCAATCCTGAGCGCAAGACTGAGCAAGAG
CAGAAGACTGGAAAACCTGATCGCACAGCTGCCGGGAGAAAAGAAGAACGGAC
TGTTCGGAAACCTGATCGCACTGAGCCTGGGACTGACACCGAACTTCAAGAGCA
ACTTCGACCTGGCAGAAGACGCAAAGCTGCAGCTGAGCAAGGACACATACGACG
ACGACCTGGACAACCTGCTGGCACAGATCGGAGACCAGTACGCAGACCTGTTCC
TGGCAGCAAAGAACCTGAGCGACGCAATCCTGCTGAGCGACATCCTGAGAGTCA
ACACAGAAATCACAAAGGCACCGCTGAGCGCAAGCATGATCAAGAGATACGAC
GAACACCACCAGGACCTGACACTGCTGAAGGCACTGGTCAGACAGCAGCTGCCG
GAAAAGTACAAGGAAATCTTCTTCGACCAGAGCAAGAACGGATACGCAGGATAC
ATCGACGGAGGAGCAAGCCAGGAAGAATTCTACAAGTTCATCAAGCCGATCCTG
GAAAAGATGGACGGAACAGAAGAACTGCTGGTCAAGCTGAACAGAGAAGACCT
GCTGAGAAAGCAGAGAACATTCGACAACGGAAGCATCCCGCACCAGATCCACCT
GGGAGAACTGCACGCAATCCTGAGAAGACAGGAAGACTTCTACCCGTTCCTGAA
GGACAACAGAGAAAAGATCGAAAAGATCCTGACATTCAGAATCCCGTACTACGT
CGGACCGCTGGCAAGAGGAAACAGCAGATTCGCATGGATGACAAGAAAGAGCG
AAGAAACAATCACACCGTGGAACTTCGAAGAAGTCGTCGACAAGGGAGCAAGC
GCACAGAGCTTCATCGAAAGAATGACAAACTTCGACAAGAACCTGCCGAACGAA
AAGGTCCTGCCGAAGCACAGCCTGCTGTACGAATACTTCACAGTCTACAACGAA
CTGACAAAGGTCAAGTACGTCACAGAAGGAATGAGAAAGCCGGCATTCCTGAGC
GGAGAACAGAAGAAGGCAATCGTCGACCTGCTGTTCAAGACAAACAGAAAGGT
CACAGTCAAGCAGCTGAAGGAAGACTACTTCAAGAAGATCGAATGCTTCGACAG
CGTCGAAATCAGCGGAGTCGAAGACAGATTCAACGCAAGCCTGGGAACATACCA
CGACCTGCTGAAGATCATCAAGGACAAGGACTTCCTGGACAACGAAGAAAACGA
AGACATCCTGGAAGACATCGTCCTGACACTGACACTGTTCGAAGACAGAGAAAT
GATCGAAGAAAGACTGAAGACATACGCACACCTGTTCGACGACAAGGTCATGAA
GCAGCTGAAGAGAAGAAGATACACAGGATGGGGAAGACTGAGCAGAAAGCTGA
TCAACGGAATCAGAGACAAGCAGAGCGGAAAGACAATCCTGGACTTCCTGAAGA
GCGACGGATTCGCAAACAGAAACTTCATGCAGCTGATCCACGACGACAGCCTGA
CATTCAAGGAAGACATCCAGAAGGCACAGGTCAGCGGACAGGGAGACAGCCTG
CACGAACACATCGCAAACCTGGCAGGAAGCCCGGCAATCAAGAAGGGAATCCTG
CAGACAGTCAAGGTCGTCGACGAACTGGTCAAGGTCATGGGAAGACACAAGCCG
GAAAACATCGTCATCGAAATGGCAAGAGAAAACCAGACAACACAGAAGGGACA
GAAGAACAGCAGAGAAAGAATGAAGAGAATCGAAGAAGGAATCAAGGAACTGG
GAAGCCAGATCCTGAAGGAACACCCGGTCGAAAACACACAGCTGCAGAACGAA
AAGCTGTACCTGTACTACCTGCAGAACGGAAGAGACATGTACGTCGACCAGGAA
CTGGACATCAACAGACTGAGCGACTACGACGTCGACCACATCGTCCCGCAGAGC
TTCCTGAAGGACGACAGCATCGACAACAAGGTCCTGACAAGAAGCGACAAGAAC
AGAGGAAAGAGCGACAACGTCCCGAGCGAAGAAGTCGTCAAGAAGATGAAGAA
CTACTGGAGACAGCTGCTGAACGCAAAGCTGATCACACAGAGAAAGTTCGACAA
CCTGACAAAGGCAGAGAGAGGAGGACTGAGCGAACTGGACAAGGCAGGATTCA
TCAAGAGACAGCTGGTCGAAACAAGACAGATCACAAAGCACGTCGCACAGATCC
TGGACAGCAGAATGAACACAAAGTACGACGAAAACGACAAGCTGATCAGAGAA
GTCAAGGTCATCACACTGAAGAGCAAGCTGGTCAGCGACTTCAGAAAGGACTTC
CAGTTCTACAAGGTCAGAGAAATCAACAACTACCACCACGCACACGACGCATAC
CTGAACGCAGTCGTCGGAACAGCACTGATCAAGAAGTACCCGAAGCTGGAAAGC
GAATTCGTCTACGGAGACTACAAGGTCTACGACGTCAGAAAGATGATCGCAAAG
AGCGAACAGGAAATCGGAAAGGCAACAGCAAAGTACTTCTTCTACAGCAACATC
ATGAACTTCTTCAAGACAGAAATCACACTGGCAAACGGAGAAATCAGAAAGAGA
CCGCTGATCGAAACAAACGGAGAAACAGGAGAAATCGTCTGGGACAAGGGAAG
AGACTTCGCAACAGTCAGAAAGGTCCTGAGCATGCCGCAGGTCAACATCGTCAA
GAAGACAGAAGTCCAGACAGGAGGATTCAGCAAGGAAAGCATCCTGCCGAAGA
GAAACAGCGACAAGCTGATCGCAAGAAAGAAGGACTGGGACCCGAAGAAGTAC
GGAGGATTCGACAGCCCGACAGTCGCATACAGCGTCCTGGTCGTCGCAAAGGTC
GAAAAGGGAAAGAGCAAGAAGCTGAAGAGCGTCAAGGAACTGCTGGGAATCAC
AATCATGGAAAGAAGCAGCTTCGAAAAGAACCCGATCGACTTCCTGGAAGCAAA
GGGATACAAGGAAGTCAAGAAGGACCTGATCATCAAGCTGCCGAAGTACAGCCT
GTTCGAACTGGAAAACGGAAGAAAGAGAATGCTGGCAAGCGCAGGAGAACTGC
AGAAGGGAAACGAACTGGCACTGCCGAGCAAGTACGTCAACTTCCTGTACCTGG
CAAGCCACTACGAAAAGCTGAAGGGAAGCCCGGAAGACAACGAACAGAAGCAG
CTGTTCGTCGAACAGCACAAGCACTACCTGGACGAAATCATCGAACAGATCAGC
GAATTCAGCAAGAGAGTCATCCTGGCAGACGCAAACCTGGACAAGGTCCTGAGC
GCATACAACAAGCACAGAGACAAGCCGATCAGAGAACAGGCAGAAAACATCAT
CCACCTGTTCACACTGACAAACCTGGGAGCACCGGCAGCATTCAAGTACTTCGAC
ACAACAATCGACAGAAAGAGATACACAAGCACAAAGGAAGTCCTGGACGCAAC
ACTGATCCACCAGAGCATCACAGGACTGTACGAAACAAGAATCGACCTGAGCCA
GCTGGGAGGAGACGGAGGAGGAAGCCCGAAGAAGAAGAGAAAGGTCTAGCTAG
CACCAGCCTCAAGAACACCCGAATGGAGTCTCTAAGCTACATAATACCAACTTA
CACTTTACAAAATGTTGTCCCCCAAAATGTAGCCATTCGTATCTGCTCCTAATAA
AAAGAAAGTTTCTTCACATTCTCTCGAG
SpCas9 mRNA #9
[SEQ ID NO: 1358]
AGGAAGCTCAGAATAAACGCTCAACTTTGGCCGGATCTGCCACCATGGACAAGA
AGTACAGCATCGGACTGGACATCGGAACAAACAGCGTCGGATGGGCAGTCATCA
CAGACGAATACAAGGTCCCGAGCAAGAAGTTCAAGGTCCTGGGAAACACAGAC
AGACACAGCATCAAGAAGAACCTGATCGGAGCACTGCTGTTCGACAGCGGAGAA
ACAGCAGAAGCAACAAGACTGAAGAGAACAGCAAGAAGAAGATACACAAGAAG
AAAGAACAGAATCTGCTACCTGCAGGAAATCTTCAGCAACGAAATGGCAAAGGT
CGACGACAGCTTCTTCCACAGACTGGAAGAAAGCTTCCTGGTCGAAGAAGACAA
GAAGCACGAAAGACACCCGATCTTCGGAAACATCGTCGACGAAGTCGCATACCA
CGAAAAGTACCCGACAATCTACCACCTGAGAAAGAAGCTGGTCGACAGCACAGA
CAAGGCAGACCTGAGACTGATCTACCTGGCACTGGCACACATGATCAAGTTCAG
AGGACACTTCCTGATCGAAGGAGACCTGAACCCGGACAACAGCGACGTCGACAA
GCTGTTCATCCAGCTGGTCCAGACATACAACCAGCTGTTCGAAGAAAACCCGATC
AACGCAAGCGGAGTCGACGCAAAGGCAATCCTGAGCGCAAGACTGAGCAAGAG
CAGAAGACTGGAAAACCTGATCGCACAGCTGCCGGGAGAAAAGAAGAACGGAC
TGTTCGGAAACCTGATCGCACTGAGCCTGGGACTGACACCGAACTTCAAGAGCA
ACTTCGACCTGGCAGAAGACGCAAAGCTGCAGCTGAGCAAGGACACATACGACG
ACGACCTGGACAACCTGCTGGCACAGATCGGAGACCAGTACGCAGACCTGTTCC
TGGCAGCAAAGAACCTGAGCGACGCAATCCTGCTGAGCGACATCCTGAGAGTCA
ACACAGAAATCACAAAGGCACCGCTGAGCGCAAGCATGATCAAGAGATACGAC
GAACACCACCAGGACCTGACACTGCTGAAGGCACTGGTCAGACAGCAGCTGCCG
GAAAAGTACAAGGAAATCTTCTTCGACCAGAGCAAGAACGGATACGCAGGATAC
ATCGACGGAGGAGCAAGCCAGGAAGAATTCTACAAGTTCATCAAGCCGATCCTG
GAAAAGATGGACGGAACAGAAGAACTGCTGGTCAAGCTGAACAGAGAAGACCT
GCTGAGAAAGCAGAGAACATTCGACAACGGAAGCATCCCGCACCAGATCCACCT
GGGAGAACTGCACGCAATCCTGAGAAGACAGGAAGACTTCTACCCGTTCCTGAA
GGACAACAGAGAAAAGATCGAAAAGATCCTGACATTCAGAATCCCGTACTACGT
CGGACCGCTGGCAAGAGGAAACAGCAGATTCGCATGGATGACAAGAAAGAGCG
AAGAAACAATCACACCGTGGAACTTCGAAGAAGTCGTCGACAAGGGAGCAAGC
GCACAGAGCTTCATCGAAAGAATGACAAACTTCGACAAGAACCTGCCGAACGAA
AAGGTCCTGCCGAAGCACAGCCTGCTGTACGAATACTTCACAGTCTACAACGAA
CTGACAAAGGTCAAGTACGTCACAGAAGGAATGAGAAAGCCGGCATTCCTGAGC
GGAGAACAGAAGAAGGCAATCGTCGACCTGCTGTTCAAGACAAACAGAAAGGT
CACAGTCAAGCAGCTGAAGGAAGACTACTTCAAGAAGATCGAATGCTTCGACAG
CGTCGAAATCAGCGGAGTCGAAGACAGATTCAACGCAAGCCTGGGAACATACCA
CGACCTGCTGAAGATCATCAAGGACAAGGACTTCCTGGACAACGAAGAAAACGA
AGACATCCTGGAAGACATCGTCCTGACACTGACACTGTTCGAAGACAGAGAAAT
GATCGAAGAAAGACTGAAGACATACGCACACCTGTTCGACGACAAGGTCATGAA
GCAGCTGAAGAGAAGAAGATACACAGGATGGGGAAGACTGAGCAGAAAGCTGA
TCAACGGAATCAGAGACAAGCAGAGCGGAAAGACAATCCTGGACTTCCTGAAGA
GCGACGGATTCGCAAACAGAAACTTCATGCAGCTGATCCACGACGACAGCCTGA
CATTCAAGGAAGACATCCAGAAGGCACAGGTCAGCGGACAGGGAGACAGCCTG
CACGAACACATCGCAAACCTGGCAGGAAGCCCGGCAATCAAGAAGGGAATCCTG
CAGACAGTCAAGGTCGTCGACGAACTGGTCAAGGTCATGGGAAGACACAAGCCG
GAAAACATCGTCATCGAAATGGCAAGAGAAAACCAGACAACACAGAAGGGACA
GAAGAACAGCAGAGAAAGAATGAAGAGAATCGAAGAAGGAATCAAGGAACTGG
GAAGCCAGATCCTGAAGGAACACCCGGTCGAAAACACACAGCTGCAGAACGAA
AAGCTGTACCTGTACTACCTGCAGAACGGAAGAGACATGTACGTCGACCAGGAA
CTGGACATCAACAGACTGAGCGACTACGACGTCGACCACATCGTCCCGCAGAGC
TTCCTGAAGGACGACAGCATCGACAACAAGGTCCTGACAAGAAGCGACAAGAAC
AGAGGAAAGAGCGACAACGTCCCGAGCGAAGAAGTCGTCAAGAAGATGAAGAA
CTACTGGAGACAGCTGCTGAACGCAAAGCTGATCACACAGAGAAAGTTCGACAA
CCTGACAAAGGCAGAGAGAGGAGGACTGAGCGAACTGGACAAGGCAGGATTCA
TCAAGAGACAGCTGGTCGAAACAAGACAGATCACAAAGCACGTCGCACAGATCC
TGGACAGCAGAATGAACACAAAGTACGACGAAAACGACAAGCTGATCAGAGAA
GTCAAGGTCATCACACTGAAGAGCAAGCTGGTCAGCGACTTCAGAAAGGACTTC
CAGTTCTACAAGGTCAGAGAAATCAACAACTACCACCACGCACACGACGCATAC
CTGAACGCAGTCGTCGGAACAGCACTGATCAAGAAGTACCCGAAGCTGGAAAGC
GAATTCGTCTACGGAGACTACAAGGTCTACGACGTCAGAAAGATGATCGCAAAG
AGCGAACAGGAAATCGGAAAGGCAACAGCAAAGTACTTCTTCTACAGCAACATC
ATGAACTTCTTCAAGACAGAAATCACACTGGCAAACGGAGAAATCAGAAAGAGA
CCGCTGATCGAAACAAACGGAGAAACAGGAGAAATCGTCTGGGACAAGGGAAG
AGACTTCGCAACAGTCAGAAAGGTCCTGAGCATGCCGCAGGTCAACATCGTCAA
GAAGACAGAAGTCCAGACAGGAGGATTCAGCAAGGAAAGCATCCTGCCGAAGA
GAAACAGCGACAAGCTGATCGCAAGAAAGAAGGACTGGGACCCGAAGAAGTAC
GGAGGATTCGACAGCCCGACAGTCGCATACAGCGTCCTGGTCGTCGCAAAGGTC
GAAAAGGGAAAGAGCAAGAAGCTGAAGAGCGTCAAGGAACTGCTGGGAATCAC
AATCATGGAAAGAAGCAGCTTCGAAAAGAACCCGATCGACTTCCTGGAAGCAAA
GGGATACAAGGAAGTCAAGAAGGACCTGATCATCAAGCTGCCGAAGTACAGCCT
GTTCGAACTGGAAAACGGAAGAAAGAGAATGCTGGCAAGCGCAGGAGAACTGC
AGAAGGGAAACGAACTGGCACTGCCGAGCAAGTACGTCAACTTCCTGTACCTGG
CAAGCCACTACGAAAAGCTGAAGGGAAGCCCGGAAGACAACGAACAGAAGCAG
CTGTTCGTCGAACAGCACAAGCACTACCTGGACGAAATCATCGAACAGATCAGC
GAATTCAGCAAGAGAGTCATCCTGGCAGACGCAAACCTGGACAAGGTCCTGAGC
GCATACAACAAGCACAGAGACAAGCCGATCAGAGAACAGGCAGAAAACATCAT
CCACCTGTTCACACTGACAAACCTGGGAGCACCGGCAGCATTCAAGTACTTCGAC
ACAACAATCGACAGAAAGAGATACACAAGCACAAAGGAAGTCCTGGACGCAAC
ACTGATCCACCAGAGCATCACAGGACTGTACGAAACAAGAATCGACCTGAGCCA
GCTGGGAGGAGACGGAGGAGGAAGCCCGAAGAAGAAGAGAAAGGTCTAGCTAG
CACCAGCCTCAAGAACACCCGAATGGAGTCTCTAAGCTACATAATACCAACTTA
CACTTTACAAAATGTTGTCCCCCAAAATGTAGCCATTCGTATCTGCTCCTAATAA
AAAGAAAGTTTCTTCACATTCTCTCGAG
SpCas9 mRNA #10
[SEQ ID NO: 1359]
AGGTCCCGCAGTCGGCGTCCAGCGGCTCTGCTTGTTCGTGTGTGTGTCGTTGCAG
GCCTTATTCGGATCCGCCACCATGGACAAGAAGTACAGCATCGGACTGGACATC
GGAACAAACAGCGTCGGATGGGCAGTCATCACAGACGAATACAAGGTCCCGAGC
AAGAAGTTCAAGGTCCTGGGAAACACAGACAGACACAGCATCAAGAAGAACCT
GATCGGAGCACTGCTGTTCGACAGCGGAGAAACAGCAGAAGCAACAAGACTGA
AGAGAACAGCAAGAAGAAGATACACAAGAAGAAAGAACAGAATCTGCTACCTG
CAGGAAATCTTCAGCAACGAAATGGCAAAGGTCGACGACAGCTTCTTCCACAGA
CTGGAAGAAAGCTTCCTGGTCGAAGAAGACAAGAAGCACGAAAGACACCCGAT
CTTCGGAAACATCGTCGACGAAGTCGCATACCACGAAAAGTACCCGACAATCTA
CCACCTGAGAAAGAAGCTGGTCGACAGCACAGACAAGGCAGACCTGAGACTGAT
CTACCTGGCACTGGCACACATGATCAAGTTCAGAGGACACTTCCTGATCGAAGG
AGACCTGAACCCGGACAACAGCGACGTCGACAAGCTGTTCATCCAGCTGGTCCA
GACATACAACCAGCTGTTCGAAGAAAACCCGATCAACGCAAGCGGAGTCGACGC
AAAGGCAATCCTGAGCGCAAGACTGAGCAAGAGCAGAAGACTGGAAAACCTGA
TCGCACAGCTGCCGGGAGAAAAGAAGAACGGACTGTTCGGAAACCTGATCGCAC
TGAGCCTGGGACTGACACCGAACTTCAAGAGCAACTTCGACCTGGCAGAAGACG
CAAAGCTGCAGCTGAGCAAGGACACATACGACGACGACCTGGACAACCTGCTGG
CACAGATCGGAGACCAGTACGCAGACCTGTTCCTGGCAGCAAAGAACCTGAGCG
ACGCAATCCTGCTGAGCGACATCCTGAGAGTCAACACAGAAATCACAAAGGCAC
CGCTGAGCGCAAGCATGATCAAGAGATACGACGAACACCACCAGGACCTGACAC
TGCTGAAGGCACTGGTCAGACAGCAGCTGCCGGAAAAGTACAAGGAAATCTTCT
TCGACCAGAGCAAGAACGGATACGCAGGATACATCGACGGAGGAGCAAGCCAG
GAAGAATTCTACAAGTTCATCAAGCCGATCCTGGAAAAGATGGACGGAACAGAA
GAACTGCTGGTCAAGCTGAACAGAGAAGACCTGCTGAGAAAGCAGAGAACATTC
GACAACGGAAGCATCCCGCACCAGATCCACCTGGGAGAACTGCACGCAATCCTG
AGAAGACAGGAAGACTTCTACCCGTTCCTGAAGGACAACAGAGAAAAGATCGA
AAAGATCCTGACATTCAGAATCCCGTACTACGTCGGACCGCTGGCAAGAGGAAA
CAGCAGATTCGCATGGATGACAAGAAAGAGCGAAGAAACAATCACACCGTGGA
ACTTCGAAGAAGTCGTCGACAAGGGAGCAAGCGCACAGAGCTTCATCGAAAGAA
TGACAAACTTCGACAAGAACCTGCCGAACGAAAAGGTCCTGCCGAAGCACAGCC
TGCTGTACGAATACTTCACAGTCTACAACGAACTGACAAAGGTCAAGTACGTCA
CAGAAGGAATGAGAAAGCCGGCATTCCTGAGCGGAGAACAGAAGAAGGCAATC
GTCGACCTGCTGTTCAAGACAAACAGAAAGGTCACAGTCAAGCAGCTGAAGGAA
GACTACTTCAAGAAGATCGAATGCTTCGACAGCGTCGAAATCAGCGGAGTCGAA
GACAGATTCAACGCAAGCCTGGGAACATACCACGACCTGCTGAAGATCATCAAG
GACAAGGACTTCCTGGACAACGAAGAAAACGAAGACATCCTGGAAGACATCGTC
CTGACACTGACACTGTTCGAAGACAGAGAAATGATCGAAGAAAGACTGAAGACA
TACGCACACCTGTTCGACGACAAGGTCATGAAGCAGCTGAAGAGAAGAAGATAC
ACAGGATGGGGAAGACTGAGCAGAAAGCTGATCAACGGAATCAGAGACAAGCA
GAGCGGAAAGACAATCCTGGACTTCCTGAAGAGCGACGGATTCGCAAACAGAAA
CTTCATGCAGCTGATCCACGACGACAGCCTGACATTCAAGGAAGACATCCAGAA
GGCACAGGTCAGCGGACAGGGAGACAGCCTGCACGAACACATCGCAAACCTGG
CAGGAAGCCCGGCAATCAAGAAGGGAATCCTGCAGACAGTCAAGGTCGTCGACG
AACTGGTCAAGGTCATGGGAAGACACAAGCCGGAAAACATCGTCATCGAAATGG
CAAGAGAAAACCAGACAACACAGAAGGGACAGAAGAACAGCAGAGAAAGAAT
GAAGAGAATCGAAGAAGGAATCAAGGAACTGGGAAGCCAGATCCTGAAGGAAC
ACCCGGTCGAAAACACACAGCTGCAGAACGAAAAGCTGTACCTGTACTACCTGC
AGAACGGAAGAGACATGTACGTCGACCAGGAACTGGACATCAACAGACTGAGC
GACTACGACGTCGACCACATCGTCCCGCAGAGCTTCCTGAAGGACGACAGCATC
GACAACAAGGTCCTGACAAGAAGCGACAAGAACAGAGGAAAGAGCGACAACGT
CCCGAGCGAAGAAGTCGTCAAGAAGATGAAGAACTACTGGAGACAGCTGCTGA
ACGCAAAGCTGATCACACAGAGAAAGTTCGACAACCTGACAAAGGCAGAGAGA
GGAGGACTGAGCGAACTGGACAAGGCAGGATTCATCAAGAGACAGCTGGTCGA
AACAAGACAGATCACAAAGCACGTCGCACAGATCCTGGACAGCAGAATGAACA
CAAAGTACGACGAAAACGACAAGCTGATCAGAGAAGTCAAGGTCATCACACTGA
AGAGCAAGCTGGTCAGCGACTTCAGAAAGGACTTCCAGTTCTACAAGGTCAGAG
AAATCAACAACTACCACCACGCACACGACGCATACCTGAACGCAGTCGTCGGAA
CAGCACTGATCAAGAAGTACCCGAAGCTGGAAAGCGAATTCGTCTACGGAGACT
ACAAGGTCTACGACGTCAGAAAGATGATCGCAAAGAGCGAACAGGAAATCGGA
AAGGCAACAGCAAAGTACTTCTTCTACAGCAACATCATGAACTTCTTCAAGACA
GAAATCACACTGGCAAACGGAGAAATCAGAAAGAGACCGCTGATCGAAACAAA
CGGAGAAACAGGAGAAATCGTCTGGGACAAGGGAAGAGACTTCGCAACAGTCA
GAAAGGTCCTGAGCATGCCGCAGGTCAACATCGTCAAGAAGACAGAAGTCCAGA
CAGGAGGATTCAGCAAGGAAAGCATCCTGCCGAAGAGAAACAGCGACAAGCTG
ATCGCAAGAAAGAAGGACTGGGACCCGAAGAAGTACGGAGGATTCGACAGCCC
GACAGTCGCATACAGCGTCCTGGTCGTCGCAAAGGTCGAAAAGGGAAAGAGCAA
GAAGCTGAAGAGCGTCAAGGAACTGCTGGGAATCACAATCATGGAAAGAAGCA
GCTTCGAAAAGAACCCGATCGACTTCCTGGAAGCAAAGGGATACAAGGAAGTCA
AGAAGGACCTGATCATCAAGCTGCCGAAGTACAGCCTGTTCGAACTGGAAAACG
GAAGAAAGAGAATGCTGGCAAGCGCAGGAGAACTGCAGAAGGGAAACGAACTG
GCACTGCCGAGCAAGTACGTCAACTTCCTGTACCTGGCAAGCCACTACGAAAAG
CTGAAGGGAAGCCCGGAAGACAACGAACAGAAGCAGCTGTTCGTCGAACAGCA
CAAGCACTACCTGGACGAAATCATCGAACAGATCAGCGAATTCAGCAAGAGAGT
CATCCTGGCAGACGCAAACCTGGACAAGGTCCTGAGCGCATACAACAAGCACAG
AGACAAGCCGATCAGAGAACAGGCAGAAAACATCATCCACCTGTTCACACTGAC
AAACCTGGGAGCACCGGCAGCATTCAAGTACTTCGACACAACAATCGACAGAAA
GAGATACACAAGCACAAAGGAAGTCCTGGACGCAACACTGATCCACCAGAGCAT
CACAGGACTGTACGAAACAAGAATCGACCTGAGCCAGCTGGGAGGAGACGGAG
GAGGAAGCCCGAAGAAGAAGAGAAAGGTCTAGCTAGCCATCACATTTAAAAGC
ATCTCAGCCTACCATGAGAATAAGAGAAAGAAAATGAAGATCAATAGCTTATTC
ATCTCTTTTTCTTTTTCGTTGGTGTAAAGCCAACACCCTGTCTAAAAAACATAAAT
TTCTTTAATCATTTTGCCTCTTTTCTCTGTGCTTCAATTAATAAAAAATGGAAAGA
ACCTCGAG
SpCas9 mRNA #11
[SEQ ID NO: 1480]
AGGAAAUAAGAGAGAAAAGAAGAGUAAGAAGAAAUAUAAGAGCCACCAUGGA
CAAGAAGUACAGCAUCGGCCUGGACAUCGGCACCAACUCUGUGGGCUGGGCCG
UGAUCACCGACGAGUACAAGGUGCCCAGCAAGAAAUUCAAGGUGCUGGGCAAC
ACCGACCGGCACAGCAUCAAGAAGAACCUGAUCGGAGCCCUGCUGUUCGACAG
CGGCGAAACAGCCGAGGCCACCCGGCUGAAGAGAACCGCCAGAAGAAGAUACA
CCAGACGGAAGAACCGGAUCUGCUAUCUGCAAGAGAUCUUCAGCAACGAGAUG
GCCAAGGUGGACGACAGCUUCUUCCACAGACUGGAAGAGUCCUUCCUGGUGGA
AGAGGAUAAGAAGCACGAGCGGCACCCCAUCUUCGGCAACAUCGUGGACGAGG
UGGCCUACCACGAGAAGUACCCCACCAUCUACCACCUGAGAAAGAAACUGGUG
GACAGCACCGACAAGGCCGACCUGCGGCUGAUCUAUCUGGCCCUGGCCCACAU
GAUCAAGUUCCGGGGCCACUUCCUGAUCGAGGGCGACCUGAACCCCGACAACA
GCGACGUGGACAAGCUGUUCAUCCAGCUGGUGCAGACCUACAACCAGCUGUUC
GAGGAAAACCCCAUCAACGCCAGCGGCGUGGACGCCAAGGCCAUCCUGUCUGC
CAGACUGAGCAAGAGCAGACGGCUGGAAAAUCUGAUCGCCCAGCUGCCCGGCG
AGAAGAAGAAUGGCCUGUUCGGAAACCUGAUUGCCCUGAGCCUGGGCCUGACC
CCCAACUUCAAGAGCAACUUCGACCUGGCCGAGGAUGCCAAACUGCAGCUGAG
CAAGGACACCUACGACGACGACCUGGACAACCUGCUGGCCCAGAUCGGCGACC
AGUACGCCGACCUGUUUCUGGCCGCCAAGAACCUGUCCGACGCCAUCCUGCUG
AGCGACAUCCUGAGAGUGAACACCGAGAUCACCAAGGCCCCCCUGAGCGCCUC
UAUGAUCAAGAGAUACGACGAGCACCACCAGGACCUGACCCUGCUGAAAGCUC
UCGUGCGGCAGCAGCUGCCUGAGAAGUACAAAGAGAUUUUCUUCGACCAGAGC
AAGAACGGCUACGCCGGCUACAUUGACGGCGGAGCCAGCCAGGAAGAGUUCUA
CAAGUUCAUCAAGCCCAUCCUGGAAAAGAUGGACGGCACCGAGGAACUGCUCG
UGAAGCUGAACAGAGAGGACCUGCUGCGGAAGCAGCGGACCUUCGACAACGGC
AGCAUCCCCCACCAGAUCCACCUGGGAGAGCUGCACGCCAUUCUGCGGCGGCA
GGAAGAUUUUUACCCAUUCCUGAAGGACAACCGGGAAAAGAUCGAGAAGAUC
CUGACCUUCCGCAUCCCCUACUACGUGGGCCCUCUGGCCAGGGGAAACAGCAG
AUUCGCCUGGAUGACCAGAAAGAGCGAGGAAACCAUCACCCCCUGGAACUUCG
AGGAAGUGGUGGACAAGGGCGCUUCCGCCCAGAGCUUCAUCGAGCGGAUGACC
AACUUCGAUAAGAACCUGCCCAACGAGAAGGUGCUGCCCAAGCACAGCCUGCU
GUACGAGUACUUCACCGUGUAUAACGAGCUGACCAAAGUGAAAUACGUGACC
GAGGGAAUGAGAAAGCCCGCCUUCCUGAGCGGCGAGCAGAAAAAGGCCAUCGU
GGACCUGCUGUUCAAGACCAACCGGAAAGUGACCGUGAAGCAGCUGAAAGAG
GACUACUUCAAGAAAAUCGAGUGCUUCGACUCCGUGGAAAUCUCCGGCGUGGA
AGAUCGGUUCAACGCCUCCCUGGGCACAUACCACGAUCUGCUGAAAAUUAUCA
AGGACAAGGACUUCCUGGACAAUGAGGAAAACGAGGACAUUCUGGAAGAUAU
CGUGCUGACCCUGACACUGUUUGAGGACAGAGAGAUGAUCGAGGAACGGCUG
AAAACCUAUGCCCACCUGUUCGACGACAAAGUGAUGAAGCAGCUGAAGCGGCG
GAGAUACACCGGCUGGGGCAGGCUGAGCCGGAAGCUGAUCAACGGCAUCCGGG
ACAAGCAGUCCGGCAAGACAAUCCUGGAUUUCCUGAAGUCCGACGGCUUCGCC
AACAGAAACUUCAUGCAGCUGAUCCACGACGACAGCCUGACCUUUAAAGAGGA
CAUCCAGAAAGCCCAGGUGUCCGGCCAGGGCGAUAGCCUGCACGAGCACAUUG
CCAAUCUGGCCGGCAGCCCCGCCAUUAAGAAGGGCAUCCUGCAGACAGUGAAG
GUGGUGGACGAGCUCGUGAAAGUGAUGGGCCGGCACAAGCCCGAGAACAUCG
UGAUCGAAAUGGCCAGAGAGAACCAGACCACCCAGAAGGGACAGAAGAACAGC
CGCGAGAGAAUGAAGCGGAUCGAAGAGGGCAUCAAAGAGCUGGGCAGCCAGA
UCCUGAAAGAACACCCCGUGGAAAACACCCAGCUGCAGAACGAGAAGCUGUAC
CUGUACUACCUGCAGAAUGGGCGGGAUAUGUACGUGGACCAGGAACUGGACA
UCAACCGGCUGUCCGACUACGAUGUGGACCAUAUCGUGCCUCAGAGCUUUCUG
AAGGACGACUCCAUCGACAACAAGGUGCUGACCAGAAGCGACAAGAACCGGGG
CAAGAGCGACAACGUGCCCUCCGAAGAGGUCGUGAAGAAGAUGAAGAACUAC
UGGCGGCAGCUGCUGAACGCCAAGCUGAUUACCCAGAGAAAGUUCGACAAUCU
GACCAAGGCCGAGAGAGGCGGCCUGAGCGAACUGGAUAAGGCCGGCUUCAUCA
AGAGACAGCUGGUGGAAACCCGGCAGAUCACAAAGCACGUGGCACAGAUCCUG
GACUCCCGGAUGAACACUAAGUACGACGAGAAUGACAAGCUGAUCCGGGAAG
UGAAAGUGAUCACCCUGAAGUCCAAGCUGGUGUCCGAUUUCCGGAAGGAUUU
CCAGUUUUACAAAGUGCGCGAGAUCAACAACUACCACCACGCCCACGACGCCU
ACCUGAACGCCGUCGUGGGAACCGCCCUGAUCAAAAAGUACCCUAAGCUGGAA
AGCGAGUUCGUGUACGGCGACUACAAGGUGUACGACGUGCGGAAGAUGAUCG
CCAAGAGCGAGCAGGAAAUCGGCAAGGCUACCGCCAAGUACUUCUUCUACAGC
AACAUCAUGAACUUUUUCAAGACCGAGAUUACCCUGGCCAACGGCGAGAUCCG
GAAGCGGCCUCUGAUCGAGACAAACGGCGAAACCGGGGAGAUCGUGUGGGAU
AAGGGCCGGGAUUUUGCCACCGUGCGGAAAGUGCUGAGCAUGCCCCAAGUGAA
UAUCGUGAAAAAGACCGAGGUGCAGACAGGCGGCUUCAGCAAAGAGUCUAUC
CUGCCCAAGAGGAACAGCGAUAAGCUGAUCGCCAGAAAGAAGGACUGGGACCC
UAAGAAGUACGGCGGCUUCGACAGCCCCACCGUGGCCUAUUCUGUGCUGGUGG
UGGCCAAAGUGGAAAAGGGCAAGUCCAAGAAACUGAAGAGUGUGAAAGAGCU
GCUGGGGAUCACCAUCAUGGAAAGAAGCAGCUUCGAGAAGAAUCCCAUCGACU
UUCUGGAAGCCAAGGGCUACAAAGAAGUGAAAAAGGACCUGAUCAUCAAGCU
GCCUAAGUACUCCCUGUUCGAGCUGGAAAACGGCCGGAAGAGAAUGCUGGCCU
CUGCCGGCGAACUGCAGAAGGGAAACGAACUGGCCCUGCCCUCCAAAUAUGUG
AACUUCCUGUACCUGGCCAGCCACUAUGAGAAGCUGAAGGGCUCCCCCGAGGA
UAAUGAGCAGAAACAGCUGUUUGUGGAACAGCACAAGCACUACCUGGACGAG
AUCAUCGAGCAGAUCAGCGAGUUCUCCAAGAGAGUGAUCCUGGCCGACGCUAA
UCUGGACAAAGUGCUGUCCGCCUACAACAAGCACCGGGAUAAGCCCAUCAGAG
AGCAGGCCGAGAAUAUCAUCCACCUGUUUACCCUGACCAAUCUGGGAGCCCCU
GCCGCCUUCAAGUACUUUGACACCACCAUCGACCGGAAGAGGUACACCAGCAC
CAAAGAGGUGCUGGACGCCACCCUGAUCCACCAGAGCAUCACCGGCCUGUACG
AGACACGGAUCGACCUGUCUCAGCUGGGAGGUGACGGCGGAUCCCCUGCUGCU
AAACGUGUUAAGCUUGAUGGGGGUAGCCCGGCAGCCAAGAGAGUCAAACUCG
ACUAGAUGGUUUAUAUUGCGGCCGCUUAAUUAAGCUGCCUUCUGCGGGGCUU
GCCUUCUGGCCAUGCCCUUCUUCUCUCCCUUGCACCUGUACCUCUUGGUCUUU
GAAUAAAGCCUGAGUAGGAAGUCUAGAAAAAAAAAAAAAAAAAAAAAAAAAA
AAAAAAAAAAAAAAAAAAAAAAAAAAAAAAAAAAAAAAAAAAAAAAAAAAAA
AA

5.4.1 Cas12a mRNAs

AsCas12a mRNA #1
(5′ modification is CleanCap ® AG; 3′Tail Modification is a 79A poly A sequence)
AGGAAAUAAGAGAGAAAAGAAGAGUAAGAAGAAAUAUAAGAGCCACCAUGAC
ACAGUUCGAGGGCUUUACCAACCUGUAUCAGGUGAGCAAGACACUGCGGUUU
GAGCUGAUCCCACAGGGCAAGACCCUGAAGCACAUCCAGGAGCAGGGCUUCAU
CGAGGAGGACAAGGCCCGCAAUGAUCACUACAAGGAGCUGAAGCCCAUCAUCG
AUCGGAUCUACAAGACCUAUGCCGACCAGUGCCUGCAGCUGGUGCAGCUGGAU
UGGGAGAACCUGAGCGCCGCCAUCGACUCCUAUAGAAAGGAGAAAACCGAGGA
GACAAGGAACGCCCUGAUCGAGGAGCAGGCCACAUAUCGCAAUGCCAUCCACG
ACUACUUCAUCGGCCGGACAGACAACCUGACCGAUGCCAUCAAUAAGAGACAC
GCCGAGAUCUACAAGGGCCUGUUCAAGGCCGAGCUGUUUAAUGGCAAGGUGC
UGAAGCAGCUGGGCACCGUGACCACAACCGAGCACGAGAACGCCCUGCUGCGG
AGCUUCGACAAGUUUACAACCUACUUCUCCGGCUUUUAUGAGAACAGGAAGA
ACGUGUUCAGCGCCGAGGAUAUCAGCACAGCCAUCCCACACCGCAUCGUGCAG
GACAACUUCCCCAAGUUUAAGGAGAAUUGUCACAUCUUCACACGCCUGAUCAC
CGCCGUGCCCAGCCUGCGGGAGCACUUUGAGAACGUGAAGAAGGCCAUCGGCA
UCUUCGUGAGCACCUCCAUCGAGGAGGUGUUUUCCUUCCCUUUUUAUAACCAG
CUGCUGACACAGACCCAGAUCGACCUGUAUAACCAGCUGCUGGGAGGAAUCUC
UCGGGAGGCAGGCACCGAGAAGAUCAAGGGCCUGAACGAGGUGCUGAAUCUG
GCCAUCCAGAAGAAUGAUGAGACAGCCCACAUCAUCGCCUCCCUGCCACACAG
AUUCAUCCCCCUGUUUAAGCAGAUCCUGUCCGAUAGGAACACCCUGUCUUUCA
UCCUGGAGGAGUUUAAGAGCGACGAGGAAGUGAUCCAGUCCUUCUGCAAGUA
CAAGACACUGCUGAGAAACGAGAACGUGCUGGAGACAGCCGAGGCCCUGUUUA
ACGAGCUGAACAGCAUCGACCUGACACACAUCUUCAUCAGCCACAAGAAGCUG
GAGACAAUCAGCAGCGCCCUGUGCGACCACUGGGAUACACUGAGGAAUGCCCU
GUAUGAGCGGAGAAUCUCCGAGCUGACAGGCAAGAUCACCAAGUCUGCCAAGG
AGAAGGUGCAGCGCAGCCUGAAGCACGAGGAUAUCAACCUGCAGGAGAUCAUC
UCUGCCGCAGGCAAGGAGCUGAGCGAGGCCUUCAAGCAGAAAACCAGCGAGAU
CCUGUCCCACGCACACGCCGCCCUGGAUCAGCCACUGCCUACAACCCUGAAGA
AGCAGGAGGAGAAGGAGAUCCUGAAGUCUCAGCUGGACAGCCUGCUGGGCCU
GUACCACCUGCUGGACUGGUUUGCCGUGGAUGAGUCCAACGAGGUGGACCCCG
AGUUCUCUGCCCGGCUGACCGGCAUCAAGCUGGAGAUGGAGCCUUCUCUGAGC
UUCUACAACAAGGCCAGAAAUUAUGCCACCAAGAAGCCCUACUCCGUGGAGAA
GUUCAAGCUGAACUUUCAGCGGCCUACACUGGCCUCUGGCUGGGACGUGAAUA
AGGAGAAGAACAAUGGCGCCAUCCUGUUUGUGAAGAACGGCCUGUACUAUCU
GGGCAUCAUGCCAAAGCAGAAGGGCAGGUAUAAGGCCCUGAGCUUCGAGCCCA
CAGAGAAAACCAGCGAGGGCUUUGAUAAGAUGUACUAUGACUACUUCCCUGA
UGCCGCCAAGAUGAUCCCAAAGUGCAGCACCCAGCUGAAGGCCGUGACAGCCC
ACUUUCAGACCCACACAACCCCCAUCCUGCUGUCCAACAAUUUCAUCGAGCCU
CUGGAGAUCACAAAGGAGAUCUACGACCUGAACAAUCCUGAGAAGGAGCCAA
AGAAGUUUCAGACAGCCUACGCCAAGAAAACCGGCGACCAGAAGGGCUACAGA
GAGGCCCUGUGCAAGUGGAUCGACUUCACAAGGGAUUUUCUGUCCAAGUAUA
CCAAGACAACCUCUAUCGAUCUGUCUAGCCUGCGGCCAUCCUCUCAGUAUAAG
GACCUGGGCGAGUACUAUGCCGAGCUGAAUCCCCUGCUGUACCACAUCAGCUU
CCAGAGAAUCGCCGAGAAGGAGAUCAUGGAUGCCGUGGAGACAGGCAAGCUG
UACCUGUUCCAGAUCUAUAACAAGGACUUUGCCAAGGGCCACCACGGCAAGCC
UAAUCUGCACACACUGUAUUGGACCGGCCUGUUUUCUCCAGAGAACCUGGCCA
AGACAAGCAUCAAGCUGAAUGGCCAGGCCGAGCUGUUCUACCGCCCUAAGUCC
AGGAUGAAGAGGAUGGCAGCCCGGCUGGGAGAGAAGAUGCUGAACAAGAAGC
UGAAGGAUCAGAAAACCCCAAUCCCCGACACCCUGUACCAGGAGCUGUACGAC
UAUGUGAAUCACAGACUGUCCCACGACCUGUCUGAUGAGGCCAGGGCCCUGCU
GCCCAACGUGAUCACCAAGGAGGUGUCUCACGAGAUCAUCAAGGAUAGGCGCU
UUACCAGCGACAAGUUCUUGUUCCACGUGCCUAUCACACUGAACUAUCAGGCC
GCCAAUUCCCCAUCUAAGUUCAACCAGAGGGUGAAUGCCUACCUGAAGGAGCA
CCCCGAGACACCUAUCAUCGGCAUCGAUCGGGGCGAGAGAAACCUGAUCUAUA
UCACAGUGAUCGACUCCACCGGCAAGAUCCUGGAGCAGCGGAGCCUGAACACC
AUCCAGCAGUUUGAUUACCAGAAGAAGCUGGACAACAGGGAGAAGGAGAGGG
UGGCAGCAAGGCAGGCCUGGUCUGUGGUGGGCACAAUCAAGGAUCUGAAGCA
GGGCUAUCUGAGCCAGGUCAUCCACGAGAUCGUGGACCUGAUGAUCCACUACC
AGGCCGUGGUGGUGCUGGAGAACCUGAAUUUCGGCUUUAAGAGCAAGAGGAC
CGGCAUCGCCGAGAAGGCCGUGUACCAGCAGUUCGAGAAGAUGCUGAUCGAUA
AGCUGAAUUGCCUGGUGCUGAAGGACUAUCCAGCAGAGAAAGUGGGAGGCGU
GCUGAACCCAUACCAGCUGACAGACCAGUUCACCUCCUUUGCCAAGAUGGGCA
CCCAGUCUGGCUUCCUGUUUUACGUGCCUGCCCCAUAUACAUCUAAGAUCGAU
CCCCUGACCGGCUUCGUGGACCCCUUCGUGUGGAAAACCAUCAAGAAUCACGA
GAGCCGCAAGCACUUCCUGGAGGGCUUCGACUUUCUGCACUACGACGUGAAAA
CCGGCGACUUCAUCCUGCACUUUAAGAUGAACAGAAAUCUGUCCUUCCAGAGG
GGCCUGCCCGGCUUUAUGCCUGCAUGGGAUAUCGUGUUCGAGAAGAACGAGAC
ACAGUUUGACGCCAAGGGCACCCCUUUCAUCGCCGGCAAGAGAAUCGUGCCAG
UGAUCGAGAAUCACAGAUUCACCGGCAGAUACCGGGACCUGUAUCCUGCCAAC
GAGCUGAUCGCCCUGCUGGAGGAGAAGGGCAUCGUGUUCAGGGAUGGCUCCA
ACAUCCUGCCAAAGCUGCUGGAGAAUGACGAUUCUCACGCCAUCGACACCAUG
GUGGCCCUGAUCCGCAGCGUGCUGCAGAUGCGGAACUCCAAUGCCGCCACAGG
CGAGGACUAUAUCAACAGCCCCGUGCGCGAUCUGAAUGGCGUGUGCUUCGACU
CCCGGUUUCAGAACCCAGAGUGGCCCAUGGACGCCGAUGCCAAUGGCGCCUAC
CACAUCGCCCUGAAGGGCCAGCUGCUGCUGAAUCACCUGAAGGAGAGCAAGGA
UCUGAAGCUGCAGAACGGCAUCUCCAAUCAGGACUGGCUGGCCUACAUCCAGG
AGCUGCGCAACGGUCGUAGCAGUGAUGAUGAAGCAACCGCAGAUAGCCAGCAU
GCAGCACCGCCCAAGAAGAAGAGGAAAGUCUAAUAGUGAAUGGUUUAUAUUG
CGGCCGCUUAAUUAAGCUGCCUUCUGCGGGGCUUGCCUUCUGGCCAUGCCCUU
CUUCUCUCCCUUGCACCUGUACCUCUUGGUCUUUGAAUAAAGCCUGAGUAGGA
AGUCUAGAAAAAAAAAAAAAAAAAAAAAAAAAAAAAAAAAAAAAAAAAAAAA
AAAAAAAAAAAAAAAAAAAAAAAAAAAAAAAAAA [SEQ ID NO: 895]
AsCas12a mRNA #2
(5′ Modification = CleanCap ® AG 3′Ome; Uridine Modifications = N1-Methyl-Pseudo UTP;
3′ Tail Modification = 79A tail)
AGGAAAUAAGAGAGAAAAGAAGAGUAAGAAGAAAUAUAAGAGCCACCAUGAC
ACAGUUCGAGGGCUUUACCAACCUGUAUCAGGUGAGCAAGACACUGCGGUUU
GAGCUGAUCCCACAGGGCAAGACCCUGAAGCACAUCCAGGAGCAGGGCUUCAU
CGAGGAGGACAAGGCCCGCAAUGAUCACUACAAGGAGCUGAAGCCCAUCAUCG
AUCGGAUCUACAAGACCUAUGCCGACCAGUGCCUGCAGCUGGUGCAGCUGGAU
UGGGAGAACCUGAGCGCCGCCAUCGACUCCUAUAGAAAGGAGAAAACCGAGGA
GACAAGGAACGCCCUGAUCGAGGAGCAGGCCACAUAUCGCAAUGCCAUCCACG
ACUACUUCAUCGGCCGGACAGACAACCUGACCGAUGCCAUCAAUAAGAGACAC
GCCGAGAUCUACAAGGGCCUGUUCAAGGCCGAGCUGUUUAAUGGCAAGGUGC
UGAAGCAGCUGGGCACCGUGACCACAACCGAGCACGAGAACGCCCUGCUGCGG
AGCUUCGACAAGUUUACAACCUACUUCUCCGGCUUUUAUGAGAACAGGAAGA
ACGUGUUCAGCGCCGAGGAUAUCAGCACAGCCAUCCCACACCGCAUCGUGCAG
GACAACUUCCCCAAGUUUAAGGAGAAUUGUCACAUCUUCACACGCCUGAUCAC
CGCCGUGCCCAGCCUGCGGGAGCACUUUGAGAACGUGAAGAAGGCCAUCGGCA
UCUUCGUGAGCACCUCCAUCGAGGAGGUGUUUUCCUUCCCUUUUUAUAACCAG
CUGCUGACACAGACCCAGAUCGACCUGUAUAACCAGCUGCUGGGAGGAAUCUC
UCGGGAGGCAGGCACCGAGAAGAUCAAGGGCCUGAACGAGGUGCUGAAUCUG
GCCAUCCAGAAGAAUGAUGAGACAGCCCACAUCAUCGCCUCCCUGCCACACAG
AUUCAUCCCCCUGUUUAAGCAGAUCCUGUCCGAUAGGAACACCCUGUCUUUCA
UCCUGGAGGAGUUUAAGAGCGACGAGGAAGUGAUCCAGUCCUUCUGCAAGUA
CAAGACACUGCUGAGAAACGAGAACGUGCUGGAGACAGCCGAGGCCCUGUUUA
ACGAGCUGAACAGCAUCGACCUGACACACAUCUUCAUCAGCCACAAGAAGCUG
GAGACAAUCAGCAGCGCCCUGUGCGACCACUGGGAUACACUGAGGAAUGCCCU
GUAUGAGCGGAGAAUCUCCGAGCUGACAGGCAAGAUCACCAAGUCUGCCAAGG
AGAAGGUGCAGCGCAGCCUGAAGCACGAGGAUAUCAACCUGCAGGAGAUCAUC
UCUGCCGCAGGCAAGGAGCUGAGCGAGGCCUUCAAGCAGAAAACCAGCGAGAU
CCUGUCCCACGCACACGCCGCCCUGGAUCAGCCACUGCCUACAACCCUGAAGA
AGCAGGAGGAGAAGGAGAUCCUGAAGUCUCAGCUGGACAGCCUGCUGGGCCU
GUACCACCUGCUGGACUGGUUUGCCGUGGAUGAGUCCAACGAGGUGGACCCCG
AGUUCUCUGCCCGGCUGACCGGCAUCAAGCUGGAGAUGGAGCCUUCUCUGAGC
UUCUACAACAAGGCCAGAAAUUAUGCCACCAAGAAGCCCUACUCCGUGGAGAA
GUUCAAGCUGAACUUUCAGCGGCCUACACUGGCCUCUGGCUGGGACGUGAAUA
AGGAGAAGAACAAUGGCGCCAUCCUGUUUGUGAAGAACGGCCUGUACUAUCU
GGGCAUCAUGCCAAAGCAGAAGGGCAGGUAUAAGGCCCUGAGCUUCGAGCCCA
CAGAGAAAACCAGCGAGGGCUUUGAUAAGAUGUACUAUGACUACUUCCCUGA
UGCCGCCAAGAUGAUCCCAAAGUGCAGCACCCAGCUGAAGGCCGUGACAGCCC
ACUUUCAGACCCACACAACCCCCAUCCUGCUGUCCAACAAUUUCAUCGAGCCU
CUGGAGAUCACAAAGGAGAUCUACGACCUGAACAAUCCUGAGAAGGAGCCAA
AGAAGUUUCAGACAGCCUACGCCAAGAAAACCGGCGACCAGAAGGGCUACAGA
GAGGCCCUGUGCAAGUGGAUCGACUUCACAAGGGAUUUUCUGUCCAAGUAUA
CCAAGACAACCUCUAUCGAUCUGUCUAGCCUGCGGCCAUCCUCUCAGUAUAAG
GACCUGGGCGAGUACUAUGCCGAGCUGAAUCCCCUGCUGUACCACAUCAGCUU
CCAGAGAAUCGCCGAGAAGGAGAUCAUGGAUGCCGUGGAGACAGGCAAGCUG
UACCUGUUCCAGAUCUAUAACAAGGACUUUGCCAAGGGCCACCACGGCAAGCC
UAAUCUGCACACACUGUAUUGGACCGGCCUGUUUUCUCCAGAGAACCUGGCCA
AGACAAGCAUCAAGCUGAAUGGCCAGGCCGAGCUGUUCUACCGCCCUAAGUCC
AGGAUGAAGAGGAUGGCAGCCCGGCUGGGAGAGAAGAUGCUGAACAAGAAGC
UGAAGGAUCAGAAAACCCCAAUCCCCGACACCCUGUACCAGGAGCUGUACGAC
UAUGUGAAUCACAGACUGUCCCACGACCUGUCUGAUGAGGCCAGGGCCCUGCU
GCCCAACGUGAUCACCAAGGAGGUGUCUCACGAGAUCAUCAAGGAUAGGCGCU
UUACCAGCGACAAGUUCUUGUUCCACGUGCCUAUCACACUGAACUAUCAGGCC
GCCAAUUCCCCAUCUAAGUUCAACCAGAGGGUGAAUGCCUACCUGAAGGAGCA
CCCCGAGACACCUAUCAUCGGCAUCGAUCGGGGCGAGAGAAACCUGAUCUAUA
UCACAGUGAUCGACUCCACCGGCAAGAUCCUGGAGCAGCGGAGCCUGAACACC
AUCCAGCAGUUUGAUUACCAGAAGAAGCUGGACAACAGGGAGAAGGAGAGGG
UGGCAGCAAGGCAGGCCUGGUCUGUGGUGGGCACAAUCAAGGAUCUGAAGCA
GGGCUAUCUGAGCCAGGUCAUCCACGAGAUCGUGGACCUGAUGAUCCACUACC
AGGCCGUGGUGGUGCUGGAGAACCUGAAUUUCGGCUUUAAGAGCAAGAGGAC
CGGCAUCGCCGAGAAGGCCGUGUACCAGCAGUUCGAGAAGAUGCUGAUCGAUA
AGCUGAAUUGCCUGGUGCUGAAGGACUAUCCAGCAGAGAAAGUGGGAGGCGU
GCUGAACCCAUACCAGCUGACAGACCAGUUCACCUCCUUUGCCAAGAUGGGCA
CCCAGUCUGGCUUCCUGUUUUACGUGCCUGCCCCAUAUACAUCUAAGAUCGAU
CCCCUGACCGGCUUCGUGGACCCCUUCGUGUGGAAAACCAUCAAGAAUCACGA
GAGCCGCAAGCACUUCCUGGAGGGCUUCGACUUUCUGCACUACGACGUGAAAA
CCGGCGACUUCAUCCUGCACUUUAAGAUGAACAGAAAUCUGUCCUUCCAGAGG
GGCCUGCCCGGCUUUAUGCCUGCAUGGGAUAUCGUGUUCGAGAAGAACGAGAC
ACAGUUUGACGCCAAGGGCACCCCUUUCAUCGCCGGCAAGAGAAUCGUGCCAG
UGAUCGAGAAUCACAGAUUCACCGGCAGAUACCGGGACCUGUAUCCUGCCAAC
GAGCUGAUCGCCCUGCUGGAGGAGAAGGGCAUCGUGUUCAGGGAUGGCUCCA
ACAUCCUGCCAAAGCUGCUGGAGAAUGACGAUUCUCACGCCAUCGACACCAUG
GUGGCCCUGAUCCGCAGCGUGCUGCAGAUGCGGAACUCCAAUGCCGCCACAGG
CGAGGACUAUAUCAACAGCCCCGUGCGCGAUCUGAAUGGCGUGUGCUUCGACU
CCCGGUUUCAGAACCCAGAGUGGCCCAUGGACGCCGAUGCCAAUGGCGCCUAC
CACAUCGCCCUGAAGGGCCAGCUGCUGCUGAAUCACCUGAAGGAGAGCAAGGA
UCUGAAGCUGCAGAACGGCAUCUCCAAUCAGGACUGGCUGGCCUACAUCCAGG
AGCUGCGCAACGGUCGUAGCAGUGAUGAUGAAGCAACCGCAGAUAGCCAGCAU
GCAGCACCGCCCAAGAAGAAGAGGAAAGUCUAAUAGUGAAUGGUUUAUAUUG
CGGCCGCUUAAUUAAGCUGCCUUCUGCGGGGCUUGCCUUCUGGCCAUGCCCUU
CUUCUCUCCCUUGCACCUGUACCUCUUGGUCUUUGAAUAAAGCCUGAGUAGGA
AGUCUAGAAAAAAAAAAAAAAAAAAAAAAAAAAAAAAAAAAAAAAAAAAAAA
AAAAAAAAAAAAAAAAAAAAAAAAAAAAAAAAAA [SEQ ID NO: 896]
AsCas12a mRNA #3
(5′ Modification = CleanCap ® AG 3′Ome; Uridine Modifications = N1-Methyl-Pseudo UTP;
3′ Tail Modification = 79A tail)
AGGAAAUAAGAGAGAAAAGAAGAGUAAGAAGAAAUAUAAGAGCCACCAUGAC
ACAGUUCGAGGGCUUUACCAACCUGUAUCAGGUGAGCAAGACACUGCGGUUU
GAGCUGAUCCCACAGGGCAAGACCCUGAAGCACAUCCAGGAGCAGGGCUUCAU
CGAGGAGGACAAGGCCCGCAAUGAUCACUACAAGGAGCUGAAGCCCAUCAUCG
AUCGGAUCUACAAGACCUAUGCCGACCAGUGCCUGCAGCUGGUGCAGCUGGAU
UGGGAGAACCUGAGCGCCGCCAUCGACUCCUAUAGAAAGGAGAAAACCGAGGA
GACAAGGAACGCCCUGAUCGAGGAGCAGGCCACAUAUCGCAAUGCCAUCCACG
ACUACUUCAUCGGCCGGACAGACAACCUGACCGAUGCCAUCAAUAAGAGACAC
GCCGAGAUCUACAAGGGCCUGUUCAAGGCCGAGCUGUUUAAUGGCAAGGUGC
UGAAGCAGCUGGGCACCGUGACCACAACCGAGCACGAGAACGCCCUGCUGCGG
AGCUUCGACAAGUUUACAACCUACUUCUCCGGCUUUUAUGAGAACAGGAAGA
ACGUGUUCAGCGCCGAGGAUAUCAGCACAGCCAUCCCACACCGCAUCGUGCAG
GACAACUUCCCCAAGUUUAAGGAGAAUUGUCACAUCUUCACACGCCUGAUCAC
CGCCGUGCCCAGCCUGCGGGAGCACUUUGAGAACGUGAAGAAGGCCAUCGGCA
UCUUCGUGAGCACCUCCAUCGAGGAGGUGUUUUCCUUCCCUUUUUAUAACCAG
CUGCUGACACAGACCCAGAUCGACCUGUAUAACCAGCUGCUGGGAGGAAUCUC
UCGGGAGGCAGGCACCGAGAAGAUCAAGGGCCUGAACGAGGUGCUGAAUCUG
GCCAUCCAGAAGAAUGAUGAGACAGCCCACAUCAUCGCCUCCCUGCCACACAG
AUUCAUCCCCCUGUUUAAGCAGAUCCUGUCCGAUAGGAACACCCUGUCUUUCA
UCCUGGAGGAGUUUAAGAGCGACGAGGAAGUGAUCCAGUCCUUCUGCAAGUA
CAAGACACUGCUGAGAAACGAGAACGUGCUGGAGACAGCCGAGGCCCUGUUUA
ACGAGCUGAACAGCAUCGACCUGACACACAUCUUCAUCAGCCACAAGAAGCUG
GAGACAAUCAGCAGCGCCCUGUGCGACCACUGGGAUACACUGAGGAAUGCCCU
GUAUGAGCGGAGAAUCUCCGAGCUGACAGGCAAGAUCACCAAGUCUGCCAAGG
AGAAGGUGCAGCGCAGCCUGAAGCACGAGGAUAUCAACCUGCAGGAGAUCAUC
UCUGCCGCAGGCAAGGAGCUGAGCGAGGCCUUCAAGCAGAAAACCAGCGAGAU
CCUGUCCCACGCACACGCCGCCCUGGAUCAGCCACUGCCUACAACCCUGAAGA
AGCAGGAGGAGAAGGAGAUCCUGAAGUCUCAGCUGGACAGCCUGCUGGGCCU
GUACCACCUGCUGGACUGGUUUGCCGUGGAUGAGUCCAACGAGGUGGACCCCG
AGUUCUCUGCCCGGCUGACCGGCAUCAAGCUGGAGAUGGAGCCUUCUCUGAGC
UUCUACAACAAGGCCAGAAAUUAUGCCACCAAGAAGCCCUACUCCGUGGAGAA
GUUCAAGCUGAACUUUCAGCGGCCUACACUGGCCCGUGGCUGGGACGUGAAUA
AGGAGAAGAACAAUGGCGCCAUCCUGUUUGUGAAGAACGGCCUGUACUAUCU
GGGCAUCAUGCCAAAGCAGAAGGGCAGGUAUAAGGCCCUGAGCUUCGAGCCCA
CAGAGAAAACCAGCGAGGGCUUUGAUAAGAUGUACUAUGACUACUUCCCUGA
UGCCGCCAAGAUGAUCCCAAGGUGCAGCACCCAGCUGAAGGCCGUGACAGCCC
ACUUUCAGACCCACACAACCCCCAUCCUGCUGUCCAACAAUUUCAUCGAGCCU
CUGGAGAUCACAAAGGAGAUCUACGACCUGAACAAUCCUGAGAAGGAGCCAA
AGAAGUUUCAGACAGCCUACGCCAAGAAAACCGGCGACCAGAAGGGCUACAGA
GAGGCCCUGUGCAAGUGGAUCGACUUCACAAGGGAUUUUCUGUCCAAGUAUA
CCAAGACAACCUCUAUCGAUCUGUCUAGCCUGCGGCCAUCCUCUCAGUAUAAG
GACCUGGGCGAGUACUAUGCCGAGCUGAAUCCCCUGCUGUACCACAUCAGCUU
CCAGAGAAUCGCCGAGAAGGAGAUCAUGGAUGCCGUGGAGACAGGCAAGCUG
UACCUGUUCCAGAUCUAUAACAAGGACUUUGCCAAGGGCCACCACGGCAAGCC
UAAUCUGCACACACUGUAUUGGACCGGCCUGUUUUCUCCAGAGAACCUGGCCA
AGACAAGCAUCAAGCUGAAUGGCCAGGCCGAGCUGUUCUACCGCCCUAAGUCC
AGGAUGAAGAGGAUGGCAGCCCGGCUGGGAGAGAAGAUGCUGAACAAGAAGC
UGAAGGAUCAGAAAACCCCAAUCCCCGACACCCUGUACCAGGAGCUGUACGAC
UAUGUGAAUCACAGACUGUCCCACGACCUGUCUGAUGAGGCCAGGGCCCUGCU
GCCCAACGUGAUCACCAAGGAGGUGUCUCACGAGAUCAUCAAGGAUAGGCGCU
UUACCAGCGACAAGUUCUUGUUCCACGUGCCUAUCACACUGAACUAUCAGGCC
GCCAAUUCCCCAUCUAAGUUCAACCAGAGGGUGAAUGCCUACCUGAAGGAGCA
CCCCGAGACACCUAUCAUCGGCAUCGAUCGGGGCGAGAGAAACCUGAUCUAUA
UCACAGUGAUCGACUCCACCGGCAAGAUCCUGGAGCAGCGGAGCCUGAACACC
AUCCAGCAGUUUGAUUACCAGAAGAAGCUGGACAACAGGGAGAAGGAGAGGG
UGGCAGCAAGGCAGGCCUGGUCUGUGGUGGGCACAAUCAAGGAUCUGAAGCA
GGGCUAUCUGAGCCAGGUCAUCCACGAGAUCGUGGACCUGAUGAUCCACUACC
AGGCCGUGGUGGUGCUGGAGAACCUGAAUUUCGGCUUUAAGAGCAAGAGGAC
CGGCAUCGCCGAGAAGGCCGUGUACCAGCAGUUCGAGAAGAUGCUGAUCGAUA
AGCUGAAUUGCCUGGUGCUGAAGGACUAUCCAGCAGAGAAAGUGGGAGGCGU
GCUGAACCCAUACCAGCUGACAGACCAGUUCACCUCCUUUGCCAAGAUGGGCA
CCCAGUCUGGCUUCCUGUUUUACGUGCCUGCCCCAUAUACAUCUAAGAUCGAU
CCCCUGACCGGCUUCGUGGACCCCUUCGUGUGGAAAACCAUCAAGAAUCACGA
GAGCCGCAAGCACUUCCUGGAGGGCUUCGACUUUCUGCACUACGACGUGAAAA
CCGGCGACUUCAUCCUGCACUUUAAGAUGAACAGAAAUCUGUCCUUCCAGAGG
GGCCUGCCCGGCUUUAUGCCUGCAUGGGAUAUCGUGUUCGAGAAGAACGAGAC
ACAGUUUGACGCCAAGGGCACCCCUUUCAUCGCCGGCAAGAGAAUCGUGCCAG
UGAUCGAGAAUCACAGAUUCACCGGCAGAUACCGGGACCUGUAUCCUGCCAAC
GAGCUGAUCGCCCUGCUGGAGGAGAAGGGCAUCGUGUUCAGGGAUGGCUCCA
ACAUCCUGCCAAAGCUGCUGGAGAAUGACGAUUCUCACGCCAUCGACACCAUG
GUGGCCCUGAUCCGCAGCGUGCUGCAGAUGCGGAACUCCAAUGCCGCCACAGG
CGAGGACUAUAUCAACAGCCCCGUGCGCGAUCUGAAUGGCGUGUGCUUCGACU
CCCGGUUUCAGAACCCAGAGUGGCCCAUGGACGCCGAUGCCAAUGGCGCCUAC
CACAUCGCCCUGAAGGGCCAGCUGCUGCUGAAUCACCUGAAGGAGAGCAAGGA
UCUGAAGCUGCAGAACGGCAUCUCCAAUCAGGACUGGCUGGCCUACAUCCAGG
AGCUGCGCAACGGCGGAUCCCCUGCUGCUAAACGUGUUAAGCUUGAUGGGGGU
AGCCCGGCAGCCAAGAGAGUCAAACUCGACUAGAUGGUUUAUAUUGCGGCCGC
UUAAUUAAGCUGCCUUCUGCGGGGCUUGCCUUCUGGCCAUGCCCUUCUUCUCU
CCCUUGCACCUGUACCUCUUGGUCUUUGAAUAAAGCCUGAGUAGGAAGUCUAG
AAAAAAAAAAAAAAAAAAAAAAAAAAAAAAAAAAAAAAAAAAAAAAAAAAAA
AAAAAAAAAAAAAAAAAAAAAAAAAAA [SEQ ID NO: 897]
AsCas12a mRNA #4
(5′ Modification = CleanCap ® AG 3′Ome; Uridine Modifications = N1-Methyl-Pseudo UTP;
3′ Tail Modification = 79A tail)
AGGAAAUAAGAGAGAAAAGAAGAGUAAGAAGAAAUAUAAGAGCCACCAUGAC
ACAGUUCGAGGGCUUUACCAACCUGUAUCAGGUGAGCAAGACACUGCGGUUU
GAGCUGAUCCCACAGGGCAAGACCCUGAAGCACAUCCAGGAGCAGGGCUUCAU
CGAGGAGGACAAGGCCCGCAAUGAUCACUACAAGGAGCUGAAGCCCAUCAUCG
AUCGGAUCUACAAGACCUAUGCCGACCAGUGCCUGCAGCUGGUGCAGCUGGAU
UGGGAGAACCUGAGCGCCGCCAUCGACUCCUAUAGAAAGGAGAAAACCGAGGA
GACAAGGAACGCCCUGAUCGAGGAGCAGGCCACAUAUCGCAAUGCCAUCCACG
ACUACUUCAUCGGCCGGACAGACAACCUGACCGAUGCCAUCAAUAAGAGACAC
GCCGAGAUCUACAAGGGCCUGUUCAAGGCCGAGCUGUUUAAUGGCAAGGUGC
UGAAGCAGCUGGGCACCGUGACCACAACCGAGCACGAGAACGCCCUGCUGCGG
AGCUUCGACAAGUUUACAACCUACUUCUCCGGCUUUUAUGAGAACAGGAAGA
ACGUGUUCAGCGCCGAGGAUAUCAGCACAGCCAUCCCACACCGCAUCGUGCAG
GACAACUUCCCCAAGUUUAAGGAGAAUUGUCACAUCUUCACACGCCUGAUCAC
CGCCGUGCCCAGCCUGCGGGAGCACUUUGAGAACGUGAAGAAGGCCAUCGGCA
UCUUCGUGAGCACCUCCAUCGAGGAGGUGUUUUCCUUCCCUUUUUAUAACCAG
CUGCUGACACAGACCCAGAUCGACCUGUAUAACCAGCUGCUGGGAGGAAUCUC
UCGGGAGGCAGGCACCGAGAAGAUCAAGGGCCUGAACGAGGUGCUGAAUCUG
GCCAUCCAGAAGAAUGAUGAGACAGCCCACAUCAUCGCCUCCCUGCCACACAG
AUUCAUCCCCCUGUUUAAGCAGAUCCUGUCCGAUAGGAACACCCUGUCUUUCA
UCCUGGAGGAGUUUAAGAGCGACGAGGAAGUGAUCCAGUCCUUCUGCAAGUA
CAAGACACUGCUGAGAAACGAGAACGUGCUGGAGACAGCCGAGGCCCUGUUUA
ACGAGCUGAACAGCAUCGACCUGACACACAUCUUCAUCAGCCACAAGAAGCUG
GAGACAAUCAGCAGCGCCCUGUGCGACCACUGGGAUACACUGAGGAAUGCCCU
GUAUGAGCGGAGAAUCUCCGAGCUGACAGGCAAGAUCACCAAGUCUGCCAAGG
AGAAGGUGCAGCGCAGCCUGAAGCACGAGGAUAUCAACCUGCAGGAGAUCAUC
UCUGCCGCAGGCAAGGAGCUGAGCGAGGCCUUCAAGCAGAAAACCAGCGAGAU
CCUGUCCCACGCACACGCCGCCCUGGAUCAGCCACUGCCUACAACCCUGAAGA
AGCAGGAGGAGAAGGAGAUCCUGAAGUCUCAGCUGGACAGCCUGCUGGGCCU
GUACCACCUGCUGGACUGGUUUGCCGUGGAUGAGUCCAACGAGGUGGACCCCG
AGUUCUCUGCCCGGCUGACCGGCAUCAAGCUGGAGAUGGAGCCUUCUCUGAGC
UUCUACAACAAGGCCAGAAAUUAUGCCACCAAGAAGCCCUACUCCGUGGAGAA
GUUCAAGCUGAACUUUCAGCGGCCUACACUGGCCCGUGGCUGGGACGUGAAUG
UGGAGAAGAACCGUGGCGCCAUCCUGUUUGUGAAGAACGGCCUGUACUAUCU
GGGCAUCAUGCCAAAGCAGAAGGGCAGGUAUAAGGCCCUGAGCUUCGAGCCCA
CAGAGAAAACCAGCGAGGGCUUUGAUAAGAUGUACUAUGACUACUUCCCUGA
UGCCGCCAAGAUGAUCCCAAAGUGCAGCACCCAGCUGAAGGCCGUGACAGCCC
ACUUUCAGACCCACACAACCCCCAUCCUGCUGUCCAACAAUUUCAUCGAGCCU
CUGGAGAUCACAAAGGAGAUCUACGACCUGAACAAUCCUGAGAAGGAGCCAA
AGAAGUUUCAGACAGCCUACGCCAAGAAAACCGGCGACCAGAAGGGCUACAGA
GAGGCCCUGUGCAAGUGGAUCGACUUCACAAGGGAUUUUCUGUCCAAGUAUA
CCAAGACAACCUCUAUCGAUCUGUCUAGCCUGCGGCCAUCCUCUCAGUAUAAG
GACCUGGGCGAGUACUAUGCCGAGCUGAAUCCCCUGCUGUACCACAUCAGCUU
CCAGAGAAUCGCCGAGAAGGAGAUCAUGGAUGCCGUGGAGACAGGCAAGCUG
UACCUGUUCCAGAUCUAUAACAAGGACUUUGCCAAGGGCCACCACGGCAAGCC
UAAUCUGCACACACUGUAUUGGACCGGCCUGUUUUCUCCAGAGAACCUGGCCA
AGACAAGCAUCAAGCUGAAUGGCCAGGCCGAGCUGUUCUACCGCCCUAAGUCC
AGGAUGAAGAGGAUGGCAGCCCGGCUGGGAGAGAAGAUGCUGAACAAGAAGC
UGAAGGAUCAGAAAACCCCAAUCCCCGACACCCUGUACCAGGAGCUGUACGAC
UAUGUGAAUCACAGACUGUCCCACGACCUGUCUGAUGAGGCCAGGGCCCUGCU
GCCCAACGUGAUCACCAAGGAGGUGUCUCACGAGAUCAUCAAGGAUAGGCGCU
UUACCAGCGACAAGUUCUUGUUCCACGUGCCUAUCACACUGAACUAUCAGGCC
GCCAAUUCCCCAUCUAAGUUCAACCAGAGGGUGAAUGCCUACCUGAAGGAGCA
CCCCGAGACACCUAUCAUCGGCAUCGAUCGGGGCGAGAGAAACCUGAUCUAUA
UCACAGUGAUCGACUCCACCGGCAAGAUCCUGGAGCAGCGGAGCCUGAACACC
AUCCAGCAGUUUGAUUACCAGAAGAAGCUGGACAACAGGGAGAAGGAGAGGG
UGGCAGCAAGGCAGGCCUGGUCUGUGGUGGGCACAAUCAAGGAUCUGAAGCA
GGGCUAUCUGAGCCAGGUCAUCCACGAGAUCGUGGACCUGAUGAUCCACUACC
AGGCCGUGGUGGUGCUGGAGAACCUGAAUUUCGGCUUUAAGAGCAAGAGGAC
CGGCAUCGCCGAGAAGGCCGUGUACCAGCAGUUCGAGAAGAUGCUGAUCGAUA
AGCUGAAUUGCCUGGUGCUGAAGGACUAUCCAGCAGAGAAAGUGGGAGGCGU
GCUGAACCCAUACCAGCUGACAGACCAGUUCACCUCCUUUGCCAAGAUGGGCA
CCCAGUCUGGCUUCCUGUUUUACGUGCCUGCCCCAUAUACAUCUAAGAUCGAU
CCCCUGACCGGCUUCGUGGACCCCUUCGUGUGGAAAACCAUCAAGAAUCACGA
GAGCCGCAAGCACUUCCUGGAGGGCUUCGACUUUCUGCACUACGACGUGAAAA
CCGGCGACUUCAUCCUGCACUUUAAGAUGAACAGAAAUCUGUCCUUCCAGAGG
GGCCUGCCCGGCUUUAUGCCUGCAUGGGAUAUCGUGUUCGAGAAGAACGAGAC
ACAGUUUGACGCCAAGGGCACCCCUUUCAUCGCCGGCAAGAGAAUCGUGCCAG
UGAUCGAGAAUCACAGAUUCACCGGCAGAUACCGGGACCUGUAUCCUGCCAAC
GAGCUGAUCGCCCUGCUGGAGGAGAAGGGCAUCGUGUUCAGGGAUGGCUCCA
ACAUCCUGCCAAAGCUGCUGGAGAAUGACGAUUCUCACGCCAUCGACACCAUG
GUGGCCCUGAUCCGCAGCGUGCUGCAGAUGCGGAACUCCAAUGCCGCCACAGG
CGAGGACUAUAUCAACAGCCCCGUGCGCGAUCUGAAUGGCGUGUGCUUCGACU
CCCGGUUUCAGAACCCAGAGUGGCCCAUGGACGCCGAUGCCAAUGGCGCCUAC
CACAUCGCCCUGAAGGGCCAGCUGCUGCUGAAUCACCUGAAGGAGAGCAAGGA
UCUGAAGCUGCAGAACGGCAUCUCCAAUCAGGACUGGCUGGCCUACAUCCAGG
AGCUGCGCAACGGCGGAUCCCCUGCUGCUAAACGUGUUAAGCUUGAUGGGGGU
AGCCCGGCAGCCAAGAGAGUCAAACUCGACUAGAUGGUUUAUAUUGCGGCCGC
UUAAUUAAGCUGCCUUCUGCGGGGCUUGCCUUCUGGCCAUGCCCUUCUUCUCU
CCCUUGCACCUGUACCUCUUGGUCUUUGAAUAAAGCCUGAGUAGGAAGUCUAG
AAAAAAAAAAAAAAAAAAAAAAAAAAAAAAAAAAAAAAAAAAAAAAAAAAAA
AAAAAAAAAAAAAAAAAAAAAAAAAAA [SEQ ID NO: 898]
AsCas12a mRNA #5
(5′ Modification = CleanCap ® AG 3′Ome; Uridine Modifications = N1-Methyl-Pseudo UTP;
3′ Tail Modification = 79A tail)
AGGAAAUAAGAGAGAAAAGAAGAGUAAGAAGAAAUAUAAGAGCCACCAUGAC
ACAGUUCGAGGGCUUUACCAACCUGUAUCAGGUGAGCAAGACACUGCGGUUU
GAGCUGAUCCCACAGGGCAAGACCCUGAAGCACAUCCAGGAGCAGGGCUUCAU
CGAGGAGGACAAGGCCCGCAAUGAUCACUACAAGGAGCUGAAGCCCAUCAUCG
AUCGGAUCUACAAGACCUAUGCCGACCAGUGCCUGCAGCUGGUGCAGCUGGAU
UGGGAGAACCUGAGCGCCGCCAUCGACUCCUAUAGAAAGGAGAAAACCGAGGA
GACAAGGAACGCCCUGAUCGAGGAGCAGGCCACAUAUCGCAAUGCCAUCCACG
ACUACUUCAUCGGCCGGACAGACAACCUGACCGAUGCCAUCAAUAAGAGACAC
GCCGAGAUCUACAAGGGCCUGUUCAAGGCCGAGCUGUUUAAUGGCAAGGUGC
UGAAGCAGCUGGGCACCGUGACCACAACCGAGCACGAGAACGCCCUGCUGCGG
AGCUUCGACAAGUUUACAACCUACUUCUCCGGCUUUUAUGAAAACAGGAAGA
ACGUGUUCAGCGCCGAGGAUAUCAGCACAGCCAUCCCACACCGCAUCGUGCAG
GACAACUUCCCCAAGUUUAAGGAGAAUUGUCACAUCUUCACACGCCUGAUCAC
CGCCGUGCCCAGCCUGCGGGAGCACUUUGAGAACGUGAAGAAGGCCAUCGGCA
UCUUCGUGAGCACCUCCAUCGAGGAGGUGUUUUCCUUCCCUUUUUAUAACCAG
CUGCUGACACAGACCCAGAUCGACCUGUAUAACCAGCUGCUGGGAGGAAUCUC
UCGGGAGGCAGGCACCGAGAAGAUCAAGGGCCUGAACGAGGUGCUGAAUCUG
GCCAUCCAGAAGAAUGAUGAGACAGCCCACAUCAUCGCCUCCCUGCCACACAG
AUUCAUCCCCCUGUUUAAGCAGAUCCUGUCCGAUAGGAACACCCUGUCUUUCA
UCCUGGAGGAGUUUAAGAGCGACGAGGAAGUGAUCCAGUCCUUCUGCAAGUA
CAAGACACUGCUGAGAAACGAGAACGUGCUGGAGACAGCCGAGGCCCUGUUUA
ACGAGCUGAACAGCAUCGACCUGACACACAUCUUCAUCAGCCACAAGAAGCUG
GAGACAAUCAGCAGCGCCCUGUGCGACCACUGGGAUACACUGAGGAAUGCCCU
GUAUGAGCGGAGAAUCUCCGAGCUGACAGGCAAGAUCACCAAGUCUGCCAAGG
AGAAGGUGCAGCGCAGCCUGAAGCACGAGGAUAUCAACCUGCAGGAGAUCAUC
UCUGCCGCAGGCAAGGAGCUGAGCGAGGCCUUCAAGCAGAAAACCAGCGAGAU
CCUGUCCCACGCACACGCCGCCCUGGAUCAGCCACUGCCUACAACCCUGAAGA
AGCAGGAGGAGAAGGAGAUCCUGAAGUCUCAGCUGGACAGCCUGCUGGGCCU
GUACCACCUGCUGGACUGGUUUGCCGUGGAUGAGUCCAACGAGGUGGACCCCG
AGUUCUCUGCCCGGCUGACCGGCAUCAAGCUGGAGAUGGAGCCUUCUCUGAGC
UUCUACAACAAGGCCAGAAAUUAUGCCACCAAGAAGCCCUACUCCGUGGAGAA
GUUCAAGCUGAACUUUCAGCGGCCUACACUGGCCUCUGGCUGGGACGUGAAUA
AGGAGAAGAACAAUGGCGCCAUCCUGUUUGUGAAGAACGGCCUGUACUAUCU
GGGCAUCAUGCCAAAGCAGAAGGGCAGGUAUAAGGCCCUGAGCUUCGAGCCCA
CAGAGAAAACCAGCGAGGGCUUUGAUAAGAUGUACUAUGACUACUUCCCUGA
UGCCGCCAAGAUGAUCCCAAAGUGCAGCACCCAGCUGAAGGCCGUGACAGCCC
ACUUUCAGACCCACACAACCCCCAUCCUGCUGUCCAACAAUUUCAUCGAGCCU
CUGGAGAUCACAAAGGAGAUCUACGACCUGAACAAUCCUGAGAAGGAGCCAA
AGAAGUUUCAGACAGCCUACGCCAAGAAAACCGGCGACCAGAAGGGCUACAGA
GAGGCCCUGUGCAAGUGGAUCGACUUCACAAGGGAUUUUCUGUCCAAGUAUA
CCAAGACAACCUCUAUCGAUCUGUCUAGCCUGCGGCCAUCCUCUCAGUAUAAG
GACCUGGGCGAGUACUAUGCCGAGCUGAAUCCCCUGCUGUACCACAUCAGCUU
CCAGAGAAUCGCCGAGAAGGAGAUCAUGGAUGCCGUGGAGACAGGCAAGCUG
UACCUGUUCCAGAUCUAUAACAAGGACUUUGCCAAGGGCCACCACGGCAAGCC
UAAUCUGCACACACUGUAUUGGACCGGCCUGUUUUCUCCAGAGAACCUGGCCA
AGACAAGCAUCAAGCUGAAUGGCCAGGCCGAGCUGUUCUACCGCCCUAAGUCC
AGGAUGAAGAGGAUGGCAGCUCGGCUGGGAGAGAAGAUGCUGAACAAGAAGC
UGAAGGAUCAGAAAACCCCAAUCCCCGACACCCUGUACCAGGAGCUGUACGAC
UAUGUGAAUCACAGACUGUCCCACGACCUGUCUGAUGAGGCCAGGGCCCUGCU
GCCCAACGUGAUCACCAAGGAGGUGUCUCACGAGAUCAUCAAGGAUAGGCGCU
UUACCAGCGACAAGUUCUUGUUCCACGUGCCUAUCACACUGAACUAUCAGGCC
GCCAAUUCCCCAUCUAAGUUCAACCAGAGGGUGAAUGCCUACCUGAAGGAGCA
CCCCGAGACACCUAUCAUCGGCAUCGAUCGGGGCGAGAGAAACCUGAUCUAUA
UCACAGUGAUCGACUCCACCGGCAAGAUCCUGGAGCAGCGGAGCCUGAACACC
AUCCAGCAGUUUGAUUACCAGAAGAAGCUGGACAACAGGGAGAAGGAGAGGG
UGGCAGCAAGGCAGGCCUGGUCUGUGGUGGGCACAAUCAAGGAUCUGAAGCA
GGGCUAUCUGAGCCAGGUCAUCCACGAGAUCGUGGACCUGAUGAUCCACUACC
AGGCCGUGGUGGUGCUGGAGAACCUGAAUUUCGGCUUUAAGAGCAAGAGGAC
CGGCAUCGCCGAGAAGGCCGUGUACCAGCAGUUCGAGAAGAUGCUGAUCGAUA
AGCUGAAUUGCCUGGUGCUGAAGGACUAUCCAGCAGAGAAAGUGGGAGGCGU
GCUGAACCCAUACCAGCUGACAGACCAGUUCACCUCCUUUGCCAAGAUGGGCA
CCCAGUCUGGCUUCCUGUUUUACGUGCCUGCCCCAUAUACAUCUAAGAUCGAU
CCCCUGACCGGCUUCGUGGACCCCUUCGUGUGGAAAACCAUCAAGAAUCACGA
GAGCCGCAAGCACUUCCUGGAGGGCUUCGACUUUCUGCACUACGACGUGAAAA
CCGGCGACUUCAUCCUGCACUUUAAGAUGAACAGAAAUCUGUCCUUCCAGAGG
GGCCUGCCCGGCUUUAUGCCUGCAUGGGAUAUCGUGUUCGAGAAGAACGAGAC
ACAGUUUGACGCCAAGGGCACCCCUUUCAUCGCCGGCAAGAGAAUCGUGCCAG
UGAUCGAGAAUCACAGAUUCACCGGCAGAUACCGGGACCUGUAUCCUGCCAAC
GAGCUGAUCGCCCUGCUGGAGGAGAAGGGCAUCGUGUUCAGGGAUGGCUCCA
ACAUCCUGCCAAAGCUGCUGGAGAAUGACGAUUCUCACGCCAUCGACACCAUG
GUGGCCCUGAUCCGCAGCGUGCUGCAGAUGCGGAACUCCAAUGCCGCCACAGG
CGAGGACUAUAUCAACAGCCCCGUGCGCGAUCUGAAUGGCGUGUGCUUCGACU
CCCGGUUUCAGAACCCAGAGUGGCCCAUGGACGCCGAUGCCAAUGGCGCCUAC
CACAUCGCCCUGAAGGGCCAGCUGCUGCUGAAUCACCUGAAGGAGAGCAAGGA
UCUGAAGCUGCAGAACGGCAUCUCCAAUCAGGACUGGCUGGCCUACAUCCAGG
AGCUGCGCAACGGCGGAUCCCCUGCUGCUAAACGUGUUAAGCUUGAUGGGGGU
AGCCCGGCAGCCAAGAGAGUCAAACUCGACUAGAUGGUUUAUAUUGCGGCCGC
UUAAUUAAGCUGCCUUCUGCGGGGCUUGCCUUCUGGCCAUGCCCUUCUUCUCU
CCCUUGCACCUGUACCUCUUGGUCUUUGAAUAAAGCCUGAGUAGGAAGUCUAG
AAAAAAAAAAAAAAAAAAAAAAAAAAAAAAAAAAAAAAAAAAAAAAAAAAAA
AAAAAAAAAAAAAAAAAAAAAAAAAAA [SEQ ID NO: 899]
AsCas12a-MF mRNA #6 sequence:
AGGAAAUAAGAGAGAAAAGAAGAGUAAGAAGAAAUAUAAGAGCCACCAUGAC
ACAGUUCGAGGGCUUUACCAACCUGUAUCAGGUGAGCAAGACACUGCGGUUU
GAGCUGAUCCCACAGGGCAAGACCCUGAAGCACAUCCAGGAGCAGGGCUUCAU
CGAGGAGGACAAGGCCCGCAAUGAUCACUACAAGGAGCUGAAGCCCAUCAUCG
AUCGGAUCUACAAGACCUAUGCCGACCAGUGCCUGCAGCUGGUGCAGCUGGAU
UGGGAGAACCUGAGCGCCGCCAUCGACUCCUAUAGAAAGGAGAAAACCGAGGA
GACAAGGAACGCCCUGAUCGAGGAGCAGGCCACAUAUCGCAAUGCCAUCCACG
ACUACUUCAUCGGCCGGACAGACAACCUGACCGAUGCCAUCAAUAAGAGACAC
GCCGAGAUCUACAAGGGCCUGUUCAAGGCCGAGCUGUUUAAUGGCAAGGUGC
UGAAGCAGCUGGGCACCGUGACCACAACCGAGCACGAGAACGCCCUGCUGCGG
AGCUUCGACAAGUUUACAACCUACUUCUCCGGCUUUUAUGAGAACAGGAAGA
ACGUGUUCAGCGCCGAGGAUAUCAGCACAGCCAUCCCACACCGCAUCGUGCAG
GACAACUUCCCCAAGUUUAAGGAGAAUUGUCACAUCUUCACACGCCUGAUCAC
CGCCGUGCCCAGCCUGCGGGAGCACUUUGAGAACGUGAAGAAGGCCAUCGGCA
UCUUCGUGAGCACCUCCAUCGAGGAGGUGUUUUCCUUCCCUUUUUAUAACCAG
CUGCUGACACAGACCCAGAUCGACCUGUAUAACCAGCUGCUGGGAGGAAUCUC
UCGGGAGGCAGGCACCGAGAAGAUCAAGGGCCUGAACGAGGUGCUGAAUCUG
GCCAUCCAGAAGAAUGAUGAGACAGCCCACAUCAUCGCCUCCCUGCCACACAG
AUUCAUCCCCCUGUUUAAGCAGAUCCUGUCCGAUAGGAACACCCUGUCUUUCA
UCCUGGAGGAGUUUAAGAGCGACGAGGAAGUGAUCCAGUCCUUCUGCAAGUA
CAAGACACUGCUGAGAAACGAGAACGUGCUGGAGACAGCCGAGGCCCUGUUUA
ACGAGCUGAACAGCAUCGACCUGACACACAUCUUCAUCAGCCACAAGAAGCUG
GAGACAAUCAGCAGCGCCCUGUGCGACCACUGGGAUACACUGAGGAAUGCCCU
GUAUGAGCGGAGAAUCUCCGAGCUGACAGGCAAGAUCACCAAGUCUGCCAAGG
AGAAGGUGCAGCGCAGCCUGAAGCACGAGGAUAUCAACCUGCAGGAGAUCAUC
UCUGCCGCAGGCAAGGAGCUGAGCGAGGCCUUCAAGCAGAAAACCAGCGAGAU
CCUGUCCCACGCACACGCCGCCCUGGAUCAGCCACUGCCUACAACCCUGAAGA
AGCAGGAGGAGAAGGAGAUCCUGAAGUCUCAGCUGGACAGCCUGCUGGGCCU
GUACCACCUGCUGGACUGGUUUGCCGUGGAUGAGUCCAACGAGGUGGACCCCG
AGUUCUCUGCCCGGCUGACCGGCAUCAAGCUGGAGAUGGAGCCUUCUCUGAGC
UUCUACAACAAGGCCAGAAAUUAUGCCACCAAGAAGCCCUACUCCGUGGAGAA
GUUCAAGCUGAACUUUCAGCGGCCUACACUGGCCUCUGGCUGGGACGUGAAUA
AGGAGAAGAACAAUGGCGCCAUCCUGUUUGUGAAGAACGGCCUGUACUAUCU
GGGCAUCAUGCCAAAGCAGAAGGGCAGGUAUAAGGCCCUGAGCUUCGAGCCCA
CAGAGAAAACCAGCGAGGGCUUUGAUAAGAUGUACUAUGACUACUUCCCUGA
UGCCGCCAAGAUGAUCCCAAAGUGCAGCACCCAGCUGAAGGCCGUGACAGCCC
ACUUUCAGACCCACACAACCCCCAUCCUGCUGUCCAACAAUUUCAUCGAGCCU
CUGGAGAUCACAAAGGAGAUCUACGACCUGAACAAUCCUGAGAAGGAGCCAA
AGAAGUUUCAGACAGCCUACGCCAAGAAAACCGGCGACCAGAAGGGCUACAGA
GAGGCCCUGUGCAAGUGGAUCGACUUCACAAGGGAUUUUCUGUCCAAGUAUA
CCAAGACAACCUCUAUCGAUCUGUCUAGCCUGCGGCCAUCCUCUCAGUAUAAG
GACCUGGGCGAGUACUAUGCCGAGCUGAAUCCCCUGCUGUACCACAUCAGCUU
CCAGAGAAUCGCCGAGAAGGAGAUCAUGGAUGCCGUGGAGACAGGCAAGCUG
UACCUGUUCCAGAUCUAUAACAAGGACUUUGCCAAGGGCCACCACGGCAAGCC
UAAUCUGCACACACUGUAUUGGACCGGCCUGUUUUCUCCAGAGAACCUGGCCA
AGACAAGCAUCAAGCUGAAUGGCCAGGCCGAGCUGUUCUACCGCCCUAAGUCC
AGGAUGAAGAGGAUGGCACACCGGCUGGGAGAGAAGAUGCUGAACAAGAAGC
UGAAGGAUCAGAAAACCCCAAUCCCCGACACCCUGUACCAGGAGCUGUACGAC
UAUGUGAAUCACAGACUGUCCCACGACCUGUCUGAUGAGGCCAGGGCCCUGCU
GCCCAACGUGAUCACCAAGGAGGUGUCUCACGAGAUCAUCAAGGAUAGGCGCU
UUACCAGCGACAAGUUCUUGUUCCACGUGCCUAUCACACUGAACUAUCAGGCC
GCCAAUUCCCCAUCUAAGUUCAACCAGAGGGUGAAUGCCUACCUGAAGGAGCA
CCCCGAGACACCUAUCAUCGGCAUCGAUCGGGGCGAGAGAAACCUGAUCUAUA
UCACAGUGAUCGACUCCACCGGCAAGAUCCUGGAGCAGCGGAGCCUGAACACC
AUCCAGCAGUUUGAUUACCAGAAGAAGCUGGACAACAGGGAGAAGGAGAGGG
UGGCAGCAAGGCAGGCCUGGUCUGUGGUGGGCACAAUCAAGGAUCUGAAGCA
GGGCUAUCUGAGCCAGGUCAUCCACGAGAUCGUGGACCUGAUGAUCCACUACC
AGGCCGUGGUGGUGCUGGAGAACCUGAAUUUCGGCUUUAAGAGCAAGAGGAC
CGGCAUCGCCGAGAAGGCCGUGUACCAGCAGUUCGAGAAGAUGCUGAUCGAUA
AGCUGAAUUGCCUGGUGCUGAAGGACUAUCCAGCAGAGAAAGUGGGAGGCGU
GCUGAACCCAUACCAGCUGACAGACCAGUUCACCUCCUUUGCCAAGAUGGGCA
CCCAGUCUGGCUUCCUGUUUUACGUGCCUGCCCCAUAUACAUCUAAGAUCGAU
CCCCUGACCGGCUUCGUGGACCCCUUCGUGUGGAAAACCAUCAAGAAUCACGA
GAGCCGCAAGCACUUCCUGGAGGGCUUCGACUUUCUGCACUACGACGUGAAAA
CCGGCGACUUCAUCCUGCACUUUAAGAUGAACAGAAAUCUGUCCUUCCAGAGG
GGCCUGCCCGGCUUUAUGCCUGCAUGGGAUAUCGUGUUCGAGAAGAACGAGAC
ACAGUUUGACGCCAAGGGCACCCCUUUCAUCGCCGGCAAGAGAAUCGUGCCAG
UGAUCGAGAAUCACAGAUUCACCGGCAGAUACCGGGACCUGUAUCCUGCCAAC
GAGCUGAUCGCCCUGCUGGAGGAGAAGGGCAUCGUGUUCAGGGAUGGCUCCA
ACAUCCUGCCAAAGCUGCUGGAGAAUGACGAUUCUCACGCCAUCGACACCAUG
GUGGCCCUGAUCCGCAGCGUGCUGCAGAUGCGGAACUCCAAUGCCGCCACAGG
CGAGGACUAUAUCAACAGCCCCGUGCGCGAUCUGAAUGGCGUGUGCUUCGACU
CCCGGUUUCAGAACCCAGAGUGGCCCAUGGACGCCGAUGCCAAUGGCGCCUAC
CACAUCGCCCUGAAGGGCCAGCUGCUGCUGAAUCACCUGAAGGAGAGCAAGGA
UCUGAAGCUGCAGAACGGCAUCUCCAAUCAGGACUGGCUGGCCUACAUCCAGG
AGCUGCGCAACGGUCGUAGCAGUGAUGAUGAAGCAACCGCAGAUAGCCAGCAU
GCAGCACCCAAGAAGAAGAGGAAAGUCUAAUAGUGAAUGGUUUAUAUUGCGG
CCGCUUAAUUAAGCUGCCUUCUGCGGGGCUUGCCUUCUGGCCAUGCCCUUCUU
CUCUCCCUUGCACCUGUACCUCUUGGUCUUUGAAUAAAGCCUGAGUAGGAAGA
AAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAA
AAAAAAAAAAAAAAA [SEQ ID NO: 55]

### 5.5 Functional Analysis of Candidate Molecules

Candidate RNA-guided nucleases, gRNAs, and complexes thereof, can be evaluated by standard methods known in the art. See, e.g., Cotta-Ramusino. The stability of RNP complexes can be evaluated by differential scanning fluorimetry, as described below.

### 5.5.1 Differential Scanning Fluorimetry (DSF)

The thermostability of ribonucleoprotein (RNP) complexes comprising gRNAs and RNA-guided nucleases can be measured via DSF. The DSF technique measures the thermostability of a protein, which can increase under favorable conditions such as the addition of a binding RNA molecule, e.g., a gRNA.

A DSF assay can be performed according to any suitable protocol, and can be employed in any suitable setting, including without limitation (a) testing different conditions (e.g. different stoichiometric ratios of gRNA: RNA-guided nuclease protein, different buffer solutions, etc.) to identify optimal conditions for RNP complex formation; and (b) testing modifications (e.g. chemical modifications, alterations of sequence, etc.) of an RNA-guided nuclease and/or a gRNA to identify those modifications that improve RNP complex formation or stability. One readout of a DSF assay is a shift in melting temperature of the RNP complex; a relatively high shift suggests that the RNP complex is more stable (and can thus have greater activity or more favorable kinetics of formation, kinetics of degradation, or another functional characteristic) relative to a reference RNP complex characterized by a lower shift. When the DSF assay is deployed as a screening tool, a threshold melting temperature shift can be specified, so that the output is one or more RNP complexes having a melting temperature shift at or above the threshold. For instance, the threshold can be 5-10° C. (e.g., 5° C., 6° C., 7° C., 8° C., 9° C., 10° C.) or more, and the output can be one or more RNP complexes characterized by a melting temperature shift greater than or equal to the threshold.

Two non-limiting examples of DSF assay conditions are set forth below: To determine the best solution to form RNP complexes, a fixed concentration (e.g., 2 μM) of RNA-guided nuclease (e.g., Cas9 or Cas12a) in water+10X SYPRO Orange® (Life Technologies cat #S-6650) is dispensed into a 384 well plate. An equimolar amount of gRNA diluted in solutions with varied pH and salt is then added. After incubating at room temperature for 10 minutes and brief centrifugation to remove any bubbles, a BIO-RAD® CFX384™ Real-Time System C1000 Touch™ Thermal Cycler with the BIO-RAD® CFX Manager software is used to run a gradient from 20° C. to 90° C. with a 1° C. increase in temperature every 10 seconds.

The second assay involves mixing various concentrations of gRNA with fixed a concentration (e.g., 2 μM) of RNA-guided nuclease (e.g., Cas9 or Cas12a) in optimal buffer from assay 1 above and incubating (e.g., at RT for 10′) in a 384 well plate. An equal volume of optimal buffer+10X SYPRO Orange® (Life Technologies® cat #S-6650) is added and the plate sealed with Microseal® B adhesive (MSB-1001). Following brief centrifugation to remove any bubbles, a BIO-RAD® CFX384™ Real-Time System C1000 Touch™ Thermal Cycler with the BIO-RAD® CFX Manager software is used to run a gradient from 20° C. to 90° C. with a 1° C. increase in temperature every 10 seconds.

### 6. Genome Editing Strategies

The genome editing systems described above are used, in various embodiments of the present disclosure, to generate edits in (i.e., to alter) targeted regions of DNA within or obtained from a cell. Various strategies are described herein to generate particular edits, and these strategies are generally described in terms of the desired repair outcome, the number and positioning of individual edits (e.g., SSBs or DSBs), and the target sites of such edits.

Genome editing strategies that involve the formation of SSBs or DSBs are characterized by repair outcomes including: (a) deletion of all or part of a targeted region; (b) insertion into or replacement of all or part of a targeted region; or (c) interruption of all or part of a targeted region. This grouping is not intended to be limiting, or to be binding to any particular theory or model and is offered solely for economy of presentation. Skilled artisans will appreciate that the listed outcomes are not mutually exclusive and that some repairs can result in other outcomes. The description of a particular editing strategy or method should not be understood to require a particular repair outcome unless otherwise specified.

Replacement of a targeted region in certain embodiments involves the replacement of all or part of the existing sequence within the targeted region with a homologous sequence, for instance through gene correction or gene conversion, two repair outcomes that are mediated by HDR pathways. HDR is promoted by the use of a donor template, which can be single-stranded or double stranded, as described in greater detail below. Single or double stranded templates can be exogenous, in which case they will promote gene correction, or they can be endogenous (e.g., a homologous sequence within the cellular genome), to promote gene conversion. Exogenous templates can have asymmetric overhangs (i.e., the portion of the template that is complementary to the site of the DSB can be offset in a 3′ or 5′ direction, rather than being centered within the donor template), for instance as described by Richardson et al. (Nature Biotechnology 34, 339-344 (2016), (Richardson), incorporated by reference). In instances where the template is single stranded, it can correspond to either the complementary (top) or non-complementary (bottom) strand of the targeted region.

Gene conversion and gene correction are facilitated, in some cases, by the formation of one or more nicks in or around the targeted region, as described in Ran and Cotta-Ramusino. In some cases, a dual-nickase strategy is used to form two offset SSBs that, in turn, form a single DSB having an overhang (e.g., a 5′ overhang).

Interruption and/or deletion of all or part of a targeted sequence can be achieved by a variety of repair outcomes. As one example, a sequence can be deleted by simultaneously generating two or more DSBs that flank a targeted region, which is then excised when the DSBs are repaired, as is described in Maeder for the LCA10 mutation. As another example, a sequence can be interrupted by a deletion generated by formation of a double strand break with single-stranded overhangs, followed by exonucleolytic processing of the overhangs prior to repair.

One specific subset of target sequence interruptions is mediated by the formation of an indel within the targeted sequence, where the repair outcome is typically mediated by NHEJ pathways (including Alt-NHEJ). NHEJ is referred to as an “error prone” repair pathway because of its association with indel mutations. In some cases, however, a DSB is repaired by NHEJ without alteration of the sequence around it (a so-called “perfect” or “scarless” repair); this generally requires the two ends of the DSB to be perfectly ligated. Indels, meanwhile, are thought to arise from enzymatic processing of free DNA ends before they are ligated that adds and/or removes nucleotides from either or both strands of either or both free ends.

Because the enzymatic processing of free DSB ends can be stochastic in nature, indel mutations tend to be variable, occurring along a distribution, and can be influenced by a variety of factors, including the specific target site, the cell type used, the genome editing strategy used, etc. Even so, it is possible to draw limited generalizations about indel formation: deletions formed by repair of a single DSB are most commonly in the 1-50 bp range but can reach greater than 100-200 bp. Insertions formed by repair of a single DSB tend to be shorter and often comprise short duplications of the sequence immediately surrounding the break site. However, it is possible to obtain large insertions, and in these cases, the inserted sequence has often been traced to other regions of the genome or to plasmid DNA present in the cells. Indel mutations and genome editing systems configured to produce indels are useful for interrupting target sequences, for example, when the generation of a specific final sequence is not required and/or where a frameshift mutation would be tolerated. They can also be useful in settings where particular sequences are preferred, insofar as the certain sequences desired tend to occur preferentially from the repair of an SSB or DSB at a given site. Indel mutations are also a useful tool for evaluating or screening the activity of particular genome editing systems and their components. In these and other settings, indels can be characterized by (a) their relative and absolute frequencies in the genomes of cells contacted with genome editing systems and (b) the distribution of numerical differences relative to the unedited sequence, e.g., +1, +2, 3, etc. As one example, in a lead-finding setting, multiple gRNAs can be screened to identify those gRNAs that most efficiently drive cutting at a target site based on an indel readout under controlled conditions. gRNAs that produce indels at or above a threshold frequency, or that produce a particular distribution of indels, can be selected for further study and development. Indel frequency and distribution can also be useful as a readout for evaluating different genome editing system implementations or formulations and delivery methods, for instance by keeping the gRNA constant and varying certain other reaction conditions or delivery methods.

### 6.1 Multiplex Strategies

While exemplary strategies discussed above have focused on repair outcomes mediated by single DSBs, genome editing systems according to this disclosure can also be employed to generate two or more DSBs, either in the same locus or in different loci. Strategies for editing that involve the formation of multiple DSBs, or SSBs, are described in, for instance, Cotta-Ramusino.

### 6.2 Donor Template Design

Donor template design is described in detail in the literature, for instance in Cotta-Ramusino. DNA oligomer donor templates (oligodeoxynucleotides or ODNs), which can be single stranded (ssODNs) or double-stranded (dsODNs), can be used to facilitate HDR-based repair of DSBs, and are particularly useful for introducing alterations into a target DNA sequence, inserting a new sequence into the target sequence, or replacing the target sequence altogether.

Whether single-stranded or double stranded, donor templates generally comprise regions that are homologous to regions of DNA within or near (e.g., flanking or adjoining) a target sequence to be cleaved. These homologous regions are referred to here as “homology arms,” and are illustrated schematically below:
[5′ homology arm]-[replacement sequence]-[3′ homology arm].

The homology arms can have any suitable length (including 0 nucleotides if only one homology arm is used), and 3′ and 5′ homology arms can have the same length or can differ in length. The selection of appropriate homology arm lengths can be influenced by a variety of factors, such as the desire to avoid homologies or microhomologies with certain sequences such as Alu repeats or other very common elements. For example, a 5′ homology arm can be shortened to avoid a sequence repeat element. In other embodiments, a 3′ homology arm can be shortened to avoid a sequence repeat element. In certain embodiments, both the 5′ and the 3′ homology arms can be shortened to avoid including certain sequence repeat elements. In addition, some homology arm designs can improve the efficiency of editing or increase the frequency of a desired repair outcome. For example, Richardson et al. Nature Biotechnology 34, 339-344 (2016) (Richardson), which is incorporated by reference, found that the relative asymmetry of 3′ and 5′ homology arms of single stranded donor templates influenced repair rates and/or outcomes.

Replacement sequences in donor templates have been described elsewhere, including in Cotta-Ramusino et al. A replacement sequence can be any suitable length (including zero nucleotides, where the desired repair outcome is a deletion), and typically comprises one, two, three or more sequence modifications relative to the naturally occurring sequence within a cell in which editing is desired. One exemplary sequence modification involves the alteration of the naturally occurring sequence to repair a mutation that is related to a disease or condition of which treatment is desired. Another exemplary sequence modification involves the alteration of one or more sequences that are complementary to, or code for, the PAM sequence of the RNA-guided nuclease or the targeting domain of the gRNA(s) being used to generate an SSB or DSB, to reduce or eliminate repeated cleavage of the target site after the replacement sequence has been incorporated into the target site.

Where a linear ssODN is used, it can be configured to (i) anneal to the nicked strand of the target nucleic acid, (ii) anneal to the intact strand of the target nucleic acid, (iii) anneal to the plus strand of the target nucleic acid, and/or (iv) anneal to the minus strand of the target nucleic acid. An ssODN can have any suitable length, e.g., about, at least, or no more than 150-200 nucleotides (e.g., 150, 160, 170, 180, 190, or 200 nucleotides).

It should be noted that a template nucleic acid can also be a nucleic acid vector, such as a viral genome or circular double stranded DNA, e.g., a plasmid. Nucleic acid vectors comprising donor templates can comprise other coding or non-coding elements. For example, a template nucleic acid can be delivered as part of a viral genome (e.g., in an AAV or lentiviral genome) that comprises certain genomic backbone elements (e.g., inverted terminal repeats, in the case of an AAV genome) and optionally comprises additional sequences coding for a gRNA and/or an RNA-guided nuclease. In certain embodiments, the donor template can be adjacent to, or flanked by, target sites recognized by one or more gRNAs, to facilitate the formation of free DSBs on one or both ends of the donor template that can participate in repair of corresponding SSBs or DSBs formed in cellular DNA using the same gRNAs. Exemplary nucleic acid vectors suitable for use as donor templates are described in Cotta-Ramusino.

Whatever format is used, a template nucleic acid can be designed to avoid undesirable sequences. In certain embodiments, one or both homology arms can be shortened to avoid overlap with certain sequence repeat elements, e.g., Alu repeats, LINE elements, etc.

### 6.3 Quantitative Measurements of On-Target and Off-Target Gene Editing

It should be noted that the genome editing systems of the present disclosure allow for the detection and quantitative measurement of on-target and off-target gene editing outcomes. The compositions and methods described herein can rely on the use of PCR primer sequences to amplify the genomic locus comprising the expected cut site of the RNA-guided nuclease. In some embodiments, the primers comprise an adaptor tail for use in a two-step PCR amplification process to prepare amplicon libraries for Next Generation Sequencing (NGS) analysis.

In certain embodiments, the genome editing systems or RNP complexes disclosed herein have minimal or no off-target effects. In certain embodiments, the off-target effect of a genome editing system or RNP complex is measured by Digenome-seq analysis (Kim et al., Nature Methods (2015); 12:237-243). In certain embodiments, the off-target effect of a genome editing system or RNP complex is indicated by an off-target count as measured by Digenome-seq analysis. In certain embodiments, the off-target count is measured by Digenome-seq analysis at 1000 nM of the RNP complex. In certain embodiments, the off-target count is measured by the Digenome-seq analysis at 100 nM of the RNP complex.

In certain embodiments, the off-target count as measured by Digenome-seq analysis of an RNP complex disclosed herein at 1000 nM is less than about 20, less than about 19, less than about 18, less than about 17, less than about 16, less than about 15, less than about 14, less than about 13, less than about 12, less than about 11, less than about 10, less than about 9, less than about 8, less than about 7, less than about 6, less than about 5, less than about 4, less than about 3, less than about 2, or less than about 1. In certain embodiments, the off-target count of an RNP complex disclosed herein as measured by Digenome-seq at 1000 nM is zero or is about zero. In certain embodiments, the off-target count of an RNP complex disclosed herein as measured by Digenome-seq at 100 nM is less than about 20, less than about 19, less than about 18, less than about 17, less than about 16, less than about 15, less than about 14, less than about 13, less than about 12, less than about 11, less than about 10, less than about 9, less than about 8, less than about 7, less than about 6, less than about 5, less than about 4, less than about 3, less than about 2, or less than about 1. In certain embodiments, the off-target count of an RNP complex disclosed herein as measured by Digenome-seq at 100 nM is zero or is about zero.

In some embodiments, a quantitative method of assessing the on-target and off-target sites, e.g., GUIDE-Seq, includes the integration of an exogenous double stranded oligo nucleotide (dsODN) tag into the genome. For example, Tsai et al., 2016; Tsai et al., 2014; and Tycko et., 2016, which are incorporated by reference herein in their entirety, describe compositions and methods which allow for the quantitative analysis of off-target and on-target gene editing outcomes, by the integration of a dsODN into RNA guided nuclease (RGN) induced double strand breaks (DSBs). In some embodiments, the dsODN tag is a 34 bp, blunt, 5′ phosphorylated, phosphorothioate linked polynucleotide that becomes incorporated into double stand breaks. The dsODN tag contains a priming site that allows for the amplification, sequencing, and discovery of RNG induced double strand breaks.

7. Implementation of genome editing systems: Delivery, Formulations, and Routes of Administration

As discussed above, the genome editing systems of this disclosure can be implemented in any suitable manner, meaning that the components of such systems, including without limitation the RNA-guided nuclease, gRNA, and optional donor template nucleic acid, can be delivered, formulated, or administered in any suitable form or combination of forms that results in the transduction, transfection, expression or introduction of a genome editing system and/or causes a desired repair outcome in a cell, tissue or subject. The genome editing systems according to this disclosure can incorporate multiple gRNAs, multiple RNA-guided nucleases (or polynucleotides (e.g., mRNA) encoding such RNA-guided nucleases), and other components such as proteins, and a variety of implementations will be evident to the skilled artisan based on the principles illustrated in systems of the disclosure. In some embodiments the genome editing system of the disclosure is delivered into cells as a ribonucleoprotein (RNP) complex. In some embodiments, one or more RNP complexes are delivered to the cell sequentially in any order, or simultaneously. In some embodiments the genome editing system described herein is delivered into cells (e.g., via an LNP) as a gRNA and an RNA encoding an RNA-guided nuclease protein. Tables 19 and 20 set forth several, non-limiting examples of genome editing system implementations. Those of skill in the art will appreciate, however, that these listings are not comprehensive, and that other implementations are possible. With reference to Table 19 in particular, the table lists several exemplary implementations of a genome editing system comprising a single gRNA and an optional donor template. However, genome editing systems according to this disclosure can incorporate multiple gRNAs, multiple RNA-guided nucleases, and other components such as proteins, and a variety of implementations will be evident to the skilled artisan based on the principles illustrated in the table. In the table, [N/A] indicates that the genome editing system does not comprise the indicated component.

TABLE 19
Genome Editing System Components

| RNA- | | Donor | |
|---|---|---|---|
| guided | | Tem- | |
| Nuclease | gRNA | plate | Comments |
| Protein | RNA | [N/A] | An RNA-guided nuclease protein |
| | | | complexed with a gRNA molecule (an |
| | | | RNP complex) |
| Protein | RNA | DNA | An RNP complex as described above |
| | | | plus a single-stranded or double |
| | | | stranded donor template. |
| Protein | DNA | [N/A] | An RNA-guided nuclease protein plus |
| | | | gRNA transcribed from DNA. |
| Protein | DNA | DNA | An RNA-guided nuclease protein plus |
| | | | gRNA-encoding DNA and a separate |
| | | | DNA donor template. |
| Protein | DNA | | An RNA-guided nuclease protein and a |
| | | | single DNA encoding both a gRNA and |
| | | | a donor template. |
| DNA | | | A DNA or DNA vector encoding an RNA- |
| | | | guided nuclease, a gRNA, and a donor |
| | | | template. |
| DNA | DNA | [N/A] | Two separate DNAs, or two separate DNA |
| | | | vectors, encoding the RNA-guided |
| | | | nuclease and the gRNA, respectively. |
| DNA | DNA | DNA | Three separate DNAs, or three separate |
| | | | DNA vectors, encoding the RNA-guided |
| | | | nuclease, the gRNA, and the donor |
| | | | template, respectively. |
| DNA | | [N/A] | A DNA or DNA vector encoding an RNA- |
| | | | guided nuclease and a gRNA |
| DNA | | DNA | A first DNA or DNA vector encoding an |
| | | | RNA-guided nuclease and a gRNA, and a |
| | | | second DNA or DNA vector encoding a |
| | | | donor template. |
| DNA | DNA | | A first DNA or DNA vector encoding an |
| | | | RNA-guided nuclease and second DNA or |
| | | | DNA vector encoding a gRNA and a |
| | | | donor template. |
| DNA | | | A first DNA or DNA vector encoding an |
| | DNA | | RNA-guided nuclease and a donor |
| | | | template, and a second DNA or DNA |
| | | | vector encoding a gRNA |
| DNA | | | A DNA or DNA vector encoding an RNA- |
| | RNA | | guided nuclease and a donor template, and |
| | | | a gRNA |

| RNA | [N/A] | An RNA, e.g., mRNA, or RNA vector |
|---|---|---|
| | | encoding an RNA-guided nuclease and |
| | | comprising a gRNA |
| RNA | DNA | An RNA or RNA vector encoding an |

RNA-guided nuclease and comprising a
gRNA, and a DNA or DNA vector
encoding a donor template.

Table 20 summarizes various delivery methods for the components of genome editing systems, as described herein. Again, the listing is intended to be exemplary rather than limiting.

TABLE 20
Summary of delivery methods for components of genome editing systems

| | | Delivery | | | |
|---|---|---|---|---|---|
| | | into Non- | | | Type of |
| | | Dividing | Duration of | Genome | Molecule |
| Delivery Vector/Mode | | Cells | Expression | Integration | Delivered |
| Physical (e.g., electroporation, | | YES | Transient | NO | Nucleic Acids |
| particle gun, Calcium | | | | | and Proteins |
| Phosphate transfection, cell | | | | | |
| compression or squeezing) | | | | | |
| Non-Viral | Lipid | YES | Transient | Depends on | Nucleic Acids |
| | Nanoparticles | | | what is | and Proteins |
| | (LNP) | | | delivered | |
| | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Viral | Retrovirus | NO | Stable | YES | RNA |
| | Lentivirus | YES | Stable | YES/NO with | RNA |
| | | | | modifications | |
| | Adenovirus | YES | Stable | NO | DNA |
| | Adeno- | YES | Stable | NO | DNA |
| | Associated Virus | | | | |
| | (AAV) | | | | |
| | Vaccinia Virus | YES | Very | NO | DNA |
| | | | Transient | | |
| | Herpes Simplex | YES | Stable | NO | DNA |
| | Virus | | | | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

### 7.1 Nucleic Acid-Based Delivery of Genome Editing Systems

Nucleic acids encoding the various elements of a genome editing system according to the present disclosure can be administered to subjects or delivered into cells by art-known methods or as described herein. For example, RNA-guided nuclease-encoding and/or gRNA-encoding DNA, as well as donor template nucleic acids can be delivered by, e.g., vectors (e.g., viral or non-viral vectors), non-vector-based methods (e.g., using naked DNA or DNA complexes), or a combination thereof. In some embodiments the genome editing system of the disclosure are delivered by AAV. Non-limiting examples of AAV serotypes include AAV1, AAV2, AAV3, AAV4, AAV5, AAV6, AAV7, AAV8, AAV9, AAV10, AAV11, AAV12, AAV13. In certain embodiments, the AAV serotypes can be matched to target cell types. For example, in certain non-limiting embodiments, AAV serotypes selected from the group consisting of AAV3, AAV5, AAV8, and AAV9 can be used to target liver cells.

### 7.1.1 Non-Viral Vectors

Non-viral vectors can be used to deliver nucleic acids encoding genome editing systems according to the present disclosure. One important category of non-viral nucleic acid vectors are nanoparticles, which can be organic or inorganic. Nanoparticles are well known in the art and are summarized in Cotta-Ramusino. Any suitable nanoparticle design can be used to deliver genome editing system components or nucleic acids encoding such components. For instance, organic (e.g., lipid and/or polymer) nanoparticles can be suitable for use as delivery vehicles in certain embodiments of this disclosure. In one embodiment, the vector is a lipid nanoparticle (LNP).

In certain embodiments, the LNP can have a diameter of less than 1000 nm, 500 nm, 250 nm, 200 nm, 150 nm, 100 nm, 75 nm, 50 nm, or 25 nm. In some embodiments, the LNP has a size ranging from about 1 nm to about 1000 nm. In some embodiments, the LNP has a size ranging from about 1 nm to about 500 nm. In some embodiments, the LNP has a size ranging from about 1 nm to about 250 nm. In some embodiments, the LNP has a size ranging from about 25 nm to about 200 nm. In some embodiments, the LNP has a size ranging from about 25 nm to about 100 nm. In some embodiments, the LNP has a size ranging from about 35 nm to about 75 nm. In some embodiments, the LNP has a size ranging from about 25 nm to about 60 nm.

In certain embodiments, but not by way of any limitation, LNPs can be made from ionizable lipids (e.g., cationic lipids), neutral lipids, structural lipids, helper lipids, and PEGylated lipids, or a combination of these. In some embodiments, fusogenic phospholipids (e.g., DOPE) and/or sterols (e.g., cholesterol), may be included in LNPs as ‘helper lipids’ to enhance transfection activity and/or LNP stability. Exemplary lipids and polymers for use in nanoparticle formulations and/or gene transfer are shown in Table 21, and Table 22. Further non-limiting examples of suitable lipids comprise those described in WO 2025/133951, Dec. 18, 2024, the entire contents of which are incorporated herein by reference.

TABLE 21
Lipids Used for LNPs and/or Gene Transfer

| Lipid | Abbreviation | Feature |
|---|---|---|
| ((4-Hydroxybutyl)azanediyl)bis(hexane-6,1-diyl) bis(2- | ALC-0315 | Cationic |
| hexyldecanoate) | | |
| 8-[(2-hydroxyethyl)[6-oxo-6-(undecyloxy)hexyl]amino]- | SM-102 | Cationic |
| octanoic acid, 1-octylnonyl ester | | |
| di((Z)-non-2-en-1-yl) 8,8′-((2-((2- | ATX-001 | Cationic |
| (dimethylamino)ethyl)thio)acetyl)azanediyl)dioctanoate | | |
| di((Z)-non-2-en-1-yl) 8,8′-((((2- | ATX-002 | Cationic |
| (dimethylamino)ethyl)thio)carbonyl)azanediyl)dioctanoate | | |
| (2Z)-2-Nonen-1-yl 4-[[[[2- | ATX-081 | Cationic |
| (dimethylamino)ethyl]thio]carbonyl][4-[(1-heptyloctyl)oxy]- | | |
| 4-oxobutyl]amino]butanoate | | |
| ATX-095 | ATX-095 | Cationic |
| ATX-0126 | ATX-0126 | Cationic |
| Dilinoleyl-methyl-4-dimethylaminobutyrate | D-Lin-MC3- | Cationic |
| | DMA | |
| [(6Z,16Z)-12-[(Z)-dec-4-enyl]docosa-6,16-dien-11-yl] 5- | Lipid 10 | Cationic |
| (dimethylamino)pentanoate | (CL-1) | |
| N-[1-(2,3-Dioleyloxy)propyl]N,N,N-trimethylammonium | DOTMA | Cationic |
| chloride | | |
| 1,2-Dioleoyloxy-3-trimethylammonium-propane | DOTAP | Cationic |
| 2,3-dioleyloxy-N-[2-(sperminecarboxamido)ethyl]-N,N- | DOSPA | Cationic |
| dimethyl-1-propanaminium | | |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| [(±)-N-(3-aminopropyl)-N,N-dimethyl-2,3-bis(cis-9- | GAP- | Cationic |
| tetradecenyloxy)-1-propanaminium bromide] | DMORIE | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N- | DOSPA | Cationic |
| dimethyl-1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium | DMRI | Cationic |
| bromide | | |
| 3β-[N-(N′,N′-Dimethylaminoethane)-carbamoyl]cholesterol | DC-Chol | Cationic |
| 1,2-dioleoyl-3-trimethylammonium-propane-cholesterol | DOTAP- | Cationic |
| | cholesterol | |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14- | Cationic |
| | amidine | |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | D-LinDMA | Cationic |
| 2,2-dilinoleyl-4-dimethylaminoethyl-[1,3]-dioxolane | D-Lin-KC2- | Cationic |
| | DMA | |
| 1,2-Dioleoyl-sn-glycero-3-phosphatidylethanolamine | DOPE | Helper/ |
| | | Neutral |
| Cholesterol | — | Helper/ |
| | | Neutral |
| 1,2-Diphytanoyl-sn-glycero-3-phosphatidylethanolamine | D-PyPE | Helper/ |
| | | Neutral |
| 1,2-Dioleoyl-sn-glycero-3-phosphatidylcholine | DOPC | Helper/ |
| | | Neutral |
| 1, 2-Distearoylphosphatidylcholine | DSPC | Helper/ |
| | | Neutral |
| Dipalmitoylphosphatidylcholine | DPPC | Helper/ |
| | | Neutral |
| 1-palmitoyl-2-oleoyl-glycero-3-phosphocholine | POPC | Helper/ |
| | | Neutral |

TABLE 22
Polymers Used for LNPs and/or Gene Transfer

| Polymer | Abbreviation |
|---|---|
| Poly(ethylene)glycol | PEG |
| dimyristoyl-sn-glycero-3-methoxypolyethylene glycol | DMG-PEG |
| distearoyl-sn-glycerol-3-methoxypolyethylene glycol | DSG-PEG |
| distearoyl-sn-glycero-3-phosphoethanolamine-N- | DSPE-PEG |
| methoxypolyethylene glycol | |
| Polyethylenimine | PEI |
| Dithiobis(succinimidylpropionate) | DSP |
| Dimethyl-3,3′-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

In certain embodiments, the LNPs of the present disclosure comprise a cationic lipid, e.g., those cationic lipids disclosed in Table 21. In certain embodiments, the cationic lipids that find use in the compositions and methods of the present disclosure include, but are not limited to: 1,2-dilinoleyloxy-N,N-dimethylaminopropane (DLinDMA); 1,2-dilinolenyloxyN, N-dimethylaminopropane (DLenDMA); 2,2-dilinoleyl-4-(2-dimethyl-aminoethyl)-[1,3]dioxolane (DLin-K-C2-DMA.; “ ” XTC2′ ”); 2,2-dilinoleyl-4-(3-dimethyl-aminoopropyl)-[1,3]-dioxolane (DLin-K-C3-DMA); 2,2-dilinoieyl-4-(4-dimethylaminobutyl)-11,3]-dioxolane (DLinK-C4-DMA); 2,2-dilinoleyl-5-dimethylaminomethyl-[J,3]-dioxane (DLin-K6-DMA); 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA); 1,2-dilinoleylcarbamoyl-oxy-3-dirn.ethylaminopropane (DLin-C-DAP); 1,2-dilinoleyoxy-3-(dimethylamino) acetoxy-propane (DLin-MAC); 1,2-dilinoleyoxy-3-morpholinopropane (DLin-MA); L2-dilinoleoyl-3-dimethylaminopropane (DLinDAP), 1,2-dilinoleylthio-3-dimethylaminopropane (DLin-SDMA); 1-linoleoyi-2-linoleyloxy-3-dimethylaminopropane (DLin-2-DMAP); 1,2-dilinoleyloxy-3-trirmethylaminopropane chloride salt (DLin-TMA C1), 1,2-dilinoleoyl-3-trimethylaminopropane chloride salt (DLin-TAP C1); 1,2-dilinoleyloxy-3-(N-methylpiperazino)propane (DLin-MPZ); 3-(N,N-dilinoleylamino)-1,2-propanediol (DLinAP); 3-(N,N-dioleylamino)-1,2-propanedio (DOAP); 1,2-dilinoleyloxo-3-(2-N,Ndimethylamino) ethoxypropane (DLin-EG-DMA); N,N-dioleyl-N,N-dimethylammonium chloride (DODAC); 1,2-dioleyloxy-N,N-dimethylanrinopropane (DODMA); 1,2-distearyloxyN,N-dimethylamino-propane (DSDMA); N-(1-(2,3-dioley loxy)propyl)-N,N,N-trirnethylammonium chloride (DOTMA); N,N-distearyl-N,N-dimethylammonium bromide (DDAB); N-(1-(2,3-dioleoyloxy) propyl)-N,N,N-trimethylammonium chloride (DOTAP); 3-(N—(N′,N′-dimethylamino-ethane)-carbanloyl) cholesterol (DC-Chol); N-(1,2-dirn.yristyloxyprop-3-yl)-N,N-dimethyl-N-hydroxyethyl ammonium (DMRIE); bromide 2,3-dioleyioxy-N-[2 (spermine-carboxmnido)ethyl]-N,N-dimethyl-1-propanarniniumtrifluoroacetate (DOSPA); dioctadecyl-amidoglycyl spermine (DOGS); 3-dimethylamino-2-(cholest-5-en-3-betaoxybutan-4-oxy)-1-(cis,cis-9, 12-octadecadienoxy)propane (CLinDMA); 2-[5′-(cholest-5-en-3-beta-oxy)-3′-oxapentoxy)-3-dimethy 1-1-(cis,cis-9′, 1-2′-octadecadienoxy)propane (CpLinDMA); 1,2-Dilinoleoy lcarbamy 1-3-dimethyiaminopropane (DLinCDAP); N-dimethyi-3,4-dioleyloxybenzylamine (DMOBA); and 1,2-N,N′-dioleylcarbamyl-3-dimethylaminopropane (DOcarbDAP).

In certain embodiments, the cationic lipids that find use in the compositions and methods of the present disclosure include, but are not limited to: (9Z,12Z)-3-((4,4-bis(octyloxy)butanoyl)oxy)-2-((((3-(diethylamino)propoxy)carbonyl)oxy)methyl)propyl octadeca-9,12-dienoate (also known as LP-01) or a pharmaceutically acceptable salt thereof. In certain embodiments, the cationic lipids that find use in the compositions and methods of the present disclosure include, but are not limited to: (9Z,9′Z, 12Z,12′Z)-2-(((3-(4-methylpiperazin-1-yl)propanoyl)oxy)methyl)propane-1,3-diyl bis(octadeca-9,12-dienoate); (9Z,9′Z,12Z,12′Z)-2-(((4-(pyrrolidin-1-yl)butanoyl)oxy)methyl)propane-1,3-diylbis(octadeca-9,12-dienoate); (9Z,9′Z,12Z,12′Z)-2-(((4-(piperidin-1-yl)butanoyl)oxy)methyl)propane-1,3-diyl bis(octadeca-9,12-dienoate); (9Z,9Z,12Z,2′Z)-2-(((1,4-dimethylpiperidine-4-carbonyl)oxy)methyl)propane-1,3-diyl bis(octadeca-9,12-dienoate); (9Z,9′Z,12Z, 12′Z)-2-(((1-(cyclopropylmethyl) piperidine-4-carbonyl)oxy)methyl)propane-1,3-diyl bis(octadeca-9,12-dienoate); (9Z,12Z)-3-((4,4-bis(octyloxy)butanoyl)oxy)-2-((((3-(dimethylamino)propoxy)carbonyl)oxy)methyl)propyl octadeca-9,12-dienoate; (9Z,12Z)-3-((4,4-bis(octyloxy)butanoyl)oxy)-2-(((((1-ethylpiperidin-3-yl)methoxy)carbonyl)oxy)methyl)propyl octadeca-9,12-dienoate; 2-((((2-(diethylamino)ethoxy)carbonyl)oxy)methyl)propane-1,3-diylbis(2-heptylundecanoate); (9Z,12Z)-3-(((2-(diethylamino)ethoxy)carbonyl)oxy)-2-(((2-heptylundecanoyl)oxy)methyl)propyl octadeca-9,12-dienoate; 2-((((3-(dimethylamino)propoxy)carbonyl)oxy)methyl)propane-1,3-diyl bis(2-heptylundecanoate); (9Z,12Z)-3-(((3-(diethylamino)propoxy)carbonyl)oxy)-2-(((2-heptylundecanoyl)oxy)methyl)propyl octadeca-9,12-dienoate; (9Z, 12Z)-3-(((2-(dimethylamino)ethoxy)carbonyl)oxy)-2-(((3-octylundecanoyl)oxy)methyl)propyl octadeca-9,12-dienoate; 2-((((3-(diethylamino)propoxy)carbonyl)oxy)methyl)propane-1,3-diyl bis(3-octylundecanoate); (9Z,12Z)-3-(((3-(diethylamino)propoxy)carbonyl)oxy)-2-(((3-octylundecanoyl)oxy)methyl) propyl octadeca-9,12-dienoate; (9Z,12Z)-3-(((3-(diethylamino)propoxy)carbonyl)oxy)-2-(((7-hexyltridecaonoyl)oxy)methyl)propyl octadeca-9,12-dienoate; (9Z,12Z)-3-(((3-(diethylamino)propoxy)carbonyl)oxy)-2-(((9-pentyltetradecanoyl)oxy)methyl)propyl octadeca-9,12-dienoate; (9Z,12Z)-3-(((3-(diethylamino)propoxy) cabonyl)oxy)-2-(((5-heptyldodecanoyl)oxy)methyl)propyl octadeca-9,12-dienoate; (9,12Z)-3-(2,2-bis(heptyloxy) acetoxy)-2-((((2-(dimethylamino)ethoxy)carbonyl)oxy)methyl)propyl octadeca-9,12-dienoate; (9Z,12Z)-3-((6,6-bis(octyloxy) hexanoyl)oxy)-2-((((3-(diethylamino)propoxy)carbonyl)oxy)methyl)propyl octadeca-9,12-dienoate; 2-(3-ethyl-11-(((9Z,12Z)-octadeca-9,12-dienoyloxy)methyl)-8,14-dioxo-7,9,13-trioxa-3-azaheptadecan-17-y1)propane-1,3-diyl dioctanoate; (9Z,9′Z,12Z, 12Z)-2-((((3-(dimethylamino)propoxy)carbonyl)oxy)methyl)propane-1,3-diyl bis(octadeca-9,12-dienoate); (9Z,9′Z,12Z,12′Z)-2-((((3-(diethylamino)propoxy)carbonyl)oxy)methyl)propane-1,3-diyl bis(octadeca-9,12-dienoate); (9Z,9′Z, 12Z, 12′Z)-2-((((2-(dimethylamino)ethoxy)carbonyl)oxy)methyl)propane-1,3-diyl bis(octadeca-9,12-dienoate); 3-((4,4-bis(octyloxy)butanoyl)oxy)-2-(((9Z,12Z)-octadeca-9,12-dienoyloxy)methyl)propyl 1-isopropylpiperidine-4-carboxylate; 3-((4,4-bis(octyloxy)butanoyl)oxy)-2-(((9Z,12Z)-octadeca-9,12-dienoyloxy)methyl)propyl 1-(cyclopropylmethyl) piperidine-4-carboxylate; 3-((4,4-bis(octyl oxy)butanoyl)oxy)-2-(((9Z,12Z)-octadeca-9,12-dienoyloxy)methyl)propyl 1-methylpyrrolidine-3-carboxylate; (9Z,9′Z, 12Z, 12Z)-2-((((3-(diethylamino)propoxy)carbonyl)oxy)methyl)-2-(((9Z,12Z)-octadeca-9,12-dienoyloxy)methyl)propane-1,3-diyl bis(octadeca-9,12-dienoate); (2S)-3-((4,4-bis(octyloxy)butanoyl)oxy)-2-(((9Z,12Z)-octadeca-9,12-dienoyloxy)methyl)propyl 1-methylpyrrolidine-2-carboxylate; (2R)-3-((4,4-bis(octyloxy)butanoyl)oxy)-2-(((9Z,12Z)-octadeca-9,12-dienoyloxy)methyl)propyl 1-methylpyrrolidine-2-carboxylate; 3-((4,4-bis(octyloxy)butanoyl)oxy)-2-(((9Z,12Z)-octadeca-9,12-dienoyloxy)methyl)propyl 4-methylmorpholine-2-carboxylate; 3-((4,4-bis(octyloxy)butanoyl)oxy)-2-(((9Z,12Z)-octadeca-9,12-di enoyl oxy)methyl)propyl 1,4-dimethylpiperidine-4-carboxylate; 3-((4,4-bis(octyloxy)butanoyl)oxy)-2-(((9Z,12Z)-octadeca-9,12-dienoyloxy)methyl)propyl 1-methylpiperidine-4-carboxylate; (9Z,12Z)-3-((4,4-bis(octyloxy)butanoyl)oxy)-2-(((((1-methylpyrrolidin-3-yl)oxy)carbonyl)oxy)methyl)propyl octadeca-9,12-dienoate; (9Z,12Z)-3-((4,4-bis(octyloxy)butanoyl)oxy)-2-(((((1-methylpiperidin-4-yl)oxy)carbonyl)oxy)methyl)propyl octadeca-9,12-dienoate; (9Z,12Z)-3-((4,4-bis(octyloxy)butanoyl)oxy)-2-(((((1-methylazetidin-3-yl)oxy)carbonyl)oxy)methyl)propyl octadeca-9,12-dienoate; (9Z,12Z)-3-((4,4-bis(octyloxy)butanoyl)oxy)-2-(((((1-ethylpiperidin-4-yl)oxy)carbonyl)oxy)methyl)propyl octadeca-9,12-dienoate; (9Z,12Z)-3-((4,4-bis(octyloxy)butanoyl)oxy)-2-(((((1-methylpiperidin-4-yl)methoxy)carbonyl)oxy)methyl)propyl octadeca-9,12-dienoate; (9Z,12Z)-3-((4,4-bis(octyloxy)butanoyl)oxy)-2-(((((1,2,2,6,6-pentamethylpiperidin-4-yl)oxy)carbonyl)oxy)methyl)propyl octadeca-9,12-dienoate; (9Z,12Z)-3-((4,4-bis(octyloxy)butanoyl)oxy)-2-((((3-(dimethylamino)propyl)carbamoyl)oxy)methyl)propyl octadeca-9,12-dienoate; 3-((4,4-bis((2-propylpentyl)oxy)butanoyl)oxy)-2-(((9Z,12Z)-octadeca-9,12-dienoyloxy)methyl)propyl 1,4-dimethylpiperidine-4-carboxylate; 3-((6,6-bis((2-propylpentyl)oxy) hexanoyl)oxy)-2-(((9Z,12Z)-octadeca-9,12-dienoyloxy)methyl)propyl 1,4-dimethylpiperidine-4-carboxylate; 2-(((1-methylpyrrolidine-3-carbonyl)oxy)methyl)propane-1,3-diyl bis(4,4-bis(octyloxy)butanoate); 2-(((1-methylpyrrolidine-3-carbonyl)oxy)methyl)propane-1,3-diyl bis(6,6-bis(octyloxy) hexanoate); 2-(((1-methylpyrrolidine-3-carbonyl)oxy)methyl)propane-1,3-diyl bis(6,6-bis((2-propylpentyl)oxy) hexanoate); 2-(5-(3-(dodecanoyloxy)-2-(((1-methylpyrrolidine-3-carbonyl)oxy)methyl)propoxy)-5-oxopentyl)propane-1,3-diyl dioctanoate; 2-(5-(3-((1-methylpyrrolidine-3-carbonyl)oxy)-2-((palmitoyloxy)methyl)propoxy)-5-oxopentyl)propane-1,3-diyl dioctanoate; 2-(S-(3-((1-methylpyrrolidine-3-carbonyl)oxy)-2-((tetradecanoyloxy)methyl)propoxy)-S-oxopentyl)propane-1,3-diyl dioctanoate; 3-((4,4-bis(octyl oxy)butanoyl)oxy)-2-((dodecanoyloxy)methyl)propyl 1-methylpyrrolidine-3-carboxylate; 3-((4,4-bis(octyloxy)butanoyl)oxy)-2-((tetradecanoyloxy)methyl)propyl 1-methylpyrrolidine-3-3-((4,4-bis(octyloxy)butanoyl)oxy)-2-((palmitoyloxy)methyl)propyl 1-methylpyrrolidine-3-carboxylate; 1-(3-((4,4-bis(octyloxy)butanoyl)oxy)-2-(((1-methylpyrrolidine-3-carbonyl)oxy)methyl)propyl) 8-methyl octanedioate; 1-(3-((4,4-bis(octyloxy)butanoyl)oxy)-2-(((1-methylpyrrolidine-3-carbonyl)oxy)methyl)propyl) 10-octyl decanedioate; 1-(3-((6,6-bis((2-propylpentyl)oxy) hexanoyl)oxy)-2-(((1,4-dimethylpiperidine-4 carbonyl)oxy)methyl)propyl) 10-octyl decanedioate; 1-(3-((6,6-bis((2-propylpentyl)oxy) hexanoyl)oxy)-2-(((1,4-dimethylpiperidine-4-carbonyl)oxy)methyl)propyl) 8-methyl octanedioate; and 8-dimethyl O′1,01-(2-(((1-methylpyrrolidine-3-carbonyl)oxy)methyl)propane-1,3-diyl)dioctanedioate.

In certain embodiments, the cationic lipids that find use in the compositions and methods of the present disclosure include, but are not limited to: ((4,4′-((((3-(Dimethylamino)propyl)thio)carbonyl)azanediyl)bis(butanoyl))bis(oxy))bis(propane-2,1,3-triyl)tetranonanoate; ((4,4′-((((3-(Dimethylamino)propyl)thio)carbonyl)azanediyl)bis(butanoyl))bis(oxy))bis(propane-2,1,3-triyl)tetraoctanoate; bis(1,3-bis(Nonanoyloxy)propan-2-yl) 5-((4-(dimethylamino)butanoyl)oxy) nonanedioate HCl salt; bis(1,3-bis(Octanoyloxy) propan-2-yl) 5-((4-(dimethylamino)butanoyl)thio) nonanedioate; bis(1,3-bis(N onanoyloxy) propan-2-yl) 4-((4-(dimethylamino)butanoyl)oxy)heptanedioate; bis(1,3-bis(Octanoyloxy) propan-2-yl) 4-((4-(dimethylamino)butanoyl)thio)heptanedioate; ((2,2′-((((3-(Dimethyl-amino)propyl)thio)carbonyl)azanediyl)bis(acetyl))bis(oxy))bis(propane-2,1,3-triyl)tetranon-anoate; bis(1,3-bis(N onanoyloxy)propan-2-yl) 4-((4-(dimethylamino)butanoyl)thio) heptanedioate; ((4,4′-((((3-(Dimethylamino)propyl)thio)carbonyl)azanediyl)bis(butanoyl))bis(oxy))bis(propane-2,1,3-triyl)tetrakis(3-cyclohexylpropanoate); ((4,4′-((((3-(Dimethyl-amino)propyl)thio)carbonyl)azanediyl)bis(butanoyl))bis(oxy))bis(propane-2,1,3-triyl)tetrakis(4-cyclohexyl butanoate); ((6,6′-((((3-(Dimethylamino)propyl)thio)carbonyl)azanedi-yl)bis(hexanoyl))bis(oxy))bis(propane-2,1,3-triyl)tetrakis(3-cyclohexylpropanoate); Nonanoic acid 2-(3- {(3-dimethylaminopropylsulfanylcarbonyl)-[2-(2-nonanoyloxy-1-nonanoyloxymethyl-ethoxycarbonyl)-ethyl]amino}-propionyloxy)-3-octanoyloxy-propyl ester; ((4,4′-((((3-(Dimethylamino)propyl)thio)carbonyl)azanediyl)bis(butanoyl))bis(oxy))bis(propane-2,1,3-triyl)tetrakis(2-(4-methylcyclohexyl)acetate); ((4,4′-((((3-(Dimethylamino)propyl)thio)carbonyl)azanediyl)bis(butanoyl))bis(oxy))bis(propane-2,1,3-triyl)tetrakis(4-ethylcyclo hexane-1-carboxylate); ((4,4′-((((3-(Dimethylamino) propyl)thio)carbonyl)azanediyl)bis(butanoyl))bis(oxy))bis(propane-2,1,3-triyl)tetrakis(3-cyclohexyl-2-methylpropanoate); ((4,4′-((((3-(Dimethylamino)propyl)thio)carbonyl) azanediyl)bis(butanoyl))bis(oxy))bis(propane-2,1,3-triyl)tetrakis(2-methyloctanoate); ((4,4′-((((3-(Dimethylamino)propyl)thio)carbonyl)azanediyl)bis(butanoyl))bis(oxy))bis(propane-2, 1,3-triyl)tetrakis(2,2-dimethylheptanoate); ((3,3′-((((3-(Dimethylamino) propyl)thio)carbonyl)azanediyl)bis(propanoyl))bis(oxy))bis(methylene))bis(2-methylpropane-2,1,3-triyl)tetrakis(3-(4-methyl cyclohexyl)propanoate); ((3,3′-((((3-(Dimethylamino)propyl)thio)carbonyl)azanediyl)bis(propanoyl))bis(oxy))bis(methylene))bis(2-methyl propane-2, 1,3-triyl)tetrakis(2-(4-ethylcyclohexyl)acetate); ((3,3′-((((3-(Dimethylamino)propyl)thio)carbonyl)azanediyl)bis(propanoyl))bis(oxy))bis(methylene))bis(2-methyl propane-2, 1,3-triyl)tetrakis(2-(4-ethylcyclohexyl)acetate); ((3,3′-((((3-(Dimethylamino)propyl)thio)carbonyl)azanediyl)bis(propanoyl))bis(oxy))bis(methylene))bis(2-methylpropane-2,1,3-triyl)tetrakis(3,3-dimethylheptanoate); ((3,3′-((((3-(Dimethylamino) propyl)thio)carbonyl)azanediyl)bis(propanoyl))bis(oxy))bis(methyl-ene))bis(2-methyl-propane-2,1,3-triyl)tetrakis(octanoate); ((4,4′-((((3-(Dimethylamino)propyl)thio)carbonyl) azanediyl)bis(butanoyl))bis(oxy))bis(methylene))bis(propane-2,1,3-triyl)tetranonanoate; ((3,3′-((((2-(Dimethylamino)ethyl)thio)carbonyl)azanediyl)bis(propanoyl))bis(oxy))bis(methylene))bis(propane-2,1,3-triyl)tetranonanoate; ((3,3′-((((3-(Dimethylamino)propyl)thio)carbonyl)azanediyl)bis(propanoyl))bis(oxy))bis(methylene))bis(propane-2,1,3-triyl)tetrakis(3-(4-methylcyclohexyl)propanoate); ((4,4′-((((3-(Dimethyl-amino)propyl)thio) carbonyl)azanediyl)bis(butanoyl))bis(oxy)bis(propane-2,1,3-triyl)tetrakis(octahydro-1H-indene); ((4,4′-((((3-(dimethylamino)propyl)thio)carbonyl)azanediyl)bis(butanoyl))bis(oxy)) bis(propane-2,1,3-triyl)tetrakis(octahydro-1H-indene-5-carboxylate); ((4,4′-(((3-(dimethyl-amino)propoxy)carbonyl)azanediyl)bis(butanoyl))bis(oxy))bis(propane-2,1,3-triyl)tetranonanoate; ((4,4′-(((3-(dimethylamino)propyl)carbamoyl)azanediyl)bis(butanoyl))bis(oxy))bis(propane-2,1,3-triyl)tetranonanoate; ((4,4′-((((3-(dimethylamino)propyl)thio) carbonyl)azanediyl)bis(butanoyl))bis(oxy))bis(propane-2,1,3-triyl)tetrakis(2-(p-tolyl)acetate); [2-[4-[3-(dimethylamino)propoxycarbonyl-[4-[2-(2-methyloctanoyloxy)-1-(2-methyloctanoyloxymethyl)ethoxy]-4-oxobutyl]amino]butanoyloxy]-3-(2-methyloctanoyl-oxy)propyl] 2-methyloctanoate; [2-[4-[3-(dimethylamino)propylcarbamoyl-[4-[2-(2-methyloctanoyl oxy)-1-(2-methyloctanoyloxymethyl)ethoxy]-4-oxobutyl]amino]butanoyl-oxy]-3-(2-methyloctanoyloxy)propyl] 2-methyloctanoate; [2-[4-[[3-(dimethylamino)propyl-methyl-carbamoyl][4-[2-(2-methyloctanoyloxy)-1-(2-methyloctanoyloxymethyl)ethoxy]-4-oxobutyl]amino]butanoyloxy]-3-(2-methyloctanoyloxy)propyl] 2-methyloctanoate; [2-[4-[5-(dimethylamino) pentanoyl-[4-[2-(2-methyloctanoyloxy)-1-(2-methyloctanoyloxym ethyl)ethoxy]-4-oxo-butyl]amino]butanoyloxy]-3-(2-methyloctanoyloxy)propyl] 2-methyloctanoate; [2-[4-[5-(dimethylamino) pentyl-[4-[2-(2-methyl octanoyloxy)-1-(2-methyl-octanoyloxym ethyl)ethoxy]-4-oxo-butyl]amino]butanoyloxy]-3-(2-methyloctanoyl-oxy)propyl] 2-methyloctanoate; bis [2-(2-methyloctanoyloxy)-1-(2-methyloctanoyl oxym ethyl)ethyl] 5-[4-(dimethylamino)butanoyloxy] nonanedioate; and ((4,4′-((((3-(dimethylamino)propyl)thio)carbonyl)azanediyl)bis(butanoyl))bis(oxy))bis(propane-2, 1,3-triyl)tetrakis(2-methyl-4-(p-tolyl)butanoate).

In certain embodiments, the cationic lipids that find use in the compositions and methods of the present disclosure include, but are not limited to: 6,6′-(Methylazanediyl)Bis(N,N-Dioctylhexanamide); 6,6′-(Octylazanediyl)Bis(N,N-Dioctylhexan-amide); 6,6′-(Hexylazanediyl)Bis(N,N-Dioctylhexanamide); 10, 10′-(Methylazanediyl)Bis(N,N-Dioctyldecanamide); 8,8′-(Methylazanediyl)Bis(N,N-Didecyloctanamide); 6,6′-(Methylazanediyl)Bis(N,N-Didecylhexanamide); 6,6′-(Methylazanediyl)Bis(N,N-Didodecyl-hexanamide); 6,6′-((2-Hydroxyethyl)Azanediyl)Bis(N,N-Dioctylhexanamide); 6,6′-((6-Hydroxyhexyl)Azanediyl)Bis(N,N-Dioctylhexanamide); 6,6′-((2-Hydroxyethyl)Azanediyl)Bis(N,N-Didecylhexanamide); 8,8′-((6-Hydroxyhexyl)Azanediyl)Bis(N,N-Dioctyloct-anamide); 10,10′-((6-Hydroxyhexyl)Azanediyl)Bis(N,N-Dioctyldecanamide); 10,10′-((2-Hydroxyethyl)Azanediyl)Bis(N,N-Didecyldecanamide); 8,8′-((5-Hydroxypentyl)Azanediyl)Bis(N,N-Didecyloctanamide); 8,8′-((4-Hydroxybutyl)Azanediyl)Bis(N,N-Didecyloct-anamide); 8,8′-((6-Hydroxyhexyl)Azanediyl)Bis(N,N-Didecyloctanamide); 8,8′-((2-Hydroxy-ethyl)Azanediyl)Bis(N,N-Didecyloctanamide); 10,10′-((4-Hydroxybutyl)Azanediyl)Bis(N,N-Didecyldecanamide); N,N′-((Methylazanediyl)Bis(Hexane-6, 1-Diyl))Bis(N,2-Dihexyldecanamide); N,N′-((Methylazanediyl)Bis(Hexane-6, 1-Diyl))Bis(N-Hexyl Palmitamide); (9z,9′z, 12z, 12′z)—N,N′-((Methylazanediyl)Bis(Hexane-6,L-Diyl))Bis(N-Hexyloctadeca-9,L2-Dienamide); N,N-Didecyl-8-((8-(Hexadecylamino)-8-Oxooctyl)(Methyl)Amino) Octanamide; 8,8′-((8-(Decylamino)-8-Oxooctyl)Azanediyl)Bis(N,N-Didecyloctanamide); 8,8′-((6-(Dihexylamino)-6-Oxohexyl)Azanediyl)Bis(N,N-Didecyloctan-amide); 8,8′-((5-(Decylamino)-5-Oxopentyl)Azanediyl)Bis(N,N-Didecyloctanamide); 6,6′-((8-(Decylamino)-8-Oxooctyl)Azanediyl)Bis(N,N-Didecylhexanamide); 6,6′-((2-(Dihexylamino)Ethyl)Azanediyl)Bis(N,N-Didecylhexanamide); 10,10′-((2-(Dimethyl-amino)Ethyl)Azanediyl)Bis(N,N-Didecyldecanamide); 2-Butyloctyl 6-(Bis(6-(Dioctylamino)-6-Oxohexyl)Amino) Hexanamide; 6,6′-((4-Hydroxybutyl)Azanediyl)Bis(N,N-Bis(2-Ethylhexyl) Hexanamide); 8,8′-((2-Hydroxyethyl)Azanediyl)Bis(N,N-Didodecyl-octanamide); 6,6′-((6-Hydroxyhexyl)Azanediyl)Bis(N,N-Didodecylhexanamide); N,N-Didecyl-8-((8-(Hexadecyl(Methyl)Amino)-8-Oxooctyl)(Methyl)Amino) Octanamide; 8,8′-(Methylazanediyl)Bis(N,N-Didodecyloctanamide); 8,8′-((3-Hydroxypropyl)Azanediyl)Bis(N,N-Didecyloctanamide); 8,8′-((2-(2-Hydroxyethoxy)Ethyl)Azanediyl)Bis(N,N-Didecyloctanamide); 8,8′-((5-Hydroxy-4,4-Dimethylpentyl)Azanediyl)Bis(N,N-Didecyl-octanamide); 8,8′-((3-(2-Methyl-Lh-Imidazol-L-Yl)Propyl)Azanediyl)Bis(N,N-Didecyloctan-amide); 8,8′-((7-Hydroxyheptyl)Azanediyl)Bis(N,N-Didecyloctanamide); 8,8′-((2-(2-Methoxyethoxy)Ethyl)Azanediyl)Bis(N,N-Didecyloctanamide); 8,8′-((8-Hydroxyoctyl) Azanediyl)Bis(N,N-Didecyloctanamide); 8,8′-((3-(Lh-Imidazol-L-Yl)Propyl)Azanediyl)Bis(N,N-Didecyloctanamide); 8,8′-((2,2-Difluoro-3-Hydroxypropyl)Azanediyl)Bis(N,N-Didecyloctanamide); 8,8′-((3-((2-(Methylamino)-3,4-Dioxocyclobut-L-En-Ly1)Amino) Propyl)Azanediyl)Bis(N,N-Didecyloctanamide); 8,8′-((2-Fluoro-3-Hydroxypropyl) Azanediyl)Bis(N,N-Didecyloctanamide); 8,8′-((3,3,3-Trifluoro-2-(Hydroxymethyl)Propyl) Azanediyl)Bis(N,Ndidecyloctanamide); 8,8′-((5-Methoxypentyl)Azanediyl)Bis(N,N-Didecyloctanamide); N,N-Didecyl-8-((8-(Dioctylamino)-8-Oxooctyl)(Methyl)Amino) Octanamide; Tert-Butyl(3-(Bis(10-(Didecylamino)-10-oxodecyl)Amino)Propyl)Carbamate; 10,10′-((3-(Lh-Imidazol-L-Y1)Propyl)Azanediyl)Bis(N,N-Didecyldecanamide); 8,8′-(Methylazanediyl)Bis(N,N-Dinonyloctanamide); Tert-Butyl(3-(Bis(10-(Didecylamino)-10-oxodecyl)Amino)Propyl)Carbamate; 10,10′-((3-((2-(Methylamino)-3,4-Dioxocyclobut-L-En-Lyl)Amino)Propyl)Azanediyl)Bis(N,N-Didecyldecanamide); N,N′-((methylazanediyl)bis(octane-8, 1-diyl))bis(N-hexylhexanamide); N,N′-(((5-hydroxypentyl)azanediyl)bis(octane-8, 1-diyl))bis(N-hexylhexanamide); N,N′-((methylazanediyl)bis(octane-8, 1-diyl))bis(N-octyloctanamide); N,N′-(((5-hydroxypentyl)azanediyl)bis(octane-8, 1-diyl))bis(N-octyloctanamide); N,N′-((methylazanediyl)bis(octane-8, 1-diyl))bis(N-octyloctanamide); N,N′-(((5-hydroxypentyl)azanediyl)bis(octane-8,1-diyl))bis(N-decyldecanamide); N,N′-((methylazanediyl)bis(octane-8, 1-diyl))bis(N-dodecyldodecanamide); N,N′-(((5-hydroxypentyl)azanediyl)bis(octane-8, 1-diyl))bis(Ndodecyldodecanamide); N,N′-((methylazanediyl)bis(octane-8, 1-diyl))bis(2-hexyldecanamide); N,N′-((methylazanediyl)bis(octane-8, 1-diyl))bis(2-hexyl-N-methyldecanamide); N,N′-(((5-hydroxypentyl)azanediyl)bis(octane-8, 1-diyl))bis(2-hexyldecanamide); N,N′-(((5-hydroxypentyl)azanediyl)bis(octane-8, 1-diyl))bis(2-hexyl-N methyldecanamide); N-decyl-N-(8-((8-(didecylamino)-8 oxooctyl)(methyl)amino) octyl) decanamide; N-decyl-N-(8-((8-(didecylamino)-8-oxooctyl) (5-hydroxypentyl)amino) octyl) decanamide; 6,6′-((5-hydroxypentyl)azanediyl)bis(N,N-didecyl-hexanamide); 7, 7′-(methylazanediyl)bis(N,N-didecylheptanamide); 8,8′-((2-(dimethylamino)ethyl) azanediyl)bis(N,N-didecyloctanamide); 8,8′-((2-(pyrrolidin-1-yl)ethyl)azanediyl)bis(N,N-didecyloctanamide); N,N-didecyl-8-((8-(decyl(methyl)amino)-8-oxooctyl)(methyl)amino) octanamide; N,N-didecyl-8-((8-(decyl(methyl)amino)-8-oxooctyl) (2-hydroxyethyl)amino) octanamide; 8,8′-((5-Hydroxypentyl)Azanediyl)Bis(N,N-Dinonyloctanamide); 8,8′-((5-Hydroxypentyl)Azanediyl)Bis(N,N-Didecyl-2-Fluorooctanamide); 8,8′-(Methyl-azanediyl)Bis(N,N-Didecyl-2-Fluorooctanamide); 2,2′-((5-Hydroxypentyl)Azanediyl)Bis(N,N-Didecylacetamide); and 4,4′-((5-Hydroxypentyl)Azanediyl)Bis(N,N-Didecylbutan-amide).

Moreover, each of the following references, which disclose ionizable cationic lipids and other lipid components that find use in connection with the compositions and methods disclosed herein, is hereby incorporated by reference in its entirety: international patent application publications WO2015/095340, WO2022/011156, WO2022/133344, WO2023/086514, WO2023/081776, WO2023/010128, WO2022/235972, WO2022/235935, WO2022/235923, WO2022/056413, WO2022/036170, WO2020/191103, WO2018/183901, WO2018/119163, WO2015074085A1, WO2016081029A1, WO2017117530A1, WO2019191780A, WO2020/097548, WO2020/097540, WO2020/097520, WO2020/097493, WO2016/197133, WO2011/141705, WO2011/141704, WO2011/000107, WO2011/000106, WO2010/144740, WO2010/129709,WO2010/088537, WO2010/054406, WO2010/054405, WO2010/054401, WO2010/054384, WO2009/127060, WO2008/042973, WO2007/012191, WO2006/074546, WO2005/121348, WO2005/120461, WO2005/120152, WO2005/026372, WO2005/007196, WO2004/002453, WO2002/087541, WO2000/003683, WO2023/114944, WO2023/114943, WO2023/114937, WO2022/016070, WO2021/030701, WO2020/146805, WO2020/081938, WO2020/061426, WO2019/089828, WO2019/036030, WO2019/036028, WO2019/036008, WO2019/036000, WO2018/200943, WO2018/191719, WO2018/191657, WO2018/081480, WO2018/078053, WO2017/117528, WO2017/075531, WO2017/004143, WO2015/199952; WO2013/126803, WO2025/133951, US patent numbers U.S. Pat. Nos. 10,227,302, 10,383,952, and 10,526,284; and US application publication US2020/0297634.

In certain embodiments, the LNPs of the present disclosure comprise a non-cationic lipid, e.g., those non-cationic lipids disclosed in Table 21. In certain embodiments, the non-cationic lipids that find use in the compositions and methods of the present disclosure include, but are not limited to lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetyl-phosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoyl-phosphatidylglycerol (DPPG), dioleoyl phosphatidyl ethanol amine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-lcarboxylate (DOPE-mal), dipalmitoyl-phosphatidyl-ethanolamine (DPPE), dimyristoylphosphatidylethanolamine (DMPE), distearoyl-phosphatidylethanolamine (DSPE), monomethyl-phosphatidyl ethanol amine, phosphatidyl-ethanolamine (DEPE), dimethyl-phosphatidylethanolamine, dielaidoylstearoyloleoyl-phosphatidylethanolamine (SOPE).

In certain embodiments, the PEGylated lipids comprise a PEG molecule with a molecular weight from about 200 Da to about 5000 Da. In certain embodiments, the PEGylated lipids comprise a PEG molecule with a molecular weight of 2000 Da (2 kDa).

In certain embodiments, the PEG-lipids can include, but are not limited to, those identified in Table 22. For example, but not by way of limitation, The PEG-lipid can comprise PEG coupled to dialkyloxypropyls (PEG-DAA), PEG coupled to diacylglycerol (PEG-DAG), methoxypolyethyleneglycol (PEG-DMG or PEG2000-DMG), PEG coupled to phospholipids such as phosphatidylethanolamine (PEG-PE), PEG conjugated to ceramides, PEG conjugated to cholesterol or a derivative thereof, and mixtures thereof. In certain embodiments, the PEG-lipid comprises detergent-like PEG lipids (e.g., PEG-DSPE).

In certain embodiments, the PEG moiety is conjugated directly to the lipid. In certain embodiments, the PEG moiety is conjugated to the lipid via a linker moiety. Any linker moiety suitable for conjugating the PEG to a lipid can be used including, but not limited to, ester-containing linker moieties and/or non-ester-containing linker moieties. In certain embodiments, an ester-containing linker moiety is used to conjugate the PEG to the lipid. Exemplary ester-containing linker moieties include, e.g., carbonate (—OC(O)O—), succinoyl, phosphate esters (—O—(O)POH—O—), sulfonate esters, and combinations thereof. Exemplary non-ester containing linker moieties include, but are not limited to, amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH₂CH₂C(O)—), succinamidyl (—NHC(O)CH₂CH₂C(O)NH—), ether, as well as combinations thereof (such as a linker containing both a carbamate linker moiety and an amido linker moiety).

In certain embodiments, phosphatidylethanolamines having a variety of acyl chain groups of varying chain lengths and degrees of saturation can be conjugated to PEG to form the PEG-lipid conjugate. In certain embodiments, phosphatidylethanolamines comprising saturated or unsaturated fatty acids with carbon chain lengths in the range of C10 to C20 are employed in connection with the compositions and methods disclosed herein. In certain embodiments, phosphatidylethanolamines with mono- or di-unsaturated fatty acids and mixtures of saturated and unsaturated fatty acids can also be used. In certain embodiments, the phosphatidylethanolamines that find use in connection with the compositions and methods disclosed herein include, but are not limited to, dimyristoyl-phosphatidylethanolamine (DMPE), dipalmitoyl-phosphatidylethanolamine (DPPE), dioleoylphosphatidylethanolamine (DOPE), and distearoyl-phosphatidylethanolamine (DSPE).

In certain embodiments the PEG-DAA conjugate of the instant disclosure is a PEG-didecyloxypropyl (C10) conjugate, a PEG-dilauryloxypropyl (C12) conjugate, a PEG-dimyristyloxypropyl (C14) conjugate, a PEG-dipalmityloxypropyl (C16) conjugate, or a PEG-distearyloxypropyl (C18) conjugate. In certain of such embodiments, the PEG moiety has an average molecular weight of about 750 or about 2,000 daltons. In certain of such embodiments, the terminal hydroxyl group of the PEG moiety is substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxy propyl, methacrylamide, polymethacrylamide, and polydimethylacrylamide, polylactic acid, polyglycolic acid, and derivatized celluloses such as hydroxymethylcellulose or hydroxyethylcellulose.

The lipids described herein can be combined in any number of molar ratios to produce an LNP. In certain embodiments, the LNP comprises a PEG-lipid where the PEG-lipid comprises at least 0.1 mol % of the total lipid. For example, but not by way of limitation, the PEG-lipid component can comprise from about 0.1 mol % to 5 mol % of the total lipid. In certain embodiments, the LNP comprises a cationic lipid where the cationic lipid component comprises at least 10 mol % of the total lipid. For example, but not by way of limitation, the cationic lipid component can comprise from about 10 mol % to 70 mol % of the total lipid. In certain embodiments, the cationic lipid component can comprise from about 10 mol % to 60 mol % of the total lipid. In certain embodiments, the cationic lipid component can comprise from about 10 mol % to 50 mol % of the total lipid. In certain embodiments, the cationic lipid component can comprise from about 10 mol % to 40 mol % of the total lipid. In certain embodiments, the cationic lipid component can comprise from about 10 mol % to 30 mol % of the total lipid, the cationic lipid component can comprise from about 10 mol % to 20 mol % of the total lipid. In certain embodiments, the LNP comprises cholesterol where cholesterol comprises at least 10 mol % of the total lipid. For example, but not by way of limitation, cholesterol can comprise from about 10 mol % to 70 mol % of the total lipid. In certain embodiments, the cholesterol can comprise from about 10 mol % to 60 mol % of the total lipid. In certain embodiments, the cholesterol can comprise from about 10 mol % to 50 mol % of the total lipid. In certain embodiments, the cholesterol can comprise from about 10 mol % to 40 mol % of the total lipid. In certain embodiments, the cholesterol can comprise from about 10 mol % to 30 mol % of the total lipid, the cholesterol can comprise from about 10 mol % to 20 mol % of the total lipid. In certain embodiments, the LNP comprises a non-cationic lipid where the non-cationic lipid comprises at least 10 mol % of the total lipid. For example, but not by way of limitation, the non-cationic lipid can comprise from about 10 mol % to 70 mol % of the total lipid. In certain embodiments, the non-cationic lipid component can comprise from about 10 mol % to 60 mol % of the total lipid. In certain embodiments, the non-cationic lipid component can comprise from about 10 mol % to 50 mol % of the total lipid. In certain embodiments, the non-cationic lipid component can comprise from about 10 mol % to 40 mol % of the total lipid. In certain embodiments, the non-cationic lipid component can comprise from about 10 mol % to 30 mol % of the total lipid, the non-cationic lipid component can comprise from about 10 mol % to 20 mol % of the total lipid.

In some embodiments, non-viral vectors include targeting modifications to improve uptake and/or selectively target certain cell types. These targeting modifications can include e.g., cell specific antigens, monoclonal antibodies, single chain antibodies, aptamers, polymers, sugars (e.g., N-acetylgalactosamine (GalNAc)), and cell penetrating peptides. Such vectors also optionally use fusogenic and endosome-destabilizing peptides/polymers, undergo acid-triggered conformational changes (e.g., to accelerate endosomal escape of the cargo), and/or incorporate a stimuli-cleavable polymer, e.g., for release in a cellular compartment. For example, disulfide-based cationic polymers that are cleaved in the reducing cellular environment can be used.

In certain embodiments, one or more nucleic acid molecules (e.g., DNA molecules) other than the components of a genome editing system, e.g., the RNA-guided nuclease component (or a polynucleotide encoding the RNA-guided nuclease component) and/or the gRNA component described herein, are delivered. In certain embodiments, the nucleic acid molecule is delivered at the same time as one or more of the components of the genome editing system. In certain embodiments, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the genome editing system are delivered. In certain embodiments, the nucleic acid molecule is delivered by a different means than one or more of the components of the genome editing system, e.g., the RNA-guided nuclease component (or a polynucleotide encoding the RNA-guided nuclease component) and/or the gRNA component, are delivered. The nucleic acid molecule can be delivered by any of the delivery methods described herein. For example, the nucleic acid molecule can be delivered by a viral vector, e.g., an AAV or an integration-deficient lentivirus, and the RNA-guided nuclease molecule component (or a polynucleotide encoding the RNA-guided nuclease component) and/or the gRNA component can be delivered by electroporation, e.g., such that the toxicity caused by nucleic acids (e.g., DNAs) can be reduced. In certain embodiments, the nucleic acid molecule encodes a therapeutic protein, e.g., a protein described herein. In certain embodiments, the nucleic acid molecule comprises an RNA molecule, e.g., an RNA molecule described herein.

The present disclosure provides lipid nanoparticle (LNP) compositions. In some embodiments, the LNP compositions comprise one or more targeting moieties. In some embodiments, the targeting moiety binds to an asialoglycoprotein receptor (ASGPR). In some embodiments, the LNP composition targets a genome editing system to a particular cell, e.g., a liver cell, e.g., a hepatocyte. In some embodiments, the targeting moiety comprises one or more N-acetylgalactosamine (GalNAc) or GalNAc derivatives. In some embodiments, the targeting moiety comprises at least two GalNAcs or GalNAc derivatives. In some embodiments, the targeting moiety comprises at least three GalNAcs or GalNAc derivatives. In some embodiments, the LNP comprises a lipid linked to the targeting moiety through a spacer and/or through one or more linkers, including, e.g., a bivalent or trivalent branched linker. Further non-limiting examples of suitable targeting moieties comprise those described in WO 2017/177326 published Oct. 19, 2017, U.S. Pat. No. 11,801,306 issued Oct. 31, 2023, WO2024220807 published Oct. 24, 2024, and Kasiewicz et al., Nat Commun. 2023 May 15; 14:2776, the entire contents of each of which are incorporated herein by reference.

In certain embodiments, the LNP comprises a PEG-lipid linked to the targeting moiety. In certain embodiments, the PEG-lipids can include, but are not limited to, those identified in Table 22. In certain embodiments, the PEG-lipid is linked to one or more GalNAc or GalNAc derivatives.

The linkers can each independently comprise one or more functional groups selected from the group consisting of ethylene glycol, propylene glycol, amide, ester, ether, alkyl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl, wherein each of the carboxyl, carbamoyl, ether, alkoxy, aryloxy, amino, amide, carbamate, alkyl, alkenyl, alkynyl, aryl, arylalkyl, cycloalkyl, heteroaryl, or heterocyclyl is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl. In some embodiments, a linker independently comprises phosphate, phosphorothioate, amide, ether, oxime, hydrazine or carbamate.

In some embodiments, the LNP compositions comprise a GalNAc-based lipid that is or comprises one or more N-acetylgalactosamine (GalNAc) or GalNAc derivatives, an ionizable lipid, a polyethylene glycol (PEG) lipid, a helper lipid, a sterol, or any combination thereof.

Another aspect of the present disclosure provides methods for delivering nucleic acids encoding the various elements of a genome editing system into a target cell comprising: contacting the target cell with or introducing into the target cell at least one lipid nanoparticle comprising a GalNAc-based lipid that comprises one or more N-acetylgalactosamine (GalNAc) or GalNAc derivatives and one or more polynucleotides.

Another aspect of the present disclosure provides a method for delivering a nucleic acid encoding the various elements of a genome editing system into a target cell comprising: contacting the target cell with or introducing into the target cell at least one lipid nanoparticle comprising a GalNAc-based lipid that comprises one or more N-acetylgalactosamine (GalNAc) or GalNAc derivatives and one or more components of a genome editing system.

The present disclosure provides methods for delivering polynucleotides into a cell comprising: contacting the target cell with or introducing into the cell at least one lipid nanoparticle comprising a GalNAc-based lipid that comprises one or more N-acetylgalactosamine (GalNAc) or GalNAc derivatives and one or more polynucleotides. In some embodiments, the polynucleotides are DNA or RNA. The present disclosure provides methods for delivering a genome editing system into a cell comprising: introducing into the cell at least one lipid nanoparticle comprising a GalNAc-based lipid that comprises one or more N-acetylgalactosamine (GalNAc) or GalNAc derivatives and one or more components of a genome editing system.

### 7.1.2 Naked Nucleic Acids

Nucleic acids encoding genome editing systems or components thereof can be delivered directly to cells as naked DNA or RNA, for instance by means of transfection or electroporation, or can be conjugated to molecules (e.g., N-acetylgalactosamine) promoting uptake by the target cells (e.g., liver cells). Nucleic acid vectors, such as the vectors summarized in Table 20, can also be used. In some embodiments the genome editing system of the disclosure is delivered into cells by electroporation.

### 7.1.3 Shuttle Vectors

One approach for cell therapy processes includes the direct delivery of active proteins into human cells. A protein delivery agent, the Feldan Shuttle, is a protein-based delivery agent, which is designed for cell therapy (Del′Guidice et al., PloSOne. 2018 Apr. 4; 13 (4): e0195558; incorporated in its entirety herein by reference). In some embodiments the genome editing system of the disclosure are delivered into cells by the Feldan Shuttle.

7.1.4 Nucleic acid vectors

Nucleic acid vectors can comprise one or more sequences encoding genome editing system components, such as an RNA-guided nuclease, a gRNA and/or a donor template. A vector can also comprise a sequence encoding a signal peptide (e.g., for nuclear localization, nucleolar localization, or mitochondrial localization), associated with (e.g., inserted into or fused to) a sequence coding for a protein. As one example, a nucleic acid vector can include an RNA-guided nuclease (e.g., Cas9 or Cas12a) coding sequence that encodes one or more nuclear localization sequences (e.g., a nuclear localization sequence from SV40).

The nucleic acid vector can also include any suitable number of regulatory/control elements, e.g., promoters, enhancers, introns, polyadenylation signals, Kozak consensus sequences, or internal ribosome entry sites (IRES). These elements are well known in the art and are described in Cotta-Ramusino.

Nucleic acid vectors according to this disclosure include recombinant viral vectors. Exemplary viral vectors are set forth in Table 20, and additional suitable viral vectors and their use and production are described in Cotta-Ramusino. Other viral vectors known in the art can also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

7.2 Delivery of RNPs and/or RNA Encoding Genome Editing System Components

RNPs (complexes of gRNAs and RNA-guided nucleases) and/or RNAs encoding RNA-guided nucleases and/or gRNAs, can be delivered into cells or administered to subjects by art-known methods, some of which are described in Cotta-Ramusino. In vitro, RNA-guided nuclease-encoding and/or gRNA-encoding RNA can be delivered, e.g., by microinjection, electroporation, transient cell compression or squeezing (see, e.g., Lee 2012). Lipid-mediated transfection, peptide-mediated delivery, GalNAc- or other conjugate-mediated delivery, and combinations thereof, can also be used for delivery in vitro and in vivo.

In vitro, delivery via electroporation comprises mixing the cells with the RNA encoding RNA-guided nucleases and/or gRNAs, with or without donor template nucleic acid molecules, in a cartridge, chamber or cuvette and applying one or more electrical impulses of defined duration and amplitude. Systems and protocols for electroporation are known in the art, and any suitable electroporation tool and/or protocol can be used in connection with the various embodiments of this disclosure.

In certain embodiments, a genome editing system or ribonucleoprotein (RNP) complex, may comprise guide RNAs, a nuclease, e.g. Cas9 or Cas12a proteins, including modified Cas12a proteins (e.g., AsCas12a variants). Non-limiting examples of Cas9 and Cas12a (Cpf1) proteins are set forth in herein. In certain embodiments, a genome editing system or RNP complex may include a guide RNA (gRNA) complexed to a Cas12a protein or a modified Cas12a protein. In certain embodiments, a genome editing system or RNP complex may include a guide RNA (gRNA) complexed to a Cas9 protein or a modified Cas9 protein. In certain embodiments a gRNA may comprise a sequence set forth in herein. In certain embodiments, a genome editing system or RNP complex may comprise an RNP complex set forth in Table 23.

For example, a genome editing system or RNP complex may include a gRNA comprising a sequence set forth in SEQ ID NOs: 900-1316 or 1331-1337, a modified Cas9 protein set forth in SEQ ID NO: 1339 and target an LDLR gene at the sequence set forth in SEQ ID NO: 61-304, 1317, or 1318. In another example, a genome editing system or RNP complex may include a gRNA comprising a sequence set forth in SEQ ID NOs: 900-903, 909-912, 958, 962, 1027, 1114, 1116, 1119, 1121-1127, 1130-1132, 1135, 1138-1140, a modified Cas9 protein set forth in SEQ ID NO: 1139, and target an LDLR gene at the sequence set forth in SEQ ID NO: 61-64, 70-73, 1119, 123, 188, 275, 277, 280, 282-288, 291-293, 296, 299-301.

In certain embodiments, a genome editing system comprises guide RNA and mRNA encoding the RNA-guided nuclease (Cas9 proteins, including modified Cas9 proteins e.g., SpCas9 variants). Non-limiting examples of mRNAs encoding the RNA-guided nuclease are set forth in SEQ ID NOs: 1349-1359. Following delivery of the guide RNA and mRNA to the cell, the mRNA drives translation of the RNA-guided nuclease, leading to formation of the RNP complexes comprising a guide RNA (gRNA) and a Cas9 protein or a modified Cas9 protein. In certain embodiments, a gRNA can comprise a sequence set forth in SEQ ID NOs: 900-1316 or 1331-1337. In certain embodiments, a genome editing system or RNP complex can comprise an RNP complex set forth in Table 23. For example, but not by way of limitation, a genome editing system or RNP complex can comprise a gRNA comprising a sequence set forth in SEQ ID NOs: 900-903, 909-912, 958, 962, 1027, 1114, 1116, 1119, 1121-1127, 1130-1132, 1135, 1138-1140, an mRNA encoding a modified Cas9 protein as set forth in SEQ ID NO: 1349 and target an LDLR gene at the sequence set forth in SEQ ID NO: 61-64, 70-73, 1119, 123, 188, 275, 277, 280, 282-288, 291-293, 296, 299-301. Additionally or alternatively, a genome editing system or RNP complex can comprise a gRNA comprising a sequence set forth in SEQ ID NOs: 900-913, 1114-1138, and 1139-1141, a modified SpCas9 protein set forth in SEQ ID NO: 1339, and target an LDLR gene at the sequence set forth in SEQ ID NO: SEQ ID NOs: 61-74, 275-298, and 300-302.

TABLE 23
Exemplary ribonucleoprotein (RNP) Configuration

| Component | Details |
|---|---|
| RNP | Complex of SpCas9 protein and gRNA that targets the site |
| | set forth in [SEQ ID NO: 61] |
| SpCas9 | SpCas9 protein variant; C-terminal linker and nuclear |
| protein | localization sequence added with sequence set forth |
| | in [SEQ ID NO: 1340] |
| gRNA | RNA oligonucleotide with the scaffold sequence set forth in |
| | [SEQ ID NO: 30] and targeting domain set forth in [SEQ |
| | ID NO: 305] |
| LDLR | 21-base pair site of LDLR, corresponding to the sequence |
| target site | set forth in [SEQ ID NO: 61] |

### 7.3 Editing Efficiency

In some embodiments, editing the LDLR gene using the genome editing systems, the RNP complexes, or the LNP delivery systems described in this disclosure increases LDLR expression by at least about 1.05-fold to about 2-fold when compared to control non-edited cells. In some embodiments, the LDLR expression level is increased by about at least 1.05-fold, 1.10-fold, 1.15-fold, 1.20-fold, 1.25-fold, 1.30-fold, 1.35-fold, 1.40-fold, 1.45-fold, 1.50-fold, 1.55-fold, 1.60-fold, 1.65-fold, 1.70-fold, 1.75-fold, 1.80-fold, 1.85-fold, 1.90-fold, 1.95-fold, or 2.0-fold compared to control cells. In some embodiments, the LDLR expression levels are increased by at least about 1.5-fold to at least about 2-fold compared to, e.g., control cells or an unedited population of cells comprising the mutation.

### 7.4 Target Cells

The genome editing systems, the RNP complexes, or the delivery systems described in this disclosure can be used to manipulate or alter a target cell, e.g., to edit or alter a target nucleic acid. In some embodiments, the genome editing system, the RNP complex, or the delivery system described in this disclosure, are used to edit target cells in a tissue, e.g., edit or alter a target nucleic acid in cells that make up the tissue. The manipulating can occur, in various embodiments, in vivo or ex vivo. In certain embodiments, the target cells are cells involved in metabolism. For example, in one embodiment, the target cells are liver cells (e.g., hepatocytes, hepatic stellate cells, a Kupffer cells, or liver stem cells). In one embodiment the target cell is a hepatocyte. In certain embodiments, the target cell comprises a genomic edit that results in upregulation of LDLR. While not wishing to be bound by any particular theory, it is contemplated that increasing or upregulating LDLR reduces the risk of major adverse cardiovascular events (MACE) including, heart attack, stroke, aortic stenosis, peripheral vascular disease, and renal dysfunction. In some embodiments, increasing or upregulating LDLR expression reduces hypercholesterolemia.

In some embodiments, editing the LDLR gene in the liver cells using the genome editing system, the RNP complex, or the delivery system described in this disclosure increases LDLR expression levels by at least about 1.05-fold to about 2-fold when compared to a control, non-edited liver cells. In some embodiments, the LDLR expression level is increased by about at least 1.05-fold, 1.10-fold, 1.15-fold, 1.20-fold, 1.25-fold, 1.30-fold, 1.35-fold, 1.40-fold, 1.45-fold, 1.50-fold, 1.55-fold, 1.60-fold, 1.65-fold, 1.70-fold, 1.75-fold, 1.80-fold, 1.85-fold, 1.90-fold, 1.95-fold, or 2.0-fold compared to control cells. In some embodiments, the LDLR expression levels are increased by at least 1.5-fold compared to control cells. In some embodiments, the LDLR expression levels are increased by at least 1.8-fold compared to control cells. In some embodiments, the LDLR expression levels are increased by at least 1.85-fold compared to control cells. In some embodiments, the LDLR expression levels are increased by at least 1.9-fold compared to control cells. In some embodiments, the LDLR expression levels are increased by at least 1.95-fold compared to control cells. In some embodiments, the LDLR expression levels are increased by at least about 2-fold compared to control cells.

In some embodiments, editing the LDLR gene in a subject (e.g., in liver cells of the subject) by administering the genome editing system, the RNP complex, or the delivery system to the subject increases an LDLR level in the subject, or in a cell, tissue, or fluid in the subject, by at least about 1.05-fold to about 2-fold relative to the LDLR level prior to administration, or relative to the LDLR level in a control (e.g., non-edited) subject, cell, tissue, or fluid. In some embodiments, the LDLR level (e.g., cell surface level assessed by flow cytometry) is increased by about at least 1.05-fold, 1.10-fold, 1.15-fold, 1.20-fold, 1.25-fold, 1.30-fold, 1.35-fold, 1.40-fold, 1.45-fold, 1.50-fold, 1.55-fold, 1.60-fold, 1.65-fold, 1.70-fold, 1.75-fold, 1.80-fold, 1.85-fold, 1.90-fold, 1.95-fold, or 2.0-fold. In some embodiments, the LDLR level (e.g., cell surface level level) is increased by at least 1.5-fold. In some embodiments, the LDLR level (e.g., cell surface level) is increased by at least 1.8-fold. In some embodiments, the LDLR level (e.g., cell surface level) is increased by at least 1.85-fold. In some embodiments, the LDLR level (e.g., cell surface level) is increased by at least 1.9-fold. In some embodiments, the LDLR level (e.g., cell surface level) is increased by at least 1.95-fold. In some embodiments, the LDLR level (e.g., cell surface level) is increased by at least 2-fold.

Provided herein are methods of administering the genome editing system, the RNP complex, or the delivery system described in this disclosure, to treat, prevent or reduce the risk of diseases, conditions, and disorders. Non-limiting examples of such disease include heterozygous familial hypercholesterolemia (HeFH), homozygous familial hypercholesterolemia (HoFH), hyperlipidemia, hypercholesterolemia atherosclerotic cardiovascular disease (ASCVD), heart attack, stroke, aortic stenosis, high blood pressure, peripheral vascular disease, diabetes, renal dysfunction, or a combination of these. In some embodiments, the genome editing system, the RNP complex, or the delivery system described in this disclosure is administered to a subject or patient having the particular disease or condition to be treated. In some aspects, the methods thereby treat, e.g., ameliorate one or more symptom of, the disease or condition.

In certain embodiments, increasing the LDLR expression level in a cell from the subject reduces the serum or plasma level of LDL and/or LDL-C.

In some embodiments, administering the genome editing system, the RNP complex, or the delivery system described in this disclosure reduces LDL levels in the subject or patient to 60 mg/dL and about 100 mg/dL, between about 70 mg/dL and about 100 mg/dL, between about 80 mg/dL and about 100 mg/dL, between about 90 mg/dL and about 100 mg/dL, between about 50 mg/dL and about 80 mg/dL, between about 30 mg/dL and about 50 mg/dL, between about 40 mg/dL and about 55 mg/dL, between about 40 mg/dL and about 60 mg/dL, between about 50 mg/dL and about 60 mg/dL, or between about 60 mg/dL and about 75 mg/dL. In some embodiments, the LDL level in the subject is measured at about 1, 2, 3, 4, 5, 6, 7, 8, 12, 16, or 20 weeks, about 1, 2, 3, 4, 5, or 6 months, or about 1, 2, 3, 4, or 5 years after administering the genome editing system, the RNP complex, or the delivery system described in this disclosure.

The genome editing system, the RNP complex, or the delivery system can be administered by any suitable means, for example, by bolus infusion, by injection, e.g., intravenous, or subcutaneous injections. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration. In some embodiments, it is administered by multiple bolus administrations, for example, over a period of no more than 3 days, or by continuous infusion administration.

### 7.5 Cell Systems and Animal Models

Evaluation of the molecules and systems described herein may include studies in multiple in vitro and in vivo systems. Primary human hepatocytes may be used for most in vitro pharmacology assessments, while HepG2 cells may be used to evaluate the functionality of specific edits by creating clones. In vivo studies may be conducted in various mouse models and NHPs (cynomolgus monkeys). Ldlr^{+/-} mice (along with WT controls) may be used for in vivo assessments to characterize the effect of various levels of editing on LDLR expression, LDL-C levels, and the effects of pharmacodynamic activity on liver function and safety endpoints over time (including a 6-month toxicity study) in the context of a patient-relevant genotype. Because of their similar physiology to humans, the NHP is considered the most relevant species for evaluation of PK/PD relationships, biodistribution, and acute toxicités associated with LNP dosing, including tolerability of novel lipid components.

A summary of cell systems and animal models that may be used to evaluate the gRNA molecules and systems described herein is provided below.

| | | |
|---|---|---|
| | LDLR | |
| Model | Status | Relevance and Justification |

In vitro

| | | |
|---|---|---|
| Primary Human | WT | In vitro model used to evaluate the effect of different edit locations on |
| Hepatocytes | | LDLR upregulation and to evaluate genotoxicity |
| (PHH) | | |
| Primary CD4⁺ | WT | In vitro model used to identify potential off-targets in a non-target cell |
| T Cells | | type by performing GUIDE-Seq using RNPs |
| Adrenal Cortical | WT | Primary human cells identified as non-target cells for genotoxicity |
| Cells | | assessment |
| Splenocytes | WT | Primary human cells identified as non-target cells for genotoxicity |
| | | assessment |
| HepG2 cells | WT | In vitro model used to evaluate the types of edits that lead to |
| | | upregulation. HepG2 cells were chosen for their hepatic origin and |
| | | ability to proliferate which enabled clonal isolation and expansion, |
| | | supplementing the PHH model. |

In vivo

| | | |
|---|---|---|
| Ldlr^{+/-} mice | Het | In vivo patient-relevant model (heterozygous loss of LDLR function) |
| | LOF | representing HeFH genotype, enabling evaluation of the relationship |
| | | between editing, LDLR upregulation, LDL-C lowering, and the safety |
| | | of chronic LDLR upregulation in a rodent model containing a single |
| | | WT allele |
| C57BL/6J mice | WT | In vivo rodent model used to evaluate PK and PD and understand the |
| | | effect of gene number on the editing response. This is the parent strain |
| | | of the Ldlr^{+/-} mice. |
| CD1 mice | WT | In vivo rodent model used to evaluate tolerability of drug product |
| | | batches prior to NHP work. Commonly used toxicology species. |
| Cynomolgus | WT | A pharmacologically relevant non-human primate (NHP) model with |
| Monkeys | | two functional LDLR genes used for evaluation of PK/PD relationships, |
| (Macaca | | biodistribution, and safety. NHPs have a similar lipid metabolism |
| fascicularis) | | mechanisms compared to humans and is a well established |
| | | pharmacology model for LDL-C lowering assessment (Tremblay et al., |
| | | Nat Med. 2025 April; 31(4): 1329-1338, doi: 10.1038/s41591-025- |
| | | 03508-x. Epub 2025 February 10, Musunuru et al., Nature 2021 |
| | | May; 593(7859): 429-434, doi: 10.1038/s41586-021-03534- |
| | | y. Epub 2021 May 19). Hence, NHP data can inform on dose |
| | | selection for pharmacological lowering of LDL-C levels in the clinic. |

Abbreviations: CD4 = Cluster of differentiation 4; Het LOF = heterozygous loss of function; LDL-C = low density lipoprotein cholesterol; LDLR = low density lipoprotein receptor; NHP = non-human primate; PD = pharmacodynamic(s); PK = pharmacokinetic(s); PHH = primary human hepatocyte; RNP = ribonucleic protein; WT = wild type.

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Those skilled in the art will recognize or be able to ascertain using no more than routine experimentation, many equivalents to the specific embodiments described herein. Such equivalents are intended to be encompassed by the following claims.

### 8. Examples

The following Examples are merely illustrative and are not intended to limit the scope or content of the invention in any way.

### 8.1 General Methods

### 8.1.1 Guide Synthesis

Guide-RNAs (gRNAs) were synthesized via standard phosphoramidite chemistry. Purification was completed by ion-pair reversed-phase preparative HPLC, followed by desalting and sequence analysis.

### 8.1.2 Lipid Nanoparticle (LNP) Formulation and Quality Control

LNPs were formulated with commercially available lipids using a NanoAssemblr® Ignite™ (CYTIVA®). Specifically, LNPs were formulated at a ratio of 50 ALC0315: 38.5 Cholesterol: 10 Distearoylphosphatidylcholine (DSPC): 1.5 Polyethylene glycol lipid. LNP cargo was mRNA encoding AsCas12a or SpCas9 nuclease and gRNA at a 1:1 ratio by weight for LNPs with single gRNAs or a 1:1:1 ratio by weight for LNPs with dual gRNAs. LNPs were evaluated for percent encapsulation greater than 80% by RiboGreen assay (ThermoFisher Scientific™), polydispersity index (PDI)<0.2, and average diameter size <105 nm by Zetasizer analysis (Malvern Panalytical, Model ZSU3205).

### 8.1.3 Cell Culture Treatments

Cells were treated with LNPs at indicated concentrations of encapsulated nuclease mRNA or nuclease mRNA and gRNA, and gDNA was isolated at 72 hours post transfection. Transfection of primary human hepatocytes (PHHs) included recombinant human Apolipoprotein E (ApoE).

### 8.2 Example 1: LDLR Guide Identification

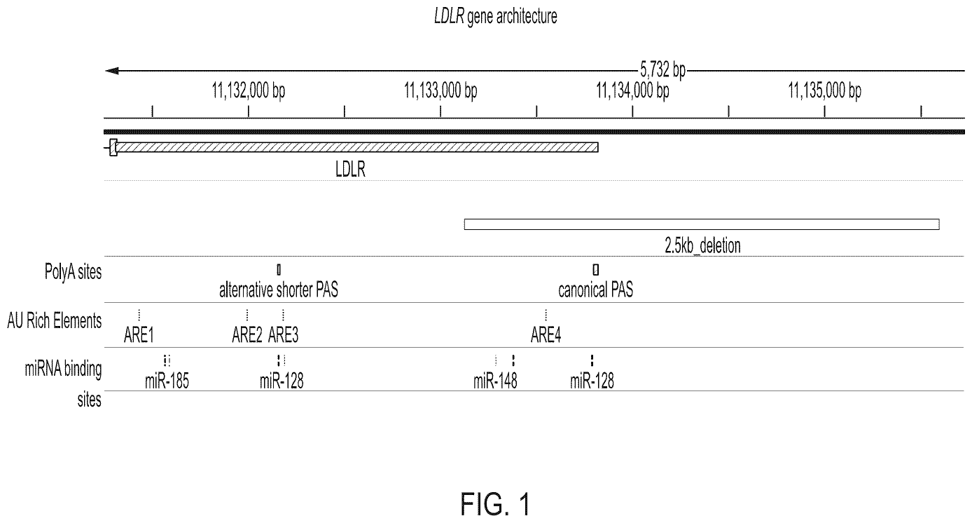

Guide RNAs were designed in silico to target the 3′ untranslated region (UTR) of the LDLR gene. FIG. 1 provides the architecture of the LDLR gene, showing the 2.5 kb deletion present in the Icelandic mutation and regulatory elements, such as the PolyA sites, AU-rich elements (AREs), and miRNA binding sites. AREs in particular have been shown to affect mRNA stability, typically promoting mRNA decay. Three AREs have been identified in the LDLR 3′ UTR, and are numbered according to their position moving from 5′ to 3′. For further discussion of the regulatory elements of the LDLR 3′ UTR, see, e.g., Li et al., J Lip Res. 2009 May; 50 (5): 820-831, Smith et al., Sci Transl Med. 2022 Sep. 14; 14 (662): eabj8670, and Wang et al., PLoS One. 2020 May 21; 15 (5): e0225356) each incorporated in their entireties herein by reference.

Guide RNAs with off targets as determined by CALITAS were eliminated. Guide RNAs that cross react with non-human primates (NHPs) were prioritized.

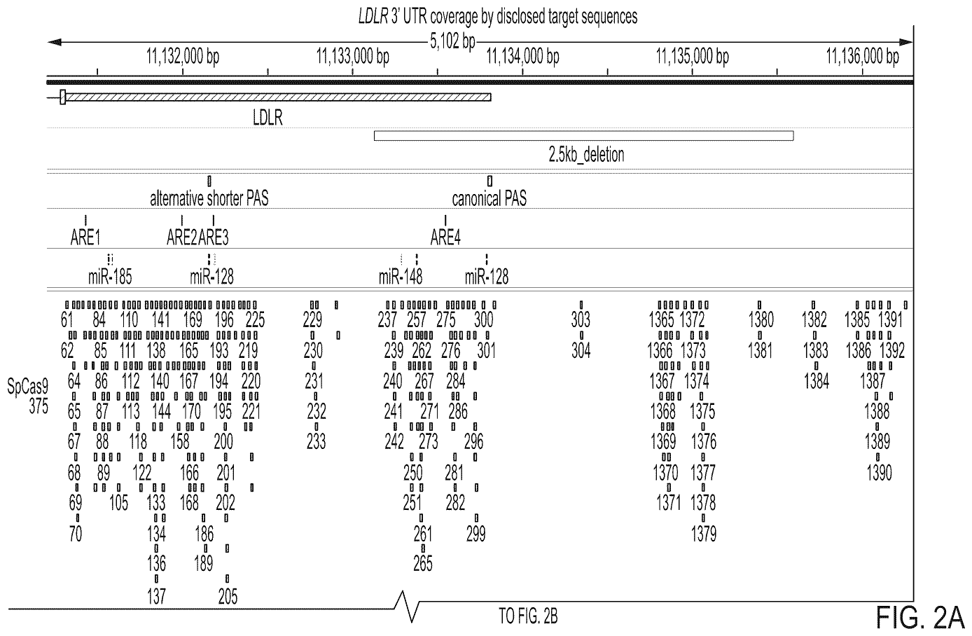

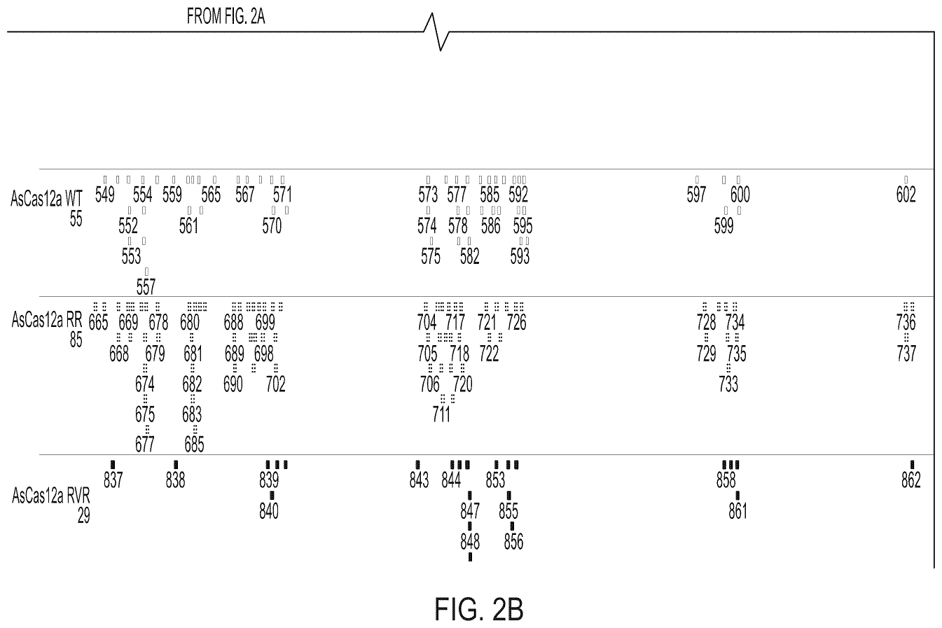

Guide RNAs for use with SpCas9, AsCas12a WT, AsCas12a RR, and AsCas12a RVR were designed to increase coverage (FIG. 2A-2B). The PAM sites for these nucleases are NGG, TTTV, TYCV, and TATV, respectively.

### 8.3 Example 2: Determination of Percent Editing and LDLR Surface Expression with Single- and Dual-Guide AsCas12a Editing in HepG2 Cells

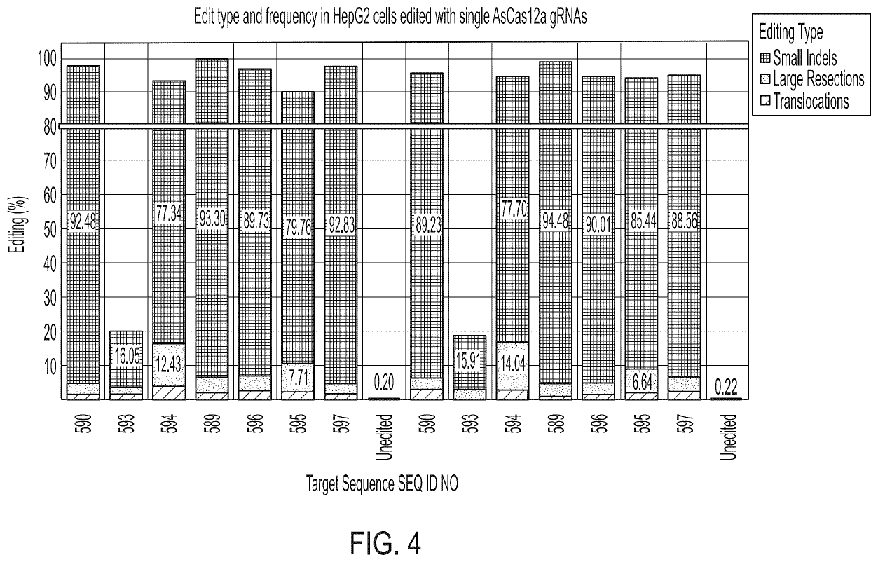

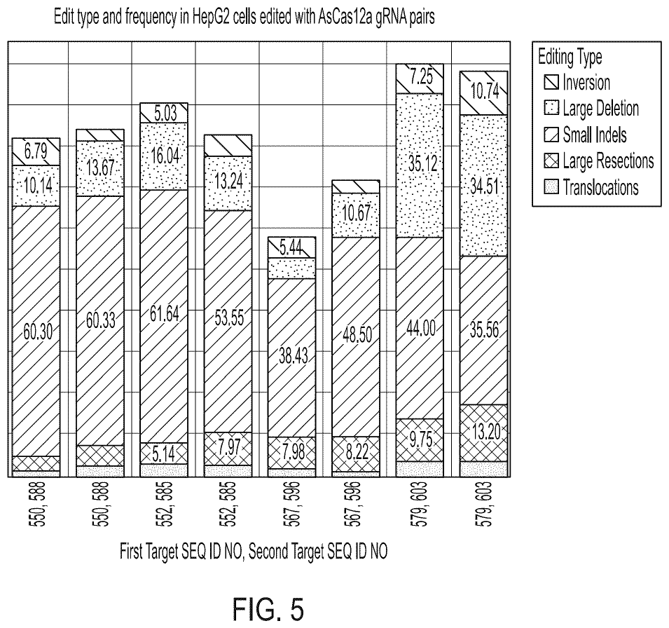

HepG2 cells (ATCC HB-8065) were treated with RNPs containing AsCas12a nuclease (SEQ ID NO: 1348) and single unmodified or dual unmodified AsCas12a gRNAs targeting the 3′ UTR. Edited cells were analyzed using ILL-seq (FIG. 3) and UDiTaS (FIG. 4) to identify the frequency and type of edit(s) induced. Many single gRNAs produced edits in >80% of cells (FIG. 3). The majority of cells edited with an effective single guide exhibited edits that were small insertions or deletions (indels) (75-95%), followed by large deletions (5-35%). When SEQ ID NO: 594, which is located within the canonical polyA site, was targeted, large resections were reproducibly induced in >10% of cells (FIG. 4). The majority of cells edited with dual gRNAs exhibited indels (35-65%), followed by large deletions (2-35%) (FIG. 5).

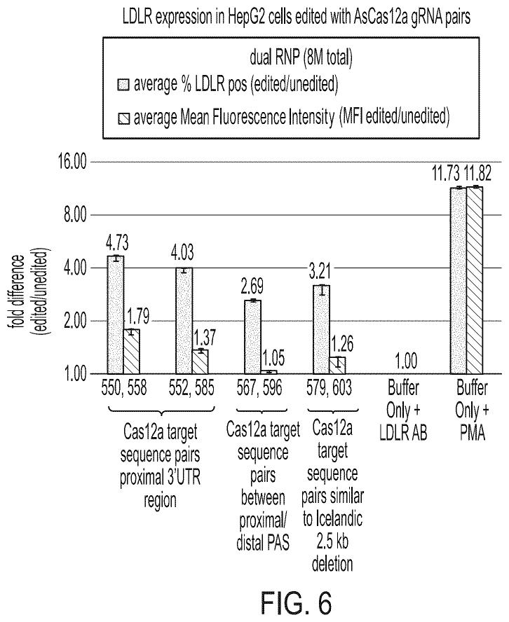

Edited cells were subsequently examined by flow cytometry to detect surface expression levels of LDLR. Dual-guide editing resulted in increased LDLR expression by 2.5-5.0 fold, relative to unedited HepG2 cells (FIG. 6).

### 8.4 Example 3: Determination of Potency and Test Doses with Dual-Guide SpCas9 Editing in HepG2 Cells

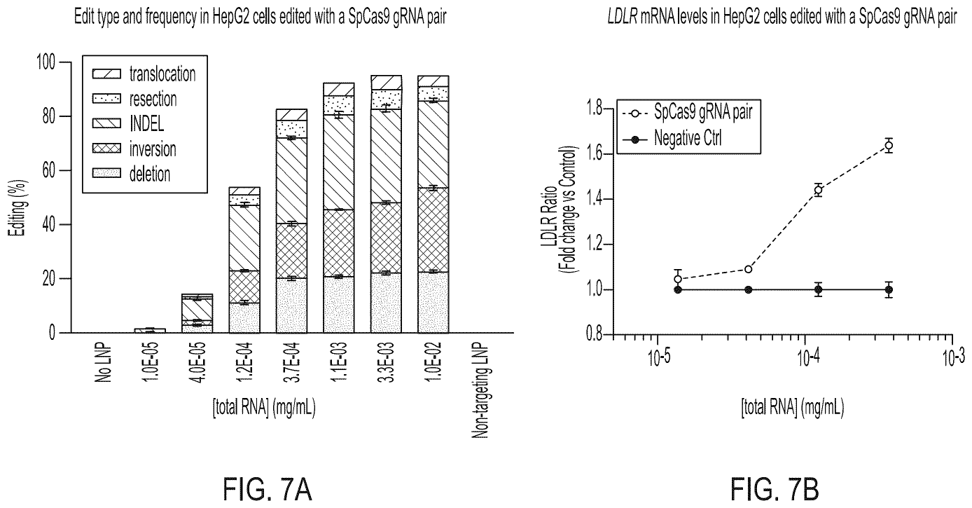

HepG2 cells were treated with LNPs containing mRNA encoding SpCas9 (SEQ ID NO: 1480, CleanCap Cas9 mRNA from TriLink Biotechnologies, which encodes two c-myc NLSs fused to the C-terminal end of the Cas9 molecule) and an exemplary SpCas9 gRNA pair (targeting domain sequences SEQ ID NOs: 79 and 299) targeting the LDLR proximal 3′ UTR at varying total RNA concentrations. HepG2 cells were treated with LNPs containing the indicated total RNA concentrations (i.e., the sum of SpCas9 mRNA and both gRNAs) at a 1:1:1 ratio of SpCas9 mRNA: first gRNA: second gRNA for 4 days (FIGS. 7A-B and 8A-B). Replicates were used for editing analysis via UDiTaS, transcriptional analysis via RT-ddPCR, ELISA for total LDLR protein expression, flow cytometry for LDLR surface protein expression or LDL-C uptake assay. Edited cells were analyzed using UDiTaS sequencing to identify the frequency and type of edit(s) induced at the LDLR 3′ UTR. The percentage of analyzed HepG2 cells with edits exceeded 90% at doses ≥1.1E-3 mg/mL total RNA (FIG. 7A). The largest fraction of these edits was indels, at about 30-40% of treated cells.

Reverse transcription droplet digital polymerase chain reaction (RT-ddPCR) was used to determine LDLR mRNA transcript levels in edited HepG2 cells. LDLR transcript levels increased by >1.4-fold in HepG2 cells edited with ≥10-4 mg/mL total RNA, compared with cells exposed to LNPs containing a non-targeting gRNA (FIG. 7B).

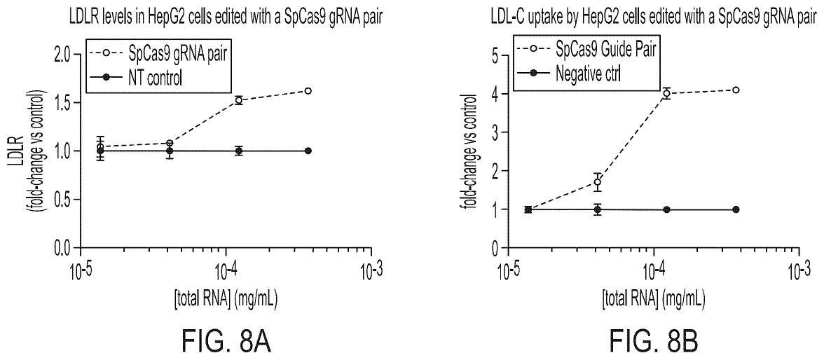

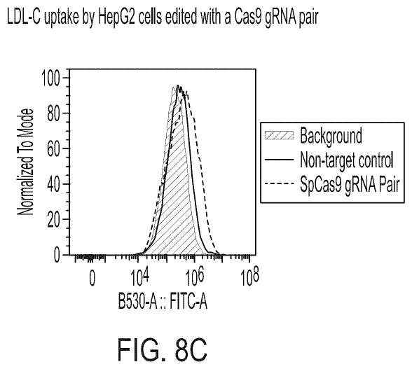

An LDLR Enzyme-Linked ImmunoSorbent Assay (ELISA) was used to determine total LDLR protein levels in edited HepG2 cells. Total LDLR protein levels increased by >1.5-fold in HepG2 cells edited with ≥10-4 mg/mL total RNA, compared with cells exposed to LNPs containing a non-targeting gRNA (FIG. 8A).

A flow cytometry-based cholesterol uptake assay was used to evaluate LDLR function in edited HepG2 cells. This assay involves treating samples with a fluorophore-labeled (DyLight488) LDL-C kit from Abcam (ab236208) and measuring the uptake by flow cytometry. The amount of LDL-C taken up by edited HepG2 cells increased compared with cells exposed to LNPs containing a non-targeting gRNA. FIG. 8B demonstrates the fold change in LDL-C uptake at multiple total RNA concentrations. Upon treatment with LNPs encapsulating >10-4 mg/mL total RNA, LDL-C uptake was increased by greater than about 4-fold compared with cells treated with LNPs containing a non-targeting gRNA. FIG. 8C demonstrates the LDL-C fluorescence in cells treated with LNPs containing 4E-4 mg/mL total RNA.

### 8.5 Example 4: Determination of Potency and Test Doses with Dual-Guide SpCas9 Editing in Primary Human Hepatocytes (PHHs)

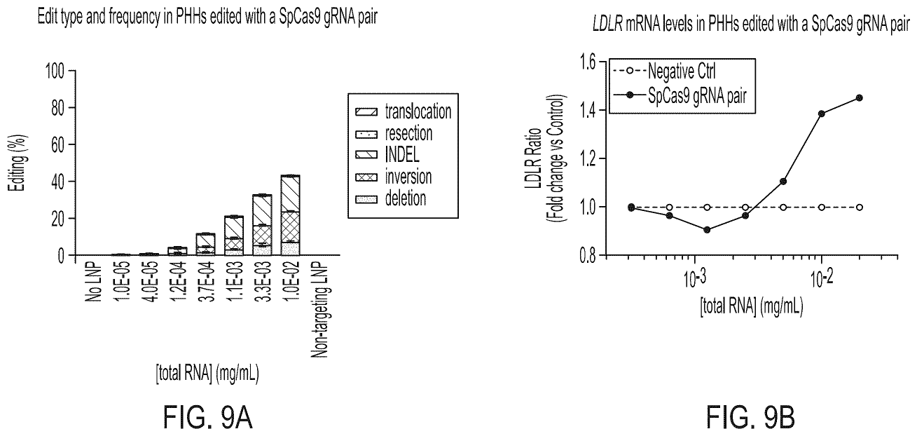

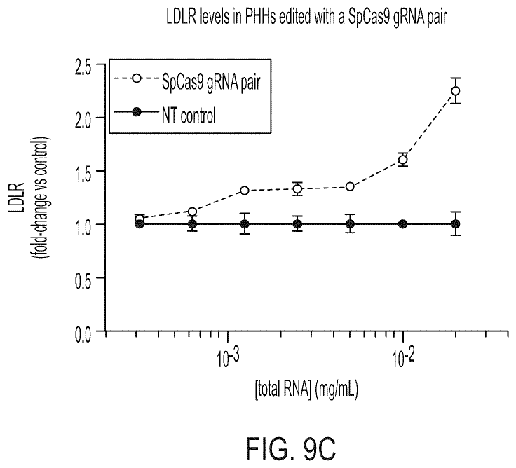

PHHs were treated with LNPs encapsulating mRNA encoding SpCas9 (SEQ ID NO: 1480) and an exemplary SpCas9 gRNA pair (SEQ ID NOs: 918 and 1138) targeting the LDLR proximal 3′ UTR at varying concentrations of total RNA delivered in an LNP format. PHHs were treated with LNPs containing the indicated total RNA concentrations (i.e., the sum of SpCas9 mRNA and both gRNAs) at a 1:1:1 ratio of SpCas9 mRNA: first gRNA: second gRNA for 4 days (FIGS. 9A-C). Replicates were used for editing analysis via UDiTaS, transcriptional analysis via RT-ddPCR, or ELISA for total LDLR protein expression. Edited cells were analyzed using UDiTaS sequencing to identify the frequency and type of edit(s) induced at the LDLR 3′ UTR. At a concentration of 1.1E-3 mg/mL total RNA, ~20% of PHHs exhibited edits of the LDLR 3′ UTR (FIG. 9A). The largest fraction of these edits was indels; ~10% of the total analyzed cell population exhibited this type of edit.

Reverse transcription droplet digital polymerase chain reaction (RT-ddPCR) was used to determine LDLR mRNA transcript levels in edited PHHs. LDLR transcript levels increased by >1.4-fold in PHHs edited with ≥10-2 mg/mL total RNA, compared with cells exposed to LNPs containing a non-targeting gRNA (FIG. 9B).

An LDLR Enzyme-Linked ImmunoSorbent Assay (ELISA) was used to determine total LDLR protein levels in edited PHHs. Total LDLR protein levels increased by >2.0-fold in PHHs edited with 0.02 mg/mL total RNA, compared with cells exposed to a non-targeting LNP (FIG. 9C).

### 8.6 Example 5: Determination of Potency and Test Doses with Dual-Guide AsCas12a (WT) Editing in HepG2 Cells and Primary Human Hepatocytes (PHHs)

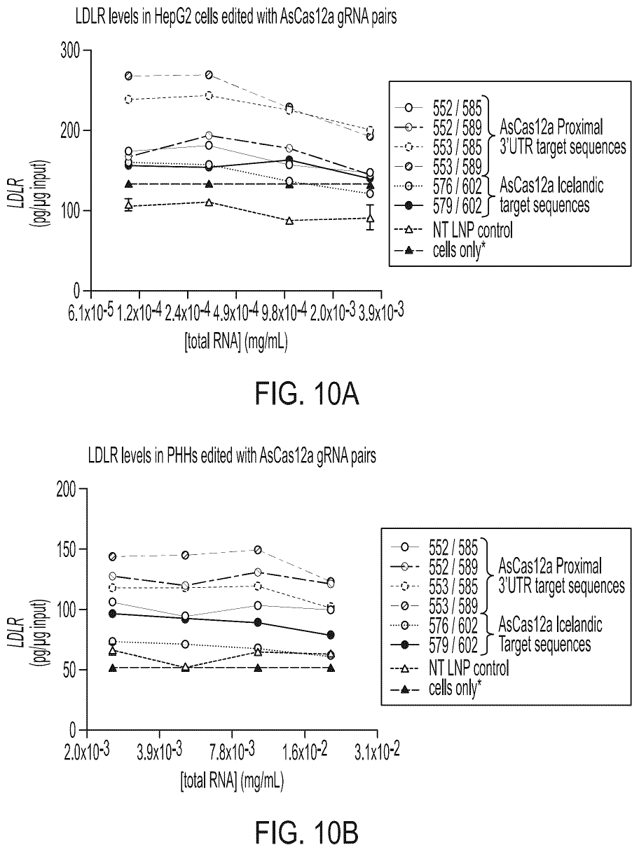

HepG2 cells and PHHs were treated with a plurality of AsCas12a gRNA pairs encapsulated in LNPs with mRNA encoding AsCas12a (SEQ ID NO: 899) at varying concentrations of total RNA (i.e., the sum of both AsCas12a gRNAs) at a 1:1:1 ratio of AsCas12 mRNA: first gRNA: second gRNA for 4 days. The gRNAs comprised the following modifications: 25-mer DNA extension (SEQ ID NO: 7), 5′ and 3′ terminal inverted dTs, and the “conservative 2′F in hairpin” pattern (SEQ ID NO: 35). Cells were treated with LNPs for 4 days. Edited cells were analyzed using UDiTaS sequencing to identify the frequency and type of edit(s) induced at the LDLR 3′ UTR. Treatment with dual gRNAs resulted in total LDLR protein levels increasing by up to 2-fold in HepG2 cells and up to 3-fold in PHHs (FIGS. 10A-10B).

### 8.7 Example 6: Determination of SpCas9 Dual Guide Efficacy in PHHs

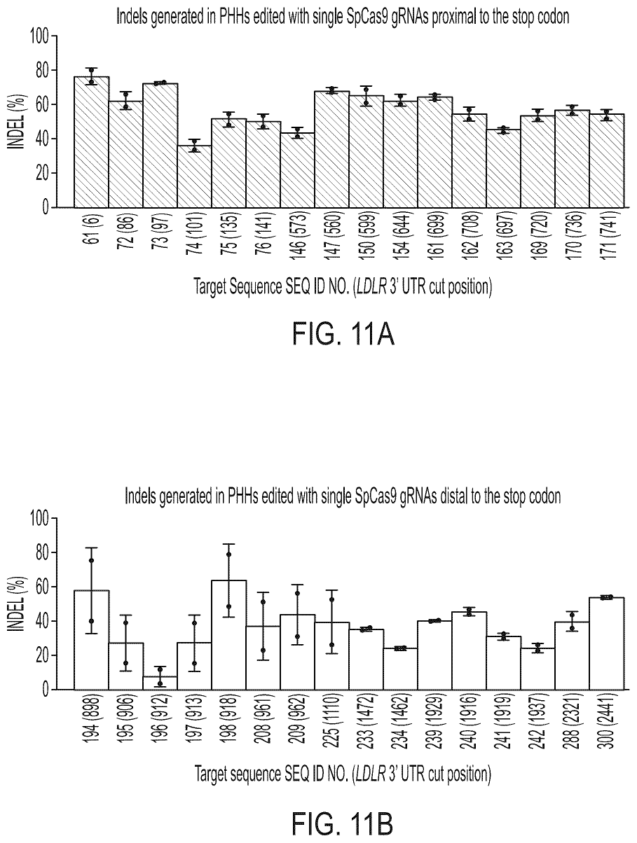

PHHs were cotransfected with single gRNAs for SpCas9 (SEQ ID NOs: 900-1143) and mRNA encoding SpCas9 (SEQ ID NO: 1480) via lipofection with MessengerMax lipofectamine according to the manufacturer's instructions (Thermo Fisher Scientific™). Edited cells were analyzed using ILL-seq to identify the frequency of indels induced at the LDLR 3′ UTR. Among gRNAs located proximal to the stop codon (i.e., within 800 nucleotides downstream), indels were generated in about 30-80% of cells (FIG. 11A). Among gRNAs located distal to the stop codon (i.e., ≥800 nucleotides downstream), indels were generated in about 5-70% of cells (FIG. 11B).

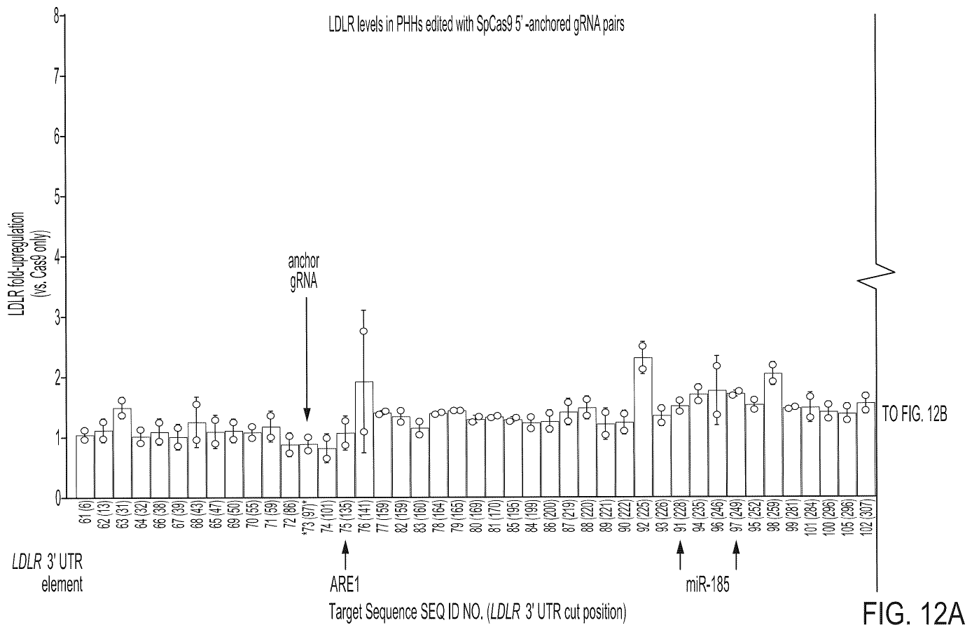

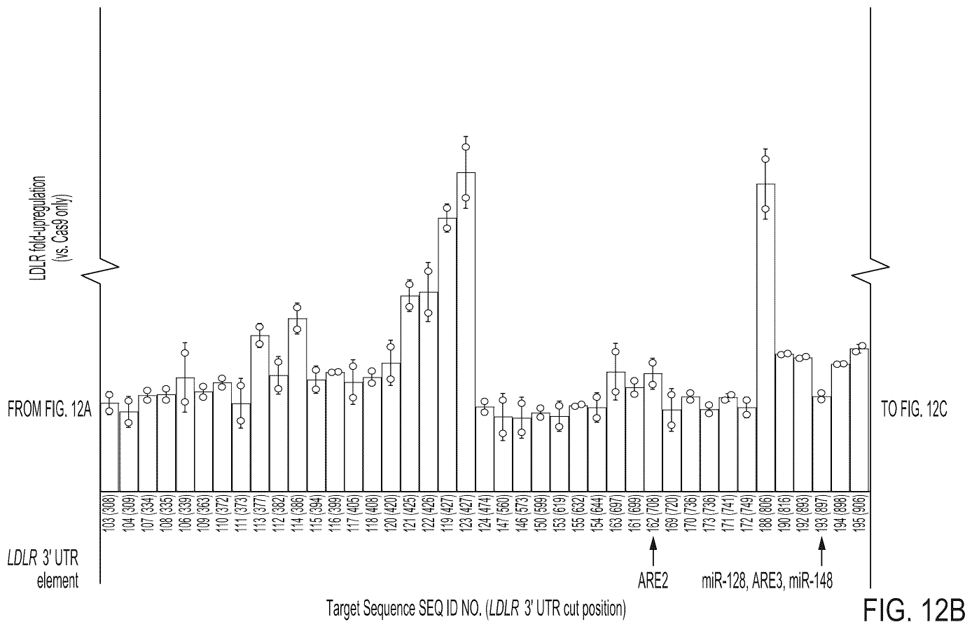

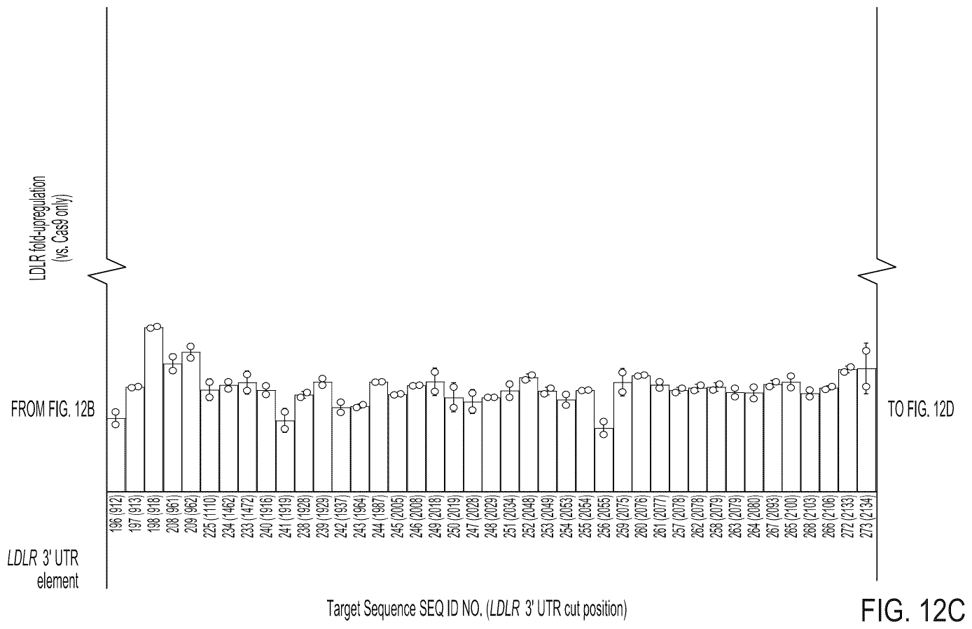

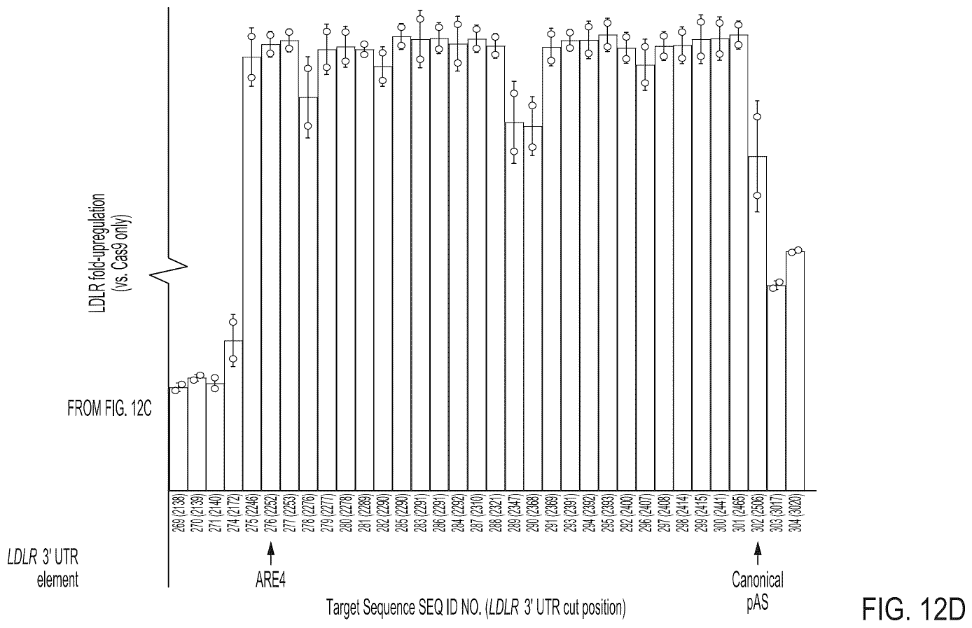

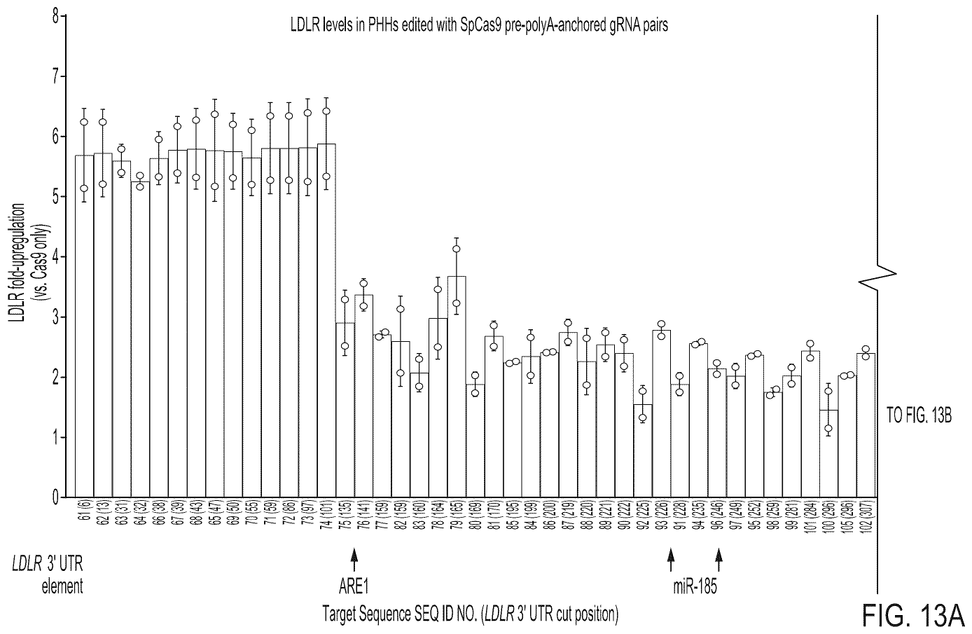

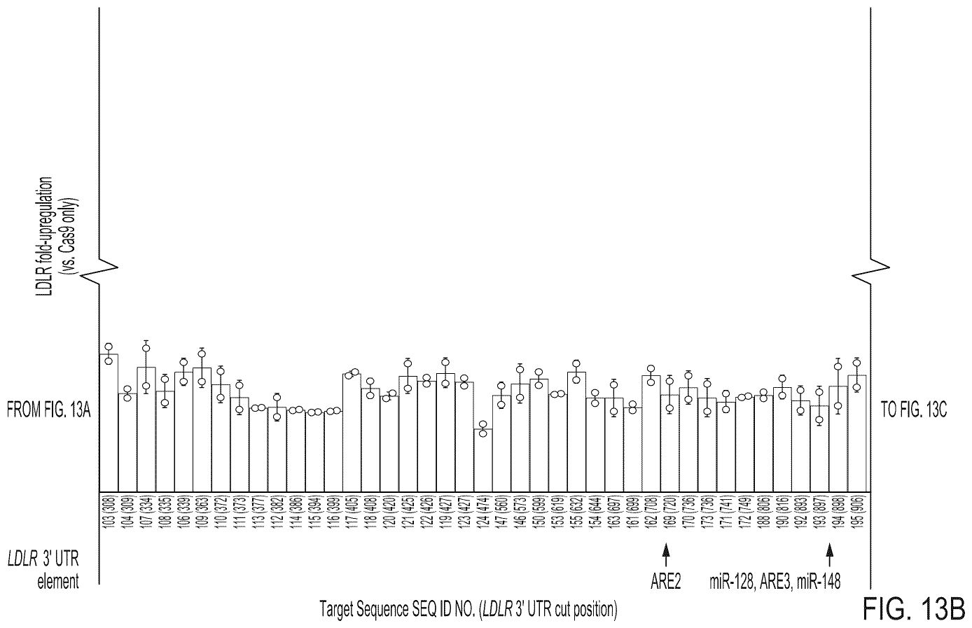

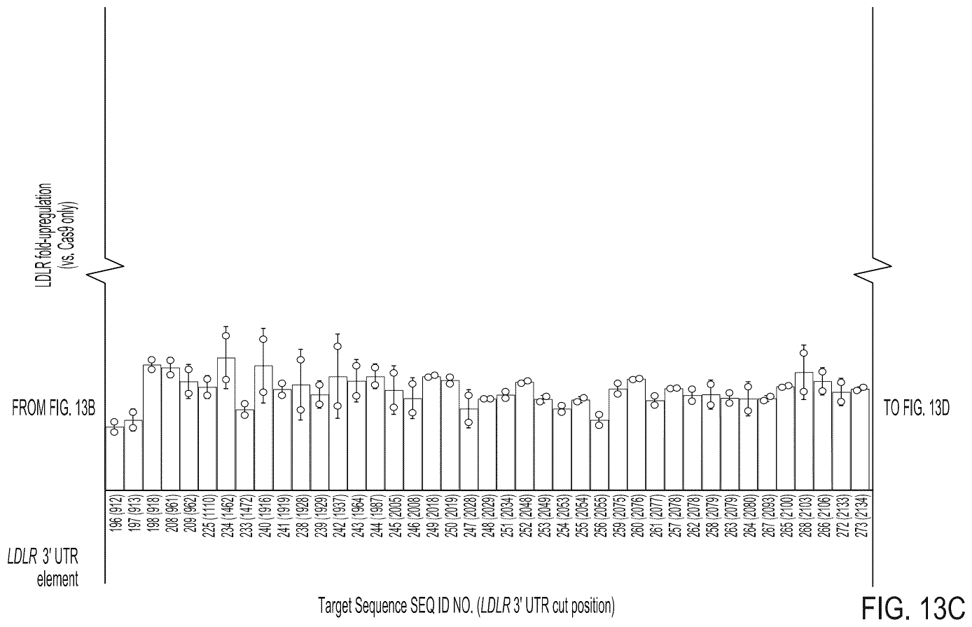

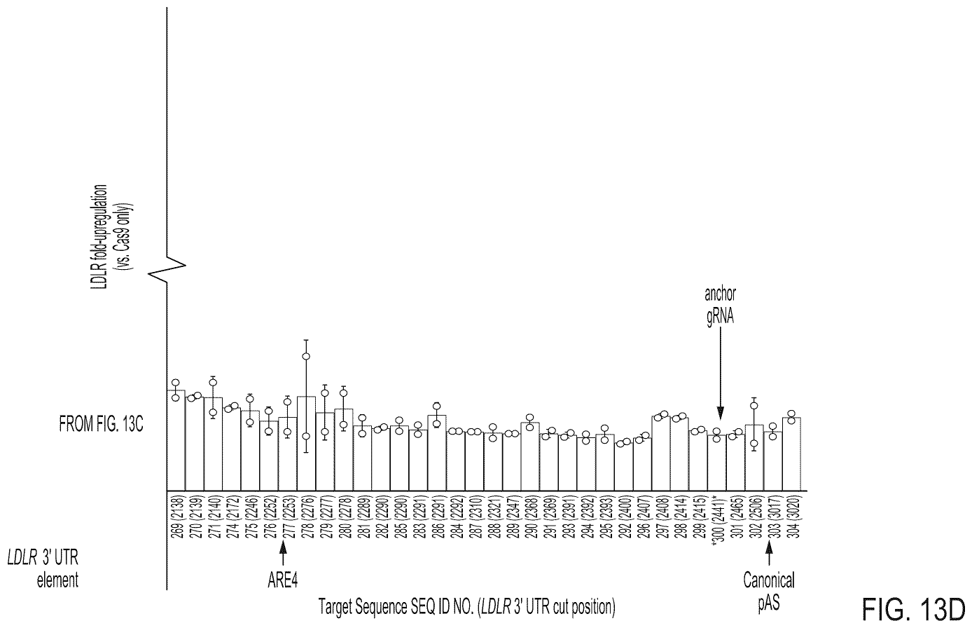

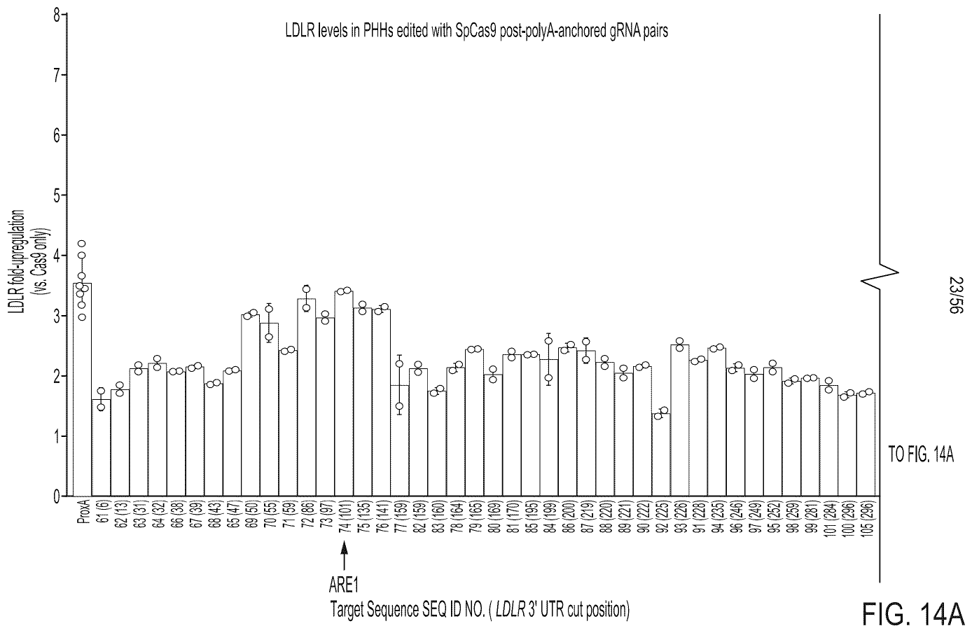

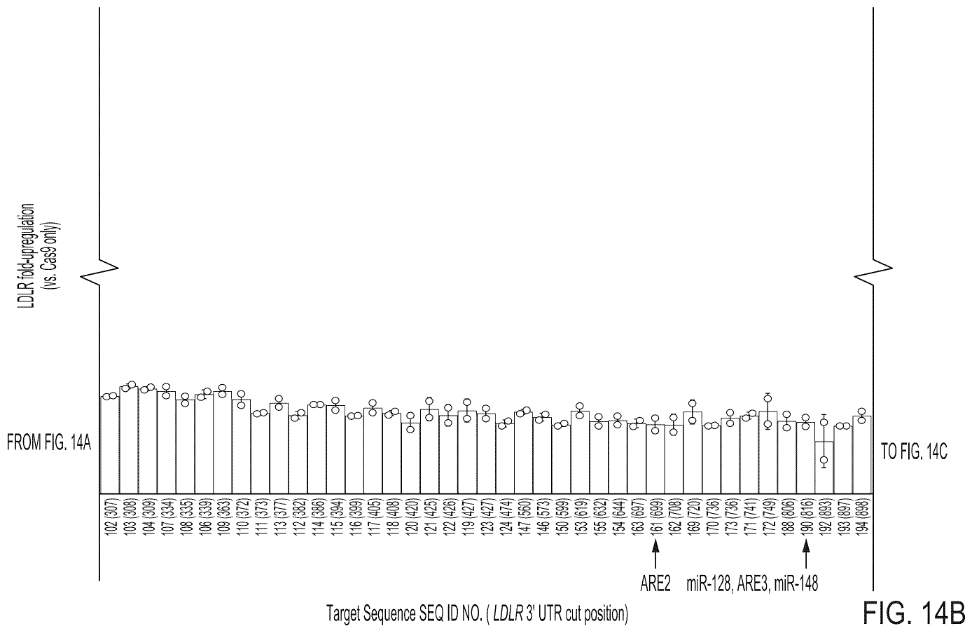

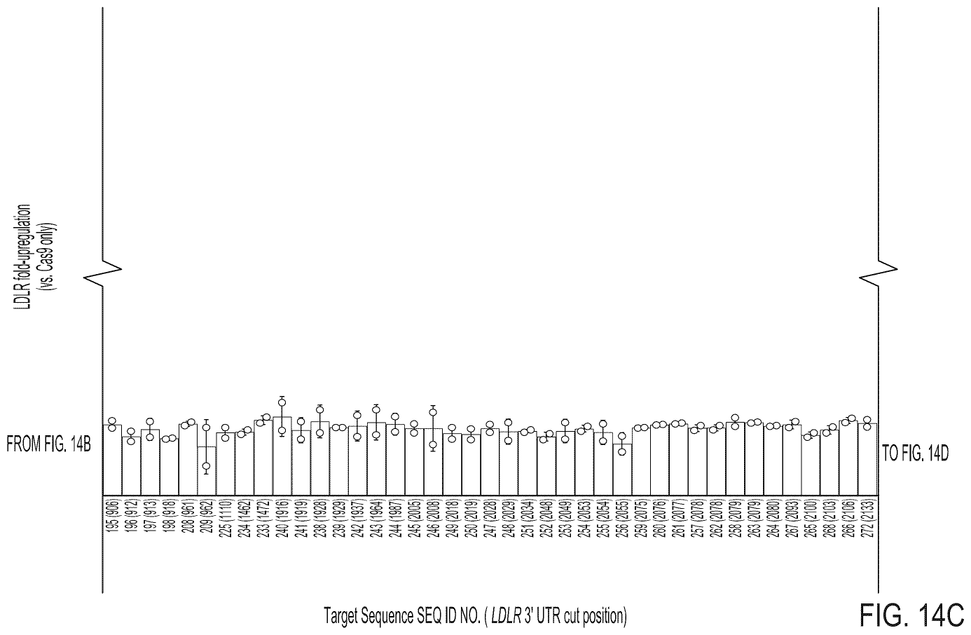

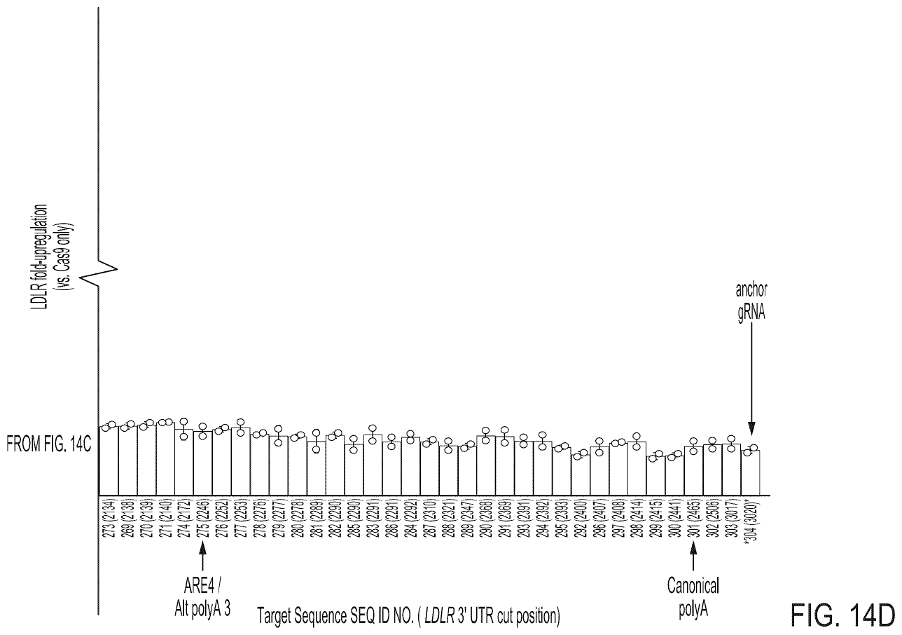

Three different target sequence anchor points were used for anchored gRNA walks to assess the efficacy of different 3′ UTR truncations on LDLR expression: SEQ ID NO: 73 (97 nucleotides downstream from the stop codon); SEQ ID NO: 300 (2441 nucleotides downstream from the stop codon and upstream of the polyA sites); and SEQ ID NO: 304 (3020 nucleotides downstream from the stop codon and downstream of the polyA sites). PHHs were treated with one of the anchor gRNAs and a plurality of second gRNAs, co-delivered with an mRNA containing an SpCas9 ORF (SEQ ID NO: 1349) via lipofection with MessengerMax lipofectamine according to the manufacturer's instructions (Thermo Fisher Scientific). The total LDLR protein level was assessed by ELISA. For all three anchor gRNAs, guide pairs with greater distances between the target sites generally resulted in a comparatively greater increase in total LDLR expression (FIGS. 12A-14D).

Additional gRNA pairs were selected for further testing. Among these additional pairs, greater distances between the pair also generally resulted in a comparatively greater increase in LDLR expression as assessed by ELISA (FIG. 15). As shown in FIG. 15, a significant difference in upregulation effect was identified between the use of the 5′ target sequences of SEQ ID NO: 74 and SEQ ID NO: 75 (both in combination with the 3′ target sequence of SEQ ID NO: 288). Specifically, a 9-fold upregulation was surprisingly observed when the target sequences of SEQ ID NO: 74 and 288 were used, while only a 3-fold upregulation was observed when using the target sequences of SEQ ID NO: 75 and 288 (a significant, 6-fold difference in upregulation). The defining difference between these two deletions is whether or not the ARE1 element is deleted, along with all other miRNA binding sites and ARE elements. Thus, the critical ARE1-associated sequence (ATTTA) is between where these two gRNAs cut (GAGCTTTGTTTTATATATTTATTCATCTGGGAGGCAGAAC; SEQ ID NO: 1482). It is also important to note that small deletions of this region, alone, surprisingly did not induce significant upregulation, indicating that the ARE1 element alone is not sufficient to de-repress, but is likely redundant in function with other ARE elements and, therefore, all must be deleted for maximal effect. Thus, the selection of the 5′ and 3′ target sequences and designing and selection of the associated gRNA pairs is critical to achieving the significant upregulation of LDLR.

### 8.8 Example 7: Determination of AsCas12a (WT) Dual Guide Efficacy in PHHs

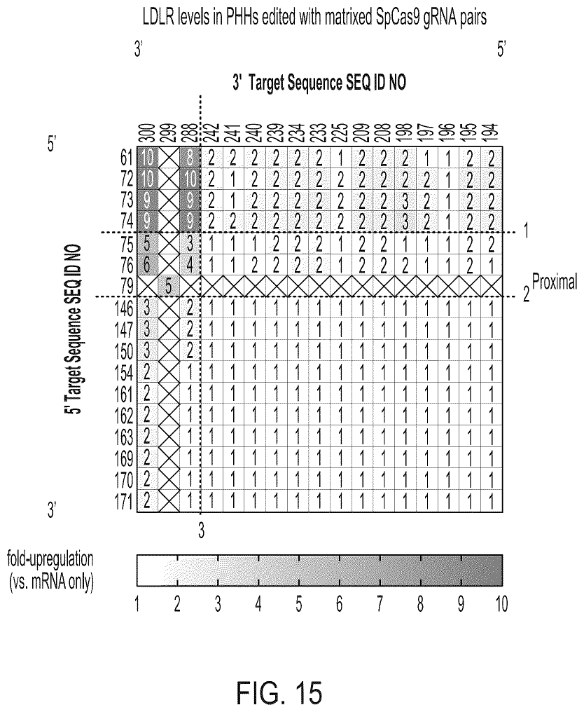

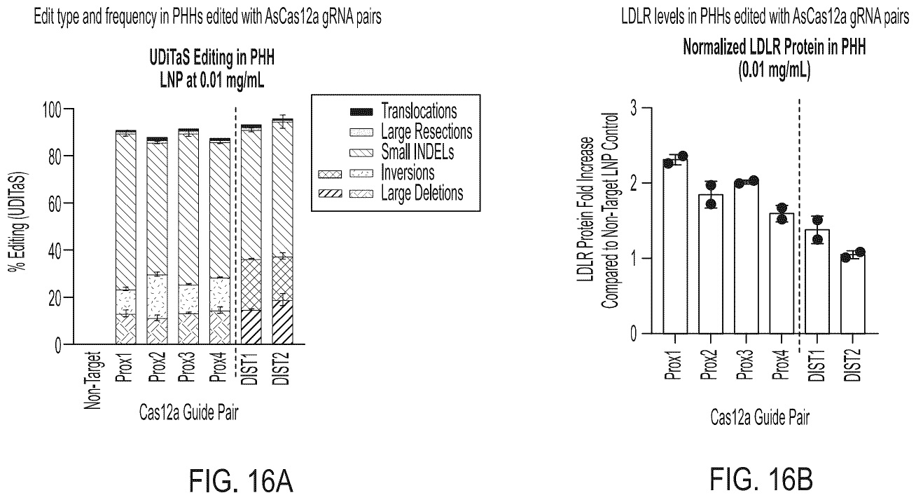

Six AsCas12a (WT) gRNA pairs were selected with target sequences in either the proximal 3′ UTR (4 pairs) or the more distal region impacted by the 2.5 kb deletion present in the Icelandic mutation (2 pairs) (Table 24). PHHs were treated for 4 days with 10-2 mg/mL RNA-LNPs containing mRNA encoding AsCas12a (SEQ ID NO: 899) and gRNAs with the following modifications: 25-mer DNA extension (SEQ ID NO: 7), 5′ and 3′ terminal inverted dTs, and the “conservative 2′F in hairpin” pattern (SEQ ID NO: 35). The frequency and type of edit was assessed by UDiTaS and total LDLR protein level was assessed by ELISA. The frequency of total edits was about 80-95% of PHHs tested. Inversions or large deletions were observed in about 20-40% of edited cells for all 6 gRNA pairs (FIG. 16A). Guide RNA pairs targeted to the proximal 3′ UTR resulted in an about 1.5-2.5-fold increase in total LDLR expression relative to non-editing “Control” or “Vehicle” conditions. Guide RNA pairs targeted to the region of the Icelandic mutation resulted in up to a 1.5-fold increase in LDLR expression (FIG. 16B).

TABLE 24
Target Sequence Pairs for LDLR in PHHs

| | 5′ | 3′ | |
|---|---|---|---|
| Nickname | SEQ ID NO | SEQ ID NO | Deletion |
| Prox4 | 552 | 585 | Prox 3′UTR |
| Prox3 | 552 | 589 | Prox 3′UTR |
| Prox2 | 553 | 585 | Prox 3′UTR |
| Prox1 | 553 | 589 | Prox 3′UTR |
| DIST2 | 576 | 602 | Dist 3′ UTR |
| DIST1 | 579 | 602 | Dist 3′ UTR |

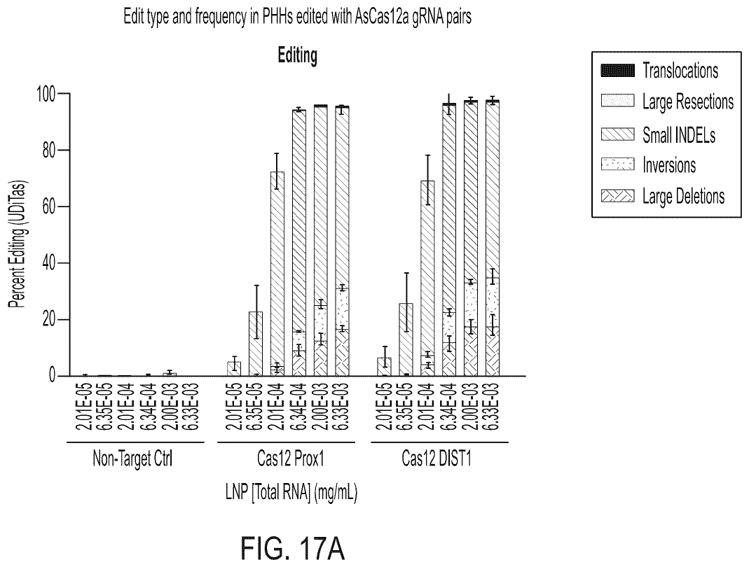

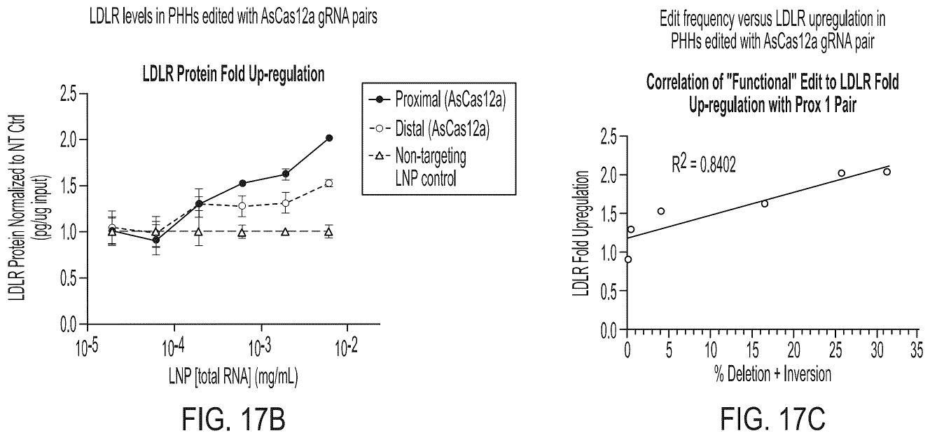

A single pair each targeting the proximal 3′ UTR and the region of the Icelandic mutation were selected for dose-response testing. Increasing total RNA concentrations resulted in increased generation of inversions and large deletions in PHH populations as assessed by UDiTaS (FIG. 17A). Increasing total RNA concentrations resulted in larger fold increases in LDLR total protein expression as assessed by ELISA (FIG. 17B). The increased generation of inversions and large deletions correlated with the fold-change in LDLR total protein expression with R2 =0.8402 (FIG. 17C).

### 8.9 Example 8: Determination of SpCas9 and AsCas12a Dual Guide Induction of Edits and Impact on Total LDLR Expression in Primary Human Hepatocytes (PHHs)

One AsCas12a (WT) and one SpCas9 gRNA pair were selected with target sequences to induce the Proximal deletion (Table 25A), and targeting domain sequences are presented in Table 25B. For the AsCas12a gRNA pair, PHHs were treated for 4 days with LNPs containing mRNA encoding AsCas12a (SEQ ID NO: 899) and gRNAs with the following modifications: 25-mer DNA extension (SEQ ID NO: 7), 5′ and 3′ terminal inverted dTs, and the “conservative 2′F in hairpin” pattern (SEQ ID NO: 35) (Table 25C). For the SpCas9 gRNA pair (Table 25C) PHHs were treated for 4 days with LNPs containing mRNA encoding SpCas9 (SEQ ID NO: 1480, CleanCap Cas9 mRNA from TriLink Biotechnologies) and the gRNA pair.

TABLE 25A
Target Sequences for Proximal deletion by SpCas9 or AsCas12a gRNA
Pairs

| | Relative | | |
|---|---|---|---|
| | Guide | | |
| Pair Nickname | Position | SEQ ID NO | Target Sequence (DNA) |
| SpCas9 | 5′ | 79 | CAGTGCCCATGCAATGGCTT |
| Proximal | 3′ | 299 | ATGAGTGGCTTCTGGGAGAT |
| AsCas12a | 5′ | 553 | TCTTATCCTTCACGAGGAAAG |
| Proximal | 3′ | 589 | GGAGAATGATGTCCCCGTTGT |

TABLE 25B
Targeting Domain Sequences for Proximal deletion by SpCas9 or AsCas12a
gRNA Pairs

| | Relative | | |
|---|---|---|---|
| Pair | Guide | SEQ | |
| Nickname | Position | ID NO | Targeting Domain Sequence (RNA) |
| SpCas9 | 5′ | 1468 | mC*mA*mG*rTrGrCrCrCrArTrGrCrArArTrGrGrCrTrT |
| Proximal | 3′ | 1470 | mA*mT*mG*rArGrTrGrGrCrTrTrCrTrGrGrGrArGrArT |
| AsCas12a | 5′ | 611 | rUrCrUrUrArUrCrCrUrUrCrArCrGrArGrGrArArArG |
| Proximal | 3′ | 589 | rGrGrArGrArArUrGrArUrGrUrCrCrCrCrGrUrUrGrU |

TABLE 25C
gRNA Sequences for Proximal deletion by SpCas9 or AsCas12a gRNA Pairs

| | Relative | | |
|---|---|---|---|
| Pair | Guide | SEQ | |
| Nickname | Position | ID NO | gRNA Sequence (RNA) |
| SpCas9 | 5′ | 1469 | mC*mA*mG*rTrGrCrCrCrArTrGrCrArArTrGrGrCrTr |
| Proximal | | | TrGrUrUrUrUrArGrAmGmCmUmAmGmAmAmAmU |
| | | | mAmGmCrArArGrUrUrArArArArUrArArGrGrCrUrAr |
| | | | GrUrCrCrGrUrUrArUrCrAmAmCmUmUmGmAmAm |
| | | | AmAmAmGmUmGmGmCmAmCmCmGmAmGmUm |
| | | | CmGmGmUmGmCmU*mU*mU*mU |
| | 3′ | 1471 | mA*mT*mG*rArGrTrGrGrCrTrTrCrTrGrGrGrArGrAr |
| | | | TrGrUrUrUrUrArGrAmGmCmUmAmGmAmAmAmU |
| | | | mAmGmCrArArGrUrUrArArArArUrArArGrGrCrUrAr |
| | | | GrUrCrCrGrUrUrArUrCrAmAmCmUmUmGmAmAm |
| | | | AmAmAmGmUmGmGmCmAmCmCmGmAmGmUm |
| | | | CmGmGmUmGmCmU*mU*mU*mU |
| AsCas12a | 5′ | 1148 | rUrArArUrUrUrCrUrArCrUrCrUrUrGrUrArGrArUrUr |
| Proximal | | | CrUrUrArUrCrCrUrUrCrArCrGrArGrGrArArArG |
| | 3′ | 1184 | rUrArArUrUrUrCrUrArCrUrCrUrUrGrUrArGrArUrGr |
| | | | GrArGrArArUrGrArUrGrUrCrCrCrCrGrUrUrGrU |

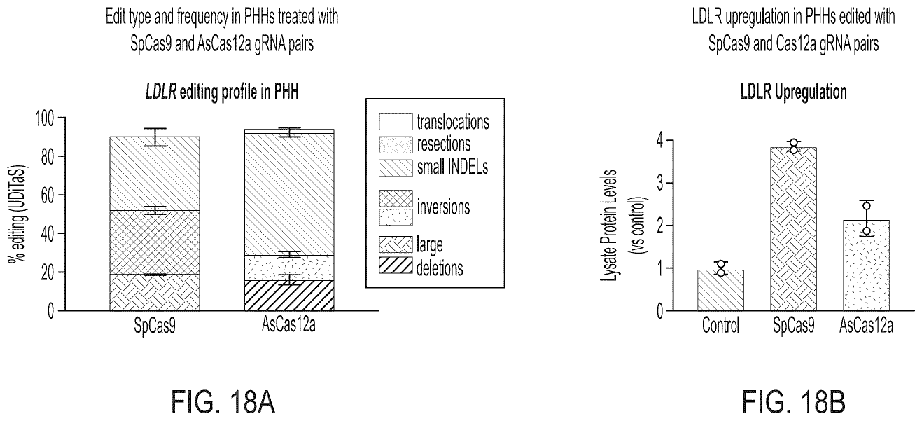

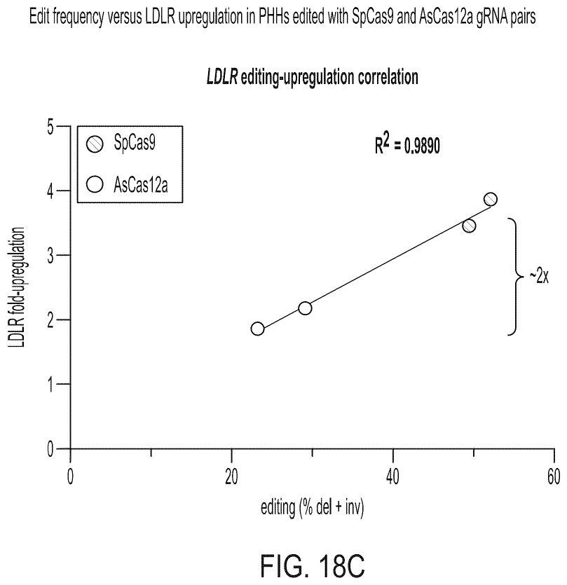

The frequency and type of edit was assessed by UDiTaS and total LDLR protein level was assessed by ELISA. The total frequency of edits was about 85-95% of PHHs tested. Cells edited with the SpCas9 gRNA pair exhibited a larger percentage of functional edits (i.e. the sum of inversions and large deletions) (about 52%) than cells edited with the AsCas12a pair (about 29%) (FIG. 18A). The SpCas9 gRNA pair exhibited higher LDLR upregulation (about 3.8-fold) than the AsCas12a gRNA pair (about 2.2-fold) (FIG. 18B). There was a strong correlation (R2=0.9890) between functional editing and LDLR expression (FIG. 18C).

### 8.10 Example 9: Determination of Potency and Doses with Dual-Guide SpCas9 Editing in Primary Human Hepatocytes (PHHs) and HepG2 Cells

PHHs were treated with a plurality of SpCas9 gRNA pairs encapsulated in LNPs with mRNA encoding SpCas9 (SEQ ID NO: 1480, CleanCap Cas9 mRNA from TriLink Biotechnologies, which includes two c-myc NLSs (PAAKRVKLD) fused to the C-terminal end of Cas9) at a range of doses of total RNA (i.e., the sum of Cas9 mRNA and both SpCas9 gRNAs) at a 1:1:1 ratio of SpCas9 mRNA: first gRNA: second gRNA. Cells were treated with LNPs for 4 days.

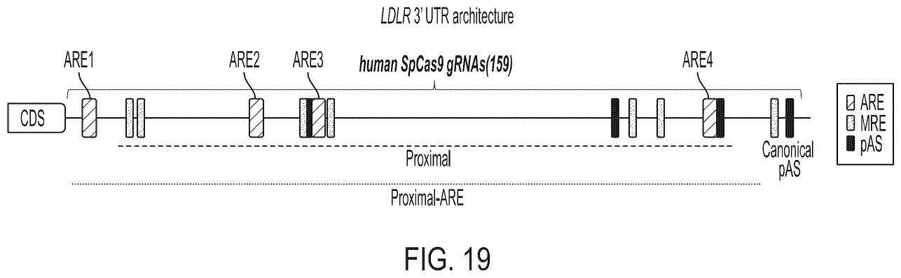

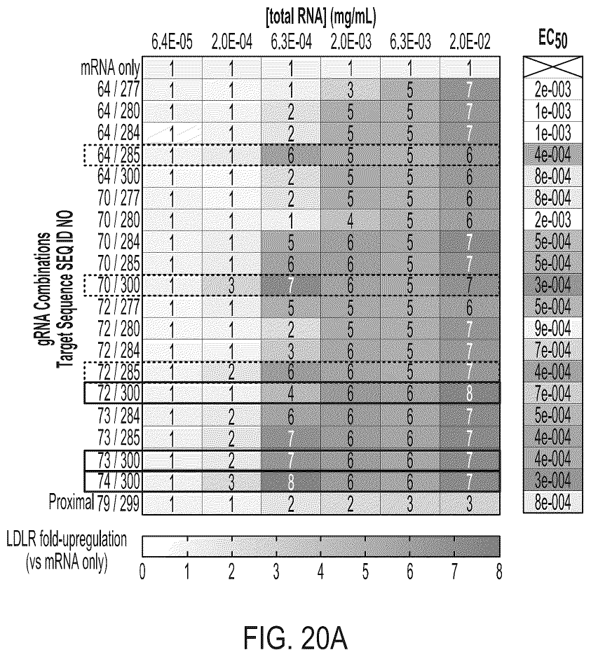

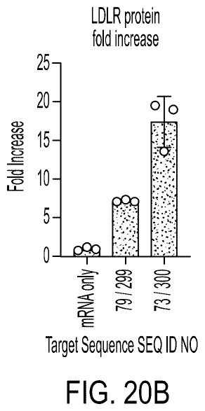

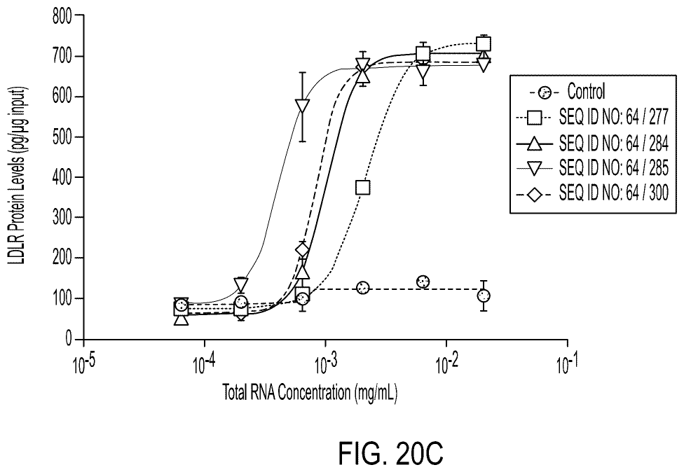

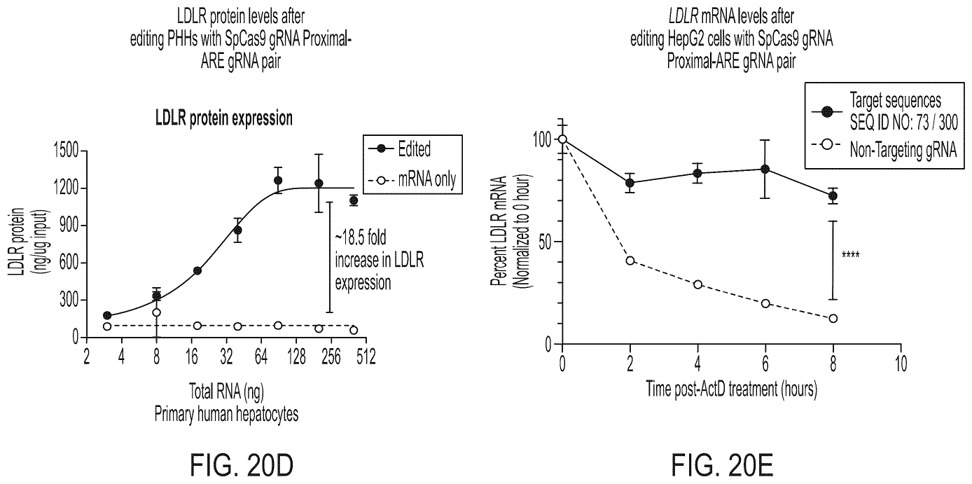

An LDLR Enzyme-Linked ImmunoSorbent Assay (ELISA) was used to determine total LDLR protein levels in edited PHHs. Treatment with dual gRNAs selected to induce the Proximal deletion (Target Sequences SEQ ID NO: 79 and 299) resulted in total LDLR protein levels increasing by up to about 7-fold (FIG. 18B, FIG. 20A and FIG. 20B). Surprisingly, treatment with dual gRNAs that induce the Proximal-ARE deletion (Target Sequences SEQ ID NO: 73 and 300), which differs at least by including the first AU-rich element (ARE1) (FIG. 19) resulted in total LDLR protein levels increasing by up to about 17-fold (FIG. 20A-20C). The EC₅₀ for the LNP total mRNA (i.e., the sum of SpCas9 mRNA and both gRNAs) was 8e-04 mg/mL for dual gRNAs that induce the Proximal deletion and 2-8e-04 mg/mL for dual gRNAs that induce the Proximal-ARE deletion (FIG. 20A and Tables 26-27). Human-specific gRNA pairs (e.g., 5′ Target Sequences SEQ ID NO: 64 and 70 and 3′ Target Sequences SEQ ID NO: 284 and 285) perform comparably to NHP cross-reactive pairs (e.g., 5′ Target Sequences SEQ ID NO: 72 and 73 and 3′ Target Sequence SEQ ID NO: 300).

TABLE 26

| Target | Max Protein | |
|---|---|---|
| Sequence | Upregulation | |
| SEQ ID NO | (vs. control) | EC₅₀ |
| Control | 1 | N/A |
| 64/277 | 6.8 | 2.1 × 10⁻³ |
| 64/284 | 6.6 | 1.0 × 10⁻³ |
| 64/285 | 6.3 | 4.0 × 10⁻⁴ |
| 64/300 | 6.4 | 8.2 × 10⁻⁴ |

PHHs were further treated with dual gRNAs that induce the Proximal-ARE deletion (Target Sequences SEQ ID NO: 73 and 300) via lipofection with MessengerMax lipofectamine according to the manufacturer's instructions (Thermo Fisher Scientific™). Guide RNAs were manufactured by Axolabs™, with modifications as shown in SEQ ID NO: 1477 and 1450 (Table 33C). Total LDLR protein levels as assessed by ELISA increased by up to 18.5-fold compared with cells transfected with SpCas9 mRNA only (FIG. 20D).

HepG2 cells were treated as above with a single gRNA pair selected to induce the Proximal-ARE deletion (Target Sequences SEQ ID NO: 73 and 300), encapsulated in LNPs with mRNA encoding SpCas9 (SEQ ID NO: 1480, CleanCap Cas9 mRNA from TriLink Biotechnologies), or LNPs encapsulating a non-targeting gRNA control, with the exception that the period of treatment with LNPs was 3 days. Cells were then treated with 10 μM Actinomycin D (ActD). At multiple time points after treatment, the normalized LDLR mRNA levels were determined. LDLR-edited HepG2 cells maintained at least about 70% of the baseline level of LDLR mRNA until at least 8 hours after ActD treatment; HepG2 cells treated with a non-targeting control gRNA exhibited decreasing levels of LDLR mRNA at 2, 4, 6, and 8 hours after ActD treatment, with about 10% LDLR mRNA remaining at 8 hours (FIG. 20E).

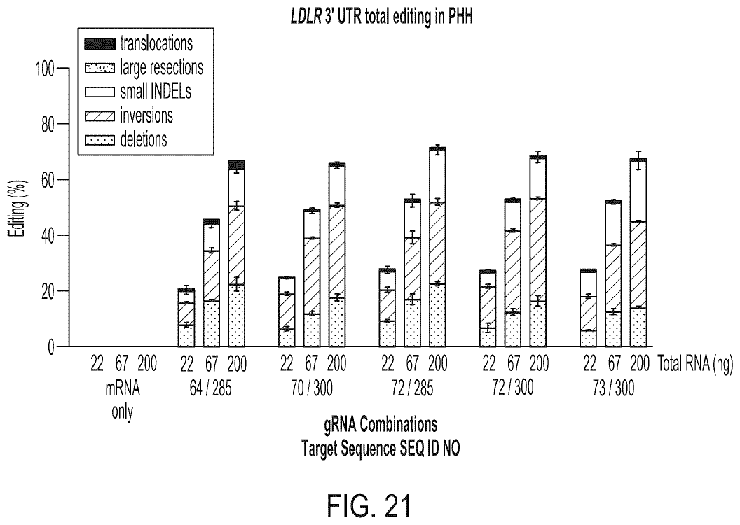

PHHs were further treated with a plurality of SpCas9 gRNA pairs selected to induce the Proximal-ARE deletion. The SpCas9 gRNA pairs were encapsulated in LNPs with mRNA encoding SpCas9 (SEQ ID NO: 1480, CleanCap Cas9 mRNA from TriLink Biotechnologies) at three doses (22, 67, and 200 ng) of total RNA (i.e., the sum of Cas9 mRNA and both SpCas9 gRNAs) at a 1:1:1 ratio of SpCas9 mRNA: first gRNA: second gRNA. Cells were treated with LNPs for 4 days. Edited cells were analyzed using UDiTaS sequencing to identify the frequency and type of edit(s) induced at the LDLR 3′ UTR. The percentage of analyzed PHHs with functional edits (i.e., the sum of inversions and deletions) exceeded about 35% at doses of 200 ng total RNA (FIG. 21).

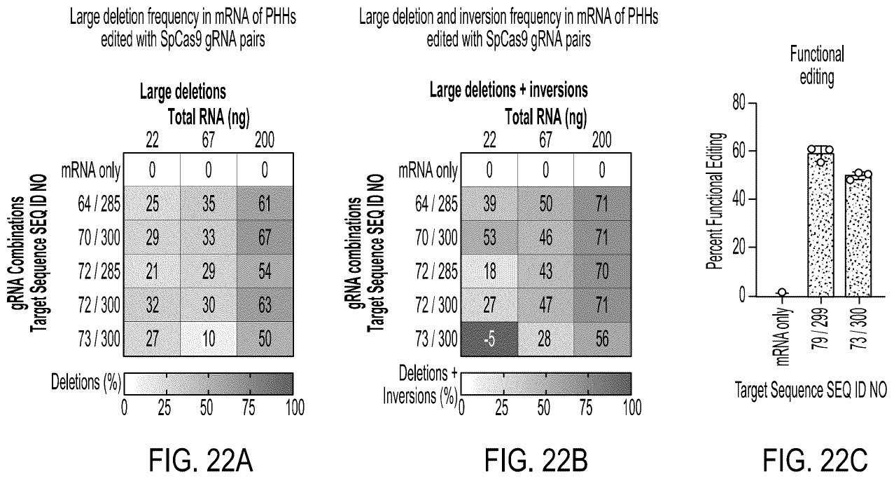

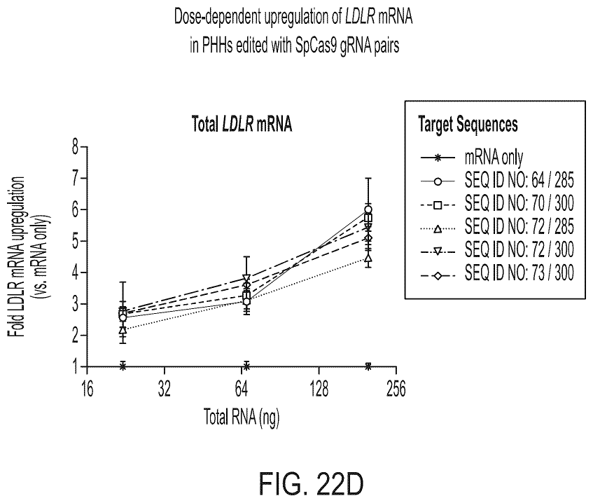

Reverse transcription droplet digital polymerase chain reaction (RT-ddPCR) was used to assess the frequency and type of edit(s) induced at the LDLR 3′ UTR in LDLR mRNAs and to determine LDLR mRNA transcript levels in edited PHHs. Large deletions were detected in about 50-70% of transcripts in PHHs edited with a dose of 200 ng total RNA, compared with cells exposed to LNPs containing nuclease mRNA only (FIG. 22A). Functional edits (i.e., the sum of large deletions and inversions) were detected in about 55-75% of transcripts in PHHs edited with a dose of 200 ng total RNA, compared with cells exposed to LNPs containing nuclease mRNA only (FIG. 22B). Treatment with dual gRNAs that induce the Proximal deletion (Target Sequences SEQ ID NO: 79 and 299) and the Proximal-ARE deletion (Target Sequences SEQ ID NO: 73 and 300), resulted in about 50-60% of cells with functional editing (FIG. 22C). LDLR mRNA levels increased by about 4.5-6-fold in PHHs edited with a dose of 200 ng total RNA, compared with cells exposed to LNPs containing nuclease mRNA only (FIG. 22D).

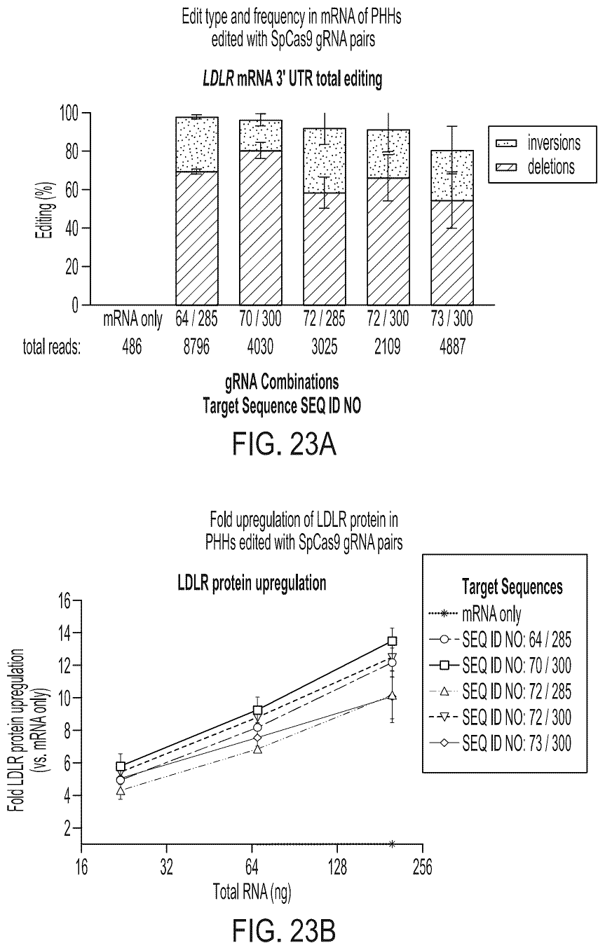

RNA seq was used to assess the frequency and type of edit(s) induced at the LDLR 3′ UTR in LDLR mRNA in edited PHHs. Functional edits (i.e., the sum of large deletions and inversions) were detected in about 80-98% of transcripts in PHHs edited with a dose of 200 ng total RNA, compared with cells exposed to LNPs containing nuclease mRNA only (FIG. 23A). An LDLR Enzyme-Linked ImmunoSorbent Assay (ELISA) was used to determine total LDLR protein levels in edited PHHs. Total LDLR protein levels increased by >4.5-6-fold in PHHs edited with a dose of 200 ng total RNA, compared with cells exposed to LNPs containing nuclease mRNA only (FIG. 23B).

TABLE 27
LNP dose range finding for selected SpCas9 pairs in PHH

| 5′ Target | 3′ Target | | | | | | |
|---|---|---|---|---|---|---|---|
| Sequence | Sequence | | Fit | Max | 6.3 × 10⁻⁴ | 2.0 × 10⁻⁴ | |
| SEQ ID NO | SEQ ID NO | EC₅₀ | (R²) | effect | mg/mL | mg/mL | Notes |
| 64 | 277 | 2.1E−03 | 0.99 | 6.8 | 1.1 | 0.8 | |
| | 280 | 9.5E−04 | 1.00 | 6.5 | 2.0 | 0.9 | |
| | 284 | 1.0E−03 | 0.99 | 6.6 | 1.7 | 0.8 | |
| | 285 | 4.0E−04 | 0.99 | 6.3 | 5.8 | 1.4 | |
| | 300 | 8.2E−04 | 1.00 | 6.4 | 2.2 | 0.7 | |
| 70 | 277 | 8.2E−04 | 1.00 | 6.2 | 1.9 | 0.8 | |
| | 280 | 1.6E−03 | 0.94 | 5.9 | 1.1 | 0.9 | |
| | 284 | 5.1E−04 | 1.00 | 7.3 | 5.4 | 1.2 | |
| | 285 | 4.6E−04 | 0.99 | 6.9 | 5.8 | 1.3 | |
| | 300 | 2.5E−04 | 1.00 | 7.4 | 7.4 | 2.8 | |
| 72 | 277 | 4.7E−04 | 0.97 | 5.8 | 5.1 | 1.3 | |
| | 280 | 9.0E−04 | 1.00 | 6.7 | 2.3 | 0.9 | |
| | 284 | 7.0E−04 | 0.99 | 6.6 | 3.4 | 0.9 | |
| | 285 | 3.5E−04 | 1.00 | 6.5 | 6.3 | 1.9 | |
| | 300 | 6.5E−04 | 1.00 | 7.6 | 4.4 | 1.1 | NHP cross-reactive |
| 73 | 284 | 4.9E−04 | 0.99 | 7.3 | 5.8 | 1.5 | |
| | 285 | 3.8E−04 | 1.00 | 7.4 | 6.9 | 1.8 | |
| | 300 | 4.1E−04 | 1.00 | 7.3 | 6.6 | 1.5 | NHP cross-reactive |
| 74 | 300 | 2.6E−04 | 1.00 | 7.9 | 7.9 | 2.6 | NHP cross-reactive |
| 79 | 299 | 8.3E−04 | 0.93 | 2.9 | 1.8 | 1.0 | |

### 8.11 Example 10: Determination of SpCas9 and AsCas12a Dual Guide Impact on Total LDLR Expression in Primary Human Hepatocytes (PHHs)

PHHs were treated with a plurality of SpCas9 and AsCas12 gRNA pairs selected to induce the Proximal-ARE deletion. For the AsCas12a gRNA pair, PHHs were cotransfected with mRNA encoding AsCas12a (SEQ ID NO: 899) and gRNAs via lipofection with MessengerMax lipofectamine according to the manufacturer's instructions (Thermo Fisher Scientific™). Guide RNAs were manufactured by Integrated DNA Technologies™ For the SpCas9 gRNA pair, PHHs were treated for 4 days with LNPs containing mRNA encoding SpCas9 (SEQ ID NO: 1480, CleanCap Cas9 mRNA from TriLink Biotechnologies) and the gRNA pair. Guide RNAs were manufactured by Integrated DNA Technologies™.

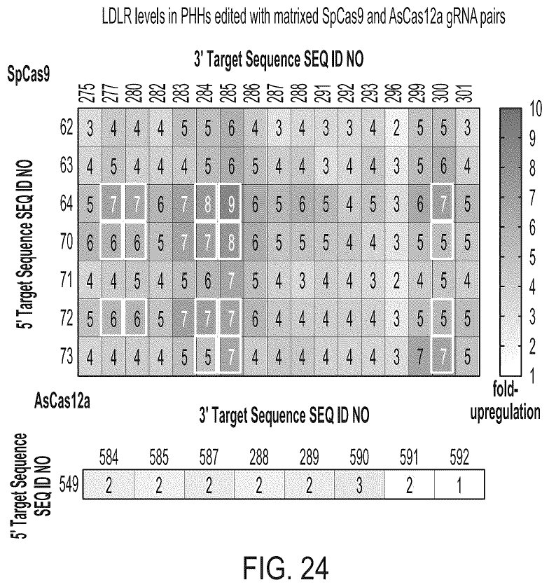

Total LDLR protein level was assessed by ELISA. Editing with SpCas9 gRNA pairs resulted in LDLR upregulation of up to about 9-fold. Editing with AsCas12a resulted in LDLR upregulation of up to about 3-fold. Top-performing SpCas9 gRNA pairs could be resolved at sub-maximal total RNA dose (FIG. 24, Table 28).

TABLE 28
Comparison of deleted regions and Cas effectors in PHH studies

| | | Functional Editing | LDLR protein |
|---|---|---|---|
| Deletion | Cas effector | (deletions + inversions) | upregulation |
| ΔDIST1 | AsCas12a | 35% | 1.5X |
| Proximal | AsCas12a | 29% | 2X |
| | SpCas9 | 52% | 4X |
| Proximal-ARE | AsCas12a | — | 3X |
| | SpCas9 | 55% | 8X |

### 8.12 Example 11: Determination of SpCas9 Proximal-ARE Deletion Impact on Total LDLR Protein Expression and LDL-C Uptake in HepG2 and 1C10 Cells

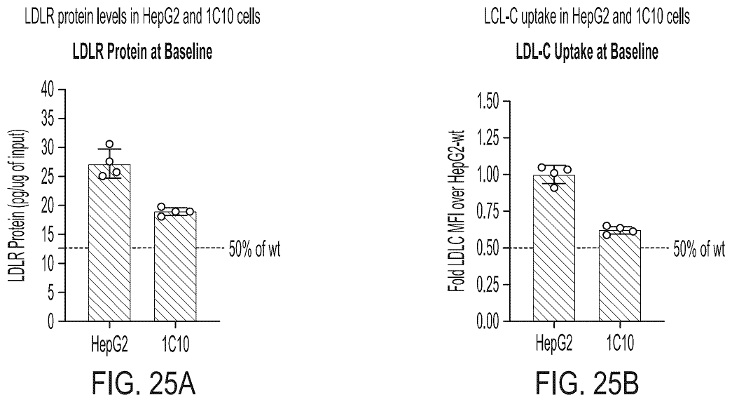

The 1C10 cell line was generated by introducing a heterozygous mutation at exon 16 of the LDLR gene. HepG2 cells were treated with Cas9 mRNA and gRNA targeting the LDLR coding sequence (Dharmacon™, SG-011073-04) to generate indels. Single cell clones, including 1C10, were isolated from this bulk population and characterized by next-generation sequencing to confirm the presence of one wild-type allele and one disrupted allele predicted to form a premature stop codon. Total LDLR protein level was assessed by ELISA. Introduction of this mutation resulted in a decrease of LDLR protein expression level from about 27 μg/μg of input (HepG2 cells) to about 19 μg/μg of input (1C10 cells) (FIG. 25A). The flow cytometry-based cholesterol uptake assay as described in Example 3 was used to evaluate LDLR function in HepG2 and 1C10 cells. Introduction of this mutation resulted in a decrease of LDL-C uptake in 1C10 cells to about 0.6-fold, compared with HepG2 cells (FIG. 25B).

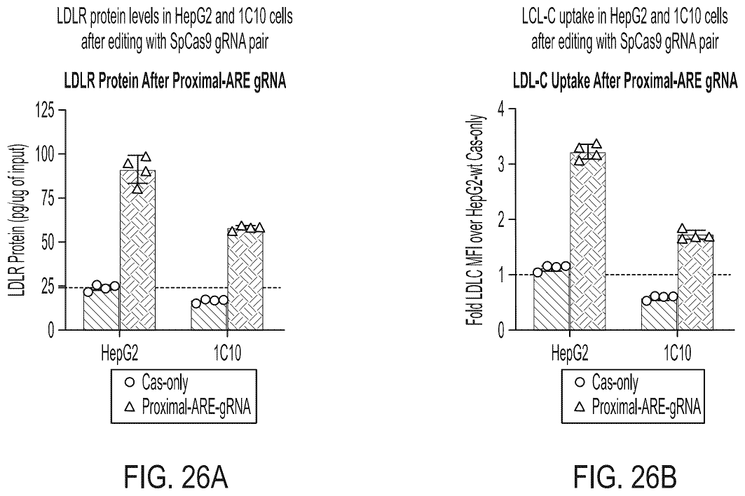

HepG2 and 1C10 cells were treated with a gRNA pair selected to induce the Proximal-ARE deletion (Target Sequence SEQ ID NOs: 73 and 300) encapsulated in LNPs with mRNA encoding SpCas9 (SEQ ID NO: 1480, CleanCap Cas9 mRNA from TriLink Biotechnologies). Cells were treated with LNPs for 4 days. Editing with SpCas9 gRNA pairs resulted in LDLR protein expression of up to about 90 μg/μg of input or about 58 μg/g of input in HepG2 or 1C10 cells, respectively (FIG. 26A). The amount of LDL-C taken up by edited HepG2 cells increased up to about 3.2-fold compared with unedited cells. The amount of LDL-C taken up by edited 1C10 cells increased up to about 1.7-fold compared with unedited cells (FIG. 26B).

### 8.13 Example 12: Determination of SpCas9 and AsCas12a Dual Guide Induction of Edits and Impact on Total Ldlr Expression in Wild-Type C57B/6 Mice

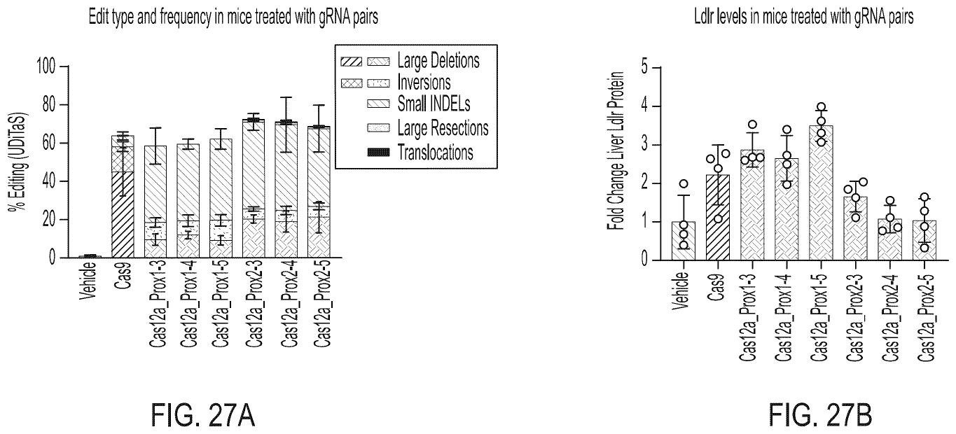

Surrogate gRNAs targeting mouse Ldlr were designed based on the human “Prox1” pair in Example 7 using either SpCas9 or AsCas12a (WT) (Tables 29A-29D). For targeting with Cas9, ALC0315 LNPs were formulated encapsulating mRNA encoding SpCas9 (SEQ ID NO: 1480) and both gRNAs in a 1:1:1 ratio by weight (Target Sequence SEQ ID NOs: 1317-1318). For targeting with Cas12a, ALC0315 LNPs were formulated containing mRNA encoding AsCas12a (SEQ ID NO: 899) and gRNAs with the following modifications: 5′ and 3′ terminal inverted dTs, and the “conservative 2′F in hairpin” pattern (SEQ ID NO: 35), (Target Sequence SEQ ID NOs: 1319-1323). LNPs were injected via intravenous tail vein injection into WT C57B/6 mice at 3 mg/kg. After 2 weeks, mice were euthanized and samples collected. Genomic DNA was extracted from subject mouse livers and analyzed via UDiTaS for frequency and type of edits induced at the 3′ UTR. Editing with the SpCas9 gRNA pair resulted in generation of large deletions or inversions in about 35-60% of cells. Editing with the AsCas12s (WT) gRNA pairs resulted in generation of large deletions or inversions in about 15-30% of cells (FIG. 27A).

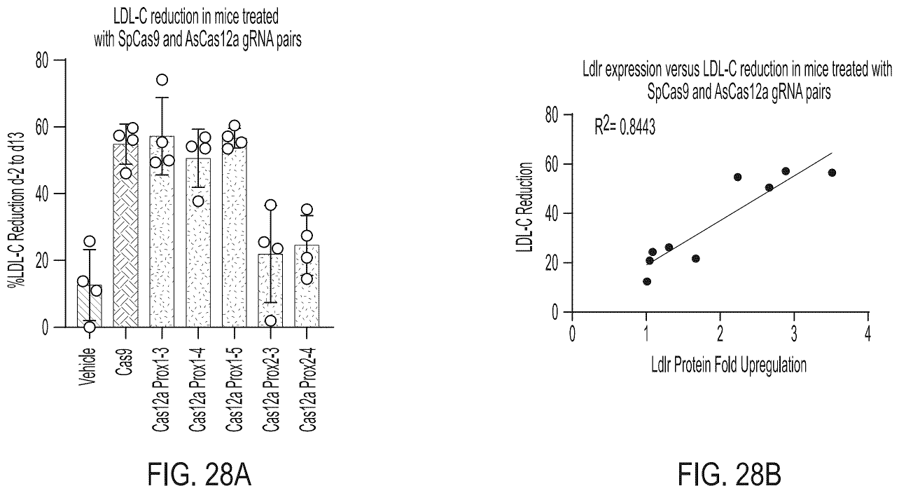

Protein extracted from subject mouse livers was analyzed for total Ldlr protein expression via a mouse-Ldlr-specific ELISA. Editing with the SpCas9 gRNA pair resulted in up to about a 2.2-fold change in total Ldlr protein expression. Editing with the AsCas12a (WT) gRNA pairs resulted in up to about a 3.5-fold change in total Ldlr protein expression (FIG. 27B). Serum collected on experimental day 2 and day 13 was analyzed for LDL-C levels using a BioTechne LDL-C assay kit (Catalog No. NBP3-25881). Editing with either the SpCas9 gRNA pair or the AsCas12a gRNA pairs resulted in up to about a 60% LDL-C reduction at day 13 compared with day 2 (FIG. 28A). Ldlr total protein fold upregulation correlated to LDL-C percent reduction with R2=0.8443 (FIG. 28B).

TABLE 29A
Ldlr Target Sequences in C57B/6 mouse

| Guide | SEQ | |
|---|---|---|
| Nickname | ID NO | Target Sequence (DNA) |
| Cas9 (5′) | 1317 | TTGAAATTCGAATTCTGCCT |
| Cas9 (3′) | 1318 | GTGCGTTGCTTTGAGTGGGT |
| Cas12a 1 | 1319 | TTGTCTGGGGACAGAAAAGGC |
| Cas12a 2 | 1320 | GCTTCCAAGGAAATGAAGATG |
| Cas12a 3 | 1321 | AATGGGCTGATCTCAGGCCTG |
| Cas12a 4 | 1322 | AAAGCAGTTCCCACCCACTCA |
| Cas12a 5 | 1323 | AGTGGGTGGGAACTGCTTTGA |

TABLE 29B
gRNA Targeting Domains for Ldlr in C57B/6 mouse

| Guide | SEQ ID | |
|---|---|---|
| Nickname | NO | gRNA Targeting Domain (RNA) |
| Cas9 (5′) | 1464 | mU*mU*mG*rArArArUrUrCrGrArArUrUrCrUrGrCrCrU |
| Cas9 (3′) | 1466 | mG*mU*mG*rCrGrUrUrGrCrUrUrUrGrArGrUrGrGrGrU |
| Cas12a 1 | 1326 | rUrUrGrUrCrUrGrGrGrGrArCrArGrArArArArGrGrC |
| Cas12a 2 | 1327 | rGrCrUrUrCrCrArArGrGrArArArUrGrArArGrArUrG |
| Cas12a 3 | 1328 | rArArUrGrGrGrCrUrGrArUrCrUrCrArGrGrCrCrUrG |
| Cas12a 4 | 1329 | rArArArGrCrArGrUrUrCrCrCrArCrCrCrArCrUrCrA |
| Cas12a 5 | 1330 | rArGrUrGrGrGrUrGrGrGrArArCrUrGrCrUrUrUrGrA |

TABLE 29C
gRNA Sequences for Ldlr in C57B/6 mouse

| Guide | SEQ ID | |
|---|---|---|
| Nickname | NO | gRNA Sequence |
| Cas9 (5′) | 1472 | mU*mU*mG*rArArArUrUrCrGrArArUrUrCrUrGrCrCrUrGr |
| | | UrUrUrUrArGrAmGmCmUmAmGmAmAmAmUmAmGmCr |
| | | ArArGrUrUrArArArArUrArArGrGrCrUrArGrUrCrCrGrUrUr |
| | | ArUrCrAmAmCmUmUmGmAmAmAmAmAmGmUmGmG |
| | | mCmAmCmCmGmAmGmUmCmGmGmUmGmCmU*mU* |
| | | mU*mU |
| Cas9 (3′) | 1473 | mG*mU*mG*rCrGrUrUrGrCrUrUrUrGrArGrUrGrGrGrUrGr |
| | | UrUrUrUrArGrAmGmCmUmAmGmAmAmAmUmAmGmCr |
| | | ArArGrUrUrArArArArUrArArGrGrCrUrArGrUrCrCrGrUrUr |
| | | ArUrCrAmAmCmUmUmGmAmAmAmAmAmGmUmGmG |
| | | mCmAmCmCmGmAmGmUmCmGmGmUmGmCmU*mU* |
| | | mU*mU |
| Cas12a 1 | 1333 | rUrArArUrUrUrCrUrArCrUrCrUrUrGrUrArGrArUrUrUrGrU |
| | | rCrUrGrGrGrGrArCrArGrArArArArGrGrC |
| Cas12a 2 | 1334 | rUrArArUrUrUrCrUrArCrUrCrUrUrGrUrArGrArUrGrCrUrUr |
| | | CrCrArArGrGrArArArUrGrArArGrArUrG |
| Cas12a 3 | 1335 | rUrArArUrUrUrCrUrArCrUrCrUrUrGrUrArGrArUrArArUrG |
| | | rGrGrCrUrGrArUrCrUrCrArGrGrCrCrUrG |
| Cas12a 4 | 1336 | rUrArArUrUrUrCrUrArCrUrCrUrUrGrUrArGrArUrArArArG |
| | | rCrArGrUrUrCrCrCrArCrCrCrArCrUrCrA |
| Cas12a 5 | 1337 | rUrArArUrUrUrCrUrArCrUrCrUrUrGrUrArGrArUrArGrUrG |
| | | rGrGrUrGrGrGrArArCrUrGrCrUrUrUrGrA |

TABLE 29D
Target Sequence Pairs for Mouse Experiments

| | 5′ Guide | 3′ Guide |
|---|---|---|
| Pair Nickname | SEQ ID NO | SEQ ID NO |
| Cas9 | 1317 | 1318 |
| Cas12a_Prox1-3 | 1319 | 1321 |
| Cas12a_Prox1-4 | 1319 | 1322 |
| Cas12a_Prox1-5 | 1319 | 1323 |
| Cas12a_Prox2-3 | 1320 | 1321 |
| Cas12a_Prox2-4 | 1320 | 1322 |
| Cas12a_Prox2-5 | 1320 | 1323 |

### 8.14 Example 13: Determination of SpCas9 Proximal and Proximal-ARE Dual Guide Impact on Induction of Edits, Total Ldlr Expression, Blood LDL-C Levels, and Blood ApoB Levels in Wild-Type Mice

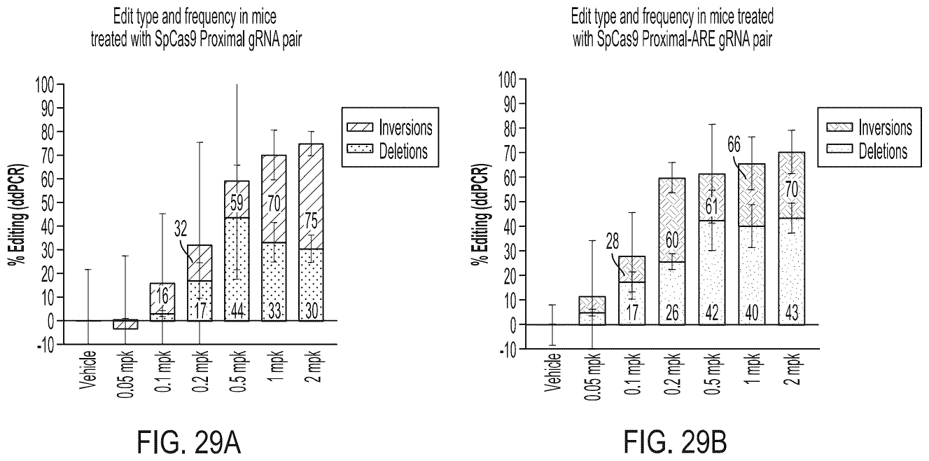

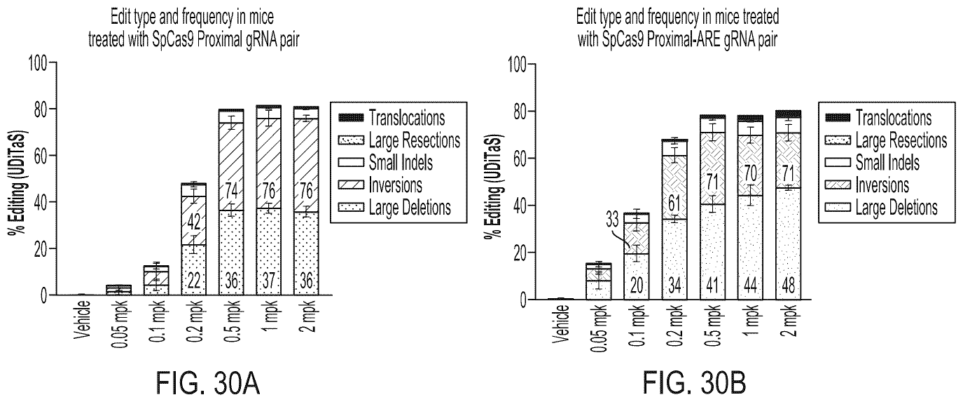

Surrogate gRNAs targeting mouse Ldlr were designed to induce a Proximal-like and a Proximal-ARE-like deletion using SpCas9 (Tables 31A-31B). ALC0315 LNPs were formulated encapsulating mRNA encoding SpCas9 (SEQ ID NO: 1480) and gRNAs (gRNA Sequences SEQ ID NOs: 1463, 1465, and 1467, see Table 31C). LNPs were injected via intravenous tail vein injection into WT C57B/6 mice at 0 mg/kg (vehicle control) or 0.05-5 mg/kg (RNA-LNP) (Table 30). After 2 weeks, mice were euthanized and samples collected. Genomic DNA was extracted from subject mouse whole-liver homogenate and analyzed via RT-ddPCR and Next-Generation Sequencing (NGS) for frequency and type of edits induced at the 3′ UTR. Increasing total RNA concentrations for each gRNA pair resulted in increased generation of functional edits in mouse liver cells as assessed by both UDiTaS and NGS (FIGS. 29A-B and FIGS. 30A-B). Functional editing (i.e., the sum of large deletions and inversions) was achieved in at least about 60% of cells edited with the Proximal gRNA pair at >0.5 mg/kg. Editing was detectable at >0.1 mg/kg (FIG. 29A and FIG. 30A). Functional editing (i.e., the sum of large deletions and inversions) was achieved in at least about 60% of cells edited with the Proximal-ARE gRNA pair at ≥0.2 mg/kg. Editing was detectable at ≥0.05 mg/kg (FIG. 29B and FIG. 30B).

TABLE 30
Mouse study design

| | | IVT | | | | | | |
|---|---|---|---|---|---|---|---|---|
| | | mRNA | 5′ Target | 3′ Target | | | | |
| | | SEQ ID | Sequence | Sequence | Dose | Mice | | Endpoint |
| Group | Description | NO: | SEQ ID NO | SEQ ID NO | (mg/kg) | (42 total) | Time points | assays |
| 1 | Proximal | N/A | N/A | N/A | 0 | 6 (3F/3M) | Fasted serum | 1. Whole Liver |
| | vs. Proximal-ARE | 1480 | 1317 vs. | 1318 vs. | 0.05 | 6 (3F/3M) | sample Day 4 | lobe |
| 2 | Proximal | | 1461 | 1318 | | | (Pre-dose) Day 6 | 2. Liver punch |
| | vs. Proximal-ARE | 1480 | 1317 vs. | 1318 vs. | 0.1 | 6 (3F/3M) | (Post-dose) | (UDiTaS) |
| 3 | Proximal | | 1461 | 1318 | | | Takedown with | 3. Liver Punch |
| | vs. Proximal-ARE | | | | | | Liver Collection | (Protein ELISA) |
| 4 | Proximal | 1480 | 1317 vs. | 1318 vs. | 0.2 | 6 (3F/3M) | (Day 7) | 4. Fasted Pre- vs |
| | vs. Proximal-ARE | | 1461 | 1318 | | | | Post-dose Serum |
| 5 | Proximal | 1480 | 1317 vs. | 1318 vs. | 0.5 | 6 (3F/3M) | | (Protein ELISA) |
| | vs. Proximal-ARE | | 1461 | 1318 | | | | |
| 6 | Proximal | 1480 | 1317 vs. | 1318 vs. | 1 | 6 (3F/3M) | | |
| | vs. Proximal-ARE | | 1461 | 1318 | | | | |
| 7 | Proximal | 1480 | 1317 vs. | 1318 vs. | 2 | 6 (3F/3M) | | |
| | vs. Proximal-ARE | | 1461 | 1318 | | | | |

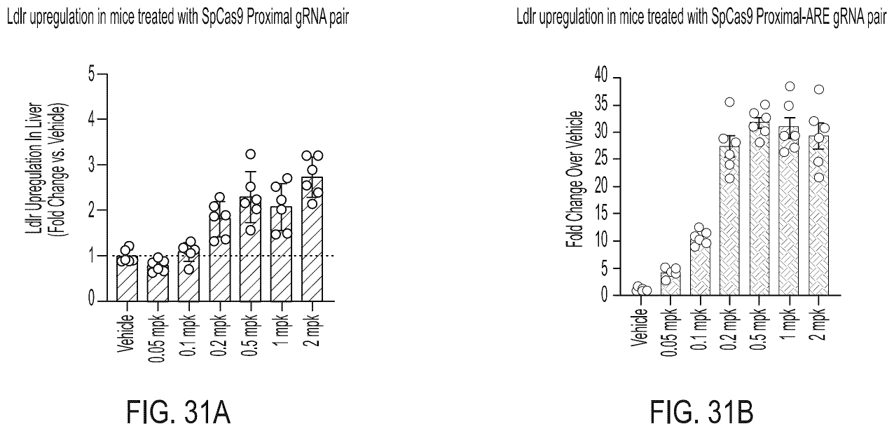

Increasing total RNA concentrations for each gRNA also resulted in larger fold increases in Ldlr total protein extracted from subject mouse livers as assessed by a mouse-Ldlr-specific ELISA. Editing with the Proximal SpCas9 gRNA pair resulted in up to about a 2.7-fold change in total Ldlr protein expression (FIG. 31A). Editing with the Proximal-ARE SpCas9 gRNA pair resulted a maximum of up to a 30-fold increase in total Ldlr protein expression and up to about a 3.4-fold increase in total Ldlr protein expression was detected upon treatment with 0.05 mg/kg LNP (FIG. 31B).

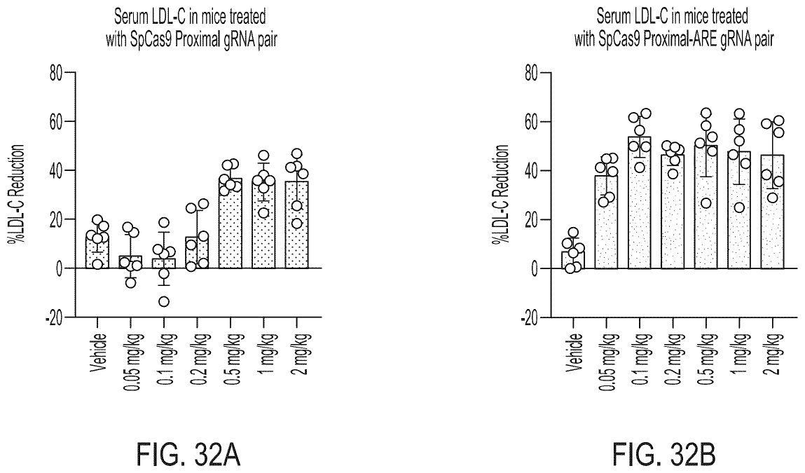

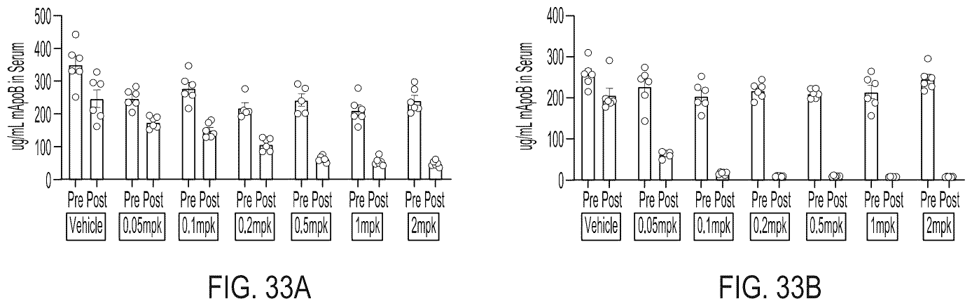

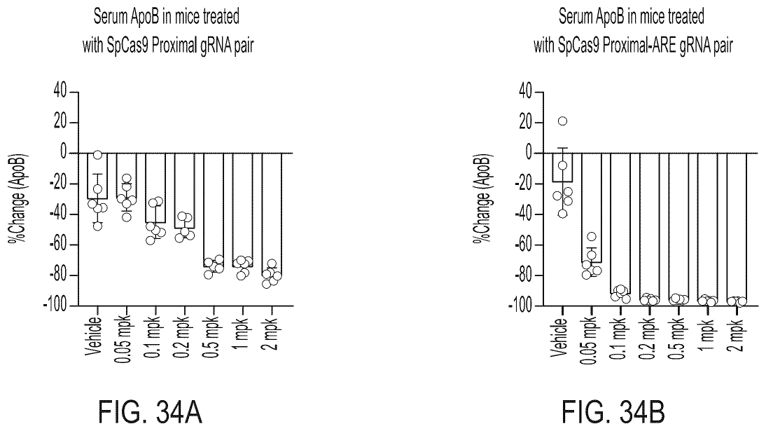

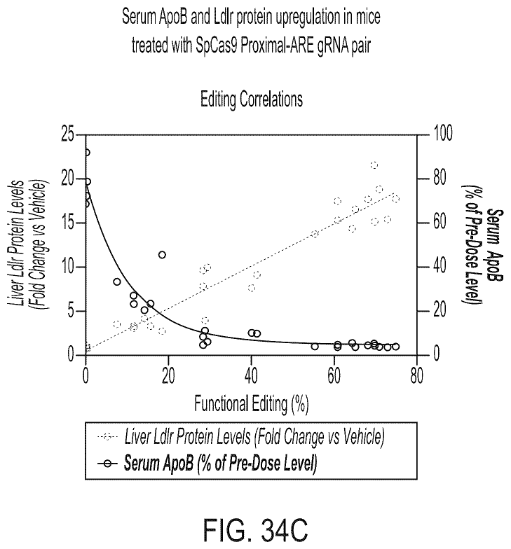

Serum collected on experimental day 2 and day 13 was analyzed for LDL-C levels using a BioTechne LDL-C assay kit (Catalog No. NBP3-25881). Editing with the Proximal SpCas9 gRNA pair resulted in up to about a 35% reduction in blood LDL-C at day 13 compared with day 2 (FIG. 32A). Editing with the Proximal-ARE SpCas9 gRNA pair resulted in a maximum of up to about a 54% LDL-C reduction at day 13 compared with day 2, and up to about a 38% reduction in serum LDL-C was detected upon treatment with 0.05 mg/kg LNP (FIG. 32B). Editing with the Proximal SpCas9 gRNA pair resulted in up to about an 80% reduction in serum ApoB compared with pre-treatment ApoB levels (FIG. 33A and FIG. 34A). Editing with the Proximal-ARE SpCas9 gRNA pair resulted in a maximum of up to about a 97% serum ApoB reduction compared with pre-treatment ApoB levels, and up to about a 71% reduction in serum ApoB levels was detected upon treatment with 0.05 mg/kg LNP (FIG. 33B and FIG. 34B). Increased functional editing correlated with both Ldlr protein upregulation and ApoB reduction (FIG. 34C, Table 32).

TABLE 31A
Ldlr Target Sequence Pairs in C57B/6 mouse

| | Relative | SEQ ID | |
|---|---|---|---|
| Pair Nickname | Guide Position | NO | Target Sequence (DNA) |
| SpCas9 Proximal | 5′ | 1317 | TTGAAATTCGAATTCTGCCT |
| | 3′ | 1318 | GTGCGTTGCTTTGAGTGGGT |
| SpCas9 Proximal- | 5′ | 1461 | GAGCAACACAGAGCCCAGGG |
| ARE | 3′ | 1318 | GTGCGTTGCTTTGAGTGGGT |

TABLE 31B
gRNA Targeting Domains for Ldlr in C57B/6 Mouse

| | Relative | | |
|---|---|---|---|
| Pair | Guide | SEQ ID | |
| Nickname | Position | NO | gRNA Targeting Domain (RNA) |
| SpCas9 | 5′ | 1464 | mU*mU*mG*rArArArUrUrCrGrArArUrUrCrUrGrC |
| Proximal | | | rCrU |
| | 3′ | 1466 | mG*mU*mG*rCrGrUrUrGrCrUrUrUrGrArGrUrGrG |
| | | | rGrU |
| SpCas9 | 5′ | 1462 | mG*mA*mG*rCrArArCrArCrArGrArGrCrCrCrArG |
| Proximal- | | | rGrG |
| ARE | 3′ | 1466 | mG*mU*mG*rCrGrUrUrGrCrUrUrUrGrArGrUrGrG |
| | | | rGrU |

TABLE 31C
gRNA Sequences for Ldlr in C57B/6 Mouse

| | Relative | | |
|---|---|---|---|
| Pair | Guide | SEQ ID | |
| Nickname | Position | NO | gRNA Sequence |
| SpCas9 | 5′ | 1465 | mU*mU*mG*rArArArUrUrCrGrArArUrUrCrUrGrCrCr |
| Proximal | | | UrGrUrUrUrUrArGrAmGmCmUmAmGmAmAmAmU |
| | | | mAmGmCrArArGrUrUrArArArArUrArArGrGrCrUrAr |
| | | | GrUrCrCrGrUrUrArUrCrAmAmCmUmUmGmAmAmA |
| | | | mAmAmGmUmGmGmCmAmCmCmGmAmGmUmCm |
| | | | GmGmUmGmCmU*mU*mU*mU |
| | 3′ | 1467 | mG*mU*mG*rCrGrUrUrGrCrUrUrUrGrArGrUrGrGrGr |
| | | | UrGrUrUrUrUrArGrAmGmCmUmAmGmAmAmAmU |
| | | | mAmGmCrArArGrUrUrArArArArUrArArGrGrCrUrAr |
| | | | GrUrCrCrGrUrUrArUrCrAmAmCmUmUmGmAmAmA |
| | | | mAmAmGmUmGmGmCmAmCmCmGmAmGmUmCm |
| | | | GmGmUmGmCmU*mU*mU*mU |
| SpCas9 | 5′ | 1463 | mG*mA*mG*rCrArArCrArCrArGrArGrCrCrCrArGrGr |
| Proximal- | | | GrGrUrUrUrUrArGrAmGmCmUmAmGmAmAmAmU |
| ARE | | | mAmGmCrArArGrUrUrArArArArUrArArGrGrCrUrAr |
| | | | GrUrCrCrGrUrUrArUrCrAmAmCmUmUmGmAmAmA |
| | | | mAmAmGmUmGmGmCmAmCmCmGmAmGmUmCm |
| | | | GmGmUmGmCmU*mU*mU*mU |
| | 3′ | 1467 | mG*mU*mG*rCrGrUrUrGrCrUrUrUrGrArGrUrGrGrGr |
| | | | UrGrUrUrUrUrArGrAmGmCmUmAmGmAmAmAmU |
| | | | mAmGmCrArArGrUrUrArArArArUrArArGrGrCrUrAr |
| | | | GrUrCrCrGrUrUrArUrCrAmAmCmUmUmGmAmAmA |
| | | | mAmAmGmUmGmGmCmAmCmCmGmAmGmUmCm |
| | | | GmGmUmGmCmU*mU*mU*mU |

All bases are in upper case. Lowercase “r” represents RNA, 2′-hydroxy; bases not modified by an “r” are DNA. Lowercase “m” represents RNA with a 2′-methyl group
All bases are linked via standard phosphodiester bonds except as noted:
represents phosphorothioate modification

TABLE 32
Comparison of deleted regions and Cas effectors in mouse studies

| | | | | | Dose |
|---|---|---|---|---|---|
| | | Functional | Ldlr | | required |
| | | Editing | protein | | for max |
| | Cas | (deletions + | upreg- | LDL-C | reduction |
| Deletion | effector | inversions) | ulation | reduction | (mg/kg) |
| ADIST1 | AsCas12a | — | — | — | |
| Proximal | AsCas12a | 25% | 1.25X | 26% | Tested |
| | | | | | only 3 |
| | SpCas9 | 58% | ~2.5X | 35-55% | 0.5 or 3 |
| Proximal- | AsCas12a | — | — | — | |
| ARE | SpCas9 | 70% | >17X | 55% | 0.1 |

### 8.15 Example 14: Determination of SpCas9 Proximal-ARE Dual Guide Impact on Induction of Functional Edits, Total Ldlr Protein Expression, and Blood ApoB Levels in Wild-Type and Ldlr^{+/-} Mice

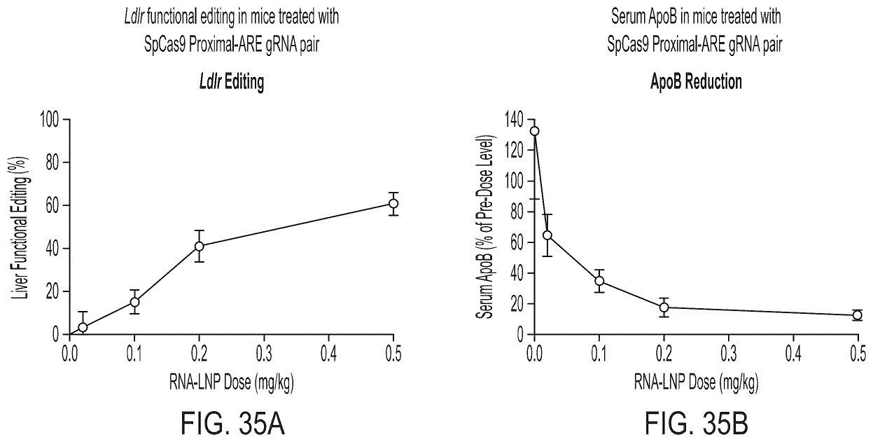

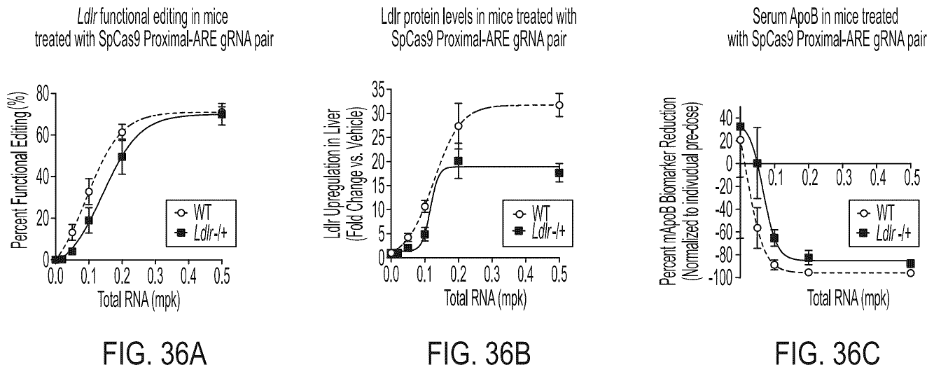

ALC0315 LNPs were formulated encapsulating mRNA encoding SpCas9 (SEQ ID NO: 1480) and two gRNAs (Target Sequence SEQ ID NO: 1318 and 1461, gRNA targeting domain SEQ ID NO: 1462 and 1466, gRNA SEQ ID NO: 1463 and 1467, see Tables 31A-31C). Heterozygous Ldlr^{+/-} mice were generated by breeding B6; 129S7-Ldlr^{tm/Her}/J mice (The Jackson Laboratory™) with wild-type C57BL/6 mice. LNPs were injected via intravenous tail vein injection into wild-type C57B/6 and Ldlr^{+/-} mice at 3 mg/kg (RNA-LNP). After 7 days, mice were euthanized and samples collected. Genomic DNA was extracted from subject mouse whole-liver homogenate and analyzed via UDiTaS for frequency and type of edits induced at the 3′ UTR. Increasing total RNA concentrations resulted in increased generation of functional edits (i.e., the sum of large deletions and inversions) in up to about 70% of wild-type mouse liver cells and up to about 60% of Ldlr^{-/-} mouse liver cells (FIG. 35A and FIG. 36A).

Ldlr total protein extracted from subject mouse livers was assessed by a mouse-Ldlr-specific ELISA. Editing with the Proximal-ARE SpCas9 gRNA pair (Target Sequences SEQ ID NOs: 1318 and 1461) resulted in up to about a 30-fold change in total Ldlr protein levels in wild-type mice and up to about a 10-fold change in total Ldlr protein levels in Ldlr^{+/-} mice (FIG. 36B).

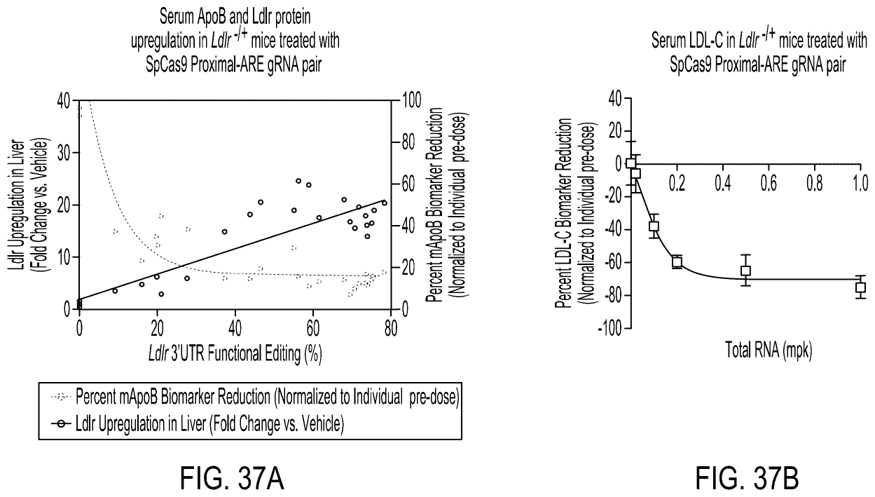

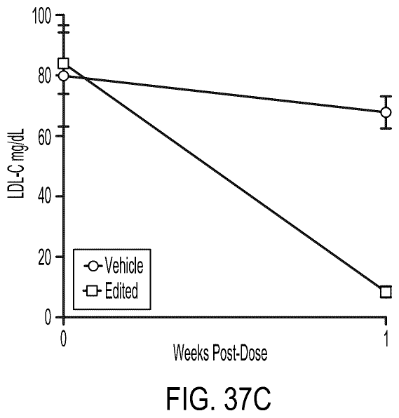

Editing with the Proximal-ARE SpCas9 gRNA pair resulted in a serum ApoB reduction of up to about 97% in wild-type mice and up to about 90% in Ldlr^{+/-} mice compared with pre-treatment ApoB levels (FIGS. 35B and 36C). Increased functional editing correlated with both Ldlr protein upregulation and ApoB reduction (FIG. 37A).

Serum collected at pre-dose and day 7 timepoints was analyzed for LDL-C levels using an enzymatic LDL-C assay kit (BioTechne Catalog No. NBP3-25881). Editing with the Proximal-ARE SpCas9 gRNA pair resulted in a maximum of up to about a 70% LDL-C reduction at day 7 compared with predose, and up to about a 40% reduction in serum LDL-C was detected upon treatment with 0.1 mg/kg LNP (FIG. 37B). Enzymatic LDL-C assays could have background signal from other lipoproteins. Therefore, an HPLC-based LDL-C quantification method was developed in house and >90% LDL-C reduction was achieved in Ldlr^{+/-} mice at 1 mpk compared to pre-dose (FIG. 37C).

### 8.16 Example 15: Administration of SpCas9 Proximal-ARE Dual Guides to Wild-Type and Ldlr^{+/-} Mice on Regular and High-Fat Diet

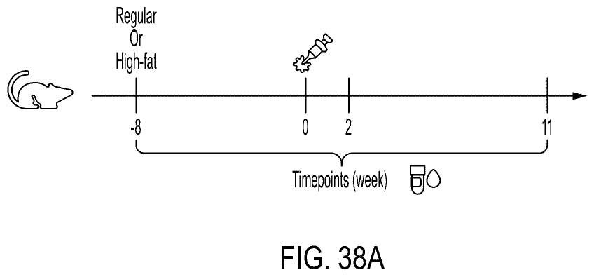

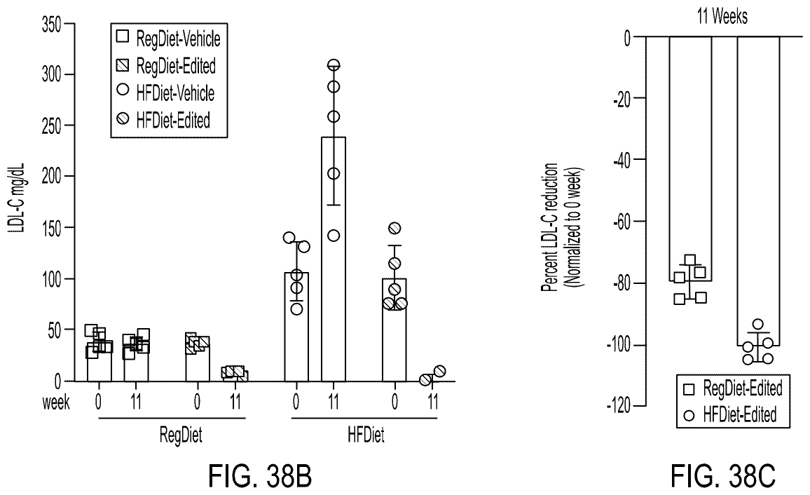

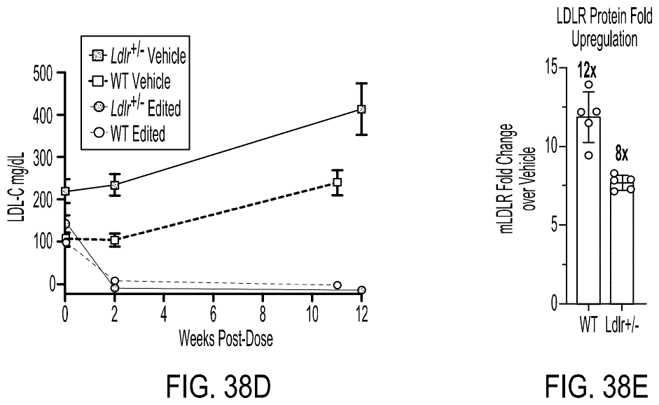

ALC0315 LNPs were formulated encapsulating mRNA encoding SpCas9 (SEQ ID NO: 1480) and two gRNAs (Target Sequence SEQ ID NO: 1318 and 1461, gRNA targeting domain SEQ ID NO: 1462 and 1466, gRNA SEQ ID NO: 1463 and 1467, see Tables 31A-31C). Male WT C57B/6 and Ldlr^{+/-} mice were fed a regular or high-fat diet for 8 weeks, and then treated with LNPs via intravenous tail injection at 0 mg/kg (vehicle control) or 1 mg/kg (RNA-LNP) (FIG. 38A). Serum collected pre-dosing (week 0) and at week 11 or week 12 was analyzed for LDL-C levels using an in-house developed HPLC assay. A high-fat diet resulted in a maximum increase in serum LDL-C levels of about 6.5-fold in WT mice (FIG. 38B). Editing with the Proximal-ARE SpCas9 gRNA pair resulted in a reduction in serum LDL-C of up to about 80% and up to about 100% in WT mice fed a regular or a high-fat diet, respectively at the 11 week or 12 week time point normalized to the 0 week time point. (FIG. 38C and FIG. 38D).

Ldlr^{+/-} mice were generated by breeding B6; 129S7-LdlrtmlHer/J mice (The Jackson Laboratory™) with wild-type C57BL/6 mice.

Protein extracted from subject mouse livers was analyzed for total LDLR protein expression via a mouse-Ldlr-specific ELISA. Editing with the SpCas9 Proximal-ARE gRNA pair resulted in up to about a 12-fold increase in total LDLR protein expression in wild-type mice and up to about an 8-fold increase in total LDLR protein expression in Ldlr^{+/-} mice (FIG. 38E).

### 8.17 Example 16: Determination of SpCas9 and AsCas12a Dual Guide Induction of Edits and Impact on LDLR mRNA and Total LDLR Protein Expression in Cynomolgus Hepatocytes

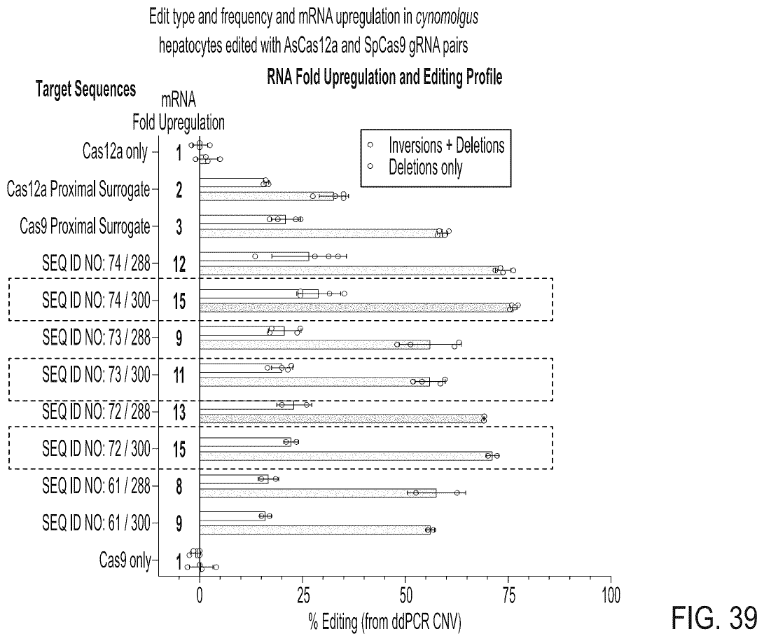

A plurality of SpCas9 and AsCas12a gRNAs targeting human LDLR with non-human primate (NHP) cross-reactivity were selected for study in cynomolgus hepatocytes. Cells were treated with 10-2 mg/mL total RNA (i.e., the sum of nuclease mRNA and two guide RNAs) at a 1:1:1 ratio of SpCas9 or AsCas12a mRNA: first gRNA: second gRNA LNPs for 4 days. For targeting with SpCas9, ALC0315 LNPs were formulated encapsulating mRNA encoding SpCas9 (SEQ ID NO: 1480) and gRNAs (5′ Target Sequence SEQ ID NOs: 61, 72, 73, 74, and 79. 3′ Target Sequence SEQ ID NOs: 288, 299, and 300, Tables 33A-33C). SpCas9 gRNAs were manufactured by Axolabs™, with the modification patterns shown in Table 33C. For the AsCas12a gRNA pair, hepatocytes were treated with LNPs containing mRNA encoding AsCas12a (SEQ ID NO: 899) and gRNAs with the following modifications: 5′ and 3′ terminal inverted dTs, and the “conservative 2′F in hairpin” pattern (SEQ ID NO: 35), with Target Sequences of SEQ ID NO 553 and 589 (Table 33C). For the SpCas9 gRNA pair, hepatocytes were treated with LNPs containing mRNA encoding SpCas9 (SEQ ID NO: 1480, CleanCap Cas9 mRNA from TriLink Biotechnologies) and the gRNA pair. SpCas9 gRNA pairs inducing the Proximal-ARE mutation increased LDLR mRNA levels by up to 15-fold and induced functional edits in up to about 75% of cells, as determined by ddPCR (FIG. 39).

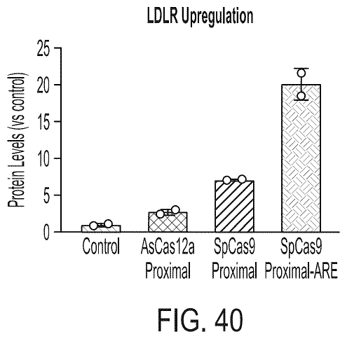

Three gRNAs were analyzed further: an SpCas9 gRNA pair inducing the Proximal mutation, an SpCas9 gRNA pair inducing the Proximal-ARE mutation, and an AsCas12a gRNA pair inducing the Proximal mutation (Table 33C). Total LDLR levels were quantified by ELISA. Editing with the AsCas12a gRNA pair inducing the Proximal mutation resulted in LDLR protein levels increasing by up to about 3-fold compared with a non-editing control. Editing with the SpCas9 gRNA pair inducing the Proximal mutation resulted in LDLR protein levels increasing by up to about 5-fold. Editing with the SpCas9 gRNA pair inducing the Proximal-ARE mutation increased LDLR protein levels by up to about 20-fold (FIG. 40).

TABLE 33A
Target Sequence Pairs for Cynomolgus Hepatocyte Experiments

| | Relative | | |
|---|---|---|---|
| Pair | Guide | SEQ | |
| Nickname | Position | ID NO | Sequence (DNA) |
| AsCas12a | 5′ | 553 | TCTTATCCTTCACGAGGAAAG |
| Proximal | 3′ | 589 | GGAGAATGATGTCCCCGTTGT |
| Surrogate | | | |
| SpCas9 | 5′ | 79 | CAGTGCCCATGCAATGGCTT |
| Proximal | 3′ | 299 | ATGAGTGGCTTCTGGGAGAT |
| Surrogate | | | |
| SpCas9 | 5′ | 73 | ATATAAAACAAAGCTCTGGC |
| Proximal- | 3′ | 300 | TACAAAAACCTTCTATACAG |
| ARE | | | |
| 74/288 | 5′ | 74 | AAATATATAAAACAAAGCTC |
| | 3′ | 288 | AATCTTTGTACATAAAGAAG |
| 74/300 | 5′ | 74 | AAATATATAAAACAAAGCTC |
| | 3′ | 300 | TACAAAAACCTTCTATACAG |
| 73/288 | 5′ | 73 | ATATAAAACAAAGCTCTGGC |
| | 3′ | 288 | AATCTTTGTACATAAAGAAG |
| 72/288 | 5′ | 72 | AGCTCTGGCAGGCAATGCTT |
| | 3′ | 288 | AATCTTTGTACATAAAGAAG |
| 72/300 | 5′ | 72 | AGCTCTGGCAGGCAATGCTT |
| | 3′ | 300 | TACAAAAACCTTCTATACAG |
| 61/288 | 5′ | 61 | ACGTGGCGTGAACATCTGCC |
| | 3′ | 288 | AATCTTTGTACATAAAGAAG |
| 61/300 | 5′ | 61 | ACGTGGCGTGAACATCTGCC |
| | 3′ | 300 | TACAAAAACCTTCTATACAG |

TABLE 33B
Targeting Domain Sequence Pairs for Cynomolgus Hepatocyte Experiments

| | Relative | | |
|---|---|---|---|
| Pair | Guide | SEQ | |
| Nickname | Position | ID NO | Sequence (RNA) |
| AsCas12a | 5′ | 611 | rUrCrUrUrArUrCrCrUrUrCrArCrGrArGrGr |
| Proximal | | | ArArArG |
| Surrogate | 3′ | 647 | rGrGrArGrArArUrGrArUrGrUrCrCrCrCrGr |
| | | | UrUrGrU |
| SpCas9 | 5′ | 1455 | mC*mA*mG*rUrGrCrCrCrArUrGrCrArArU |
| Proximal | | | rGrGrCrUrU |
| Surrogate | 3′ | 1456 | mA*mU*mG*rArGrUrGrGrCrUrUrCrUrGrG |
| | | | rGrArGrArU |
| SpCas9 | 5′ | 1452 | mA*mU*mA*rUrArArArArCrArArArGrCrU |
| Proximal- | | | rCrUrGrGrC |
| ARE | 3′ | 1475 | mU*mA*mC*rArArArArArCrCrUrUrCrUrA |
| | | | rUrArCrArG |
| 74/288 | 5′ | 1453 | mA*mA*mA*rUrArUrArUrArArArArCrAr |
| | | | ArArGrCrUrC |
| | 3′ | 1474 | mA*mA*mU*rCrUrUrUrGrUrArCrArUrArA |
| | | | rArGrArArG |
| 74/300 | 5′ | 1453 | mA*mA*mA*rUrArUrArUrArArArArCrAr |
| | | | ArArGrCrUrC |
| | 3′ | 1475 | mU*mA*mC*rArArArArArCrCrUrUrCrUrA |
| | | | rUrArCrArG |
| 73/288 | 5′ | 1452 | mA*mU*mA*rUrArArArArCrArArArGrCrU |
| | | | rCrUrGrGrC |
| | 3′ | 1474 | mA*mA*mU*rCrUrUrUrGrUrArCrArUrArA |
| | | | rArGrArArG |
| 72/288 | 5′ | 1451 | mA*mG*mC*rUrCrUrGrGrCrArGrGrCrArA |
| | | | rUrGrCrUrU |
| | 3′ | 1474 | mA*mA*mU*rCrUrUrUrGrUrArCrArUrArA |
| | | | rArGrArArG |
| 72/300 | 5′ | 1451 | mA*mG*mC*rUrCrUrGrGrCrArGrGrCrArA |
| | | | rUrGrCrUrU |
| | 3′ | 1475 | mU*mA*mC*rArArArArArCrCrUrUrCrUrA |
| | | | rUrArCrArG |
| 61/288 | 5′ | 1454 | mA*mC*mG*rUrGrGrCrGrUrGrArArCrArU |
| | | | rCrUrGrCrC |
| | 3′ | 1474 | mA*mA*mU*rCrUrUrUrGrUrArCrArUrArA |
| | | | rArGrArArG |
| 61/300 | 5′ | 1454 | mA*mC*mG*rUrGrGrCrGrUrGrArArCrArU |
| | | | rCrUrGrCrC |
| | 3′ | 1475 | mU*mA*mC*rArArArArArCrCrUrUrCrUrA |
| | | | rUrArCrArG |

All bases are in upper case. Lowercase “r” represents RNA, 2′-hydroxy; bases not modified by an ″r” are DNA. Lowercase “m” represents RNA with a 2′-methyl group.
All bases are linked via standard phosphodiester bonds except as noted:
“*” represents phosphorothioate modification.

TABLE 33C
gRNA Pairs for Cynomolgus Hepatocyte Experiments

| | Relative | | |
|---|---|---|---|
| Pair | Guide | SEQ | |
| Nickname | Position | ID NO | Sequence (RNA) |
| AsCas12a | 5′ | 1457 | /5InvdT/ATGTGTTTTTGTCAAAAGACCT |
| Proximal | | | TTTrUrArArUrUrUrUrArCrUrCrUrUrGrUr |
| Surrogate | | | ArGrArUrUrCrUrUrArUrCrCrUrUrCrArCrG |
| | | | rArGrGrArArArG/3InvdT/ |
| | 3′ | 1458 | /InvdT/ATGTGTTTTTGTCAAAAGACCTT |
| | | | TTrUrArArUrUrUrCrUrArCrUrCrUrUrGrUr |
| | | | ArGrArUrGrGrArGrArArUrGrArUrGrUrCrC |
| | | | rCrCrGrUrUrGrU/InvdT/ |
| SpCas9 | 5′ | 1459 | mC*mA*mG*rUrGrCrCrCrArUrGrCrArArU |
| Proximal | | | rGrGrCrUrUrGrUrUrUrUrArGrAmGmCmU |
| Surrogate | | | mAmGmAmAmAmUmAmGmCrArArGrUr |
| | | | UrArArArArUrArArGrGrCrUrArGrUrCrCrG |
| | | | rUrUrArUrCrAmAmCmUmUmGmAmAmA |
| | | | mAmAmGmUmGmGmCmAmCmCmGmA |
| | | | mGmUmCmGmGmUmGmCmU*mU*mU* |
| | | | mU |
| | 3′ | 1460 | mA*mU*mG*rArGrUrGrGrCrUrUrCrUrGrG |
| | | | rGrArGrArUrGrUrUrUrUrArGrAmGmCmU |
| | | | mAmGmAmAmAmUmAmGmCrArArGrUr |
| | | | UrArArArArUrArArGrGrCrUrArGrUrCrCrG |
| | | | rUrUrArUrCrAmAmCmUmUmGmAmAmA |
| | | | mAmAmGmUmGmGmCmAmCmCmGmA |
| | | | mGmUmCmGmGmUmGmCmU*mU*mU* |
| | | | mU |
| SpCas9 | 5′ | 1477 | mA*mU*mA*rUrArArArArCrArArArGrCrU |
| Proximal- | | | rCrUrGrGrCrGrUrUrUrUrArGrAmGmCmU |
| ARE | | | mAmGmAmAmAmUmAmGmCrArArGrUr |
| | | | UrArArArArUrArArGrGrCrUrArGrUrCrCrG |
| | | | rUrUrArUrCrAmAmCmUmUmGmAmAmA |
| | | | mAmAmGmUmGmGmCmAmCmCmGmA |
| | | | mGmUmCmGmGmUmGmCmU*mU*mU* |
| | | | mU |
| | 3′ | 1450 | mU*mA*mC*rArArArArArCrCrUrUrCrUrA |
| | | | rUrArCrArGrGrUrUrUrUrArGrAmGmCmU |
| | | | mAmGmAmAmAmUmAmGmCrArArGrUr |
| | | | UrArArArArUrArArGrGrCrUrArGrUrCrCrG |
| | | | rUrUrArUrCrAmAmCmUmUmGmAmAmA |
| | | | mAmAmGmUmGmGmCmAmCmCmGmA |
| | | | mGmUmCmGmGmUmGmCmU*mU*mU* |
| | | | mU |
| 74/288 | 5′ | 1478 | mA*mA*mA*rUrArUrArUrArArArArCrAr |
| | | | ArArGrCrUrCrGrUrUrUrUrArGrAmGmCm |
| | | | UmAmGmAmAmAmUmAmGmCrArArGrU |
| | | | rUrArArArArUrArArGrGrCrUrArGrUrCrCr |
| | | | GrUrUrArUrCrAmAmCmUmUmGmAmAm |
| | | | AmAmAmGmUmGmGmCmAmCmCmGmA |
| | | | mGmUmCmGmGmUmGmCmU*mU*mU* |
| | | | mU |
| | 3′ | 1449 | mA*mA*mU*rCrUrUrUrGrUrArCrArUrArA |
| | | | rArGrArArGrGrUrUrUrUrArGrAmGmCmU |
| | | | mAmGmAmAmAmUmAmGmCrArArGrUr |
| | | | UrArArArArUrArArGrGrCrUrArGrUrCrCrG |
| | | | rUrUrArUrCrAmAmCmUmUmGmAmAmA |
| | | | mAmAmGmUmGmGmCmAmCmCmGmA |
| | | | mGmUmCmGmGmUmGmCmU*mU*mU* |
| | | | mU |
| 74/300 | 5′ | 1478 | mA*mA*mA*rUrArUrArUrArArArArCrAr |
| | | | ArArGrCrUrCrGrUrUrUrUrArGrAmGmCm |
| | | | UmAmGmAmAmAmUmAmGmCrArArGrU |
| | | | rUrArArArArUrArArGrGrCrUrArGrUrCrCr |
| | | | GrUrUrArUrCrAmAmCmUmUmGmAmAm |
| | | | AmAmAmGmUmGmGmCmAmCmCmGmA |
| | | | mGmUmCmGmGmUmGmCmU*mU*mU* |
| | | | mU |
| | 3′ | 1450 | mU*mA*mC*rArArArArArCrCrUrUrCrUrA |
| | | | rUrArCrArGrGrUrUrUrUrArGrAmGmCmU |
| | | | mAmGmAmAmAmUmAmGmCrArArGrUr |
| | | | UrArArArArUrArArGrGrCrUrArGrUrCrCrG |
| | | | rUrUrArUrCrAmAmCmUmUmGmAmAmA |
| | | | mAmAmGmUmGmGmCmAmCmCmGmA |
| | | | mGmUmCmGmGmUmGmCmU*mU*mU* |
| | | | mU |
| 73/288 | 5′ | 1477 | mA*mU*mA*rUrArArArArCrArArArGrCrU |
| | | | rCrUrGrGrCrGrUrUrUrUrArGrAmGmCmU |
| | | | mAmGmAmAmAmUmAmGmCrArArGrUr |
| | | | UrArArArArUrArArGrGrCrUrArGrUrCrCrG |
| | | | rUrUrArUrCrAmAmCmUmUmGmAmAmA |
| | | | mAmAmGmUmGmGmCmAmCmCmGmA |
| | | | mGmUmCmGmGmUmGmCmU*mU*mU* |
| | | | mU |
| | 3′ | 1449 | mA*mA*mU*rCrUrUrUrGrUrArCrArUrArA |
| | | | rArGrArArGrGrUrUrUrUrArGrAmGmCmU |
| | | | mAmGmAmAmAmUmAmGmCrArArGrUr |
| | | | UrArArArArUrArArGrGrCrUrArGrUrCrCrG |
| | | | rUrUrArUrCrAmAmCmUmUmGmAmAmA |
| | | | mAmAmGmUmGmGmCmAmCmCmGmA |
| | | | mGmUmCmGmGmUmGmCmU*mU*mU* |
| | | | mU |
| 72/288 | 5′ | 1476 | mA*mG*mC*rUrCrUrGrGrCrArGrGrCrArA |
| | | | rUrGrCrUrUrGrUrUrUrUrArGrAmGmCmU |
| | | | mAmGmAmAmAmUmAmGmCrArArGrUr |
| | | | UrArArArArUrArArGrGrCrUrArGrUrCrCrG |
| | | | rUrUrArUrCrAmAmCmUmUmGmAmAmA |
| | | | mAmAmGmUmGmGmCmAmCmCmGmA |
| | | | mGmUmCmGmGmUmGmCmU*mU*mU* |
| | | | mu |
| | 3′ | 1449 | mA*mA*mU*rCrUrUrUrGrUrArCrArUrArA |
| | | | rArGrArArGrGrUrUrUrUrArGrAmGmCmU |
| | | | mAmGmAmAmAmUmAmGmCrArArGrUr |
| | | | UrArArArArUrArArGrGrCrUrArGrUrCrCrG |
| | | | rUrUrArUrCrAmAmCmUmUmGmAmAmA |
| | | | mAmAmGmUmGmGmCmAmCmCmGmA |
| | | | mGmUmCmGmGmUmGmCmU*mU*mU* |
| | | | mU |
| 72/300 | 5′ | 1476 | mA*mG*mC*rUrCrUrGrGrCrArGrGrCrArA |
| | | | rUrGrCrUrUrGrUrUrUrUrArGrAmGmCmU |
| | | | mAmGmAmAmAmUmAmGmCrArArGrUr |
| | | | UrArArArArUrArArGrGrCrUrArGrUrCrCrG |
| | | | rUrUrArUrCrAmAmCmUmUmGmAmAmA |
| | | | mAmAmGmUmGmGmCmAmCmCmGmA |
| | | | mGmUmCmGmGmUmGmCmU*mU*mU* |
| | | | mU |
| | 3′ | 1450 | mU*mA*mC*rArArArArArCrCrUrUrCrUrA |
| | | | rUrArCrArGrGrUrUrUrUrArGrAmGmCmU |
| | | | mAmGmAmAmAmUmAmGmCrArArGrUr |
| | | | UrArArArArUrArArGrGrCrUrArGrUrCrCrG |
| | | | rUrUrArUrCrAmAmCmUmUmGmAmAmA |
| | | | mAmAmGmUmGmGmCmAmCmCmGmA |
| | | | mGmUmCmGmGmUmGmCmU*mU*mU* |
| | | | mU |
| 61/288 | 5′ | 1479 | mA*mC*mG*rUrGrGrCrGrUrGrArArCrArU |
| | | | rCrUrGrCrCrGrUrUrUrUrArGrAmGmCmU |
| | | | mAmGmAmAmAmUmAmGmCrArArGrUr |
| | | | UrArArArArUrArArGrGrCrUrArGrUrCrCrG |
| | | | rUrUrArUrCrAmAmCmUmUmGmAmAmA |
| | | | mAmAmGmUmGmGmCmAmCmCmGmA |
| | | | mGmUmCmGmGmUmGmCmU*mU*mU* |
| | | | mU |
| | 3′ | 1449 | mA*mA*mU*rCrUrUrUrGrUrArCrArUrArA |
| | | | rArGrArArGrGrUrUrUrUrArGrAmGmCmU |
| | | | mAmGmAmAmAmUmAmGmCrArArGrUr |
| | | | UrArArArArUrArArGrGrCrUrArGrUrCrCrG |
| | | | rUrUrArUrCrAmAmCmUmUmGmAmAmA |
| | | | mAmAmGmUmGmGmCmAmCmCmGmA |
| | | | mGmUmCmGmGmUmGmCmU*mU*mU* |
| | | | mU |
| 61/300 | 5′ | 1479 | mA*mC*mG*rUrGrGrCrGrUrGrArArCrArU |
| | | | rCrUrGrCrCrGrUrUrUrUrArGrAmGmCmU |
| | | | mAmGmAmAmAmUmAmGmCrArArGrUr |
| | | | UrArArArArUrArArGrGrCrUrArGrUrCrCrG |
| | | | rUrUrArUrCrAmAmCmUmUmGmAmAmA |
| | | | mAmAmGmUmGmGmCmAmCmCmGmA |
| | | | mGmUmCmGmGmUmGmCmU*mU*mU* |
| | | | mU |
| | 3′ | 1450 | mU*mA*mC*rArArArArArCrCrUrUrCrUrA |
| | | | rUrArCrArGrGrUrUrUrUrArGrAmGmCmU |
| | | | mAmGmAmAmAmUmAmGmCrArArGrUr |
| | | | UrArArArArUrArArGrGrCrUrArGrUrCrCrG |
| | | | rUrUrArUrCrAmAmCmUmUmGmAmAmA |
| | | | mAmAmGmUmGmGmCmAmCmCmGmA |
| | | | mGmUmCmGmGmUmGmCmU*mU*mU* |
| | | | mU |

All bases are in upper case. Lowercase “r” represents RNA, 2′-hydroxy; bases not modified by an “r” are DNA. Lowercase “m” represents RNA with a 2′-methyl group. Underlined = 2′ F modified nucleotide.
All bases are linked via standard phosphodiester bonds except as noted:
“*” represents phosphorothioate modification.

### 8.18 Example 17: Identification of Off-Target Editing Sites for Proximal-ARE gRNA Pair

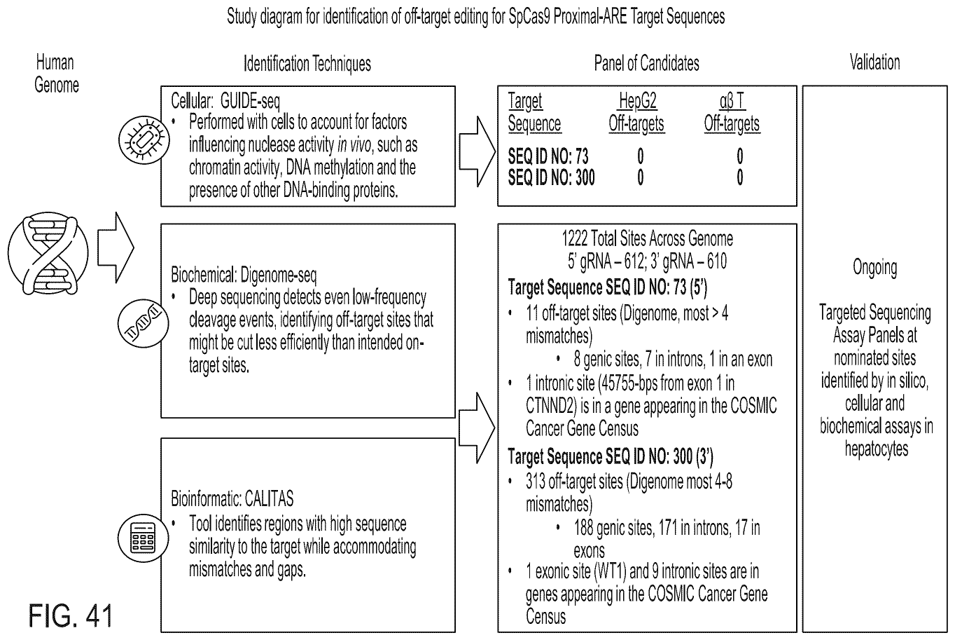

In vitro and in silico methods were used to identify off-target editing sites by the gRNA pair with target sequence SEQ ID NOs: 73 and 300 (FIG. 41). Specifically, GUIDE-seq (cellular), Digenome-seq (biochemical), and CALITAS (bioinformatic)methods were used. No off-target editing sites were identified using GUIDE-seq in either HepG2 cells or αβ T-cells. A total of 1222 potential off-target sites were identified using Digenome and CALITAS. Potential off-target sites are subsequently validated using targeted sequencing assay panels for primary human hepatocytes (PHHs).

### 8.19 Example 18: Determination of SpCas9 Proximal-ARE Dual Guide Impact on Total LDLR Protein Expression, and LDL-C Uptake in Heterozygous Familial Hypercholesterolemia (HeFH) Patient-Derived Fibroblasts

Cells were derived from patients having Heterozygous Familial Hypercholesterolemia. The disease-associated genotype present in each cell population was determined (Table 34).

Cells were treated with the AsCas9 Proximal-ARE gRNA pair at a 1:1:1 ratio of SpCas9 or AsCas12a mRNA: first gRNA: second gRNA LNPs for 4 days. ALC0315 LNPs were formulated encapsulating mRNA encoding SpCas9 (SEQ ID NO: 1480) and gRNAs (Target Sequence SEQ ID NOs: 73 and 300). SpCas9 gRNAs were manufactured with modification patterns as shown in SEQ ID NOs: 1477 and 1450 (Table 33C).

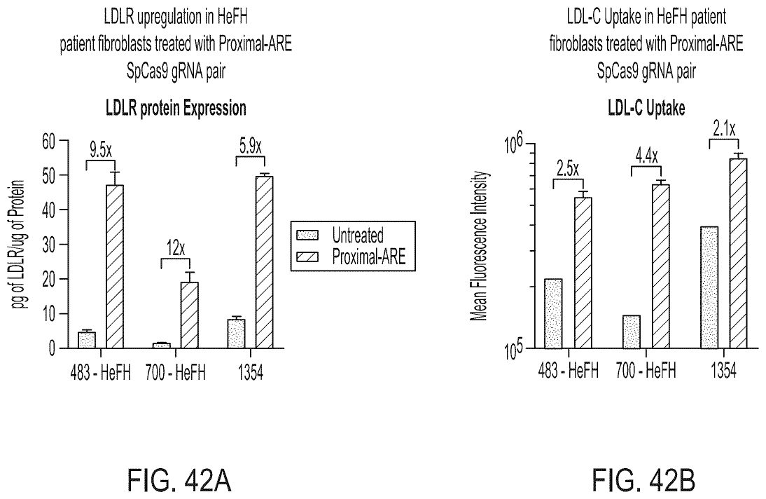

Total LDLR levels were quantified by ELISA. Editing with the SpCas9 gRNA pair inducing the Proximal-ARE mutation increased LDLR protein levels by up to about 12-fold compared with untreated fibroblasts (FIG. 42A). A flow cytometry-based cholesterol uptake assay was used to evaluate LDLR function in edited cells, as described in Example 3. The amount of LDL-C taken up by HeFH patient-derived fibroblasts increased by up to about 4.4-fold compared with untreated fibroblasts (FIG. 42B).

TABLE 34
Disease-associated genotypes of cells
derived from patients having HeFH

| Cell ID | Genotype |
|---|---|
| GM483 | Het-mutation: Cys−>Tyr Exon 14 |
| GM700 | Het-mutation: Cys−>STOP Exon 14 |
| GM1354 | Unknown cause. Possible pathogenic |
| | mutations in Exon 4 and/or Exon 8 |

### 8.20 Example 19: Effects of LDLR 3′ UTR gRNA Pair in Non-Human Primate Studies

A gRNA pair from the in vivo studies described above was further investigated for its effectiveness in non-human primates (NHP). LNPs comprising a GalNAc targeting moiety were formulated encapsulating mRNA encoding SpCas9 (SEQ ID NO: 1480) and two gRNAs (SEQ ID NO: 1477 and 1450) (LNP A). Specifically, purpose-bred Macaca fascicularis (cynomolgus monkeys) were premedicated with dexamethasone, diphenhydramine, and famotidine at 24 hours and at 1 hour prior to LNP dosing. The LNP dose formulations were administered to the animals by intravenous infusion over 2 hours (+10 minutes) on Day 1. LNP dose formulations were administered using a temporary catheter inserted into a peripheral vein connected to an infusion line. The appropriate volume (based on body weight) was delivered using an infusion pump. Subjects were dosed at about 1.5 mg/kg, about 2 mg/kg, about 3 mg/kg, or about 4 mg/kg.

Animals were fasted for at least 4 hours prior to blood collection. Blood was collected into serum separator tubes and processed to serum at two pre-dose time points and at multiple time points out to 29 days.

Serum was analyzed using a clinical chemistry analyzer, and data was collected for multiple parameters, including low density lipoprotein-cholesterol (LDL-C) by direct method and calculated method, total cholesterol, high density lipoprotein (HDL), triglycerides (TG), very low density lipoprotein (VLDL), alanine aminotransferase (ALT), and aspartate aminotransferase (AST). Serum was also analyzed for ApoB by ELISA and Lipoprotein(a) (Lp(a)).

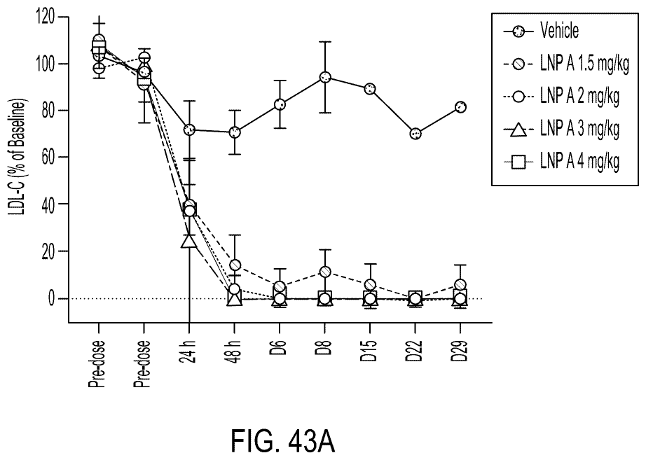

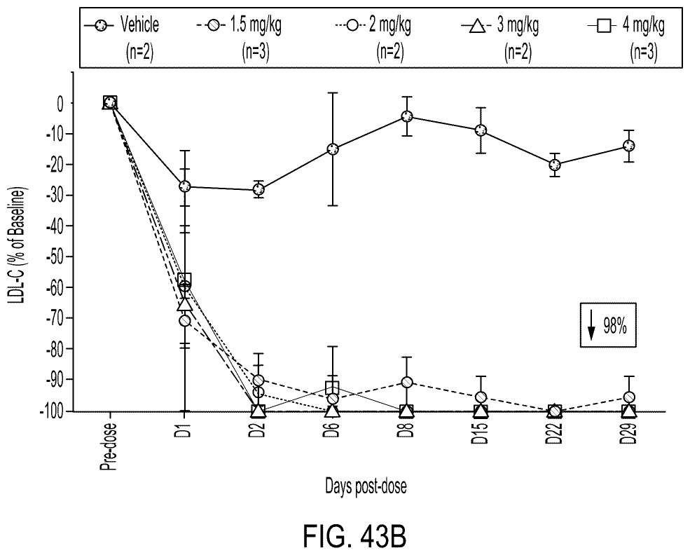

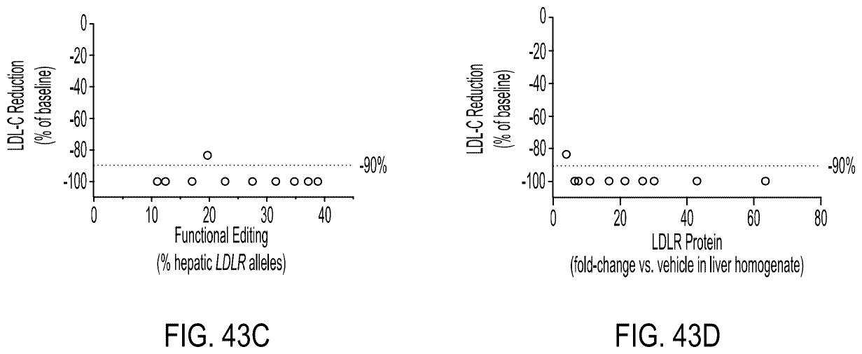

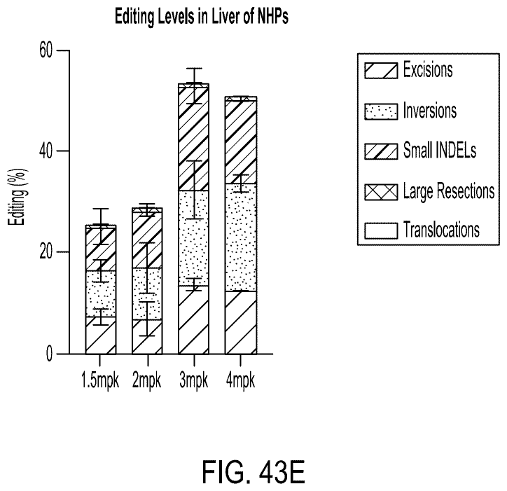

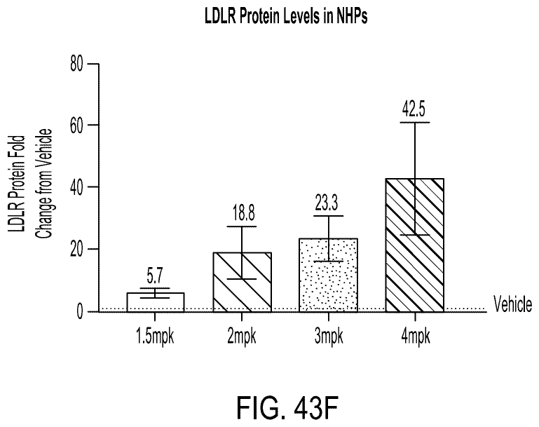

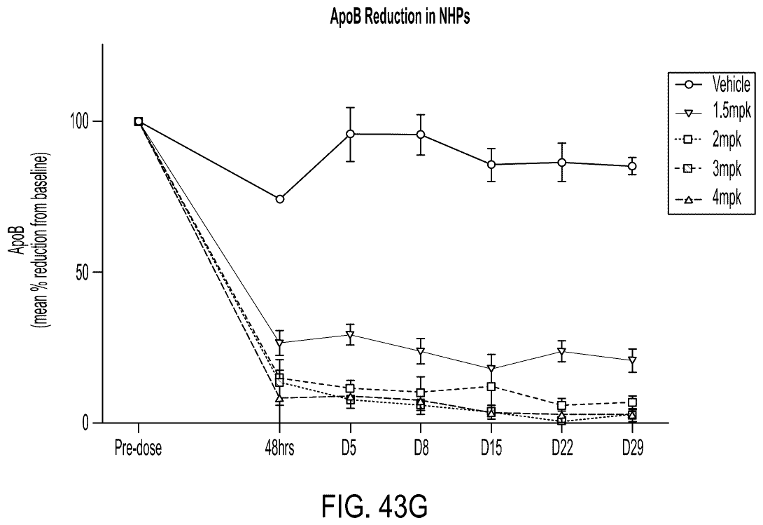

Editing with the SpCas9 Proximal-ARE gRNA pair resulted in a decrease of LDL-C to less than about 20% of baseline LDL-C after about 48 hours post administration for the lowest tested concentration (i.e., 1.5 mg/kg total RNA). Editing with the Proximal-ARE gRNA pair resulted in a decrease of LDL-C to less than about 5% of baseline LDL-C after about 48 hours post administration for all remaining tested concentrations (i.e., 2 mg/kg, 3 mg/kg, and 4 mg/kg total RNA) (FIGS. 43A-43B). Functional editing in about 10% to about 40% of cells resulted in LDL-C reduction of greater than about 90% (FIG. 43C). Liver LDLR protein reduction of greater than about 6-fold resulted in LDL-C reduction of greater than about 90% (FIG. 43D). Editing of the LDLR regulatory region was observed in a dose-dependent fashion, consistent with dual-guide design (FIG. 43E). Treated animals exhibited robust increases in total hepatic LDLR protein (FIG. 43F) accompanied by concordant decreases in ApoB concentrations (FIG. 43G). The reduction in these lipid-associated biomarkers supports a general improvement in lipoprotein metabolism and confirms the functional impact of enhanced LDLR-mediated clearance of atherogenic particles. High density lipoprotein, triglycerides, and very low density lipoprotein remained unchanged. This pharmacodynamic effect was sustained for the duration of observation (4 weeks), consistent with a durable genomic modification and persistent LDLR upregulation.

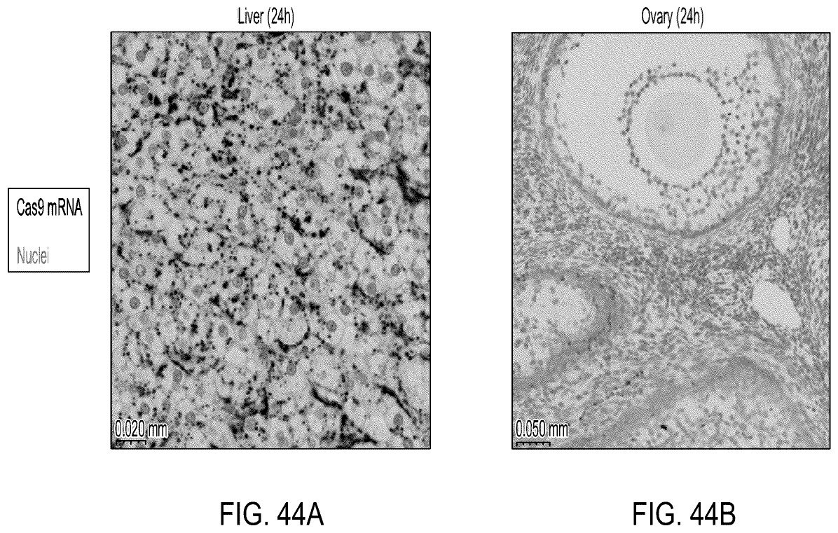

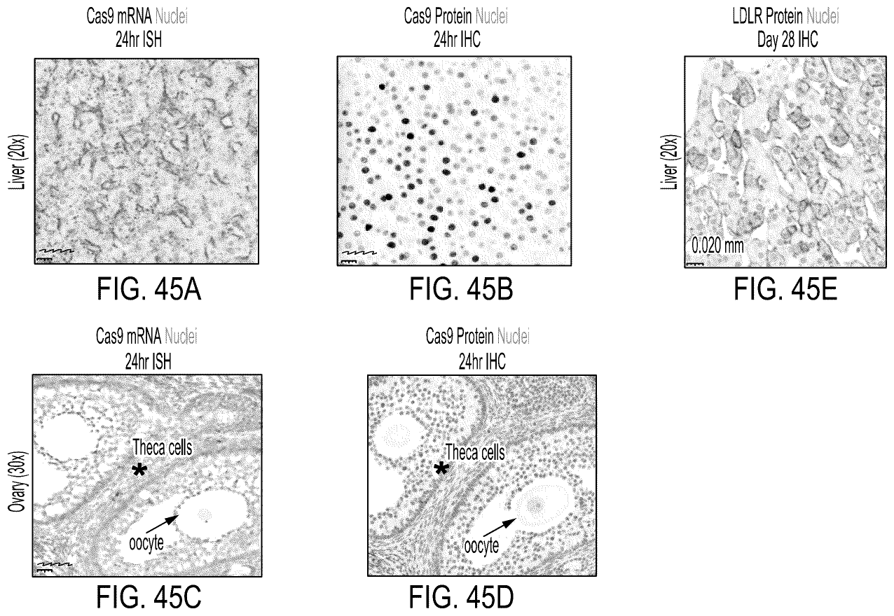

Tissue samples, specifically liver and ovary, were collected from animals treated with 1.5 mg/kg LNP at about 24 hours post-dose. Standard protocols were used to label and detect Cas9 mRNA (by in situ hybridization, i.e., ISH), Cas9 protein (by immunohistochemistry, i.e., IHC) and nuclei (hematoxylin). Widespread delivery of Cas9 mRNA and protein was detected in hepatocytes (FIGS. 44A, 45A, and 45B). Minimal Cas9 mRNA and protein was detected in the ovary; specifically, there was no detectable Cas9 mRNA signal in oocytes and some positivity was detected in theca cells (FIGS. 44B, 45C, and 45D).

Liver samples were collected from animals treated with 1.5 mg/kg LNP at about 28 days post-dose. Standard protocols were used to label and detect LDLR protein (by immunohistochemistry, i.e., IHC) and nuclei (hematoxylin). LDLR protein was abundant in the liver at day 28 post-dose (FIG. 45E).

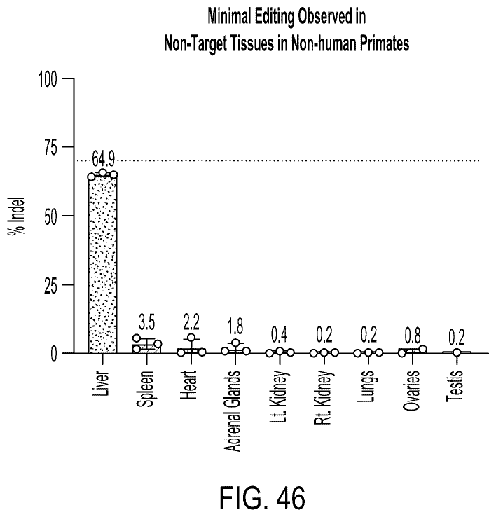

In addition to liver and ovary, several other types of tissue were collected from animals treated with 1.5 mg/kg LNP at about 24 hours post-dose, including spleen, heart, adrenal glands, left kidney, right kidney, lungs, and testis. The frequency of indels induced at the LDLR 3′ UTR was assessed in each tissue using UDiTaS described above/using ILL-Seq. At day 24 post-dose, about 65% of cells in the liver harbored indels, whereas minimal indel generation (about 0-4% of cells) was observed in all other non-target issues (FIG. 46).

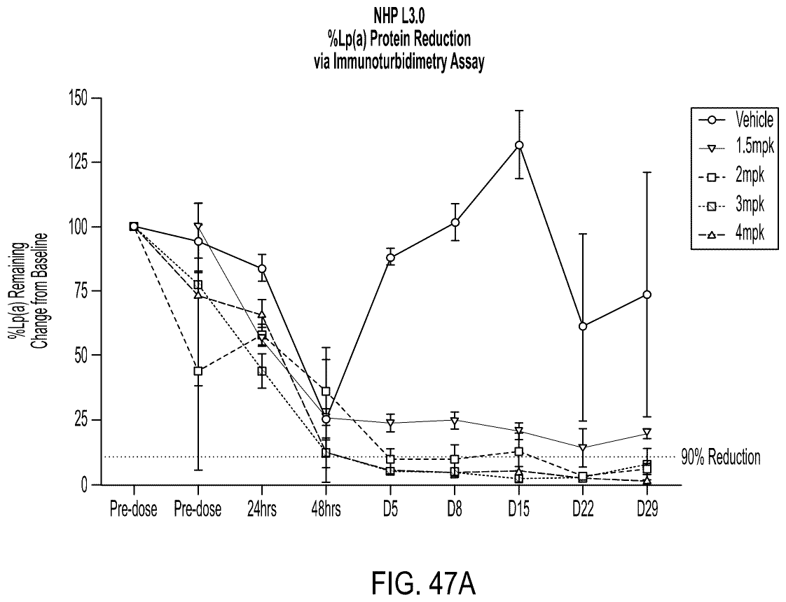

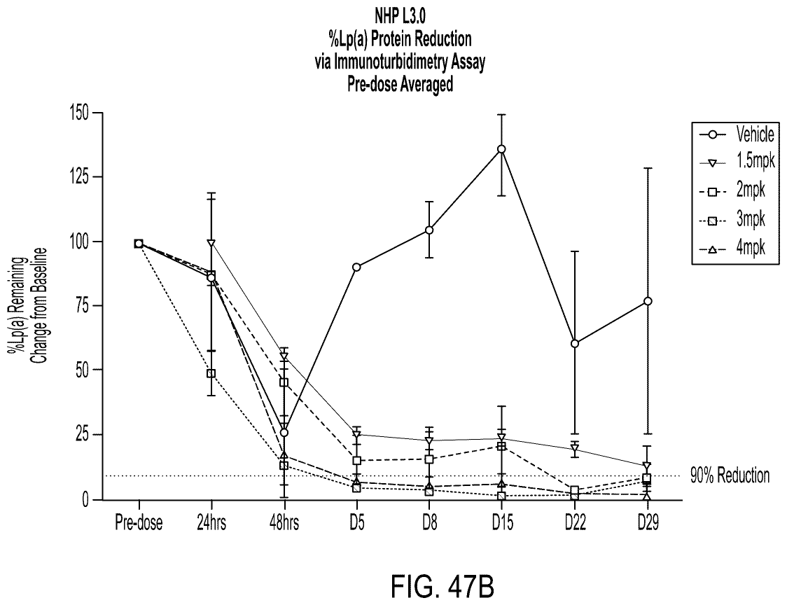

Serum was analyzed for Lp(a) by immunoturbidimetric assay run using a clinical chemistry analyzer, and data was collected for lipoprotein A (Lp(a)) by direct method and calculated method as described in PCT application PCT/2025/166323, filed on Feb. 2, 2025 and incorporated herein by reference. Editing with the SpCas9 Proximal-ARE gRNA pair resulted in a decrease of Lp(a) to less than about 14% of baseline Lp(a) at about 29 days post administration for all tested concentrations (i.e., 1.5 mg/kg, 2 mg/kg, 3 mg/kg, and 4 mg/kg total RNA). The highest tested concentration (i.e., 4 mg/kg total RNA) resulted in about a 95% reduction of Lp(a) compared to baseline Lp(a) at about 29 days post administration (FIGS. 47A-47B).

No adverse clinical observations were noted in treated animals at all dose levels. Transient increases in alanine aminotransferase (ALT), and aspartate aminotransferase (AST) were observed which generally resolved by Day 8. No significant change in high-density lipoprotein (HDL), triglyceride (TG), or very low density lipoprotein (VLDL) were observed.

In summary, treatment resulted in a ≥6-fold increase in LDLR protein in the liver of NHPs. This upregulation strategy led to a concurrent and rapid reduction of ≥90% LDL-C and >90% Lp(a). Administration was well tolerated with no major adverse events and demonstrated an acceptable clinical chemistry, hematology, and coagulation profile. These findings demonstrate preclinical validation for robust lowering of LDL-C and Lp(a) in NHPs. This in vivo strategy represents a potentially transformative, one-time treatment for simultaneously lowering LDL-C and Lp(a) to significantly reduce cardiovascular risk.

1.-7. (canceled)

8. A composition comprising:
a first gRNA molecule comprising a first targeting domain that binds to a first target sequence of an LDLR gene, and
a second gRNA molecule comprising a second targeting domain that binds to a second target sequence of an LDLR gene,
wherein the first and second target sequences of the LDLR gene flank the first AU-rich element (ARE1 element) of the 3′ untranslated region (UTR) of the LDLR gene.

9. The composition of claim 8, wherein the first targeting domain comprises a sequence selected from the group consisting of any one of SEQ ID NOs: 1452, 305-318, 1451, and 1454, and wherein the second targeting domain comprises a sequence selected from the group consisting of any one of SEQ ID NOs: 1475, 519-542, 544-546, 1456, 1470, and 1474.

10. The composition claim 9, wherein the first gRNA molecule comprises a sequence selected from the group consisting of any one of SEQ ID NOS: 1477, 900-913, 1476, 1478, and 1479 and wherein the second gRNA molecule comprises a sequence selected from the group consisting of any one of SEQ ID NOs: 1450, 1114-1137, 1139-1141, 1449, 1460, and 1471.

11. (canceled)

12. (canceled)

13. A genome editing system comprising:
the composition of claim 10, or a nucleic acid molecule encoding the first gRNA molecule and the second gRNA molecule of the composition of claim 10; and
an RNA-guided nuclease, or a nucleic acid encoding the RNA-guided nuclease.

14. The genome editing system of claim 13, wherein the nucleic acid encoding the RNA-guided nuclease is an RNA.

15. The genome editing system of claim 13, wherein the RNA-guided nuclease is selected from the group consisting of Cas9, Cas12a (Cpf1), Cas12b, Cas12c, Cas12h, Cas12i, CasX, CasY, and CasΦ.

16. The genome editing system of claim 13, wherein the RNA-guided nuclease is a Cas9 nuclease.

17. The genome editing system of claim 16, wherein the Cas9 nuclease is a Streptococcus pyogenes Cas9 (SpCas9) nuclease.

18. The genome editing system of claim 13, wherein the RNA-guided nuclease is a Cas12a nuclease.

19. The genome editing system of claim 18, wherein the Cas12a nuclease is a modified Cas12a nuclease.

20. The genome editing system of claim 19, wherein the modified Cas12a nuclease is a modified Acidaminococcus sp. Cas12a (AsCas12a) nuclease.

21. The genome editing system of claim 13, wherein the RNA-guided nuclease comprises an amino acid sequence selected from the group consisting of SEQ ID NOs: 1338-1347.

22. The genome editing system of claim 13, wherein the nucleic acid encoding the RNA-guided nuclease comprises a nucleotide sequence selected from the group consisting of SEQ ID NOs: 1350-1359 and 1480.

23. The genome editing system of claim 13, wherein the first gRNA molecule comprises
a first targeting domain comprising SEQ ID NO: 1452,
wherein the second gRNA molecule comprises a second targeting domain comprising SEQ ID NO: 1475, and
an RNA molecule encoding an RNA-guided nuclease.

24. The genome editing system of claim 23, wherein the first gRNA molecule comprises a sequence of SEQ ID NO: 1477; and wherein the second gRNA molecule comprises a sequence selected from the group consisting of any one of SEQ ID NO: 1450.

25. (canceled)

26. (canceled)

27. (canceled)

28. (canceled)

29. The genome editing system of claim 24, wherein the first targeting domain targets a first target sequence, wherein the second targeting domain targets a second target sequence, and wherein the first target sequence and the second target sequence comprise a first and second nucleotide sequence, respectively, of
SEQ ID NO: 73 and SEQ ID NO: 300.

30. (canceled)

31. (canceled)

32. (canceled)

33. (canceled)

34. (canceled)

35. The genome editing system of claim 24, wherein the genome editing system increases LDLR expression in a target cell by at least about 7-fold as compared to LDLR expression in a control cell or in the target cell prior to contacting with the genome editing system, optionally wherein the LDLR expression is increased in the target cell by at least about 8-fold, at least about 9-fold, or at least about 10-fold, further optionally wherein the LDLR expression is increased by about 7-fold to about 11-fold, about 8-fold to about 10-fold, or about 9-fold to about 10-fold.

36. (canceled)

37. A delivery system for delivering the genome editing system of claim 24, wherein the delivery system comprises the first and second gRNA molecules and the RNA molecule encoding the RNA-guided nuclease.

38. The delivery system of claim 37, wherein the delivery system comprises a lipid nanoparticle (LNP) encapsulating the first and second gRNA molecules and the RNA molecule encoding the RNA-guided nuclease.

39. The delivery system of claim 38, wherein the LNP comprises a targeting moiety.

40. The delivery system of claim 39, wherein the targeting moiety is one or more N-acetylgalactosamine (GalNAc) or GalNAc derivatives.

41. The delivery system of claim 38, wherein the LNP comprises an ionizable lipid, a polyethylene glycol (PEG) lipid, a helper lipid, a sterol, or any combination thereof.

42. A method of editing an LDLR gene in a target cell, the method comprising contacting the target cell with the genome editing system of claim 24.

43.-48. (canceled)

49. A method of treating a disease or a disorder in a subject, the method comprising administering to the subject the genome editing system of claim 24, thereby treating the disease or disorder.

50.-65. (canceled)
